SEQUENCE LISTING (5 sequences)

SEQ ID NO: 1 - protein, 169 aa, Artificial Sequence
  Synthetic polypeptide
  MISC_FEATURE (1)..(169)
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 2 - protein, 188 aa, Artificial Sequence [sequence omitted]

SEQ ID NO: 3 - protein, 188 aa, Artificial Sequence [sequence omitted]

SEQ ID NO: 4 - protein, 188 aa, Artificial Sequence
  Synthetic polypeptide
  MISC_FEATURE (1)..(188)
MAHHHHHHAGGAENLYFQSMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 5 - protein, 170 aa, Artificial Sequence
  Synthetic polypeptide
  MISC_FEATURE (1)..(170)
SMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

(19) United States
(12) Patent Application Publication
(10) Pub. No.: US 2021/0292330 A1
(43) Pub. Date: Sep. 23, 2021

(54) PYRROLIDINE-FUSED HETEROCYCLES

(71) Applicant: Erasca, Inc., San Diego, CA (US)

(72) Inventors: Takasuke Mukaiyama, San Diego, CA (US); Peter C. Chua, San Diego, CA (US); Jean-Michel Vernier, San Diego, CA (US)

(21) Appl. No.: 17/187,541

(22) Filed: Feb. 26, 2021

Related U.S. Application Data
(60) Provisional application No. 62/983,288, filed on Feb. 28, 2020.
(60) Provisional application No. 63/116,146, filed on Nov. 19, 2020.

(51) Int. Cl. C07D 487/04; C07D 519/00

(52) U.S. Cl. CPC C07D 487/04; C07D 519/00

(57) ABSTRACT

A compound of Formula (I) is provided:

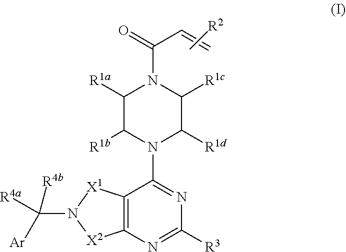

where the variables are defined herein.

### CROSS REFERENCE TO RELATED APPLICATIONS

This application claims priority to U.S. Provisional Application Ser. No. 62/983,288, filed Feb. 28, 2020 and U.S. Provisional Application Ser. No. 63/116,146, filed Nov. 19, 2020, each of which are incorporated herein by reference in their entirety.

### BACKGROUND

Embodiments herein relate to compounds and methods for the treatment of RAS-mediated disease. In particular, embodiments herein relate to compounds and methods for treating diseases such as cancer via targeting oncogenic mutants of the K-RAS isoform.

Ras proteins are small guanine nucleotide-binding proteins that act as molecular switches by cycling between active GTP-bound and inactive GDP-bound conformations. Ras signaling is regulated through a balance between activation by guanine nucleotide exchange factors (GEFs), most commonly son of sevenless (SOS), and inactivation by GTPase-activating proteins (GAPs) such as neurofibromin or p120GAP. The Ras proteins play an important role in the regulation of cell proliferation, differentiation, and survival. Dysregulation of the Ras signaling pathway is almost invariably associated with disease. Hyper-activating somatic mutations in Ras are among the most common lesions found in human cancer. Most of these mutations have been shown to decrease the sensitivity of Ras to GAP stimulation and decrease its intrinsic GTPase activity, leading to an increase in the active GTP-bound population. Although mutation of any one of the three Ras isoforms (K-Ras, N-Ras, or H-Ras) has been shown to lead to oncogenic transformation, K-Ras mutations are by far the most common in human cancer. For example, K-Ras mutations are known to be often associated with pancreatic, colorectal and non-small-cell lung carcinomas. Similarly, H-Ras mutations are common in cancers such as papillary thyroid cancer, lung cancers and skin cancers. Finally, N-Ras mutations occur frequently in hepatocellular carcinoma.

Thus, there is a need in the art for effective Ras inhibitors, which may provide a new class of anticancer compounds. These and other advantages will be apparent to those skilled in the art.

### SUMMARY

In some aspects, embodiments disclosed herein relate to compounds of Formula (I):

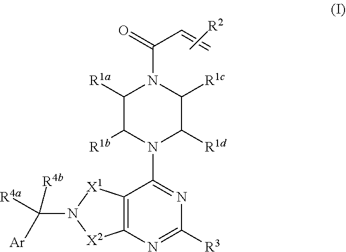

wherein:

X¹ and X² are independently selected from CH₂, carbonyl (—C═O), and CRR′, where R and R′ are independently selected from hydrogen, alkyl, trifluoromethyl, and alkyl optionally substituted with halogen, cyano, hydroxyl, cycloalkyl or heterocycloalkyl; wherein the cycloalkyl or heterocycloalkyl is optionally substituted, fused with an aryl group, or combinations thereof;

Ar is selected from the group consisting of aryl, arylalkyl, arylcarbonyl, arylsulfonyl, heteroaryl, heteroarylalkyl, heteroaryl carbonyl, and heteroarylsulfonyl, any of which may be optionally substituted with halogens, hydroxyl, alkoxyl, alkyl;

R^{1a}, R^{1b}, R^{1c}, and R^{1d} are independently selected from hydrogen, cyano, alkyl, hydroxy alkyl, alkynylalkyl and cyanoalkyl;

R² is selected from the group consisting of hydrogen, fluorine, methyl, and —CH₂NR^{a}R^{b}, wherein R^{a} and R^{b} are independently selected from hydrogen or alkyl; or R^{a} and R^{b} combine to form a C₂-C₆ nitrogen containing heterocycle;

R³ is selected from hydrogen, alkyl, alkoxy, amino, aminoalkylamino, halogen, heterocyclylalkoxy, aminoalkoxy, N-alkylaminoalkoxy, N,N-dialkylaminoalkoxy, mercapto-alkyl, mercapto aryl, aryl, any of which may be optionally substituted; and R^{4a} and R^{4b} are independently selected from hydrogen, aryl, alkyl, trifluoroalkyl, alkyl optionally with halogen and cycloalkyl; or one of R^{4a} and R^{4b} forms a fused, non-aromatic ring structure with Ar; or R^{4a} and R^{4b} together define a double-bonded oxygen (carbonyl).

In some aspects, embodiments herein relate to methods of treating a subject with cancer associated with a G12C Kras mutation comprising administering to the subject a compound, as disclosed herein, in a pharmaceutically acceptable vehicle.

### DETAILED DESCRIPTION

### I. General

Disclosed herein are potent and selective tricyclic quinazoline-2-ones compounds, which have been found to be useful as inhibitors of oncogenic mutants of RAS proteins. Among various advantages, the compounds disclosed herein are selective for oncogenic RAS mutants over wild-type RAS proteins. Further, compounds disclosed herein may exhibit selectivity for oncogenic mutants of K-RAS over other mutated K-RAS proteins, as well as mutants of the N-RAS and H-RAS isoforms. In particular, the compounds disclosed herein may exhibit selectivity for K-RAS, N-RAS, and H-RAS mutants having a common G12C mutation. Also disclosed herein are pharmaceutical compositions comprising these compounds, and their application in the treatment of disease, such as cancer. Methods of inhibition of oncogenic mutant K-RAS, N-RAS, and H-RAS activity are also provided, as well as methods for the treatment of oncogenic mutant RAS-mediated diseases, especially those involving elevated levels of oncogenic mutated RAS, in particular cancer.

Disclosed herein is a class of compounds useful in treating oncogenic mutant K-RAS-mediated disorders and conditions, defined by structural Formula (I):

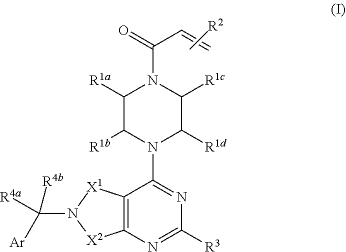

wherein:

X¹ and X² are independently selected from CH₂, carbonyl (—C═O), and CRR′, where R and R′ are independently selected from hydrogen, alkyl, trifluoromethyl, and alkyl optionally substituted with halogen, cyano, hydroxyl, cycloalkyl or heterocycloalkyl; wherein the cycloalkyl or heterocycloalkyl is optionally substituted, fused with an aryl group, or combinations thereof;

Ar is selected from the group consisting of aryl, arylalkyl, arylcarbonyl, arylsulfonyl, heteroaryl, heteroarylalkyl, heteroaryl carbonyl, and heteroarylsulfonyl, any of which may be optionally substituted with halogens, hydroxyl, alkoxyl, alkyl;

R^{1a}, R^{1b}, R^{1c}, and R^{1d} are independently selected from hydrogen, cyano, alkyl, hydroxy alkyl, alkynylalkyl and cyanoalkyl;

R² is selected from the group consisting of hydrogen, fluorine, methyl, and —CH₂NR^{a}R^{b}, wherein R^{a} and R^{b} are independently selected from hydrogen or alkyl; or R^{a} and R^{b} combine to form a C₂-C₆ nitrogen containing heterocycle;

R³ is selected from hydrogen, alkyl, alkoxy, amino, aminoalkylamino, halogen, heterocyclylalkoxy, aminoalkoxy, N-alkylaminoalkoxy, N,N-dialkylaminoalkoxy, mercapto-alkyl, mercapto aryl, aryl, any of which may be optionally substituted; and R^{4a} and R^{4b} are independently selected from hydrogen, aryl, alkyl, trifluoroalkyl, alkyl optionally with halogen and cycloalkyl; or one of R^{4a} and R^{4b} forms a fused, non-aromatic ring structure with Ar; or R^{4a} and R^{4b} together define a double-bonded oxygen (carbonyl).

Compounds according to the various embodiments disclosed herein possess useful oncogenic mutant RAS inhibiting or modulating activity, and may be used in the treatment or prophylaxis of a disease or condition in which oncogenic mutant RAS plays an active role. Thus, in a broad aspect, embodiments disclosed herein also provide pharmaceutical compositions comprising one or more compounds disclosed herein together with a pharmaceutically acceptable carrier, as well as methods of making and using the compounds and compositions. Embodiments disclosed herein provide methods for selectively inhibiting the RAS that are oncogenic mutants having the G12C mutation. In some embodiments, there are provided methods for treating an oncogenic mutant K-RAS-mediated disorder in a subject, comprising administering to the subject a therapeutically effective amount of a compound or pharmaceutical composition according to the various embodiments disclosed herein. Related embodiments disclose the use of the compounds disclosed herein as therapeutic agents, for example, in treating cancer and other diseases involving elevated levels of oncogenic mutant K-RAS. The various embodiments disclosed herein also contemplate the use of the compounds disclosed herein for use in the manufacture of a medicament for the treatment of a disease or condition ameliorated by the inhibition of oncogenic mutant K-RAS. In some such embodiments, the disease or condition is cancer. Each of the aforementioned methods apply equally to the similar mutation in N-RAS and H-RAS bearing the G12C mutation.

Compounds of the various embodiments disclosed herein may be selective amongst the RAS oncogenic mutant forms in various ways. For example, compounds described herein may be selective for G12C mutants of K-RAS, N-RAS, or H-RAS. In certain embodiments, compounds of the various embodiments disclosed herein may be selective for K-RAS G12C over other K-RAS mutants and Wild Type K-RAS. Likewise, compounds of various embodiments disclosed herein may be selective for N-RAS and H-RAS bearing the same G12C mutation.

The various embodiments disclosed herein also relate to methods of inhibiting at least one RAS function comprising the step of contacting an oncogenic mutant RAS with a compound of Formula I, as described herein. The cell phenotype, cell proliferation, activity of the mutant RAS, change in biochemical output produced by active mutant RAS, expression of mutant RAS, or binding of mutant RAS with a natural binding partner may be affected. Such methods may be embrace modes of treatment of disease, biological assays, cellular assays, biochemical assays, or the like.

### II. Definitions

### A. General Definitions

As used herein, the terms below have the meanings indicated.

When ranges of number values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure, taking into account significant figures.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

### B. Chemical Definitions

The following chemical functional group definitions are provided to give guidance in understanding their meaning and scope. Those skilled in the art will recognize that these functional groups are being used in a manner consistent with practice of the chemical arts. Any of the following chemical functional groups may be optionally substituted as defined below and each chemical functional group below may itself be an optional substitution.

The term “acyl,” as used herein, alone or in combination, refers to a carbonyl (C═O) attached to an alkenyl, alkyl, aryl, cycloalkyl, heteroaryl, heterocycloalkyl, or any other moiety were the atom attached to the carbonyl is carbon. An “acetyl” group, which is a type of acyl, refers to a (—C(═O)CH₃) group. An “alkylcarbonyl” or “alkanoyl” group refers to an alkyl group attached to the parent molecular moiety through a carbonyl group. Examples of such groups include, without limitation, methylcarbonyl and ethylcarbonyl. Similarly, an “arylcarbonyl” or “aroyl” group refers to an aryl group attached to the parent molecular moiety through a carbonyl group. Examples of such groups include, without limitation, benzoyl and naphthoyl. Accordingly, generic examples of acyl groups include alkanoyl, aroyl, heteroaroyl, and so on. Specific examples of acyl groups include, without limitation, formyl, acetyl, acryloyl, benzoyl, trifluoroacetyl and the like.

The term “alkenyl,” as used herein, alone or in combination, refers to a straight-chain or branched-chain hydrocarbon radical having one or more double bonds and containing from 2 to 20 carbon atoms. In certain embodiments, the alkenyl may comprise from 2 to 6 carbon atoms, or from 2 to 4 carbons, either of which may be referred to as “lower alkenyl.” The term “alkenylene” refers to a carbon-carbon double bond system attached at two or more positions such as ethenylene (—CH═CH—). Alkenyl can include any number of carbons, such as C₂, C₂₋₃, C₂₋₄, C₂₋₅, C₂₋₆, C₂₋₇, C₂₋₈, C₂₋₉, C2U0, C₃, C₃₋₄, C₃₋₅, C₃₋₆, C₄, C₄₋₅, C₄₋₆, C₅, C₅₋₆, and C₆, and so on up to 20 carbon atoms. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted. Unless otherwise specified, the term “alkenyl” may include “alkenylene” groups.

The term “alkoxy,” as used herein, alone or in combination, refers to an alkyl ether radical, wherein the term alkyl is as defined below. Alkoxy groups may have the general formula: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, and the like. The alkoxy groups can be further optionally substituted as defined herein.

The term “alkyl,” as used herein, alone or in combination, (sometimes abbreviated Alk) refers to a straight-chain or branched-chain alkyl radical containing from 1 to 20 carbon atoms. In certain embodiments, the alkyl may comprise from 1 to 10 carbon atoms. In further embodiments, the alkyl may comprise from 1 to 6 carbon atoms, or from 1 to 4 carbon atoms. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. When an alkyl is a methyl group, it can be displayed in the context of a structure as the abbreviation Me, —CH₃, or simply as a line. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino (—NHMe), N-ethylamino (—NHEt), N,N-dimethylamino (—NMe₂), N,N-ethylmethylamino (—NMeEt) and the like. The term “aminoalkyl” refers to reverse orientation in which the amino group appears distal to the parent molecular moiety and attachment to the parent molecular moiety is through the alkyl group. For example, NH₂(CH₂)ₙ—describes an aminoalkyl group with a terminal amine at the end of an alkyl group attached to the parent molecular moiety. The two terms alkylamino and aminoalkyl can be combined to describe an “alkylaminoalkyl” group in which an alkyl group resides on a nitrogen atom distal to the parent molecular moiety, such as MeNH(CH₂)ₙ—. In a similar manner, an aryl group, as defined herein, may combine in a similar fashion providing an arylaminoalkyl group ArNH(CH₂)ₙ—. For additional clarity nomenclature may be provided where the group that is attached to nitrogen is indicated so by use of “N-” in the name, such as N-arylaminoalkyl, which is understood to mean that the aryl group is a substituent on the nitrogen atom of the aminoalkyl group, the alkyl being attached the parent molecular moiety.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (AlkS—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like. Similarly, “arylthio” refers to arylthioether (ArS—) radical wherein the term aryl is as defined herein and wherein the sulfur may be singly or double oxidized.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted. Unless otherwise specified, the term “alkynyl” may include “alkynylene” groups.

The terms “amido,” as used herein, alone or in combination, refer to an amino group as described below attached to the parent molecular moiety through a carbonyl group. The term “C-amido” as used herein, alone or in combination, refers to a —C(═O)N(R)₂ group where is R as defined herein. The term “N-amido” as used herein, alone or in combination, refers to RC(═O)N(R′)— group, with R and R′ as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —N(R)(R′) or —N⁺(R)(R′)(R″), wherein R, R′ and R″ are independently selected from the group consisting of hydrogen, alkyl, acyl, heteroalkyl, aryl, cycloalkyl, heteroaryl, and heterocycloalkyl, any of which may themselves be optionally substituted.

The term “amino acid,” as used herein, alone or in combination, means a substituent of the form —NRCH(R′)C(O)OH, wherein R is typically hydrogen, but may be cyclized with N (for example, as in the case of the amino acid proline), and R′ is selected from the group consisting of hydrogen, alkyl, heteroalkyl, cycloalkyl, heterocycloalkyl, aryl, heteroaryl, amino, amido, cycloalkylalkyl, heterocycloalkylalkyl, arylalkyl, heteroarylalkyl, aminoalkyl, amidoalkyl, hydroxyalkyl, thiol, thioalkyl, alkylthioalkyl, and alkylthio, any of which may be optionally substituted. The term “amino acid” includes all naturally occurring amino acids as well as synthetic analogues.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, naphthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄— derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid (oxygen) end, and which may be optionally substituted as defined herein.

The term “O-carbamyl” as used herein, alone or in combination, refers to a —OC(O)NRR′, group, with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(═O)H] and in combination is a —C(═O)— group.

The term “carboxyl” or “carboxyl,” as used herein, refers to —C(═O)OH, O-carboxy, C-carboxy, or the corresponding “carboxylate” anion, such as is in a carboxylic acid salt. An “O-carboxy” group refers to a RC(═O)O— group, where R is as defined herein. A “C-carboxy” group refers to a —C(═O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl radical wherein each cyclic moiety contains from 3 to 12 carbon atom ring members and which may optionally be a benzo fused ring system which is optionally substituted as defined herein. In some embodiments, a cycloalkyl may comprise from 3 to 7 carbon atoms, or from 5 to 7 carbon atoms. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1.1.1]pentane, camphor, adamantane, and bicyclo[3.2.1]octane.

The term “electrophilic moiety,” as used herein, is used in accordance with its plain ordinary chemical meaning and refers to a chemical group that is electrophilic. Exemplary electrophilic moieties include, without limitation, unsaturated carbonyl containing compounds such as acrylamides, acrylates, unsaturated (i.e., vinyl) sulfones or phosphates, epoxides, and vinyl epoxides.

The term “ester,” as used herein, alone or in combination, refers to a carboxyl group bridging two moieties linked at carbon atoms (—CRR′C(═O)OCRR′—), where each R and R′ are independent and defined herein.

The term “ether,” as used herein, alone or in combination, typically refers to an oxy group bridging two moieties linked at carbon atoms. “Ether” may also include polyethers, such as, for example, ˜RO(CH₂)₂O(CH₂)₂O(CH₂)₂OR′, ˜RO(CH₂)₂O(CH₂)₂OR′, —RO(CH₂)₂OR′, and —RO(CH₂)₂OH.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl, trihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethyl ene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized (i.e. bond to 4 groups). The heteroatom(s) O, N and S may be placed at any interior position of the heteroalkyl group. Up to two heteroatoms may be consecutive, such as, for example, —CH₂NHOCH₃. The term heteroalkyl may include ethers.

The term “heteroaryl,” as used herein, alone or in combination, refers to 3 to 7 membered unsaturated heteromonocyclic rings, or fused polycyclic rings, each of which is 3 to 7 membered, in which at least one of the fused rings is unsaturated, wherein at least one atom is selected from the group consisting of O, S, and N. In some embodiments, a heteroaryl may comprise from 5 to 7 carbon atoms. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Non-limiting examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

Heteroaryl groups can include any number of ring atoms, such as, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

The terms “heterocycloalkyl” and, interchangeably, “heterocycle,” or “heterocyclyl” as used herein, alone or in combination, each refer to a saturated, partially unsaturated, or fully unsaturated monocyclic, bicyclic, or tricyclic heterocyclic radical containing at least one heteroatom as ring members, wherein each heteroatom may be independently selected from the group consisting of nitrogen, oxygen, and sulfur. In certain embodiments, a heterocycloalkyl may comprise from 1 to 4 heteroatoms as ring members. In further embodiments, a heterocycloalkyl may comprise from 1 to 2 heteroatoms ring members. In some embodiments, a heterocycloalkyl may comprise from 3 to 8 ring members in each ring. In further embodiments, a heterocycloalkyl may comprise from 3 to 7 ring members in each ring. In yet further embodiments, a heterocycloalkyl may comprise from 5 to 6 ring members in each ring. “Heterocycloalkyl” and “heterocycle” are intended to include sugars, sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycloalkyl groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, epoxy, isoindolinyl, morpholinyl, piperazinyl, pyrrolidinyl, tetrahydropyridinyl, piperidinyl, thiomorpholinyl, and the like. The heterocycloalkyl groups may be optionally substituted unless specifically prohibited.

“Heterocycloalkyl” may refer to a saturated ring system having from 3 to 12 ring members and from 1 to 5 heteroatoms of N, O and S. The heteroatoms can also be oxidized, such as, but not limited to, S(O) and S(O)₂. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, 4, or 5, or 1 to 2, 1 to 3, 1 to 4, 1 to 5, 2 to 3, 2 to 4, 2 to 5, 3 to 4 or 3 to 5. The heterocycloalkyl group can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. The heterocycloalkyl group can include groups such as aziridine, azetidine, pyrrolidine, piperidine, azepane, diazepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline, diazabicycloheptane, diazabicyclooctane, diazaspirooctane or diazaspirononane. Heterocycloalkyl groups can be unsubstituted or substituted. For example, heterocycloalkyl groups can be substituted with C₁₋₆ alkyl or oxo (═O), among many others. Heterocycloalkyl groups can also include a double bond or a triple bond, such as, but not limited to dihydropyridine or 1,2,3,6-tetrahydropyridine.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxazolidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

The term “hydrazinyl” as used herein, alone or in combination, refers to two amino groups joined by a single bond, i.e., —N—N—. In general, the hydrazinyl group has optional substitution on at least one NH hydrogen to confer stability.

The term “hydroxamic acid” or its ester as used herein, refers to —C(O)ON(R)O(R′), wherein R and R′ are as defined herein, or the corresponding “hydroxamate” anion, including any corresponding hydroxamic acid salt.

The term “hydroxy,” as used herein, alone or in combination, refers to OH.

The term “hydroxyalkyl,” as used herein, alone or in combination, refers to a hydroxy group attached to the parent molecular moiety through an alkyl group. “Hydroxyalkyl” or “alkylhydroxy” refers to an alkyl group, as defined above, where at least one of the hydrogen atoms is replaced with a hydroxy group. As for the alkyl group, hydroxyalkyl or alkylhydroxy groups can have any suitable number of carbon atoms, such as C₁₋₆. Exemplary C₁₋₄ hydroxyalkyl groups include, but are not limited to, hydroxymethyl, hydroxyethyl (where the hydroxy is in the 1 or 2 position), hydroxypropyl (where the hydroxy is in the 1, 2 or 3 position), hydroxybutyl (where the hydroxy is in the 1, 2, 3 or 4 position), 1,2dihydroxyethyl, and the like.

The term “imino,” as used herein, alone or in combination, refers to C═NR.

The term “iminohydroxy,” as used herein, alone or in combination, refers to C═N(OH) and it O-ether C═N—OR.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “linking group,” as used herein refers to any nitrogen containing organic fragment that serves to connect the pyrimidine or pyridone core of the compounds disclosed herein to the electrophilic moiety E, as defined herein. Exemplary linking groups include piperazines, aminoalkyls, alkyl- or aryl-based diamines, aminocycloalkyls, amine-containing spirocyclics, any of which may be optionally substituted as defined herein. In some embodiments, linking groups may comprise the substructure L-Q-L′-E wherein Q is a monocyclic 4 to 7 membered ring or a bicyclic, bridged, or fused, or spiro 6-11 membered ring, any of which optionally include one or more nitrogen atoms, E is the electrophilic group, L is bond, C₁₋₆ alkylene, —O—C₀₋₅ alkylene, —S—C₀₋₅ alkylene, or —NH—C₀₋₅ alkylene, and for C₂₋₆ alkylene, —O—C₂₋₅ alkylene, —S—C₂₋₅ alkylene, and NH—C₂₋₅ alkylene, one carbon atom of any of the alkylene groups can optionally be replaced with O, S, or NH; and L′ is bond when Q comprises a nitrogen to link to E, otherwise L′ is NR, where R is hydrogen or alkyl.

The term “lower,” as used herein, alone or in combination, means containing from 1 to and including 6 carbon atoms, or from 1 to 4 carbon atoms.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “phosphoamide” as used herein, alone or in combination, refers to a phosphate group [(OH)₂P(═O)O—] in which one or more of the hydroxyl groups has been replaced by nitrogen, amino, or amido.

The term “phosphonate” as used herein, alone or in combination, refers to a group of the form ROP(OR′)(OR)O— wherein R and R′ are selected from the group consisting of hydrogen, alkyl, acyl, heteroalkyl, aryl, cycloalkyl, heteroaryl, and heterocycloalkyl, any of which may themselves be optionally substituted. “Phosphonate” includes “phosphate [(OH)₂P(O)O—] and related phosphoric acid anions which may form salts.

The terms “sulfonate,” “sulfonic acid,” and “sulfonic,” as used herein, alone or in combination, refers to the —SO₃H group and its anion as the sulfonic acid is used in salt formation or sulfonate ester where OH is replaced by OR, where R is not hydrogen, but otherwise is as defined herein, and typically being alkyl or aryl.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(═S)H and in combination is a —C(═S)— group.

The term “N-thiocarbamyl” refers to an ROC(═S)NR′— group, with R and R′ as defined herein.

The term “O-thiocarbamyl” refers to a —OC(═S)NRR′, group with R and R′ as defined herein.

The term “thiocyanato” refers to a —CNS group.

The term “trihalomethanesulfonamido” refers to a X₃CS(═O)₂NR— group with X is a halogen and R as defined herein.

The term “trihalomethanesulfonyl” refers to a X₃CS(═O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethylsilyl, tert-butyldimethylsilyl, triphenyl silyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent. A “null” group occurring between two other group may also be understood to be a collapsing of flanking groups. For example, if in —(CH₂)ₓG¹G²G³, the element G² were null, said group would become —(CH₂)ₓG¹G³.

The term “optionally substituted” means the anteceding group or groups may be substituted or unsubstituted. Groups constituting optional substitution may themselves be optionally substituted. For example, where an alkyl group is embraced by an optional substitution, that alkyl group itself may also be optionally substituted. When substituted, the substituents of an “optionally substituted” group may include, without limitation, one or more substituents independently selected from the following groups or a particular designated set of groups, alone or in combination: alkyl, alkenyl, alkynyl, alkanoyl, heteroalkyl, heterocycloalkyl, haloalkyl, haloalkenyl, haloalkynyl, lower perhaloalkyl, perhaloalkoxy, cycloalkyl, phenyl, aryl, aryloxy, alkoxy, haloalkoxy, oxo, acyloxy, carbonyl, carboxyl, alkylcarbonyl, carboxyester, carboxamido, cyano, hydrogen, halogen, hydroxy, amino, alkylamino, arylamino, amido, nitro, thiol, alkylthio, haloalkylthio, perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, carbamate, and urea. Particular subsets of optional substitution include, without limitation: (1) alkyl, halo, and alkoxy; (2) alkyl and halo; (3) alkyl and alkoxy; (4) alkyl, aryl, and heteroaryl; (5) halo and alkoxy; and (6) hydroxyl, alkyl, halo, alkoxy, and cyano. Where an optional substitution comprises a heteroatom-hydrogen bond (—NH—, SH, OH), further optional substitution of the heteroatom hydrogen is contemplated and includes, without limitation optional substitution with alkyl, acyl, alkoxymethyl, alkoxyethyl, arylsulfonyl, alkyl sulfonyl, any of which are further optionally substituted. These subsets of optional substitutions are intended to be merely exemplary and any combination of 2 to 5, or 2 to 10, or 2 to 20 of the groups recited above up to all the group recited above and any subrange in between are contemplated. “Optionally substituted” may include any of the chemical functional groups defined hereinabove and throughout this disclosure. Two optional substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃).

The various optional substitutions need not be the same and any combination of optional substituent groups may be combined. For example, a carbon chain may be substituted with an alkyl group, a halo group, and an alkoxy group. Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, hydroxyl, halogen, alkyl, cycloalkyl, heteroalkyl, aryl, heteroaryl and heterocycloalkyl, any of which may be optionally substituted. Each such R and R′ groups should be understood to be optionally substituted as defined herein. Each incidence of R and R′ should be understood to be independent. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers, axial asymmetry (non-interchanging rotamers), or the like may exist in the compounds of the various embodiments disclosed herein. Such chirality may be designated by the symbols “R” or “S,” depending on the configuration of substituents around the chiral carbon atom or the relevant axis. It should be understood that embodiments encompasses all stereochemical isomeric forms, including diastereomeric, enantiomeric, and epimeric forms, d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the various embodiments disclosed herein may exist as geometric isomers. The various embodiments disclosed herein includes all cis, trans, syn, anti, entgegen (E), and zusammen (Z) isomers as well as the appropriate mixtures thereof. Additionally, compounds may exist as tautomers, including keto-enol tautomers; all tautomeric isomers are embraced by the embodiments disclosed herein.

Additionally, the compounds of the various embodiments disclosed herein can exist in unsolvated as well as solvated forms with pharmaceutically acceptable solvents such as water, ethanol, and the like. In general, the solvated forms are considered equivalent to the unsolvated forms for the purposes of the various embodiments disclosed herein.

The term “bond” refers to a covalent linkage between two atoms, or two moieties when the atoms joined by the bond are considered part of larger substructure. A bond may be single, double, or triple unless otherwise specified. A dashed line between two atoms in a drawing of a molecule indicates that an additional bond may be present or absent at that position.

1. Salts of Compounds

The compounds disclosed herein can exist as therapeutically acceptable salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the various embodiments disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the various embodiments disclosed herein contemplates sodium, potassium, magnesium, and calcium salts of the compounds disclosed herein, and the like.

Basic addition salts can be prepared during the final isolation and purification of the compounds by reacting a carboxyl group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

### C. Treatment-Related Definitions

The term “disease” as used herein is intended to be generally synonymous, and is used interchangeably with, the terms “disorder” and “condition” (as in medical condition), in that all reflect an abnormal condition of the body or of one of its parts that impairs normal functioning and is typically manifested by distinguishing signs and symptoms.

As used herein, the term “cancer” refers to all types of cancer, neoplasm or malignant tumors found in mammals (e.g., but not limited to, humans), including leukemia, lymphomas, carcinomas and sarcomas. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Stemberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi'ssarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifomi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, Schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

“Ras associated cancer” (also referred to herein as “Ras related cancer”) refers to a cancer caused by aberrant Ras activity or signaling. A “cancer associated with aberrant K-Ras activity” (also referred to herein as “K-Ras related cancer”) is a cancer caused by aberrant K-Ras activity or signaling (e.g. a mutant K-Ras). K-Ras related cancers may include lung cancer, non-small cell lung cancer, breast cancer, leukemia, pancreatic cancer, colon cancer, colorectal cancer. Other cancers that are associated with aberrant activity of one or more of Ras, K-Ras, H-Ras, N-Ras, mutant K-Ras (including K-Ras G12C, K-Ras G12V, K-Ras G13C, K-Ras G12D, K-Ras G13D mutants), mutant N-Ras, and mutant H-Ras are well known in the art and determining such cancers are within the skill of a person of skill in the art.

The term “administer (or administering) a Ras inhibitor” means administering a compound that inhibits the activity or level (e.g. amount) or level of a signaling pathway of one or more Ras proteins (e.g. a Ras inhibitor, K-Ras inhibitor, N-Ras inhibitor, H-Ras inhibitor, mutant K-Ras inhibitor, K-Ras G12C inhibitor, K-Ras G12V inhibitor, K-Ras G13C inhibitor, K-Ras G12D inhibitor, K-Ras G13D inhibitor) to a subject. Administration may include, without being limited by mechanism, allowing sufficient time for the Ras inhibitor to reduce the activity of one or more Ras proteins or for the Ras inhibitor to reduce one or more symptoms of a disease (e.g. cancer, wherein the Ras inhibitor may arrest the cell cycle, slow the cell cycle, reduce DMA replication, reduce cell replication, reduce cell growth, reduce metastasis, or cause ceil death). The term “administer (or administering) a K-Ras inhibitor” means administering a compound that inhibits the activity or level (e.g. amount) or level of a signaling pathway of one or more K-Ras proteins (K-Ras, mutant K-Ras, K-Ras G12C, K-Ras G12V, K-Ras G12D, K-Ras G13C, K-Ras G13D). In embodiments, the administering does not include administration of any active agent other than the recited active agent.

The term“associated” or“associated with” in the context of a substance or substance activity or function associated with a disease (e.g. Ras (e.g., human K-Ras or human H-Ras) activity, a protein associated disease, a cancer associated with aberrant Ras activity, K-Ras associated cancer, mutant K-Ras associated cancer, activated K-Ras associated cancer, K-RasG12C associated cancer, K-Ras G12V associated cancer, K-Ras G13C associated cancer, K-Ras G12D associated cancer, K-Ras G13D associated cancer) means that the disease (e.g. cancer) is caused by (in whole or in part), or a symptom of the disease is caused by (in whole or inpart) the substance or substance activity or function. For example, a cancer associated with aberrant Ras activity or function may be a cancer that results (entirely or partially) from aberrant Ras activity or function (e.g. enzyme activity, protein-protein binding, signaling pathway) or a cancer wherein a particular symptom of the disease is caused (entirely or partially) by aberrant Ras activity or function. As used herein, what is described as being associated with a disease, if a causative agent, could be a target for treatment of the disease. For example, a cancer associated with aberrant Ras activity or function or a Ras associated cancer, may be treated with a Ras modulator or Ras inhibitor, in the instance where increased Ras activity or function (e.g., signaling pathway activity) causes the cancer. For example, a cancer associated with K-Ras G12C may be a cancer that a subject with K-Ras G12C is at higher risk of developing as compared to a subject without K-Ras G12C. For example, a cancer associated with K-Ras G12V may be a cancer that a subject with K-Ras G12V is at higher risk of developing as compared to a subject without K-Ras G12V.

The term “Ras” refers to one or more of the family of human Ras GTPase proteins (e.g. K-Ras, H-Ras, N-Ras). The term “K-Ras” refers to the nucleotide sequences or proteins of human K-Ras (e.g. human K-Ras4A (NP_203524.1), human K-Ras4B (NP_004976.2), or both K-Ras4A and K-Ras4B). The term “K-Ras” includes both the wild-type form of the nucleotide sequences or proteins as well as any mutants thereof. In some embodiments, “K-Ras” is wild-type K-Ras. In some embodiments, “K-Ras” is one or more mutant forms. The term “K-Ras” XYZ refers to a nucleotide sequence or protein of a mutant K-Ras wherein the Y numbered amino acid of K-Ras that has an X amino acid in the wildtype instead has a Z amino acid in the mutant (e.g. K-Ras G12C has a G in wildtype protein but a C in the K-Ras G12C mutant protein). In some embodiments K-Ras refers to K-Ras4A and K-Ras4B. In some embodiments, K-Ras refers to K-Ras4A. In some embodiments, K-Ras refers to K-Ras4B (e.g., NM_004985.4 or NP_004976.2). In some embodiments, K-Ras refers to the protein including (e.g., consisting of) the amino acid sequence below or including the sequence below with one or more mutations (e.g., G12C, G12V, or G13C):

(SEQ ID NO: 1)
MTEYKLVVVGAGGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIP FIETSAKTR
QGVDDAFYTLVREIRKHKEK

In some embodiments, K-Ras refers to the protein including (e.g., consisting of) the amino acid sequence below or including (e.g., consisting of) the sequence below with one or more mutations (e.g., G12C, G12V, or G13C):

(SEQ ID NO: 2)
MTEYKLVVVGAGGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM
(SEQ ID NO: 3)

| | |
|---|---|
| 1 | mteyklvvvg aggvgksalt iqliqnhfvd eydptiedsy |
| | rkqvvidget clldildtag |
| 61 | qeeysamrdq ymrtgegflc vfainntksf edihhyreqi |
| | krvkdsedvp mvlvgnkcdl |
| 121 | psrtvdtkqa qdlarsygip fietsaktrq gvddafytlv |
| | reirkhkekm skdgkkkkkk |
| 181 | sktkcvim |

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“K-RAS inhibitor” is used herein to refer to a compound that exhibits an IC₅₀ with respect to K-RAS activity of no more than about 100 μM and more typically not more than about 50 μM, as measured in the K-RAS assay described generally hereinbelow. “IC₅₀” is that concentration of inhibitor that reduces the activity of an enzyme (e.g., K-RAS) to half-maximal level. Compounds of the various embodiments disclosed herein have been discovered to exhibit inhibition against oncogenic mutant K-RAS isoforms. In some embodiments, compounds will exhibit an IC₅₀ with respect to oncogenic mutant K-RAS of no more than about 10 μM; in further embodiments, compounds will exhibit an IC₅₀ with respect to K-RAS of no more than about 5 μM; in yet further embodiments, compounds will exhibit an IC₅₀ with respect to K-RAS of not more than about 1 μM, as measured in the K-RAS assay described herein. In yet further embodiments, compounds will exhibit an IC₅₀ with respect to K-RAS of not more than about 200 nM.

The phrase “therapeutically effective” is intended to qualify the amount of active ingredients used in the treatment of a disease or disorder. This amount will achieve the goal of reducing or eliminating the disease or disorder.

As used herein, reference to “treatment” of a subject is intended to include prophylaxis. The term “subject” means all mammals, including humans. Examples of subjects include humans, cows, dogs, cats, goats, sheep, pigs, and rabbits. In some embodiments, the subject is a human.

The term “prodrug” refers to a compound that is made active in vivo through chemical reaction in vivo thereby releasing an active compound. Compounds disclosed herein can be modified to exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the active compound. Additionally, prodrugs can be converted to the active compounds by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the active compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug is a compound which is administered as an ester (the “prodrug”), which is then metabolically hydrolyzed to the carboxylic acid, as the active entity. Additional examples include peptidyl derivatives of a compound. The term “therapeutically acceptable prodrug,” refers to those prodrugs or zwitterions which are suitable for use in contact with the tissues of subjects without undue toxicity, irritation, and allergic response, are commensurate with a reasonable benefit/risk ratio, and are effective for their intended use.

### III. Compound Embodiments

A compound of Formula (I):

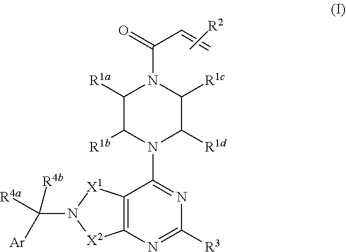

wherein:

X¹ and X² are independently selected from CH₂, carbonyl (—C═O), and CRR′, where R and R′ are independently selected from hydrogen, alkyl, trifluoromethyl, and alkyl optionally substituted with halogen, cyano, hydroxyl, cycloalkyl or heterocycloalkyl; wherein the cycloalkyl or heterocycloalkyl is optionally substituted, fused with an aryl group, or combinations thereof; wherein the cycloalkyl or heterocycloalkyl is optionally substituted, fused with an aryl group, or combinations thereof;

Ar is selected from the group consisting of aryl, arylalkyl, arylcarbonyl, arylsulfonyl, heteroaryl, heteroarylalkyl, heteroaryl carbonyl, and heteroarylsulfonyl, any of which may be optionally substituted with halogens, hydroxyl, alkoxyl, alkyl;

R^{1a}, R^{1b}, R^{1c}, and R^{1d} are independently selected from hydrogen, cyano, alkyl, hydroxy alkyl, alkynylalkyl and cyanoalkyl;

R² is selected from the group consisting of hydrogen, fluorine, methyl, and —CH₂NR^{a}R^{b}, wherein R^{a} and R^{b} are independently selected from hydrogen or alkyl; or R^{a} and R^{b} combine to form a C₂-C₆ nitrogen containing heterocycle;

R³ is selected from hydrogen, alkyl, alkoxy, amino, aminoalkylamino, halogen, heterocyclylalkoxy, aminoalkoxy, N-alkylaminoalkoxy, N,N-dialkylaminoalkoxy, mercapto-alkyl, mercapto aryl, aryl, any of which may be optionally substituted; and

R^{4a} and R^{4b} are independently selected from hydrogen, aryl, alkyl, trifluoroalkyl, alkyl optionally with halogen and cycloalkyl; or one of R^{4a} and R^{4b} forms a fused, non-aromatic ring structure with Ar;

or R^{4a} and R^{4b} together define a double-bonded oxygen (carbonyl).

In embodiments, X¹ is CH₂.

In embodiments, X² CH₂.

In embodiments, R³ is O-linked N-methyl-L-prolinol.

In embodiments, R³ is hydrogen.

In embodiments, R² is hydrogen.

In embodiments, R² is fluorine.

In embodiments, R² is N,N-dimethylaminomethyl.

In embodiments, R^{1b} is methyl.

In embodiments, a stereogenic center created by the R^{1b} methyl group is in the R-configuration.

In embodiments, a stereogenic center created by the R^{1b} methyl group is in the S-configuration.

In embodiments, R^{1c} is methyl.

In embodiments, a stereogenic center created by the R^{1c} methyl group is in the R-configuration.

In embodiments, a stereogenic center created by the R^{1c} methyl group is in the S-configuration.

In embodiments, R^{1a} is cyanomethyl.

In embodiments, a stereogenic center created by the cyanomethyl group is in the R-configuration.

In embodiments, a stereogenic center created by the cyanomethyl group is in the S-configuration.

In embodiments, R^{1d} is hydrogen.

In embodiments, Ar is:

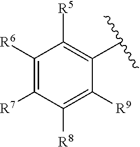

wherein R⁵, R⁶, R⁷, R⁸, and R⁹ are each independently selected from the group consisting of hydrogen, halo, alkyl, alkoxy, haloalkyl, trifluoromethyl, cycloalkyl and any two adjacent R⁵, R⁶, R⁷, R⁸, and R⁹ together combine to form a further fused ring that is an aromatic ring optionally comprising 1 to 3 heteroatoms independently selected from N, O or S, the further fused ring being optionally substituted.

In embodiments, R⁵ and R⁶ combine to form a fused pyrazole, wherein a nitrogen atom of the fused pyrazole is optionally methylated.

In embodiments, Ar is selected from the group consisting of:

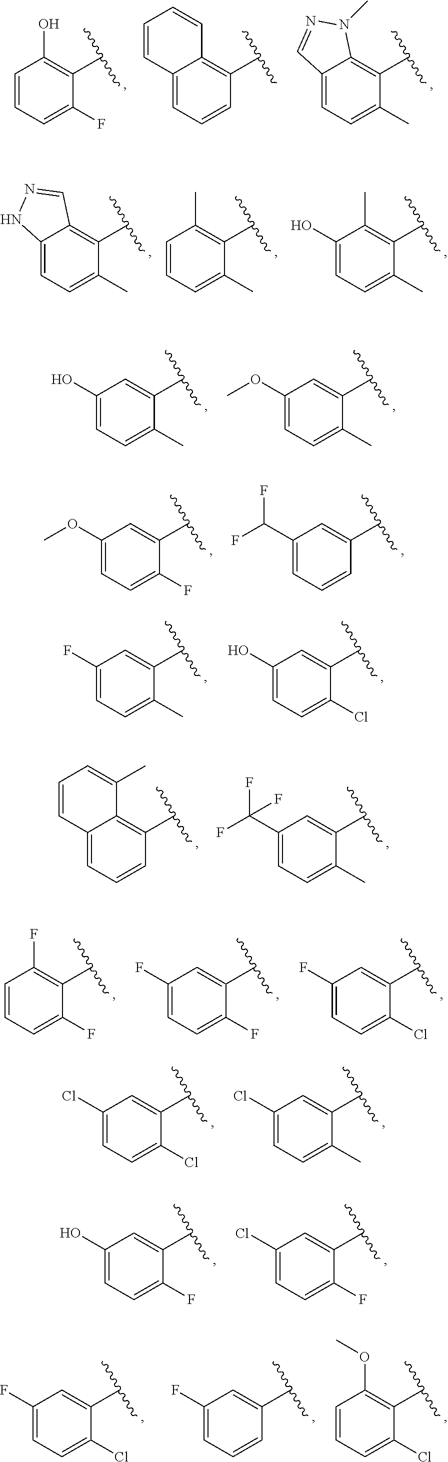

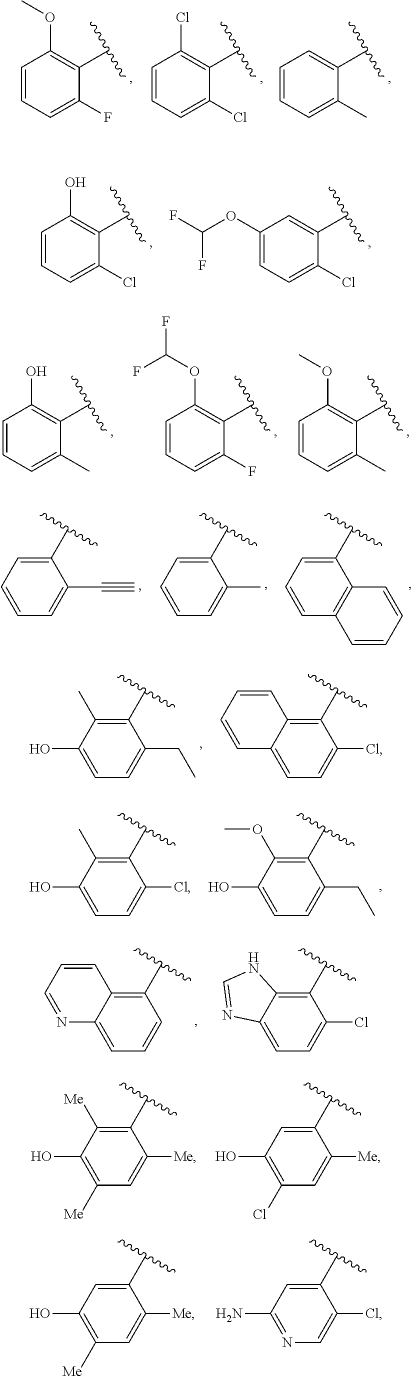

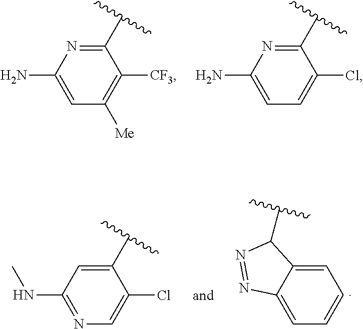

In embodiments, R^{4a} and R^{4b} are hydrogen.

In embodiments, there are provided compounds of Formula (II):

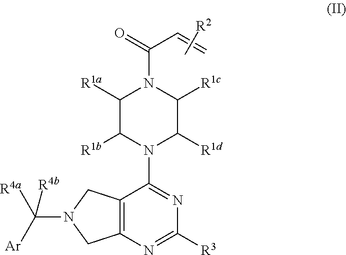

wherein:

Ar is selected from the group consisting of aryl, arylalkyl, arylcarbonyl, arylsulfonyl, heteroaryl, heteroarylalkyl, heteroaryl carbonyl, and heteroarylsulfonyl, any of which may be optionally substituted;

R^{1a}, R^{1b}, R^{1c}, and R^{1d} are independently selected from hydrogen, alkyl, and cyanoalkyl;

R² is selected from the group consisting of hydrogen, fluorine, methyl, and —CH₂NR^{a}R^{b}, wherein R^{a} and R^{b} are independently selected from hydrogen or alkyl; or R^{a} and R^{b} combine to form a C₂-C₆ nitrogen containing heterocycle;

R³ is selected from hydrogen, alkyl, alkoxy, amino, aminoalkylamino, halogen, heterocyclylalkoxy, aminoalkoxy, N-alkylaminoalkoxy, N,N-dialkylaminoalkoxy, any of which may be optionally substituted; and

R^{4a} and R^{4b} are independently selected from hydrogen, aryl, alkyl, trifluoroalkyl, and cycloalkyl; or one of R^{4a} and R^{4b} forms a fused, non-aromatic ring structure with Ar.

In embodiments, R³ is O-linked N-methyl-L-prolinol.

In embodiments, R³ is hydrogen.

In embodiments, R² is hydrogen.

In embodiments, R² is fluorine.

In embodiments, R² is N,N-dimethylaminomethyl.

In embodiments, R^{1b} is methyl.

In embodiments, a stereogenic center created by the R^{1b} methyl group is in the R-configuration.

In embodiments, a stereogenic center created by the R^{1b} methyl group is in the S-configuration.

In embodiments, R^{1c} is methyl.

In embodiments, a stereogenic center created by the R^{1c} methyl group is in the R-configuration.

In embodiments, a stereogenic center created by the R^{1c} methyl group is in the S-configuration.

In embodiments, R^{1a} is cyanomethyl.

In embodiments, a stereogenic center created by the cyanomethyl group is in the R-configuration.

In embodiments, a stereogenic center created by the cyanomethyl group is in the S-configuration.

In embodiments, R^{1d} is hydrogen.

In embodiments, Ar is:

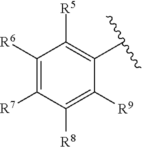

wherein R⁵, R⁶, R⁷, R⁸, and R⁹ are each independently selected from the group consisting of hydrogen, halo, alkyl, alkoxy, haloalkyl, trifluoromethyl, cycloalkyl and any two adjacent R⁵, R⁶, R⁷, R⁸, and R⁹ together combine to form a further fused ring that is an aromatic ring optionally comprising 1 to 3 heteroatoms independently selected from N, O or S, the further fused ring being optionally substituted.

In embodiments, R⁵ and R⁶ combine to form a fused pyrazole, wherein a nitrogen atom of the fused pyrazole is optionally methylated.

In embodiments, Ar is selected from the group consisting of:

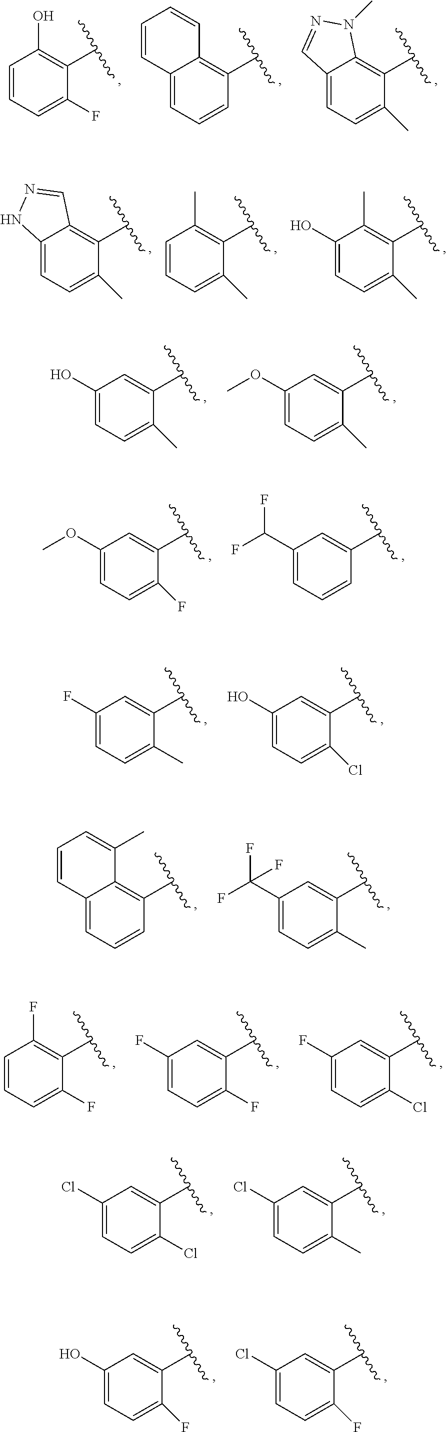

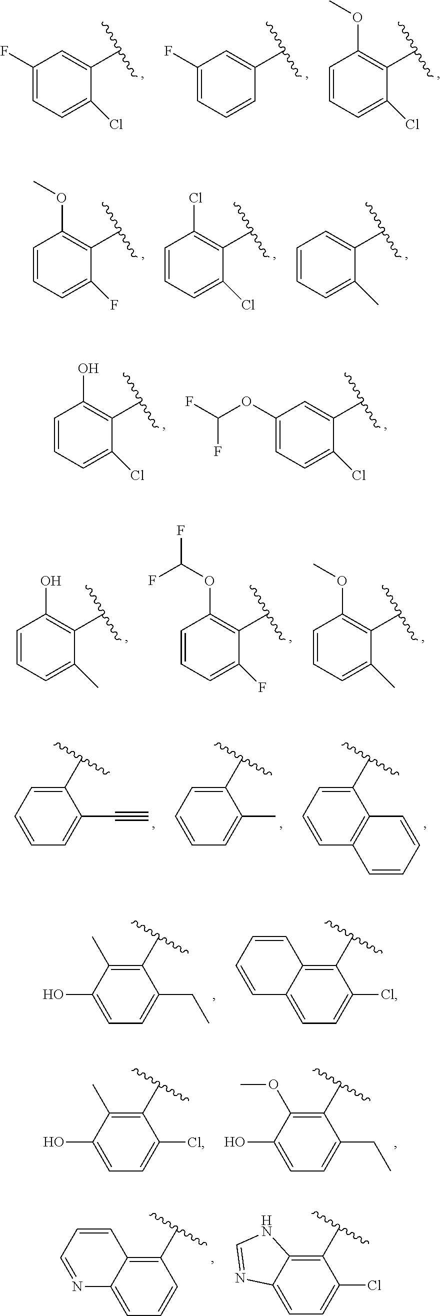

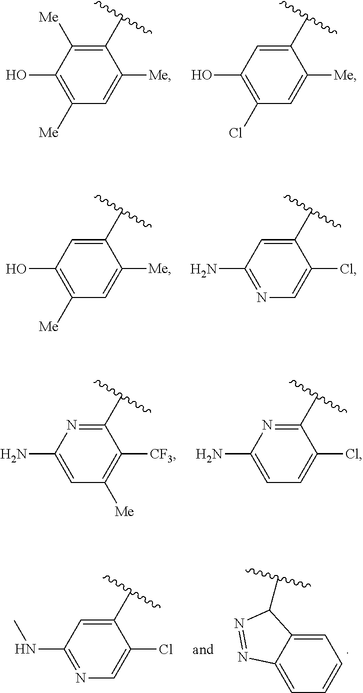

In embodiments, R4a and R4b are hydrogen.

In embodiments, there are provided compounds of Formula (III):

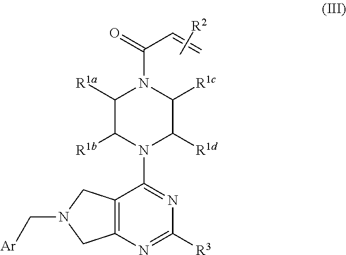

wherein:

Ar is selected from the group consisting of aryl, arylalkyl, arylcarbonyl, arylsulfonyl, heteroaryl, heteroarylalkyl, heteroaryl carbonyl, and heteroarylsulfonyl, any of which may be optionally substituted;

R^{1a}, R^{1b}, R^{1c}, and R^{1d} are independently selected from hydrogen, alkyl, and cyanoalkyl;

R² is selected from the group consisting of hydrogen, fluorine, methyl, and —CH₂NR^{a}R^{b}, wherein R^{a} and R^{b} are independently selected from hydrogen or alkyl; or R^{a} and R^{b} combine to form a C₂-C₆ nitrogen containing heterocycle; and

R³ is selected from hydrogen, alkyl, aminoalkyl, heterocyclylalkyl, N-alkylaminoalkyl, and N,N-dialkylaminoalkyl, any of which may be optionally substituted.

In embodiments, R³ is heterocyclylalkyl.

In embodiments, R³ is N,N-dialkylaminoalkyl.

In embodiments, R² is hydrogen.

In embodiments, R² is fluorine.

In embodiments, R² is N,N-dimethylaminomethyl.

In embodiments, R^{1b} is methyl.

In embodiments, a stereogenic center created by the R^{1b} methyl group is in the R-configuration.

In embodiments, a stereogenic center created by the R^{1b} methyl group is in the S-configuration.

In embodiments, R^{1c} is methyl.

In embodiments, a stereogenic center created by the R^{1c} methyl group is in the R-configuration.

In embodiments, a stereogenic center created by the R^{1c} methyl group is in the S-configuration.

In embodiments, R^{1a} is cyanomethyl.

In embodiments, a stereogenic center created by the cyanomethyl group is in the R-configuration.

In embodiments, a stereogenic center created by the cyanomethyl group is in the S-configuration.

In embodiments, R^{1d} is hydrogen.

In embodiments, Ar is:

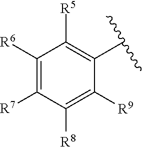

wherein R⁵, R⁶, R⁷, R⁸, and R⁹ are each independently selected from the group consisting of hydrogen, halo, alkyl, alkoxy, haloalkyl, trifluoromethyl, cycloalkyl and any two adjacent R⁵, R⁶, R⁷, R⁸, and R⁹ together combine to form a further fused ring that is an aromatic ring optionally comprising 1 to 3 heteroatoms independently selected from N, O or S, the further fused ring being optionally substituted.

In embodiments, R⁵ and R⁶ combine to form a fused pyrazole, wherein a nitrogen atom of the fused pyrazole is optionally methylated.

In embodiments, there are provided compounds of Formula (IV) or pharmaceutically acceptable salt thereof:

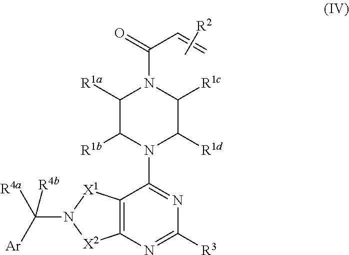

wherein:

X¹ and X² are independently selected from CH₂, carbonyl (—C═O), and CRR′, where R and R′ are independently selected from hydrogen, alkyl, trifluoromethyl, and alkyl optionally substituted with halogen, cyano, hydroxyl, cycloalkyl or heterocycloalkyl; wherein the cycloalkyl or heterocycloalkyl is optionally substituted, fused with an aryl group, or combinations thereof; wherein the cycloalkyl or heterocycloalkyl is optionally substituted, fused with an aryl group, or combinations thereof;

Y is C or S;

Ar is selected from the group consisting of aryl, arylalkyl, arylcarbonyl, arylsulfonyl, heteroaryl, heteroarylalkyl, heteroaryl carbonyl, and heteroarylsulfonyl, any of which may be optionally substituted with halogens, hydroxyl, alkoxyl, alkyl, amidoalkyl;

R^{1a}, R^{1b}, R^{1c}, and R^{1d} are independently selected from hydrogen, cyano, alkyl, amido, alkylamido, CH₂SOₚMe, where p is 0 to 2, heteroarylalkyl, hydroxy alkyl, alkynylalkyl and cyanoalkyl; wherein any C—H present in R^{1a}, R^{1b}, R^{1c}, and R^{1d} is optionally exchanged for C—F; or any two R^{1a}, R^{1b}, R^{1c}, and R^{1d} combine to form to form a fused 3-6-membered ring or a 1 to 4 atom bridging unit, wherein each atom of the fused 3-6-membered ring or the 1 to 4 atom bridging unit comprises, independently, an optionally substituted methylene unit or a heteroatom selected from NR^{N}, O, or S, or SO₂, wherein R^{N} is hydrogen, alkyl, and fluorinated alkyl; and wherein the fused 3-6-membered ring or the 1 to 4 atom bridging unit is optionally substituted with oxo, halogen, and hydroxyl;

R² is selected from the group consisting of hydrogen, fluorine, methyl, and —CH₂NR^{a}R^{b}, wherein R^{a} and R^{b} are independently selected from hydrogen or alkyl; or R^{a} and R^{b} combine to form a C₂-C₆ nitrogen containing heterocycle;

R³ is selected from hydrogen, alkyl, alkoxy, amino, aminoalkylamino, halogen, heterocyclylalkoxy, aminoalkoxy, N-alkylaminoalkoxy, N,N-dialkylaminoalkoxy, mercapto-alkyl, mercapto aryl, aryl, any of which may be optionally substituted; and

R^{4a} and R^{4b} are independently selected from hydrogen, aryl, alkyl, trifluoroalkyl, alkyl optionally with halogen and cycloalkyl; or one of R^{4a} and R^{4b} forms a fused, non-aromatic ring structure with Ar; or when Y is S, R^{4a} and R^{4b} are double bond to O.

or R^{4a} and R^{4b} together define a double-bonded oxygen (carbonyl).

In embodiments, X¹ is CH₂.

In embodiments, X² CH₂.

In embodiments, R³ is O-linked N-methyl-L-prolinol.

In embodiments, R³ is hydrogen.

In embodiments, R² is hydrogen.

In embodiments, R² is fluorine.

In embodiments, R² is N,N-dimethylaminomethyl.

In embodiments, R^{1b} is methyl.

In embodiments, a stereogenic center created by the R^{1b} methyl group is in the R-configuration.

In embodiments, a stereogenic center created by the R^{1b} methyl group is in the S-configuration.

In embodiments, R^{1c} is methyl.

In embodiments, a stereogenic center created by the R^{1c} methyl group is in the R-configuration.

In embodiments, a stereogenic center created by the R^{1c} methyl group is in the S-configuration.

In embodiments, R^{1a} is cyanomethyl.

In embodiments, a stereogenic center created by the cyanomethyl group is in the R-configuration.

In embodiments, a stereogenic center created by the cyanomethyl group is in the S-configuration.

In embodiments, R^{1d} is hydrogen.

In embodiments, Ar is:

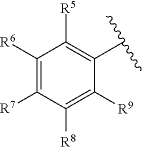

wherein R⁵, R⁶, R⁷, R⁸, and R⁹ are each independently selected from the group consisting of hydrogen, halo, alkyl, alkoxy, haloalkyl, trifluoromethyl, cycloalkyl and any two adjacent R⁵, R⁶, R⁷, R⁸, and R⁹ together combine to form a further fused ring that is an aromatic ring optionally comprising 1 to 3 heteroatoms independently selected from N, O or S, the further fused ring being optionally substituted.

In embodiments, there are provided compounds of Formula (V) or pharmaceutically acceptable salt thereof:

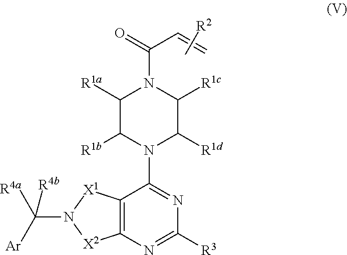

wherein:

X¹ and X² are independently selected from CH₂, carbonyl (—C═O), and CRR′, where R and R′ are independently selected from hydrogen, alkyl, trifluoromethyl, and alkyl optionally substituted with halogen, cyano, hydroxyl, cycloalkyl or heterocycloalkyl; wherein the cycloalkyl or heterocycloalkyl is optionally substituted, fused with an aryl group, or combinations thereof; wherein the cycloalkyl or heterocycloalkyl is optionally substituted, fused with an aryl group, or combinations thereof;

Ar is selected from the group consisting of aryl, arylalkyl, arylcarbonyl, arylsulfonyl, heteroaryl, heteroarylalkyl, heteroaryl carbonyl, and heteroarylsulfonyl, any of which may be optionally substituted with halogens, hydroxyl, alkoxyl, alkyl, amidoalkyl;

R^{1a}, R^{1b}, R^{1c}, and R^{1d} are independently selected from hydrogen, cyano, alkyl, amido, alkylamido, CH₂SOₚMe, where p is 0 to 2, heteroarylalkyl, hydroxy alkyl, alkynylalkyl and cyanoalkyl; wherein any C—H present in R^{1a}, R^{1b}, R^{1c}, and R^{1d} is optionally exchanged for C—F; or any two R^{1a}, R^{1b}, R^{1c}, and R^{1d} combine to form to form a fused 3-6-membered ring or a 1 to 4 atom bridging unit, wherein each atom of the fused 3-6-membered ring or the 1 to 4 atom bridging unit comprises, independently, an optionally substituted methylene unit or a heteroatom selected from NR^{N}, O, or S, or SO₂, wherein R^{N} is hydrogen, alkyl, and fluorinated alkyl; and wherein the fused 3-6-membered ring or the 1 to 4 atom bridging unit is optionally substituted with oxo, halogen, and hydroxy;

R² is selected from the group consisting of hydrogen, fluorine, methyl, and —CH₂NR^{a}R^{b}, wherein R^{a} and R^{b} are independently selected from hydrogen or alkyl; or R^{a} and R^{b} combine to form a C₂-C₆ nitrogen containing heterocycle;

R³ is selected from hydrogen, alkyl, alkoxy, amino, aminoalkylamino, halogen, heterocyclylalkoxy, aminoalkoxy, N-alkylaminoalkoxy, N,N-dialkylaminoalkoxy, mercapto-alkyl, mercapto aryl, aryl, any of which may be optionally substituted; and

R^{4a} and R^{4b} are independently selected from hydrogen, aryl, alkyl, trifluoroalkyl, alkyl optionally with halogen and cycloalkyl; or one of R^{4a} and R^{4b} forms a fused, non-aromatic ring structure with Ar.

or R^{4a} and R^{4b} together define a double-bonded oxygen (carbonyl).

In embodiments, X¹ is CH₂.

In embodiments, X² CH₂.

In embodiments, R³ is O-linked N-methyl-L-prolinol.

In embodiments, R³ is hydrogen.

In embodiments, R² is hydrogen.

In embodiments, R² is fluorine.

In embodiments, R² is N,N-dimethylaminomethyl.

In embodiments, R^{1b} is methyl.

In embodiments, a stereogenic center created by the R^{1b} methyl group is in the R-configuration.

In embodiments, a stereogenic center created by the R^{1b} methyl group is in the S-configuration.

In embodiments, R^{1c} is methyl.

In embodiments, a stereogenic center created by the R^{1c} methyl group is in the R-configuration.

In embodiments, a stereogenic center created by the R^{1c} methyl group is in the S-configuration.

In embodiments, R^{1a} is cyanomethyl.

In embodiments, a stereogenic center created by the cyanomethyl group is in the R-configuration.

In embodiments, a stereogenic center created by the cyanomethyl group is in the S-configuration.

In embodiments, R^{1d} is hydrogen.

In embodiments, Ar is:

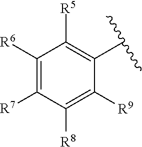

wherein R⁵, R⁶, R⁷, R⁸, and R⁹ are each independently selected from the group consisting of hydrogen, halo, alkyl, alkoxy, haloalkyl, trifluoromethyl, cycloalkyl and any two adjacent R⁵, R⁶, R⁷, R⁸, and R⁹ together combine to form a further fused ring that is an aromatic ring optionally comprising 1 to 3 heteroatoms independently selected from N, O or S, the further fused ring being optionally substituted.

In embodiments, there are provided compounds of Formula (VI) or pharmaceutically acceptable salt thereof:

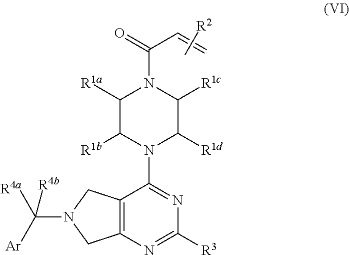

wherein:

Ar is selected from the group consisting of aryl, arylalkyl, arylcarbonyl, arylsulfonyl, heteroaryl, heteroarylalkyl, heteroaryl carbonyl, and heteroarylsulfonyl, any of which may be optionally substituted with halogens, hydroxyl, alkoxyl, alkyl, amidoalkyl;

R^{1a}, R^{1b}, R^{1c}, and R^{1d} are independently selected from hydrogen, cyano, alkyl, amido, alkylamido, CH₂SOₚMe, where p is 0 to 2, heteroarylalkyl, hydroxy alkyl, alkynylalkyl and cyanoalkyl; wherein any C—H present in R^{1a}, R^{1b}, R^{1c}, and R^{1d} is optionally exchanged for C—F; or any two R^{1a}, R^{1b}, R^{1c}, and R^{1d} combine to form to form a fused 3-6-membered ring or a 1 to 4 atom bridging unit, wherein each atom of the fused 3-6-membered ring or the 1 to 4 atom bridging unit comprises, independently, an optionally substituted methylene unit or a heteroatom selected from NR^{N}, O, or S, or SO₂, wherein R^{N} is hydrogen, alkyl, and fluorinated alkyl; and wherein the fused 3-6-membered ring or the 1 to 4 atom bridging unit is optionally substituted with oxo, halogen, and hydroxyl;

R² is selected from the group consisting of hydrogen, fluorine, methyl, and —CH₂NR^{a}R^{b}, wherein R^{a} and R^{b} are independently selected from hydrogen or alkyl; or R^{a} and R^{b} combine to form a C₂-C₆ nitrogen containing heterocycle;

R³ is selected from hydrogen, alkyl, alkoxy, amino, aminoalkylamino, halogen, heterocyclylalkoxy, aminoalkoxy, N-alkylaminoalkoxy, N,N-dialkylaminoalkoxy, mercapto-alkyl, mercapto aryl, aryl, any of which may be optionally substituted; and

R^{4a} and R^{4b} are independently selected from hydrogen, aryl, alkyl, trifluoroalkyl, alkyl optionally with halogen and cycloalkyl; or one of R^{4a} and R^{4b} forms a fused, non-aromatic ring structure with Ar.

or R^{4a} and R^{4b} together define a double-bonded oxygen (carbonyl).

In embodiments, R³ is O-linked N-methyl-L-prolinol.

In embodiments, R³ is hydrogen.

In embodiments, R² is hydrogen.

In embodiments, R² is fluorine.

In embodiments, R² is N,N-dimethylaminomethyl.

In embodiments, R^{1b} is methyl.

In embodiments, a stereogenic center created by the R^{1b} methyl group is in the R-configuration.

In embodiments, a stereogenic center created by the R^{1b} methyl group is in the S-configuration.

In embodiments, R^{1c} is methyl.

In embodiments, a stereogenic center created by the R^{1c} methyl group is in the R-configuration.

In embodiments, a stereogenic center created by the R^{1c} methyl group is in the S-configuration.

In embodiments, R^{1a} is cyanomethyl.

In embodiments, a stereogenic center created by the cyanomethyl group is in the R-configuration.

In embodiments, a stereogenic center created by the cyanomethyl group is in the S-configuration.

In embodiments, R^{1d} is hydrogen.

In embodiments, Ar is:

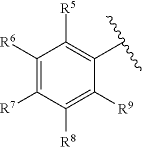

wherein R⁵, R⁶, R⁷, R⁸, and R⁹ are each independently selected from the group consisting of hydrogen, halo, alkyl, alkoxy, haloalkyl, trifluoromethyl, cycloalkyl and any two adjacent R⁵, R⁶, R⁷, R⁸, and R⁹ together combine to form a further fused ring that is an aromatic ring optionally comprising 1 to 3 heteroatoms independently selected from N, O or S, the further fused ring being optionally substituted.

In embodiments, Ar is selected from the group consisting of:

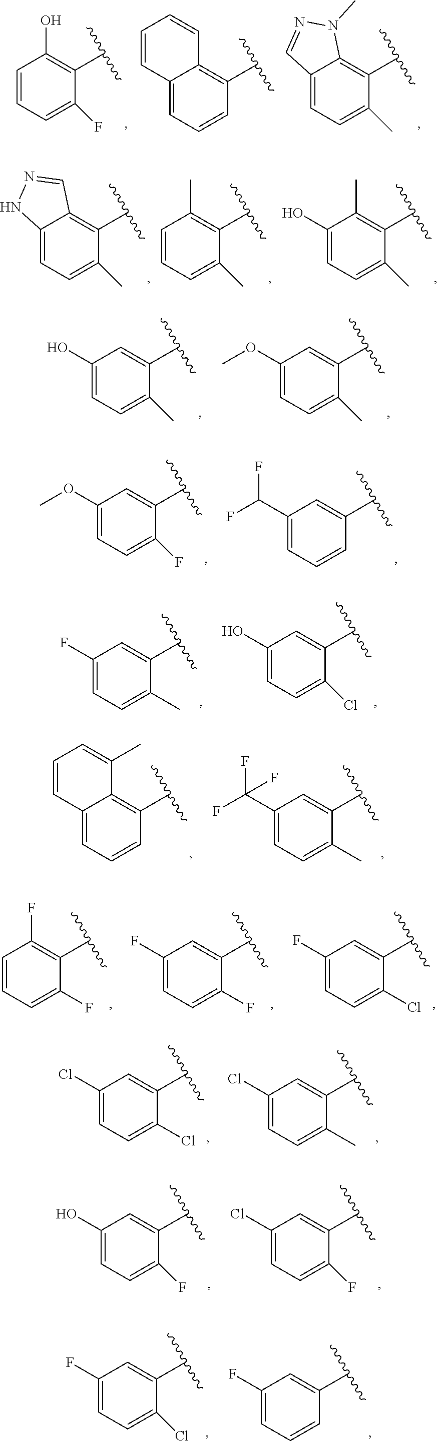

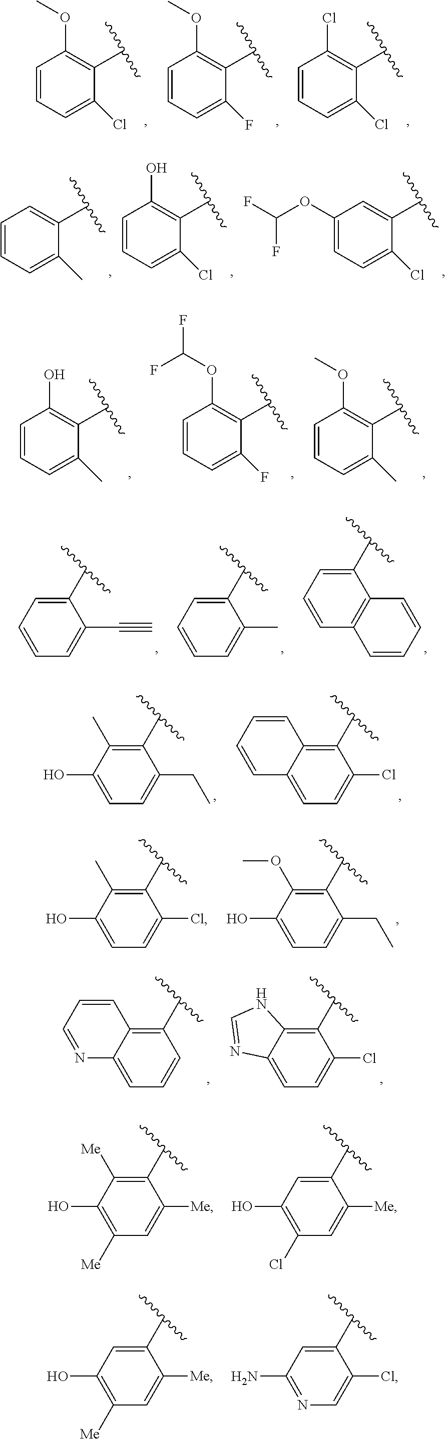

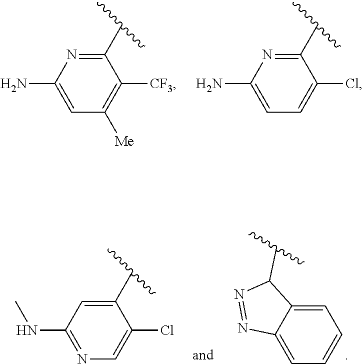

In embodiments, Ar is:

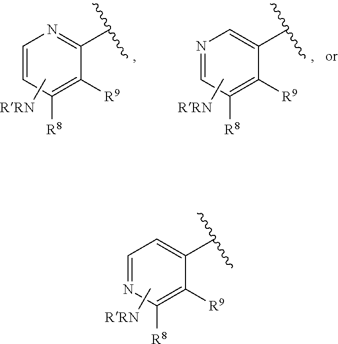

wherein R and R′ are independently hydrogen or C₁-C₄ alkyl; and R⁸, and R⁹ are each independently selected from the group consisting of hydrogen, halo, alkyl, alkoxy, haloalkyl, trifluoromethyl, cycloalkyl; or R⁸, and R⁹ together combine to form a further fused ring that is an aromatic ring optionally comprising 1 to 3 heteroatoms independently selected from N, O or S, the further fused ring being optionally substituted.

In embodiments, Ar is selected from the group consisting of:

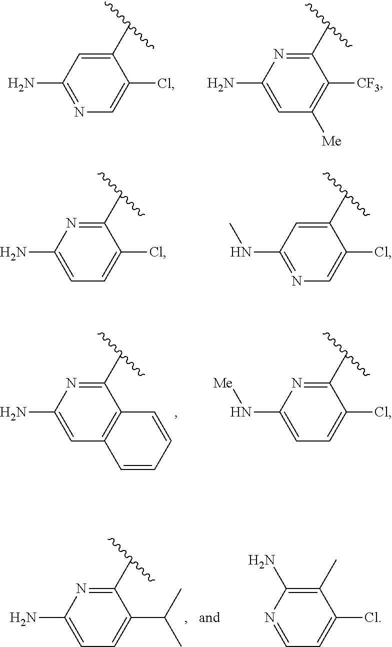

In embodiments, there are provided compound selected from Table 1, below.

In one or more of the preceding embodiments, R³ at the pyrimidine C-2 position is selected from:

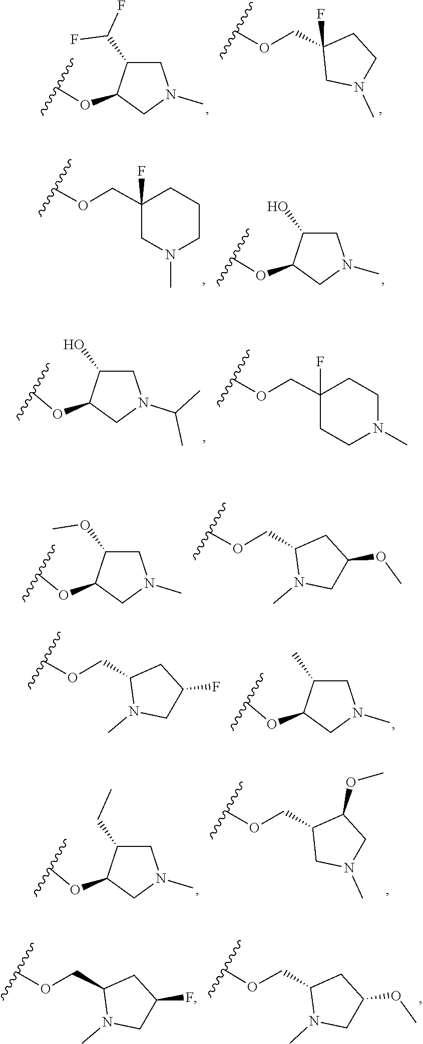

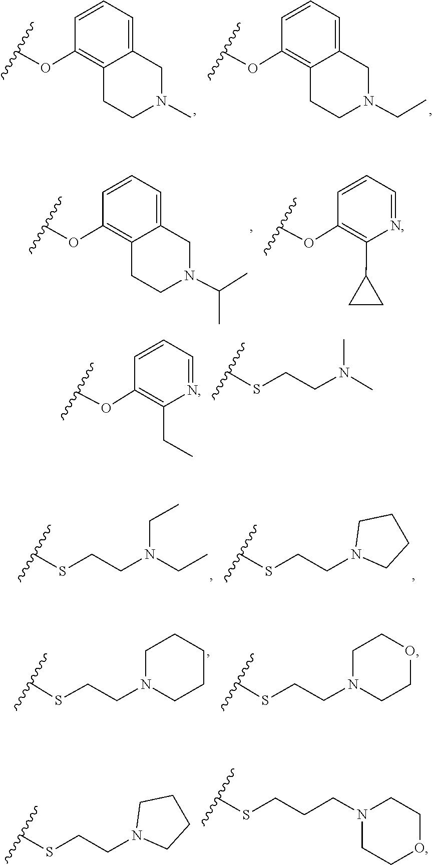

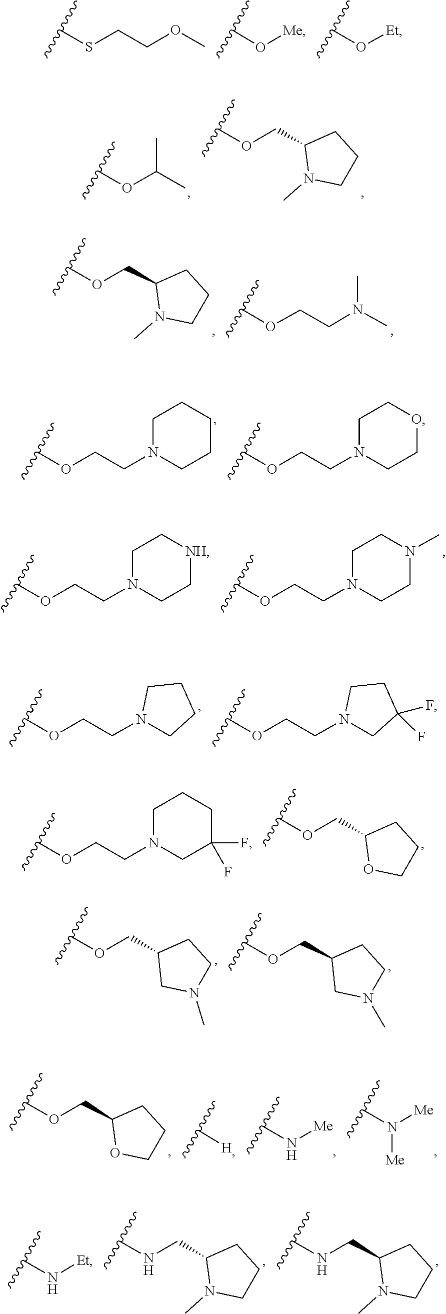

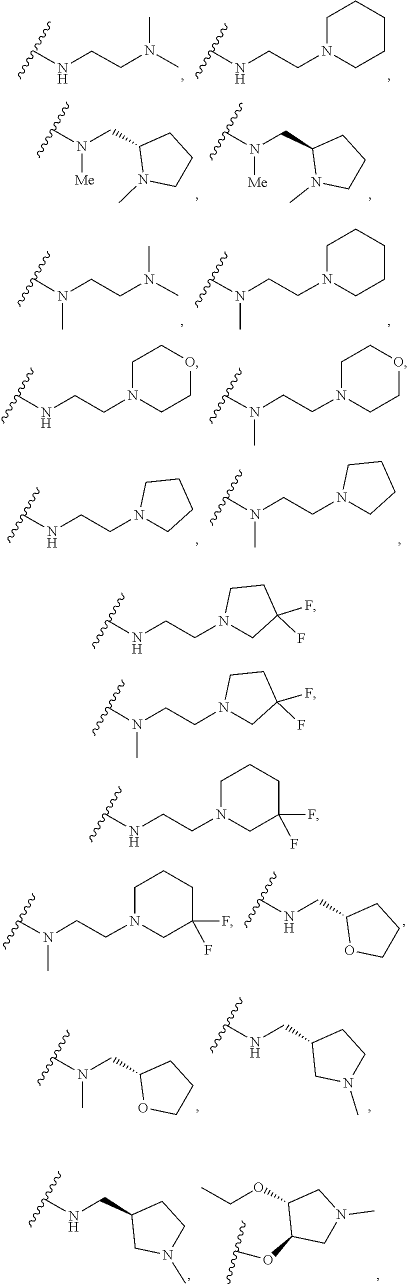

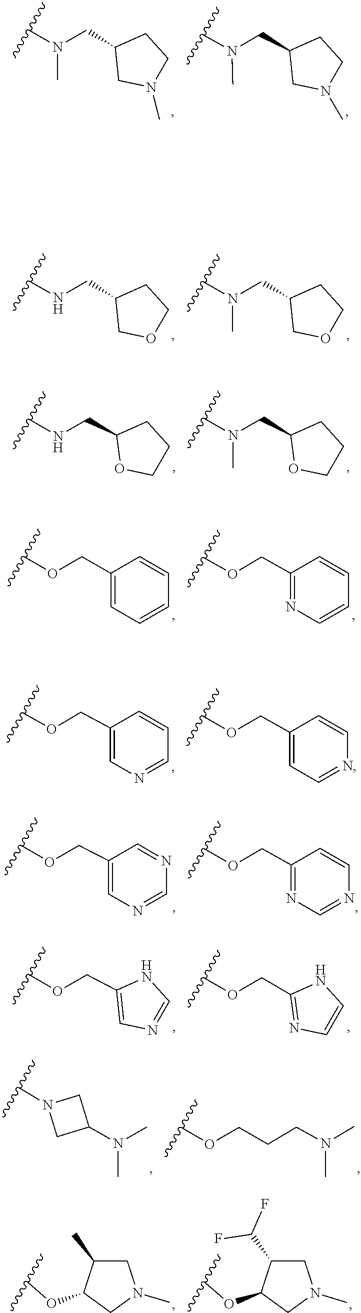

In one or more of the preceding embodiments, ArCR^{4a}R^{4b}-position is selected from:

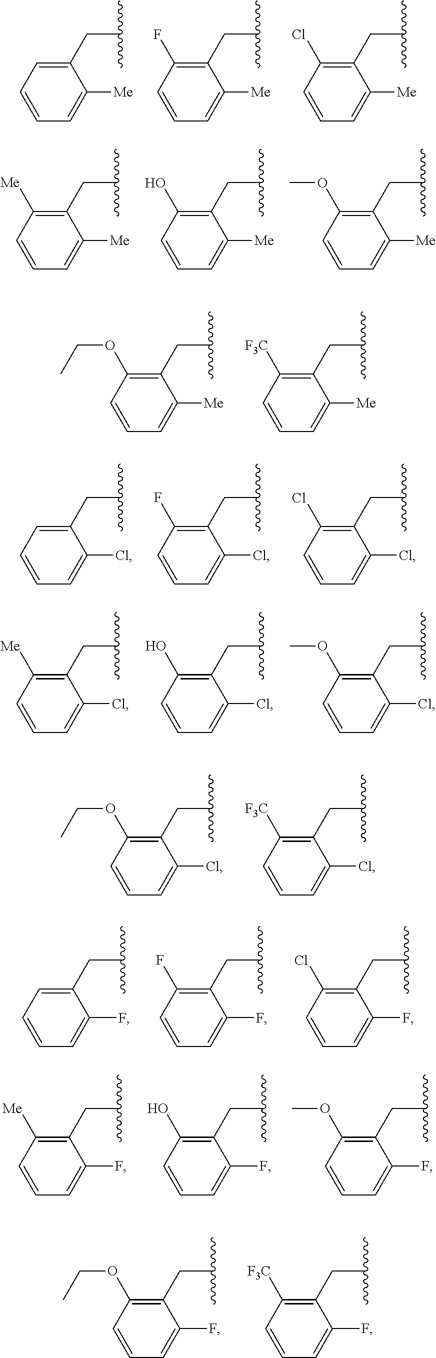

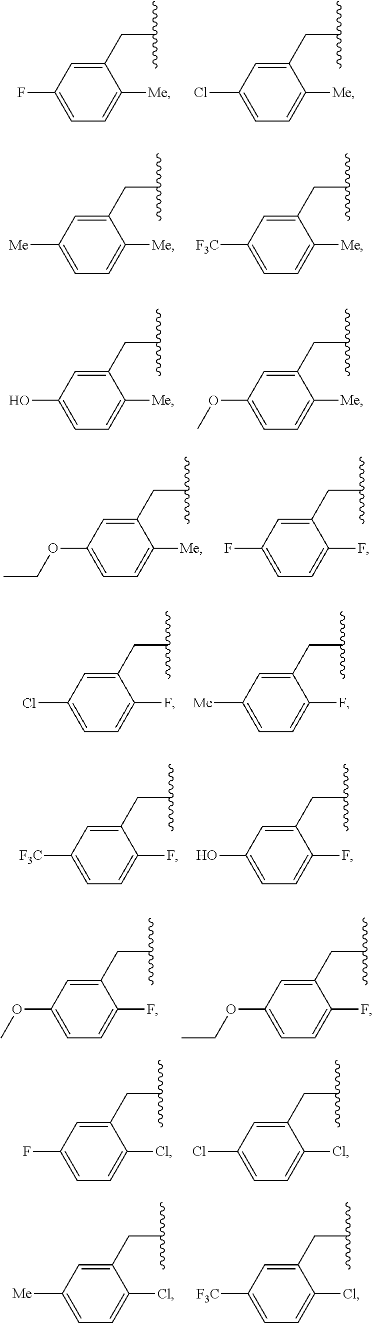

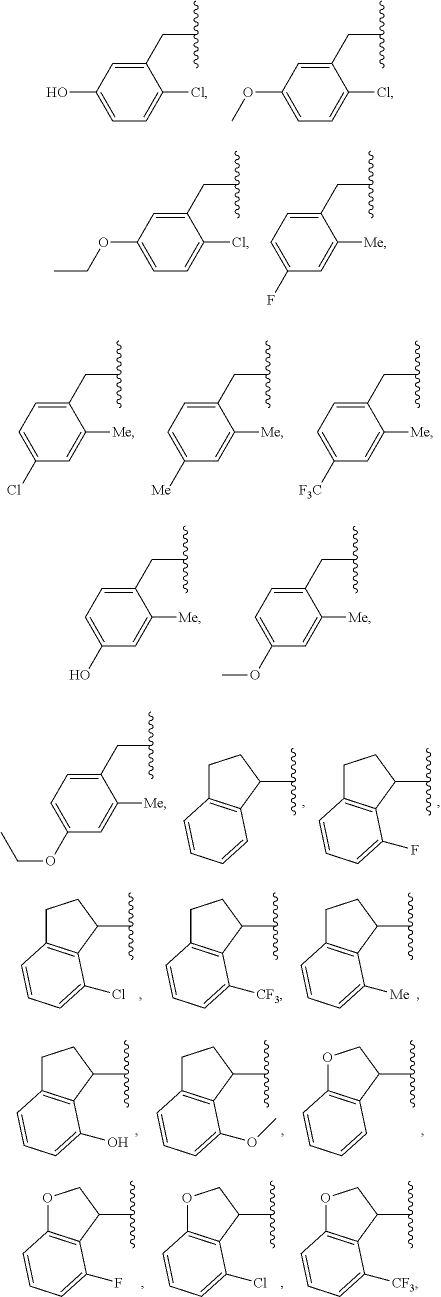

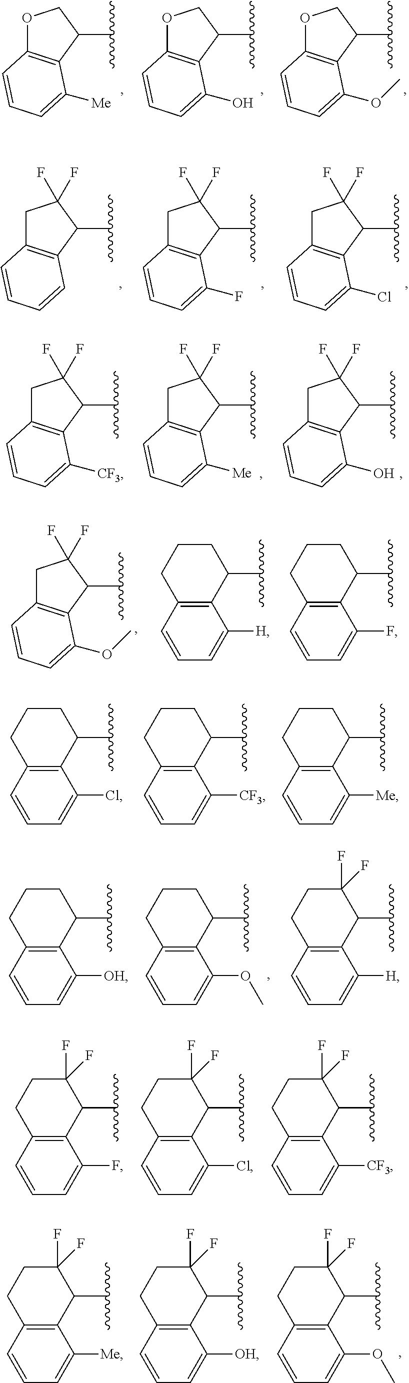

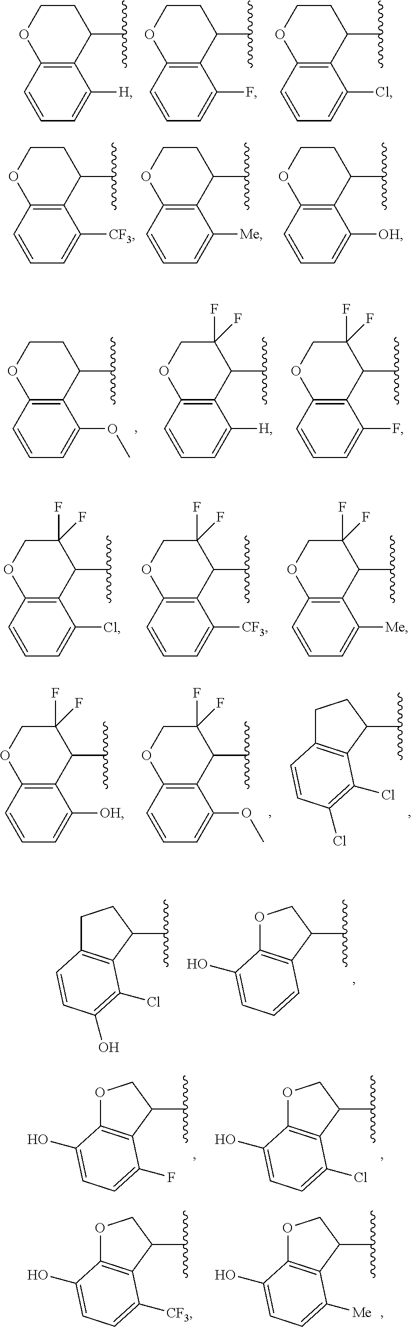

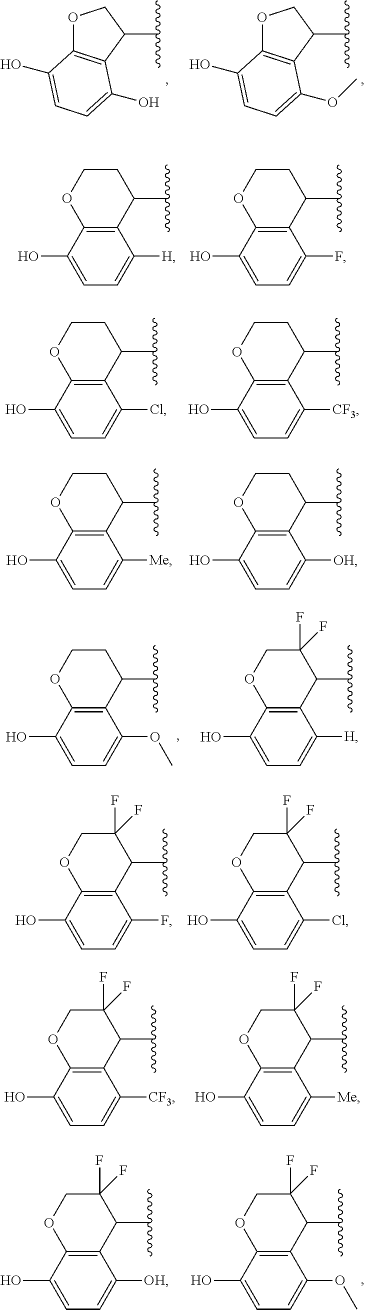

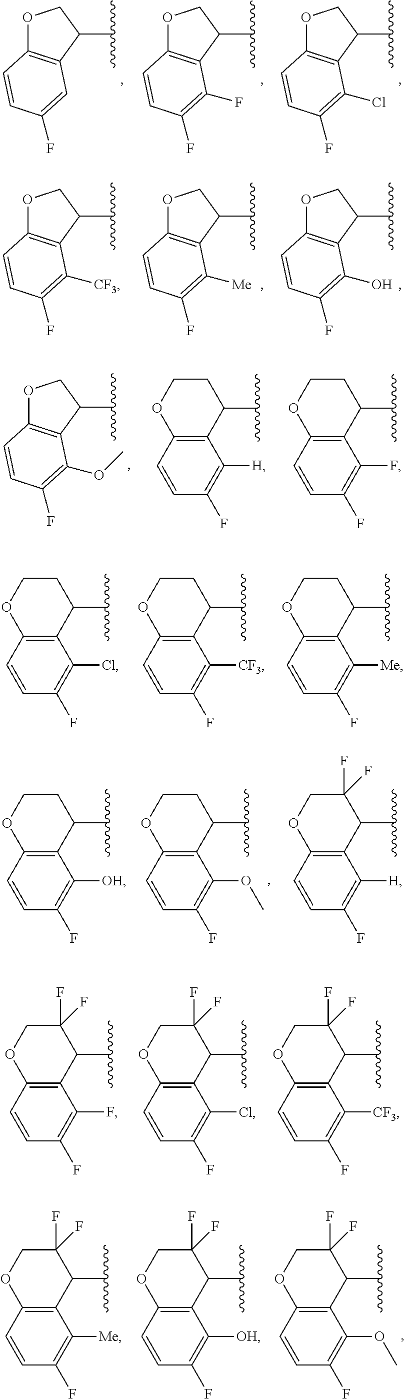

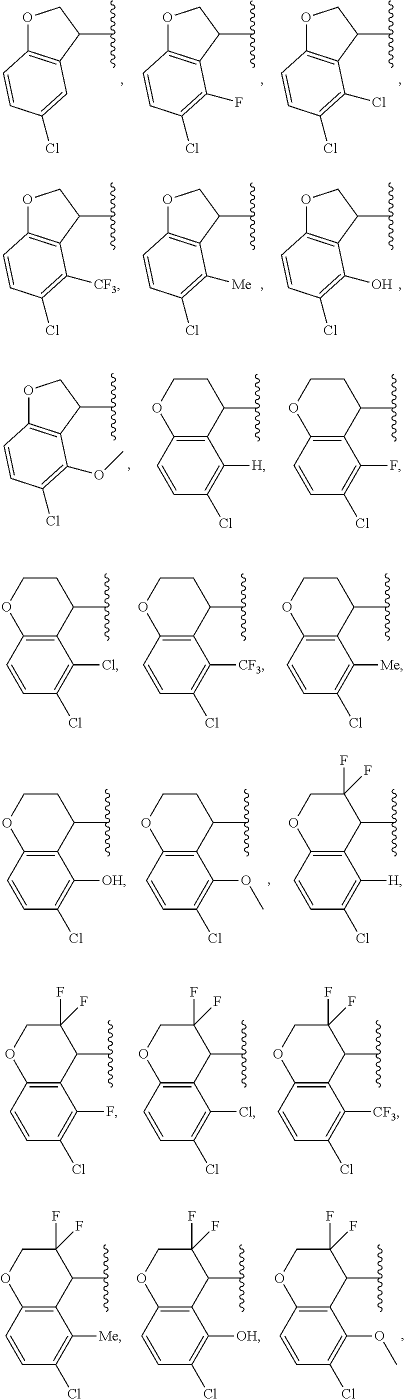

For each of the subgeneric structures disclosed hereinabove, electrophilic moiety substituted acrylamide bearing R² can alternatively be selected from:

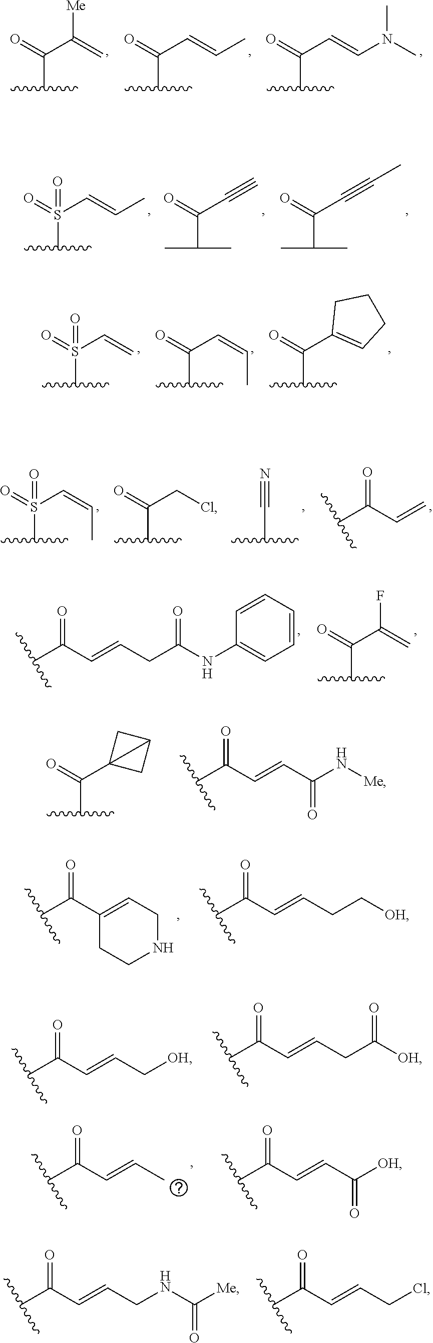

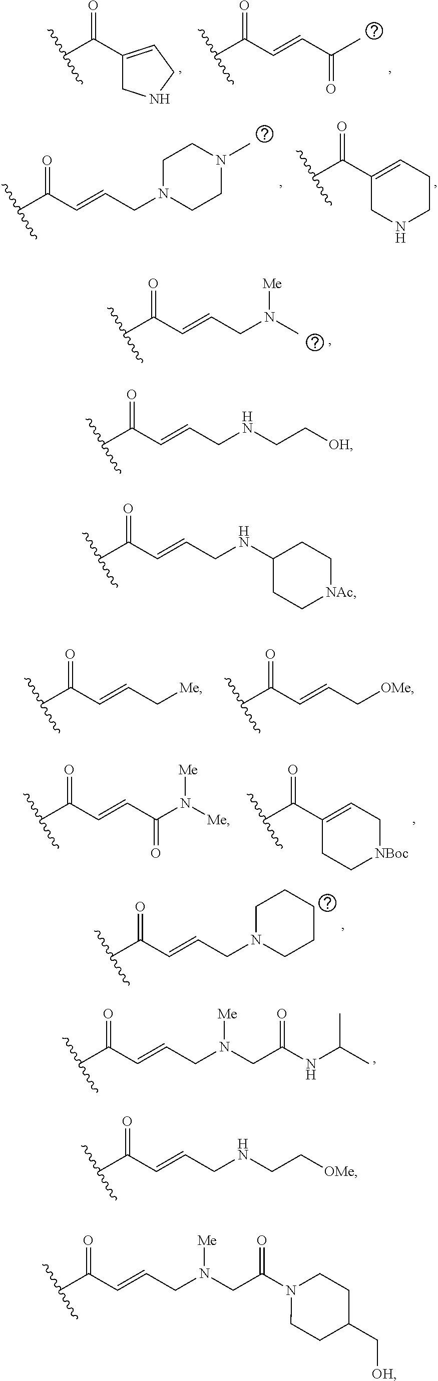

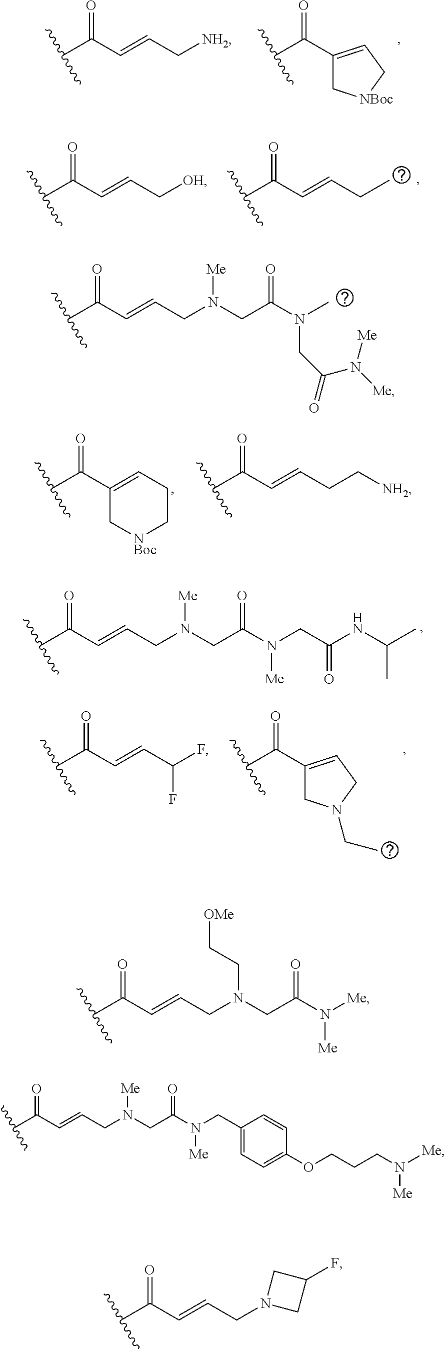

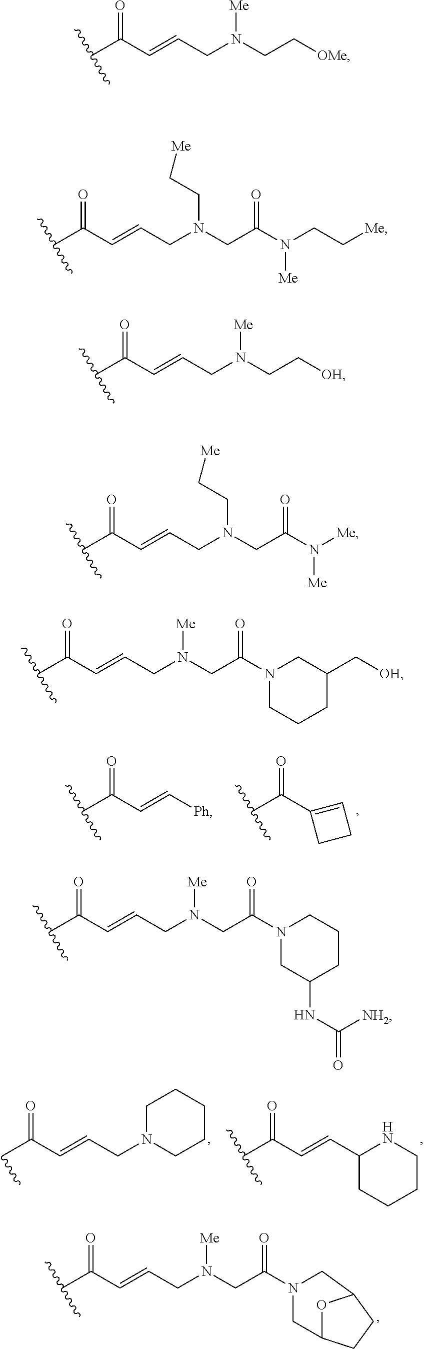

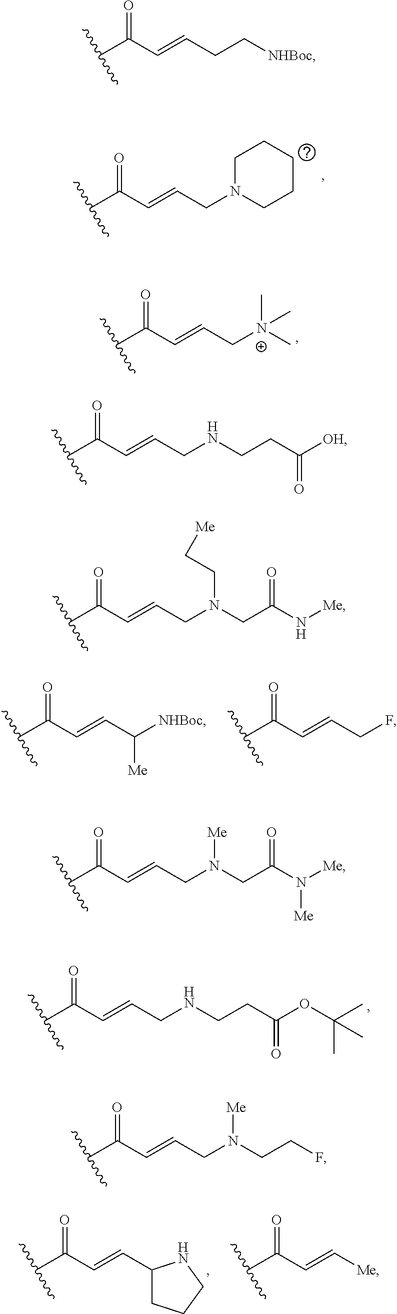

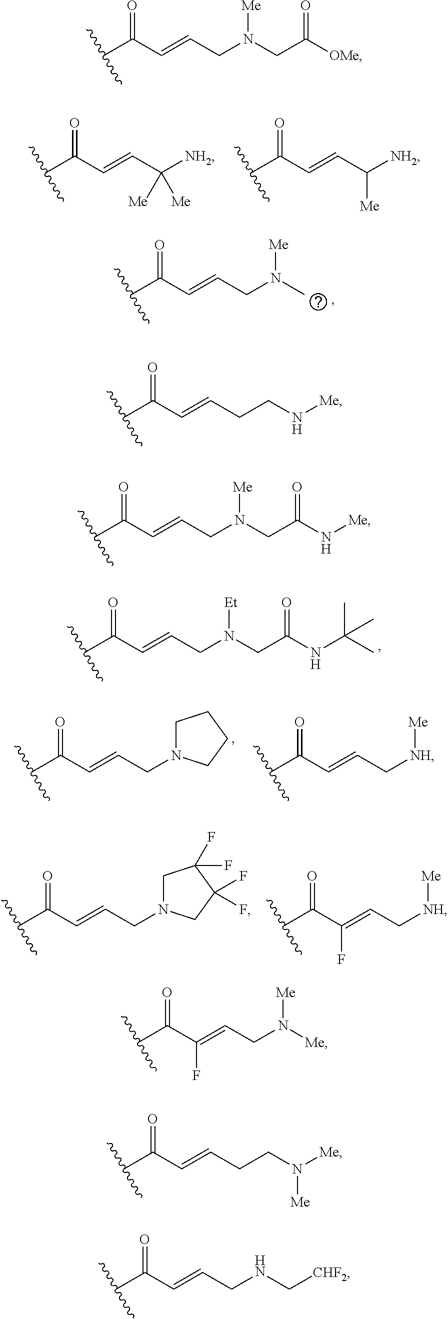

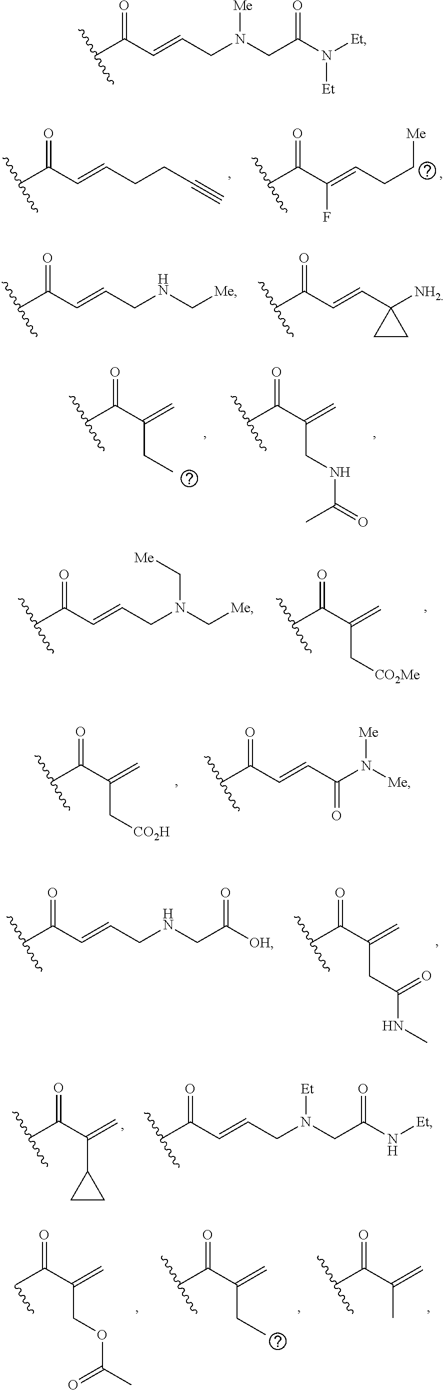

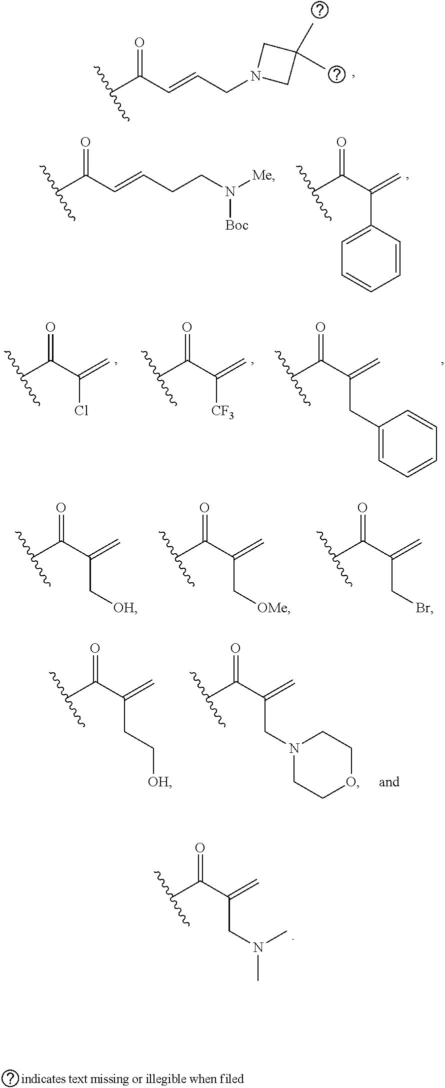

Embodiments disclosed herein are further illustrated by the following examples in Table 1.

TABLE 1

| | | | | CAF | |
|---|---|---|---|---|---|
| | | | | (10 | |
| | | | | min- | |
| | | | | utes/ | |
| | | | | 60 | |
| | | | | min- | |
| Exam- | | | | utes) | |
| ple | | | | at 10 | |
| Num- | | Proce- | | micro- | |
| ber | Structure | dure | [M + H] | molar | ¹H NMR |
| 1-1 | 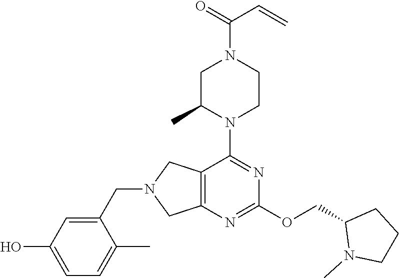 [SMILES: C=CC(=O)N1CCN(c2nc(OC[C@@H]3CCCN3C)nc3c2CN(Cc2cc(O)ccc2C)C3)[C@@H](C)C1] | A | 506.64 | 69%/ 93% | ¹H NMR (400 MHz, CD₃OD) δ 8.51 (s, 1H), 6.99 (d, J = 8.2 Hz, 1H), 6.83 (t, J = 6.7 Hz, 1H), 6.81-6.70 (m, 1H), 6.63 (dd, J = 8.1, 2.3 Hz, 1H), 6.28 (d, J = 16.6 Hz, 1H), 5.80 (d, J = 10.5 Hz, 1H), 4.68 (dd, J = 12.5, 2.8 Hz, 1H), 4.57 (s, 1H), 4.49 (d, J = 5.3 Hz, 1H), 4.38 (d, J = 13.5 Hz, 1H), 4.21-4.07 (m, 3H), 3.99 (d, J = 13.8 Hz, 1H), 3.91-3.79 (m, 3H), 3.76 (s, 2H), 3.71- 3.63 (m, 1H), 3.55 (d, J = 11.5 Hz, 1H), 3.40-3.34 (m, 1H), 3.20 (ddd, J = 24.7, 13.7, 5.0 Hz, 2H), 3.03 (s, 3H), 2.41-2.32 (m, 1H), 2.27 (s, 3H), 2.20-2.04 (m, 2H), 2.04-1.93 (m, 1H), 1.21 (d, J = 6.5 Hz, 3H). |
| 1-2 | 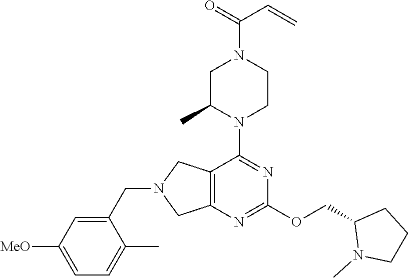 [SMILES: C=CC(=O)N1CCN(c2nc(OC[C@@H]3CCCN3C)nc3c2CN(Cc2cc(OC)ccc2C)C3)[C@@H](C)C1] | A | 520.67 | 0%/ 4% | Intermediate. No NMR. |
| 1-3 | 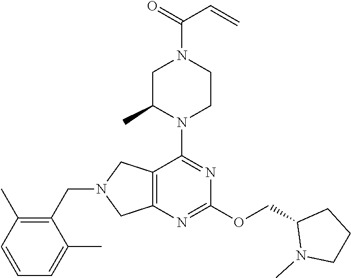 [SMILES: C=CC(=O)N1CCN(c2nc(OC[C@@H]3CCCN3C)nc3c2CN(Cc2c(C)cccc2C)C3)[C@@H](C)C1] | A | 504.67 | 6%/ 33% | 1H NMR (400 MHz, CD₃OD) δ 7.10-6.99 (m, 3H), 6.80 (td, J = 17.1, 10.4 Hz, 1H), 6.28 (dd, J = 17.1, 4.8 Hz, 1H), 5.89-5.74 (m, 1H), 4.75-4.00 (m, 8H), 3.97 (s, 2H), 3.77-3.48 (m, 5H), 3.21- 3.01 (m, 2H), 2.97 (s, 3H), 2.43 (s, 6H), 2.38-1.88 (m, 5H), 1.20 (d, J = 6.6 Hz, 3H). |
| 1-6 | 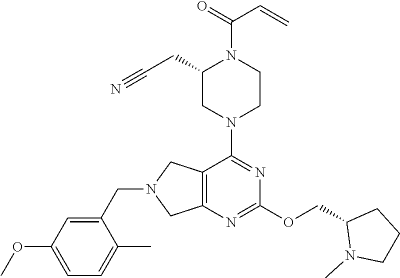 [SMILES: C=CC(=O)N1CCN(c2nc(OC[C@@H]3CCCN3C)nc3c2CN(Cc2cc(OC)ccc2C)C3)C[C@@H]1CC#N] | A | 546.3 | 65%/ 93% | 1H NMR (400 MHz, CD₃OD) δ 7.08 (d, J = 8.3 Hz, 1H), 6.94 (d, J = 2.7 Hz, 1H), 6.74 (dd, J = 8.3, 2.7 Hz, 2H), 6.27 (d, J = 16.3 Hz, 1H), 5.81 (d, J = 9.6 Hz, 1H), 4.97 (s, 1H), 4.62 (d, J = 13.5 Hz, 1H), 4.41-4.29 (m, 2H), 4.12 (s, 3H), 3.88 (s, 2H), 3.76 (s, 3H), 3.73 (s, 2H), 3.50 (d, J = 19.5 Hz, 1H), 3.14 (d, J = 4.5 Hz, 2H), 2.89 (dd, J = 16.6, 7.7 Hz, 3H), 2.56 (s, 3H), 2.46 (d, J = 8.0 Hz, 1H), 2.30 (s, 3H), 2.11 (dt, J = 21.0, 8.3 Hz, 2H), 1.95-1.78 (m, 2H), 1.74 (dd, J = 13.3, 6.4 Hz, 1H). |
| 1-7 | 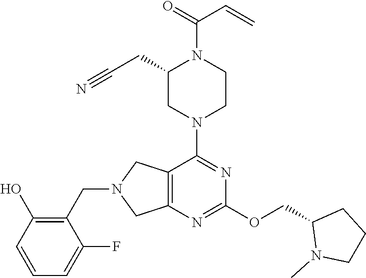 [SMILES: C=CC(=O)N1CCN(c2nc(OC[C@@H]3CCCN3C)nc3c2CN(Cc2c(O)cccc2F)C3)C[C@@H]1CC#N] | B | 536.3 | 96%/ 97% | 1H NMR (400 MHz, CD₃OD) δ 7.19-7.08 (m, 1H), 6.77 (s, 1H), 6.70-6.52 (m, 2H), 6.27 (d, J = 16.2 Hz, 1H), 5.82 (s, 1H), 4.98 (s, 1H), 4.63 (dd, J = 54.8, 41.8 Hz, 2H), 4.35 (d, J = 5.8 Hz, 2H), 4.20 (s, 2H), 4.10 (t, J = 13.4 Hz, 3H), 3.80 (s, 2H), 3.54 (s, 1H), 3.35 (s, 1H), 3.13 (dd, J = 9.5, 4.8 Hz, 2H), 2.87 (d, J = 4.7 Hz, 3H), 2.55 (d, J = 4.5 Hz, 3H), 2.45 (d, J = 9.0 Hz, 1H), 2.15-2.03 (m, 1H), 1.90-1.78 (m, 2H), 1.76-1.64 (m, 1H). |
| 1-8 | 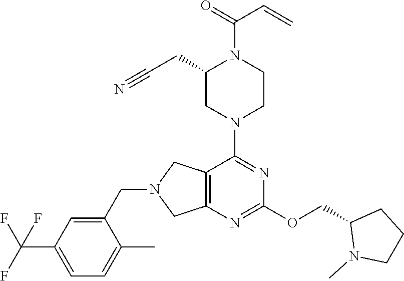 [SMILES: C=CC(=O)N1CCN(c2nc(OC[C@@H]3CCCN3C)nc3c2CN(Cc2cc(C(F)(F)F)ccc2C)C3)C[C@@H]1CC#N] | A | 584.3 | 20%/ 62% | 1H NMR (400 MHz, CD₃OD) δ 8.44 (s, 2H), 7.68 (s, 1H), 7.48 (d, J = 7.9 Hz, 1H), 7.38 (d, J = 7.9 Hz, 1H), 6.79 (d, J = 12.1 Hz, 1H), 6.27 (d, J = 16.5 Hz, 1H), 5.82 (d, J = 10.0 Hz, 1H), 5.04-4.92 (m, 1H), 4.76 (d, J = 11.7 Hz, 2H), 4.52 (dd, J = 12.5, 7.5 Hz, 1H), 4.19 (s, 2H), 4.16-4.03 (m, 1H), 4.01 (s, 2H), 3.83 (d, J = 5.6 Hz, 1H), 3.75 (s, 2H), 3.67 (dd, J = 12.0, 6.8 Hz, 1H), 3.53 (s, 1H), 3.45-3.31 (m, 2H), 3.19 (dt, J = 8.2, 6.1 Hz, 2H), 3.02 (s, 3H), 2.93 (d, J = 7.6 Hz, 2H), 2.47 (s, 3H), 2.41-2.28 (m, 1H), 2.24-1.90 (m, 3H). |
| 1-9 | 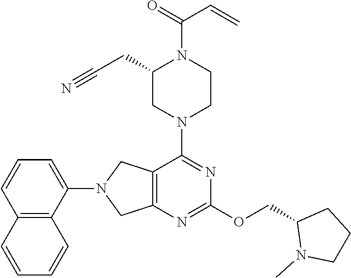 [SMILES: C=CC(=O)N1CCN(c2nc(OC[C@@H]3CCCN3C)nc3c2CN(c2cccc4ccccc24)C3)C[C@@H]1CC#N] | C | 538.3 | 35%/ 85% | 1H NMR (400 MHz, CD₃OD) δ 8.51 (s, 1H), 8.26 (dd, J = 6.0, 3.7 Hz, 1H), 7.94-7.79 (m, 1H), 7.60 (d, J = 8.0 Hz, 1H), 7.54-7.32 (m, 4H), 6.78 (d, J = 11.7 Hz, 1H), 6.28 (d, J = 16.7 Hz, 1H), 5.82 (d, J = 9.6 Hz, 1H), 4.98 (s, 1H), 4.85 (s, 2H), 4.76 (s, 2H), 4.56-4.46 (m, 3H), 4.10 (dd, J = 43.6, 11.5 Hz, 2H), 3.71 (s, 1H), 3.65- 3.58 (m, 1H), 3.41 (s, 2H), 3.11 (dd, J = 19.0, 8.1 Hz, 2H), 2.97 (d, J = 16.8 Hz, 4H), 2.82 (s, 1H), 2.36 (dd, J = 12.6, 6.3 Hz, 1H), 2.19-1.94 (m, 3H). |
| 1-11 | 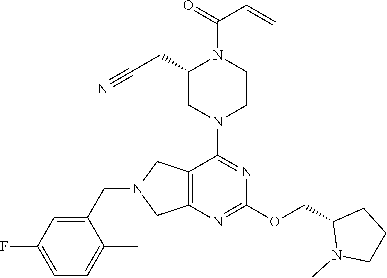 [SMILES: C=CC(=O)N1CCN(c2nc(OC[C@@H]3CCCN3C)nc3c2CN(Cc2cc(F)ccc2C)C3)C[C@@H]1CC#N] | A | 534.3 | 87%/ 98% | 1H NMR (400 MHz, CD₃OD) δ 7.26-7.25 (m, 2H), 6.88-6.77 (m, 2H), 6.29 (d, 1H), 5.82 (d, 1H), 4.58 (d, 2H), 4.33-4.32 (m, 2H) 4.13-4.11 (m, 4H), 3.93 (s, 2H), 3.75 (s, 2H), 3.75-3.74 (m, 1H), 3.12- 3.09 (m, 2H), 3.08-2.90 (m, 3H), 2.50 (s, 3H), 2.34- 2.32 (m, 1H), 2.09-2.07 (m, 1H), 1.82-1.67 (m, 3H). |
| 1-12 | 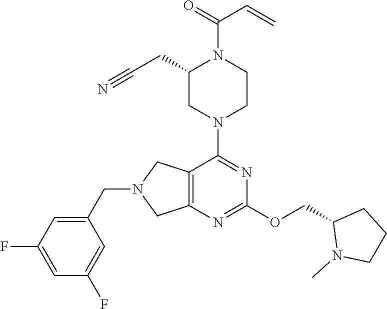 [SMILES: C=CC(=O)N1CCN(c2nc(OC[C@@H]3CCCN3C)nc3c2CN(Cc2cc(F)cc(F)c2)C3)C[C@@H]1CC#N] | B | 538.3 | 28%/ 74% | 1H NMR (400 MHz, CDCl₃) δ 6.91 (d, J = 6.5 Hz, 2H), 6.71 (t, J = 8.9 Hz, 1H), 6.63-6.46 (m, 1H), 6.34 (dd, J = 16.8, 1.0 Hz, 1H), 5.74 (d, J = 10.4 Hz, 1H), 4.61 (d, J = 13.4 Hz, 1H), 4.41 (d, J = 12.3 Hz, 2H), 4.19 (s, 1H), 3.99 (q, J = 11.0 Hz, 3H), 3.91- 3.83 (m, 2H), 3.80 (d, J = 14.6 Hz, 3H), 3.43 (d, J = 12.0 Hz, 1H), 3.24 (d, J = 12.8 Hz, 2H), 3.07 (d, J = 12.1 Hz, 1H), 2.83 (s, 1H), 2.56 (s, 3H), 2.39 (s, 1H), 2.08 (s, 1H), 1.84 (d, J = 34.0 Hz, 4H), 1.17 (d, J = 6.7 Hz, 3H). |
| 1-13 | 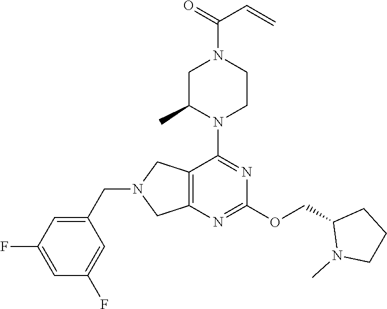 [SMILES: C=CC(=O)N1CCN(c2nc(OC[C@@H]3CCCN3C)nc3c2CN(Cc2cc(F)cc(F)c2)C3)[C@@H](C)C1] | A | 513.7 | 0%/ 0% | 1H NMR (400 MHz, CD₃OD) δ 7.44-7.34 (m, 1H), 7.10-6.97 (m, 2H), 6.86- 6.73 (m, 1H), 6.28 (dd, J = 16.6, 3.3 Hz, 1H), 5.80 (dd, J = 10.6, 1.7 Hz, 1H), 4.61 (s, 1H), 4.52-4.37 (m, 1H), 4.33-4.25 (m, 2H), 4.17 (t, J = 8.1 Hz, 3H), 4.03 (s, 2H), 3.75 (s, 2H), 3.67-3.52 (m, 1H), 3.39 (t, J = 10.5 Hz, 2H), 3.19-3.00 (m, 2H), 2.81- 2.72 (m, 1H), 2.50 (s, 3H), 2.38 (dd, J = 17.9, 9.0 Hz, 1H), 2.07 (dt, J = 12.6, 6.1 Hz, 1H), 1.82 (dq, J = 9.3, 5.4 Hz, 2H), 1.68 (dd, J = 13.0, 6.5 Hz, 1H), 1.21 (d, J = 6.3 Hz, 3H). |
| 1-14 | 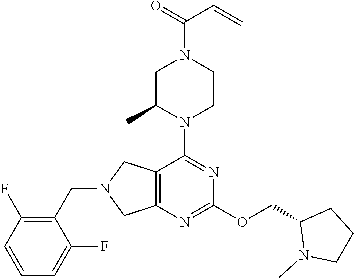 [SMILES: C=CC(=O)N1CCN(c2nc(OC[C@@H]3CCCN3C)nc3c2CN(Cc2c(F)cccc2F)C3)[C@@H](C)C1] | A | 513.3 | 0%/ 8% | 1H NMR (400 MHz, CD₃OD) δ 7.67 (s, 1H), 7.48 (d, J = 8.3 Hz, 1H), 7.37 (d, J = 7.9 Hz, 1H), 6.86-6.73 (m, 1H), 6.27 (d, J = 16.5 Hz, 1H), 5.79 (dd, J = 10.6, 1.9 Hz, 1H), 4.61 (s, 1H), 4.51-4.36 (m, 1H), 4.30 (qd, J = 11.0, 5.9 Hz, 2H), 4.21 (s, 1H), 4.12 (s, 2H), 3.98 (s, 2H), 3.70 (s, 2H), 3.54 (d, J = 9.9 Hz, 1H), 3.36 (d, J = 9.3 Hz, 1H), 3.15 (d, J = 13.5 Hz, 1H), 3.08 (dd, J = 9.6, 4.8 Hz, 1H), 3.05-2.93 (m, 1H), 2.75 (s, 1H), 2.50 (s, 3H), 2.47 (s, 3H), 2.36 (dd, J = 17.9, 9.2 Hz, 1H), 2.08 (ddd, J = 16.9, 12.7, 8.7 Hz, 1H), 1.85-1.77 (m, 2H), 1.68 (dt, J = 13.7, 7.1 Hz, 1H), 1.20 (d, J = 6.6 Hz, 3H). |
| 1-15 | 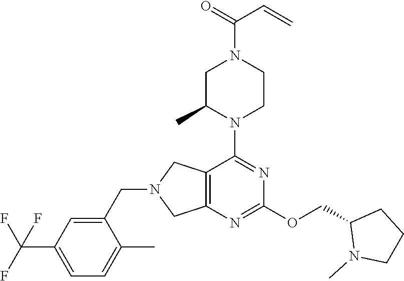 [SMILES: C=CC(=O)N1CCN(c2nc(OC[C@@H]3CCCN3C)nc3c2CN(Cc2cc(C(F)(F)F)ccc2C)C3)[C@@H](C)C1] | A | 559.3 | 0%/ 0% | 1H NMR (400 MHz, CD₃OD) δ 7.88-7.82 (m, 2H), 7.70-7.68 (m, 1H), 7.39-7.30 (m, 3H), 6.75 (br, 1H), 6.28 (d, 1H), 5.81 (d, 1H), 4.95-4.94 (m, 1H), 4.50- 4.06 (m, 10H), 3.60-3.52 (m, 3H), 3.48-3.46 (m, 1H), 3.12 (s, 4H), 2.83-2.70 (m, 3H), 2.51 (s, 3H), 2.49- 2.45 (m, 1H), 2.01-2.00 (m, 1H), 1.72-1.62 (m, 3H). |
| 1-16 | 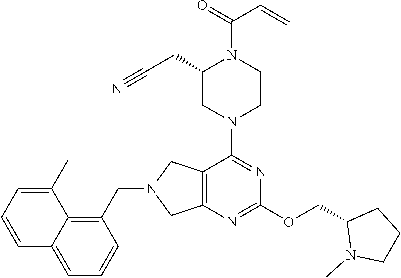 [SMILES: C=CC(=O)N1CCN(c2nc(OC[C@@H]3CCCN3C)nc3c2CN(Cc2cccc4cccc(C)c24)C3)C[C@@H]1CC#N] | B | 566.3 | 40%/ 86% | 1H NMR (400 MHz, CD₃OD) δ 7.70-7.60 (m, 2H), 7.46-7.36 (m, 1H), 7.32- 7.34 (m, 1H), 7.27-7.25 (m, 2H), 6.75-6.66 (m, 1H), 6.28 (d, 1H), 5.81 (d, 1H), 4.87-4.86 (m, 1H), 4.39- 4.19 (m, 10H), 3.31-3.30 (m, 1H), 3.29-3.26 (m, 2H), 2.81-2.80 (m, 4H), 2.79-2.75 (m, 2H), 2.56 (s, 3H), 2.37- 2.36 (m, 1H), 2.10-2.08 (m, 1H), 1.86-1.70 (m, 3H). |
| 1-17 | 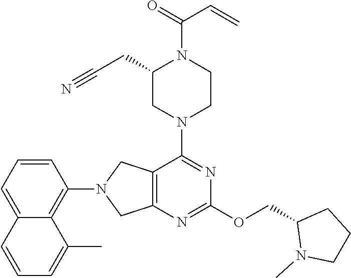 [SMILES: C=CC(=O)N1CCN(c2nc(OC[C@@H]3CCCN3C)nc3c2CN(c2cccc4cccc(C)c24)C3)C[C@@H]1CC#N] | P | 552.3 | 90%/ 99% | 1H NMR (400 MHz, CDCl₃) δ 8.35 (s, 1H), 7.03- 6.97 (m, 1H), 6.87 (s, 1H), 6.68 (dd, J = 8.1, 2.6 Hz, 1H), 6.64-6.47 (m, 1H), 6.34 (ddd, J = 16.8, 11.4, 1.7 Hz, 1H), 5.78-5.72 (m, 1H), 5.22 (s, 3H), 4.95 (s, 1H), 4.77 (dd, J = 28.0, 20.2 Hz, 2H), 4.53-4.46 (m, 1H), 4.37 (d, J = 13.9 Hz, 1H), 4.26 (s, 1H), 4.08 (d, J = 5.7 Hz, 1H), 4.05- 3.97 (m, 1H), 3.82 (d, J = 6.8 Hz, 4H), 3.72 (s, 1H), 3.65-3.58 (m, 1H), 3.55 (d, J = 10.6 Hz, 1H), 3.38 (t, J = 12.5 Hz, 1H), 3.17 (dd, J = 13.7, 4.2 Hz, 1H), 2.90 (s, 3H), 2.30-2.19 (m, 5H), 2.11-2.01 (m, 2H), 1.33-1.13 (m, 6H). |
| 1-18 | 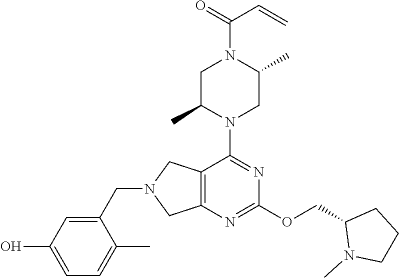 [SMILES: C=CC(=O)N1C[C@H](C)N(c2nc(OC[C@@H]3CCCN3C)nc3c2CN(Cc2cc(O)ccc2C)C3)C[C@H]1C] | A | 521.7 | 21%/ 65% | 1H NMR (400 MHz, CDCl₃) δ 7.16 (d, J = 8.6 Hz, 1H), 6.98 (t, J = 2.3 Hz, 1H), 6.72 (dd, J = 8.6, 2.9 Hz, 1H), 6.55 (ddd, J = 42.0, 16.7, 10.6 Hz, 1H), 6.34 (ddd, J = 16.8, 12.1, 1.7 Hz, 1H), 5.76 (dd, J = 15.2, 6.1 Hz, 1H), 5.02-4.84 (m, 1H), 4.69 (t, J = 4.4 Hz, 1H), 4.49-4.27 (m, 2H), 4.25-4.12 (m, 2H), 4.10 (d, J = 6.6 Hz, 1H), 4.01 (dd, J = 11.9, 4.5 Hz, 1H), 3.95-3.85 (m, 2H), 3.82 (s, 2H), 3.58 (d, J = 3.2 Hz, 1H), 3.36 (dd, J = 21.4, 8.0 Hz, 1H), 3.15 (d, J = 9.8 Hz, 1H), 2.74 (dt, J = 12.4, 6.0 Hz, 1H), 2.51 (s, 3H), 2.37-2.30 (m, 1H), 2.08- 2.00 (m, 1H), 1.88-1.71 (m, 3H), 1.26 (d, J = 6.5 Hz, 3H), 1.19-1.17 (m, 3H). |
| 1-19 | 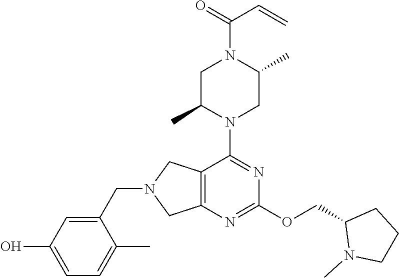 [SMILES: C=CC(=O)N1C[C@H](C)N(c2nc(OC[C@@H]3CCCN3C)nc3c2CN(Cc2cc(O)ccc2C)C3)C[C@H]1C] | A | 541.3 | 47%/ 76% | 1H NMR (400 MHz, CD₃OD) δ 7.15 (ddd, J = 12.6, 9.1, 4.3 Hz, 2H), 6.90 (td, J = 8.5, 2.7 Hz, 1H), 6.85- 6.71 (m, 1H), 6.27 (dd, J = 16.7, 3.3 Hz, 1H), 5.79 (dd, J = 10.6, 1.7 Hz, 1H), 4.62 (s, 1H), 4.43 (dd, J = 40.7, 13.3 Hz, 1H), 4.30 (dt, J = 10.9, 5.1 Hz, 2H), 4.21-4.14 (m, 1H), 4.14-4.08 (m, 2H), 4.07-3.96 (m, 1H), 3.89 (s, 2H), 3.71 (s, 2H), 3.54 (d, J = 11.1 Hz, 1H), 3.36 (d, J = 9.2 Hz, 1H), 3.18-2.97 (m, 2H), 2.74 (dt, J = 13.6, 6.7 Hz, 1H), 2.49 (s, 3H), 2.39-2.30 (m, 4H), 2.08 (ddd, J = 16.3, 12.5, 8.2 Hz, 1H), 1.85-1.76 (m, 2H), 1.68 (dt, J = 19.8, 7.3 Hz, 1H), 1.20 (d, J = 6.5 Hz, 3H). |
| 1-20 | 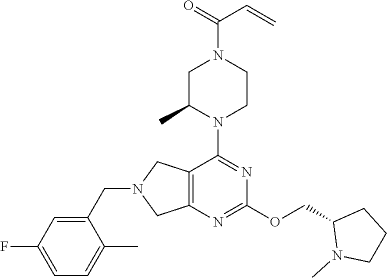 [SMILES: C=CC(=O)N1CCN(c2nc(OC[C@@H]3CCCN3C)nc3c2CN(Cc2cc(F)ccc2C)C3)[C@@H](C)C1] | A | 509.3 | 0%/ 6% | 1H NMR (400 MHz, DMSO) δ 8.98 (s, 1H), 6.88- 6.73 (m, 2H), 6.64 (d, J = 8.1 Hz, 1H), 6.15 (dd, J = 16.6, 2.5 Hz, 1H), 5.76- 5.66 (m, 1H), 4.73 (s, 1H), 4.44 (d, J = 30.4 Hz, 1H), 4.23 (dd, J = 10.7, 4.8 Hz, 1H), 4.15 (d, J = 13.7 Hz, 1H), 4.03 (dd, J = 17.8, 9.0 Hz, 2H), 3.93 (d, J = 11.1 Hz, 1H), 3.80 (d, J = 9.6 Hz, 3H), 3.62-3.46 (m, 3H), 3.27 (s, 1H), 3.13 (dd, J = 13.6, 3.7 Hz, 1H), 2.94 (d, J = 4.0 Hz, 1H), 2.32 (s, 3H), 2.26 (s, 3H), 2.18 (s, 3H), 2.13 (d, J = 8.7 Hz, 1H), 1.94-1.85 (m, 1H), 1.69-1.61 (m, 2H), 1.60-1.51 (m, 1H), 1.17- 1.06 (m, 6H). |
| 1-21 | 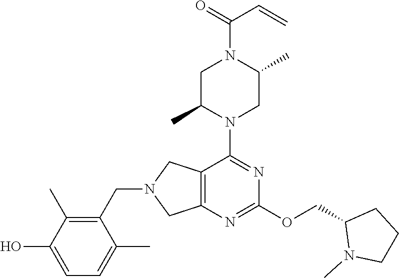 [SMILES: C=CC(=O)N1C[C@H](C)N(c2nc(OC[C@@H]3CCCN3C)nc3c2CN(Cc2c(C)ccc(O)c2C)C3)C[C@H]1C] | A | 535.3 | 20%/ 63% | 1H NMR (400 MHz, CDCl₃) δ 9.14 (s, 2H), 8.40 (s, 1H), 8.22 (s, 1H), 7.31 (d, J = 8.4 Hz, 1H), 7.17 (d, J = 8.4 Hz, 1H), 6.50 (ddd, J = 40.8, 16.7, 10.5 Hz, 1H), 6.28 (dd, J = 15.9, 12.0 Hz, 1H), 5.69 (t, J = 9.4 Hz, 1H), 4.87 (s, 1H), 4.65 (d, J = 5.3 Hz, 1H), 4.44 (d, J = 11.7 Hz, 1H), 4.30 (d, J = 13.8 Hz, 1H), 4.16 (s, 2H), 3.98 (dt, J = 18.8, 10.1 Hz, 3H), 3.76 (s, 2H), 3.61 (d, J = 24.0 Hz, 2H), 3.49 (dd, J = 28.5, 11.1 Hz, 2H), 3.30 (t, J = 11.8 Hz, 1H), 3.10 (dd, J = 13.6, 3.4 Hz, 1H), 2.82 (s, 3H), 2.45 (s, 3H), 2.20 (dd, J = 14.1, 7.3 Hz, 1H), 2.01 (d, J = 22.0 Hz, 3H), 1.20 (d, J = 7.0 Hz, 3H), 1.13 (d, J = 6.4 Hz, 3H). |
| 1-22 | 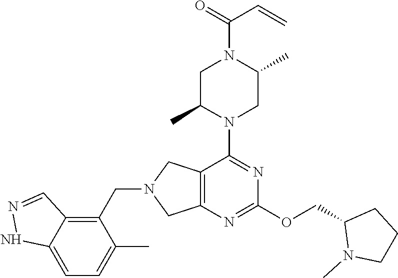 [SMILES: C=CC(=O)N1C[C@H](C)N(c2nc(OC[C@@H]3CCCN3C)nc3c2CN(Cc2c(C)ccc4[nH]ncc24)C3)C[C@H]1C] | A | 545.3 | 1%/ 15% | 1H NMR (400 MHz, CD₃OD) δ 7.14 (d, J = 8.4 Hz, 1H), 6.93-6.65 (m, 3H), 6.35-6.20 (m, 1H), 5.80 (dd, J = 21.3, 10.6 Hz, 1H), 5.14-4.99 (m, 1H), 4.77- 3.93 (m, 8H), 3.68-3.37 (m, 3H), 3.14-2.73 (m, 6H), 2.39-2.17 (m, 4H), 2.14-1.78 (m, 2H), 1.34- 1.01 (m, 3H). |
| 1-23 | 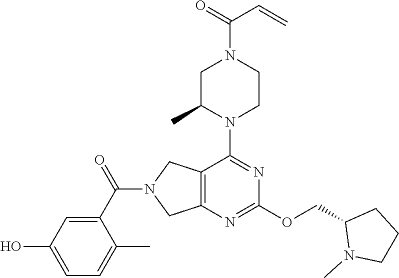 [SMILES: C=CC(=O)N1CCN(c2nc(OC[C@@H]3CCCN3C)nc3c2CN(C(=O)c2cc(O)ccc2C)C3)[C@@H](C)C1] | E | | 9%/ 35% | 1H NMR (400 MHz, CD₃OD) δ 7.91-7.56 (m, 4H), 7.07-6.68 (m, 2H), 6.36- 6.21 (m, 1H), 5.81 (dd, J = 18.8, 10.6 Hz, 1H), 5.17- 4.98 (m, 2H), 4.82-3.89 (m, 8H), 3.70-3.38 (m, 2H), 3.22-2.64 (m, 7H), 2.45-1.79 (m, 4H), 1.35- 1.11 (m, 3H). |
| 1-24 | 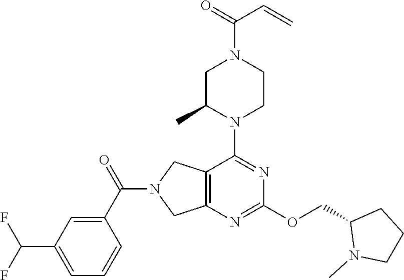 [SMILES: C=CC(=O)N1CCN(c2nc(OC[C@@H]3CCCN3C)nc3c2CN(C(=O)c2cccc(C(F)F)c2)C3)[C@@H](C)C1] | E | | 0%/ 0% | 1H NMR (400 MHz, CDCl₃) δ 8.42 (s, 0H), 7.31 (dd, J = 8.8, 5.1 Hz, 1H), 7.23 (dd, J = 9.2, 3.0 Hz, 1H), 6.91 (td, J = 8.3, 3.1 Hz, 1H), 6.54 (s, 1H), 6.34 (d, J = 16.6 Hz, 1H), 5.78 (d, J = 10.5 Hz, 1H), 4.95 (s, 1H), 4.73 (dd, J = 11.7, 7.1 Hz, 1H), 4.46 (dd, J = 11.8, 4.4 Hz, 2H), 4.21- 4.02 (m, 3H), 4.01-3.69 (m, 5H), 3.57 (dd, J = 16.9, 10.2 Hz, 1H), 3.26 (d, J = 39.9 Hz, 4H), 3.07 (s, 1H), 2.88-2.69 (m, 4H), 2.64 (dd, J = 16.1, 4.4 Hz, 1H), 2.28-1.84 (m, 4H). |
| 1-25 | 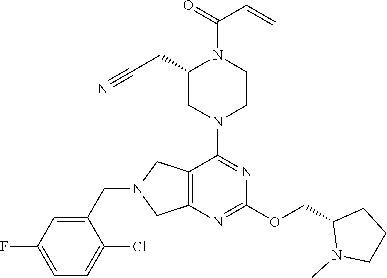 [SMILES: C=CC(=O)N1CCN(c2nc(OC[C@@H]3CCCN3C)nc3c2CN(Cc2cc(F)ccc2Cl)C3)C[C@@H]1CC#N] | A | 554.2 | 78%/ 93% | 1H NMR (400 MHz, CD₃OD) δ 7.30 (d, 1H), 7.18 (s, 1H), 6.72-6.68 (m, 2H), 6.25 (d, 1H), 5.83 (d, 1H), 4.87 (d, 1H), 4.64 (d, 2H), 4.18 (m, 4H), 3.89 (s, 2H), 3.79 (s, 3H), 3.56- 3.55 (m, 1H), 3.12-3.09 (m, 2H), 2.90-2.80 (m, 4H), 2.50 (s, 3H), 2.47-2.43 (m, 1H), 2.36-2.33 (m, 1H), 1.74- 1.73 (m, 2H), 1.67-1.60 (m, 1H). |
| 1-26 | 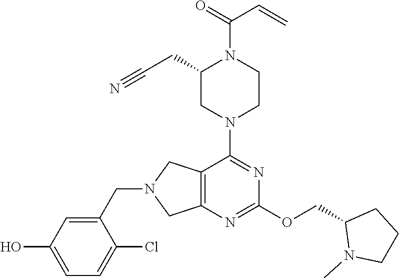 [SMILES: C=CC(=O)N1CCN(c2nc(OC[C@@H]3CCCN3C)nc3c2CN(Cc2cc(O)ccc2Cl)C3)C[C@@H]1CC#N] | D | 552.2 | 74%/ 74% | 1H NMR (400 MHz, CD₃OD) δ 7.27 (ddd, J = 8.7, 5.5, 3.2 Hz, 1H), 7.17-7.01 (m, 2H), 6.80 (d, J = 11.3 Hz, 1H), 6.28 (d, J = 16.6 Hz, 1H), 5.82 (d, J = 10.0 Hz, 1H), 4.59 (t, J = 23.8 Hz, 1H), 4.49-4.31 (m, 2H), 4.17 (s, 3H), 4.07 (d, J = 13.8 Hz, 1H), 3.98 (s, 2H), 3.78 (s, 2H), 3.55 (s, 1H), 3.41 (s, 1H), 3.20- 3.08 (m, 2H), 2.90 (dd, J = 16.1, 8.6 Hz, 3H), 2.57 (s, 3H), 2.50-2.43 (m, 1H), 2.15-2.06 (m, 1H), 1.85 (ddd, J = 21.8, 16.4, 9.0 Hz, 3H), 1.77-1.69 (m, 1H). |
| 1-27 | 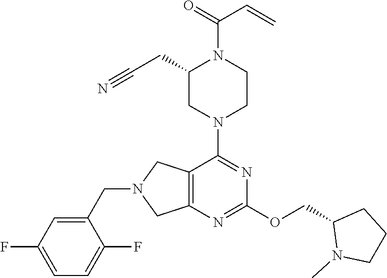 [SMILES: C=CC(=O)N1CCN(c2nc(OC[C@@H]3CCCN3C)nc3c2CN(Cc2cc(F)ccc2F)C3)C[C@@H]1CC#N] | B | 538.3 | 68%/ 97% | 1H NMR (400 MHz, CD₃OD) δ 7.59 (d, J = 2.5 Hz, 1H), 7.40 (d, J = 8.5 Hz, 1H), 7.29 (dd, J = 8.5, 2.6 Hz, 1H), 6.76 (s, 1H), 6.27 (d, J = 16.9 Hz, 1H), 5.81 (d, J = 10.0 Hz, 1H), 4.98 (s, 1H), 4.75 (s, 1H), 4.62 (d, J = 14.1 Hz, 2H), 4.34 (d, J = 6.0 Hz, 2H), 4.20 (s, 3H), 4.04 (s, 3H), 3.80 (s, 2H), 3.54 (s, 1H), 3.14- 3.08 (m, 1H), 2.92-2.73 (m, 3H), 2.53 (s, 3H), 2.44- 2.33 (m, 1H), 2.14-2.04 (m, 1H), 1.84 (dd, J = 14.0, 6.0 Hz, 2H), 1.74-1.62 (m, 1H). |
| 1-28 | 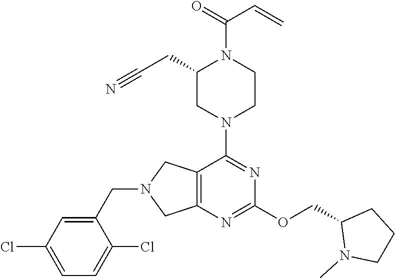 [SMILES: C=CC(=O)N1CCN(c2nc(OC[C@@H]3CCCN3C)nc3c2CN(Cc2cc(Cl)ccc2Cl)C3)C[C@@H]1CC#N] | A | 570.31 | 94%/ 98% | 1H NMR (400 MHz, CD₃OD) δ 7.01 (t, J = 7.5 Hz, 3H), 6.77 (s, 1H), 6.27 (d, J = 16.0 Hz, 1H), 5.81 (d, J = 9.6 Hz, 1H), 4.98 (s, 1H), 4.75 (s, 1H), 4.61 (d, J = 14.4 Hz, 1H), 4.33 (s, 2H), 4.11 (s, 4H), 3.96 (s, 2H), 3.67 (s, 2H), 3.48 (s, 1H), 3.10 (s, 2H), 3.02- 2.65 (m, 4H), 2.52 (d, J = 5.8 Hz, 3H), 2.36 (d, J = 47.6 Hz, 6H), 2.08 (d, J = 7.7 Hz, 1H), 1.82 (s, 2H), 1.70 (s, 1H). |
| 1-29 | 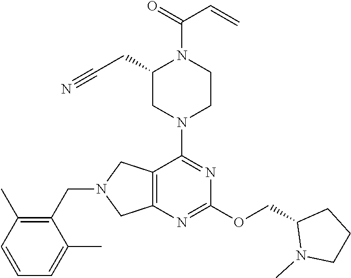 [SMILES: C=CC(=O)N1CCN(c2nc(OC[C@@H]3CCCN3C)nc3c2CN(Cc2c(C)cccc2C)C3)C[C@@H]1CC#N] | A | 530.3 | 98%/ 99% | 1H NMR (400 MHz, CD₃OD) δ 7.37 (s, 1H), 7.17 (d, J = 2.0 Hz, 2H), 6.90-6.69 (m, 1H), 6.27 (d, J = 16.3 Hz, 1H), 5.79 (dd, J = 10.6, 1.8 Hz, 1H), 4.62 (s, 1H), 4.52-4.25 (m, 3H), 4.23-3.96 (m, 4H), 3.89 (s, 2H), 3.70 (s, 2H), 3.54 (d, J = 10.9 Hz, 1H), 3.36 (d, J = 9.4 Hz, 1H), 3.19-2.95 (m, 2H), 2.78 (d, J = 6.7 Hz, 1H), 2.51 (s, 3H), 2.43-2.27 (m, 4H), 2.08 (ddd, J = 16.4, 12.5, 8.3 Hz, 1H), 1.82 (td, J = 13.4, 8.3 Hz, 2H), 1.74-1.63 (m, 1H), 1.20 (d, J = 6.5 Hz, 3H). |
| 1-30 | 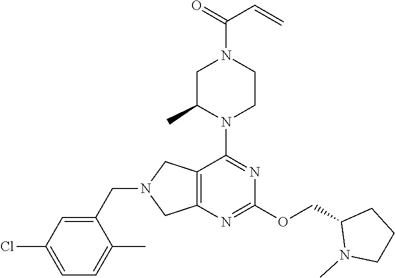 [SMILES: C=CC(=O)N1CCN(c2nc(OC[C@@H]3CCCN3C)nc3c2CN(Cc2cc(Cl)ccc2C)C3)[C@@H](C)C1] | A | 525.3 | 0%/ 7% | 1H NMR (400 MHz, CD₃OD) δ 7.69 (d, J = 8.1 Hz, 2H), 7.58 (d, J = 7.4 Hz, 1H), 7.44 (t, J = 7.7 Hz, 1H), 7.35-7.30 (m, 1H), 7.26 (d, J = 6.9 Hz, 1H), 6.84-6.71 (m, 1H), 6.26 (d, J = 16.5 Hz, 1H), 5.78 (d, J = 10.7 Hz, 1H), 4.71 (d, J = 10.4 Hz, 1H), 4.64 (d, J = 11.3 Hz, 1H), 4.58- 4.45 (m, 2H), 4.36 (dt, J = 24.0, 8.6 Hz, 5H), 4.13 (d, J = 11.1 Hz, 1H), 4.05- 3.89 (m, 1H), 3.54 (d, J = 13.7 Hz, 1H), 3.36 (s, 1H), 3.12 (dd, J = 9.6, 4.6 Hz, 1H), 2.90 (s, 3H), 2.82 (dd, J = 13.5, 6.9 Hz, 1H), 2.54 (s, 3H), 2.40 (dd, J = 17.9, 9.0 Hz, 1H), 2.15-2.07 (m, 1H), 1.84 (td, J = 12.7, 7.9 Hz, 2H), 1.77-1.69 (m, 1H), 1.22 (dd, J = 20.7, 6.5 Hz, 3H). |
| 1-31 | 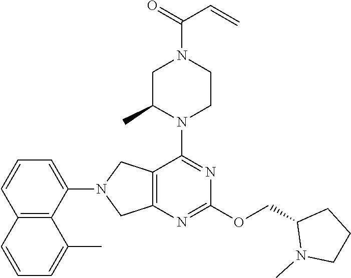 [SMILES: C=CC(=O)N1CCN(c2nc(OC[C@@H]3CCCN3C)nc3c2CN(c2cccc4cccc(C)c24)C3)[C@@H](C)C1] | C | 527.3 | 0%/ 6% | 1H NMR (400 MHz, CD₃OD) δ 6.96-6.74 (m, 3H), 6.72-6.67 (m, 1H), 6.29- 6.21 (m, 1H), 5.82-5.75 (m, 1H), 4.64 (s, 1H), 4.44 (s, 1H), 4.33-4.27 (m, 2H), 4.09 (t, J = 10.1 Hz, 2H), 3.90 (s, 2H), 3.84 (d, J = 14.9 Hz, 1H), 3.74 (s, 2H), 3.65 (dd, J = 14.0, 3.6 Hz, 1H), 3.46 (t, J = 12.4 Hz, 1H), 3.29-3.22 (m, 1H), 3.10 (dt, J = 9.5, 4.6 Hz, 1H), 2.77 (dd, J = 13.4, 6.5 Hz, 1H), 2.51 (s, 3H), 2.38 (dd, J = 17.9, 9.0 Hz, 1H), 2.13-2.04 (m, 1H), 1.86-1.78 (m, 2H), 1.69 (dt, J = 19.7, 7.4 Hz, 1H), 1.30-1.21 (m, 6H). |
| 1-32 | 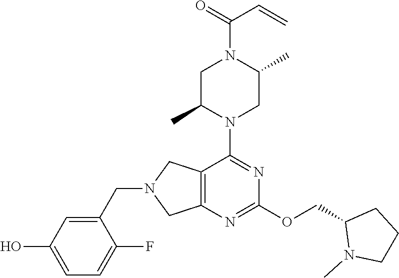 [SMILES: C=CC(=O)N1C[C@H](C)N(c2nc(OC[C@@H]3CCCN3C)nc3c2CN(Cc2cc(O)ccc2F)C3)C[C@H]1C] | A | 525.3 | 7%/ 41% | 1H NMR (400 MHz, CDCl₃) δ 8.36 (s, 1H), 7.43 (dd, J = 6.2, 2.6 Hz, 1H), 7.22 (ddd, J = 8.7, 4.4, 2.8 Hz, 1H), 7.01 (t, J = 9.0 Hz, 1H), 6.57 (s, 1H), 6.35 (dd, J = 16.8, 1.8 Hz, 1H), 5.75 (d, J = 10.3 Hz, 1H), 4.68 (d, J = 28.3 Hz, 2H), 4.44 (dd, J = 11.8, 3.9 Hz, 1H), 4.32 (s, 1H), 4.08 (d, J = 11.3 Hz, 2H), 4.00 (d, J = 20.8 Hz, 1H), 3.92 (s, 2H), 3.82 (s, 2H), 3.67 (s, 1H), 3.43 (s, 1H), 3.28 (s, 1H), 3.18-3.00 (m, 1H), 2.85 (s, 5H), 2.31-2.19 (m, 1H), 2.14 (d, J = 11.3 Hz, 1H), 2.02 (dd, J = 15.7, 7.4 Hz, 2H), 1.21 (t, J = 12.7 Hz, 3H). |
| 1-33 | 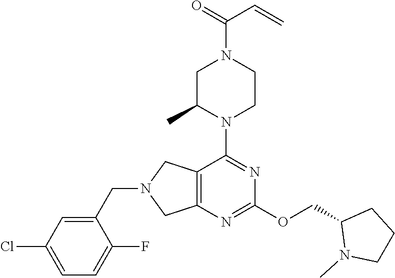 [SMILES: C=CC(=O)N1CCN(c2nc(OC[C@@H]3CCCN3C)nc3c2CN(Cc2cc(Cl)ccc2F)C3)[C@@H](C)C1] | A | 529.2 | 0%/ 5% | 1H NMR (400 MHz, CD₃OD) δ 8.49 (s, 1H), 7.43 (dd, J = 8.8, 5.1 Hz, 1H), 7.35 (dd, J = 9.4, 3.0 Hz, 1H), 7.06 (td, J = 8.4, 3.1 Hz, 1H), 6.80 (td, J = 17.4, 10.7 Hz, 1H), 6.27 (dd, J = 16.7, 2.6 Hz, 1H), 5.80 (dd, J = 10.6, 1.7 Hz, 1H), 4.69 (dd, J = 12.5, 3.1 Hz, 1H), 4.67-4.55 (m, 1H), 4.55- 4.49 (m, 1H), 4.49- 4.33 (m, 1H), 4.20 (q, J = 11.5 Hz, 3H), 4.05 (s, 2H), 4.00 (d, J = 13.6 Hz, 1H), 3.88-3.80 (m, 3H), 3.72- 3.65 (m, 1H), 3.56 (dd, J = 13.6, 2.3 Hz, 1H), 3.40- 3.34 (m, 1H), 3.21 (ddd, J = 25.9, 14.2, 5.4 Hz, 2H), 3.04 (s, 3H), 2.38 (dt, J = 14.6, 8.1 Hz, 1H), 2.22- 2.06 (m, 2H), 2.04-1.94 (m, 1H), 1.22 (d, J = 6.6 Hz, 3H). |
| 1-34 | 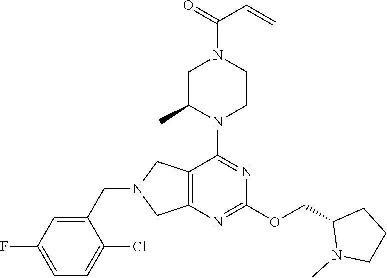 [SMILES: C=CC(=O)N1CCN(c2nc(OC[C@@H]3CCCN3C)nc3c2CN(Cc2cc(F)ccc2Cl)C3)[C@@H](C)C1] | A | 529.2 | 0%/ 14% | 1H NMR (400 MHz, CD₃OD) δ 7.36 (dd, J = 13.9, 7.8 Hz, 1H), 7.19 (dd, J = 21.0, 8.7 Hz, 2H), 7.02 (dd, J = 11.7, 5.3 Hz, 1H), 6.78 (d, J = 12.7 Hz, 1H), 6.27 (d, J = 16.8 Hz, 1H), 5.81 (d, J = 10.1 Hz, 1H), 4.97 (s, 1H), 4.61 (d, J = 13.8 Hz, 1H), 4.44-4.31 (m, 2H), 4.08 (d, J = 33.3 Hz, 4H), 3.93 (s, 2H), 3.73 (s, 2H), 3.53 (s, 1H), 3.37 (s, 1H), 3.22-3.05 (m, 2H), 2.98-2.76 (m, 3H), 2.55 (d, J = 21.8 Hz, 3H), 2.52-2.43 (m, 1H), 2.12 (ddd, J = 16.4, 12.5, 8.1 Hz, 1H), 1.90-1.82 (m, 2H), 1.73 (dt, J = 20.1, 7.3 Hz, 1H). |
| 1-35 | 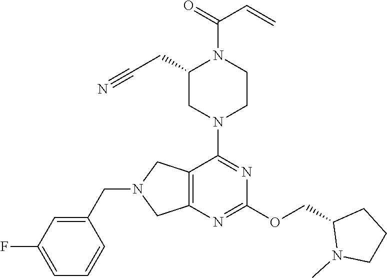 [SMILES: C=CC(=O)N1CCN(c2nc(OC[C@@H]3CCCN3C)nc3c2CN(Cc2cccc(F)c2)C3)C[C@@H]1CC#N] | A | 520.3 | 56%/ 91% | 1H NMR (400 MHz, CD₃OD) δ 7.31 (dd, J = 15.2, 8.3 Hz, 1H), 6.91-6.67 (m, 3H), 6.27 (d, J = 16.5 Hz, 1H), 5.82 (d, J = 10.1 Hz, 1H), 4.62 (d, J = 14.0 Hz, 1H), 4.41-4.29 (m, 2H), 4.16 (s, 2H), 4.09 (d, J = 21.1 Hz, 1H), 4.00 (d, J = 1.7 Hz, 2H), 3.88 (s, 3H), 3.75 (s, 2H), 3.54 (s, 1H), 3.35 (s, 1H), 3.22- 3.06 (m, 2H), 2.88 (dd, J = 16.9, 7.6 Hz, 3H), 2.57 (s, 3H), 2.48 (dd, J = 18.0, 9.0 Hz, 1H), 2.22-1.99 (m, 2H), 1.90 (s, 1H), 1.85 (s, 2H), 1.72 (dt, J = 20.3, 7.4 Hz, 1H). |
| 1-36 | 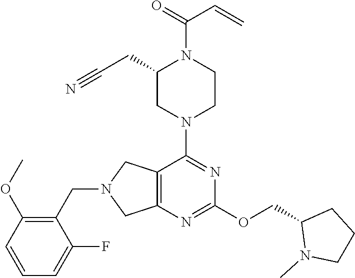 [SMILES: C=CC(=O)N1CCN(c2nc(OC[C@@H]3CCCN3C)nc3c2CN(Cc2c(F)cccc2OC)C3)C[C@@H]1CC#N] | A | 550.2 | 91%/ 97% | 1H NMR (400 MHz, CD₃OD) δ 7.02 (t, J = 14.4 Hz, 1H), 6.84 (d, J = 2.5 Hz, 1H), 6.82-6.72 (m, 1H), 6.64 (dd, J = 8.2, 2.6 Hz, 1H), 6.24 (d, J = 16.6 Hz, 1H), 5.78 (dd, J = 10.7, 1.4 Hz, 1H), 4.87-4.76 (m, 1H), 4.45 (s, 1H), 4.12-4.04 (m, 2H), 4.05 (s, 3H), 3.96-3.85 (m, 4H), 3.80 (d, J = 12.7 Hz, 1H), 3.56 (d, J = 9.3 Hz, 1H), 2.97 (dd, J = 52.7, 23.5 Hz, 2H), 2.28 (s, 3H), 1.31 (s, 3H). |
| 1-37 | 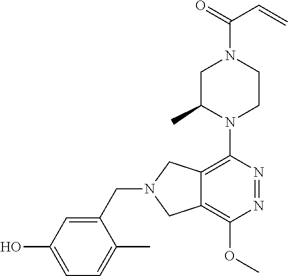 [SMILES: C=CC(=O)N1CCN(c2nnc(OC)c3c2CN(Cc2cc(O)ccc2C)C3)[C@@H](C)C1] | G | 424.2 | 0%/ 0% | 1H NMR (400 MHz, CD₃OD) δ 7.28 (t, J = 8.2 Hz, 1H), 7.01 (dd, J = 19.9, 8.1 Hz, 2H), 6.79 (s, 1H), 6.28 (d, J = 16.2 Hz, 1H), 5.82 (d, J = 10.5 Hz, 1H), 4.99 (s, 1H), 4.76 (s, 1H), 4.61 (d, J = 14.3 Hz, 1H), 4.32 (d, J = 5.8 Hz, 2H), 4.14 (dd, J = 41.1, 18.6 Hz, 5H), 3.88 (s, 3H), 3.80 (s, 2H), 3.54 (s, 1H), 3.39 (d, J = 12.4 Hz, 1H), 3.09 (s, 2H), 2.93-2.73 (m, 3H), 2.51 (s, 3H), 2.38 (d, J = 8.7 Hz, 1H), 2.13-2.02 (m, 1H), 1.87-1.66 (m, 3H). |
| 1-38 | 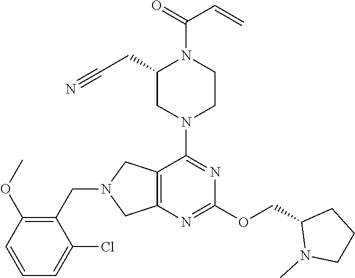 [SMILES: C=CC(=O)N1CCN(c2nc(OC[C@@H]3CCCN3C)nc3c2CN(Cc2c(Cl)cccc2OC)C3)C[C@@H]1CC#N] | A | 566.3 | 97%/ 97% | 1H NMR (400 MHz, CD₃OD) δ 8.50 (s, 1H), 7.13- 6.97 (m, 2H), 6.96-6.66 (m, 2H), 6.28 (d, J = 16.7 Hz, 1H), 5.82 (d, J = 10.3 Hz, 1H), 4.97 (s, 1H), 4.71 (d, J = 8.8 Hz, 2H), 4.46 (dd, J = 12.4, 7.3 Hz, 2H), 4.18 (s, 2H), 4.08 (s, 1H), 3.96 (s, 2H), 3.79 (d, J = 3.4 Hz, 5H), 3.66 (s, 1H), 3.62-3.55 (m, 1H), 3.37 (s, 2H), 3.08 (d, J = 10.8 Hz, 2H), 2.94 (s, 4H), 2.80 (s, 1H), 2.41-2.20 (m, 1H), 2.20-1.75 (m, 3H). |
| 1-39 | 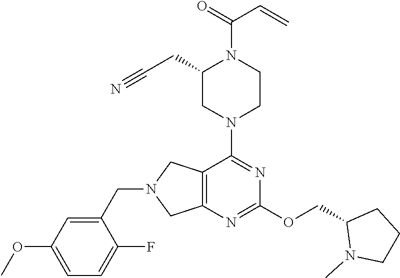 [SMILES: C=CC(=O)N1CCN(c2nc(OC[C@@H]3CCCN3C)nc3c2CN(Cc2cc(OC)ccc2F)C3)C[C@@H]1CC#N] | A | 550.3 | 51%/ 72% | 1H NMR (400 MHz, CD₃OD) δ 6.98-6.96 (m, 1H), 6.83-6.81 (m, 2H), 6.60-6.59 (m, 1H), 6.25 (d, 1H), 5.82 (d, 1H), 4.60 (m, 1H), 4.33-4.32 (m, 2H), 4.11-4.08 (m, 4H), 3.52 (s, 2H), 3.31 (s, 2H), 3.30- 3.28 (m, 1H), 3.13-3.08 (m, 2H), 2.83-2.82 (m, 3H), 2.55 (s, 2H), 2.27-2.26 (m, 1H), 2.10 (s, 3H), 2.07- 2.03 (m, 1H), 1.71-1.68 (m, 3H). |
| 1-40 | 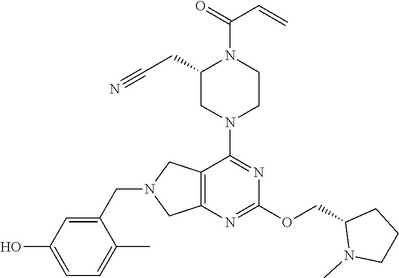 [SMILES: C=CC(=O)N1CCN(c2nc(OC[C@@H]3CCCN3C)nc3c2CN(Cc2cc(O)ccc2C)C3)C[C@@H]1CC#N] | A | 532.2 | 98%/ 98% | 1H NMR (400 MHz, CDCl₃) δ 7.54 (d, J = 2.4 Hz, 1H), 7.32 (d, J = 8.5 Hz, 1H), 7.21 (dd, J = 8.5, 2.5 Hz, 1H), 6.67-6.50 (m, 1H), 6.37 (dd, J = 16.8, 1.8 Hz, 1H), 5.77 (d, J = 10.7 Hz, 1H), 4.66 (s, 1H), 4.42 (s, 2H), 4.23 (s, 1H), 4.11 (q, J = 11.0 Hz, 2H), 4.00 (s, 3H), 3.87 (s, 3H), 3.46 (d, J = 12.0 Hz, 1H), 3.29 (s, 2H), 3.11 (s, 1H), 2.87 (s, 1H), 2.59 (s, 3H), 2.39 (s, 1H), 2.12 (s, 1H), 1.83 (s, 2H), 1.68-1.59 (m, 1H), 1.21 (d, J = 6.7 Hz, 3H). |
| 1-41 | 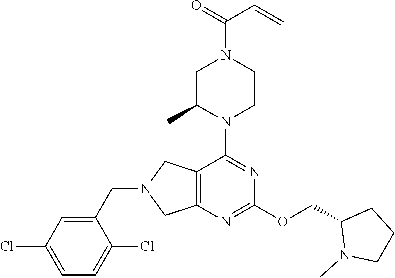 [SMILES: C=CC(=O)N1CCN(c2nc(OC[C@@H]3CCCN3C)nc3c2CN(Cc2cc(Cl)ccc2Cl)C3)[C@@H](C)C1] | A | 545.2 | 3%/ 22% | 1H NMR (400 MHz, CD₃OD) δ 7.42 (d, J = 7.1 Hz, 1H), 7.28-7.16 (m, 3H), 6.76 (s, 1H), 6.32-6.21 (m, 1H), 5.81 (d, J = 10.4 Hz, 1H), 4.93 (d, J = 19.5 Hz, 1H), 4.75 (d, J = 11.5 Hz, 2H), 4.50 (ddd, J = 19.6, 10.0, 5.9 Hz, 2H), 4.25 (q, J = 11.8 Hz, 2H), 4.07 (d, J = 20.6 Hz, 1H), 3.88 (d, J = 16.4 Hz, 2H), 3.76 (d, J = 5.2 Hz, 1H), 3.67-3.58 (m, 1H), 3.45 (d, J = 60.1 Hz, 3H), 3.24-3.04 (m, 3H), 2.98 (s, 3H), 2.94-2.70 (m, 3H), 2.37-2.25 (m, 2H), 2.24-2.10 (m, 2H), 2.09-1.93 (m, 2H). |
| 1-42 | 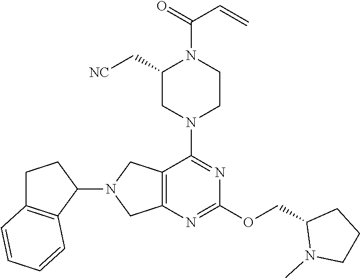 [SMILES: C=CC(=O)N1CCN(c2nc(OC[C@@H]3CCCN3C)nc3c2CN(C2CCc4ccccc42)C3)C[C@@H]1CC#N] | A | 528.2 | 84%/ 95% | 1H NMR (400 MHz, CDCl₃) δ 7.16 (d, J = 8.6 Hz, 1H), 6.97 (d, J = 2.7 Hz, 1H), 6.71 (dd, J = 8.6, 2.9 Hz, 1H), 6.53 (d, J = 15.9 Hz, 1H), 6.35 (dd, J = 16.8, 1.8 Hz, 1H), 5.75 (d, J = 10.1 Hz, 1H), 4.60 (s, 1H), 4.47 (s, 1H), 4.38 (s, 1H), 4.24 (s, 1H), 4.06 (dd, J = 29.0, 11.0 Hz, 2H), 3.91 (t, J = 12.8 Hz, 2H), 3.85- 3.69 (m, 3H), 3.42 (d, J = 12.6 Hz, 1H), 3.24 (s, 3H), 2.87 (s, 2H), 2.59 (s, 3H), 2.45 (s, 1H), 2.09 (s, 1H), 1.92 (s, 1H), 1.81 (s, 2H), 1.16 (d, J = 6.7 Hz, 3H). |
| 1-43 | 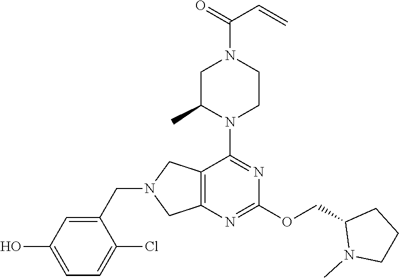 [SMILES: C=CC(=O)N1CCN(c2nc(OC[C@@H]3CCCN3C)nc3c2CN(Cc2cc(O)ccc2Cl)C3)[C@@H](C)C1] | A | 527.2 | 97%/ 98% | 1H NMR (400 MHz, CD₃OD) δ 7.43 (d, J = 8.0 Hz, 2H), 7.29 (dd, J = 8.6, 7.5 Hz, 1H), 6.77 (s, 1H), 6.28 (d, J = 16.7 Hz, 1H), 5.82 (d, J = 10.3 Hz, 1H), 4.97 (s, 1H), 4.72 (m, 2H), 4.43 (d, J = 12.3 Hz, 1H), 4.27 (d, J = 18.6 Hz, 4H), 4.10 (s, 1H), 3.87 (s, 2H), 3.51 (m, J = 23.4 Hz, 3H), 3.14 (m, J = 50.2, 22.7 Hz, 3H), 2.91 (s, 4H), 2.79 (s, 1H), 2.38-2.22 (m, 1H), 2.15-1.86 (m, 4H). |
| 1-44 | 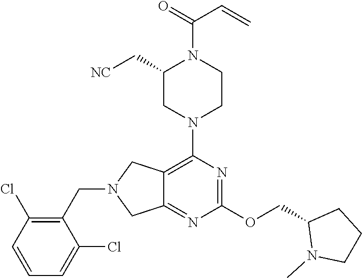 [SMILES: C=CC(=O)N1CCN(c2nc(OC[C@@H]3CCCN3C)nc3c2CN(Cc2c(Cl)cccc2Cl)C3)C[C@@H]1CC#N] | A | 570.3 | 98%/ 100% | 1H NMR (400 MHz, CD₃OD) δ 7.33 (d, J = 5.4 Hz, 1H), 7.18-7.13 (m, 3H), 6.78 (d, J = 12.1 Hz, 1H), 6.27 (d, J = 16.7 Hz, 1H), 5.81 (d, J = 10.0 Hz, 1H), 4.99 (s, 1H), 4.77-4.47 (m, 2H), 4.32 (d, J = 5.6 Hz, 2H), 4.17 (s, 1H), 4.11 (s, 2H), 4.06 (d, J = 15.8 Hz, 1H), 3.91 (s, 2H), 3.70 (s, 2H), 3.55-3.36 (m, 1H), 3.09 (ddd, J = 14.3, 6.4, 3.0 Hz, 2H), 2.91-2.71 (m, 3H), 2.49 (s, 3H), 2.39-2.32 (m, 4H), 2.11-2.02 (m, 1H), 1.84-1.77 (m, 2H), 1.72-1.63 (m, 1H). |
| 1-45 | 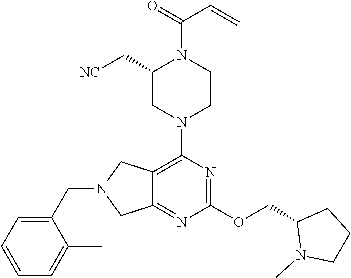 [SMILES: C=CC(=O)N1CCN(c2nc(OC[C@@H]3CCCN3C)nc3c2CN(Cc2ccccc2C)C3)C[C@@H]1CC#N] | A | 516.3 | 94%/ 98% | 1H NMR (400 MHz, CD₃OD) δ 8.52 (s, 1H), 6.94- 6.68 (m, 3H), 6.27 (d, J = 16.7 Hz, 1H), 5.82 (d, J = 10.1 Hz, 1H), 4.94 (d, J = 16.8 Hz, 1H), 4.74 (d, J = 9.5 Hz, 2H), 4.47 (d, J = 7.1 Hz, 1H), 4.17 (s, 2H), 4.08 (s, 1H), 3.91 (s, 2H), 3.76 (d, J = 20.3 Hz, 3H), 3.60 (s, 2H), 3.36 (s, 2H), 3.12 (s, 2H), 2.97 (s, 4H), 2.74 (d, J = 39.6 Hz, 1H), 2.32 (dt, J = 14.4, 7.2 Hz, 1H), 2.23-1.82 (m, 3H). |
| 1-46 | 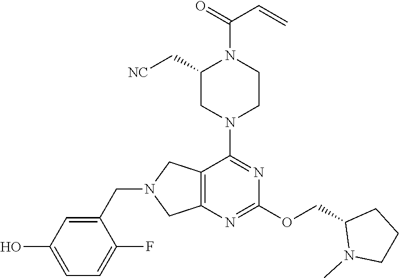 [SMILES: C=CC(=O)N1CCN(c2nc(OC[C@@H]3CCCN3C)nc3c2CN(Cc2cc(O)ccc2F)C3)C[C@@H]1CC#N] | A | 536.3 | 98%/ 99% | 1H NMR (400 MHz, CD₃OD) δ 8.40 (s, 1H), 7.15 (td, J = 8.2, 6.8 Hz, 1H), 6.79 (td, J = 17.2, 10.7 Hz, 1H), 6.66-6.55 (m, 2H), 6.28 (d, J = 16.6 Hz, 1H), 5.80 (dd, J = 10.6, 1.8 Hz, 1H), 4.67 (dd, J = 12.5, 3.2 Hz, 1H), 4.60-4.45 (m, 2H), 4.38 (d, J = 13.5 Hz, 1H), 4.26-4.14 (m, 3H), 4.10 (s, 2H), 3.99 (d, J = 13.8 Hz, 1H), 3.84 (d, J = 7.7 Hz, 2H), 3.83-3.75 (m, 1H), 3.71-3.62 (m, 1H), 3.55 (d, J = 10.9 Hz, 1H), 3.38 (s, 1H), 3.26-3.04 (m, 2H), 3.01 (s, 3H), 2.40-2.30 (m, 1H), 2.21-2.05 (m, 2H), 2.03-1.95 (m, 1H), 1.21 (d, J = 6.5 Hz, 3H). |
| 1-47 | 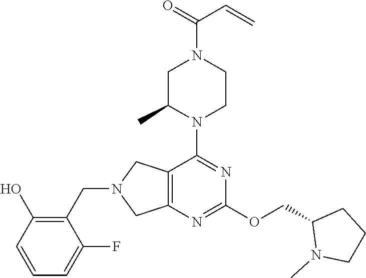 [SMILES: C=CC(=O)N1CCN(c2nc(OC[C@@H]3CCCN3C)nc3c2CN(Cc2c(O)cccc2F)C3)[C@@H](C)C1] | A | 511.3 | 11%/ 43% | 1H NMR (400 MHz, CD₃OD) δ 7.69 (d, J = 8.1 Hz, 2H), 7.59 (d, J = 6.9 Hz, 1H), 7.44 (t, J = 7.7 Hz, 1H), 7.35-7.30 (m, 1H), 7.27 (d, J = 6.8 Hz, 1H), 6.73 (d, J = 10.1 Hz, 1H), 6.21 (d, J = 16.7 Hz, 1H), 5.77 (d, J = 9.4 Hz, 1H), 5.18 (s, 1H), 4.62 (d, J = 60.7 Hz, 3H), 4.51-4.24 (m, 5H), 4.01 (s, 1H), 3.48 (s, 1H), 3.27-3.02 (m, 3H), 2.91 (d, J = 9.0 Hz, 3H), 2.81 (s, 1H), 2.54 (s, 5H), 2.41 (dd, J = 18.1, 9.1 Hz, 1H), 2.17-2.06 (m, 1H), 1.85 (td, J = 12.6, 7.7 Hz, 2H), 1.77-1.67 (m, 1H). |
| 1-48 | 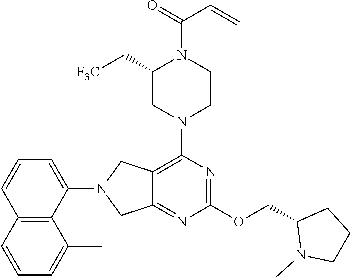 [SMILES: C=CC(=O)N1CCN(c2nc(OC[C@@H]3CCCN3C)nc3c2CN(c2cccc4cccc(C)c24)C3)C[C@@H]1CC(F)(F)F] | G | 595.3 | 0%/ 1% | 1H NMR (400 MHz, CD₃OD) δ 8.47 (s, 1H), 6.92 (t, J = 9.2 Hz, 1H), 6.88 (dd, J = 5.9, 3.0 Hz, 1H), 6.85- 6.73 (m, 1H), 6.73-6.68 (m, 1H), 6.28 (d, J = 16.6 Hz, 1H), 5.80 (dd, J = 10.6, 1.7 Hz, 1H), 4.67 (dd, J = 12.5, 2.9 Hz, 1H), 4.50 (dd, J = 12.4, 7.3 Hz, 2H), 4.38 (d, J = 13.2 Hz, 1H), 4.21-4.11 (m, 3H), 4.00 (d, J = 13.9 Hz, 1H), 3.93 (d, J = 13.9 Hz, 2H), 3.84- 3.74 (m, 3H), 3.72-3.65 (m, 1H), 3.55 (d, J = 11.8 Hz, 1H), 3.40-3.34 (m, 1H), 3.19 (dt, J = 15.5, 7.9 Hz, 2H), 3.02 (s, 3H), 2.35 (dt, J = 14.5, 7.1 Hz, 1H), 2.20-2.05 (m, 2H), 2.03-1.93 (m, 1H), 1.21 (d, J = 6.5 Hz, 3H). |
| 1-49 | 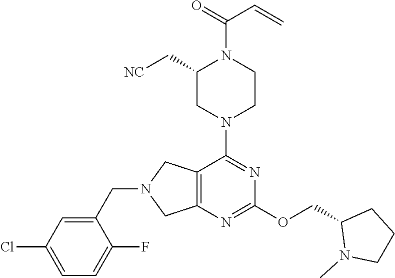 [SMILES: C=CC(=O)N1CCN(c2nc(OC[C@@H]3CCCN3C)nc3c2CN(Cc2cc(Cl)ccc2F)C3)C[C@@H]1CC#N] | A | 554.2 | 81%/ 97% | 1H NMR (400 MHz, CD₃OD) δ 7.12 (t, J = 8.1 Hz, 1H), 6.89 (d, J = 8.0 Hz, 1H), 6.74 (d, J = 8.2 Hz, 2H), 6.27 (d, J = 16.4 Hz, 1H), 5.81 (d, J = 9.9 Hz, 1H), 4.99 (s, 1H), 4.79-4.46 (m, 2H), 4.34 (d, J = 6.3 Hz, 2H), 4.24 (d, J = 4.9 Hz, 3H), 4.09 (d, J = 43.4 Hz, 2H), 3.83 (s, 2H), 3.55 (s, 1H), 3.40 (s, 1H), 3.22-3.03 (m, 2H), 2.98-2.76 (m, 3H), 2.52 (s, 3H), 2.40 (dd, J = 17.7, 8.9 Hz, 1H), 2.13-2.02 (m, 1H), 1.88-1.66 (m, 3H). |
| 1-50 | 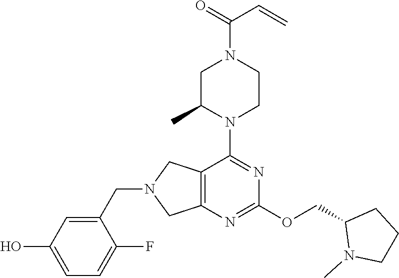 [SMILES: C=CC(=O)N1CCN(c2nc(OC[C@@H]3CCCN3C)nc3c2CN(Cc2cc(O)ccc2F)C3)[C@@H](C)C1] | A | 511.3 | 53%/ 92% | 1H NMR (400 MHz, CD₃OD) δ 8.33 (s, 2H), 7.45 (s, 1H), 7.12 (d, 3H), 6.76-6.73 (m, 1H), 6.29 (d, 1H), 5.83 (s, 1H), 5.11 (s, 1H), 4.78-4.76 (m, 2H), 4.53- 4.62 (m, 1H), 4.32-4.31 (m, 1H), 4.25-4.01 (m, 4H), 3.92-3.84 (m, 2H), 3.64-3.55 (m, 2H), 3.53-3.51 (m, 1H), 3.49-3.28 (m, 1H), 3.27-3.21 (m, 1H), 3.15 (s, 3H), 2.75-2.3 (m, 4H), 2.02-1.77 (m, 9H). |
| 1-51 | 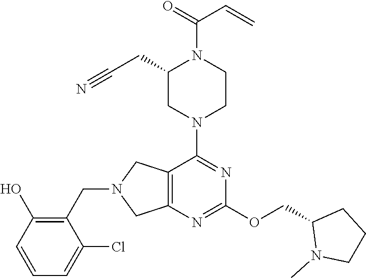 [SMILES: C=CC(=O)N1CCN(c2nc(OC[C@@H]3CCCN3C)nc3c2CN(Cc2c(O)cccc2Cl)C3)C[C@@H]1CC#N] | A | 552.2 | 98%/ 98% | 1H NMR (400 MHz, CD₃OD) δ 8.42 (s, 1H), 7.51- 7.38 (m, 1H), 7.20-7.05 (m, 3H), 6.76 (s, 1H), 6.28 (d, J = 16.5 Hz, 1H), 5.82 (d, J = 10.2 Hz, 1H), 4.94 (d, J = 14.0 Hz, 1H), 4.76 (d, J = 12.4 Hz, 2H), 4.47 (dd, J = 12.5, 7.4 Hz, 1H), 4.29 (d, J = 11.5 Hz, 1H), 4.25-3.96 (m, 4H), 3.90 (d, J = 14.8 Hz, 1H), 3.80 (s, 1H), 3.75-3.62 (m, 2H), 3.48 (d, J = 1.7 Hz, 1H), 3.36 (s, 1H), 3.26-3.08 (m, 2H), 3.00 (s, 3H), 2.95- 2.85 (m, 2H), 2.78 (dt, J = 16.6, 6.1 Hz, 2H), 2.42- 2.29 (m, 1H), 2.20-1.93 (m, 6H), 1.79 (dt, J = 16.6, 10.4 Hz, 1H). |
| 1-52 | 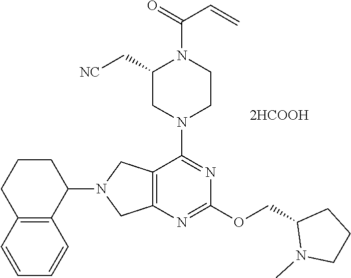 [SMILES: C=CC(=O)N1CCN(c2nc(OC[C@@H]3CCCN3C)nc3c2CN(C2CCCc4ccccc42)C3)C[C@@H]1CC#N] | A | 542.3 | 6%/ 91% | 1H NMR (400 MHz, CD₃OD) δ 7.45 (d, J = 8.7 Hz, 1H), 7.38 (d, J = 2.7 Hz, 1H), 7.13-7.04 (m, 1H), 6.86 (t, J = 73.8 Hz, 2H), 6.28 (d, J = 16.7 Hz, 1H), 5.82 (d, J = 9.9 Hz, 1H), 4.99 (s, 1H), 4.62 (d, J = 14.1 Hz, 2H), 4.36 (d, J = 6.2 Hz, 2H), 4.21 (s, 3H), 4.06 (s, 2H), 3.82 (s, 2H), 3.38 (s, 2H), 3.20-3.05 (m, 2H), 2.95-2.77 (m, 3H), 2.55 (s, 3H), 2.43 (d, J = 9.4 Hz, 1H), 2.15-2.05 (m, 1H), 1.89-1.80 (m, 2H), 1.72 (dd, J = 12.5, 6.9 Hz, 1H). |
| 1-52 (2nd iso- mer) | 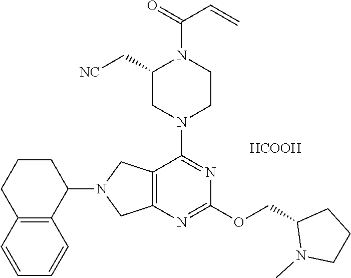 [SMILES: C=CC(=O)N1CCN(c2nc(OC[C@@H]3CCCN3C)nc3c2CN(C2CCCc4ccccc42)C3)C[C@@H]1CC#N ; "HCOOH"] | G | 542.3 | 76%/ 91% | 1H NMR (400 MHz, CD₃OD) δ 8.42 (s, 1H), 7.51- 7.38 (m, 1H), 7.20-7.05 (m, 3H), 6.76 (s, 1H), 6.28 (d, J = 16.5 Hz, 1H), 5.82 (d, J = 10.2 Hz, 1H), 4.94 (d, J = 14.0 Hz, 1H), 4.76 (d, J = 12.4 Hz, 2H), 4.47 (dd, J = 12.5, 7.4 Hz, 1H), 4.29 (d, J = 11.5 Hz, 1H), 4.25-3.96 (m, 4H), 3.90 (d, J = 14.8 Hz, 1H), 3.80 (s, 1H), 3.75-3.62 (m, 2H), 3.48 (d, J = 1.7 Hz, 1H), 3.36 (s, 1H), 3.26-3.08 (m, 2H), 3.00 (s, 3H), 2.95- 2.85 (m, 2H), 2.78 (dt, J = 16.6, 6.1 Hz, 2H), 2.42- 2.29 (m, 1H), 2.20-1.93 (m, 6H), 1.79 (dt, J = 16.6, 10.4 Hz, 1H). |
| 1-53 | 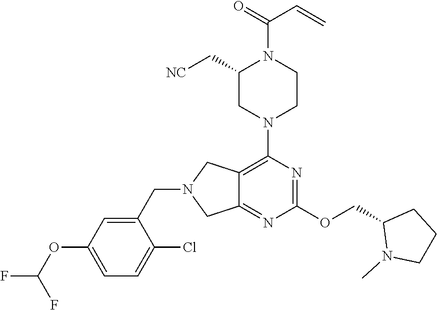 [SMILES: C=CC(=O)N1CCN(c2nc(OC[C@@H]3CCCN3C)nc3c2CN(Cc2cc(OC(F)F)ccc2Cl)C3)C[C@@H]1CC#N] | A | 602.2 | 40%/ 86% | 1H NMR (400 MHz, CDCl₃) δ 7.00 (d, J = 8.4 Hz, 2H), 6.67-6.61 (m, 1H), 6.52 (d, J = 27.1 Hz, 1H), 6.35 (dd, J = 16.8, 1.8 Hz, 1H), 5.74 (d, J = 10.6 Hz, 1H), 4.65-4.46 (m, 2H), 4.38 (s, 1H), 4.29-4.21 (m, 1H), 3.94 (d, J = 11.8 Hz, 1H), 3.87-3.83 (m, 1H), 3.74 (dd, J = 23.9, 14.6 Hz, 3H), 3.43 (s, 1H), 3.23 (s, 2H), 3.05 (s, 1H), 2.94 (s, 1H), 2.82 (s, 1H), 2.60 (s, 3H), 2.48 (s, 1H), 2.27 (d, J = 8.5 Hz, 3H), 2.11 (s, 1H), 2.03 (s, 1H), 1.94 (s, 1H), 1.84 (s, 2H), 1.35 (d, J = 6.4 Hz, 3H), 1.18-1.11 (m, 3H). |
| 1-54 | 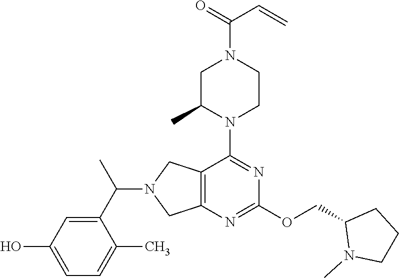 [SMILES: C=CC(=O)N1CCN(c2nc(OC[C@@H]3CCCN3C)nc3c2CN(C(C)c2cc(O)ccc2C)C3)[C@@H](C)C1] | A | 521.3 | 70%/ 77% | 1H NMR (400 MHz, DMSO) δ 8.19 (s, 1H), 6.99 (t, J = 7.8 Hz, 1H), 6.80 (m, 1H), 6.63 (d, J = 7.8 Hz, 2H), 6.17 (d, J = 16.6 Hz, 1H), 5.76 (d, J = 10.0 Hz, 1H), 5.31 (m, 1H), 4.80 (d, J = 64.2 Hz, 2H), 4.25 (m, 3H), 4.08 (m, 3H), 3.95 (s, 2H), 3.68 (s, 2H), 2.94 (m, 4H), 2.56 (d, J = 6.2 Hz, 1H), 2.32 (d, J = 27.9 Hz, 6H), 2.21 (d, J = 8.6 Hz, 1H), 1.95 (d, J = 24.8 Hz, 2H), 1.67 (m, 3H). |
| 1-55 | 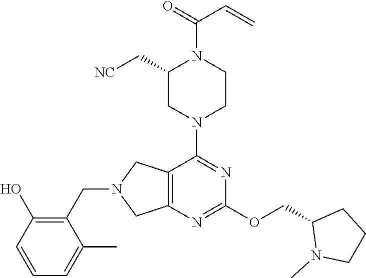 [SMILES: C=CC(=O)N1CCN(c2nc(OC[C@@H]3CCCN3C)nc3c2CN(Cc2c(C)cccc2O)C3)C[C@@H]1CC#N] | A | 532.3 | 78%/ 91% | 1H NMR (400 MHz, CD₃OD) δ 6.99 (d, J = 8.2 Hz, 1H), 6.85-6.75 (m, 2H), 6.63 (dd, J = 8.2, 2.6 Hz, 1H), 6.23 (d, J = 16.7 Hz, 1H), 5.77 (dd, J = 10.7, 1.6 Hz, 1H), 4.64-4.57 (m, 2H), 4.45 (s, 1H), 4.17- 4.01 (m, 3H), 3.90 (d, J = 11.4 Hz, 4H), 3.81 (d, J = 12.6 Hz, 1H), 3.57 (d, J = 11.0 Hz, 1H), 3.26-3.18 (m, 1H), 3.13-3.01 (m, 3H), 2.95 (s, 1H), 2.53 (s, 6H), 2.28 (s, 3H), 1.40-1.36 (m, 3H). |
| 1-56 | 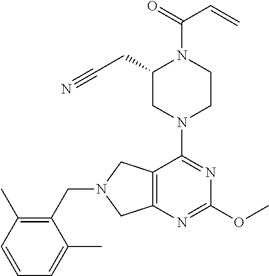 [SMILES: C=CC(=O)N1CCN(c2nc(OC)nc3c2CN(Cc2c(C)cccc2C)C3)C[C@@H]1CC#N] | G | 481.2 | 0%/ 0% | 1H NMR (400 MHz, CD₃OD) δ 7.04 (dt, J = 14.9, 6.0 Hz, 3H), 6.77 (s, 1H), 6.27 (d, J = 17.2 Hz, 1H), 5.81 (d, J = 10.8 Hz, 1H), 5.00 (s, 1H), 4.81-4.38 (m, 2H), 4.19 (s, 1H), 4.10 (s, 2H), 3.94 (d, J = 15.5 Hz, 5H), 3.67 (s, 2H), 3.53 (s, 1H), 3.35 (s, 1H), 3.13 (s, 1H), 2.88 (dd, J = 16.6, 7.5 Hz, 2H), 2.42 (s, 6H). |
| 1-57 | 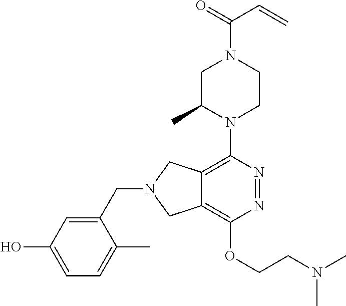 [SMILES: C=CC(=O)N1CCN(c2nnc(OCCN(C)C)c3c2CN(Cc2cc(O)ccc2C)C3)[C@@H](C)C1] | A | 447.2 | 0%/ 0% | 1H NMR (400 MHz, CD₃OD) δ 7.40 (dd, J = 14.9, 8.3 Hz, 1H), 7.14-6.70 (m, 4H), 6.27 (d, J = 16.6 Hz, 1H), 5.82 (d, J = 10.1 Hz, 1H), 4.98 (s, 1H), 4.78- 4.46 (m, 2H), 4.34 (d, J = 6.5 Hz, 2H), 4.18 (s, 3H), 4.03 (d, J = 1.2 Hz, 2H), 3.77 (s, 2H), 3.51 (d, J = 26.8 Hz, 1H), 3.38 (d, J = 16.7 Hz, 1H), 3.15 (d, J = 4.3 Hz, 2H), 2.88 (dd, J = 16.5, 7.5 Hz, 3H), 2.56 (s, 3H), 2.47 (d, J = 8.9 Hz, 1H), 2.11 (dd, J = 12.5, 8.0 Hz, 1H), 1.90-1.65 (m, 3H). |
| 1-58 | 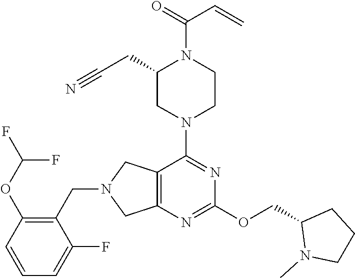 [SMILES: C=CC(=O)N1CCN(c2nc(OC[C@@H]3CCCN3C)nc3c2CN(Cc2c(F)cccc2OC(F)F)C3)C[C@@H]1CC#N] | A | 586.3 | 96%/ 98% | 1H NMR (400 MHz, CD₃OD) δ 8.56 (s, 1H), 7.53 (dd, J = 6.1, 2.5 Hz, 1H), 7.33 (ddd, J = 8.1, 4.1, 2.8 Hz, 1H), 7.13 (t, J = 9.1 Hz, 1H), 6.81 (dt, J = 17.0, 16.3 Hz, 1H), 6.28 (d, J = 16.7 Hz, 1H), 5.82 (d, J = 9.9 Hz, 1H), 4.96 (d, J = 10.1 Hz, 1H), 4.76 (d, J = 9.5 Hz, 2H), 4.51 (dd, J = 12.4, 7.4 Hz, 1H), 4.19 (s, 2H), 4.12-4.04 (m, 1H), 3.98 (s, 2H), 3.80 (s, 3H), 3.71-3.63 (m, 1H), 3.61- 3.47 (m, 1H), 3.45-3.32 (m, 2H), 3.19 (dd, J = 18.6, 8.1 Hz, 2H), 3.01 (s, 3H), 2.95-2.87 (m, 1H), 2.80 (dd, J = 16.3, 15.2 Hz, 1H), 2.42-2.30 (m, 1H), 2.20- 2.05 (m, 2H), 2.01 (dd, J = 13.9, 6.6 Hz, 1H). |
| 1-59 | 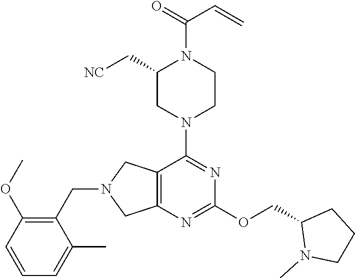 [SMILES: C=CC(=O)N1CCN(c2nc(OC[C@@H]3CCCN3C)nc3c2CN(Cc2c(C)cccc2OC)C3)C[C@@H]1CC#N] | A | 546.3 | 46%/ 98% | 1H NMR (400 MHz, CD₃OD) δ 8.35 (s, 2H), 7.21 (t, J = 8.0 Hz, 1H), 6.86 (dd, J = 14.9, 7.9 Hz, 3H), 6.29 (d, J = 16.7 Hz, 1H), 5.83 (d, J = 10.3 Hz, 1H), 4.95 (d, J = 8.4 Hz, 1H), 4.76 (t, J = 11.2 Hz, 2H), 4.50 (dd, J = 12.6, 7.4 Hz, 1H), 4.31 (s, 2H), 4.09 (d, J = 12.3 Hz, 4H), 3.84 (m, 6H), 3.67 (m, 1H), 3.40 (s, 2H), 3.21 (dt, J = 11.2, 8.0 Hz, 2H), 3.02 (s, 3H), 2.91 (s, 2H), 2.42 (s, 3H), 2.36 (dd, J = 12.8, 6.2 Hz, 1H), 2.08 (m, 3H). |
| 1-61 | 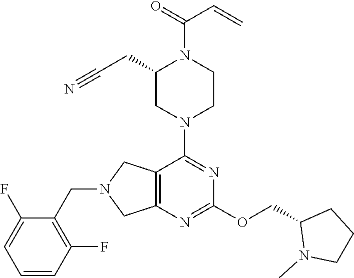 [SMILES: C=CC(=O)N1CCN(c2nc(OC[C@@H]3CCCN3C)nc3c2CN(Cc2c(F)cccc2F)C3)C[C@@H]1CC#N] | A | M + 1 = 538.3 | NA/ 80% | 1H NMR (400 MHz, CD₃OD) δ 7.33-7.31 (m, 1H), 7.29 (m, 2H), 6.9 (br, 1H), 6.20 (d, 1H), 5.74 (d, 1H), 3.63-3.62 (m, 1H), 3.54 (d, 2H), 3.27 (d, 2H), 3.26- 3.25 (m, 4H), 3.21 (s, 2H), 3.01 (s, 2H), 3.03-3.01 (m, 1H), 2.92-2.80 (m, 2H), 2.79-2.76 (m, 3H), 2.37 (s, 3H), 2.30-2.29 (m, 1H), 2.01-2.03 (m, 1H), 1.72- 1.70 (m, 2H), 1.61-1.40 (m, 1H). |
| 1-64 | 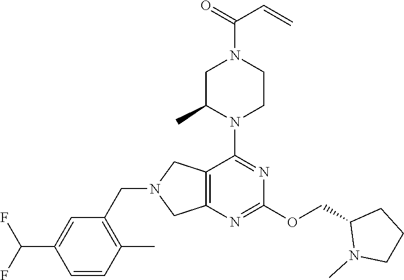 [SMILES: C=CC(=O)N1CCN(c2nc(OC[C@@H]3CCCN3C)nc3c2CN(Cc2cc(C(F)F)ccc2C)C3)[C@@H](C)C1] | A | M + 1 = 541.3 | 0%/ 0% | 1H NMR (400 MHz, CD₃OD) δ 7.54 (s, 1H), 7.45- 7.25 (m, 2H), 6.92-6.56 (m, 2H), 6.28 (dd, J = 17.1, 5.1 Hz, 1H), 5.80 (d, J = 10.6 Hz, 1H), 4.75-4.42 (m, 1H), 4.42-4.31 (m, 2H), 4.25-4.08 (m, 3H), 3.97 (s, 2H), 3.71 (s, 2H), 3.58-3.36 (m. 1H), 3.27- 2.96 (m, 3H), 2.66-2.51 (m, 4H), 2.45 (s, 3H), 2.27- 1.67 (m, 4H), 1.21 (d, J = 6.7 Hz, 3H). |
| 1-65 | 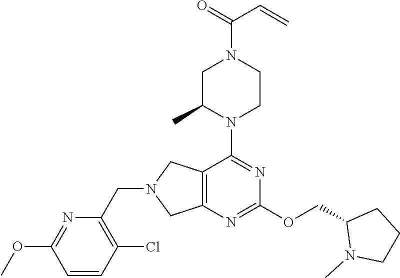 [SMILES: C=CC(=O)N1CCN(c2nc(OC[C@@H]3CCCN3C)nc3c2CN(Cc2nc(OC)ccc2Cl)C3)[C@@H](C)C1] | E | M + 1 = 542.3 | 0%/ 2% | Intermediate. No NMR |
| 1-66 | 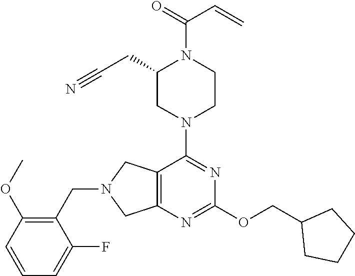 [SMILES: C=CC(=O)N1CCN(c2nc(OCC3CCCC3)nc3c2CN(Cc2c(F)cccc2OC)C3)C[C@@H]1CC#N] | E | M + 1 = 535.3 | 1%/ 14% | 1H NMR (400 MHz, CD₃OD) δ 7.31 (td, J = 8.4, 6.9 Hz, 1H), 6.91-6.72 (m, 3H), 6.27 (d, J = 16.6 Hz, 1H), 5.82 (d, J = 10.3 Hz, 1H), 4.99 (s, 1H), 4.81- 4.45 (m, 2H), 4.22-4.09 (m, 5H), 3.99 (d, J = 1.7 Hz, 2H), 3.88 (s, 3H), 3.74 (s, 2H), 3.52 (t, J = 15.9 Hz, 1H), 3.37 (d, J = 17.5 Hz, 1H), 3.13 (d, J = 1.6 Hz, 1H), 3.00-2.76 (m, 2H), 2.33 (dt, J = 15.0, 7.5 Hz, 1H), 1.81 (td, J = 11.5, 6.6 Hz, 2H), 1.70-1.53 (m, 4H), 1.36 (dt, J = 19.1, 6.7 Hz, 2H). |
| 1-67 | 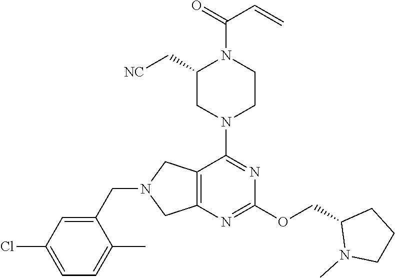 [SMILES: C=CC(=O)N1CCN(c2nc(OC[C@@H]3CCCN3C)nc3c2CN(Cc2cc(Cl)ccc2C)C3)C[C@@H]1CC#N] | E | M + 1 = 550.3 | 70%/ 92% | 1H NMR (400 MHz, CD₃OD) δ 8.52 (s, 0.1H), 7.36 (s, 1H), 7.20-7.12 (m, 2H), 6.75 (s, 1H), 6.26 (d, J = 16.8 Hz, 1H), 5.80 (d, J = 10.4 Hz, 1H), 4.96 (s, 1H), 4.71-4.53 (m, 2H), 4.44- 4.35 (m, 1H), 4.11 (d, J = 12.0 Hz, 4H), 3.89 (s, 2H), 3.73 (s, 2H), 3.57-3.31 (m, 4H), 3.12 (s, 1H), 2.94- 2.69 (m, 6H), 2.35 (s, 3H), 2.21 (dd, J = 17.5, 10.0 Hz, 1H), 2.04-1.93 (m, 2H), 1.89-1.79 (m, 1H). |
| 1-68 | 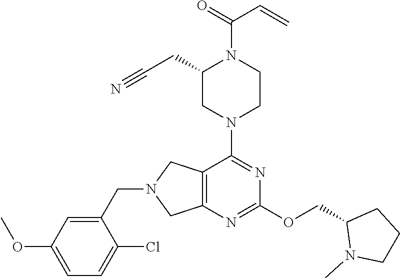 [SMILES: C=CC(=O)N1CCN(c2nc(OC[C@@H]3CCCN3C)nc3c2CN(Cc2cc(OC)ccc2Cl)C3)C[C@@H]1CC#N] | A | M + 1 = 566.3 | 90%/ 97% | 1H NMR (400 MHz, CDCl₃) δ 8.35 (s, 1H), 7.22 (d, J = 2.0 Hz, 1H), 7.01 (d, J = 2.9 Hz, 1H), 6.73 (dd, J = 8.8, 3.0 Hz, 1H), 6.51 (s, 1H), 6.33 (d, J = 16.6 Hz, 1H), 5.77 (d, J = 10.4 Hz, 2H), 4.93 (s, 1H), 4.72 (dd, J = 11.7, 7.4 Hz, 1H), 4.47 (dd, J = 11.8, 3.5 Hz, 2H), 4.07 (d, J = 11.5 Hz, 3H), 3.95 (s, 2H), 3.87 (d, J = 14.9 Hz, 2H), 3.75 (s, 3H), 3.67 (d, J = 9.8 Hz, 1H), 3.29 (d, J = 79.0 Hz, 4H), 2.80 (m, 5H), 2.60 (dd, J = 16.7, 5.4 Hz, 1H), 2.12 (m, 4H). |
| 1-69 | 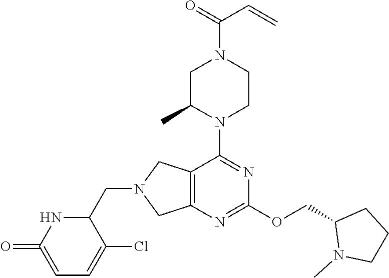 | H | M + 1 = 528.3 | 0%/ 0% | 1H NMR (400 MHz, CD₃OD) δ 7.59 (d, J = 9.6 Hz, 1H), 6.80 (td, J = 17.1, 10.5 Hz, 1H), 6.48 (d, J = 9.6 Hz, 1H), 6.28 (dd, J = 16.9, 4.5 Hz, 1H), 5.81 (dd, J = 10.6, 1.9 Hz, 1H), 4.74-4.08 (m, 7H), 4.03 (s, 3H), 3.89 (s, 2H), 3.72-3.45 (m, 4H), 3.18-2.97 (m, 2H), 2.91 (s, 3H), 2.37- 1.84 (m, 4H), 1.27- 1.20 (m, 3H). |
| 1-70 | 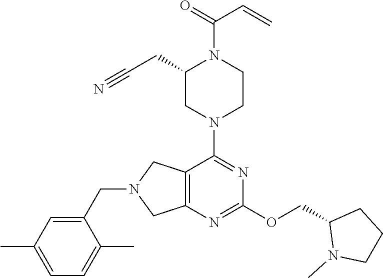 [SMILES: C=CC(=O)N1CCN(c2nc(OC[C@@H]3CCCN3C)nc3c2CN(Cc2cc(C)ccc2C)C3)C[C@@H]1CC#N] | A | M + 1 = 530.3 | 77%/ 94% | 1H NMR (400 MHz, CD₃OD) δ 8.53 (s, 1H), 7.16 (s, 1H), 7.06 (d, J = 7.7 Hz, 1H), 7.00 (d, J = 7.5 Hz, 1H), 6.79 (dd, J = 29.0, 17.2 Hz, 1H), 6.28 (d, J = 16.8 Hz, 1H), 5.82 (d, J = 9.9 Hz, 1H), 4.95 (d, J = 16.3 Hz, 1H), 4.77-4.64 (m, 2H), 4.45 (dd, J = 12.2, 7.3 Hz, 1H), 4.27-3.97 (m, 4H), 3.89 (s, 2H), 3.72 (s, 2H), 3.68-3.43 (m, 3H), 3.38 (d, J = 12.0 Hz, 2H), 3.05 (dd, J = 18.1, 8.5 Hz, 1H), 2.91 (d, J = 14.1 Hz, 4H), 2.77 (d, J = 17.7 Hz, 1H), 2.34 (s, 3H), 2.30 (s, 3H), 2.25 (d, J = 8.0 Hz, 1H), 2.06 (ddd, J = 20.5, 10.6, 4.3 Hz, 2H), 1.98-1.91 (m, 1H). |
| 1-71 | 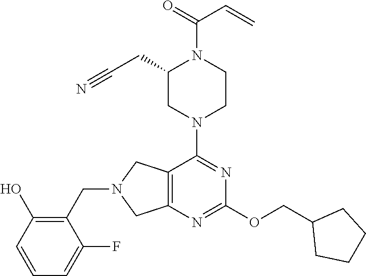 [SMILES: C=CC(=O)N1CCN(c2nc(OCC3CCCC3)nc3c2CN(Cc2c(O)cccc2F)C3)C[C@@H]1CC#N] | A | M + 1 = 521.3 | 9%/ 45% | 1H NMR (400 MHz, DMSO) δ 10.49 (s, 1H), 7.13 (dd, J = 15.2, 8.2 Hz, 1H), 6.90-6.76 (m, 1H), 6.64 (dd, J = 18.6, 8.6 Hz, 2H), 6.16 (dd, J = 16.6, 1.9 Hz, 1H), 5.75 (d, J = 10.3 Hz, 1H), 4.80 (d, J = 62.7 Hz, 1H), 4.30 (d, J = 13.6 Hz, 1H), 4.16-3.98 (m, 5H), 3.94 (s, 2H), 3.68 (s, 2H), 3.51-3.38 (m, 1H), 3.22 (dd, J = 26.4, 17.0 Hz, 2H), 3.02 (s, 1H), 2.87 (s, 2H), 2.25 (dt, J = 15.0, 7.5 Hz, 1H), 1.71 (dt, J = 11.7, 6.6 Hz, 2H), 1.61- 1.47 (m, 4H), 1.26 (dd, J = 12.3, 6.9 Hz, 2H). |
| 1-72 | 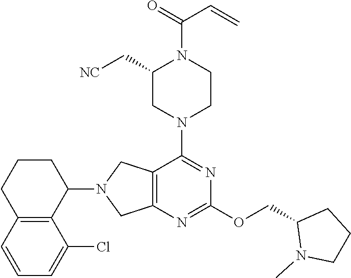 [SMILES: C=CC(=O)N1CCN(c2nc(OC[C@@H]3CCCN3C)nc3c2CN(C2CCCc4cccc(Cl)c42)C3)C[C@@H]1CC#N] | A | M + 1 = 576.3 | 95%/ 98% | 1H NMR (400 MHz, CD₃OD) δ 8.53 (s, 1H), 7.35- 7.04 (m, 3H), 6.76 (s, 1H), 6.27 (d, J = 16.6 Hz, 1H), 5.81 (d, J = 10.4 Hz, 1H), 4.94 (d, J = 13.7 Hz, 1H), 4.72 (d, J = 8.6 Hz, 2H), 4.54-4.32 (m, 3H), 4.06 (d, J = 11.2 Hz, 3H), 3.94 (dd, J = 14.4, 7.6 Hz, 1H), 3.73 (d, J = 5.4 Hz, 1H), 3.67-3.43 (m, 3H), 3.31- 3.35 (m, 2H), 3.13 (dd, J = 19.0, 8.1 Hz, 1H), 3.07- 2.94 (m, 4H), 2.91 (s, 1H), 2.82-2.67 (m, 2H), 2.43- 2.21 (m, 2H), 2.16-1.86 (m, 4H), 1.74 (d, J = 3.8 Hz, 1H), 1.58 (dd, J = 5.6, 2.7 Hz, 1H). |
| 1-73 | 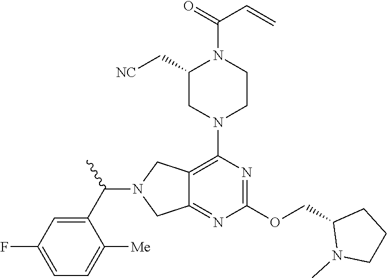 [SMILES: C=CC(=O)N1CCN(c2nc(OC[C@@H]3CCCN3C)nc3c2CN(C(C)c2cc(F)ccc2C)C3)C[C@@H]1CC#N] | A | M + 1 = 548.3 | 61%/ 86% | 1H NMR (400 MHz, CD₃OD) δ 7.25-7.14 (m, 2H), 6.90-6.69 (m, 2H), 6.26 (d, J = 16.5 Hz, 1H), 5.80 (d, J = 9.5 Hz, 1H), 4.97 (s, 1H), 4.77-4.43 (m, 2H), 4.34 (d, J = 6.0 Hz, 2H), 4.03 (dt, J = 11.3, 8.9 Hz, 4H), 3.68 (s, 2H), 3.56- 3.32 (m, 2H), 3.12 (ddd, J = 15.2, 9.1, 6.0 Hz, 2H), 2.94- 2.76 (m, 3H), 2.53 (s, 3H), 2.44-2.35 (m, 4H), 2.09 (ddd, J = 16.4, 12.5, 8.3 Hz, 1H), 1.87-1.78 (m, 2H), 1.70 (dt, J = 19.9, 7.3 Hz, 1H), 1.39 (d, J = 6.4 Hz, 3H). |
| 1-74 | 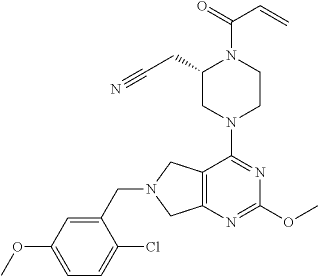 | A | M + 1 = 483.2 | 2%/ 21% | 1H NMR (400 MHz, CD₃OD) δ 7.37 (d, 1H), 7.29 (s, 1H), 6.86-6.83 (m, 2H), 6.29 (d, 1H), 5.83 (d, 1H), 5.04 (br, 1H), 4.65-4.61 (m, 2H), 4.19 (s, 4H), 4.02 (s, 3H), 3.93 (s, 3H), 379 (s, 4H), 3.32-3.30 (m, 1H) 2.92-2.86 (m, 3H). |
| 1-75 | 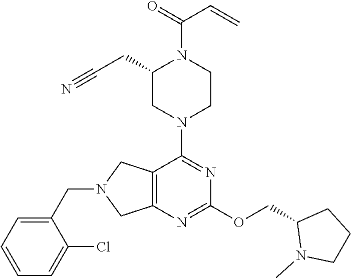 [SMILES: C=CC(=O)N1CCN(c2nc(OC[C@@H]3CCCN3C)nc3c2CN(Cc2ccccc2Cl)C3)C[C@@H]1CC#N] | A | M + 1 = 536.3 | 96%/ 97% | 1H NMR (400 MHz, CD₃OD) δ 7.58-7.53 (m, 1H), 7.42 (d, J = 7.5 Hz, 1H), 7.35- 7.25 (m, 2H), 6.78 (s, 1H), 6.27 (d, J = 16.8 Hz, 1H), 5.82 (d, J = 10.1 Hz, 1H), 4.94 (s, 1H), 4.74-4.63 (m, 1H), 4.63 (s, 1H), 4.45-4.38 (m, 1H), 4.27-3.98 (m, 6H), 3.81 (s, 2H), 3.63-3.34 (m, 4H), 3.25- 3.04 (m, 1H), 3.01- 2.76 (m, J = 26.3 Hz, 6H), 2.31-2.17 (m, 1H), 1.92- 1.80 (m, 3H). |
| 1-76 | 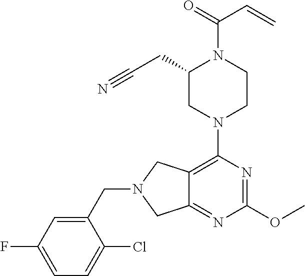 [SMILES: C=CC(=O)N1CCN(c2nc(OC)nc3c2CN(Cc2cc(F)ccc2Cl)C3)C[C@@H]1CC#N] | A | M + 1 = 471.1 | 0%/ 7% | 1H NMR (400 MHz, CD₃OD) δ 7.38 (ddd, J = 12.1, 9.0, 3.9 Hz, 2H), 7.04 (td, J = 8.4, 2.9 Hz, 1H), 6.79 (d, J = 12.6 Hz, 1H), 6.27 (d, J = 16.5 Hz, 1H), 5.81 (d, J = 10.1 Hz, 1H), 5.01 (s, 1H), 4.63 (d, J = 13.8 Hz, 2H), 4.19 (s, 3H), 3.99 (d, J = 44.0 Hz, 5H), 3.80 (s, 2H), 3.47 (d, J = 58.1 Hz, 2H), 2.89 (dd, J = 16.7, 7.3 Hz, 3H). |
| 1-77 | 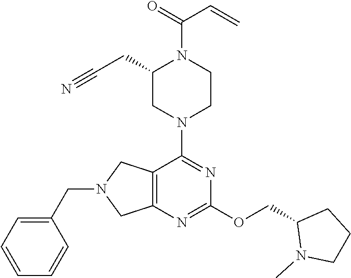 [SMILES: C=CC(=O)N1CCN(c2nc(OC[C@@H]3CCCN3C)nc3c2CN(Cc2ccccc2)C3)C[C@@H]1CC#N] | A | M + 1 = 502.3 | 27%/ 43% | 1H NMR (400 MHz, CDCl₃) δ 8.06 (s, 0H), 7.53 (d, J = 11.0 Hz, 2H), 7.46-7.30 (m, 3H), 6.52 (s, 1H), 6.37 (d, J = 16.2 Hz, 1H), 5.81 (d, J = 10.3 Hz, 1H), 5.18 (s, 1H), 4.94 (s, 1H), 4.61 (d, J = 9.4 Hz, 1H), 4.48 (s, 2H), 4.18 (s, 3H), 4.03 (s, 2H), 3.91 (s, 2H), 3.57 (s, 2H), 3.45 (d, J = 11.1 Hz, 1H), 3.25 (s, 1H), 3.00 (s, 3H), 2.89 (s, 2H), 2.63 (dd, J = 16.7, 4.6 Hz, 1H), 2.31 (s, 2H), 2.21- 1.97 (m, 3H). |
| 1-78 | 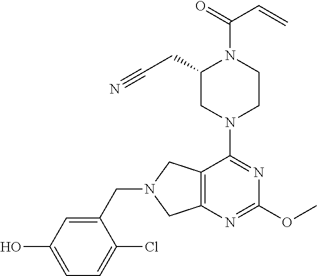 [SMILES: C=CC(=O)N1CCN(c2nc(OC)nc3c2CN(Cc2cc(O)ccc2Cl)C3)C[C@@H]1CC#N] | A | M + 1 = 469.2 | 89%/ 97% | 1H NMR (400 MHz, CD₃OD) δ 8.24 (s, 0.76H), 7.19 (d, J = 8.7 Hz, 1H), 6.97 (d, J = 2.9 Hz, 1H), 6.89- 6.67 (m, 2H), 6.27 (d, J = 16.8 Hz, 1H), 5.81 (d, J = 10.3 Hz, 1H), 5.01 (s, 1H), 4.79-4.48 (m, 2H), 4.18 (s, 3H), 4.06 (d, J = 12.4 Hz, 1H), 3.97 (s, 2H), 3.93 (s, 3H), 3.79 (s, 2H), 3.52 (dd, J = 33.9, 11.3 Hz, 1H), 3.42-3.31 (m, 1H), 2.85 (s, 2H). |
| 1-79 | 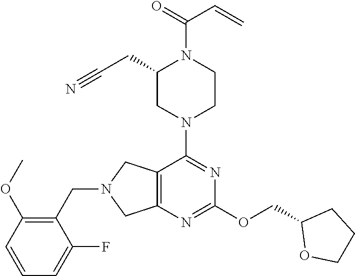 [SMILES: C=CC(=O)N1CCN(c2nc(OC[C@@H]3CCCO3)nc3c2CN(Cc2c(F)cccc2OC)C3)C[C@@H]1CC#N] | A | M + 1 = 537.3 | 3%/ 23% | 1H NMR (400 MHz, CD₃OD) δ 7.31 (dd, J = 15.2, 8.4 Hz, 1H), 7.00-6.70 (m, 3H), 6.27 (d, J = 17.0 Hz, 1H), 5.82 (d, J = 10.2 Hz, 1H), 5.00 (s, 1H), 4.79- 4.43 (m, 2H), 4.28 (tdd, J = 12.6, 10.5, 4.9 Hz, 3H), 4.17 (d, J = 14.3 Hz, 3H), 3.99 (s, 2H), 3.89 (d, J = 4.2 Hz, 3H), 3.85-3.71 (m, 3H), 3.51 (d, J = 23.6 Hz, 1H), 3.34 (d, J = 9.8 Hz, 2H), 3.22-3.04 (m, 1H), 2.87 (dt, J = 15.1, 7.6 Hz, 2H), 1.98 (dtdd, J = 14.6, 11.7, 7.6, 5.1 Hz, 3H), 1.84-1.72 (m, 1H). |
| 1-80 | 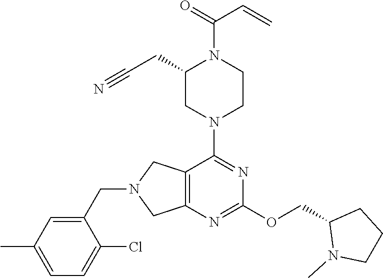 [SMILES: C=CC(=O)N1CCN(c2nc(OC[C@@H]3CCCN3C)nc3c2CN(Cc2cc(C)ccc2Cl)C3)C[C@@H]1CC#N] | A | M + 1 = 550.3 | 94%/ 96% | 1H NMR (400 MHz, CD₃OD) δ 7.44-7.21 (m, 2H), 7.09 (d, J = 8.1 Hz, 1H), 6.77 (s, 1H), 6.27 (d, J = 16.2 Hz, 1H), 5.82 (d, J = 9.8 Hz, 1H), 4.99 (s, 1H), 4.61 (d, J = 14.1 Hz, 2H), 4.34 (d, J = 5.9 Hz, 2H), 4.10 (d, J = 66.7 Hz, 5H), 3.78 (s, 2H), 3.50 (d, J = 22.0 Hz, 2H), 3.23-3.03 (m, 2H), 2.99-2.73 (m, 3H), 2.62-2.29 (m, 7H), 2.09 (dt, J = 16.6, 8.5 Hz, 1H), 1.75 (ddd, J = 26.8, 12.0, 7.1 Hz, 3H). |
| 1-81 | 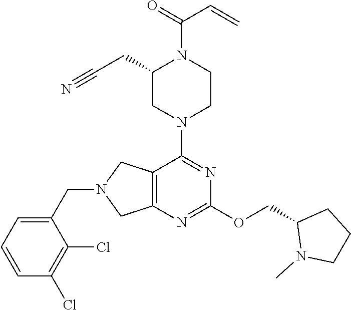 [SMILES: C=CC(=O)N1CCN(c2nc(OC[C@@H]3CCCN3C)nc3c2CN(Cc2cccc(Cl)c2Cl)C3)C[C@@H]1CC#N] | A | M + 1 = 570.2 | 96%/ 98% | 1H NMR (400 MHz, CDCl₃) δ 7.41 (t, J = 6.9 Hz, 2H), 7.22 (t, J = 7.8 Hz, 2H), 6.54 (s, 1H), 6.38 (d, J = 16.7 Hz, 1H), 5.82 (d, J = 10.7 Hz, 1H), 4.98 (s, 1H), 4.58 (s, 1H), 4.30 (s, 1H), 4.13 (dd, J = 21.0, 11.0 Hz, 2H), 4.06 (s, 2H), 3.87 (t, J = 8.0 Hz, 3H), 3.42 (s, 2H), 3.24 (d, J = 39.1 Hz, 2H), 2.80 (dd, J = 16.8, 8.4 Hz, 1H), 2.64 (d, J = 11.9 Hz, 5H), 2.13 (s, 1H), 1.85 (s, 4H), 1.65 (s, 2H). |
| 1-82 | 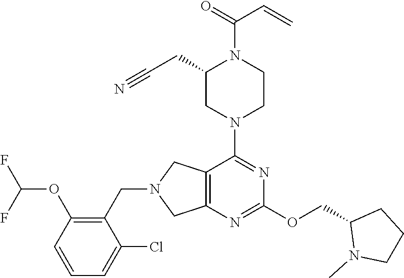 [SMILES: C=CC(=O)N1CCN(c2nc(OC[C@@H]3CCCN3C)nc3c2CN(Cc2c(Cl)cccc2OC(F)F)C3)C[C@@H]1CC#N] | A | M + 1 = 602.2 | 94%/ 98% | 1H NMR (400 MHz, CD₃OD) δ 7.36 (dd, J = 7.6, 6.1 Hz, 2H), 7.21-7.14 (m, 1H), 7.11-6.72 (m, 2H), 6.27 (d, J = 16.6 Hz, 1H), 5.82 (d, J = 10.4 Hz, 1H), 4.99 (s, 1H), 4.80-4.46 (m, 2H), 4.33 (d, J = 5.6 Hz, 2H), 4.22 (s, 2H), 4.13 (s, 2H), 4.06 (d, J = 20.8 Hz, 1H), 3.81 (s, 2H), 3.51 (d, J = 25.3 Hz, 1H), 3.38 (d, J = 13.8 Hz, 1H), 3.21- 3.03 (m, 2H), 2.83 (ddd, J = 20.2, 14.9, 6.9 Hz, 3H), 2.51 (s, 3H), 2.38 (dd, J = 17.9, 9.0 Hz, 1H), 2.12- 2.02 (m, 1H), 1.86-1.76 (m, 2H), 1.74-1.64 (m, 1H). |
| 1-83 | 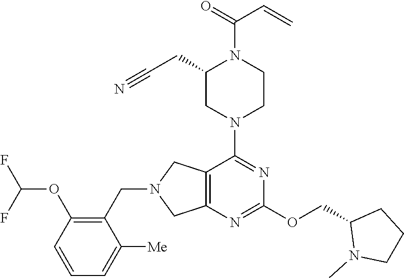 [SMILES: C=CC(=O)N1CCN(c2nc(OC[C@@H]3CCCN3C)nc3c2CN(Cc2c(C)cccc2OC(F)F)C3)C[C@@H]1CC#N] | A | M + 1 = 582.30 | 97%/ 100% | 1H NMR (400 MHz, CD₃OD) δ 8.54 (s, 0.2 H), 7.23- 7.09 (m, 1H), 7.03 (d, 1H), 7.03-6.66 (m, 3H), 6.30 (d, 1H), 5.83 (d, 1H), 5.02 (br, 1H), 4.62-4.35 (m, 2H), 4.32-4.20 (m, 2H), 4.24-4.23 (m, 3H), 4.0 (s, 2H), 3.73 (s, 2H), 3.52-3.32 (m, 2H), 3.23-3.18 (m, 2H), 2.96-2.80 (m, 3H), 2.59 (s, 3H), 2.57-2.48 (m, 1H), 2.47 (s, 3H), 2.19- 2.08 (m, 1H), 1.88-1.84 (m, 3H). |
| 1-84 | 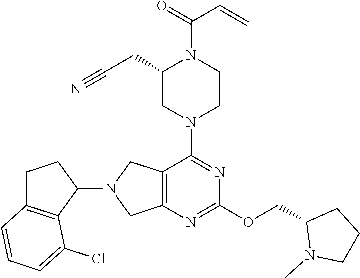 [SMILES: C=CC(=O)N1CCN(c2nc(OC[C@@H]3CCCN3C)nc3c2CN(C2CCc4cccc(Cl)c42)C3)C[C@@H]1CC#N] | A | M + 1 = 562.2 | 98%/ 99% | 1H NMR (400 MHz, CD₃OD) δ 8.54 (s, 1H), 7.37- 7.09 (m, 3H), 6.78 (s, 1H), 6.27 (d, J = 16.5 Hz, 1H), 5.83 (s, 1H), 4.97 (s, 1H), 4.62 (d, J = 7.0 Hz, 3H), 4.54 (s, 1H), 4.40 (d, J = 6.8 Hz, 1H), 4.31-4.13 (m, 3H), 4.08 (s, 1H), 3.80 (d, J = 13.7 Hz, 2H), 3.52 (s, 1H), 3.36 (s, 2H), 3.23 (dd, J = 17.1, 8.9 Hz, 3H), 2.91 (dd, J = 15.5, 8.0 Hz, 2H), 2.75 (s, 4H), 2.35 (dd, J = 10.7, 8.9 Hz, 1H), 2.21 (dd, J = 10.3, 5.1 Hz, 2H), 1.96 (d, J = 8.1 Hz, 3H) |
| 1-85 | 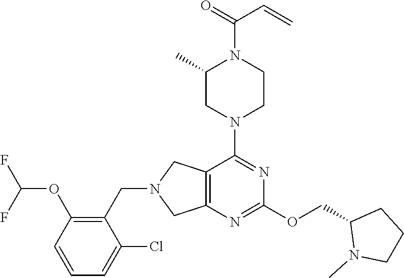 [SMILES: C=CC(=O)N1CCN(c2nc(OC[C@@H]3CCCN3C)nc3c2CN(Cc2c(Cl)cccc2OC(F)F)C3)C[C@@H]1C] | B | M + 1 = 577.2 | 0%/ 0% | 1H NMR (400 MHz, CD₃OD) δ 7.39-7.33 (m, 2H), 7.19 (dd, J = 9.2, 4.4 Hz, 1H), 7.12-6.73 (m, 2H), 6.23 (d, J = 16.6 Hz, 1H), 5.77 (d, J = 10.7 Hz, 1H), 4.69 (d, J = 69.4 Hz, 1H), 4.39-4.28 (m, 3H), 4.20 (t, J = 6.6 Hz, 3H), 4.13 (s, 2H), 4.01 (s, 1H), 3.81 (s, 2H), 3.52 (s, 1H), 3.37 (d, J = 13.2 Hz, 1H), 3.20 (s, 1H), 3.12-3.06 (m, 1H), 2.77 (d, J = 5.0 Hz, 1H), 2.51 (d, J = 1.3 Hz, 3H), 2.42-2.34 (m, 1H), 2.12- 2.03 (m, 1H), 1.85-1.76 (m, 2H), 1.73-1.64 (m, 1H), 1.22 (s, 3H). |
| 1-86 | 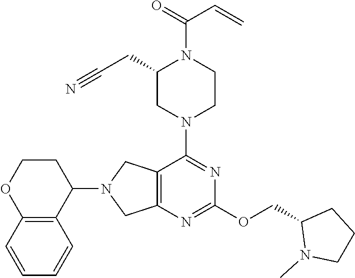 [SMILES: C=CC(=O)N1CCN(c2nc(OC[C@@H]3CCCN3C)nc3c2CN(C2CCOc4ccccc42)C3)C[C@@H]1CC#N] | A | M + 1 = 544.3 | 59%/ 93% | 1H NMR (400 MHz, CD₃OD) δ 7.32 (d, J = 7.5 Hz, 1H), 7.15 (t, J = 7.7 Hz, 1H), 6.91-6.72 (m, 3H), 6.27 (d, J = 16.7 Hz, 1H), 5.81 (d, J = 10.2 Hz, 1H), 4.98 (s, 1H), 4.64 (dd, J = 40.9, 28.2 Hz, 2H), 4.47- 4.28 (m, 4H), 4.21 (d, J = 10.3 Hz, 2H), 4.02 (d, J = 11.4 Hz, 2H), 3.92 (d, J = 13.3 Hz, 2H), 3.66 (d, J = 14.3 Hz, 1H), 3.36 (s, 1H), 3.13 (d, J = 4.8 Hz, 2H), 2.90 (d, J = 27.9 Hz, 3H), 2.55 (s, 3H), 2.45 (d, J = 8.3 Hz, 1H), 2.26 (d, J = 14.2 Hz, 1H), 2.16-1.99 (m, 2H), 1.77 (ddd, J = 19.5, 13.8, 7.3 Hz, 3H). |
| 1-87 | 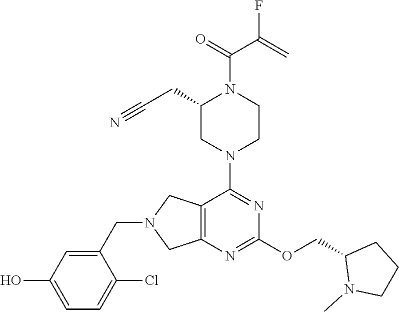 [SMILES: C=C(F)C(=O)N1CCN(c2nc(OC[C@@H]3CCCN3C)nc3c2CN(Cc2cc(O)ccc2Cl)C3)C[C@@H]1CC#N] | A | M + 1 = 570.2 | 97%/ 99% | 1H NMR (400 MHz, DMSO) δ 7.18 (d, J = 8.6 Hz, 1H), 6.95 (d, J = 2.8 Hz, 1H), 6.66 (dd, J = 8.6, 2.9 Hz, 1H), 5.34 (s, 1H), 5.24 (d, J = 52.1 Hz, 1H), 4.78 (s, 1H), 4.31 (d, J = 13.5 Hz, 1H), 4.18 (s, 1H), 4.05 (dd, J = 14.6, 8.3 Hz, 4H), 3.86 (s, 2H), 3.70 (s, 2H), 3.25-2.83 (m, 8H), 2.31 (s, 3H), 2.14 (d, J = 8.4 Hz, 1H), 1.96-1.86 (m, 1H), 1.63 (s, 2H), 1.59-1.49 (m, 1H). |
| 1-88 | 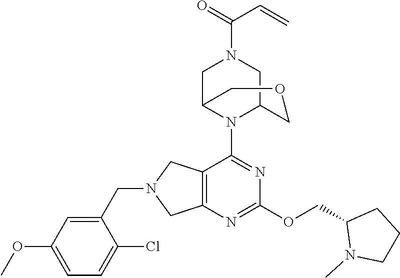 [SMILES: C=CC(=O)N1CC2COCC(C1)N2c1nc(OC[C@@H]2CCCN2C)nc2c1CN(Cc1cc(OC)ccc1Cl)C2] | A | M + 1 = 569.2 | 0%/ 0% | Intermediate. No NMR |
| 1-90 | 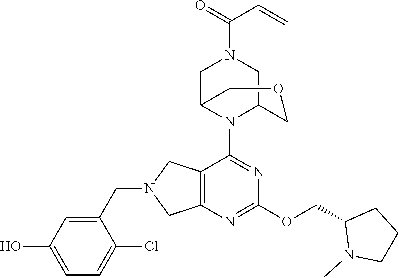 [SMILES: C=CC(=O)N1CC2COCC(C1)N2c1nc(OC[C@@H]2CCCN2C)nc2c1CN(Cc1cc(O)ccc1Cl)C2] | A | M + 1 = 555.2 | 2%/ 12% | 1H NMR (400 MHz, CD₃OD) δ 7.20 (d, J = 8.6 Hz, 1H), 6.98 (d, J = 2.9 Hz, 1H), 6.79 (dd, J = 16.8, 10.7 Hz, 1H), 6.70 (dd, J = 8.6, 3.0 Hz, 1H), 6.19 (dd, J = 16.8, 1.9 Hz, 1H), 5.76 (dd, J = 10.7, 2.0 Hz, 1H), 4.79 (dd, J = 13.6, 1.7 Hz, 1H), 4.61-4.30 (m, 4H), 4.10 (t, J = 2.1 Hz, 2H), 4.03 (dd, J = 11.5, 5.1 Hz, 2H), 3.99 (s, 2H), 3.87 (dq, J = 5.1, 2.4 Hz, 1H), 3.82 (q, J = 2.9, 2.0 Hz, 3H), 3.64 (dt, J = 13.5, 3.1 Hz, 1H), 3.50-3.37 (m, 2H), 3.28-3.13 (m, 2H), 2.97- 2.87 (m, 1H), 2.83 (s, 3H), 2.33-1.79 (m, 4H). |
| 1-91 | 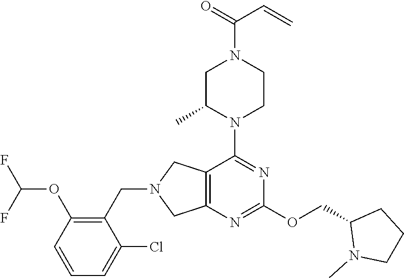 [SMILES: C=CC(=O)N1CCN(c2nc(OC[C@@H]3CCCN3C)nc3c2CN(Cc2c(Cl)cccc2OC(F)F)C3)[C@H](C)C1] | G | M + 1 = 577.3 | 0%/ 0% | 1H NMR (400 MHz, CD₃OD) δ 7.39-7.32 (m, 2H), 7.18 (dd, J = 5.2, 4.2 Hz, 1H), 7.12-6.73 (m, 2H), 6.27 (dd, J = 16.8, 3.8 Hz, 1H), 5.80 (dd, J = 10.6, 1.9 Hz, 1H), 4.62 (s, 1H), 4.43 (dd, J = 40.9, 13.3 Hz, 1H), 4.34-4.26 (m, 2H), 4.25- 4.15 (m, 3H), 4.12 (s, 2H), 4.00 (d, J = 13.8 Hz, 1H), 3.80 (s, 2H), 3.62- 3.49 (m, 1H), 3.37 (d, J = 9.2 Hz, 1H), 3.18-2.98 (m, 2H), 2.75 (dt, J = 13.7, 6.6 Hz, 1H), 2.49 (s, 3H), 2.36 (dd, J = 17.9, 9.1 Hz, 1H), 2.08 (ddd, J = 16.1, 12.5, 8.3 Hz, 1H), 1.85- 1.76 (m, 2H), 1.72-1.63 (m, 1H), 1.21 (d, J = 4.9 Hz, 3H). |
| 1-92 | 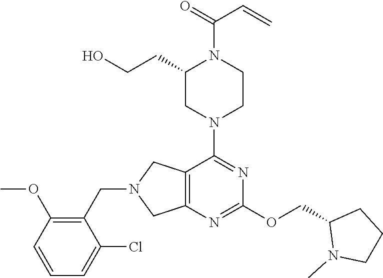 [SMILES: C=CC(=O)N1CCN(c2nc(OC[C@@H]3CCCN3C)nc3c2CN(Cc2c(Cl)cccc2OC)C3)C[C@@H]1CCO] | G | M + 1 = 571.2 | 0%/ 0% | 1H NMR (400 MHz, CD₃OD) δ 8.43 (s, 1H), 7.28 (t, J = 8.2 Hz, 1H), 7.14-6.95 (m, 2H), 6.95-6.71 (m, 1H), 6.25 (dd, J = 16.2, 8.5 Hz, 1H), 5.91-5.68 (m, 1H), 4.83 (s, 1H), 4.62 (d, J = 3.2 Hz, 1H), 4.49 (dd, J= 12.5, 7.3 Hz, 3H), 4.22 (s, 3H), 4.14 (s, 2H), 4.00 (dd, J = 38.1, 13.2 Hz, 1H), 3.89 (s, 3H), 3.83 (s, 2H), 3.80-3.73 (m, 1H), 3.65 (s, 1H), 3.55 (s, 2H), 3.34 (s, 1H), 3.29-3.03 (m, 3H), 3.00 (s, 3H), 2.40- 2.30 (m, 1H), 2.22-1.93 (m, 3H), 1.81 (s, 2H). |
| 1-93 | 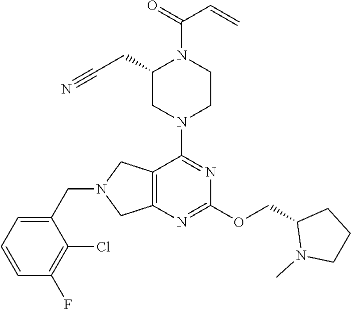 | A | M + 1 = 584.2 | 96%/ 97% | 1H NMR (400 MHz, CD₃OD) δ 7.40-7.36 (m, 2H), 7.31-7.20 (m, 1H), 6.77-6.75 (m, 1H), 6.29 (d, 1H), 5.83 (d, 1H), 4.87 (br, 1H), 4.62-4.51 (m, 2H), 4.34 (d, 2H), 4.19 (s, 3H), 4.08 (s, 2H), 3.79 (s, 2H), 3.37- 3.30 (m, 2H), 3.10-3.08 (m, 2H), 2.9-2.79 (m, 3H), 2.50 (s, 3H), 2.35-2.32 (m, 1H), 2.06-2.03 (m, 1H), 1.51-1.21 (m, 3H). |
| 1-94 | 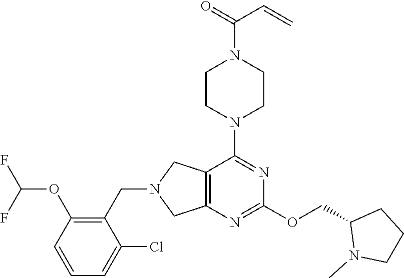 [SMILES: C=CC(=O)N1CCN(c2nc(OC[C@@H]3CCCN3C)nc3c2CN(Cc2c(Cl)cccc2OC(F)F)C3)CC1] | G | M + 1 = 563.2 | 0%/ 0% | 1H NMR (400 MHz, CDCl₃) δ 8.70 (s, 5H), 7.48 (dd, J = 19.6, 11.3 Hz, 1H), 7.36 (d, J = 8.2 Hz, 1H), 7.19 (d, J = 8.2 Hz, 1H), 7.00- 6.74 (m, 1H), 6.58-6.50 (m, 1H), 6.35 (dd, J = 16.8, 1.5 Hz, 1H), 5.81 (dd, J = 10.5, 1.5 Hz, 1H), 4.97 (d, J = 12.8 Hz, 1H), 4.91- 4.77 (m, 2H), 4.63 (s, 2H), 4.61-4.47 (m, 3H), 3.96 (s, 1H), 3.76 (d, J = 37.0 Hz, 9H), 3.08 (s, 3H), 3.04- 2.91 (m, 1H), 2.33 (ddd, J = 23.6, 15.0, 7.0 Hz, 2H), 2.15 (dt, J = 17.0, 7.0 Hz, 2H). |
| 1-95 | 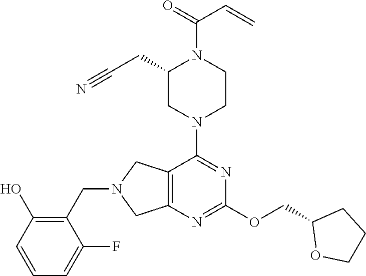 [SMILES: C=CC(=O)N1CCN(c2nc(OC[C@@H]3CCCO3)nc3c2CN(Cc2c(O)cccc2F)C3)C[C@@H]1CC#N] | A | M + 1 = 523.2 | 21%/ 70% | 1H NMR (400 MHz, CD₃OD) δ 7.19-7.06 (m, 1H), 6.96-6.69 (m, 1H), 6.67- 6.54 (m, 2H), 6.27 (d, J = 16.8 Hz, 1H), 5.81 (d, J = 10.1 Hz, 1H), 5.00 (s, 1H), 4.77-4.42 (m, 2H), 4.38-4.01 (m, 8H), 3.87 (dt, J = 13.4, 6.7 Hz, 1H), 3.83-3.72 (m, 3H), 3.51 (d, J = 22.5 Hz, 1H), 3.37 (d, J = 17.7 Hz, 1H), 3.13 (s, 1H), 3.01-2.75 (m, 2H), 2.12- 1.71 (m, 4H). |
| 1-96 | 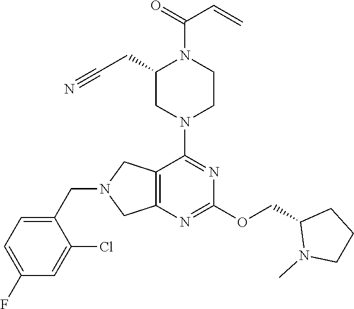 [SMILES: C=CC(=O)N1CCN(c2nc(OC[C@@H]3CCCN3C)nc3c2CN(Cc2ccc(F)cc2Cl)C3)C[C@@H]1CC#N] | A | M + 1 = 554.2 | 51%/ 93% | 1H NMR (400 MHz, CD₃OD) δ 7.64-7.48 (m, 1H), 7.25 (dd, J = 8.7, 2.5 Hz, 1H), 7.10 (td, J = 8.3, 2.5 Hz, 1H), 6.77 (s, 1H), 6.27 (d, J = 16.4 Hz, 1H), 5.81 (d, J = 9.4 Hz, 1H), 4.99 (s, 1H), 4.65 (d, J = 12.7 Hz, 1H), 4.40 (dd, J = 25.2, 18.4 Hz, 2H), 4.10 (d, J = 59.1 Hz, 6H), 3.78 (s, 2H), 3.48 (s, 3H), 2.94 (dd, J = 71.1, 54.9 Hz, 4H), 2.67 (s, 4H), 2.15 (dd, J = 18.4, 10.1 Hz, 1H), 1.79 (s, 3H). |
| 1-97 | 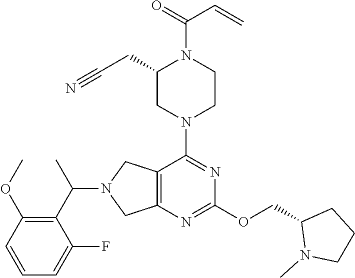 [SMILES: C=CC(=O)N1CCN(c2nc(OC[C@@H]3CCCN3C)nc3c2CN(C(C)c2c(F)cccc2OC)C3)C[C@@H]1CC#N] | A | M + 1 = 564.3 | 95%/ 98% | 1H NMR (400 MHz, CD₃OD) δ 8.45 (s, 1H), 7.32 (dt, J = 14.9, 7.5 Hz, 1H), 6.88 (d, J = 8.4 Hz, 1H), 6.78-6.71 (m, 1H), 6.27 (d, J = 16.6 Hz, 1H), 5.82 (d, J = 9.8 Hz, 1H), 4.73 (s, 3H), 4.60-4.42 (m, 3H), 4.31 (d, J = 10.9 Hz, 1H), 4.06 (d, J = 10.5 Hz, 3H), 3.88 (s, 3H), 3.82 (d, J = 14.2 Hz, 2H), 3.69 (d, J = 14.5 Hz, 2H), 3.55 (d, J = 34.6 Hz, 1H), 3.36 (s, 1H), 3.19 (dd, J = 18.4, 7.7 Hz, 1H), 3.01 (s, 3H), 2.90 (s, 1H), 2.78 (s, 1H), 2.34 (dt, J = 14.3, 7.1 Hz, 1H), 2.05 (ddd, J = 19.0, 17.3, 10.1 Hz, 3H), 1.60 (d, J = 6.6 Hz, 3H). |
| 1-98 | 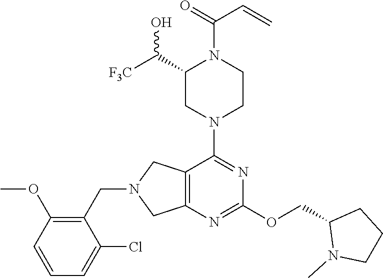 [SMILES: C=CC(=O)N1CCN(c2nc(OC[C@@H]3CCCN3C)nc3c2CN(Cc2c(Cl)cccc2OC)C3)C[C@@H]1C(O)C(F)(F)F] | B | M + 1 = 625.2 | 0%/ 1% | 1H NMR (400 MHz, CD₃OD) δ 8.47 (s, 1H), 7.29 (t, J = 8.2 Hz, 1H), 7.03 (dd, J = 18.5, 8.2 Hz, 2H), 6.76 (ddd, J = 19.2, 17.1, 10.8 Hz, 1H), 6.22 (dd, J = 16.6, 9.2 Hz,_1H), 5.78 (dd, J = 20.2, 10.8 Hz, 1H), 5.05 (d, J = 2.5 Hz, 1H), 4.65- 4.54 (m, 2H), 4.50 (dd, J = 12.2, 7.7 Hz, 2H), 4.23 (d, J = 11.0 Hz, 3H), 4.14 (s, 2H), 4.13-4.02 (m, 1H), 3.89 (s, 3H), 3.85 (s, 2H), 3.82-3.76 (m, 1H), 3.74-3.50 (m, 2H), 3.40 (dd, J = 20.2, 12.8 Hz, 1H), 3.28-3.12 (m, 2H), 3.00 (s, 3H), 2.35 (td, J = 14.6, 8.1 Hz, 1H), 2.20-2.04 (m, 2H), 2.00-1.91 (m, 1H). |
| 1-99 | 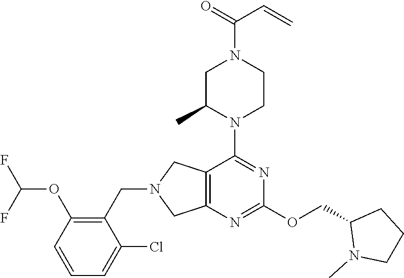 [SMILES: C=CC(=O)N1CCN(c2nc(OC[C@@H]3CCCN3C)nc3c2CN(Cc2c(Cl)cccc2OC(F)F)C3)[C@@H](C)C1] | G | M + 1 = 577.3 | 0%/ 6% | 1H NMR (400 MHz, CD₃OD) δ 8.55 (s, 1H), 7.37 (dd, J = 8.1, 5.8 Hz, 2H), 7.24-7.16 (m, 1H), 7.12- 6.75 (m, 2H), 6.28 (d, J = 16.7 Hz, 1H), 5.81 (d, J = 10.5 Hz, 1H), 4.68-4.53 (m, 2H), 4.52-4.37 (m, 2H), 4.22 (q, J = 11.4 Hz, 3H), 4.14 (s, 2H), 4.01 (d, J = 12.7 Hz, 1H), 3.83 (s, 2H), 3.56 (d, J = 11.6 Hz, 2H), 3.39 (d, J = 9.0 Hz, 2H), 3.17 (d, J = 14.4 Hz, 1H), 3.03 (d, J = 8.5 Hz, 1H), 2.90 (s, 3H), 2.30 (dt, J = 15.2, 8.1 Hz, 1H), 2.07 (ddd, J = 22.2, 12.9, 6.7 Hz, 2H), 1.97-1.86 (m, 1H), 1.22 (d, J = 5.7 Hz, 3H). |
| 1-100 | 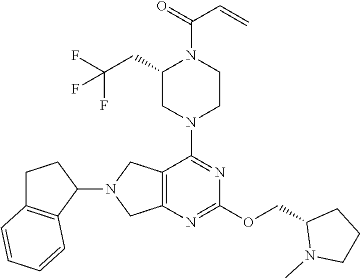 [SMILES: C=CC(=O)N1CCN(c2nc(OC[C@@H]3CCCN3C)nc3c2CN(C2CCc4ccccc42)C3)C[C@@H]1CC(F)(F)F] | B | M + 1 = 571.3 | 0%/ 5% | 1H NMR (400 MHz, CD₃OD) δ 7.65 (s, 1H), 7.37 (d, J = 30.0 Hz, 3H), 6.74 (s, 1H), 6.23 (d, J = 15.7 Hz, 1H), 5.80 (d, J = 9.7 Hz, 1H), 5.15 (s, 1H), 4.71 (s, 2H), 4.55 (s, 3H), 3.98 (d, J = 64.9 Hz, 3H), 3.75 (s, 1H), 3.58 (s, 1H), 3.20 (dd, J = 14.4, 7.2 Hz, 5H), 3.08 (s, 5H), 2.57 (s, 3H), 2.44 (d, J = 37.3 Hz, 2H), 2.11 (d, J = 68.9 Hz, 4H). |
| 1-101 | 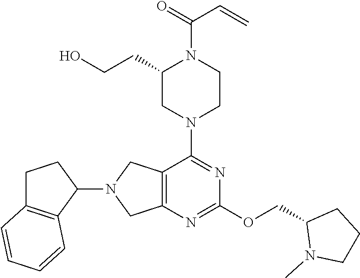 [SMILES: C=CC(=O)N1CCN(c2nc(OC[C@@H]3CCCN3C)nc3c2CN(C2CCc4ccccc42)C3)C[C@@H]1CCO] | B | M + 1 = 533.3 | 0%/ 1% | 1H NMR (400 MHz, CD₃OD) δ 8.38 (s, 3H), 7.43 (d, J = 7.2 Hz, 1H), 7.25 (tt, J = 14.5, 7.1 Hz, 3H), 6.95- 6.69 (m, 1H), 6.35-6.17 (m, 1H), 5.79 (d, J = 10.6 Hz, 1H), 4.66 (dd, J = 12.5, 3.1 Hz, 0H), 4.59-4.53 (m, 0H), 4.49 (dd, J = 12.5, 7.2 Hz, 1H), 4.34-4.16 (m, 1H), 4.04 (d, J = 13.3 Hz, 0H), 3.85 (d, J = 9.3 Hz, 1H), 3.83-3.76 (m, 0H), 3.72-3.41 (m, 1H), 3.35 (s, 0H), 3.25-3.06 (m, 1H), 3.02 (s, 1H), 2.94- 2.79 (m, 0H), 2.40-1.96 (m, 2H), 1.82 (s, 1H). |
| 1-102 | 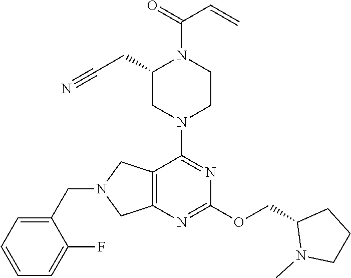 [SMILES: C=CC(=O)N1CCN(c2nc(OC[C@@H]3CCCN3C)nc3c2CN(Cc2ccccc2F)C3)C[C@@H]1CC#N] | A | M + 1 = 520.3 | 89%/ 96% | 1H NMR (400 MHz, CD₃OD) δ 7.47 (td, J = 7.5, 1.7 Hz, 1H), 7.32 (tdd, J = 7.3, 5.3, 1.8 Hz, 1H), 7.17 (td, J = 7.5, 1.0 Hz, 1H), 7.12- 7.06 (m, 1H), 6.77 (d, J = 11.1 Hz, 1H), 6.26 (d, J = 16.8 Hz, 1H), 5.80 (d, J = 10.1 Hz, 1H), 4.97 (s, 1H), 4.65 (t, J = 27.4 Hz, 2H), 4.43-4.30 (m, 2H), 4.15 (s, 3H), 3.97 (s, 2H), 3.74 (s, 2H), 3.43 (dd, J = 50.3, 21.5 Hz, 2H), 3.24-3.06 (m, 2H), 2.88 (dt, J = 22.7, 15.3 Hz, 3H), 2.59 (s, 3H), 2.52 (d, J = 8.2 Hz, 1H), 2.17-2.06 (m, 1H), 1.91-1.82 (m, 2H), 1.73 (dt, J = 14.2, 7.4 Hz, 1H). |
| 1-104 | 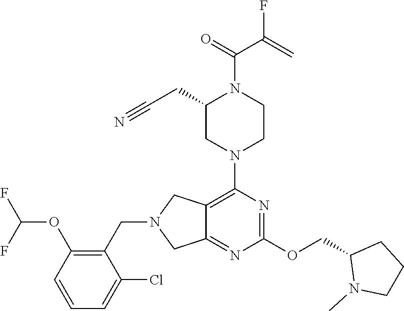 [SMILES: C=C(F)C(=O)N1CCN(c2nc(OC[C@@H]3CCCN3C)nc3c2CN(Cc2c(Cl)cccc2OC(F)F)C3)C[C@@H]1CC#N] | A | M + 1 = 620.3 | 8%/ 48% | 1H NMR (400 MHz, CD₃OD) δ 7.40-7.32 (m, 2H), 7.21-7.16 (m, 1H), 6.92 (t, J = 73.9 Hz, 1H) 5.31 (dt, J = 14.2, 12.5 Hz, 2H), 4.63 (d, J = 13.8 Hz, 1H), 4.36-4.29 (m, 2H), 4.21 (s, 2H), 4.17 (s, 1H), 4.13 (s, 2H), 4.03 (s, 1H), 3.81 (s, 2H), 3.55 (s, 1H), 3.38 (s, 2H), 3.23 (s, 1H), 3.12- 3.06 (m, 1H), 2.93 (d, J = 6.6 Hz, 2H), 2.82-2.74 (m, 1H), 2.51 (s, 3H), 2.38 (dd, J = 17.9, 9.0 Hz, 1H), 2.12-2.03 (m, 1H), 1.82 (dd, J = 7.7, 5.0 Hz, 2H), 1.73-1.64 (m, 1H). |
| 1-105 | 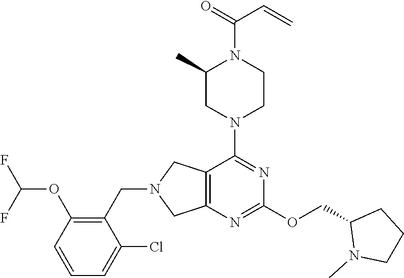 [SMILES: C=CC(=O)N1CCN(c2nc(OC[C@@H]3CCCN3C)nc3c2CN(Cc2c(Cl)cccc2OC(F)F)C3)C[C@H]1C] | G | M + 1 = 577.3 | 0%/ 0% | 1H NMR (400 MHz, CD₃OD) δ 7.41-7.32 (m, 2H), 7.22-7.15 (m, 1H), 7.11- 6.74 (m, 2H), 6.24 (d, J = 16.5 Hz, 1H), 5.78 (d, J = 10.7 Hz, 1H), 4.50-4.31 (m, 2H), 4.31-4.26 (m, 2H), 4.26-4.15 (m, 3H), 4.14 (s, 2H), 3.99 (d, J = 19.4 Hz, 1H), 3.81 (s, 2H), 3.65-3.43 (m, 1H), 3.38 (d, J = 13.2 Hz, 1H), 3.20 (s, 1H), 3.11-3.05 (m, 1H), 2.79-2.71 (m, 1H), 2.49 (s, 3H), 2.36 (dd, J = 17.9, 9.0 Hz, 1H), 2.11- 2.03 (m, 1H), 1.81 (dq, J = 9.3, 5.2 Hz, 2H), 1.72- 1.63 (m, 1H), 1.25 (d, J = 13.9 Hz, 3H). |
| 1-106 | 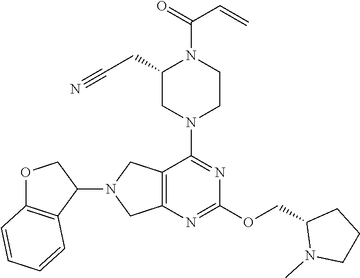 [SMILES: C=CC(=O)N1CCN(c2nc(OC[C@@H]3CCCN3C)nc3c2CN(C2COc4ccccc42)C3)C[C@@H]1CC#N] | A | M + 1 = 530.2 | 59%/ 75% | 1H NMR (400 MHz, CD₃OD) δ 7.43 (d, J = 7.1 Hz, 1H), 7.23 (dd, J = 11.4, 4.2 Hz, 1H), 7.04-6.69 (m, 3H), 6.27 (d, J = 16.6 Hz, 1H), 5.82 (d, J = 10.4 Hz, 1H), 4.96 (s, 1H), 4.79-4.56 (m, 4H), 4.56-4.36 (m, 2H), 4.31-4.13 (m, 2H), 4.07 (s, 1H), 3.87 (dd, J = 14.7, 5.3 Hz, 1H), 3.73 (d, J = 14.5 Hz, 1H), 3.55- 3.47 (m, 3H), 3.36 (s, 1H), 3.19-2.98 (m, 1H), 2.94-2.69 (m, 5H), 2.29 (dt, J = 15.1, 8.2 Hz, 1H), 2.18-1.81 (m, 3H). |
| 1-107 | 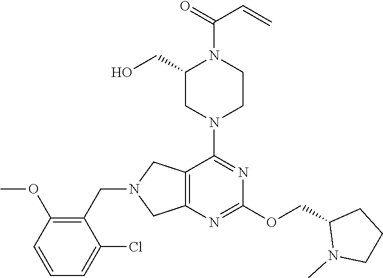 [SMILES: C=CC(=O)N1CCN(c2nc(OC[C@@H]3CCCN3C)nc3c2CN(Cc2c(Cl)cccc2OC)C3)C[C@@H]1CO] | B | M + 1 = 557.2 | 0%/ 3% | 1H NMR (400 MHz, CDCl₃) δ 7.20 (t, J = 8.2 Hz, 1H), 7.06-6.98 (m, 1H), 6.82 (d, J = 8.3 Hz, 1H), 6.59 (d, J = 11.2 Hz, 1H), 6.36 (d, J = 15.8 Hz, 1H), 5.76 (d, J = 10.6 Hz, 1H), 4.78 (d, J = 13.0 Hz, 1H), 4.48 (s, 1H), 4.32 (s, 1H), 4.11 (d, J = 8.4 Hz, 5H), 3.94-3.80 (m, 6H), 3.65- 3.48 (m, 2H), 3.23 (s, 2H), 3.11 (d, J = 14.6 Hz, 1H), 2.87 (s, 1H), 2.58 (s, 3H), 2.44 (s, 1H), 1.95 (dd, J = 64.6, 39.1 Hz, 6H). |
| 1-108 | 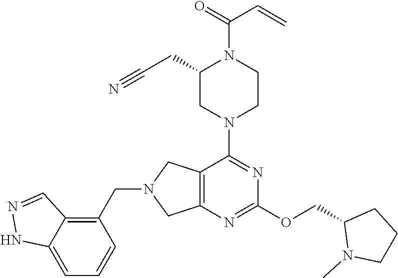 [SMILES: C=CC(=O)N1CCN(c2nc(OC[C@@H]3CCCN3C)nc3c2CN(Cc2cccc4[nH]ncc24)C3)C[C@@H]1CC#N] | A | M + 1 = 542.3 | 84%/ 97% | 1H NMR (400 MHz, CD₃OD) δ 8.42 (s, 1H), 8.28 (s, 1H), 7.50 (d, J = 8.4 Hz, 1H), 7.37 (dd, J = 8.3, 7.1 Hz, 1H), 7.18 (d, J = 6.9 Hz, 1H), 6.90-6.68 (m, 1H), 6.27 (d, J = 16.7 Hz, 1H), 5.81 (d, J = 9.6 Hz, 1H), 4.94 (s, 1H), 4.76 (t, J = 11.2 Hz, 3H), 4.49 (dd, J = 12.6, 7.4 Hz, 1H), 4.27 (s, 2H), 4.21 (s, 2H), 4.18- 3.89 (m, 2H), 3.87-3.76 (m, 3H), 3.71-3.63 (m, 1H), 3.49 (dd, J = 24.8, 13.8 Hz, 1H), 3.37 (d, J = 10.3 Hz, 1H), 3.21 (dd, J = 8.1, 3.2 Hz, 1H), 3.02 (s, 3H), 2.93-2.72 (m, 2H), 2.40-2.31 (m, 1H), 2.23- 2.05 (m, 2H), 1.99 (dd, J = 13.2, 6.0 Hz, 1H). |
| 1-109 | 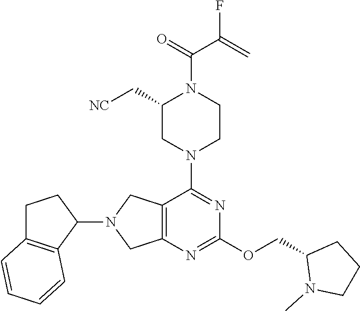 [SMILES: C=C(F)C(=O)N1CCN(c2nc(OC[C@@H]3CCCN3C)nc3c2CN(C2CCc4ccccc42)C3)C[C@@H]1CC#N] | A | M + 1 = 546.3 | 48%/ 68% | 1H NMR (400 MHz, CD₃OD) δ 7.42 (d, J = 6.9 Hz, 1H), 7.30-7.15 (m, 3H), 5.31 (dt, J = 14.6, 12.5 Hz, 2H), 4.95 (s, 1H), 4.68 (dd, J = 13.8, 5.6 Hz, 1H), 4.59-4.49 (m, 2H), 4.39 (dd, J = 11.9, 6.9 Hz, 1H), 4.29-3.87 (m, 4H), 3.83 (s, 2H), 3.66-3.32 (m, 4H), 3.13-3.04 (m, 1H), 3.02-2.69 (m, 7H), 2.35- 2.15 (m, 3H), 2.06-1.78 (m, 3H). |
| 1-110 | 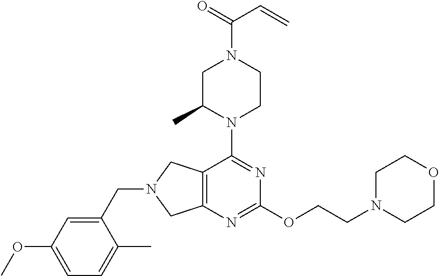 [SMILES: C=CC(=O)N1CCN(c2nc(OCCN3CCOCC3)nc3c2CN(Cc2cc(OC)ccc2C)C3)[C@@H](C)C1] | G | M + 1 = 537.2 | 0%/ 0% | 1H NMR (400 MHz, CD₃OD) δ 7.23 (d, 1H), 7.07 (s, 1H), 6.94-6.76 (m, 2H), 6.30 (d, 1H), 5.81 (d, 1H), 4.49 (br, 1H), 4.45- 4.44 (m, 3H), 4.21-4.04 (m, 4H), 3.87 (s, 2H), 3.77 (s, 3H), 3.70-3.68 (m, 6H), 3.61-3.54 (m, 1H), 3.33- 3.32 (d, 1H), 3.19-2.95 (m, 1H), 2.74-2.75 (m, 2H), 2.61-2.49 (m, 4H), 2.30 (s, 3H), 1.21 (d, 3H). |
| 1-111 | 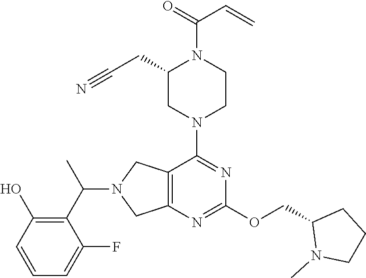 [SMILES: C=CC(=O)N1CCN(c2nc(OC[C@@H]3CCCN3C)nc3c2CN(C(C)c2c(O)cccc2F)C3)C[C@@H]1CC#N] | A | M + 1 = 550.3 | 87%/ 96% | 1H NMR (400 MHz, CD₃OD) 7.12 (d, J = 7.0 Hz, 1H), 6.75 (s, 1H), 6.58 (d, J = 7.1 Hz, 2H), 6.27 (d, J = 16.1 Hz, 1H), 5.82 (s, 1H), 4.95 (s, 1H), 4.65 (d, J = 61.0 Hz, 3H), 4.29 (ddd, J = 70.6, 39.3, 11.0 Hz, 6H), 3.81 (dd, J = 27.6, 14.2 Hz, 2H), 3.45 (d, J = 58.2 Hz, 3H), 3.20-3.02 (m, 1H), 2.84 (d, J = 47.2 Hz, 6H), 2.23 (s, 1H), 1.92 (d, J = 50.7 Hz, 3H), 1.53 (d, J = 5.5 Hz, 3H). |
| 1-112 | 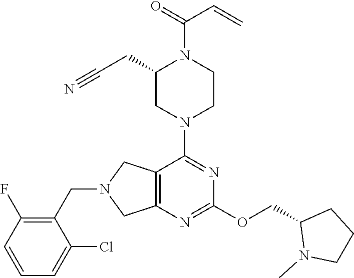 [SMILES: C=CC(=O)N1CCN(c2nc(OC[C@@H]3CCCN3C)nc3c2CN(Cc2c(F)cccc2Cl)C3)C[C@@H]1CC#N] | A | M + 1 = 554.2 | 98%/ 99% | 1H NMR (400 MHz, CD₃OD) δ 7.37-7.25 (m, 2H), 7.16-7.08 (m, 1H), 6.77 (s, 1H), 6.26 (d, J = 16.5 Hz, 1H), 5.81 (d, J = 10.1 Hz, 1H), 4.99 (s, 1H), 4.75- 4.44 (m, 2H), 4.31 (d, J = 5.8 Hz, 2H), 4.20 (s, 2H), 4.10 (d, J = 2.2 Hz, 3H), 3.78 (s, 2H), 3.50 (d, J = 27.1 Hz, 2H), 3.08 (dt, J = 9.6, 6.1 Hz, 2H), 2.91- 2.69 (m, 3H), 2.49 (s, 3H), 2.33 (dd, J = 18.7, 9.8 Hz, 1H), 2.10-2.00 (m, 1H), 1.80 (dq, J = 9.0, 5.0 Hz, 2H), 1.71-1.62 (m, 1H). |
| 1-113 | 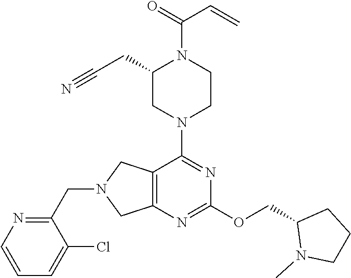 [SMILES: C=CC(=O)N1CCN(c2nc(OC[C@@H]3CCCN3C)nc3c2CN(Cc2ncccc2Cl)C3)C[C@@H]1CC#N] | A | M + 1 = 537.2 | 59%/ 89% | 1H NMR (400 MHz, CD₃OD) δ 8.49 (dd, J = 4.8, 1.2 Hz, 1H), 8.47 (s, 2H), 7.88 (dd, J = 8.1, 1.3 Hz, 1H), 7.35 (dd, J = 8.1, 4.8 Hz, 1H), 6.76 (s, 1H), 6.27 (d, J = 16.7 Hz, 1H), 5.81 (d, J = 10.1 Hz, 1H), 4.94 (d, J = 20.1 Hz, 1H), 4.72 (s, 2H), 4.45 (dd, J = 12.3, 7.2 Hz, 1H), 4.29 (s, 2H), 4.23 (s, 2H), 4.09 (s, 1H), 3.92 (s, 2H), 3.66 (s, 1H), 3.61- 3.44 (m, 2H), 3.35 (s, 2H), 3.15-3.02 (m, 2H), 2.94 (s, 3H), 2.79 (s, 2H), 2.36-2.26 (m, 1H), 2.13- 1.90 (m, 3H). |
| 1-114 | 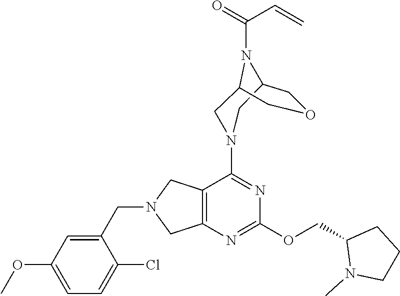 [SMILES: C=CC(=O)N1C2COCC1CN(c1nc(OC[C@@H]3CCCN3C)nc3c1CN(Cc1cc(OC)ccc1Cl)C3)C2] | A | M + 1 = 569.2 | 0%/ 0% | Intermediate. No NMR |
| 1-116 | 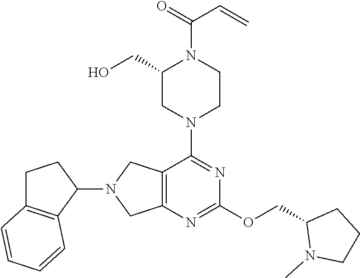 [SMILES: C=CC(=O)N1CCN(c2nc(OC[C@@H]3CCCN3C)nc3c2CN(C2CCc4ccccc42)C3)C[C@@H]1CO] | B | M + 1 = 519.2 | 2%/ 17% | 1H NMR (400 MHz, CDCl₃) δ 7.32 (d, J = 7.0 Hz, 1H), 7.28 (s, 1H), 7.18 (dd, J = 14.4, 7.1 Hz, 2H), 6.56 (d, J = 12.0 Hz, 1H), 6.32 (d, J = 16.2 Hz, 1H), 5.72 (d, J = 10.6 Hz, 1H), 4.74 (s, 1H), 4.52 (t, J = 6.3 Hz, 1H), 4.47-4.18 (m, 3H), 4.06 (d, J = 9.0 Hz, 3H), 3.91-3.79 (m, 3H), 3.61-3.47 (m, 2H), 3.21- 2.98 (m, 4H), 2.87-2.78 (m, 2H), 2.51 (s, 3H), 2.27- 2.17 (m, 2H), 2.14-2.00 (m, 4H), 1.81 (d, J = 42.3 Hz, 3H). |
| 1-117 | 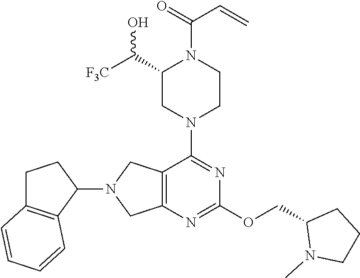 [SMILES: C=CC(=O)N1CCN(c2nc(OC[C@@H]3CCCN3C)nc3c2CN(C2CCc4ccccc42)C3)C[C@@H]1C(O)C(F)(F)F] | B | M + 1 = 587.2 | 0%/ 4% | 1H NMR (400 MHz, CD₃OD) δ 7.42 (d, J = 7.1 Hz, 1H), 7.32-7.11 (m, 3H), 6.74 (td, J = 16.4, 10.7 Hz, 1H), 6.21 (dd, J = 16.8, 7.7 Hz, 1H), 5.76 (dd, J = 18.4, 10.8 Hz, 1H), 5.05 (s, 1H), 4.69-4.38 (m, 5H), 4.34-3.98 (m, 4H), 3.96- 3.85 (m, 3H), 3.82 (ddd, J = 15.8, 7.9, 2.9 Hz, 1H), 3.67 (ddd, J = 11.8, 7.1, 5.1 Hz, 1H), 3.58 (t, J = 13.0 Hz, 1H), 3.47-3.34 (m, 1H), 3.30-3.16 (m, 2H), 3.16-3.01 (m, 4H), 2.96-2.82 (m, 1H), 2.42- 2.26 (m, 2H), 2.28-2.10 (m, 2H), 2.10-2.02 (m, 1H), 1.99 (ddd, J = 27.2, 13.4, 6.7 Hz, 1H). |
| 1-118 | 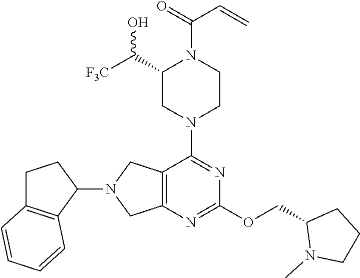 [SMILES: C=CC(=O)N1CCN(c2nc(OC[C@@H]3CCCN3C)nc3c2CN(C2CCc4ccccc42)C3)C[C@@H]1C(O)C(F)(F)F] | B | M + 1 = 587.2 | 0%/ 0% | Diastereoisomer of 1-117. 1H NMR (400 MHz,CD₃OD) δ 7.43 (d, J = 6.5 Hz, 1H), 7.34-7.14 (m, 3H), 6.74 (dd, J = 16.8, 10.2 Hz, 1H), 6.24 (t, J = 20.0 Hz, 1H), 5.79 (d, J = 10.4 Hz, 1H), 4.97 (s, 1H), 4.80-4.35 (m, 6H), 4.29 (s, 2H), 4.11 (d, J = 13.7 Hz, 1H), 3.87 (s, 2H), 3.78- 3.36 (m, 3H), 3.27- 2.93 (m, 6H), 2.90 (s, 1H), 2.23 (dd, J = 93.0. 48.8 Hz, 6H). |
| 1-119 | 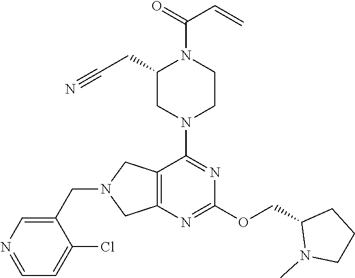 [SMILES: C=CC(=O)N1CCN(c2nc(OC[C@@H]3CCCN3C)nc3c2CN(Cc2cnccc2Cl)C3)C[C@@H]1CC#N] | A | M + 1 = 537.2 | 66%/ 94% | 1H NMR (400 MHz, CD₃OD) 8.42 (d, J = 5.4 Hz, 2H), 7.54 (d, J = 5.4 Hz, 1H), 6.76 (s, 1H), 6.27 (d, J = 16.5 Hz, 1H), 5.82 (d, J = 10.3 Hz, 1H), 4.95 (s, 1H), 4.74 (d, J = 11.6 Hz, 2H), 4.46 (dd, J = 12.5, 7.3 Hz, 1H), 4.18 (d, J = 40.4 Hz, 6H), 3.85 (s, 2H), 3.74 (s, 1H), 3.60 (dt, J = 29.6, 13.8 Hz, 2H), 3.37 (s, 2H), 3.18-3.10 (m, 1H), 2.98 (s, 3H), 2.84 (d, J = 47.1 Hz, 2H), 2.33 (dt, J = 14.9, 7.3 Hz, 1H), 2.21-1.91 (m, 3H). |
| 1-120 | 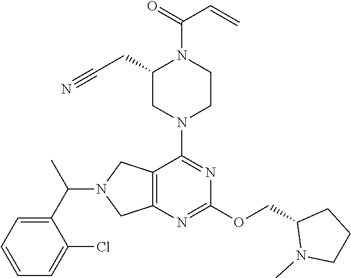 [SMILES: C=CC(=O)N1CCN(c2nc(OC[C@@H]3CCCN3C)nc3c2CN(C(C)c2ccccc2Cl)C3)C[C@@H]1CC#N] | A | M + 1 = 550.2 | 57%/ 74% | 1H NMR (400 MHz, Methanol-d4) δ 8.46 (s, 2H), 7.63 (d, J = 7.7 Hz, 1H), 7.43-7.22 (m, 3H), 6.75 (s, 1H), 6.27 (d, J = 16.7 Hz, 1H), 5.81 (d, J = 9.7 Hz, 1H), 4.93 (s, 1H), 4.74 (d, J = 12.0 Hz, 2H), 4.49 (dd, J = 12.5, 7.4 Hz, 1H), 4.39- 4.31 (m, 1H), 4.25- 3.88 (m, 4H), 3.74 (dd, J = 19.2, 5.9 Hz, 3H), 3.67- 3.60 (m, 1H), 3.55-3.33 (m, 2H), 3.26-3.04 (m, 2H), 2.99 (s, 3H), 2.95- 2.71 (m, 2H), 2.34 (dq, J = 14.8, 8.0 Hz, 1H), 2.19- 1.92 (m, 3H), 1.44 (d, J = 6.5 Hz, 3H). |
| 1-121 | 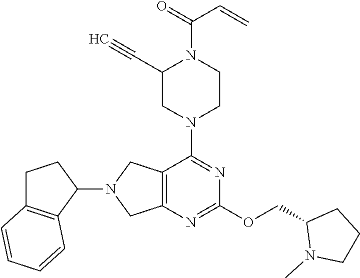 [SMILES: C#CC1CN(c2nc(OC[C@@H]3CCCN3C)nc3c2CN(C2CCc4ccccc42)C3)CCN1C(=O)C=C] | B | M + 1 = 513.3 | 0%/ 4% | 1H NMR (400 MHz, CD₃OD) δ 8.31 (s, 2H), 7.47- 7.40 (m, 1H), 7.30-7.19 (m, 3H), 6.77 (dd, J = 16.7, 10.6 Hz, 1H), 6.29 (dd, J = 16.8, 1.6 Hz, 1H), 5.83 (dd, J = 10.6, 1.5 Hz, 1H), 5.54 (s, 1H), 5.19 (s, 1H), 4.65 (ddd, J = 18.0, 12.3, 4.3 Hz, 3H), 4.50 (dd, J = 12.6, 7.2 Hz, 1H), 4.35 (dd, J = 23.6, 12.0 Hz, 2H), 4.28-4.20 (m, 1H), 4.08 (d, J = 15.0 Hz, 1H), 3.89 (s, 2H), 3.83 (dt, J = 7.6, 4.8 Hz, 1H), 3.69 (dd, J = 12.0, 5.6 Hz, 1H), 3.58 (dd, J = 21.6, 8.3 Hz, 1H), 3.22 (d, J = 11.3 Hz, 1H), 3.15-3.05 (m, 2H), 3.03 (d, J = 2.1 Hz, 3H), 2.92- 2.85 (m, 1H), 2.73 (s, 1H), 2.34 (ddd, J = 22.0, 13.2, 6.7 Hz, 2H), 2.19 (ddd, J = 15.2, 10.2, 4.4 Hz, 2H), 2.12-1.97 (m, 2H) |
| 1-122 | 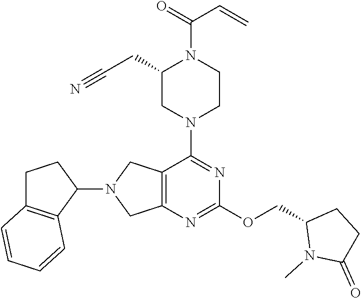 [SMILES: C=CC(=O)N1CCN(c2nc(OC[C@@H]3CCC(=O)N3C)nc3c2CN(C2CCc4ccccc42)C3)C[C@@H]1CC#N] | B | M + 1 = 542.3 | 21%/ 57% | 1H NMR (400 MHz, CD₃OD) δ 7.41 (d, J = 7.0 Hz, 1H), 7.22 (dt, J = 14.2, 6.8 Hz, 3H), 6.78 (d, J = 9.7 Hz, 1H), 6.27 (d, J = 16.6 Hz, 1H), 5.81 (d, J = 9.9 Hz, 1H), 4.97 (s, 1H), 4.66 (t, J = 24.1 Hz, 2H), 4.51 (d, J = 5.9 Hz, 1H), 4.36 (dd, J = 11.6, 4.0 Hz, 1H), 4.22 (s, 3H), 4.11-3.91 (m, 2H), 3.82 (s, 2H), 3.52 (s, 1H), 3.39 (s, 1H), 3.20- 3.00 (m, 2H), 2.86 (s, 6H), 2.60-2.48 (m, 1H), 2.39-2.15 (m, 4H), 1.99 (dd, J = 13.7, 8.6 Hz, 1H). |
| 1-123 | 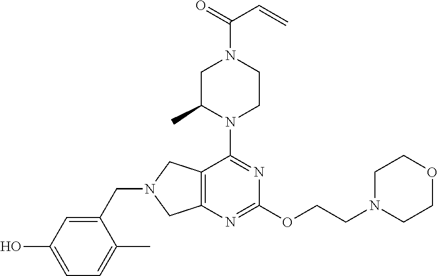 [SMILES: C=CC(=O)N1CCN(c2nc(OCCN3CCOCC3)nc3c2CN(Cc2cc(O)ccc2C)C3)[C@@H](C)C1] | B | M + 1 = 523.3 | 0%/ 2% | 1H NMR (400 MHz, DMSO-d6) δ 9.2 (d, 1H), 6.96-6.90 (m, 1H), 6.62-6.60 (m, 2H), 6.59-6.57 (m, 1H), 6.20-6.18 (m, 1H), 5.73- 5.72 (m, 1H), 5.35-5.15 (m, 1H), 5.14-4.93 (m, 3H), 4.85-4.59 (m, 2H), 4.06 (s, 3H), 4.06-4.03 (m, 1H), 3.62-3.37 (m, 4H), 2.99- 2.83 (m, 3H), 2.67-2.64 (m, 4H), 2.51 (s, 3H), 2.42 (d, 3H), 0.96-0.94 (m, 3H). |
| 1-124 | 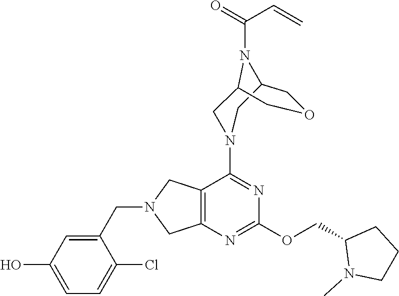 [SMILES: C=CC(=O)N1C2COCC1CN(c1nc(OC[C@@H]3CCCN3C)nc3c1CN(Cc1cc(O)ccc1Cl)C3)C2] | A | M + 1 = 555.3 | 21%/ 67% | 1H NMR (400 MHz, CD₃OD) δ 8.53 (s, 1H), 7.19 (d, J = 8.6 Hz, 1H), 6.99 (d, J = 3.0 Hz, 1H), 6.78 (dd, J = 16.7, 10.6 Hz, 1H), 6.70 (dd, J = 8.7, 2.9 Hz, 1H), 6.33 (dd, J = 16.7, 1.9 Hz, 1H), 5.84 (dd, J = 10.6, 1.9 Hz, 1H), 4.60 (br, 3H), 4.51 (ddd, J = 11.8, 4.4, 1.8 Hz, 1H), 4.41 (dd, J = 11.8, 6.8 Hz, 1H), 4.26 (s, 1H), 4.21 (s, 2H), 4.03 (t, J = 10.3 Hz, 2H), 3.99 (s, 2H), 3.82-3.69 (m, 4H), 3.51-3.35 (m, 3H), 2.86- 2.70 (m, 5H), 2.24 (dq, J = 12.6, 8.0 Hz, 1H), 2.05- 1.78 (m, 3H). |
| 1-125 | 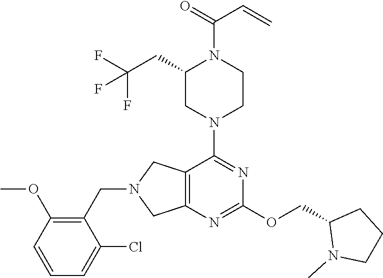 [SMILES: C=CC(=O)N1CCN(c2nc(OC[C@@H]3CCCN3C)nc3c2CN(Cc2c(Cl)cccc2OC)C3)C[C@@H]1CC(F)(F)F] | B | M + 1 = 609.3 | 0%/ 0% | 1H NMR (400 MHz, CD₃OD) δ 7.28 (t, J = 8.2 Hz, 1H), 7.01 (dd, J = 19.6, 8.2 Hz, 2H), 6.85-6.70 (m, 1H), 6.25 (t, J = 15.1 Hz, 1H), 5.80 (d, J = 9.9 Hz, 1H), 5.15 (s, 1H), 4.58 (t, J = 53.7 Hz, 2H), 4.34- 4.23 (m, 2H), 4.21-4.07 (m, 5H), 3.88 (s, 3H), 3.80 (s, 2H), 3.60-3.46 (m, 1H), 3.25 (s, 2H), 3.08 (dt, J = 9.4, 8.0 Hz, 2H), 2.78-2.68 (m, 1H), 2.57 (d, J = 10.5 Hz, 1H), 2.48 (s, 3H), 2.35 (dd, J = 17.9, 9.1 Hz, 1H), 2.11-2.01 (m, 1H), 1.81 (td, J = 12.8, 7.9 Hz, 2H), 1.66 (dt, J = 20.0, 7.4 Hz, 1H). |
| 1-126 | 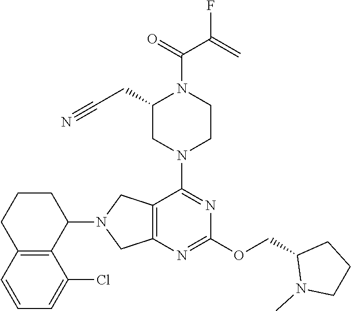 [SMILES: C=C(F)C(=O)N1CCN(c2nc(OC[C@@H]3CCCN3C)nc3c2CN(C2CCCc4cccc(Cl)c42)C3)C[C@@H]1CC#N] | H | M + 1 = 594.3 | 53%/ 88% | 1H NMR (400 MHz, CD₃OD) δ 7.25 (d, J = 7.7 Hz, 1H), 7.19 (t, J = 7.7 Hz, 1H), 7.11 (d, J = 7.4 Hz, 1H), 5.31 (d, J = 20.6 Hz, 2H), 4.87 (s, 1H), 4.64 (d, J = 13.9 Hz, 1H), 4.49-4.23 (m, 5H), 4.19 (d, J = 11.6 Hz, 1H), 4.04 (d, J = 11.1 Hz, 1H), 3.97-3.85 (m, 1H), 3.66-3.43 (m, 2H), 3.34 (s, 1H), 3.25 (s, 1H), 3.15 (s, 1H), 3.03 (ddd, J = 16.4, 7.8, 5.0 Hz, 1H), 2.96-2.84 (m, 3H), 2.77 (s, 1H), 2.56 (s, 3H), 2.47 (d, J = 8.7 Hz, 1H), 2.33-2.23 (m, 1H), 2.17- 2.05 (m, 2H), 1.90-1.80 (m, 2H), 1.73 (d, J = 6.3 Hz, 2H), 1.63-1.50 (m, 1H). |
| 1-127 | 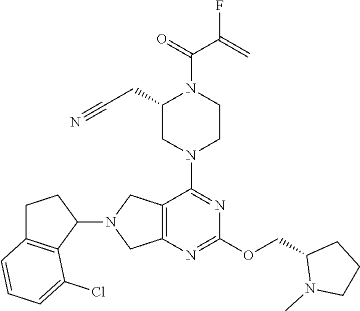 [SMILES: C=C(F)C(=O)N1CCN(c2nc(OC[C@@H]3CCCN3C)nc3c2CN(C2CCc4cccc(Cl)c42)C3)C[C@@H]1CC#N] | A | M + 1 = 580.2 | 64%/ 95% | 1H NMR (400 MHz, CD₃OD) δ 8.53 (s, 1H), 7.22 (t, J = 6.9 Hz, 3H), 5.31 (dd, J = 33.2, 12.7 Hz, 2H), 4.65 (dd, J = 28.3, 9.2 Hz, 2H), 4.51 (d, J = 10.9 Hz, 1H), 4.38 (dd, 11.6, 6.9 Hz, 1H), 4.23 (d, J = 10.7 Hz, 3H), 4.11-3.93 (m, 1H), 3.81 (t, J = 14.6 Hz, 2H), 3.47 (s, 1H), 3.34 (s, 2H), 3.27-3.09 (m, 3H), 2.90 (t, J = 12.0 Hz, 4H), 2.75 (d, J = 19.1 Hz, 4H), 2.42-2.29 (m, 1H), 2.26- 2.11 (m, 2H), 1.94 (d, J = 1.1 Hz, 2H), 1.83 (dd, J = 13.1, 7.3 Hz, 1H). |
| 1-128 | 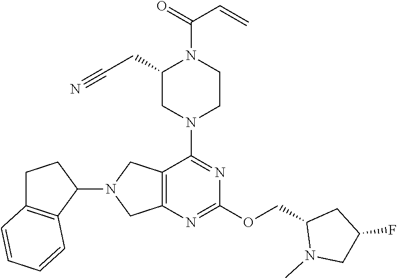 [SMILES: C=CC(=O)N1CCN(c2nc(OC[C@@H]3C[C@H](F)CN3C)nc3c2CN(C2CCc4ccccc42)C3)C[C@@H]1CC#N] | B | M + 1 = 546.2 | 52%/ 63% | 1H NMR (400 MHz, CD₃OD) δ 8.33 (s, 3H), 7.44 (d, J = 7.1 Hz, 1H), 7.30-7.19 (m, 3H), 6.78 (s, 1H), 6.28 (d, J = 16.6 Hz, 1H), 5.83 (d, J = 10.4 Hz, 1H), 5.39 (d, J = 4.4 Hz, 0.5H), 5.29-5.21 (m, 0.5H), 4.61 (ddd, J = 15.9, 11.5, 9.1 Hz, 4H), 4.46 (dd, J = 11.8, 8.2 Hz, 1H), 4.27 (d, J = 11.8 Hz, 2H), 4.11 (d, J = 40.2 Hz, 2H), 3.88 (s, 2H), 3.72-3.62 (m, 1H), 3.51 (d, J = 19.0 Hz, 2H), 3.37 (d, J = 10.9 Hz, 1H), 3.10 (dt, J = 15.1, 7.5 Hz, 2H), 2.95-2.80 (m, 6H), 2.79-2.58 (m, 2H), 2.22 (dddd, J = 27.5, 18.5, 12.9, 6.3 Hz, 3H). |
| 1-129 | 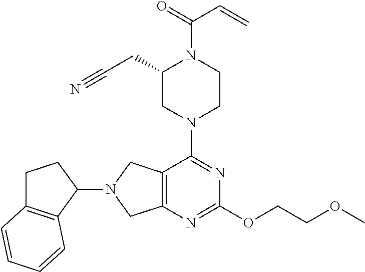 [SMILES: C=CC(=O)N1CCN(c2nc(OCCOC)nc3c2CN(C2CCc4ccccc42)C3)C[C@@H]1CC#N] | B | M + 1 = 489.2 | 26%/ 64% | 1H NMR (400 MHz, CD₃OD) δ 7.39 (d, J = 7.3 Hz, 1H), 7.18 (dq, J = 14.2, 7.0 Hz, 3H), 6.71 (s, 1H), 6.20 (d, J = 17.1 Hz, 1H), 5.75 (d, J = 10.7 Hz, 1H), 4.88 (d, J = 29.8 Hz, 1H), 4.51 (d, J = 28.4 Hz, 3H), 4.42-4.33 (m, 2H), 4.24 (d, J = 11.7 Hz, 2H), 4.11 (d, J = 11.3 Hz, 1H), 3.98 (s, 1H), 3.85 (s, 2H), 3.64 (t, J = 4.7 Hz, 2H), 3.54-3.28 (m, 5H), 3.04 (dt, J = 15.6, 7.9 Hz, 2H), 2.86-2.78 (m, 2H), 2.30-2.15 (m, 2H). |
| 1-130 | 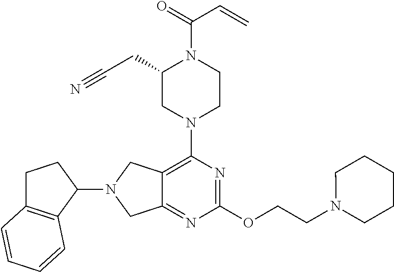 [SMILES: C=CC(=O)N1CCN(c2nc(OCCN3CCCCC3)nc3c2CN(C2CCc4ccccc42)C3)C[C@@H]1CC#N] | A | M + 1 = 544.3 | 51%/ 86% | 1H NMR (400 MHz, CD₃OD) δ 8.40 (s, 2H), 7.43 (d, J = 7.0 Hz, 1H), 7.23 (dt, J = 14.6, 6.8 Hz, 3H), 6.78 (s, 1H), 6.27 (d, J = 17.0 Hz, 1H), 5.82 (d, J = 9.2 Hz, 1H), 4.95 (s, 1H), 4.74 (s, 1H), 4.66-4.58 (m, 1H), 4.53 (d, J = 4.6 Hz, 3H), 4.25 (s, 2H), 4.18 (s, 1H), 4.05 (s, 1H), 3.85 (s, 2H), 3.75-3.71 (m, 3H), 3.54 (s, 1H), 3.35 (s, 1H), 3.08 (d, J = 8.0 Hz, 2H), 2.94 (s, 2H), 2.87 (dd, J = 18.5, 12.0 Hz, 3H), 2.75 (s, 4H), 2.35-2.17 (m, 2H). |
| 1-131 | 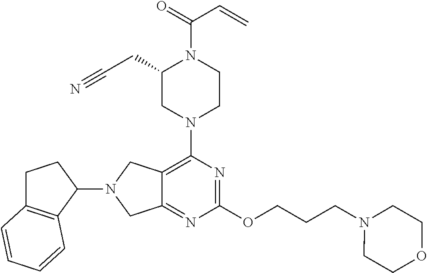 [SMILES: C=CC(=O)N1CCN(c2nc(OCCCN3CCOCC3)nc3c2CN(C2CCc4ccccc42)C3)C[C@@H]1CC#N] | A | M + 1 = 558.3 | 76%/ 97% | 1H NMR (400 MHz, CD₃OD) δ 7.43 (d, 1H), 7.41- 7.21 (m, 3H), 6.78 (br, 1H), 6.29 (d, 1H), 5.83 (d, 1H), 4.88-4.86 (m, 2H), 4.57-4.51 (m, 2H), 4.38-4.37 (m, 2H), 4.22-4.05 (m, 4H), 3.70 (s, 2H), 3.49 (s, 4H), 3.51-3.50 (m, 1H), 3.13-3.09 (m, 2H), 3.05-2.88 (m, 3H), 2.60-2.52 (m, 6H), 2.51-2.49 (m, 2H), 1.99- 1.96 (m, 2H). |
| 1-132 | 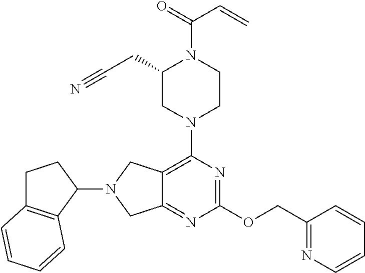 [SMILES: C=CC(=O)N1CCN(c2nc(OCc3ccccn3)nc3c2CN(C2CCc4ccccc42)C3)C[C@@H]1CC#N] | B | M + 1 = 522.3 | 24%/ 68% | 1H NMR (400 MHz, DMSO) δ 8.54 (d, J = 4.2 Hz, 1H), 7.79 (td, J = 7.7, 1.7 Hz, 1H), 7.41-7.16 (m, 6H), 6.89-6.75 (m, 1H), 6.16 (dd, J = 16.6, 1.5 Hz, 1H), 5.75 (d, J = 10.4 Hz, 1H), 5.33 (d, J = 14.4 Hz, 2H), 4.78 (d, J = 68.0 Hz, 1H), 4.45 (dd, J = 11.2, 5.6 Hz, 1H), 4.33-3.96 (m, 5H), 3.76-3.66 (m, 2H), 3.53-3.36 (m, 1H), 3.30- 3.08 (m, 2H), 3.01-2.92 (m, 2H), 2.83-2.74 (m, 2H), 2.14 (ddd, J = 22.1, 13.8, 7.4 Hz, 2H). |
| 1-133 | 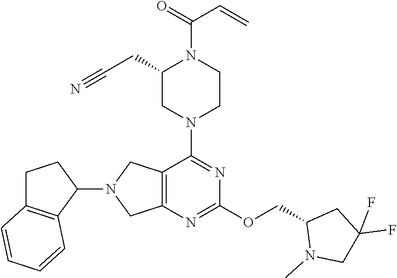 [SMILES: C=CC(=O)N1CCN(c2nc(OC[C@@H]3CC(F)(F)CN3C)nc3c2CN(C2CCc4ccccc42)C3)C[C@@H]1CC#N] | B | M + 1 = 564.3 | 84%/ 90% | 1H NMR (400 MHz, CD₃OD) δ 8.43 (s, 0H), 7.42 (d, J = 7.2 Hz, 1H), 7.22 (dq, J = 14.0, 7.1 Hz, 3H), 6.77 (s, 1H), 6.27 (d, J = 16.7 Hz, 1H), 5.82 (d, J = 10.0 Hz, 1H), 4.98 (s, 1H), 4.59 (s, 1H), 4.53 (dd, J = 12.0, 5.3 Hz, 1H), 4.39 (d, J = 4.9 Hz, 2H), 4.32- 3.99 (m, 4H), 3.83 (s, 2H), 3.50 (d, J = 22.5 Hz, 1H), 3.41-3.33 (m, 2H), 3.05 (ddd, J = 33.6, 14.9, 6.9 Hz, 3H), 2.87 (dd, J = 18.1, 11.8 Hz, 3H), 2.71 (ddd, J = 18.1, 15.4, 11.4 Hz, 1H), 2.55-2.42 (m, 4H), 2.33-2.15 (m, 3H). |
| 1-134 | 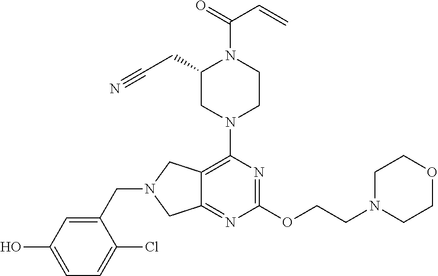 [SMILES: C=CC(=O)N1CCN(c2nc(OCCN3CCOCC3)nc3c2CN(Cc2cc(O)ccc2Cl)C3)C[C@@H]1CC#N] | A | M + 1 = 568.2 | 98%/ 98% | 1H NMR (400 MHz, CD₃OD) δ 7.19 (d, J = 8.7 Hz, 1H), 6.97 (d, J = 2.8 Hz, 1H), 6.77 (s, 1H), 6.69 (dd, J = 8.6, 2.8 Hz, 1H), 6.27 (d, J = 16.7 Hz, 1H), 5.81 (d, J = 10.1 Hz, 1H), 4.99 (s, 1H), 4.62 (d, J = 13.9 Hz, 1H), 4.48 (t, J = 5.3 Hz, 2H), 4.11 (d, J = 52.7 Hz, 4H), 3.97 (s, 2H), 3.78 (s, 2H), 3.71-3.64 (m, 4H), 3.54 (s, 1H), 3.39 (s, 1H), 3.13 (s, 1H), 2.93- 2.73 (m, 4H), 2.59 (s, 4H). |
| 1-135 | 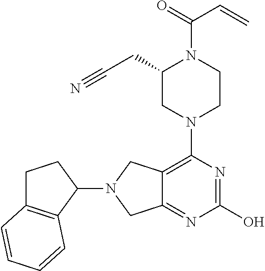 [SMILES: C=CC(=O)N1CCN(c2nc(O)nc3c2CN(C2CCc4ccccc42)C3)C[C@@H]1CC#N] | B | M + 1 = 431.2 | 0%/ 7% | 1H NMR (400 MHz, CD₃OD) δ 8.19 (s, 0.5H), 7.40 (d, J = 7.0 Hz, 1H), 7.30- 7.13 (m, 3H), 6.80 (dd, J = 29.3, 16.9 Hz, 1H), 6.27 (d, J = 16.6 Hz, 1H), 5.82 (d, J = 10.5 Hz, 1H), 5.05- 4.89 (m, 1H), 4.77 (dd, J = 30.3, 16.1 Hz, 1H), 4.54 (dt, J = 22.2, 9.7 Hz, 2H), 4.37-3.99 (m, 4H), 3.84 (d, J = 2.9 Hz, 2H), 3.62- 3.47 (m, 1H), 3.19-3.00 (m, 2H), 3.00-2.75 (m, 3H), 2.30 (td, J = 13.7, 7.4 Hz, 1H), 2.21-2.12 (m, 1H). |
| 1-136 | 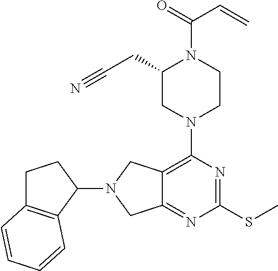 [SMILES: C=CC(=O)N1CCN(c2nc(SC)nc3c2CN(C2CCc4ccccc42)C3)C[C@@H]1CC#N] | B | M + 1 = 461.2 | 16%/ 56% | 1H NMR (400 MHz, CD₃OD) δ 7.42 (d, J = 7.0 Hz, 1H), 7.29-7.16 (m, 3H), 6.89-6.73 (m, 1H), 6.27 (d, J = 16.7 Hz, 1H), 5.82 (d, J = 10.4 Hz, 1H), 5.00 (s, 1H), 4.75 (s, 1H), 4.61- 4.48 (m, 2H), 4.22 (d, J = 14.1 Hz, 3H), 4.07 (d, J = 11.6 Hz, 1H), 3.83 (d, J = 2.1 Hz, 2H), 3.55 (s, 1H), 3.40 (d, J = 13.2 Hz, 1H), 3.17-2.99 (m, 2H), 2.87 (dd, J = 13.7, 7.6 Hz, 2H), 2.51 (s, 3H), 2.32-2.18 (m, 2H). |
| 1-138 | 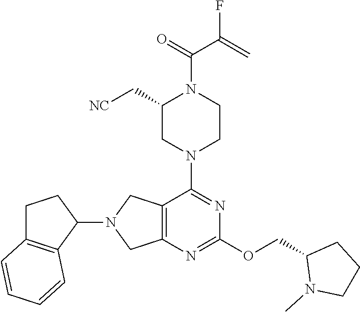 [SMILES: C=C(F)C(=O)N1CCN(c2nc(OC[C@@H]3CCCN3C)nc3c2CN(C2CCc4ccccc42)C3)C[C@@H]1CC#N] | A | M + 1 = 546.3 | 38%/ 54% | 1H NMR (400 MHz, CD₃OD) δ 7.42 (d, J = 7.0 Hz, 1H), 7.22 (dq, J = 14.2, 7.1 Hz, 3H), 5.40-5.21 (m, 2H), 4.61 (d, J = 14.0 Hz, 1H), 4.51 (t, J = 5.7 Hz, 1H), 4.31 (d, J = 5.7 Hz, 2H), 4.23 (q, J = 11.3 Hz, 3H), 3.82 (s, 2H), 3.37 (d, J = 13.2 Hz, 1H), 3.25 (s, 1H), 3.12-3.03 (m, 2H), 2.95-2.80 (m, 3H), 2.76- 2.66 (m, 1H), 2.48 (s, 3H), 2.39-2.00 (m, 6H), 1.79 (dd, J = 13.2, 7.5 Hz, 2H), 1.72-1.49 (m, 2H). |
| 1-139 | 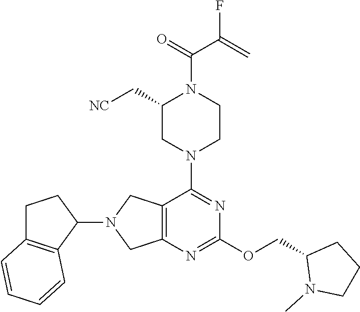 [SMILES: C=C(F)C(=O)N1CCN(c2nc(OC[C@@H]3CCCN3C)nc3c2CN(C2CCc4ccccc42)C3)C[C@@H]1CC#N] | A | M + 1 = 546.3 | 0%/ 5% | 1H NMR (400 MHz, CD₃OD) δ 7.42 (d, J = 7.0 Hz, 1H), 7.28-7.16 (m, 3H), 5.37-5.23 (m, 2H), 4.59 (d, J = 14.0 Hz, 1H), 4.52 (dd, J = 7.0, 5.1 Hz, 1H), 4.31 (d, J = 6.3 Hz, 2H), 4.22 (dd, J = 8.4, 6.6 Hz, 3H), 3.82 (s, 2H), 3.37 (d, J = 12.1 Hz, 1H), 3.25 (s, 1H), 3.06 (dt, J = 9.5, 6.9 Hz, 2H), 2.95-2.81 (m, 3H), 2.75-2.66 (m, 1H), 2.47 (s, 3H), 2.38-2.05 (m, 6H), 1.83-1.75 (m, 2H), 1.72-1.59 (m, 2H). |
| 1-140 | 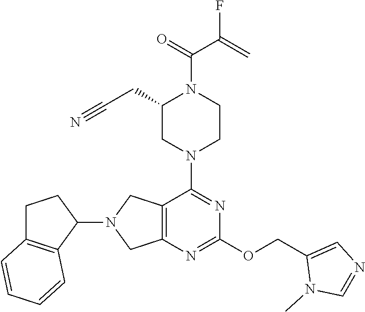 [SMILES: C=C(F)C(=O)N1CCN(c2nc(OCc3cncn3C)nc3c2CN(C2CCc4ccccc42)C3)C[C@@H]1CC#N] | B | M + 1 = 543.3 | 0%/ 0% | 1H NMR (400 MHz, CD₃OD) δ 8.25 (s, 1H), 7.87 (s, 1H), 7.44 (d, J = 7.2 Hz, 1H), 7.23 (ddd, J = 21.7, 14.9, 9.9 Hz, 4H), 5.45- 5.27 (m, 4H), 4.69 (dd, J = 13.4, 4.0 Hz, 1H), 4.59- 4.55 (m, 1H), 4.35-3.91 (m, 5H), 3.87 (s, 2H), 3.78 (s, 3H), 3.47 (dd, J = 53.2, 31.4 Hz, 2H), 3.15- 2.81 (m, 5H), 2.35-2.21 (m, 2H). |
| 1-141 | 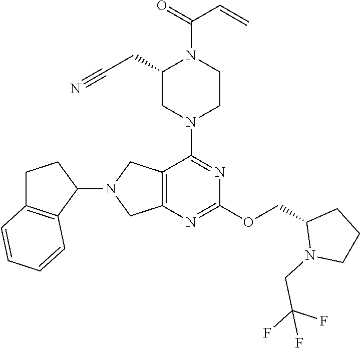 [SMILES: C=CC(=O)N1CCN(c2nc(OC[C@@H]3CCCN3CC(F)(F)F)nc3c2CN(C2CCc4ccccc42)C3)C[C@@H]1CC#N] | B | M + 1 = 596.3 | 51%/ 71% | 1H NMR (400 MHz, CD₃OD) δ 7.42 (d, J = 7.0 Hz, 1H), 7.27-7.17 (m, 3H), 6.78 (d, J = 12.8 Hz, 1H), 6.27 (d, J = 16.6 Hz, 1H), 5.81 (d, J = 10.5 Hz, 1H), 4.98 (s, 1H), 4.53 (dt, J = 11.8, 6.2 Hz, 2H), 4.30- 3.99 (m, 6H), 3.82 (d, J = 2.0 Hz, 2H), 3.65 (dq, J = 14.9, 10.5 Hz, 1H), 3.58- 3.32 (m, 2H), 3.25 (dd, J = 11.5, 7.8 Hz, 1H), 3.19- 3.02 (m, 4H), 2.96-2.76 (m, 3H), 2.56 (dd, J = 16.7, 8.5 Hz, 1H), 2.34-2.14 (m, 2H), 2.05-1.93 (m, 1H), 1.90-1.77 (m, 2H), 1.67 (ddd, J = 13.2, 8.0, 5.6 Hz, 1H). |
| 1-142 | 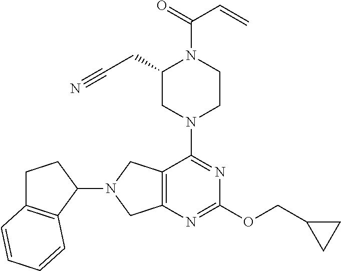 [SMILES: C=CC(=O)N1CCN(c2nc(OCC3CC3)nc3c2CN(C2CCc4ccccc42)C3)C[C@@H]1CC#N] | B | M + 1 = 485.3 | 7%/ 36% | 1H NMR (400 MHz, CD₃OD) δ 7.42 (d, J = 7.1 Hz, 1H), 7.29-7.16 (m, 3H), 6.79 (d, J = 8.6 Hz, 1H), 6.27 (d, J = 16.6 Hz, 1H), 5.82 (d, J = 10.2 Hz, 1H), 4.98 (s, 1H), 4.63 (d, J = 86.2 Hz, 3H), 4.34-3.97 (m, 6H), 3.81 (s, 2H), 3.61- 3.32 (m, 2H), 3.25- 3.04 (m, 2H), 2.85 (dd, J = 15.2, 7.6 Hz, 3H), 2.33- 2.15 (m, 2H), 0.63-0.54 (m, 2H), 0.33 (q, J = 4.8 Hz, 2H). |
| 1-143 | 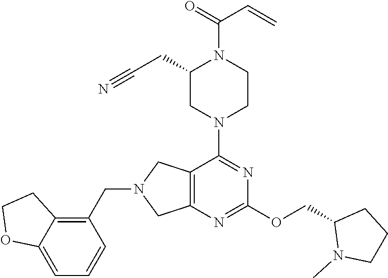 [SMILES: C=CC(=O)N1CCN(c2nc(OC[C@@H]3CCCN3C)nc3c2CN(Cc2cccc4c2CCO4)C3)C[C@@H]1CC#N] | A | M + 1 = 544.3 | 48%/ 70% | 1H NMR (400 MHz, CD₃OD) δ 7.08 (t, 1H), 6.83 (m, 2H), 6.66 (d, 1H), 6.27 (d, 1H), 5.82 (d, 1H), 4.93 (s, 1H), 4.76 (d, 2H), 4.53 (d,, 3H), 4.13-4.10 (m, 4H), 3.89 (s, 2H), 3.75 (s, 3H), 3.65-3.50 (m, 2H), 3.36-3.30 (m, 1H), 3.25- 3.23 (m, 3H), 3.13-3.10 (m, 1H), 2.98 (s, 3H), 2.78- 2.76 (m, 2H), 2.33-2.30 (m, 1H), 2.20-1.93 (m, 3H). |
| 1-144 | 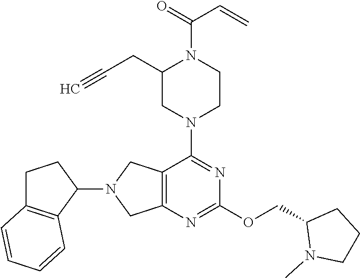 [SMILES: C#CCC1CN(c2nc(OC[C@@H]3CCCN3C)nc3c2CN(C2CCc4ccccc42)C3)CCN1C(=O)C=C] | J | M + 1 = 527.3 | 15%/ 35% | 1H NMR (400 MHz, CD₃OD) δ 8.50 (s, 1H), 7.42 (d, J = 6.6 Hz, 1H), 7.24 (dt, J = 14.3, 6.9 Hz, 3H), 6.93- 6.67 (m, 1H), 6.24 (d, J = 16.7 Hz, 1H), 5.79 (d, J = 10.5 Hz, 1H), 5.43-5.09 (m, 1H), 4.80-4.69 (m, 1H), 4.46 (dddd, J = 59.4, 46.8, 20.9, 8.6 Hz, 8H), 4.03 (s, 1H), 3.87 (s, 2H), 3.70-3.47 (m, 3H), 3.08 (dt, J = 24.2, 8.5 Hz, 3H), 2.97-2.83 (m, 4H), 2.59 (s, 1H), 2.30 (d, J = 7.7 Hz, 2H), 2.24-1.88 (m, 5H). |
| 1-145 | 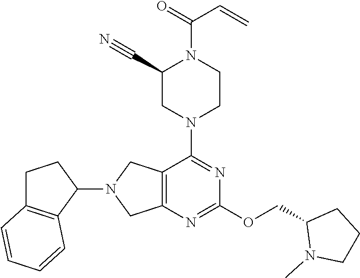 [SMILES: C=CC(=O)N1CCN(c2nc(OC[C@@H]3CCCN3C)nc3c2CN(C2CCc4ccccc42)C3)C[C@H]1C#N] | A | M + 1 = 514.2 | 60%/ 74% | 1H NMR (400 MHz, CD₃OD) δ 7.44 (d, J = 7.0 Hz, 1H), 7.23 (dq, J = 14.2, 7.0 Hz, 3H), 6.79 (dd, J = 16.7, 10.6 Hz, 1H), 6.35 (d, J = 16.6 Hz, 1H), 5.89 (d, J = 10.6 Hz, 1H), 5.70 (s, 1H), 4.75 (t, J = 13.0 Hz, 1H), 4.61-4.50 (m, 1H), 4.47-4.12 (m, 6H), 3.85 (s, 2H), 3.83 (d, J = 16.6 Hz, 2H), 3.54-3.33 (m, 2H), 3.24 (t, J = 8.2 Hz, 1H), 3.14-3.02 (m, 2H), 2.95-2.74 (m, 2H), 2.53 (s, 3H), 2.41 (s, 1H), 2.31 (dt, J = 21.7, 7.0 Hz, 1H), 2.28-2.15 (m, 1H), 2.10 (dt, J = 16.9, 8.3 Hz, 1H), 1.91-1.77 (m, 2H), 1.72 (dd, J = 12.6, 6.8 Hz, 1H). |
| 1-146 | 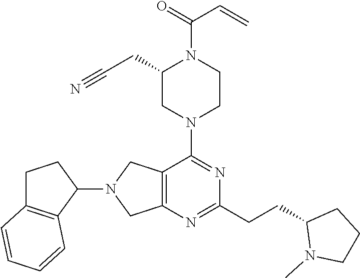 [SMILES: C=CC(=O)N1CCN(c2nc(CC[C@@H]3CCCN3C)nc3c2CN(C2CCc4ccccc42)C3)C[C@@H]1CC#N] | B | M + 1 = 526.3 | 7%/ 33% | 1H NMR (400 MHz, CD₃OD) δ 8.45 (s, 5H), 7.43 (d, J = 7.1 Hz, 1H), 7.25 (ddd, J = 23.4, 12.6, 7.2 Hz, 3H), 6.78 (s, 1H), 6.28 (d, J = 16.8 Hz, 1H), 5.82 (d, J = 10.0 Hz, 1H), 4.98 (s, 1H), 4.77 (s, 2H), 4.55 (d, J = 7.0 Hz, 1H), 4.27 (d, J = 12.8 Hz, 2H), 4.12 (s, 2H), 3.90 (s, 2H), 3.62 (s, 1H), 3.48 (dd, J = 3.3, 1.6 Hz, 1H), 3.36 (dd, J = 3.3, 1.7 Hz, 2H), 3.16-3.07 (m, 2H), 2.96-2.73 (m, 8H), 2.46 (s, 1H), 2.38- 2.20 (m, 3H), 2.14-1.94 (m, 3H), 1.81 (dd, J = 21.8, 8.4 Hz, 1H). |
| 1-147 | 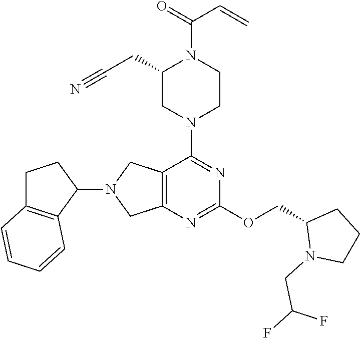 [SMILES: C=CC(=O)N1CCN(c2nc(OC[C@@H]3CCCN3CC(F)F)nc3c2CN(C2CCc4ccccc42)C3)C[C@@H]1CC#N] | B | M + 1 = 578.3 | 78%/ 86% | 1H NMR (400 MHz, CD₃OD) δ 7.42 (d, J = 7.0 Hz, 1H), 7.31-7.15 (m, 3H), 6.78 (s, 1H), 6.27 (d, J = 16.7 Hz, 1H), 6.05-5.72 (m, 2H), 4.99 (s, 1H), 4.54 (dt, J = 11.6, 8.9 Hz, 2H), 4.37-3.99 (m, 6H), 3.82 (s, 2H), 3.62-3.33 (m, 3H), 3.09 (dddd, J = 20.2, 14.3, 7.6, 4.2 Hz, 4H), 2.95- 2.73 (m, 4H), 2.47 (dd, J = 17.1, 8.5 Hz, 1H), 2.36- 2.15 (m, 2H), 2.00 (ddd, J = 16.4, 12.4, 8.5 Hz, 1H), 1.86-1.76 (m, 2H), 1.66 (dt, J = 12.6, 6.0 Hz, 1H). |
| 1-148 | 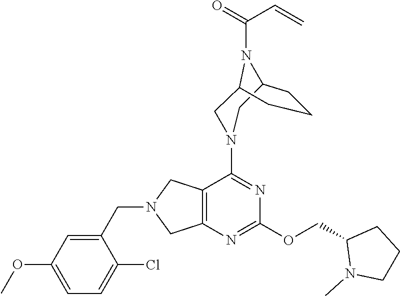 [SMILES: C=CC(=O)N1C2CCCC1CN(c1nc(OC[C@@H]3CCCN3C)nc3c1CN(Cc1cc(OC)ccc1Cl)C3)C2] | A | M + 1 = 567.3 | 0%/ 0% | Intermediate. No NMR |
| 1-149 | 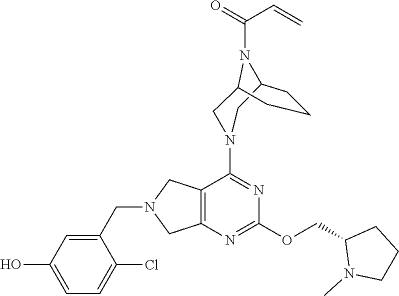 [SMILES: C=CC(=O)N1C2CCCC1CN(c1nc(OC[C@@H]3CCCN3C)nc3c1CN(Cc1cc(O)ccc1Cl)C3)C2] | A | M + 1 = 553.3 | 0%/ 8% | 1H NMR (400 MHz, CD₃OD) δ 7.20 (d, J = 8.7 Hz, 1H), 6.98 (d, J = 3.0 Hz, 1H), 6.83 (dd, J = 16.7, 10.7 Hz, 1H), 6.70 (dd, J = 8.6, 3.0 Hz, 1H), 6.26 (dd, J = 16.8, 1.9 Hz, 1H), 5.80 (dd, J = 10.6, 1.9 Hz, 1H), 4.83-4.20 (m, 9H), 4.11 (d, J = 2.1 Hz, 2H), 3.98 (s, 2H), 3.80 (d, J = 2.1 Hz, 2H), 3.56 (dd, J = 13.2, 3.6 Hz, 1H), 3.16-3.04 (m, 1H), 2.75 (s, 3H), 2.26- 1.69 (m, 10H). |
| 1-150 | 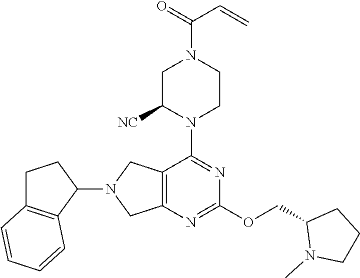 [SMILES: C=CC(=O)N1CCN(c2nc(OC[C@@H]3CCCN3C)nc3c2CN(C2CCc4ccccc42)C3)[C@@H](C#N)C1] | A | M + 1 = 514.3 | 21%/ 31% | 1H NMR (400 MHz, CD₃OD_SPE) δ 8.33 (s, 1H), 7.35 (d, J = 7.3 Hz, 1H), 7.17 (dt, J = 22.3, 7.1 Hz, 2H), 6.75 (dd, J = 16.6, 10.6 Hz, 1H), 6.25 (d, J = 16.8 Hz, 1H), 5.83-5.56 (m, 2H), 4.65 (dd, J = 21.9, 12.7 Hz, 2H), 4.56-4.30 (m, 2H), 4.20 (d, J = 16.7 Hz, 2H), 3.95-3.65 (m, 4H), 3.48 (d, J = 56.0 Hz, 3H), 3.19-2.98 (m, 3H), 2.91 (d, J = 9.0 Hz, 2H), 2.86-2.76 (m, 1H), 2.37- 1.83 (m, 6H). |
| 1-151 | 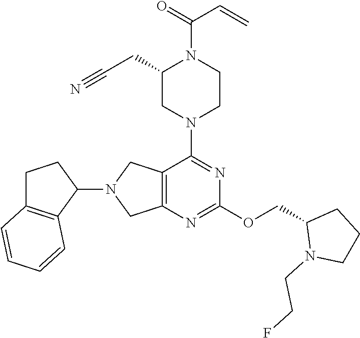 [SMILES: C=CC(=O)N1CCN(c2nc(OC[C@@H]3CCCN3CCF)nc3c2CN(C2CCc4ccccc42)C3)C[C@@H]1CC#N] | A | M + 1 = 560.3 | 81%/ 91% | 1H NMR (400 MHz, CD₃OD) δ 7.42 (d, 1H), 7.30- 7.11 (m, 3H), 6.78 (s, 1H), 6.27 (d,, 1H), 5.82 (d, 1H), 4.99 (s, 1H), 4.67-4.44 (m, 4H), 4.27-4.25 (m, 5H), 3.82 (s, 2H), 3.58-3.34 (m, 2H), 3.24-3.02 (m, 3H), 2.99-2.63 (m, 5H), 2.41-2.39 (m, 1H), 2.34-2.14 (m, 2H), 2.12- 1.89 (m, 2H), 1.87-1.58 (m, 3H). |
| 1-152 | 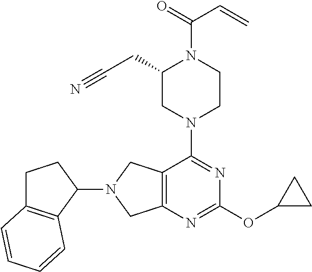 [SMILES: C=CC(=O)N1CCN(c2nc(OC3CC3)nc3c2CN(C2CCc4ccccc42)C3)C[C@@H]1CC#N] | A | M + 1 = 471.2 | 2%/ 19% | 1H NMR (400 MHz, DMSO) δ 8.18 (s, 0H), 7.37 (d, J = 6.7 Hz, 1H), 7.22 (ddt, J = 13.7, 12.1, 6.9 Hz, 3H), 6.82 (d, J = 9.7 Hz, 1H), 6.17 (dd, J = 16.6, 1.9 Hz, 0H), 5.76 (d, J = 10.3 Hz, 0H), 4.89 (s, 0H), 4.45 (dd, J = 11.6, 5.6 Hz, 0H), 4.41-4.07 (m, 2H), 4.00 (d, J = 11.2 Hz, 0H), 3.71 (s, 1H), 3.17 (d, J = 13.7 Hz, 1H), 3.06-2.84 (m, 1H), 2.82-2.74 (m, 0H), 2.21-2.03 (m, 1H), 0.75- 0.58 (m, 1H). |
| 1-153 | 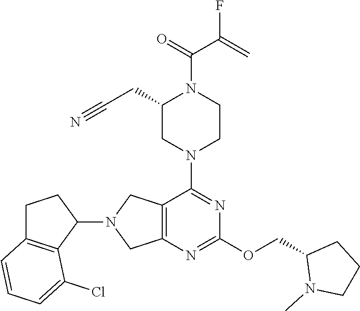 [SMILES: C=C(F)C(=O)N1CCN(c2nc(OC[C@@H]3CCCN3C)nc3c2CN(C2CCc4cccc(Cl)c42)C3)C[C@@H]1CC#N] | A | M + 1 = 580.2 | 46%/ 78% | 1H NMR (400 MHz, CD₃OD) δ 8.53 (s, 1H), 7.23 (s, 3H), 5.56-5.07 (m, 2H), 4.72 (d, J = 14.4 Hz, 1H), 4.62 (d, J = 5.1 Hz, 2H), 4.46-4.33 (m, 1H), 4.25 (d, J = 9.8 Hz, 4H), 3.80 (dd, J = 31.1, 14.1 Hz, 2H), 3.39 (d, J = 36.4 Hz, 3H), 3.19 (s, 4H), 2.87 (d, J = 36.9 Hz, 7H), 2.35 (s, 1H), 2.21 (d, J = 7.4 Hz, 2H), 1.94 (d, J = 49.4 Hz, 3H). |
| 1-154 | 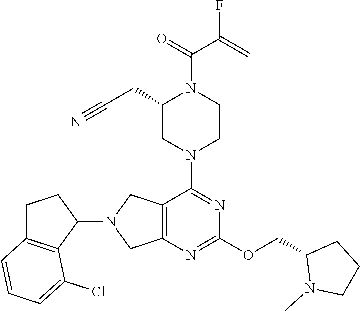 [SMILES: C=C(F)C(=O)N1CCN(c2nc(OC[C@@H]3CCCN3C)nc3c2CN(C2CCc4cccc(Cl)c42)C3)C[C@@H]1CC#N] | A | M + 1 = 580.2 | 52%/ 82% | 1H NMR (400 MHz, CD₃OD) δ 8.56 (s, 1H), 7.29 (d, J = 53.5 Hz, 3H), 5.42-5.17 (m, 2H), 4.62 (s, 3H), 4.42 (d, J = 7.1 Hz, 1H), 4.33-3.98 (m, 4H), 3.81 (s, 2H), 3.43 (s, 3H), 3.29-3.09 (m, 4H), 2.82 (s, 7H), 2.35 (s, 1H), 2.22 (s, 2H), 1.94 (d, J = 49.8 Hz, 3H). |
| 1-155 | 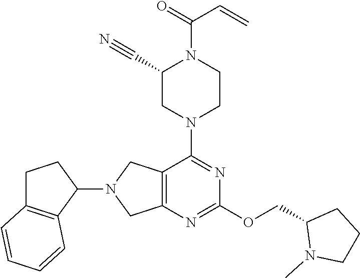 [SMILES: C=CC(=O)N1CCN(c2nc(OC[C@@H]3CCCN3C)nc3c2CN(C2CCc4ccccc42)C3)C[C@@H]1C#N] | K | M + 1 = 514.3 | 66%/ 82% | 1H NMR (400 MHz, CD₃OD) δ = 7.43 (d, 1H), 7.28- 7.17 (m, 3H), 6.79 (dd, 1H), 6.35 (d, 1H), 5.92 (d, 1H), 5.70 (br, 1H), 4.75- 4.73 (m, 6H), 4.59- 4.50 (m, 1H), 4.45-4.18 (m, 6H), 3.84 (s, 2H), 3.38- 3.36 (m, 1H), 3.21-3.19 (m, 1H), 3.12-3.03 (m, 2H), 2.83-2.80 (m, 2H), 2.50 (s, 3H), 2.41-2.02 (m, 5H), 1.82-1.80 (m, 2H), 1.70- 1.69 (m, 1H). |
| 1-156 | 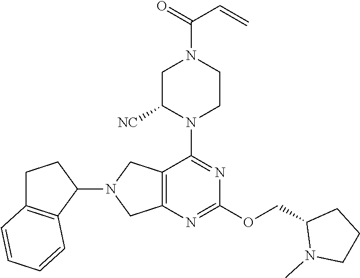 [SMILES: C=CC(=O)N1CCN(c2nc(OC[C@@H]3CCCN3C)nc3c2CN(C2CCc4ccccc42)C3)[C@H](C#N)C1] | K | M + 1 = 514.2 | 11%/ 29% | 1H NMR (400 MHz, CD₃OD) δ 8.41 (s, 2H), 7.43 (d, J = 7.1 Hz, 1H), 7.31-7.19 (m, 3H), 6.83 (dd, J = 16.7, 10.6 Hz, 1H), 6.32 (d, J = 16.7 Hz, 1H), 5.86 (d, J = 10.7 Hz, 1H), 5.76 (s, 1H), 4.80 (t, J = 14.8 Hz, 1H), 4.66-4.46 (m, 3H), 4.27 (q, J = 12.3 Hz, 3H), 3.90 (dt, J = 21.5, 10.5 Hz, 5H), 3.66 (dd, J = 11.0, 6.0 Hz, 1H), 3.25-3.17 (m, 1H), 3.16-3.05 (m, 2H), 3.02 (d, J = 8.5 Hz, 3H), 2.94-2.83 (m, 1H), 2.45-1.95 (m,7H). |
| 1-158 | 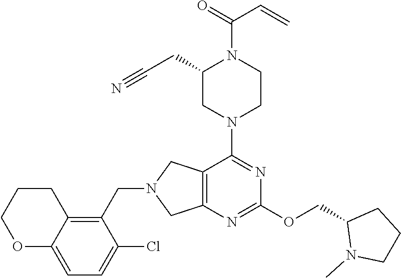 [SMILES: C=CC(=O)N1CCN(c2nc(OC[C@@H]3CCCN3C)nc3c2CN(Cc2c(Cl)ccc4c2CCCO4)C3)C[C@@H]1CC#N] | A | M + 1 = 592.3 | 49%/ 89% | 1H NMR (400 MHz, CD₃OD) δ 7.11 (d, J = 8.8 Hz, 1H), 6.73 (t, J = 18.5 Hz, 2H), 6.27 (d, J = 16.7 Hz, 1H), 5.82 (d, J = 9.5 Hz, 1H), 4.99 (s, 1H), 4.79- 4.44 (m, 2H), 4.32 (s, 2H), 4.21-3.92 (m, 6H), 3.75 (s, 2H), 3.61-3.46 (m, 1H), 3.32 (d, J = 6.1 Hz, 2H), 3.12 (dd, J = 11.4, 9.8 Hz, 2H), 2.97 (s, 5H), 2.50 (s, 4H), 2.15-1.88 (m, 3H), 1.72 (dd, J = 30.2, 24.1 Hz, 3H). |
| 1-159 | 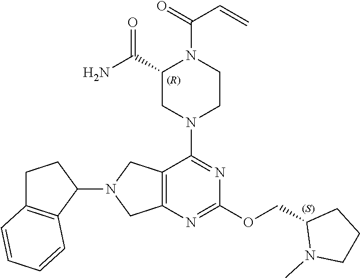 | L | M + 1 = 532.3 | 10%/ 19% | 1H NMR (400 MHz, CD₃OD) δ 7.42 (d, 1H), 7.29- 7.12 (m, 3H), 6.83 (dd, 1H), 6.28 (d, 1H), 5.82 (d, 1H), 5.05 (s, 1H), 4.79-4.76 (m, 1H), 4.64-4.62 (m, 1H), 4.55-4.19 (m, 5H), 4.08 (d, 1H), 3.83 (d, 2H), 3.73 (d, 2H), 3.63-3.51 (m, 2H), 3.39-3.35 (m, 1H), 3.18-3.02 (m, 2H), 3.01 (s, 3H), 2.90-2.80 (m, 1H), 2.34-1.94 (m, 6H). |
| 1-160 | 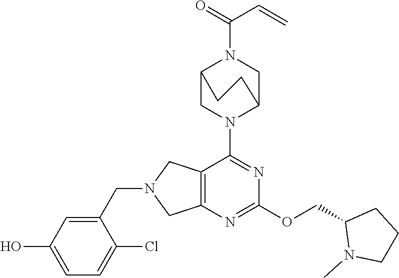 [SMILES: C=CC(=O)N1CC2CCC1CN2c1nc(OC[C@@H]2CCCN2C)nc2c1CN(Cc1cc(O)ccc1Cl)C2] | A | M + 1 = 539.2 | 34%/ 67% | 1H NMR (400 MHz, CD₃OD) δ 7.19 (dd, J = 8.7, 0.8 Hz, 1H), 6.97 (dd, J = 3.0, 1.1 Hz, 1H), 6.81-6.55 (m, 2H), 6.29 (ddd, J = 22.7, 16.8, 2.0 Hz, 1H), 5.79 (ddd, J = 17.1, 10.5, 2.0 Hz, 1H), 4.62-4.35 (m, 3H), 4.20 (s, 2H), 4.03-3.41 (m, 10H), 3.07-2.92 (m, 2H), 2.92-2.82 (m, 3H), 2.33-1.76 (m, 8H). |
| 1-161 | 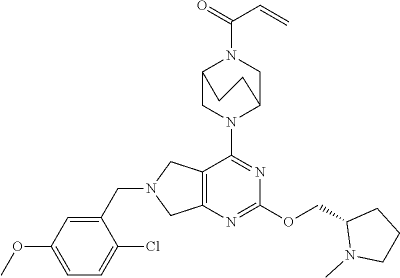 [SMILES: C=CC(=O)N1CC2CCC1CN2c1nc(OC[C@@H]2CCCN2C)nc2c1CN(Cc1cc(OC)ccc1Cl)C2] | A | M + 1 = 553.2 | 0%/ 0% | Intermediate. No NMR |
| 1-162 | 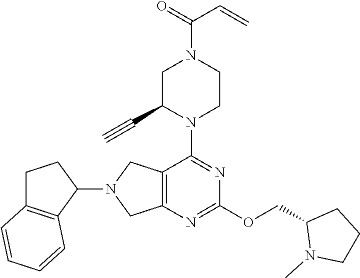 [SMILES: C#C[C@H]1CN(C(=O)C=C)CCN1c1nc(OC[C@@H]2CCCN2C)nc2c1CN(C1CCc3ccccc31)C2] | M | M + 1 = 513.3 | 2%/ 12% | 1H NMR (400 MHz, CD₃OD) δ 8.35 (s, 2H), 7.47- 7.38 (m, 1H), 7.36-7.14 (m, 3H), 6.79 (dd, J = 16.6, 10.1 Hz, 1H), 6.25 (d, J = 16.7 Hz, 1H), 5.79 (d, J = 10.4 Hz, 1H), 5.41 (d, J = 23.3 Hz, 1H), 4.77-4.63 (m, 2H), 4.61-4.46 (m, 2H), 4.32 (d, J = 13.5 Hz, 2H), 4.24-4.03 (m, 2H), 3.97-3.78 (m, 3H), 3.68 (s, 1H), 3.62-3.33 (m, 2H), 3.25-3.16 (m, 1H), 3.15-3.03 (m, 2H), 2.98 (d, J = 27.2 Hz, 3H), 2.93- 2.75 (m, 2H), 2.42-2.27 (m, 2H), 2.24-1.92 (m, 4H). |
| 1-163 | 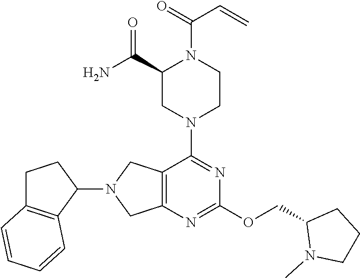 [SMILES: C=CC(=O)N1CCN(c2nc(OC[C@@H]3CCCN3C)nc3c2CN(C2CCc4ccccc42)C3)C[C@H]1C(N)=O] | L | M + 1 = 532.3 | NA/ 55% | 1H NMR (400 MHz, CD₃OD) δ 7.47 (d, J = 7.2 Hz, 1H), 7.26 (dt, J = 15.6, 7.2 Hz, 3H), 6.84 (dd, J = 16.8, 10.6 Hz, 1H), 6.29 (d, J = 16.8 Hz, 1H), 5.83 (d, J = 10.7 Hz, 1H), 5.07 (s, 1H), 4.77 (d, J = 30.9 Hz, 3H), 4.71-4.58 (m, 2H), 4.58-4.40 (m, 2H), 4.33 (t, J = 11.2 Hz, 2H), 4.09 (d, J = 13.6 Hz, 1H), 3.99-3.86 (m, 2H), 3.91- 3.76 (m, 2H), 3.78-3.62 (m, 2H), 3.64-3.51 (m, 1H), 3.49-3.32 (m, 3H), 3.22 (dd, J = 9.8, 6.5 Hz, 1H), 3.18-3.05 (m, 1H), 2.95-2.81 (m, 1H), 2.47- 2.30 (m, 2H), 2.26-1.90 (m, 4H). |
| 1-164 | 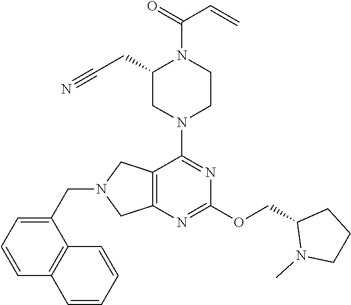 [SMILES: C=CC(=O)N1CCN(c2nc(OC[C@@H]3CCCN3C)nc3c2CN(Cc2cccc4ccccc24)C3)C[C@@H]1CC#N] | A | M + 1 = 552.3 | 93%/ 95% | 1H NMR (400 MHz, CD₃OD) δ 8.45 (s, 1H), 8.35- 8.27 (m, 1H), 7.93-7.80 (m, 2H), 7.58-7.40 (m, 4H), 6.75 (s, 1H), 6.27 (d, J = 16.6 Hz, 1H), 5.81 (d, J = 9.9 Hz, 1H), 4.94 (d, J = 8.6 Hz, 1H), 4.75 (d, J = 11.8 Hz, 2H), 4.45 (dd, J = 12.5, 7.3 Hz, 1H), 4.37 (s, 2H), 4.19 (s, 2H), 4.05 (s, 1H), 3.77 (d, J = 14.1 Hz, 3H), 3.62 (d, J = 4.6 Hz, 1H), 3.48 (d, J = 6.4 Hz, 1H), 3.32 (s, 2H), 3.24- 3.03 (m, 2H), 2.98 (s, 3H), 2.82 (dd, J = 35.1, 15.1 Hz, 2H), 2.34 (dt, J = 14.9, 8.2 Hz, 1H), 2.18-1.94 (m, 3H). |
| 1-165 | 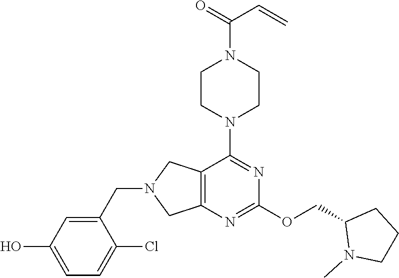 [SMILES: C=CC(=O)N1CCN(c2nc(OC[C@@H]3CCCN3C)nc3c2CN(Cc2cc(O)ccc2Cl)C3)CC1] | G | M + 1 = 513.3 | 88%/ 96% | 1H NMR (400 MHz, CD₃OD) δ 7.17 (d, J = 8.7 Hz, 1H), 6.95 (d, J = 2.9 Hz, 1H), 6.72 (ddd, J = 12.7, 11.6, 6.8 Hz, 2H), 6.22 (dd, J = 16.8, 1.9 Hz, 1H), 5.76 (dd, J = 10.6, 1.9 Hz, 1H), 4.38 (qd, J = 11.6, 5.5 Hz, 2H), 4.14 (s, 2H), 3.95 (s, 2H), 3.76 (d, J = 18.5 Hz, 10H), 3.25 (s, 1H), 3.11 (d, J = 1.7 Hz, 1H), 2.70-2.54 (m, 4H), 2.16 (dq, J = 16.1, 8.0 Hz, 1H), 1.95-1.72 (m, 3H). |
| 1-166 | 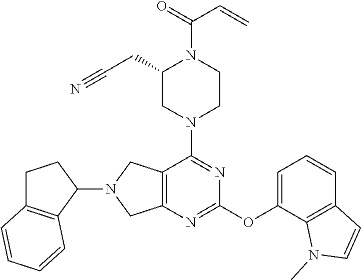 [SMILES: C=CC(=O)N1CCN(c2nc(Oc3cccc4ccn(C)c34)nc3c2CN(C2CCc4ccccc42)C3)C[C@@H]1CC#N] | A | M + 1 = 560.2 | 2%/ 15% | 1H NMR (400 MHz, CD₃OD) δ 7.43 (t, J = 7.2 Hz, 2H), 7.30-7.17 (m, 3H), 7.10-6.99 (m, 2H), 6.84 (d, J = 7.1 Hz, 1H), 6.67 (dd, J = 15.0, 9.1 Hz, 1H), 6.47 (d, J = 3.0 Hz, 1H), 6.20 (d, J = 16.7 Hz, 1H), 5.80-5.72 (m, 1H), 4.70 (s, 1H), 4.56-4.50 (m, 1H), 4.49-4.13 (m, 3H), 3.93-3.73 (m, 7H), 3.37 (s, 1H), 3.13-2.84 (m, 4H), 2.66-2.06 (m, 4H). |
| 1-167 | 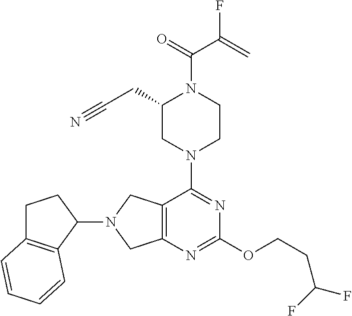 [SMILES: C=C(F)C(=O)N1CCN(c2nc(OCCC(F)F)nc3c2CN(C2CCc4ccccc42)C3)C[C@@H]1CC#N] | B | M + 1 = 527.2 | NA/ 0% | 1H NMR (400 MHz, CD₃OD) δ 7.46 (d, J = 7.2 Hz, 1H), 7.33-7.18 (m, 3H), 6.09 (tt, J = 56.6, 4.7 Hz, 1H), 5.33 (dt, J = 13.7, 12.2 Hz, 2H), 4.94 (s, 1H), 4.66-4.55 (m, 2H), 4.49 (t, J = 6.1 Hz, 2H), 4.37- 4.21 (m, 3H), 4.10 (s, 1H), 3.90 (s, 2H), 3.62 (s, 1H), 3.40 (d, J = 15.1 Hz, 1H), 3.27 (s, 1H), 3.17-3.08 (m, 1H), 3.01-2.82 (m, 3H), 2.39-2.20 (m, 4H). |
| 1-168 | 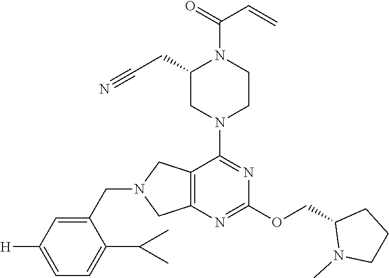 [SMILES: C=CC(=O)N1CCN(c2nc(OC[C@@H]3CCCN3C)nc3c2CN(Cc2ccccc2C(C)C)C3)C[C@@H]1CC#N] | A | M + 1 = 544.3 | 95%/ 97% | 1H NMR (400 MHz, CD₃OD) δ 7.36-7.30 (m, 2H), 7.29-7.24 (m, 1H), 7.14 (td, J = 7.4, 1.3 Hz, 1H), 6.77 (s, 1H), 6.27 (d, J = 16.7 Hz, 1H), 5.82 (d, J = 10.1 Hz, 1H), 5.06-4.94 (m, 1H), 4.64 (d, J = 14.1 Hz, 1H), 4.46-4.31 (m, 2H), 4.27-4.02 (m, 4H), 3.96 (s, 2H), 3.68 (s, 2H), 3.57-3.38 (m, 2H), 3.26- 2.73 (m, 6H), 2.66-2.48 (m, 4H), 2.13 (ddd, J = 16.5, 12.7, 8.2 Hz, 1H), 1.88 (dd, J = 14.4, 7.7 Hz, 2H), 1.85-1.67 (m, 1H), 1.23 (d, J = 6.9 Hz, 6H). |
| 1-169 | 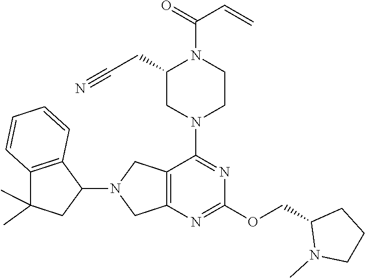 [SMILES: C=CC(=O)N1CCN(c2nc(OC[C@@H]3CCCN3C)nc3c2CN(C2CC(C)(C)c4ccccc42)C3)C[C@@H]1CC#N] | A | M + 1 = 556.3 | 63%/ 70% | 1H NMR (400 MHz, CD₃OD) δ 7.36 (d, J = 7.3 Hz, 1H), 7.30-7.16 (m, 3H), 6.76 (s, 1H), 6.27 (d, J = 16.4 Hz, 1H), 5.81 (d, J = 10.1 Hz, 1H), 4.96 (s, 1H), 4.70 (dd, J = 11.9, 7.4 Hz, 3H), 4.44 (dd, J = 12.3, 7.3 Hz, 1H), 4.34-4.00 (m, 4H), 3.89 (s, 2H), 3.57 (dd, J = 42.5, 20.9 Hz, 3H), 3.37 (s, 2H), 3.06 (s, 3H), 2.93 (s, 2H), 2.78 (s, 1H), 2.29 (dd, J = 13.8, 7.2 Hz, 1H), 2.19 (dd, J = 12.7, 7.1 Hz, 1H), 2.12-1.91 (m, 4H), 1.41 (s, 3H), 1.22 (s, 3H). |
| 1-170 | 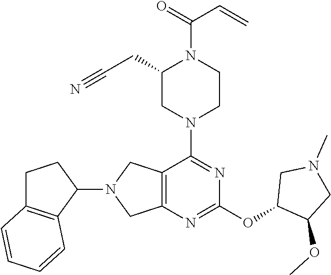 [SMILES: C=CC(=O)N1CCN(c2nc(O[C@@H]3CN(C)C[C@H]3OC)nc3c2CN(C2CCc4ccccc42)C3)C[C@@H]1CC#N] | B | M + 1 = 544.2 | 31%/ 53% | 1H NMR (400 MHz, CD₃OD) δ 7.43 (d, J = 7.1 Hz, 1H), 7.29-7.18 (m, 3H), 6.82 (s, 2H), 6.28 (d, J = 16.8 Hz, 1H), 5.83 (d, J = 9.9 Hz, 1H), 5.27 (s, 1H), 4.99 (s, 1H), 4.53 (dd, J = 12.1, 5.3 Hz, 2H), 4.23 (s, 3H), 4.05 (s, 1H), 3.83 (s, 2H), 3.59-3.43 (m, 2H), 3.38 (s, 3H), 3.27-3.21 (m, 1H), 3.14-3.00 (m, 3H), 2.93-2.82 (m, 3H), 2.79-2.69 (m, 2H), 2.41 (s, 3H), 2.30-2.19 (m, 2H). |
| 1-171 | 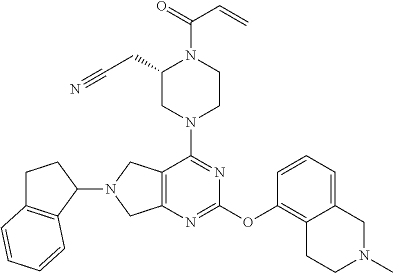 [SMILES: C=CC(=O)N1CCN(c2nc(Oc3cccc4c3CCN(C)C4)nc3c2CN(C2CCc4ccccc42)C3)C[C@@H]1CC#N] | A | M + 1 = 576.3 | 9%/ 29% | 1H NMR (400 MHz, CD₃OD) δ 7.43 (d, J = 6.8 Hz, 1H), 7.28-7.17 (m, 4H), 6.97 (dd, J = 25.2, 7.6 Hz, 2H), 6.75 (s, 1H), 6.23 (d, J = 17.0 Hz, 1H), 5.78 (d, J = 11.0 Hz, 1H), 4.64-4.39 (m, 2H), 4.17 (d, J = 41.0 Hz, 3H), 3.98 (d, J = 11.6 Hz, 1H), 3.86 (s, 2H), 3.76-3.37 (m, 3H), 3.14- 2.82 (m, 5H), 2.64 (d, J = 59.9 Hz, 6H), 2.46 (s, 3H), 2.32-2.18 (m, 2H). |
| 1-172 | 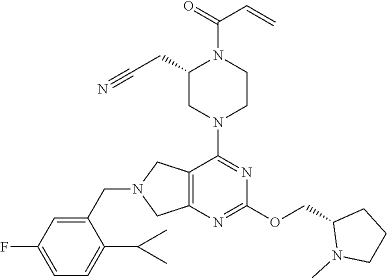 [SMILES: C=CC(=O)N1CCN(c2nc(OC[C@@H]3CCCN3C)nc3c2CN(Cc2cc(F)ccc2C(C)C)C3)C[C@@H]1CC#N] | A | M + 1 = 562.3 | 68%/ 73% | 1H NMR (400 MHz, CD₃OD) δ 8.51 (s, 1H), 7.33 (dd, J = 8.6, 5.8 Hz, 1H), 7.12 (dd, J = 10.0, 2.8 Hz, 1H), 6.99 (td, J = 8.5, 2.8 Hz, 1H), 6.78 (d, J = 11.0 Hz, 1H), 6.28 (d, J = 16.7 Hz, 1H), 5.82 (d, J = 10.1 Hz, 1H), 4.91 (s, 2H), 4.72 (d, J = 10.4 Hz, 3H), 4.49- 4.43 (m, 1H), 4.31-3.99 (m, 4H), 3.96 (s, 2H), 3.74 (s, 2H), 3.70-3.45 (m, 3H), 3.38 (dd, J = 13.6, 6.8 Hz, 2H), 3.13-3.05 (m, 1H), 2.97-2.73 (m, 5H), 2.32 (dt, J = 15.1, 8.2 Hz, 1H), 2.16-1.91 (m, 3H), 1.22 (d, J = 6.8 Hz, 6H). |
| 1-173 | 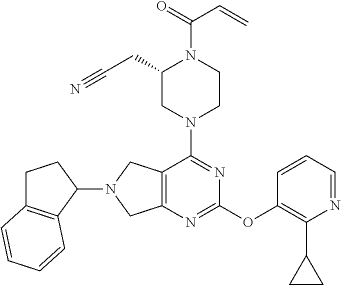 [SMILES: C=CC(=O)N1CCN(c2nc(Oc3cccnc3C3CC3)nc3c2CN(C2CCc4ccccc42)C3)C[C@@H]1CC#N] | A | M + 1 = 548.3 | 6%/ 24% | 1H NMR (400 MHz, CD₃OD) δ 8.31 (dd, J = 38.7, 4.3 Hz, 1H), 7.59-7.47 (m, 1H), 7.43 (d, J = 7.1 Hz, 1H), 7.34-7.16 (m, 4H), 6.82-6.53 (m, 1H), 6.24 (d, J = 16.8 Hz, 1H), 5.79 (d, J = 10.6 Hz, 1H), 4.67- 4.39 (m, 2H), 4.23 (s, 2H), 4.12 (s, 1H), 4.00 (d, J = 10.8 Hz, 1H), 3.88 (s, 2H), 3.47 (s, 1H), 3.22- 2.99 (m, 3H), 2.91-2.82 (m, 1H), 2.67 (d, J = 34.0 Hz, 2H), 2.46-2.00 (m, 4H), 0.98 (ddd, J = 12.0, 8.7, 4.7 Hz, 2H), 0.93-0.84 (m, 2H). |
| 1-174 | 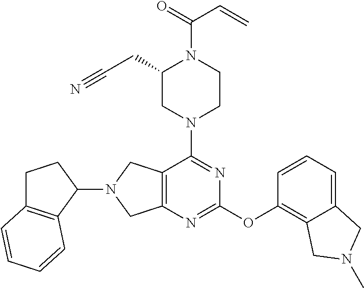 [SMILES: C=CC(=O)N1CCN(c2nc(Oc3cccc4c3CN(C)C4)nc3c2CN(C2CCc4ccccc42)C3)C[C@@H]1CC#N] | A | M + 1 = 562.2 | 9%/ 28% | 1H NMR (400 MHz, CD₃OD) δ 7.43 (d, J = 6.9 Hz, 1H), 7.37-7.20 (m, 4H), 7.15 (d, J = 7.4 Hz, 1H), 6.99 (d, J = 8.0 Hz, 1H), 6.74 (s, 1H), 6.24 (d, J = 16.8 Hz, 1H), 5.79 (d, J = 11.1 Hz, 1H), 4.54 (d, J = 6.7 Hz, 2H), 4.20 (d, J = 29.1 Hz, 3H), 4.02 (d, J = 16.5 Hz, 4H), 3.83 (d, J = 31.9 Hz, 4H), 3.55-3.41 (m, 1H), 3.08 (dt, J = 32.1, 15.8 Hz, 3H), 2.98-2.82 (m, 1H), 2.81-2.41 (m, 5H), 2.25 (dtd, J = 18.5, 13.2, 6.0 Hz, 2H). |
| 1-175 | 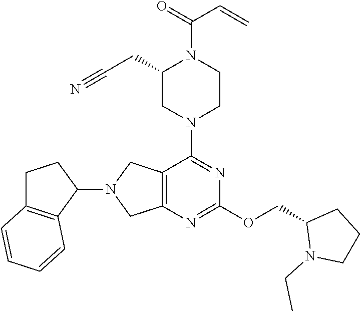 [SMILES: C=CC(=O)N1CCN(c2nc(OC[C@@H]3CCCN3CC)nc3c2CN(C2CCc4ccccc42)C3)C[C@@H]1CC#N] | A | M + 1 = 542.3 | 60%/ 82% | 1H NMR (400 MHz, CD₃OD) δ 7.67 (d, 1H), 7.36- 7.32 (m, 3H), 6.79 (s, 1H), 6.28 (d, 1H), 5.83 (d, 1H), 5.11 (s, 1H), 4.91-4.90 (m, 2H), 4.75-4.65 (m, 3H), 4.52-4.50 (m, 1H), 4.35 (d, 2H), 4.18-3.86 (m, 3H), 3.77-3.66 (m, 1H), 3.55- 3.53 (m, 4H), 3.21-3.20 (m 3H), 3.06-2.73 (m, 3H), 2.69-2.40 (m, 2H), 2.35-2.25 (m, 1H), 2.20- 1.92 (m, 3H), 1.37 (t, 3H). |
| 1-176 | 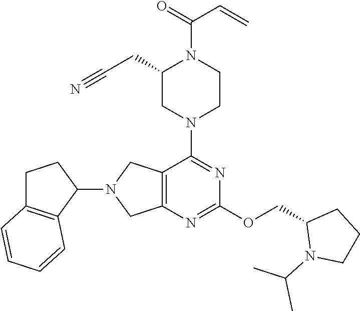 [SMILES: C=CC(=O)N1CCN(c2nc(OC[C@@H]3CCCN3C(C)C)nc3c2CN(C2CCc4ccccc42)C3)C[C@@H]1CC#N] | A | M + 1 = 556.4 | 88%/ 94% | 1H NMR (400 MHz, CD₃OD) δ 7.48 (s, 1H), 7.28- 7.26 (m, 3H), 6.81 (s, 1H), 6.28 (d, 1H), 5.82 (d, 1H), 4.91 (s, 1H), 4.86-4.71 (m, 3H), 4.46-4.43 (m, 3H), 4.16-3.95 (m, 4H), 3.85- 3.73 (m, 1H), 3.63-3.35 (m, 4H), 3.21-3.07 (m, 2H), 2.90-2.79 (m, 4H), 2.41-2.22 (m, 3H), 2.03- 2.02 (m, 3H), 1.40 (d, 3H), 1.34 (d, 3H). |
| 1-180 | 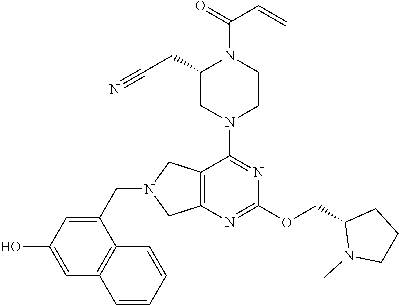 [SMILES: C=CC(=O)N1CCN(c2nc(OC[C@@H]3CCCN3C)nc3c2CN(Cc2cc(O)cc4ccccc24)C3)C[C@@H]1CC#N] | A | M + 1 = 568.2 | 94%/ 96% | 1H NMR (400 MHz, CD₃OD) δ 8.15 (d, J = 8.4 Hz, 1H), 7.66 (d, J = 8.2 Hz, 1H), 7.38 (t, J = 7.5 Hz, 1H), 7.32-7.26 (m, 1H), 7.18 (d, J = 2.1 Hz, 1H), 7.08 (d, J = 2.1 Hz, 1H), 6.75 (s, 1H), 6.27 (d, J = 16.7 Hz, 1H), 5.81 (d, J = 10.2 Hz, 1H), 4.95 (d, J = 13.9 Hz, 1H), 4.84 (d, J = 8.6 Hz, 1H), 4.69 (d, J = 12.2 Hz, 1H), 4.55 (s, 1H), 4.40 (dd, J = 11.8, 6.9 Hz, 1H), 4.29 (s, 2H), 4.17 (s, 2H), 4.08 (d, J = 14.9 Hz, 1H), 3.79 (s, 2H), 3.47 (d, J = 16.2 Hz, 1H), 3.34 (s, 2H), 3.30-3.24 (m, 1H), 3.14 (s, 1H), 2.88 (dd, J = 17.3, 7.6 Hz, 2H), 2.77 (s, 4H), 2.22 (dt, J = 20.9, 8.0 Hz, 1H), 1.97 (dd, J = 18.0, 9.7 Hz, 2H), 1.86 (dd, J = 13.5, 7.6 Hz, 1H). |
| 1-181 | 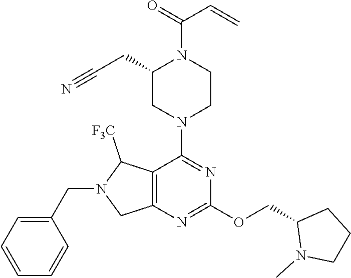 [SMILES: C=CC(=O)N1CCN(c2nc(OC[C@@H]3CCCN3C)nc3c2C(C(F)(F)F)N(Cc2ccccc2)C3)C[C@@H]1CC#N] | N | M + 1 = 570.3 | 0%/ 0% | 1H NMR (400 MHz, CD₃OD) δ 7.34-7.10 (m, 5H), 6.85-6.61 (m, 1H), 6.17 (d, J = 16.7 Hz, 1H), 5.71 (d, J = 10.5 Hz, 1H), 4.70- 4.15 (m, 8H), 4.12-3.84 (m, 3H), 3.78 (d, J = 13.4 Hz, 1H), 3.63-3.30 (m, 2H), 3.09-2.60 (m, 4H), 2.51 (d, J = 11.6 Hz, 4H), 1.99-1.49 (m, 4H). |
| 1-182 | 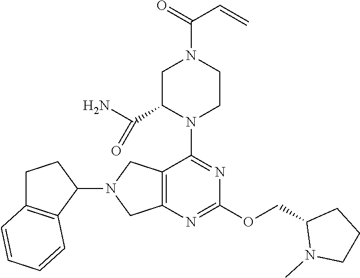 [SMILES: C=CC(=O)N1CCN(c2nc(OC[C@@H]3CCCN3C)nc3c2CN(C2CCc4ccccc42)C3)[C@H](C(N)=O)C1] | A | M + 1 = 532.20 | 7%/ 30% | 1H NMR (400 MHz, CD₃OD) δ = 7.42 (d, 1H), 7.31-7.13 (m, 3H), 6.74- 6.72 (m, 1H), 6.31-6.16 (m, 1H), 5.78 (d, 1H), 5.00- 4.99 (m 1H), 4.66-4.63 (m, 1H), 4.55-4.50 (m, 1H), 4.43-4.42 (m, 1H), 4.30- 4.25 (m, 2H), 4.10-4.07 (m, 2H), 3.87-3.86 (m, 2H), 3.77-3.68 (m, 2H), 3.65-3.63 (m 2H), 3.21-3.19 (m, 2H), 3.07-3.06 (m, 1H), 2.99 (d, 3H), 2.87-2.86 (m IH), 2.23-2.20 (m, 5H), 2.08-1.92 (m, 2H). |
| 1-183 | 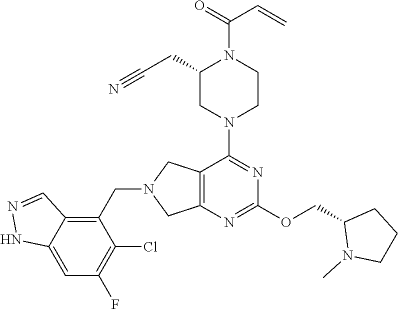 [SMILES: C=CC(=O)N1CCN(c2nc(OC[C@@H]3CCCN3C)nc3c2CN(Cc2c(Cl)c(F)cc4[nH]ncc24)C3)C[C@@H]1CC#N] | A | M + 1 = 594.2 | 63%/ 93% | 1H NMR (400 MHz, CD₃OD) δ 8.48 (s, 1H), 8.06 (s, 1H), 7.88 (d, J = 6.8 Hz, 1H), 6.75 (s, 1H), 6.27 (d, J = 16.7 Hz, 1H), 5.81 (d, J = 10.4 Hz, 1H), 4.93 (s, 1H), 4.73 (d, J = 9.4 Hz, 1H), 4.46 (dd, J = 12.4, 7.3 Hz, 0H), 4.31 (s, 1H), 4.19 (s, 1H), 4.05 (d, J = 10.9 Hz, 0H), 3.84 (s, 1H), 3.57 (ddd, J = 94.2, 60.7, 40.0 Hz, 1H), 3.12 (d, J = 11.1 Hz, 0H), 2.96 (s, 1H), 2.83 (d, J = 44.4 Hz, 1H), 2.33 (dt, J = 15.0, 8.2 Hz, 1H), 2.06 (ttd, J = 22.0, 14.8, 8.0 Hz, 4H). |
| 1-184 | 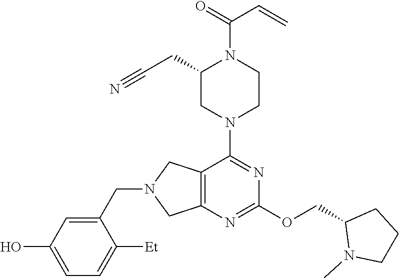 [SMILES: C=CC(=O)N1CCN(c2nc(OC[C@@H]3CCCN3C)nc3c2CN(Cc2cc(O)ccc2CC)C3)C[C@@H]1CC#N] | A | M + 1 = 546.3 | 96%/ 97% | 1H NMR (400 MHz, CD₃OD) δ 8.33 (s, 2H), 7.03 (d, J = 8.3 Hz, 1H), 6.81 (dd, J = 24.1, 7.6 Hz, 2H), 6.67 (dd, J = 8.2, 2.5 Hz, 1H), 6.28 (d, J = 16.6 Hz, 1H), 5.82 (d, J = 10.3 Hz, 1H), 4.93 (d, J = 8.1 Hz, 1H), 4.78 (d, J = 11.2 Hz, 3H), 4.50 (dd, J = 12.5, 7.4 Hz, 1H), 4.05 (s, 4H), 3.89 (s, 2H), 3.84 (d, J = 5.7 Hz, 1H), 3.77 (s, 2H), 3.68 (dt, J = 11.4, 5.8 Hz, 1H), 3.44 (d, J = 63.9 Hz, 2H), 3.21 (d, J = 11.1 Hz, IH), 3.01 (d, J = 14.6 Hz, 3H), 2.95-2.72 (m, 2H), 2.67 (q, J = 7.5 Hz, 2H), 2.36 (dt, J = 14.5, 7.2 Hz, 1H), 2.21-1.96 (m, 3H), 1.17 (t, J = 7.5 Hz, 3H). |
| 1-185 | 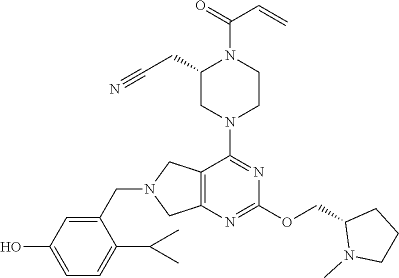 [SMILES: C=CC(=O)N1CCN(c2nc(OC[C@@H]3CCCN3C)nc3c2CN(Cc2cc(O)ccc2C(C)C)C3)C[C@@H]1CC#N] | A | M + 1 = 560.3 | 96%/ 97% | 1H NMR (400 MHz, CD₃OD) δ 8.10 (s, 2H), 6.94 (d, J = 8.5 Hz, 1H), 6.68-6.47 (m, 3H), 6.07 (d, J = 16.6 Hz, 1H), 5.61 (d, J = 10.2 Hz, 1H), 4.74 (s, 1H), 4.61-4.24 (m, 4H), 4.07- 3.78 (m, 4H), 3.71 (s, 2H), 3.63 (dd, J = 12.8, 7.4 Hz, 1H), 3.55 (s, 2H), 3.51-3.45 (m, 1H), 3.32 (s, 1H), 3.17 (d, J = 11.2 Hz, 1H), 3.08-2.95 (m, 2H), 2.82 (s, 3H), 2.62 (ddd, J = 15.3, 13.8, 3.8 Hz, 2H), 2.19-2.11 (m, 1H), 1.98- 1.76 (m, 3H), 0.98 (d, J = 6.8 Hz, 6H). |
| 1-186 | 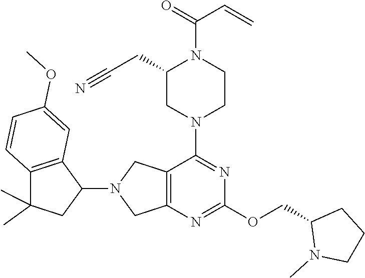 [SMILES: C=CC(=O)N1CCN(c2nc(OC[C@@H]3CCCN3C)nc3c2CN(C2CC(C)(C)c4ccc(OC)cc42)C3)C[C@@H]1CC#N] | A | M + 1 = 586.3 | 14%/ 36% | 1H NMR (400 MHz, CD₃OD) δ 8.53 (s, 0H), 7.11 (d, J = 8.3 Hz, 1H), 6.89 (s, 3H), 6.84 (d, J = 8.2 Hz, 1H), 6.27 (d, J = 16.7 Hz, 1H), 5.82 (d, J = 9.5 Hz, 1H), 4.95 (s, 1H), 4.65 (dd, J = 31.4, 24.5 Hz, 4H), 4.41 (dd, J = 11.5, 7.0 Hz, 1H), 4.30-4.02 (m, 4H), 3.87 (s, 2H), 3.76 (s, 3H), 3.53 (s, 1H), 3.38 (s, 3H), 3.13 (s, 1H), 2.95-2.73 (m, 6H), 2.27-2.15 (m, 2H), 1.98 (dd, J = 11.4. 8.5 Hz, 5H), 1.86 (dd, J = 13.0, 6.8 Hz, 1H), 1.38 (s, 3H), 1.19 (s, 3H). |
| 1-187 | 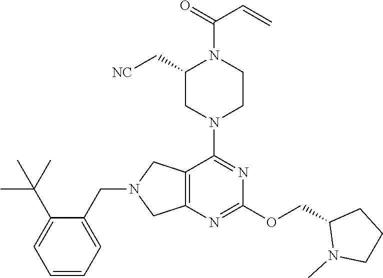 [SMILES: C=CC(=O)N1CCN(c2nc(OC[C@@H]3CCCN3C)nc3c2CN(Cc2ccccc2C(C)(C)C)C3)C[C@@H]1CC#N] | H | M + 1 = 558.3 | 94%/ 95% | 1H NMR (400 MHz, CD₃OD) δ 7.66-7.59 (m, 1H), 7.43 (dd, J = 6.4, 2.9 Hz, 1H), 7.23-7.15 (m, 2H), 6.77 (s, 1H), 6.27 (d, J = 16.9 Hz, 1H), 5.82 (d, J = 10.2 Hz, 1H), 4.97 (s, 1H), 4.62 (d, J = 14.1 Hz, 3H), 4.35 (d, J = 5.8 Hz, 2H), 4.19 (s, 2H), 4.16 (s, 2H), 4.04 (s, 1H), 3.74 (s, 2H), 3.51 (d, J = 21.5 Hz, 1H), 3.14-3.08 (m, 1H), 2.93- 2.76 (m, 3H), 2.53 (s, 3H), 2.40 (dd, J = 17.9, 9.0 Hz, 1H), 2.22-2.00 (m, 2H), 1.83 (td, J = 12.8, 7.6 Hz, 2H), 1.70 (dt, J = 19.9, 7.3 Hz, 1H), 1.46 (d, J = 10.6 Hz, 9H). |
| 1-188 | 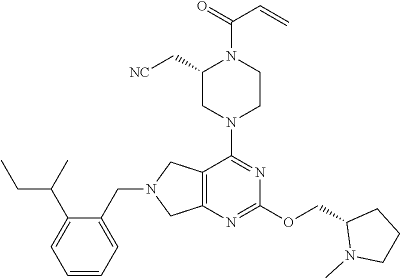 [SMILES: C=CC(=O)N1CCN(c2nc(OC[C@@H]3CCCN3C)nc3c2CN(Cc2ccccc2C(C)CC)C3)C[C@@H]1CC#N] | A | M + 1 = 558.3 | 47%/ 79% | 1H NMR (400 MHz, CD₃OD) δ 7.32 (d, J = 7.5 Hz, 1H), 7.30-7.23 (m, 2H), 7.17-7.11 (m, 1H), 6.89- 6.72 (m, 1H), 6.27 (d, J = 16.8 Hz, 1H), 5.82 (d, J = 10.2 Hz, 1H), 4.98 (s, 1H), 4.82-4.43 (m, 2H), 4.41- 4.31 (m, 2H), 4.18-4.05 (m, 3H), 3.94 (dd, J = 32.3, 12.7 Hz, 2H), 3.67 (s, 2H), 3.48 (ddd, J = 42.8, 22.2, 12.0 Hz, 2H), 3.26- 3.06 (m, 3H), 2.85 (ddd, J = 21.4, 18.3, 11.5 Hz, 3H), 2.56 (s, 3H), 2.46 (dd, J = 17.9, 9.0 Hz, 1H), 2.15- 2.06 (m, 1H), 1.89-1.81 (m, 2H), 1.75-1.59 (m, 3H), 1.20 (d, J = 6.8 Hz, 3H), 0.82 (t, J = 7.4 Hz, 3H). |
| 1-189 | 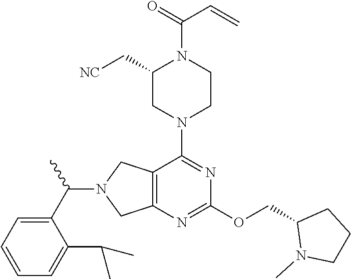 [SMILES: C=CC(=O)N1CCN(c2nc(OC[C@@H]3CCCN3C)nc3c2CN(C(C)c2ccccc2C(C)C)C3)C[C@@H]1CC#N] | A | M + 1 = 558.2 | 57%/ 61% | 1H NMR (400 MHz, CD₃OD) δ 7.48 (d, J = 7.5 Hz, 1H), 7.30 (d, J = 7.0 Hz, 1H), 7.18 (dt, J = 15.4, 6.6 Hz, 2H), 6.74 (s, 1H), 6.25 (d, J = 16.7 Hz, 1H), 5.79 (d, J = 9.8 Hz, 1H), 4.91 (d, J = 2.5 Hz, 1H), 4.56 (d, J = 23.5 Hz, 2H), 4.37- 4.28 (m, 2H), 4.20-4.08 (m, 3H), 3.99 (d, J = 11.4 Hz, 2H), 3.63 (s, 2H), 3.47 (d, J = 4.9 Hz, 2H), 3.16- 3.07 (m, 2H), 2.91-2.81 (m, 2H), 2.53 (s, 3H), 2.42 (dd, J = 17.8, 9.0 Hz, 1H), 2.19-1.99 (m, 2H), 1.87- 1.78 (m, 2H), 1.69 (dt, J = 20.1, 7.3 Hz, 1H), 1.42 (d, J = 6.4 Hz, 3H), 1.26- 1.20 (m, 6H). |
| 1-190 | 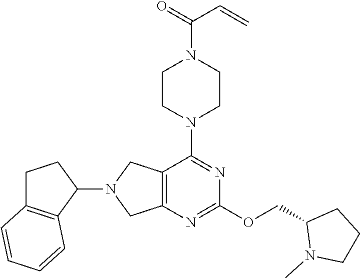 [SMILES: C=CC(=O)N1CCN(c2nc(OC[C@@H]3CCCN3C)nc3c2CN(C2CCc4ccccc42)C3)CC1] | A | M + 1 = 489.2 | 6%/ 26% | 1H NMR (400 MHz, CD₃OD) δ 8.36 (s, 1H), 7.42 (d, J = 7.1 Hz, 1H), 7.23 (dt, J = 14.8, 7.1 Hz, 3H), 6.78 (dd, J = 16.8, 10.6 Hz, 1H), 6.25 (dd, J = 16.8, 1.8 Hz, 1H), 5.79 (dd, J = 10.6, 1.8 Hz, 1H), 4.67 (dd, J = 12.5, 3.2 Hz, 1H), 4.58-4.52 (m, 1H), 4.47 (dd, J = 12.5, 7.2 Hz, 1H), 4.24 (q, J = 11.7 Hz, 2H), 3.85 (s, 2H), 3.76 (s, 8H), 3.69- 3.61 (m, 1H), 3.24-3.04 (m, 3H), 3.00 (d, J = 9.3 Hz, 3H), 2.93-2.82 (m, 1H), 2.40-2.26 (m, 2H), 2.25-2.11 (m, 2H), 2.10- 1.93 (m, 2H). |
| 1-191 | 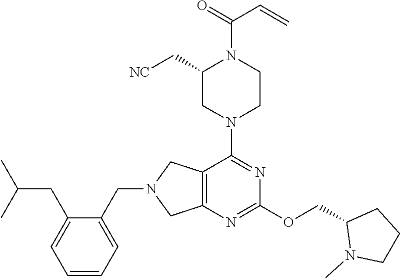 [SMILES: C=CC(=O)N1CCN(c2nc(OC[C@@H]3CCCN3C)nc3c2CN(Cc2ccccc2CC(C)C)C3)C[C@@H]1CC#N] | A | M + 1 = 558.3 | 52%/ 73% | 1H NMR (400 MHz, CD₃OD) δ 7.40-7.36 (m, 1H), 7.23-7.14 (m, 3H), 6.79 (dd, J = 31.0, 19.3 Hz, 1H), 6.27 (d, J = 16.6 Hz, 1H), 5.81 (d, J = 10.1 Hz, 1H), 4.98 (s, 1H), 4.80-4.46 (m, 2H), 4.35 (d, J = 6.3 Hz, 2H), 4.24-4.01 (m, 4H), 3.93 (s, 2H), 3.69 (s, 2H), 3.51 (d, J = 11.6 Hz, 1H), 3.38 (d, J = 13.5 Hz, 1H), 3.13 (dd, J = 9.9, 5.2 Hz, 1H), 2.87 (dt, J = 16.9, 8.5 Hz, 3H), 2.62 (d, J = 7.2 Hz, 2H), 2.55 (s, 3H), 2.44 (dd, J = 17.9, 9.0 Hz, 1H), 2.14-2.04 (m, 1H), 1.91- 1.80 (m, 3H), 1.76-1.67 (m, 1H), 0.92 (d, J = 6.6 Hz, 6H). |
| 1-192 | 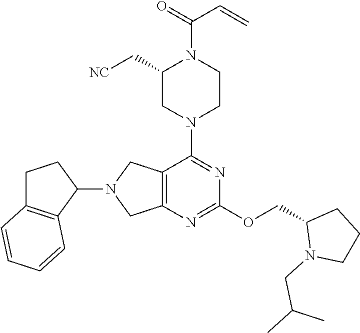 [SMILES: C=CC(=O)N1CCN(c2nc(OC[C@@H]3CCCN3CC(C)C)nc3c2CN(C2CCc4ccccc42)C3)C[C@@H]1CC#N] | A | M + 1 = 570.3 | 53%/ 65% | 1H NMR (400 MHz, CD₃OD) δ 7.42 (d, J = 7.2 Hz, 1H), 7.22 (dq, J = 13.7, 7.0 Hz, 3H), 6.78 (s, 1H), 6.27 (d, J = 16.7 Hz, 1H), 5.82 (d, J = 10.6 Hz, 1H), 4.97 (s, 1H), 4.81-4.45 (m, 3H), 4.45-3.89 (m, 6H), 3.83 (s, 2H), 3.51 (d, J = 26.0 Hz, 2H), 3.25- 2.99 (m, 3H), 2.87 (dd, J = 18.4. 11.4 Hz, 5H), 2.34- 2.18 (m, 3H), 1.92 (d, J = 81.5 Hz, 5H), 0.93 (t, J = 6.0 Hz, 6H). |
| 1-193 | 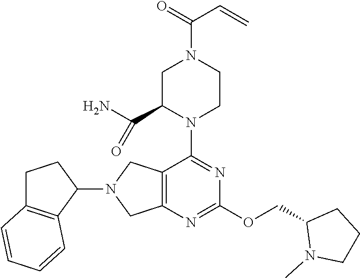 [SMILES: C=CC(=O)N1CCN(c2nc(OC[C@@H]3CCCN3C)nc3c2CN(C2CCc4ccccc42)C3)[C@@H](C(N)=O)C1] | A | M + 1 = 532.2 | 7%/ 31% | 1H NMR (400 MHz, CD₃OD) δ 8.38 (s, 1.5H), 7.42 (d, J = 6.9 Hz, 1H), 7.28- 7.12 (m, 3H), 6.84-6.62 (m, 1H), 6.22 (dd, J = 16.2, 7.6 Hz, 1H), 5.76 (d, J = 10.4 Hz, 1H), 5.04 (s, 1H), 4.71-4.42 (m, 4H), 4.33 (dd, J = 23.9, 11.1 Hz, 2H), 4.09 (d, J = 52.9 Hz, 2H), 3.88 (s, 2H), 3.71 (d, J = 49.5 Hz, 4H), 3.30 (s, 1H), 3.18 (d, J = 6.5 Hz, 1H), 3.07 (dd, J = 15.3, 7.5 Hz, 1H), 2.98 (d, J = 8.5 Hz, 3H), 2.92-2.82 (m, 1H), 2.30 (dd, J = 14.1, 7.0 Hz, 2H), 2.12 (ddd, J = 46.3, 17.6, 10.8 Hz, 3H), 1.92 (s, 1H). |
| 1-194 | 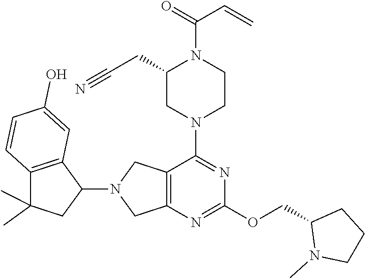 [SMILES: C=CC(=O)N1CCN(c2nc(OC[C@@H]3CCCN3C)nc3c2CN(C2CC(C)(C)c4ccc(O)cc42)C3)C[C@@H]1CC#N] | A | M + 1 = 572.2 | 32%/ 32% | 1H NMR (400 MHz, CD₃OD) δ 7.03 (d, J = 8.2 Hz, 1H), 6.86-6.67 (m, 3H), 6.28 (d, J = 16.9 Hz, 1H), 5.82 (d, J = 10.3 Hz, 1H), 4.97 (s, 1H), 4.79 (s, 2H), 4.68 (s, 1H), 4.48 (s, 1H), 4.31 (s, 2H), 4.08 (s, 1H), 3.89 (d, J = 28.7 Hz, 3H), 3.68 (s, 1H), 3.54 (s, 1H), 3.39 (s, 2H), 3.21 (dd, J = 14.6, 7.3 Hz, 2H), 3.04 (s, 3H), 2.84 (d, J = 44.6 Hz, 2H), 2.43-2.32 (m, 1H), 2.17 (d, J = 5.9 Hz, 2H), 2.10-1.92 (m, 3H), 1.37 (s, 3H), 1.18 (s, 3H). |
| 1-195 | 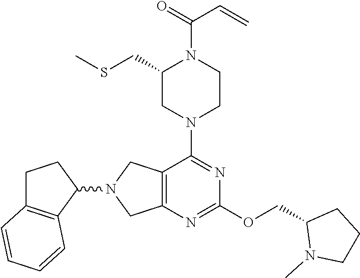 [SMILES: C=CC(=O)N1CCN(c2nc(OC[C@@H]3CCCN3C)nc3c2CN(C2CCc4ccccc42)C3)C[C@@H]1CSC] | B | M + 1 = 549.2 | 0%/ 2% | 1H NMR (400 MHz, CD₃OD) δ 7.46-7.39 (m, 1H), 7.22 (dq, J = 14.5, 7.1 Hz, 3H), 6.87-6.68 (m, 1H), 6.23 (d, J = 16.6 Hz, 1H), 5.78 (d, J = 10.6 Hz, 1H), 4.79 (d, J = 25.7 Hz, 1H), 4.61 (d, J = 8.9 Hz, 1H), 4.55-4.49 (m, 1H), 4.33 (d, J = 5.9 Hz, 4H), 4.20 (d, J = 11.3 Hz, 1H), 4.02 (d, 13.9 Hz, 1H), 3.83 (s, 2H), 3.52-3.33 (m, 1H), 3.22 (d, J = 23.4 Hz, 1H), 3.17-3.00 (m, 3H), 2.91-2.70 (m, 3H), 2.57 (d, J = 8.4 Hz, 1H), 2.51 (s, 3H), 2.41-2.05 (m, 7H), 1.82 (td, J = 8.0, 3.0 Hz, 2H), 1.68 (dt, J = 18.7, 6.9 Hz, 1H). |
| 1-196 | 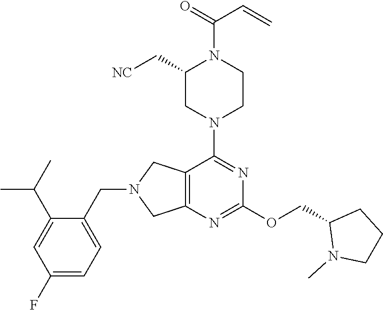 [SMILES: C=CC(=O)N1CCN(c2nc(OC[C@@H]3CCCN3C)nc3c2CN(Cc2ccc(F)cc2C(C)C)C3)C[C@@H]1CC#N] | A | M + 1 = 562.3 | 41%/ 52% | 1H NMR (400 MHz, CD₃OD) δ 8.48 (s, 1H), 7.34 (dd, J = 8.4, 6.1 Hz, 1H), 7.05 (dd, J = 10.8, 2.7 Hz, 1H), 6.85 (ddd, J = 30.9, 18.3, 7.2 Hz, 2H), 6.28 (d, J = 16.6 Hz, 1H), 5.82 (d, J = 10.2 Hz, 1H), 4.95 (d, J = 11.0 Hz, 1H), 4.74 (d, 11.8 Hz, 2H), 4.48 (dd, J = 12.5, 7.4 Hz, 1H), 4.08 (d, J = 31.7 Hz, 4H), 3.94 (s, 2H), 3.80-3.68 (m, 3H), 3.67-3.60 (m, 1H), 3.56-3.33 (m, 3H), 3.23- 3.03 (m, 2H), 3.00 (d, J = 10.6 Hz, 3H), 2.94-2.71 (m, 2H), 2.33 (dt, J = 19.2, 7.3 Hz, 1H), 2.06 (dddd, J = 26.2, 18.9, 12.2, 5.7 Hz, 3H), 1.22 (d, J = 6.8 Hz, 6H). |
| 1-197 | 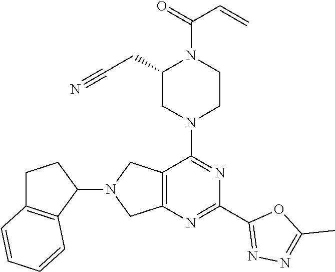 [SMILES: C=CC(=O)N1CCN(c2nc(-c3nnc(C)o3)nc3c2CN(C2CCc4ccccc42)C3)C[C@@H]1CC#N] | O | M + 1 = 497.3 | 24%/ 52% | 1H NMR (400 MHz, CD₃OD) δ 7.44 (d, 1H), 7.24- 7.22 (m, 3H), 6.80 (s, 1H), 6.28 (d, 1H), 5.83 (d, 1H), 5.04 (s, 1H), 4.58- 4.55 (m, 2H), 4.34-4.32 (m, 3H), 4.00 (s, 2H), 3.48- 3.42 (m, 3H), 3.09-3.06 (m, 2H), 2.98-2.79 (m, 3H), 2.64 (s, 3H), 2.38-2.17 (m, 2H). |
| 1-198 | 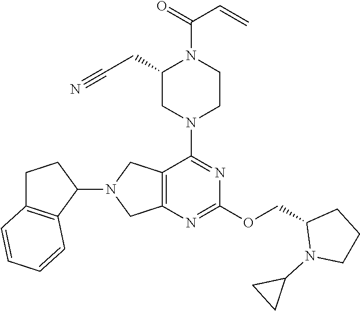 [SMILES: C=CC(=O)N1CCN(c2nc(OC[C@@H]3CCCN3C3CC3)nc3c2CN(C2CCc4ccccc42)C3)C[C@@H]1CC#N] | Q | M + 1 = 554.3 | 15%/ 18% | 1H NMR (400 MHz, CD₃OD) δ 7.44-7.17 (m, 4H), 6.79 (dd, J = 30.3, 10.6 Hz, 1H), 6.27 (d, J = 16.8 Hz, 1H), 5.93-5.80 (m, 1H), 4.99 (s, 1H), 4.83-4.38 (m, 4H), 4.34-4.12 (m, 3H), 4.10-3.64 (m, 5H), 3.64-3.36 (m, 1H), 3.27-2.95 (m, 8H), 2.93-2.67 (m, 1H), 2.18 (dddt, J = 34.4, 26.7, 9.3, 6.1 Hz, 4H), 1.81 (ddd, J = 28.6, 13.4, 6.8 Hz, 3H), 0.79-0.39 (m, 4H). |
| 1-199 | 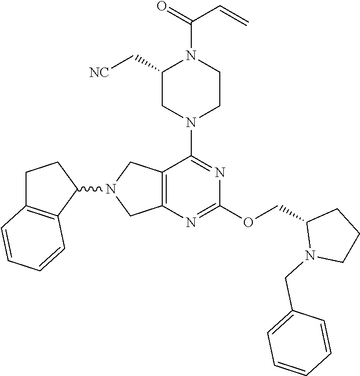 [SMILES: C=CC(=O)N1CCN(c2nc(OC[C@@H]3CCCN3Cc3ccccc3)nc3c2CN(C2CCc4ccccc42)C3)C[C@@H]1CC#N] | Q | M + 1 = 604.3 | 62%/ 74% | 1H NMR (400 MHz, CD₃OD) δ 7.58-7.31 (m, 6H), 7.23 (dt, J = 15.6, 7.0 Hz, 3H), 6.76 (s, 1H), 6.26 (d, J = 16.6 Hz, 1H), 5.81 (d, J = 10.2 Hz, 1H), 4.61 (d, J = 12.6 Hz, 4H), 4.53-4.37 (m, 1H), 4.27 (d, J = 13.5 Hz, 3H), 4.16-3.78 (m, 4H), 3.64-3.33 (m, 3H), 3.25 (d, J = 7.2 Hz, 1H), 3.18-3.00 (m, 1H), 2.94- 2.68 (m, 3H), 2.44-2.15 (m, 3H), 2.00 (t, J = 34.4 Hz, 3H). |
| 1-200 | 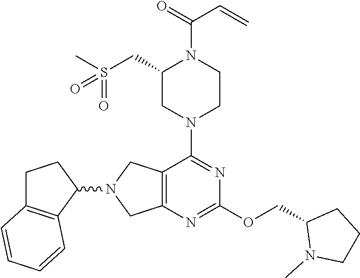 [SMILES: C=CC(=O)N1CCN(c2nc(OC[C@@H]3CCCN3C)nc3c2CN(C2CCc4ccccc42)C3)C[C@@H]1CS(C)(=O)=O] | A | M + 1 = 581.2 | 0%/ 2% | 1H NMR (400 MHz, CD₃OD) δ 7.43 (d, J = 7.1 Hz, 1H), 7.23 (ddt, J = 14.8, 13.4, 6.7 Hz, 3H), 6.75 (dd, J = 16.8, 10.7 Hz, 1H), 6.30-6.21 (m, 1H), 5.84- 5.76 (m, 1H), 5.21-5.11 (m, 1H), 4.74-4.61 (m, 1H), 4.54-4.47 (m, 1H), 4.40-4.16 (m, 5H), 4.11- 4.00 (m, 1H), 3.86-3.79 (m, 2H), 3.50 (dd, J = 24.2, 22.6 Hz, 2H), 3.39 (s, 2H), 3.19-2.96 (m, 6H), 2.93- 2.77 (m, 2H), 2.55 (s, 3H), 2.45 (d, J = 8.6 Hz, 1H), 2.34-2.17 (m, 2H), 2.11 (dt, J = 20.7, 8.2 Hz, 1H), 1.85 (dd, J = 15.3, 7.7 Hz, 2H), 1.71 (dt, J = 20.1, 7.3 Hz, 1H). |
| 1-201 | 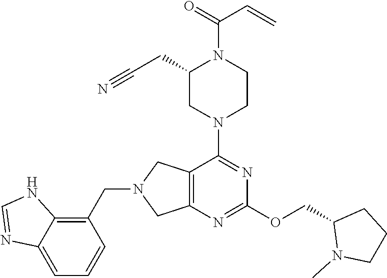 [SMILES: C=CC(=O)N1CCN(c2nc(OC[C@@H]3CCCN3C)nc3c2CN(Cc2cccc4nc[nH]c24)C3)C[C@@H]1CC#N] | A | M + 1 = 542.3 | 79%/ 94% | 1H NMR (400 MHz, CD₃OD) δ 8.16 (s, 1H), 7.57 (s, 1H), 7.27 (d, J = 8.0 Hz, 2H), 6.76 (s, 1H), 6.26 (d, J = 16.4 Hz, 1H), 5.82 (s, 1H), 4.94 (d, J = 19.9 Hz, 1H), 4.59 (d, J = 12.9 Hz, 2H), 4.39-4.23 (m, 4H), 4.16 (s, 3H), 3.79 (s, 2H), 3.48 (d, J = 1.6 Hz, 1H), 3.41-3.32 (m, 1H), 3.12 (dd, J = 7.8, 6.1 Hz, 2H), 2.86 (dd, J = 16.6, 7.4 Hz, 3H), 2.51 (s, 3H), 2.40 (s, 1H), 2.13-2.03 (m, 1H), 1.82 (s, 2H), 1.70 (d, J = 12.2 Hz, 1H). |
| 1-202 | 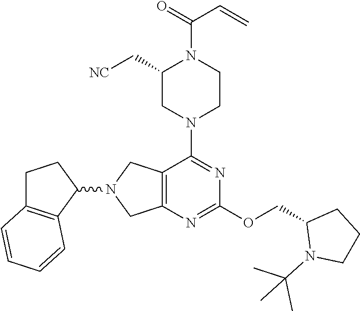 [SMILES: C=CC(=O)N1CCN(c2nc(OC[C@@H]3CCCN3C(C)(C)C)nc3c2CN(C2CCc4ccccc42)C3)C[C@@H]1CC#N] | B | M + 1 = 570.3 | 82%/ 89% | 1H NMR (400 MHz, CD₃OD) δ 7.42 (d, J = 7.1 Hz, 1H), 7.29-7.17 (m, 3H), 6.79 (d, J = 12.8 Hz, 1H), 6.27 (d, J = 16.5 Hz, 1H), 5.82 (d, J = 10.5 Hz, 1H), 4.99 (s, 1H), 4.74 (s, 1H), 4.52 (dd, J = 11.6, 5.5 Hz, 2H), 4.20 (d, J = 11.4 Hz, 4H), 4.05 (s, 2H), 3.82 (s, 2H), 3.57-3.37 (m, 2H), 3.16-2.97 (m, 3H), 2.88 (ddd, J = 21.2, 13.3, 5.7 Hz, 4H), 2.31-2.17 (m, 2H), 1.94-1.81 (m, 3H), 1.74 (s, 1H), 1.17 (s, 9H). |
| 1-203 | 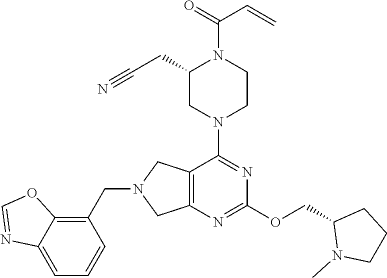 [SMILES: C=CC(=O)N1CCN(c2nc(OC[C@@H]3CCCN3C)nc3c2CN(Cc2cccc4ncoc24)C3)C[C@@H]1CC#N] | H | M + 1 = 543.3 | 11%/ 52% | 1H NMR (400 MHz, CD₃OD) δ 8.51 (s, 1H), 7.75- 7.68 (m, 1H), 7.52 (d, J = 7.0 Hz, 1H), 7.43 (t, J = 7.7 Hz, 1H), 6.77 (s, 1H), 6.28 (d, J = 16.5 Hz, 1H), 5.82 (d, J = 10.1 Hz, 1H), 4.98 (s, 1H), 4.60 (d, J = 9.5 Hz, 3H), 4.36 (t, J = 10.0 Hz, 2H), 4.24 (d, J = 23.9 Hz, 4H), 3.81 (s, 2H), 3.62-3.47 (m, 1H), 3.37 (s, 1H), 3.14 (d, J = 3.5 Hz, 2H), 2.89 (dd, J = 16.7, 7.7 Hz, 3H), 2.55 (s, 3H), 2.45 (d, J = 8.6 Hz, 1H), 2.15- 2.06 (m, 1H), 1.92-1.81 (m, 2H), 1.77-1.67 (m, 1H). |
| 1-204 | 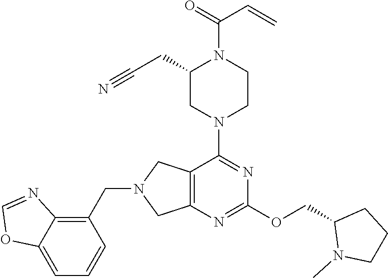 [SMILES: C=CC(=O)N1CCN(c2nc(OC[C@@H]3CCCN3C)nc3c2CN(Cc2cccc4ocnc24)C3)C[C@@H]1CC#N] | H | M + 1 = 543.3 | 46%/ 84% | 1H NMR (400 MHz, CD₃OD) δ 8.49 (s, 1H), 7.62 (dd, J = 7.8, 1.3 Hz, 1H), 7.50-7.43 (m, 2H), 6.77 (s, 1H), 6.27 (d, J = 16.8 Hz, 1H), 5.81 (d, J = 10.6 Hz, 1H), 4.98 (s, 1H), 4.60 (d, J = 14.0 Hz, 1H), 4.37- 4.28 (m, 4H), 4.18 (d, J = 31.5 Hz, 4H), 3.80 (s, 2H), 3.50 (d, J = 16.7 Hz, 1H), 3.39 (s, 1H), 3.20- 3.06 (m, 2H), 2.98-2.81 (m, 3H), 2.56 (s, 3H), 2.52- 2.45 (m, 1H), 2.11 (ddd, J = 16.6, 12.6, 8.3 Hz, 1H), 1.86 (dd, J = 15.2, 8.0 Hz, 2H), 1.77-1.68 (m, 1H). |
| 1-206 | 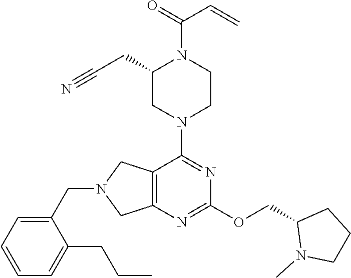 [SMILES: C=CC(=O)N1CCN(c2nc(OC[C@@H]3CCCN3C)nc3c2CN(Cc2ccccc2CCC)C3)C[C@@H]1CC#N] | A | M + 1 = 544.2 | 86%/ 94% | 1H NMR (400 MHz, CD₃OD) δ 8.38 (s, 1H), 7.36 (d, J = 7.2 Hz, 1H), 7.25-7.13 (m, 3H), 6.75 (s, 1H), 6.27 (d, J = 15.9 Hz, 1H), 5.82 (d, J = 10.6 Hz, 1H), 4.77 (d, J = 11.5 Hz, 2H), 4.45 (dd, J = 12.6, 7.3 Hz, 1H), 4.13 (s, 2H), 4.07 (s, 2H), 3.95 (s, 2H), 3.78 (s, 1H), 3.73 (s, 2H), 3.63 (s, 2H), 3.48 (s, 2H), 3.16 (d, J = 25.7 Hz, 2H), 3.00 (s, 2H), 2.88 (d, J = 15.1 Hz, 2H), 2.76-2.69 (m, 2H), 2.39-2.30 (m, 1H), 2.20- 2.11 (m, 1H), 2.10-1.93 (m, 3H), 1.63 (td, J = 14.9, 7.4 Hz, 2H), 0.97 (t, J = 7.3 Hz, 3H). |
| 1-207 | 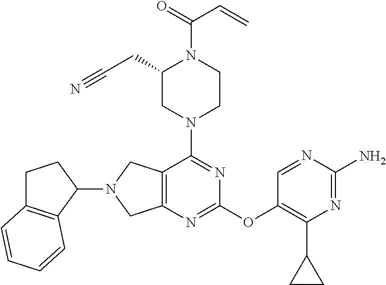 [SMILES: C=CC(=O)N1CCN(c2nc(Oc3cnc(N)nc3C3CC3)nc3c2CN(C2CCc4ccccc42)C3)C[C@@H]1CC#N] | A | M + 1 = 564.2 | 16%/ 50% | 1H NMR (400 MHz, CD₃OD) δ 7.90 (s, 1H), 7.41 (d, J = 7.1 Hz, 1H), 7.33-7.11 (m, 3H), 6.74 (s, 1H), 6.23 (d, J = 16.4 Hz, 1H), 5.78 (d, J = 10.1 Hz, 1H). 4.90 (s, 1H), 4.79-4.64 (m, 1H), 4.53 (dd, J = 11.8, 6.9 Hz, 1H), 4.23 (s, 2H), 4.06 (d, J = 12.7 Hz, 2H), 3.87 (s, 2H), 3.42 (d, J = 36.2 Hz, 1H), 3.23-3.16 (m, 1H), 3.14-3.03 (m, 2H), 2.84 (dd, J = 14.4, 7.4 Hz, 1H), 2.73-2.40 (m, 2H), 2.31-2.16 (m, 2H), 1.98-1.88 (m, 1H), 1.05 (dd, J = 8.9, 4.5 Hz, 2H), 0.90 (dd, J = 8.0, 3.6 Hz, 2H). |
| 1-208 | 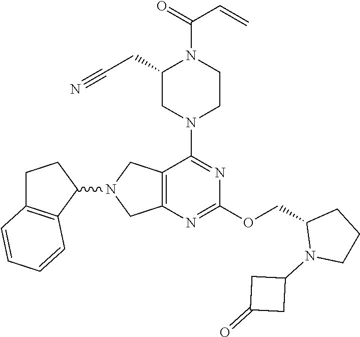 [SMILES: C=CC(=O)N1CCN(c2nc(OC[C@@H]3CCCN3C3CC(=O)C3)nc3c2CN(C2CCc4ccccc42)C3)C[C@@H]1CC#N] | Q | M + 1 = 582.3 | 0%/ 18% | 1H NMR (400 MHz, CD₃OD) δ 7.87 (d, J = 12.6 Hz, 1H), 7.41 (d, J = 7.0 Hz, 1H), 7.33-7.07 (m, 2H), 6.76 (s, 1H), 6.26 (d, J = 16.6 Hz, 1H), 5.80 (d, J = 9.8 Hz, 1H), 5.14-4.86 (m, 3H), 4.72-4.32 (m, 4H), 4.32-3.97 (m, 4H), 3.82 (s, 2H), 3.46 (s, 1H), 3.17-2.98 (m, 2H), 2.96- 2.68 (m, 3H), 2.38-2.08 (m, 3H), 2.04 (d, J = 16.0 Hz, 4H). |
| 1-209 | 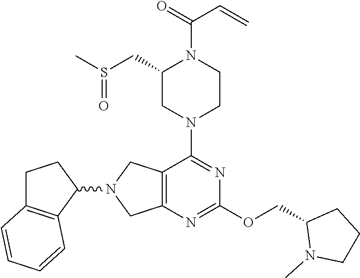 [SMILES: C=CC(=O)N1CCN(c2nc(OC[C@@H]3CCCN3C)nc3c2CN(C2CCc4ccccc42)C3)C[C@@H]1CS(C)=O] | A | M + 1 = 565.2 | 0%/ 0% | 1H NMR (400 MHz, CD₃OD) δ 8.29 (s, 2H), 7.46 (d, J = 6.9 Hz, 1H), 7.32-7.19 (m, 3H), 6.79 (dd, J = 36.4, 23.5 Hz, 1H), 6.28 (dd, J = 25.4, 15.5 Hz, 1H), 5.82 (d, J = 10.6 Hz, 1H), 5.17 (s, 1H), 4.87-4.44 (m, 5H), 4.40-4.01 (m, 4H), 3.96-3.80 (m, 3H), 3.72-3.39 (m, 3H), 3.22 (dd, J = 17.7, 9.7 Hz, 2H), 3.14-3.01 (m, 5H), 2.93-2.86 (m, 1H), 2.72 (d, J = 27.9 Hz, 3H), 2.39-1.97 (m, 6H). |
| 1-210 | 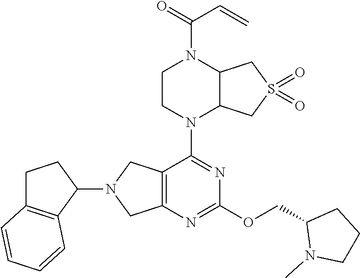 [SMILES: C=CC(=O)N1CCN(c2nc(OC[C@@H]3CCCN3C)nc3c2CN(C2CCc4ccccc42)C3)C2CS(=O)(=O)CC21] | A | M + 1 = 579.3 | 0%/ 0% | 1H NMR (400 MHz, CD₃OD) δ 7.42 (d, J = 7.3 Hz, 1H), 7.32-7.16 (m, 3H), 6.76 (dd, J = 16.7, 10.6 Hz, 1H), 6.30 (dd, J = 16.8, 1.9 Hz, 1H), 5.83 (dd, J = 10.6, 1.9 Hz, 1H), 5.52-5.53 (m, 1H), 5.37-5.24 (m, 1H), 4.73 (dddd, J = 12.6, 7.5, 3.6, 1.6 Hz, 1H), 4.55 (dddd, J = 12.5, 8.8, 7.0, 1.5 Hz, 2H), 4.37-4.29 (m, 1H), 4.14-3.45 (m, 12H), 3.16-3.01 (m, 3H), 2.95- 2.81 (m, 4H), 2.44-1.85 (m, 6H). |
| 1-212 | 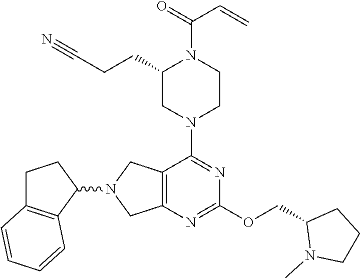 [SMILES: C=CC(=O)N1CCN(c2nc(OC[C@@H]3CCCN3C)nc3c2CN(C2CCc4ccccc42)C3)C[C@@H]1CCC#N] | B | M + 1 = 542.3 | 0%/ 5% | 1H NMR (400 MHz, CD₃OD) δ 8.38 (s, 1H), 7.43 (d, J = 7.2 Hz, 1H), 7.32-7.17 (m, 3H), 6.77 (dd, J = 16.7, 10.9 Hz, 1H), 6.35- 6.17 (m, 1H), 5.80 (d, J = 10.5 Hz, 1H), 4.84 (s, 1H), 4.66 (dd, J = 12.5, 3.2 Hz, 1H), 4.61-4.33 (m, 4H), 4.31-4.15 (m, 3H), 4.05 (d, J = 13.6 Hz, 1H), 3.88 (d, J = 1.6 Hz, 2H), 3.81 (ddd, J = 15.6, 7.8, 3.2 Hz, 1H), 3.70-3.63 (m, 1H), 3.50 (dd, J = 17.0, 7.0 Hz, 1H), 3.24-3.05 (m, 3H), 3.02 (s, 3H), 2.92-1-2.81 (m, 1H), 2.56-2.42 (m, 2H), 2.41-1-2.28 (m, 2H), 2.24-1.87 (m, 6H). |
| 1-213 | 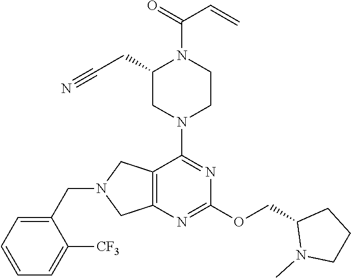 [SMILES: C=CC(=O)N1CCN(c2nc(OC[C@@H]3CCCN3C)nc3c2CN(Cc2ccccc2C(F)(F)F)C3)C[C@@H]1CC#N] | A | M + 1 = 570.2 | 94%/ 95% | 1H NMR (400 MHz, CD₃OD) δ 7.83 (d, J = 7.8 Hz, 1H), 7.68 (t, J = 11.8 Hz, 1H), 7.62 (t, J = 7.6 Hz, 1H), 7.44 (t, J = 7.6 Hz, 1H), 6.27 (d, J = 16.8 Hz, 1H), 5.81 (d, J = 10.2 Hz, 1H), 5.09-4.90 (m, 1H), 4.82-4.40 (m, 2H), 4.40 (s, 2H), 4.10 (dd, J = 39.3, 19.8 Hz, 6H), 3.74 (d, J = 13.7 Hz, 2H), 3.50 (d, J = 21.6 Hz, 1H), 3.46-3.32 (m, 1H), 3.19-3.02 (m, 2H), 2.99-2.73 (m, 3H), 2.46-2.31 (m, 1H), 2.20- 1.99 (m, 1H), 1.94-1.75 (m, 2H), 1.76 (s, 1H). |
| 1-214 | 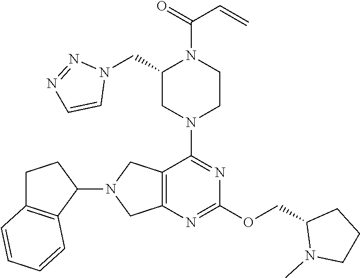 [SMILES: C=CC(=O)N1CCN(c2nc(OC[C@@H]3CCCN3C)nc3c2CN(C2CCc4ccccc42)C3)C[C@@H]1Cn1ccnn1] | B | M + 1 = 570.3 | 0%/ 2% | 1H NMR (400 MHz, CD₃OD) δ 7.96 (d, J = 32.2 Hz, 1H), 7.70 (s, 1H), 7.43 (d, J = 6.7 Hz, 1H), 7.24 (tt, J = 14.2, 7.0 Hz, 3H), 6.71- 6.25 (m, 1H), 6.05 (dd, J = 42.2, 16.9 Hz, 1H), 5.64 (dd, J = 72.6, 10.5 Hz, 1H), 5.14 (s, 1H), 4.70 (dd, J = 25.5, 17.8 Hz, 3H), 4.59-4.34 (m, 4H), 4.27- 4.00 (m, 3H), 3.80 (t, J = 29.3 Hz, 3H), 3.36 (dd, J = 14.0, 3.5 Hz, 3H), 3.23 (d, J = 17.3 Hz, 1H), 3.09 (dd, J = 15.5, 7.6 Hz, 1H), 2.95-2.62 (m, 5H), 2.27 (ddd, J = 19.4, 11.9, 5.8 Hz, 3H), 2.06-1.80 (m, 3H). |
| 1-215 | 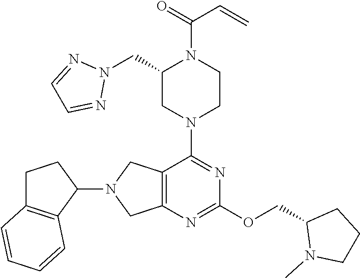 [SMILES: C=CC(=O)N1CCN(c2nc(OC[C@@H]3CCCN3C)nc3c2CN(C2CCc4ccccc42)C3)C[C@@H]1Cn1nccn1] | B | M + 1 = 570.3 | 0%/ 7% | 1H NMR (400 MHz, CD₃OD) 7.63 (s, 2H), 7.44 (d, J = 6.5 Hz, 1H), 7.25 (tt, J = 14.6, 7.2 Hz, 3H), 6.71- 6.26 (m, 1H), 6.05 (dd, J = 32.3, 17.1 Hz, 1H), 5.64 (dd, J = 65.1, 10.2 Hz, 1H), 5.20 (s, 1H), 4.77-4.62 (m, 3H), 4.52 (dd, J = 27.9, 22.4 Hz, 3H), 4.38 (s, 1H), 4.31-4.00 (m, 3H), 3.96-3.82 (m, 3H), 3.69 (dd, J = 10.7, 6.0 Hz, 1H), 3.39 (d, J = 14.0 Hz, 2H), 3.24 (dt, J = 11.3, 8.0 Hz, 2H), 3.14-3.07 (m, 1H), 3.04 (s, 3H), 2.94-2.84 (m, 1H), 2.36 (ddd, J = 20.7, 10.3, 6.6 Hz, 2H), 2.28- 2.14 (m, 2H), 2.14-1.97 (m, 2H). |
| 1-216 | 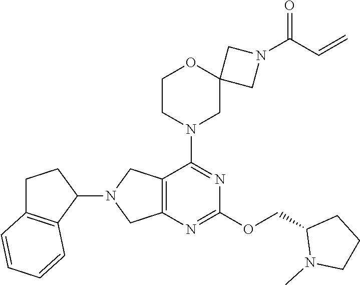 [SMILES: C=CC(=O)N1CC2(C1)CN(c1nc(OC[C@@H]3CCCN3C)nc3c1CN(C1CCc4ccccc41)C3)CCO2] | A | M + 1 = 531.3 | 18%/ 54% | 1H NMR (CD₃OD) δ 7.41 (d, J = 7.3 Hz, 1H), 7.32- 7.11 (m, 3H), 6.38 (ddd, J = 17.0, 10.0, 3.2 Hz, 1H), 6.28 (dd, J = 17.0, 2.1 Hz, 1H), 5.78 (dt, J = 10.1, 2.2 Hz, 1H), 4.72-4.41 (m, 3H), 4.30-4.10 (m, 4H), 4.11-3.59 (m, 12H), 3.30- 3.05 (m, 2H), 3.01 (d, J = 2.6 Hz, 3H), 2.87 (ddd, J = 15.9, 8.5, 5.4 Hz, 1H), 2.40-1.89 (m, 6H). |
| 1-217 | 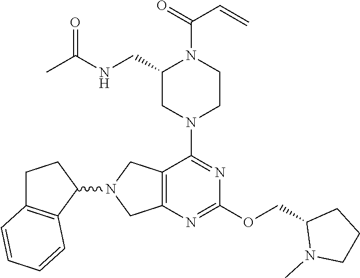 [SMILES: C=CC(=O)N1CCN(c2nc(OC[C@@H]3CCCN3C)nc3c2CN(C2CCc4ccccc42)C3)C[C@@H]1CNC(C)=O] | B | M + 1 = 560.2 | 0%/ 0% | 1H NMR (400 MHz, CD₃OD) δ 8.42 (s, 1H), 7.44- 7.38 (m, 1H), 7.29-7.17 (m, 3H), 6.73 (dd, J = 16.5, 10.6 Hz, 1H), 6.24 (dd, J = 31.2, 16.8 Hz, 1H), 5.77 (dd, J = 10.6, 1.6 Hz, 1H), 4.64 (dd, J = 12.5, 3.3 Hz, 1H), 4.58-4.47 (m, 7H), 4.45-4.18 (m, 1H), 3.98 (d, J = 13.7 Hz, 1H), 3.83 (d, J = 15.4 Hz, 1H), 3.76 (d, J = 5.2 Hz, 1H), 3.63 (dd, J = 11.3, 5.0 Hz, 1H), 3.49-3.34 (m, 2H), 3.24- 3.02 (m, 4H), 3.00 (s, 3H), 2.91-2.83 (m, 1H), 2.38-1.94 (m, 7H), 1.84 (d, J = 2.9 Hz, 3H). |
| 1-218 | 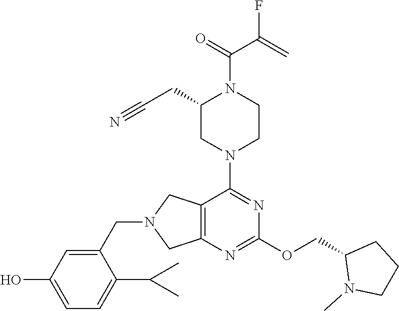 [SMILES: C=C(F)C(=O)N1CCN(c2nc(OC[C@@H]3CCCN3C)nc3c2CN(Cc2cc(O)ccc2C(C)C)C3)C[C@@H]1CC#N] | A | M + 1 = 578.3 | 95%/ 94% | 1H NMR (400 MHz, CD₃OD) δ 8.47 (s, 1H), 7.14 (d, J = 8.5 Hz, 1H), 6.79 (d, J = 2.6 Hz, 1H), 6.71 (dd, J = 8.4, 2.6 Hz, 1H), 5.40-5.23 (m, 2H), 4.75 (dd, J = 12.6, 2.9 Hz, 3H), 4.47 (dd, J = 12.6, 7.3 Hz, 1H), 4.30-4.00 (m, 4H), 3.89 (s, 2H), 3.83-3.77 (m, 1H), 3.72 (s, 2H), 3.64 (dd, J = 11.3, 6.9 Hz, 1H), 3.36 (d, J = 16.8 Hz, 2H), 3.20 (ddd, J = 21.8, 18.5, 7.0 Hz, 3H), 3.04-2.83 (m, 5H), 2.40-2.31 (m, 1H), 2.21-2.13 (m, 1H), 2.02 (ddd, J = 18.9, 14.0, 6.3 Hz, 2H), 1.19 (d, J = 6.8 Hz, 6H). |
| 1-219 | 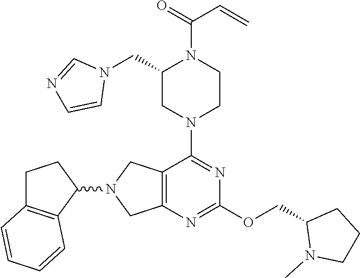 [SMILES: C=CC(=O)N1CCN(c2nc(OC[C@@H]3CCCN3C)nc3c2CN(C2CCc4ccccc42)C3)C[C@@H]1Cn1ccnc1] | B | M + 1 = 569.3 | 0%/ 1% | 1H NMR (400 MHz, CD₃OD) δ 8.33 (s, 2H), 7.83 (t, J = 46.1 Hz, 1H), 7.45 (d, J = 6.6 Hz, 1H), 7.32- 7.14 (m, 4H), 7.06 (t, J = 17.4 Hz, 1H), 6.87-6.26 (m, 1H), 6.26-5.88 (m, 1H), 5.67 (dd, J = 80.0, 10.3 Hz, 1H), 5.01 (d, J = 49.8 Hz, 1H), 4.42 (ddd, J = 32.2, 27.5, 11.0 Hz, 10H), 3.92 (s, 2H), 3.86-3.80 (m, 1H), 3.75-3.55 (m, 2H), 3.33 (s, 2H), 3.23 (dd, J = 8.1, 3.1 Hz, 1H), 3.15- 3.03 (m, 4H), 2.91 (d, J = 5.8 Hz, 1H), 2.36 (dd, J = 13.7, 7.3 Hz, 2H), 2.28- 2.06 (m, 3H), 2.01 (d, J = 22.4 Hz, 1H). |
| 1-220 | 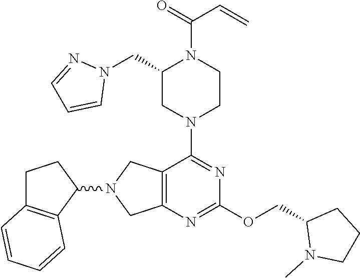 [SMILES: C=CC(=O)N1CCN(c2nc(OC[C@@H]3CCCN3C)nc3c2CN(C2CCc4ccccc42)C3)C[C@@H]1Cn1cccn1] | B | M + 1 = 569.3 | 0%/ 2% | 1H NMR (400 MHz, CD₃OD) δ 8.38 (s, 1.5H), 7.58- 7.39 (m, 3H), 7.25 (dt, J = 15.8, 7.3 Hz, 3H), 6.73- 6.23 (m, 2H), 6.20-5.99 (m, 1H), 5.65 (dd, J = 75.5, 9.9 Hz, 1H), 5.09 (s, 1H), 4.71-4.18 (m, 10H), 4.03 (d, J = 13.4 Hz, 1H), 3.87 (s, 2H), 3.83-3.77 (m, 1H), 3.68-3.63 (m, 1H), 3.37 (d, J = 12.5 Hz, 1H), 3.20 (dt, J = 11.4, 8.0 Hz, 2H), 3.14-3.06 (m, 1H), 3.02 (s, 3H), 2.92-2.84 (m, 1H), 2.34 (ddd, J = 21.2, 10.7, 6.7 Hz, 2H), 2.24- 2.14 (m, 2H), 2.12-1.95 (m, 2H). |
| 1-221 | 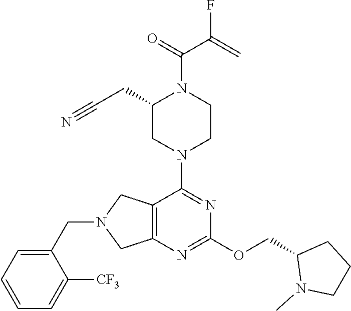 [SMILES: C=C(F)C(=O)N1CCN(c2nc(OC[C@@H]3CCCN3C)nc3c2CN(Cc2ccccc2C(F)(F)F)C3)C[C@@H]1CC#N] | A | M + 1 = 588.2 | 62%/ 92% | 1H NMR (400 MHz, CD₃OD) δ 7.84 (d, J = 7.8 Hz, 1H), 7.70 (d, J = 7.9 Hz, 1H), 7.63 (t, J = 7.5 Hz, 1H), 7.45 (t, J = 7.7 Hz, 1H), 5.37-5.24 (m, 2H), 4.90 (s, 1H), 4.67-4.59 (m, 1H), 4.39-4.31 (m, 2H), 4.20 (d, J = 9.3 Hz, 3H), 4.12 (s, 2H), 3.76 (s, 2H), 3.38 (s, 1H), 3.27 (d, J = 1.7 Hz, 2H), 3.13 (s, 1H), 2.94 (d, J = 7.0 Hz, 4H), 2.55 (s, 2H), 2.43 (dd, J = 17.8, 8.7 Hz, 1H), 2.25- 2.04 (m, 1H), 1.84 (dd, J = 7.5, 5.1 Hz, 2H). |
| 1-222 | 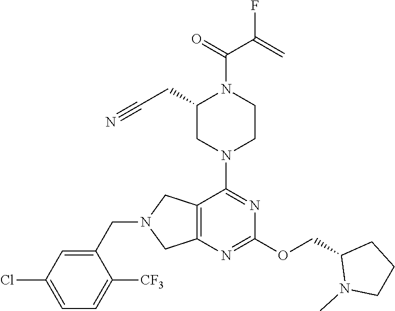 [SMILES: C=C(F)C(=O)N1CCN(c2nc(OC[C@@H]3CCCN3C)nc3c2CN(Cc2cc(Cl)ccc2C(F)(F)F)C3)C[C@@H]1CC#N] | A | M + 1 = 622.2 | 3%/ 24% | 1H NMR (400 MHz, CD₃OD) δ 7.88 (d, J = 0.9 Hz, 1H), 7.69 (d, J = 8.5 Hz, 1H), 7.47 (d, J = 8.3 Hz, 1H), 5.40-5.20 (m, 2H), 4.86-4.65 (m, 2H), 4.58 (dd, J = 11.9, 4.0 Hz, 1H), 4.41 (dd, J = 11.9, 6.9 Hz, 1H), 4.14 (dd, J = 37.0, 19.5 Hz, 6H), 3.79 (s, 2H), 3.65-3.32 (m, 4H), 3.22 (s, 1H), 2.96-2.74 (m, 6H), 2.29-2.19 (m, 1H), 2.03-1.85 (m, 3H). |
| 1-223 | 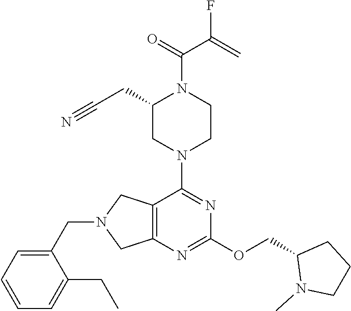 [SMILES: C=C(F)C(=O)N1CCN(c2nc(OC[C@@H]3CCCN3C)nc3c2CN(Cc2ccccc2CC)C3)C[C@@H]1CC#N] | A | M + 1 = 530.3 | 94%/ 95% | 1H NMR (400 MHz, CD₃OD) δ 7.35 (d, J = 7.3 Hz, 1H), 7.21 (t, J = 3.6 Hz, 2H), 7.18-7.13 (m, 1H), 6.76 (s, 1H), 6.27 (d, J = 16.2 Hz, 1H), 5.81 (d, J = 10.7 Hz, 1H), 5.01 (s, 1H), 4.61 (d, J = 16.1 Hz, 2H), 4.40-4.29 (m, 2H), 4.11 (s, 3H), 3.94 (s, 2H), 3.70 (s, 2H), 3.48 (s, 2H), 3.13 (s, 2H), 2.83 (ddd, J = 22.7, 16.0, 7.7 Hz, 5H), 2.55 (s, 3H), 2.45 (d, J = 8.9 Hz, 1H), 2.10 (dt, J = 20.7, 8.4 Hz, 1H), 1.88-1.79 (m, 2H), 1.77-1.66 (m, 1H), 1.21 (t, J = 7.6 Hz, 3H). |
| 1-224 | 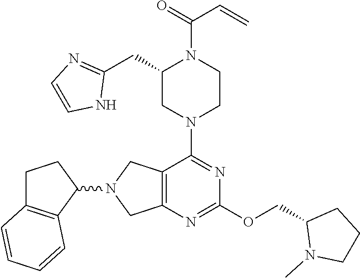 [SMILES: C=CC(=O)N1CCN(c2nc(OC[C@@H]3CCCN3C)nc3c2CN(C2CCc4ccccc42)C3)C[C@@H]1Cc1ncc[nH]1] | I | M + 1 = 569.3 | 0%/ 0% | 1H NMR (400 MHz, CD₃OD) δ 8.34 (s, 2H), 7.40 (t, J = 6.0 Hz, 1H), 7.30-7.18 (m, 2H), 7.00 (s, 1H), 6.59 (d, J = 78.9 Hz, 1H), 6.10 (s, 1H), 5.68 (d, J = 40.7 Hz, 1H), 5.11 (s, 1H), 4.66 (s, 1H), 4.60-4.47 (m, 2H), 4.44 (dd, J = 12.3, 7.1 Hz, 1H), 4.32 (s, 2H), 4.18 (d, J = 10.4 Hz, 1H), 4.01 (dd, J = 20.5, 11.5 Hz, 1H), 3.94-3.72 (m, 3H), 3.66 (ddd, J = 11.8, 6.9, 4.9 Hz, 2H), 3.49-3.32 (m, 2H), 3.20 (dt, J = 11.6, 8.1 Hz, 2H), 3.14-2.97 (m, 5H), 2.93-2.82 (m, 1H), 2.33 (ddd, J = 26.3, 14.6, 8.1 Hz, 2H), 2.23- 2.12 (m, 2H), 2.11-1.90 (m, 2H). |
| 1-225 | 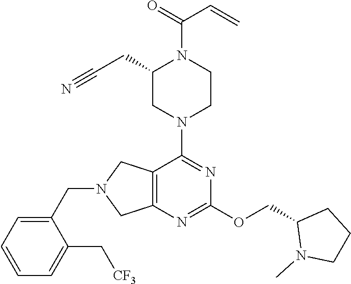 [SMILES: C=CC(=O)N1CCN(c2nc(OC[C@@H]3CCCN3C)nc3c2CN(Cc2ccccc2CC(F)(F)F)C3)C[C@@H]1CC#N] | H | M + 1 = 584.2 | 77%/ 93% | 1H NMR (400 MHz, CD₃OD) δ 7.58-7.42 (m, 1H), 7.44-7.26 (m, 3H), 6.29 (d, J = 16.8 Hz, 1H), 5.83 (d, J = 10.7 Hz, 1H), 5.00 (s, 1H), 4.87-4.42 (m, 2H), 4.36 (d, J = 5.7 Hz, 2H), 4.09 (dd, J = 49.7, 21.1 Hz, 6H), 3.80 (q, J = 11.3 Hz, 2H), 3.67 (s, 2H), 3.61- 3.45 (m, 1H), 3.37 (s, 1H), 3.24-3.06 (m, 1H), 3.00-2.76 (m, 3H), 2.54 (d, J = 3.8 Hz, 3H), 2.41 (d, J = 8.6 Hz, 1H), 2.17-2.01 (m, 1H), 1.85 (s, 2H), 1.80-1.64 (m, 1H). |
| 1-227 | 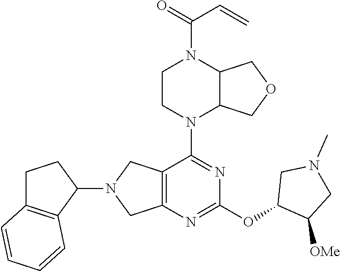 [SMILES: C=CC(=O)N1CCN(c2nc(O[C@@H]3CN(C)C[C@H]3OC)nc3c2CN(C2CCc4ccccc42)C3)C2COCC21] | A | M + 1 = 547.3 | 0%/ 0% | 1H NMR (DMSO-d6) δ 7.40- 7.12 (m, 4H), 6.77 (dd, J = 16.6, 10.5 Hz, 1H), 6.15 (dd, J = 16.6, 2.4 Hz, 1H), 5.73 (dd, J = 10.4, 2.3 Hz, 1H), 5.11-4.84 (m, 3H), 4.45 (m, 1H), 4.25- 3.28 (m, 14H), 3.23 (d, J = 2.2 Hz, 3H), 3.06-2.66 (m, 4H), 2.29 (m, 1H), 2.20 (s, 3H), 2.18-2.05 (m, 2H). |
| 1-228 | 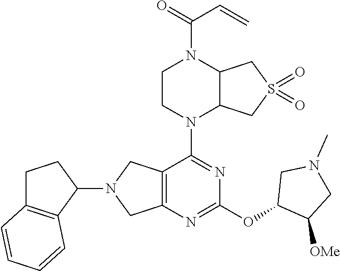 [SMILES: C=CC(=O)N1CCN(c2nc(O[C@@H]3CN(C)C[C@H]3OC)nc3c2CN(C2CCc4ccccc42)C3)C2CS(=O)(=O)CC21] | A | M + 1 = 595.3 | 0%/ 0% | 1H NMR (DMSO-d6) δ 7.34- 7.08 (m, 4H), 6.71 (tt, J = 10.5, 6.5 Hz, 1H), 6.12 (dd, J = 16.7, 2.3 Hz, 1H), 5.70 (dd, J = 10.4, 2.2 Hz, 1H), 5.34-4.90 (m, 4H), 4.45-4.35 (m, 1H), 4.19- 3.12 (m, 15H), 2.96-2.67 (m, 4H), 2.29-1.93 (m, 7H). |
| 1-229 | 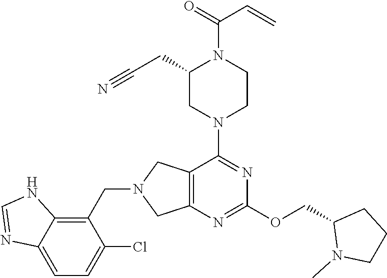 [SMILES: C=CC(=O)N1CCN(c2nc(OC[C@@H]3CCCN3C)nc3c2CN(Cc2c(Cl)ccc4nc[nH]c24)C3)C[C@@H]1CC#N] | A | M + 1 = 576.2 | 94%/ 95% | 1H NMR (400 MHz, CD₃OD) δ 8.19 (s, 1H), 7.54 (s, 1H), 7.32 (d, J = 8.8 Hz, 1H), 6.78 (s, 1H), 6.26 (d, J = 16.2 Hz, 1H), 5.82 (s, 1H), 4.98 (s, 1H), 4.61 (d, J = 14.1 Hz, 1H), 4.54-4.29 (m, 5H), 4.21 (s, 4H), 3.83 (s, 2H), 3.48 (s, 1H), 3.13 (d, J = 1.7 Hz, 2H), 2.91-2.75 (m, 3H), 2.52 (s, 3H), 2.41 (s, 1H), 2.13- 2.03 (m, 1H), 1.83 (d, J = 7.5 Hz, 2H), 1.69 (dd, J = 12.4, 6.9 Hz, 1H). |
| 1-231 | 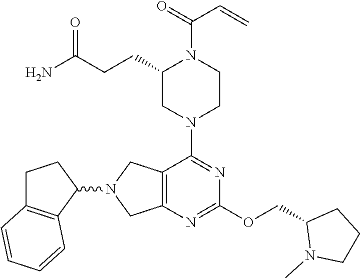 [SMILES: C=CC(=O)N1CCN(c2nc(OC[C@@H]3CCCN3C)nc3c2CN(C2CCc4ccccc42)C3)C[C@@H]1CCC(N)=O] | B | M + 1 = 560.3 | 0%/ 1% | 1H NMR (400 MHz, CD₃OD) δ 7.42 (d, J = 7.2 Hz, 1H), 7.23 (ddt, J = 14.0, 12.1, 6.9 Hz, 3H), 6.87- 6.70 (m, 1H), 6.25 (dd, J = 23.5, 17.2 Hz, 1H), 5.78 (d, J = 10.7 Hz, 1H), 4.82- 4.73 (m, 1H), 4.51 (ddd, J = 42.3, 30.4, 21.4 Hz, 3H), 4.35-4.19 (m, 5H), 4.00 (d, J = 13.0 Hz, 1H), 3.82 (s, 2H), 3.58-3.40 (m, 1H), 3.17-3.05 (m, 3H), 2.93-2.82 (m, 1H), 2.79-2.70 (m, 1H), 2.49 (s, 3H), 2.41-2.14 (m, 5H), 2.13-1.99 (m, 2H), 1.93-1.61 (m, 4H). |
| 1-232 | 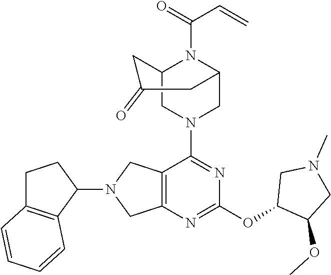 [SMILES: C=CC(=O)N1C2CC(=O)CC1CN(c1nc(O[C@@H]3CN(C)C[C@H]3OC)nc3c1CN(C1CCc4ccccc41)C3)C2] | A | M + 1 = 559.3 | 0%/ 0% | 1H NMR (400 MHz, CD₃OD) δ 7.43 (d, J = 7.1 Hz, 1H), 7.31-7.16 (m, 3H), 6.89 (dd, J = 16.7, 10.6 Hz, 0.5H), 6.68 (dd, J = 16.8, 10.7 Hz, 0.5H), 6.40-6.31 (m, 0.5H), 6.19 (dd, J = 16.8, 1.9 Hz, 0.5H), 5.93- 5.84 (m, 0.5H), 5.78 (dd, J = 10.7, 1.9 Hz, 0.5H), 5.54-5.40 (m, 1H), 5.22 (s, 1H), 4.64-4.01 (m, 7H), 3.93-3.68 (m, 3H), 3.59-3.38 (m, 7H), 3.20- 3.01 (m, 2H), 2.98-2.67 (m, 6H), 2.47-2.14 (m, 4H). |
| 1-233 | 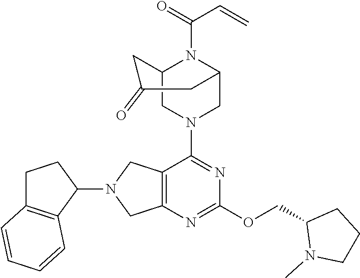 [SMILES: C=CC(=O)N1C2CC(=O)CC1CN(c1nc(OC[C@@H]3CCCN3C)nc3c1CN(C1CCc4ccccc41)C3)C2] | A | M + 1 = 543.3 | 0%/ 5% | 1H NMR (CH3CN-d3, 400 MHz): δ H 1.91-1.72 (4H, m), 2.35-2.04 (6H, m), 2.65-2.50 (2H, m), 2.90- 2.82 (1H, m), 3.20-2.98 (2H, m), 3.31-3.22 (1H, m), 3.78 (2H, s), 3.82 (1H, s), 4.11 (2H, s), 4.34-4.16 (4H, m), 4.57-4.39 (3H, m), 4.70 (1H, br.s), 4.87 (1H, br.s), 5.13 (1H, br.s), 5.70 (0.3H, d, J = 10.6 Hz), 5.80 (0.7H, d, J = 10.6 Hz), 6.13 (0.3H, d, J = 16.7 Hz), 6.29 (0.7H, d, J = 16.7 Hz), 6.59 (0.3H, dd, J = 16.8, 10.6 Hz), 6.80 (0.7H, dd, J = 16.7, 10.6 Hz), 7.31-7.21 (3H, m), 7.41 (1H, d, J = 7.1 Hz), 8.24 (1H, s). |
| 1-234 | 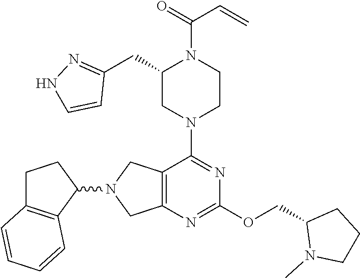 [SMILES: C=CC(=O)N1CCN(c2nc(OC[C@@H]3CCCN3C)nc3c2CN(C2CCc4ccccc42)C3)C[C@@H]1Cc1cc[nH]n1] | C | | | |
| 1-235 | 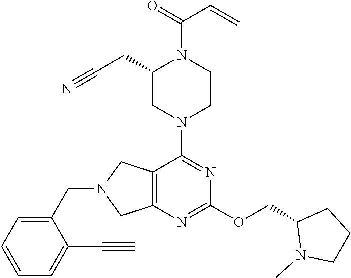 [SMILES: C#Cc1ccccc1CN1Cc2nc(OC[C@@H]3CCCN3C)nc(N3CCN(C(=O)C=C)[C@@H](CC#N)C3)c2C1] | A | M + 1 = 526.3 | 0%/ 0% | 1H NMR (400 MHz, CD₃OD) δ 8.46 (s, 2H), 7.86 (d, J = 7.6 Hz, 1H), 7.58 (ddd, J = 29.6, 18.8, 7.4 Hz, 3H), 6.75 (s, 1H), 6.33- 6.11 (m, 2H), 5.88 (d, J = 5.3 Hz, 2H), 5.52-5.20 (m, 5H), 5.03 (d, J = 15.4 Hz, 1H), 4.70 (s, 2H), 4.56 (dd, J = 12.2, 7.3 Hz, 1H), 4.18-3.77 (m, 2H), 3.58 (dd, J = 11.2, 6.3 Hz, 5H), 2.92 (d, J = 1.8 Hz, 6H), 2.36-2.26 (m, 1H), 2.08 (s, 3H). |
| 1-236 | 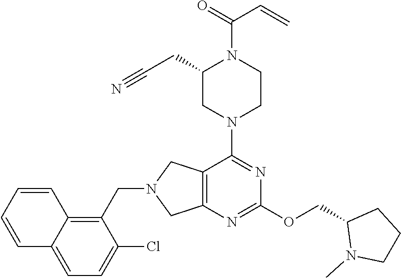 [SMILES: C=CC(=O)N1CCN(c2nc(OC[C@@H]3CCCN3C)nc3c2CN(Cc2c(Cl)ccc4ccccc24)C3)C[C@@H]1CC#N] | A | M + 1 = 586.2 | 94%/ 95% | 1H NMR (400 MHz, CD₃OD) δ 8.53 (s, 1H), 8.33 (d, J = 8.5 Hz, 1H), 7.88 (d, J = 7.6 Hz, 1H), 7.83 (d, J = 8.8 Hz, 1H), 7.60-7.55 (m, 1H), 7.54-7.48 (m, 2H), 6.76 (s, 1H), 6.27 (d, J = 16.6 Hz, 1H), 5.81 (d, J = 10.2 Hz, 1H), 4.96 (s, 1H), 4.78-4.42 (m, 5H), 4.36 (dd, J = 11.7, 6.8 Hz, 1H), 4.29-4.00 (m, 4H), 3.81 (s, 2H), 3.49 (d, J = 11.2 Hz, 1H), 3.15 (s, 3H), 2.96-2.76 (m, 2H), 2.68 (s, 4H), 2.22-2.13 (m, 1H), 1.90 (dd, J = 14.8, 8.0 Hz, 2H), 1.80 (dt, J = 12.6, 6.4 Hz, 1H). |
| 1-237 | 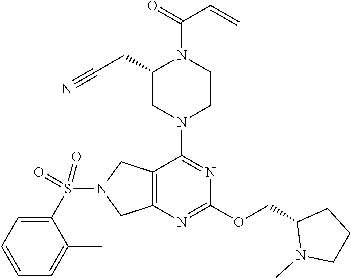 [SMILES: C=CC(=O)N1CCN(c2nc(OC[C@@H]3CCCN3C)nc3c2CN(S(=O)(=O)c2ccccc2C)C3)C[C@@H]1CC#N] | A | M + 1 = 566.3 | 27%/ 80% | 1H NMR (400 MHz, CD₃OD) δ 7.90 (d, J = 7.5 Hz, 1H), 7.54 (td, J = 7.5, 1.2 Hz, 1H), 7.47-7.34 (m, 2H), 6.77 (s, 1H), 6.27 (d, J = 16.4 Hz, 1H), 5.82 (d, J = 10.1 Hz, 1H), 4.97 (s, 1H), 4.85 (s, 2H), 4.66 (d, J = 14.0 Hz, 2H), 4.33 (dd, J = 16.6, 10.4 Hz, 4H), 4.10 (d, J = 12.7 Hz, 1H), 3.49 (d, J = 64.2 Hz, 3H), 3.13-3.06 (m, 1H), 2.98- 2.73 (m, 3H), 2.65 (s, 3H), 2.50 (d, J = 8.7 Hz, 3H), 2.39 (dd, J = 18.1, 9.2 Hz, 1H), 2.08 (ddd, J = 16.3, 12.5, 8.3 Hz, 1H), 1.87- 1.76 (m, 2H), 1.75-1.63 (m, 1H). |
| 1-238 | 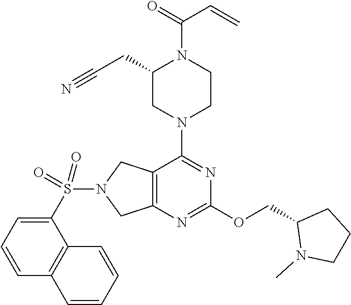 [SMILES: C=CC(=O)N1CCN(c2nc(OC[C@@H]3CCCN3C)nc3c2CN(S(=O)(=O)c2cccc4ccccc24)C3)C[C@@H]1CC#N] | A | M + 1 = 602.3 | 73%/ 95% | 1H NMR (400 MHz, CD₃OD) δ 8.83 (d, J = 8.6 Hz, 1H), 8.22 (dd, J = 19.8, 7.8 Hz, 2H), 8.02 (d, J = 8.1 Hz, 1H), 7.75-7.60 (m, 3H), 6.77 (s, 1H), 6.27 (d, J = 16.5 Hz, 1H), 5.82 (d, J = 10.4 Hz, 1H), 4.92 (s, 2H), 4.60 (d, J = 13.6 Hz, 1H), 4.31 (dd, J = 35.4, 28.9 Hz, 5H), 4.06 (s, 2H), 3.41 (d, J = 48.9 Hz, 2H), 3.13-3.08 (m, 1H), 2.84 (dd, J = 16.5, 7.7 Hz, 3H), 2.50 (s, 3H), 2.45-2.36 (m, 1H), 2.11-2.01 (m, 1H), 1.93-1.58 (m, 4H). |
| 1-239 | 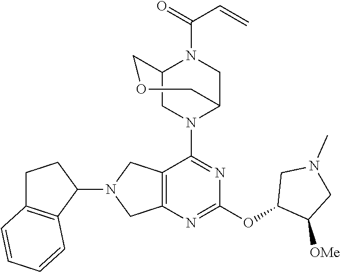 [SMILES: C=CC(=O)N1CC2COCC1CN2c1nc(O[C@@H]2CN(C)C[C@H]2OC)nc2c1CN(C1CCc3ccccc31)C2] | | | | |
| 1-240 | 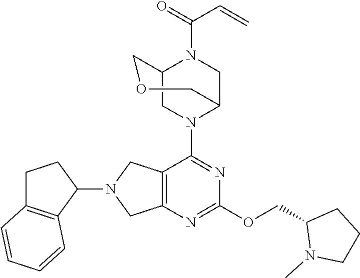 [SMILES: C=CC(=O)N1CC2COCC1CN2c1nc(OC[C@@H]2CCCN2C)nc2c1CN(C1CCc3ccccc31)C2] | | | | |
| 1-241 | 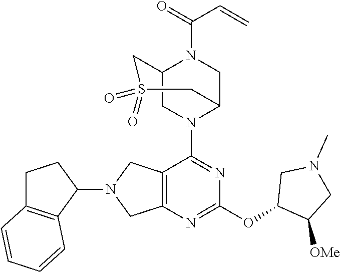 [SMILES: C=CC(=O)N1CC2CS(=O)(=O)CC1CN2c1nc(O[C@@H]2CN(C)C[C@H]2OC)nc2c1CN(C1CCc3ccccc31)C2] | | | | |
| 1-242 | 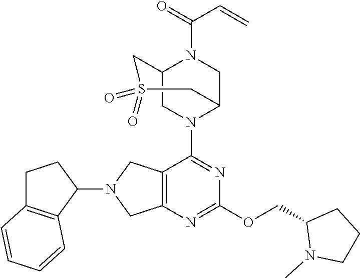 [SMILES: C=CC(=O)N1CC2CS(=O)(=O)CC1CN2c1nc(OC[C@@H]2CCCN2C)nc2c1CN(C1CCc3ccccc31)C2] | | | | |
| 1-243 | 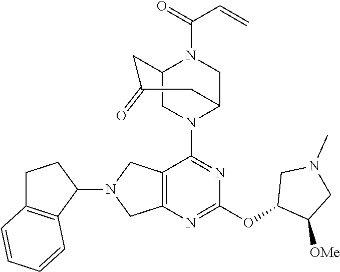 [SMILES: C=CC(=O)N1CC2CC(=O)CC1CN2c1nc(O[C@@H]2CN(C)C[C@H]2OC)nc2c1CN(C1CCc3ccccc31)C2] | | | | |
| 1-244 | 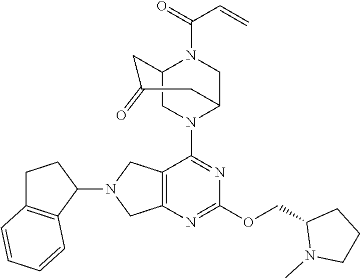 [SMILES: C=CC(=O)N1CC2CC(=O)CC1CN2c1nc(OC[C@@H]2CCCN2C)nc2c1CN(C1CCc3ccccc31)C2] | | | | |
| 1-245 | 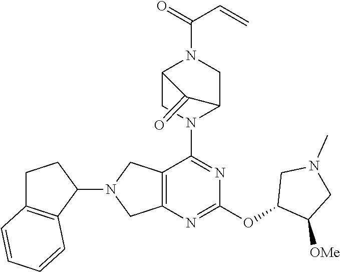 [SMILES: C=CC(=O)N1CC2C(=O)C1CN2c1nc(O[C@@H]2CN(C)C[C@H]2OC)nc2c1CN(C1CCc3ccccc31)C2] | | | | |
| 1-246 | 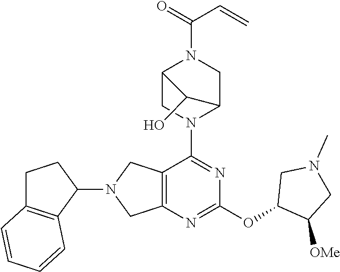 [SMILES: C=CC(=O)N1CC2C(O)C1CN2c1nc(O[C@@H]2CN(C)C[C@H]2OC)nc2c1CN(C1CCc3ccccc31)C2] | | | | |
| 1-247 | 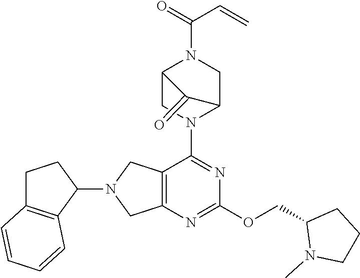 [SMILES: C=CC(=O)N1CC2C(=O)C1CN2c1nc(OC[C@@H]2CCCN2C)nc2c1CN(C1CCc3ccccc31)C2] | | | | |
| 1-248 | 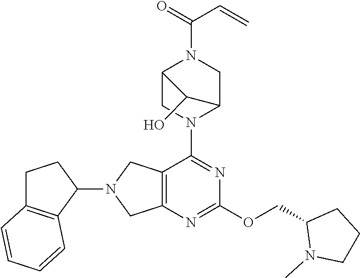 [SMILES: C=CC(=O)N1CC2C(O)C1CN2c1nc(OC[C@@H]2CCCN2C)nc2c1CN(C1CCc3ccccc31)C2] | | | | |
| 1-249 | 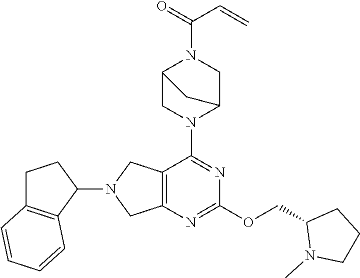 [SMILES: C=CC(=O)N1CC2CC1CN2c1nc(OC[C@@H]2CCCN2C)nc2c1CN(C1CCc3ccccc31)C2] | A | M + 1 = 501.3 | 0%/ 0% | ^{l}H NMR (CD₃CN, 400 MHz) δ 1.95-1.81 (4H, m), 2.19 (3H, m), 2.76 (3H, s), 2.85-2.80 (2H, m), 3.07-2.99 (1H, m), 3.52-3.42 (3H, m), 3.66- 3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-250 | 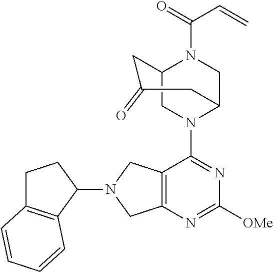 [SMILES: C=CC(=O)N1CC2CC(=O)CC1CN2c1nc(OC)nc2c1CN(C1CCc3ccccc31)C2] | A | M + 1 = 460.2 | 0%/ 0% | ^{l}H NMR (CD₃CN, 400 MHz) δ 2.27 (4H, m), 2.65-2.54 (2H, m), 2.87-2.79 (1H, m), 3.13-2.98 (3H, m), 3.77 (2H, m), 3.83-3.81 (3H, m), 4.29-4.02 (3H, m), 4.50 (1H, t, J = 6.2 Hz), 4.66 (1H, br.s), 4.84 (1H, br.s), 5.10 (1H, br.s), 5.77 (1H, dd, J = 10.5, 2.1 Hz), 6.26 (1H, dd, J = 16.8, 2.1 Hz), 6.77 (1H, dd, J = 16.8, 10.6 Hz), 7.28-7.18 (3H, m), 7.39 (1H, d, J = 7.1 Hz). |
| 1-251 | 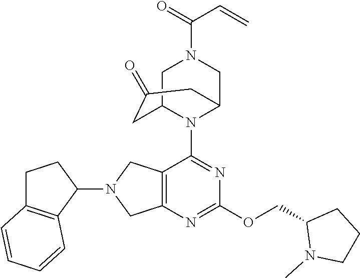 [SMILES: C=CC(=O)N1CC2CC(=O)CC(C1)N2c1nc(OC[C@@H]2CCCN2C)nc2c1CN(C1CCc3ccccc31)C2] | A | M + 1 = 543.3 | 0%/ 0% | ^{l}H NMR (CD₃CN, 400 MHz) δ 2.00-1.75 (6H, m), 2.34-2.10 (4H, m), 2.70-2.59 (6H, m), 2.90- 2.78 (1H, m), 3.10-3.00 (1H, m), 3.21-3.12 (1H, m), 3.37-3.29 (2H, m), 3.83 (2H, br.s), 4.19 (2H, br.s), 4.38 (1H, dd, J = 11.5, 4.9 Hz), 4.56-4.50 (3H, m), 4.87 (2H, br.s), 5.70 (1H, dd, J = 10.5, 2.2 Hz), 6.13 (1H, dd, J = 16.7, 2.2 Hz), 6.59 (1H, dd, J = 16.7, 10.6 Hz), 7.30-7.21 (3H, m), 7.41 (1H, d, J = 7.0 Hz). |
| 1-252 | 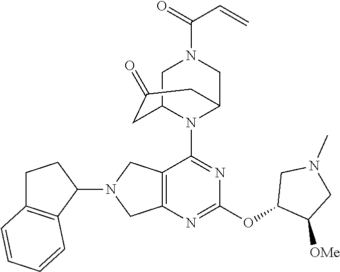 [SMILES: C=CC(=O)N1CC2CC(=O)CC(C1)N2c1nc(O[C@@H]2CN(C)C[C@H]2OC)nc2c1CN(C1CCc3ccccc31)C2] | A | M + 1 = 559.4 | 0%/ 0% | ^{l}H NMR (400 MHz, CD₃OD) δ 7.43 (d, J = 7.2 Hz, 1H), 7.33-7.17 (m, 3H), 6.70 (dd, J = 16.8, 10.7 Hz, 1H), 6.19 (dd, J = 16.8, 1.9 Hz, 1H), 5.77 (dd, J = 10.7, 1.9 Hz, 1H), 5.63 (d, J = 5.1 Hz, 1H), 5.33 (s, 2H), 4.59 (dd, J = 7.4, 4.5 Hz, 2H), 4.27-4.10 (m, 2H), 4.10-3.92 (m, 4H), 3.70 (dd, J = 12.5, 5.2 Hz, 1H), 3.57-3.47 (m, 1H), 3.46 (d, J = 1.3 Hz, 3H), 3.40 (d, J = 12.9 Hz, 1H), 3.16-2.86 (m, 4H), 2.83 (s, 3H), 2.65 (bs, 2H), 2.43-2.09 (m, 5H). |
| 1-253 | 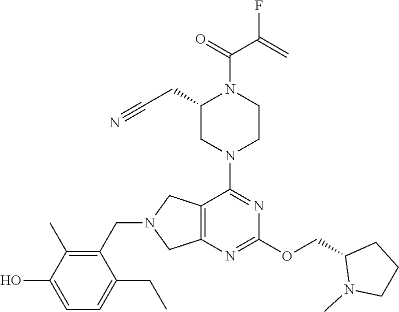 [SMILES: C=C(F)C(=O)N1CCN(c2nc(OC[C@@H]3CCCN3C)nc3c2CN(Cc2c(CC)ccc(O)c2C)C3)C[C@@H]1CC#N] | A | M + 1 = 578.3 | 93%/ 94% | 1H NMR (400 MHz, CD₃OD) δ 8.37 (s, 4H), 6.87 (d, J = 8.3 Hz, 1H), 6.68 (d, J = 8.2 Hz, 1H), 5.31 (dt, J = 13.5, 12.5 Hz, 2H), 4.76 (d, J = 12.5 Hz, 2H), 4.48 (s, 1H), 4.13 (s, 3H), 3.97 (s, 2H), 3.81 (s, 1H), 3.68 (d, J = 17.8 Hz, 3H), 3.36 (d, J = 13.9 Hz, 1H), 3.25- 3.09 (m, 2H), 3.01 (s, 3H), 2.90 (dd, J = 27.5, 7.7 Hz, 2H), 2.81-2.65 (m, 4H), 2.39-2.31 (m, 1H), 2.27 (s, 3H), 2.25-1.86 (m, 4H), 1.15 (t, J = 7.3 Hz, 3H). |
| 1-254 | 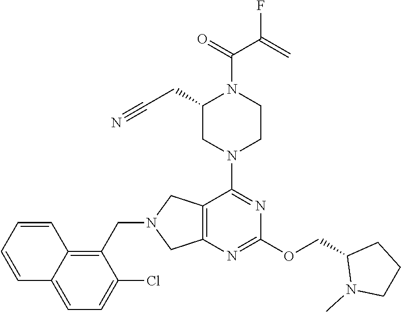 [SMILES: C=C(F)C(=O)N1CCN(c2nc(OC[C@@H]3CCCN3C)nc3c2CN(Cc2c(Cl)ccc4ccccc24)C3)C[C@@H]1CC#N] | A | M + 1 = 604.25 | 31%/ 73% | 1H NMR (400 MHz, CD₃OD) δ = 8.51 (s, 1H), 8.34 (d, J = 8.5, 1H), 7.89 (d, J = 8.7, 1H), 7.84 (d, J = 8.8, 1H), 7.61-7.49 (m, 3H), 5.31 (t, J = 20.0, 2H), 4.92 (s, 1H), 4.85-4.55 (m, 5H), 4.48-4.36 (m, 1H), 4.35-3.91 (m, 4H), 3.81 (s, 2H), 3.40 (d, J = 11.0, 3H), 3.28 (s, 1H), 2.99-2.84 (m, 3H), 2.83 (s, 3H), 2.35-2.15 (m, 1H), 2.11- 1.92 (m, 2H), 1.86 (t, J = 15.0, 1H). |
| 1-255 | 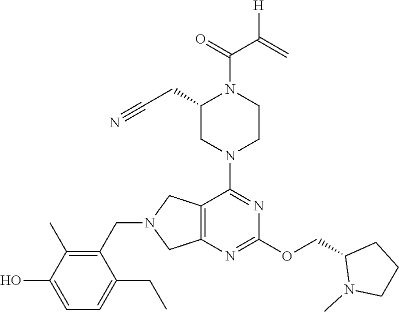 [SMILES: C=CC(=O)N1CCN(c2nc(OC[C@@H]3CCCN3C)nc3c2CN(Cc2c(CC)ccc(O)c2C)C3)C[C@@H]1CC#N] | A | M + 1 = 560.3 | 92%/ 94% | 1H NMR (400 MHz, CD₃OD) δ 8.34 (s, 2H), 6.88 (d, J = 8.3 Hz, 1H), 6.73 (d, J = 24.3 Hz, 1H), 6.68 (d, J = 8.3 Hz, 1H), 6.27 (d, J = 16.8 Hz, 1H), 5.82 (d, J = 9.9 Hz, 1H), 4.93 (s, 1H), 4.83-4.74 (m, 2H), 4.47 (dd, J = 12.6, 7.4 Hz, 1H), 4.15 (s, 3H), 4.08 (s, 1H), 3.98 (s, 2H), 3.84 (d, J = 5.2 Hz, 1H), 3.75-3.61 (m, 3H), 3.43 (dd, J = 55.6, 8.3 Hz, 2H), 3.19 (ddd, J = 52.4, 27.9, 15.5 Hz, 2H), 3.02 (s, 3H), 2.89 (s, 1H), 2.72 (q, J = 7.5 Hz, 3H), 2.43-2.30 (m, 1H), 2.27 (s, 3H), 2.22-1.93 (m, 4H), 1.15 (t, J = 7.5 Hz, 3H). |
| 1-301 | 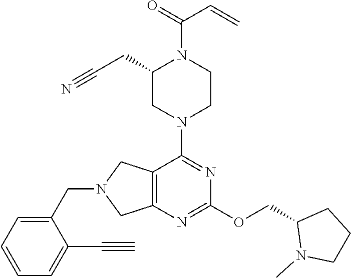 [SMILES: C#Cc1ccccc1CN1Cc2nc(OC[C@@H]3CCCN3C)nc(N3CCN(C(=O)C=C)[C@@H](CC#N)C3)c2C1] | B | M + 1 = 526.3 | 0%/ 0% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21- 4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-303 | 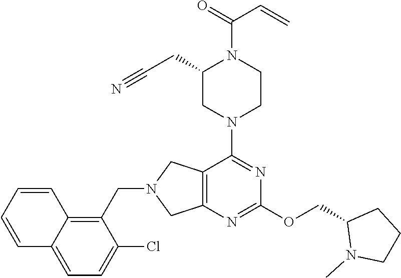 [SMILES: C=CC(=O)N1CCN(c2nc(OC[C@@H]3CCCN3C)nc3c2CN(Cc2c(Cl)ccc4ccccc24)C3)C[C@@H]1CC#N] | B | M + 1 = 586.2 | 94%/ 95% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-304 | 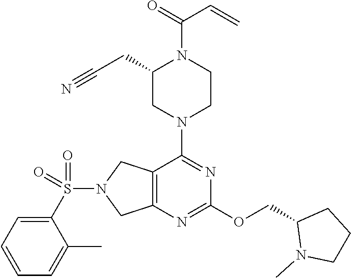 [SMILES: C=CC(=O)N1CCN(c2nc(OC[C@@H]3CCCN3C)nc3c2CN(S(=O)(=O)c2ccccc2C)C3)C[C@@H]1CC#N] | R | M + 1 = 566.3 | 27%/ 80% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29- 7.18 (3H, m), 7.39 (1H, d, J = 7.2 Hz). |
| 1-305 | 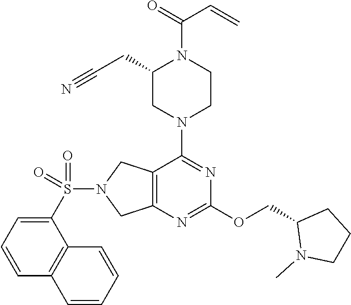 [SMILES: C=CC(=O)N1CCN(c2nc(OC[C@@H]3CCCN3C)nc3c2CN(S(=O)(=O)c2cccc4ccccc24)C3)C[C@@H]1CC#N] | R | M + 1 = 602.3 | 73%/ 95% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29- 7.18 (3H, m), 7.39 (1H, d, J = 7.2 Hz). |
| 1-307 | 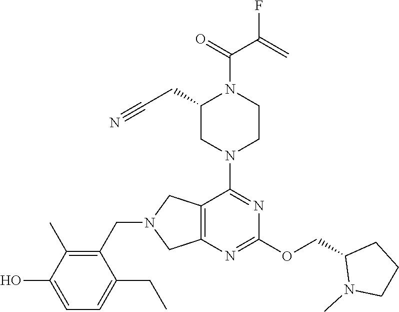 [SMILES: C=C(F)C(=O)N1CCN(c2nc(OC[C@@H]3CCCN3C)nc3c2CN(Cc2c(CC)ccc(O)c2C)C3)C[C@@H]1CC#N] | B | M + 1 = 578.3 | 93%/ 94% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s). 4.89 (1H, br.s). 5.66 (1H, m), 6.21 (1H, d, J = 16.8 Hz), 6.37 (1H, dd, J = 18.4. 10.6 Hz), 6.67-6.59 (1H, m), 7.29- 7.18 (3H, m), 7.39 (1H, d, J = 7.2 Hz). |
| 1-308 | 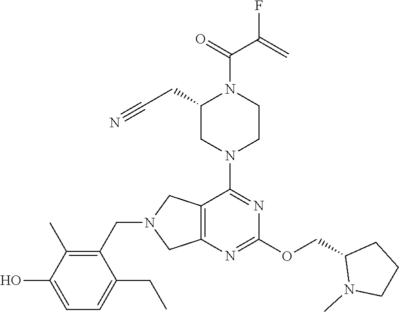 | B | M + 1 = 604.25 | 31%/ 73% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4. 10.6 Hz), 6.67-6.59 (1H, m), 7.29-7.18 (3H, m), 7.39 (1H, d, J = 7.2 Hz). |
| 1-309 | 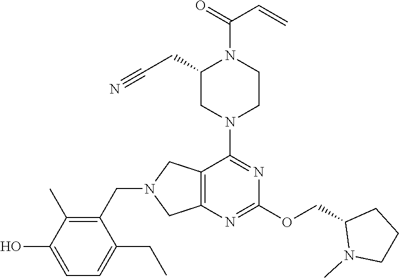 [SMILES: C=CC(=O)N1CCN(c2nc(OC[C@@H]3CCCN3C)nc3c2CN(Cc2c(CC)ccc(O)c2C)C3)C[C@@H]1CC#N] | B | M + 1 = 560.3 | 92%/ 94% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52-3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-311 | 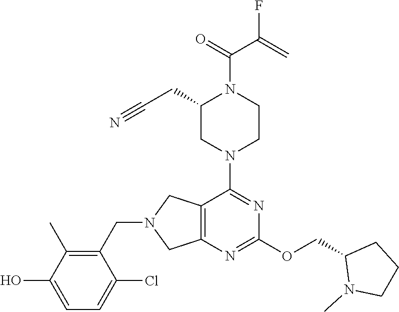 [SMILES: C=C(F)C(=O)N1CCN(c2nc(OC[C@@H]3CCCN3C)nc3c2CN(Cc2c(Cl)ccc(O)c2C)C3)C[C@@H]1CC#N] | B | M + 1 = 584.2 | 93%/ 95% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29- 7.18 (3H, m), 7.39 (1H, d, J = 7.2 Hz). |
| 1-312 | 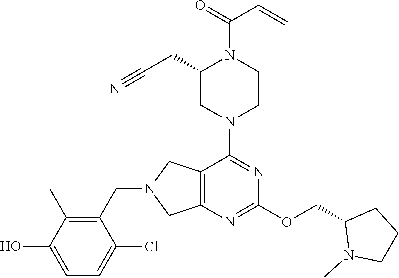 [SMILES: C=CC(=O)N1CCN(c2nc(OC[C@@H]3CCCN3C)nc3c2CN(Cc2c(Cl)ccc(O)c2C)C3)C[C@@H]1CC#N] | B | M + 1 = 566.2 | 95%/ 95% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4. 10.6 Hz), 6.67-6.59 (1H, m), 7.29- 7.18 (3H, m), 7.39 (1H, d, J = 7.2 Hz). |
| 1-313 | 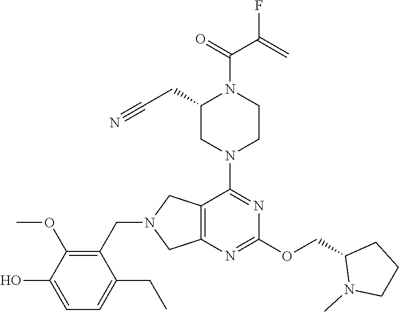 [SMILES: C=C(F)C(=O)N1CCN(c2nc(OC[C@@H]3CCCN3C)nc3c2CN(Cc2c(CC)ccc(O)c2OC)C3)C[C@@H]1CC#N] | B | M + 1 = 594.5 | 87%/ 95% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s). 4.89 (1H, br.s), 5.66 (1H, m), 6.21 (1H, d, J = 16.8 Hz), 6.37 (1H, dd, J = 18.4. 10.6 Hz), 6.67-6.59 (1H, m), 7.29- 7.18 (3H, m), 7.39 (1H, d, J = 7.2 Hz). |
| 1-314 | 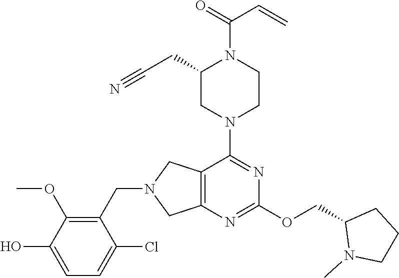 | B | M + 1 = 576.4 | 68%/ 68% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4. 10.6 Hz), 6.67-6.59 (1H, m), 7.29-7.18 (3H, m), 7.39 (1H, d, J = 7.2 Hz). |
| 1-315 | 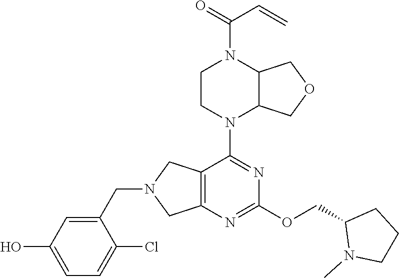 [SMILES: C=CC(=O)N1CCN(c2nc(OC[C@@H]3CCCN3C)nc3c2CN(Cc2cc(O)ccc2Cl)C3)C2COCC21] | A | M + 1 = 555.3 | 41%/ 66% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29- 7.18 (3H, m), 7.39 (1H, d, J = 7.2 Hz). |
| 1-316 | 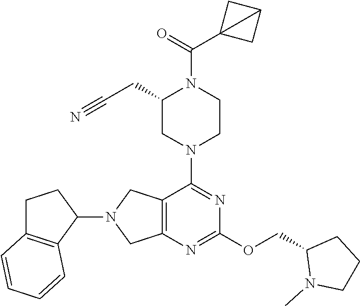 [SMILES: CN1CCC[C@H]1COc1nc2c(c(N3CCN(C(=O)C45CC4C5)[C@@H](CC#N)C3)n1)CN(C1CCc3ccccc31)C2] | B | M + 1 = 554.3 | 0%/ 0% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4. 10.6 Hz), 6.67-6.59 (1H, m), 7.29-7.18 (3H, m), 7.39 (1H, d, J = 7.2 Hz). |
| 1-317 | 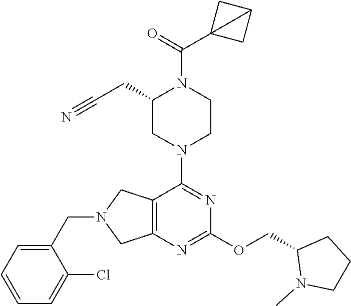 [SMILES: CN1CCC[C@H]1COc1nc2c(c(N3CCN(C(=O)C45CC4C5)[C@@H](CC#N)C3)n1)CN(Cc1ccccc1Cl)C2] | B | M + 1 = 562.2 | 0%/ 0% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-318 | 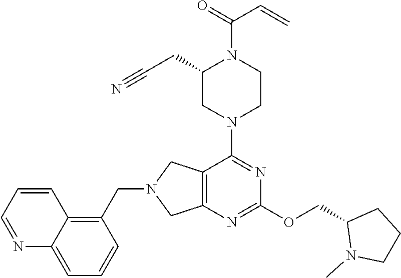 [SMILES: C=CC(=O)N1CCN(c2nc(OC[C@@H]3CCCN3C)nc3c2CN(Cc2cccc4ncccc24)C3)C[C@@H]1CC#N] | I | M + 1 = 553.3 | 1%/ 11% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29- 7.18 (3H, m), 7.39 (1H, d, J = 7.2 Hz). |
| 1-319 | 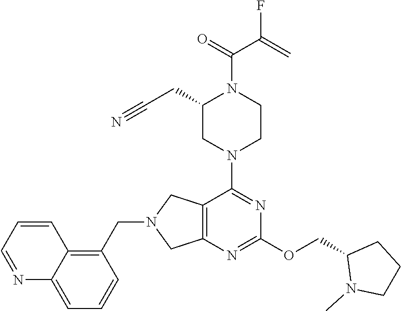 [SMILES: C=C(F)C(=O)N1CCN(c2nc(OC[C@@H]3CCCN3C)nc3c2CN(Cc2cccc4ncccc24)C3)C[C@@H]1CC#N] | I | M + 1 = 571.3 | 0%/ 0% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-320 | 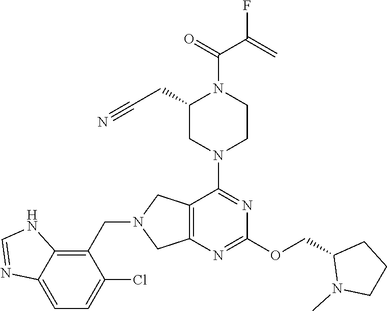 [SMILES: C=C(F)C(=O)N1CCN(c2nc(OC[C@@H]3CCCN3C)nc3c2CN(Cc2c(Cl)ccc4nc[nH]c24)C3)C[C@@H]1CC#N] | I | M + 1 = 594.2 | 76%/ 91% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-321 | 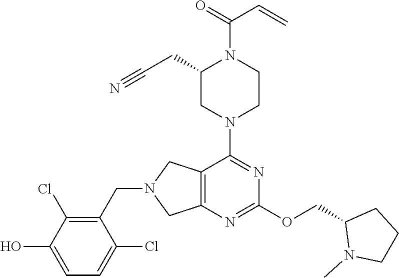 [SMILES: C=CC(=O)N1CCN(c2nc(OC[C@@H]3CCCN3C)nc3c2CN(Cc2c(Cl)ccc(O)c2Cl)C3)C[C@@H]1CC#N] | B | M + 1 = 586.2 | 88%/ 90% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-322 | 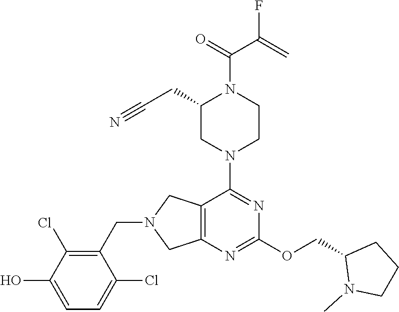 [SMILES: C=C(F)C(=O)N1CCN(c2nc(OC[C@@H]3CCCN3C)nc3c2CN(Cc2c(Cl)ccc(O)c2Cl)C3)C[C@@H]1CC#N] | B | M + 1 = 604.2 | 76%/ 94% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52-3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4. 10.6 Hz), 6.67-6.59 (1H, m), 7.29-7.18 (3H, m), 7.39 (1H, d, J = 7.2 Hz). |
| 1-323 | 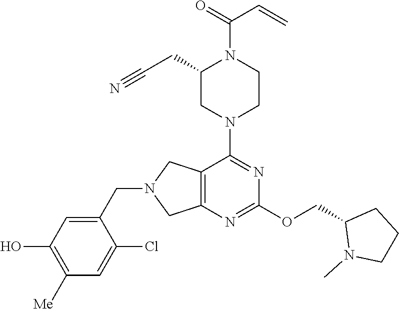 [SMILES: C=CC(=O)N1CCN(c2nc(OC[C@@H]3CCCN3C)nc3c2CN(Cc2cc(O)c(C)cc2Cl)C3)C[C@@H]1CC#N] | B | M + 1 = 566.2 | 72%/ 93% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-324 | 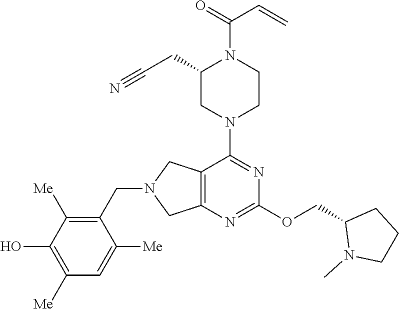 [SMILES: C=CC(=O)N1CCN(c2nc(OC[C@@H]3CCCN3C)nc3c2CN(Cc2c(C)cc(C)c(O)c2C)C3)C[C@@H]1CC#N] | B | M + 1 = 560.3 | 31%/ 78% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52-3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-325 | 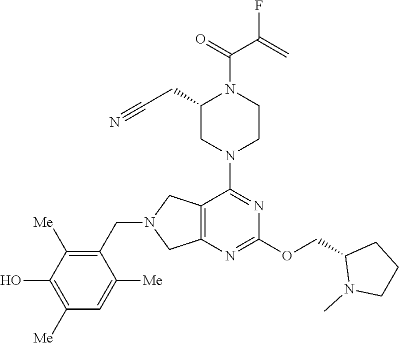 [SMILES: C=C(F)C(=O)N1CCN(c2nc(OC[C@@H]3CCCN3C)nc3c2CN(Cc2c(C)cc(C)c(O)c2C)C3)C[C@@H]1CC#N] | B | M + 1 = 578.3 | 0%/ 3% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52-3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-326 | 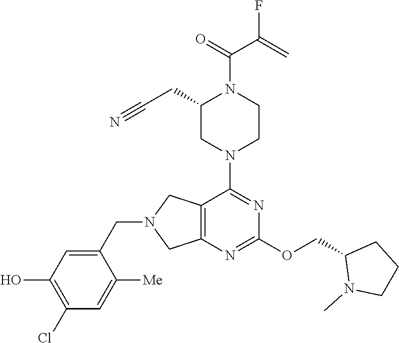 [SMILES: C=C(F)C(=O)N1CCN(c2nc(OC[C@@H]3CCCN3C)nc3c2CN(Cc2cc(O)c(Cl)cc2C)C3)C[C@@H]1CC#N] | S | M + 1 = 584.2 | 0%/ 2% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4. 10.6 Hz), 6.67-6.59 (1H, m), 7.29-7.18 (3H, m), 7.39 (1H, d, J = 7.2 Hz). |
| 1-327 | 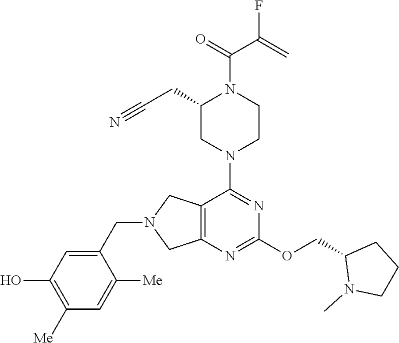 [SMILES: C=C(F)C(=O)N1CCN(c2nc(OC[C@@H]3CCCN3C)nc3c2CN(Cc2cc(O)c(C)cc2C)C3)C[C@@H]1CC#N] | B | M + 1 = 564.3 | 0%/ 3% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-328 | 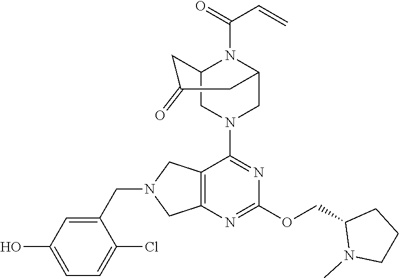 [SMILES: C=CC(=O)N1C2CC(=O)CC1CN(c1nc(OC[C@@H]3CCCN3C)nc3c1CN(Cc1cc(O)ccc1Cl)C3)C2] | B | M + 1 = 567.3 | 14%/ 39% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-329 | 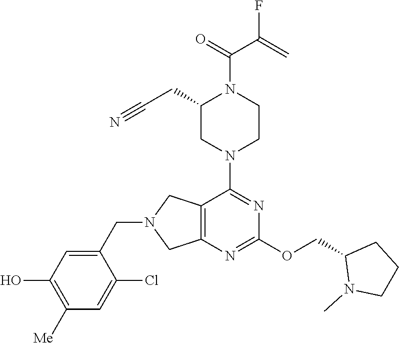 [SMILES: C=C(F)C(=O)N1CCN(c2nc(OC[C@@H]3CCCN3C)nc3c2CN(Cc2cc(O)c(C)cc2Cl)C3)C[C@@H]1CC#N] | B | M + 1 = 584.0 | 2%/ 22% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-330 | 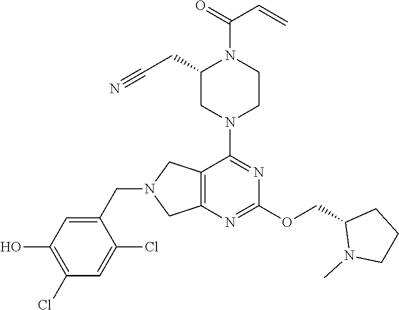 [SMILES: C=CC(=O)N1CCN(c2nc(OC[C@@H]3CCCN3C)nc3c2CN(Cc2cc(O)c(Cl)cc2Cl)C3)C[C@@H]1CC#N] | I | M + 1 = 586.0 | 52%/ 86% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4. 10.6 Hz), 6.67-6.59 (1H, m), 7.29-7.18 (3H, m), 7.39 (1H, d, J = 7.2 Hz). |
| 1-331 | 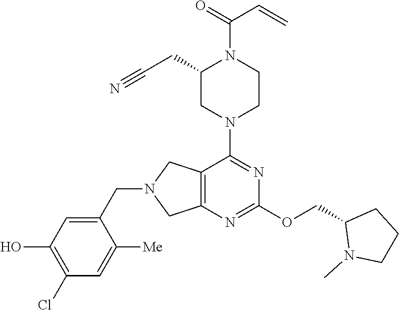 [SMILES: C=CC(=O)N1CCN(c2nc(OC[C@@H]3CCCN3C)nc3c2CN(Cc2cc(O)c(Cl)cc2C)C3)C[C@@H]1CC#N] | B | M + 1 = 566.2 | 32%/ 76% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52-3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-332 | 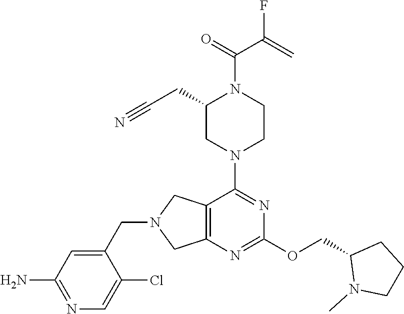 [SMILES: C=C(F)C(=O)N1CCN(c2nc(OC[C@@H]3CCCN3C)nc3c2CN(Cc2cc(N)ncc2Cl)C3)C[C@@H]1CC#N] | S | M + 1 = 570.4 | 6%/ 48% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52-3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-333 | 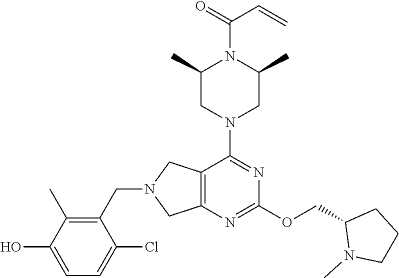 [SMILES: C=CC(=O)N1[C@H](C)CN(c2nc(OC[C@@H]3CCCN3C)nc3c2CN(Cc2c(Cl)ccc(O)c2C)C3)C[C@@H]1C] | A | M + 1 = 555.3 | 17%/ 59% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-334 | 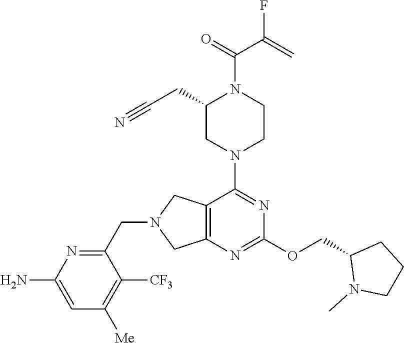 [SMILES: C=C(F)C(=O)N1CCN(c2nc(OC[C@@H]3CCCN3C)nc3c2CN(Cc2nc(N)cc(C)c2C(F)(F)F)C3)C[C@@H]1CC#N] | S | M + 1 = 618.3 | 79%/ 86% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-335 | 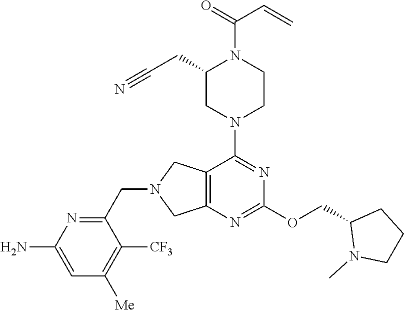 [SMILES: C=CC(=O)N1CCN(c2nc(OC[C@@H]3CCCN3C)nc3c2CN(Cc2nc(N)cc(C)c2C(F)(F)F)C3)C[C@@H]1CC#N] | B | M + 1 = 600.3 | 92%/ 95% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-336 | 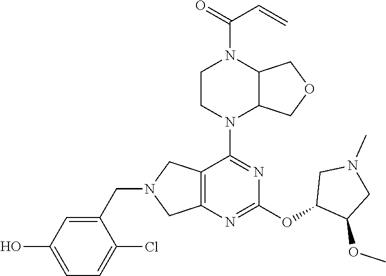 [SMILES: C=CC(=O)N1CCN(c2nc(O[C@@H]3CN(C)C[C@H]3OC)nc3c2CN(Cc2cc(O)ccc2Cl)C3)C2COCC21] | A | M + 1 = 571.3 | 0%/ 9% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-337 | 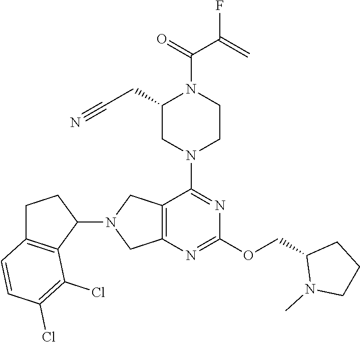 [SMILES: C=C(F)C(=O)N1CCN(c2nc(OC[C@@H]3CCCN3C)nc3c2CN(C2CCc4ccc(Cl)c(Cl)c42)C3)C[C@@H]1CC#N] | I | M + 1 = 614.2 | 27%/ 74% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-338 | 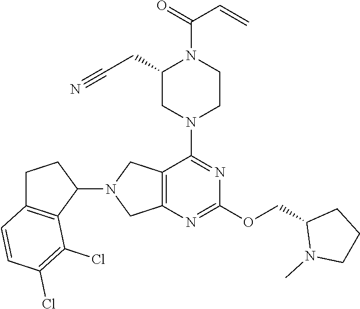 [SMILES: C=CC(=O)N1CCN(c2nc(OC[C@@H]3CCCN3C)nc3c2CN(C2CCc4ccc(Cl)c(Cl)c42)C3)C[C@@H]1CC#N] | I | M + 1 = 578.3 | 93%/ 95% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52-3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29- 7.18 (3H, m), 7.39 (1H, d, J = 7.2 Hz). |
| 1-339 | 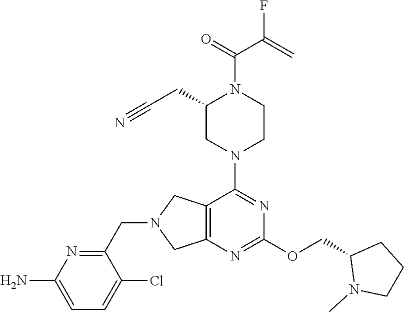 [SMILES: C=C(F)C(=O)N1CCN(c2nc(OC[C@@H]3CCCN3C)nc3c2CN(Cc2nc(N)ccc2Cl)C3)C[C@@H]1CC#N] | S | M + 1 = 570.3 | 83%/ 92% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52-3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29- 7.18 (3H, m), 7.39 (1H, d, J = 7.2 Hz). |
| 1-340 | 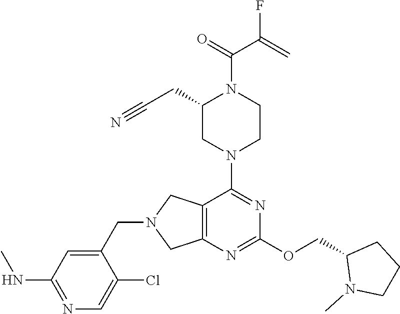 [SMILES: C=C(F)C(=O)N1CCN(c2nc(OC[C@@H]3CCCN3C)nc3c2CN(Cc2cc(NC)ncc2Cl)C3)C[C@@H]1CC#N] | S | M + 1 = 584.4 | 0%/ 7% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52-3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4. 10.6 Hz), 6.67-6.59 (1H, m), 7.29-7.18 (3H, m), 7.39 (1H, d, J = 7.2 Hz). |
| 1-341 | 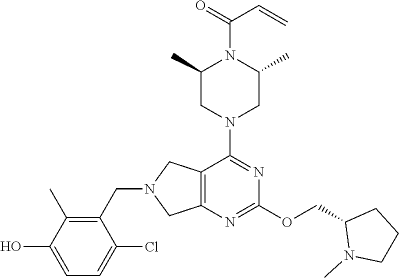 [SMILES: C=CC(=O)N1[C@H](C)CN(c2nc(OC[C@@H]3CCCN3C)nc3c2CN(Cc2c(Cl)ccc(O)c2C)C3)C[C@H]1C] | A | M + 1 = 555.3 | 0%/ 0% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-342 | 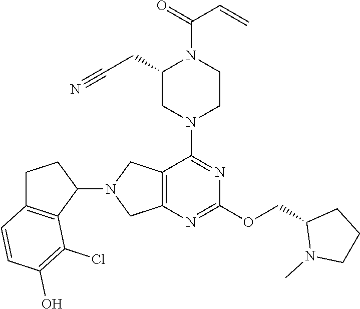 [SMILES: C=CC(=O)N1CCN(c2nc(OC[C@@H]3CCCN3C)nc3c2CN(C2CCc4ccc(O)c(Cl)c42)C3)C[C@@H]1CC#N] | I | M + 1 = 578.3 | 80%/ 87% | 1H NMR (CH3CN-d3, 400 MHz): dH 1.95-1.81 (4H, m), 2.19 (3H, m), 2.76 (3H, s), 2.85-2.80 (2H, m), 3.07-2.99 (1H, m), 3.52- 3.42 (3H, m), 3.66-3.57 (3H, m), 3.79 (2H, s), 4.21-4.09 (2H, m), 4.49 (3H, m), 4.77 (1H, br.s), 4.89 (1H, br.s), 5.66 (1H, m), 6.21 (1H, d, J = 16.8 Hz), 6.37 (1H, dd, J = 18.4, 10.6 Hz), 6.67-6.59 (1H, m), 7.29-7.18 (3H, m), 7.39 (1H, d, J = 7.2 Hz). |
| 1-343 | 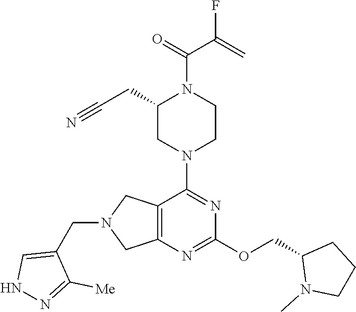 [SMILES: C=C(F)C(=O)N1CCN(c2nc(OC[C@@H]3CCCN3C)nc3c2CN(Cc2c[nH]nc2C)C3)C[C@@H]1CC#N] | S | M + 1 = 524.3 | 0%/ 0% | 1H NMR (CD3CN, 400 MHz) δ 1.81-1.63 (3H, m), 2.02-1.98 (1H, m), 2.28- 2.20 (5H, m), 2.39 (3H, s), 2.58-2.53 (1H, m), 2.77 (1H, dd, J = 17.1, 6.7 Hz), 2.91-2.85 (1H, m), 3.02- 2.98 (1H, m), 3.17-3.07 (1H, m), 3.24 (1H, d, J = 12 Hz), 3.42 (1H, br. s), 3.68- 3.64 (2H, m), 3.72 (2H, s), 4.02-3.94 (2H, m), 4.14- 4.10 (2H, m), 4.29 (1H, dd, J = 10.8, 5.1 Hz), 4.43 (1H, d, J = 14.0 Hz), 4.81 (1H, br. s), 5.28-5.15 (2H, m), 7.43 (1H, s). |
| 1-344 | 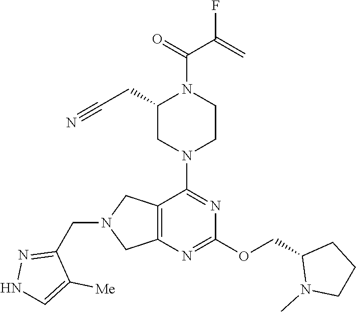 [SMILES: C=C(F)C(=O)N1CCN(c2nc(OC[C@@H]3CCCN3C)nc3c2CN(Cc2n[nH]cc2C)C3)C[C@@H]1CC#N] | S | M + 1 = 524.3 | 0%/ 2% | 1H NMR (CD3OD, 400 MHz) δ 2.20-1.92 (7H, m), 2.39-2.27 (1H, m), 3.05- 2.82 (6H, m), 3.24-3.12 (1H, m), 3.42-3.31 (2H, m), 3.70-3.60 (1H, m), 3.84-3.73 (3H, m), 3.93 (2H, s), 4.20- 4.10 (4H, m), 4.47 (1H, dd, J = 12.5, 7.3 Hz), 4.75 (2H, d, J = 13.4 Hz), 5.38- 5.26 (2H, m), 7.39 (1H, s). |
| 1-345 | 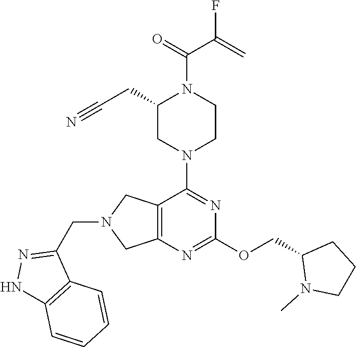 [SMILES: C=C(F)C(=O)N1CCN(c2nc(OC[C@@H]3CCCN3C)nc3c2CN(Cc2n[nH]c4ccccc24)C3)C[C@@H]1CC#N] | S | M + 1 = 560.3 | 0%/ 0% | 1H NMR (CD3CN, 400 MHz) δ 1.80-1.71 (1H, m), 1.92-1.82 (2H, m), 2.16-2.05 (1H, m), 2.59-2.52 (4H, m), 2.88-2.72 (2H, m), 3.15- 3.03 (2H, m), 3.57-3.22 (3H, m), 3.80 (2H, s), 4.16- 3.90 (4H, m), 4.25 (2H, s), 4.32 (1H, dd, J = 11.5, 5.1 Hz), 4.44-4.40 (2H, m), 4.79 (1H, br. s), 5.28-5.14 (2H, m), 7.16 (1H, t, J = 7.5 Hz), 7.39 (1H, t, J = 7.6 Hz), 7.54 (1H, d, J = 8.4 Hz), 7.89 (1H, d, J = 8.2 Hz). |
| 1-361 | 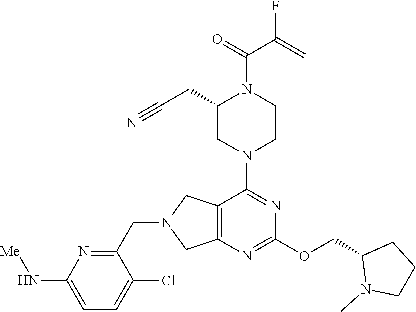 [SMILES: C=C(F)C(=O)N1CCN(c2nc(OC[C@@H]3CCCN3C)nc3c2CN(Cc2nc(NC)ccc2Cl)C3)C[C@@H]1CC#N] | S | M + 1 = 584.3 | 16%/ 61% | ¹H NMR (400 MHz, CD3OD) δ 7.40 (d, J = 8.8 Hz, 1H), 6.41 (d, J = 8.8 Hz, 1H), 5.45-5.24 (m, 2H), 4.78 (dd, J = 12.6, 3.2 Hz, 2H), 4.54-4.45 (m, 3H), 4.18 (s, 3H), 4.14 (s, 2H), 3.90-3.62 (m, 4H), 3.48- 3.33 (m, 2H), 3.29-3.17 (m, 2H), 3.04 (s, 3H), 3.02- 2.88 (m, 2H), 2.85 (s, 3H), 2.42-1.93 (m, 4H). |

Further Example Compounds

| | |
|---|---|
| 1-347 | 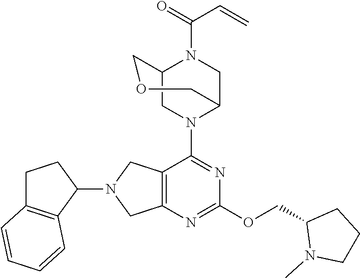 [SMILES: C=CC(=O)N1CC2COCC1CN2c1nc(OC[C@@H]2CCCN2C)nc2c1CN(C1CCc3ccccc31)C2] |
| 1-348 | 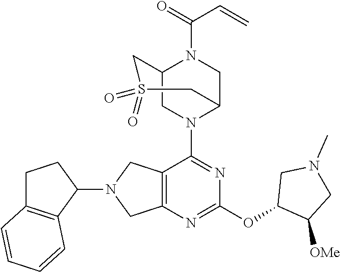 [SMILES: C=CC(=O)N1CC2CS(=O)(=O)CC1CN2c1nc(O[C@@H]2CN(C)C[C@H]2OC)nc2c1CN(C1CCc3ccccc31)C2] |
| 1-349 | 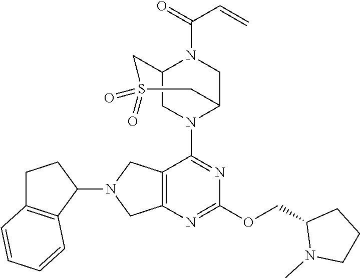 [SMILES: C=CC(=O)N1CC2CS(=O)(=O)CC1CN2c1nc(OC[C@@H]2CCCN2C)nc2c1CN(C1CCc3ccccc31)C2] |
| 1-350 | 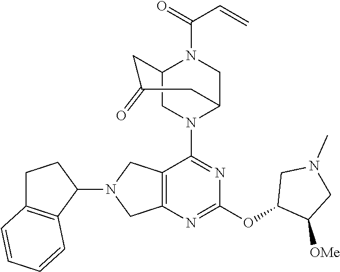 [SMILES: C=CC(=O)N1CC2CC(=O)CC1CN2c1nc(O[C@@H]2CN(C)C[C@H]2OC)nc2c1CN(C1CCc3ccccc31)C2] |
| 1-351 | 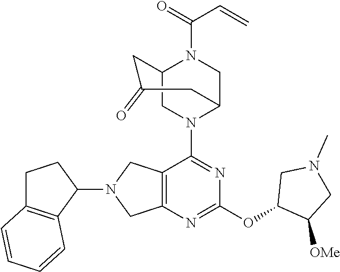 [SMILES: C=CC(=O)N1CC2CC(=O)CC1CN2c1nc(O[C@@H]2CN(C)C[C@H]2OC)nc2c1CN(C1CCc3ccccc31)C2] |
| 1-352 | 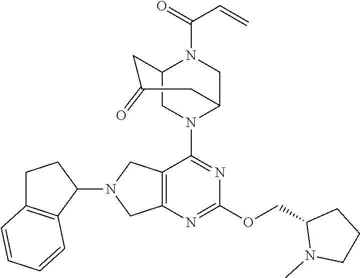 [SMILES: C=CC(=O)N1CC2CC(=O)CC1CN2c1nc(OC[C@@H]2CCCN2C)nc2c1CN(C1CCc3ccccc31)C2] |
| 1-353 | 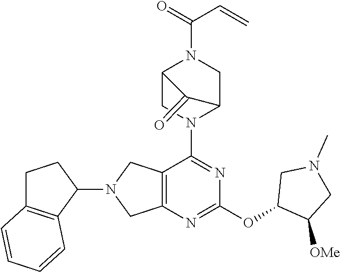 [SMILES: C=CC(=O)N1CC2C(=O)C1CN2c1nc(O[C@@H]2CN(C)C[C@H]2OC)nc2c1CN(C1CCc3ccccc31)C2] |
| 1-354 | 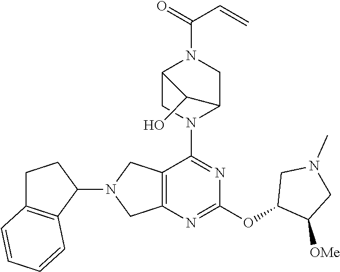 [SMILES: C=CC(=O)N1CC2C(O)C1CN2c1nc(O[C@@H]2CN(C)C[C@H]2OC)nc2c1CN(C1CCc3ccccc31)C2] |
| 1-355 | 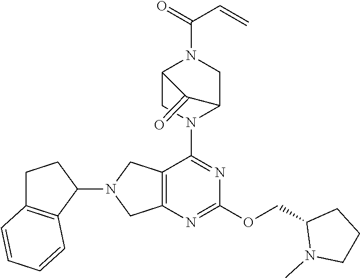 [SMILES: C=CC(=O)N1CC2C(=O)C1CN2c1nc(OC[C@@H]2CCCN2C)nc2c1CN(C1CCc3ccccc31)C2] |
| 1-356 | 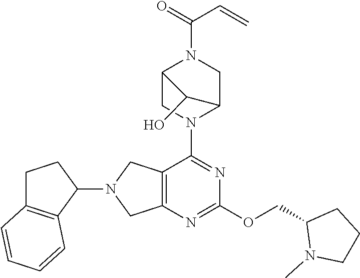 [SMILES: C=CC(=O)N1CC2C(O)C1CN2c1nc(OC[C@@H]2CCCN2C)nc2c1CN(C1CCc3ccccc31)C2] |
| 1-362 | 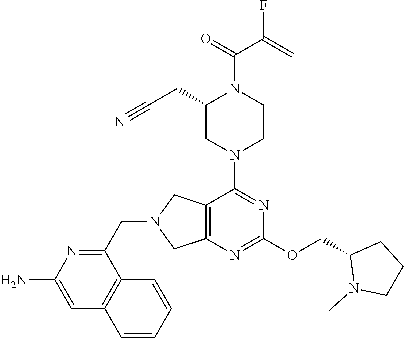 [SMILES: C=C(F)C(=O)N1CCN(c2nc(OC[C@@H]3CCCN3C)nc3c2CN(Cc2nc(N)cc4ccccc24)C3)C[C@@H]1CC#N] |
| 1-363 | 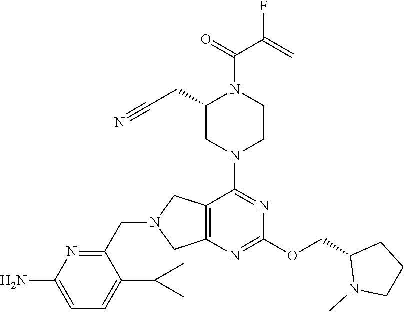 [SMILES: C=C(F)C(=O)N1CCN(c2nc(OC[C@@H]3CCCN3C)nc3c2CN(Cc2nc(N)ccc2C(C)C)C3)C[C@@H]1CC#N] |
| 1-364 | 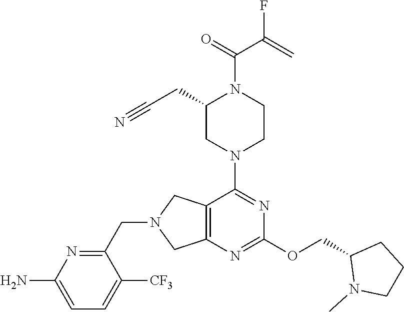 [SMILES: C=C(F)C(=O)N1CCN(c2nc(OC[C@@H]3CCCN3C)nc3c2CN(Cc2nc(N)ccc2C(F)(F)F)C3)C[C@@H]1CC#N] |
| 1-365 | 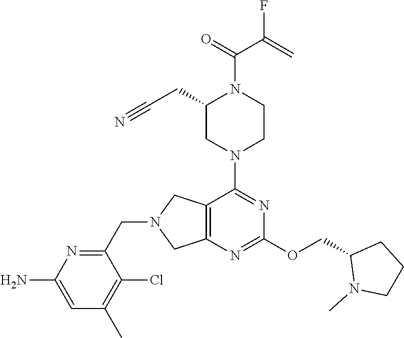 [SMILES: C=C(F)C(=O)N1CCN(c2nc(OC[C@@H]3CCCN3C)nc3c2CN(Cc2nc(N)cc(C)c2Cl)C3)C[C@@H]1CC#N] |
| 1-366 | 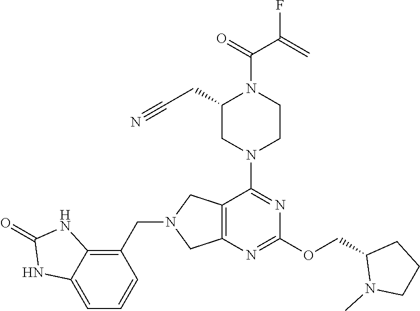 [SMILES: C=C(F)C(=O)N1CCN(c2nc(OC[C@@H]3CCCN3C)nc3c2CN(Cc2cccc4[nH]c(=O)[nH]c24)C3)C[C@@H]1CC#N] |
| 1-367 | 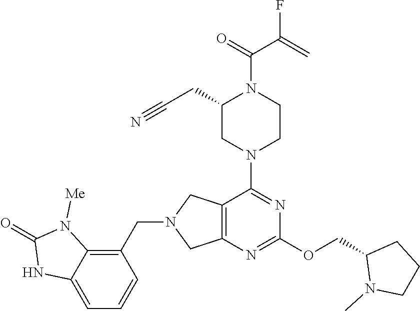 [SMILES: C=C(F)C(=O)N1CCN(c2nc(OC[C@@H]3CCCN3C)nc3c2CN(Cc2cccc4[nH]c(=O)n(C)c24)C3)C[C@@H]1CC#N] |
| 1-368 | 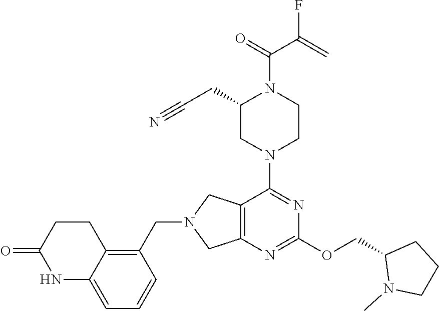 [SMILES: C=C(F)C(=O)N1CCN(c2nc(OC[C@@H]3CCCN3C)nc3c2CN(Cc2cccc4c2CCC(=O)N4)C3)C[C@@H]1CC#N] |
| 1-369 | 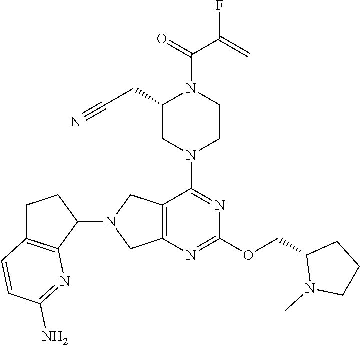 [SMILES: C=C(F)C(=O)N1CCN(c2nc(OC[C@@H]3CCCN3C)nc3c2CN(C2CCc4ccc(N)nc42)C3)C[C@@H]1CC#N] |
| 1-370 | 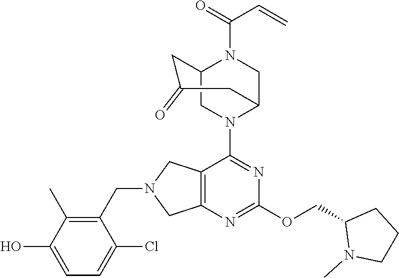 [SMILES: C=CC(=O)N1CC2CC(=O)CC1CN2c1nc(OC[C@@H]2CCCN2C)nc2c1CN(Cc1c(Cl)ccc(O)c1C)C2] |
| 1-371 | 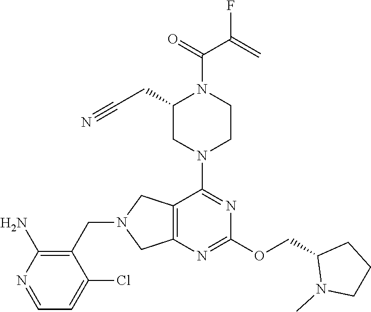 [SMILES: C=C(F)C(=O)N1CCN(c2nc(OC[C@@H]3CCCN3C)nc3c2CN(Cc2c(Cl)ccnc2N)C3)C[C@@H]1CC#N] |

### IV. General Synthetic Methods for Preparing Compounds

The following scheme can be used to practice the various embodiments disclosed herein.

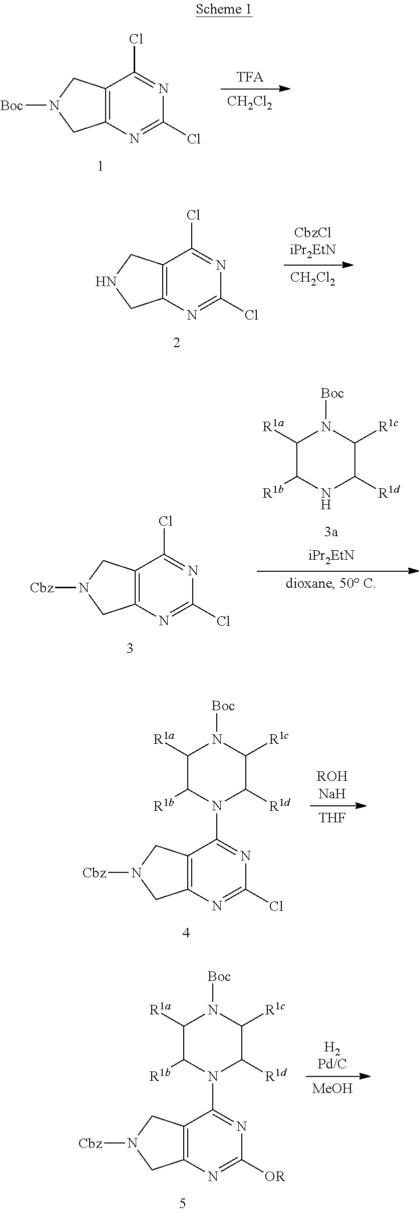

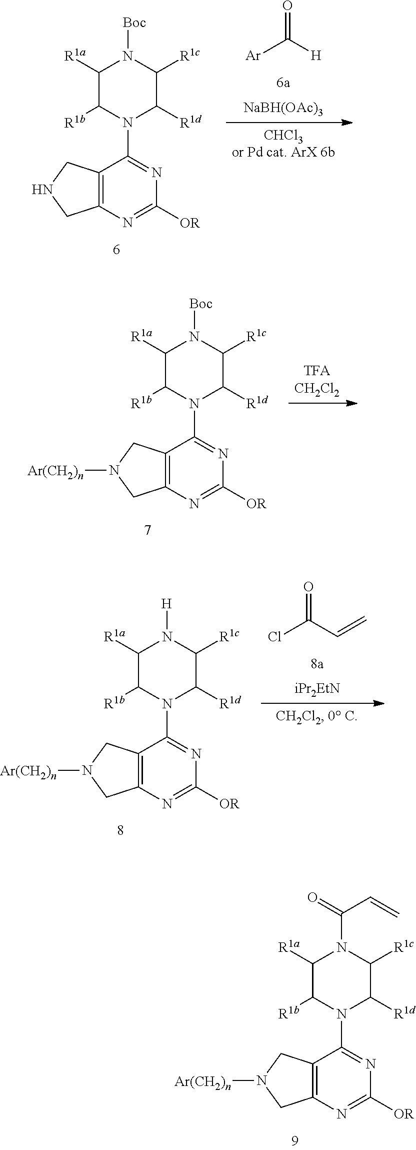

As shown in Scheme 1, tert-butyl 2,4-dichloro-5,7-dihydro-6H-pyrrolo[3,4-d]pyrimidine-6-carboxylate 1 undergoes protecting group exchange via amine 2 by Boc removal with trifluoroacetic acid, followed by Cbz protection with Cbz chloride to provide dichloro adduct 3. Regioselective addition-elimination of dichloro adduct 3 with piperazine reagent 3a provides piperazine substituted pyrimidine 4.

Continuing the synthesis, chloropyrimidine 4 is next reacted with a nucleophile such as an alcohol (ROH) in the presence of sufficient base to generate an alkoxide, thereby effecting a second addition-elimination reaction to provide nucleophile adduct 5. In embodiments, ROH is selected from:

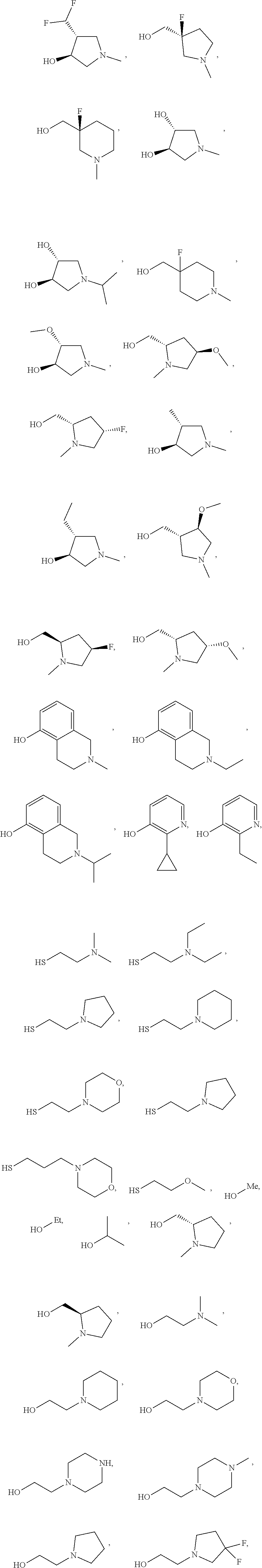

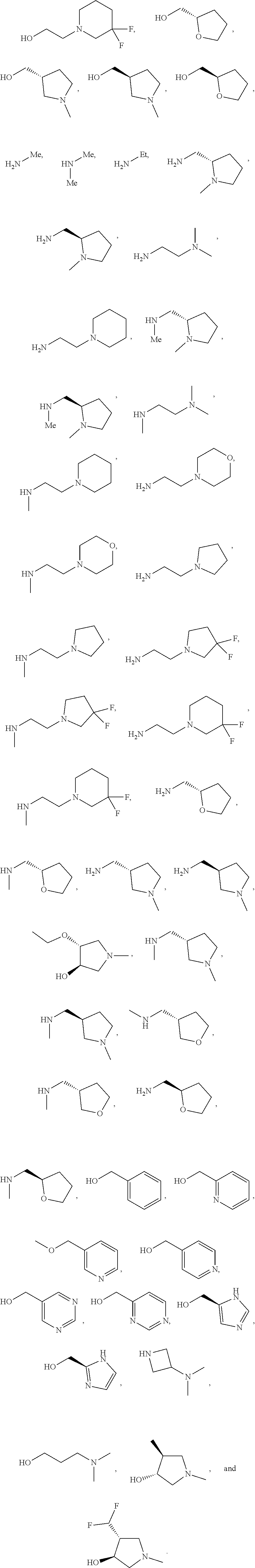

One skilled in the art will appreciate that a large array of nucleophilic partners can be used to functionalize this 2 position of the pyrimidine ring. Nucleophilic partners may include N, O, S, and C-based nucleophiles.

Next, removal of the Cbz group via hydrogenation provides amine 6. Amine 6 is an intermediate provides a jumping point for numerous analogues. In some embodiments, amine 6 can be reacted with aromatic aldehydes 6a under reductive amination conditions to provide arylated adducts 7 where n=1. In some embodiments, amine 6 can be cross-coupled with aryl halides 6b to provide arylated adducts 7, where n=0. Other arylated and even non-arylated compounds can be prepared via similar chemistries, including but not limited to, reductive amination, alkylation, vinyl halide cross-coupling, sulfonylation, amidation, to provide trialkyl amines (via either reductive amination or alkylation), vinyl amines, sulfonamides, and amides, respectively.

Installation of the reactive electrophilic acrylate group proceeds by removal of the Boc protecting group with trifluoroacetic acid to provide amine 8, followed by acrylation to afford final acrylamide adduct 9. Note that scheme I provides the parent acrylate group, but substituted acrylates may be similarly prepared.

### V. Modes of Administration

While it may be possible for the compounds disclosed herein to be administered as the raw chemical, it is also possible to present them as a pharmaceutical composition (i.e., as a formulation). Accordingly, provided herein are pharmaceutical compositions which comprise one or more of the compounds disclosed herein, or one or more pharmaceutically acceptable salts, esters, prodrugs, amides, or solvates thereof, together with one or more pharmaceutically acceptable carriers and optionally one or more other therapeutic ingredients. The carrier(s) should be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The pharmaceutical compositions may include those suitable for oral, parenteral (including subcutaneous, intradermal, intramuscular, intravenous, intraarticular, and intramedullary), administration although the most suitable route may depend upon for example the condition and disorder of the recipient. The pharmaceutical composition may conveniently be presented in unit dosage form and may be prepared by any of the methods well known in the art of pharmacy. Typically, these methods include the step of bringing into association a compound disclosed herein or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Pharmaceutical compositions of the various embodiments disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical compositions that can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores may be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds disclosed herein may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the pharmaceutical compositions described above may include other agents conventional in the art having regard to the type of formulation in question, for example those suitable for oral administration may include flavoring agents.

The compounds disclosed herein may be administered orally or via injection at a dose of from 0.1 to 500 mg/kg per day. A common dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds disclosed herein can be administered in various modes, e.g. orally, topically, or by injection. The precise amount of compound administered to a subject will be the responsibility of the attendant physician. The specific dose level for any particular subject will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for cancer involving administration of one of the compounds described herein, increased therapeutic benefit may result by also providing the patient with another therapeutic agent for cancer. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound of the various embodiments disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

### VI. Methods of Treatment

In embodiments, there are provided methods of modulating a G12C mutant K-Ras comprising contacting the G12C mutant K-Ras with a compound disclosed herein.

In embodiments, there are provided methods of treating a subject with cancer associated with a G12C Kras mutation comprising administering to the subject a compound disclosed herein in a pharmaceutically acceptable vehicle.

In embodiments, there are provided uses of a compounds disclosed herein in the manufacture of a medicament for the treatment of cancer in a subject.

Thus, in another aspect, embodiments disclosed herein provide methods for treating K-RAS-mediated disorders in a human or animal subject in need of such treatment comprising administering to said subject an amount of a compound of the various embodiments disclosed herein effective to reduce or prevent said disorder in the subject in combination with at least one additional agent for the treatment of said disorder that is known in the art. In a related aspect, the various embodiments disclosed herein provides therapeutic compositions comprising at least one compound of the various embodiments disclosed herein in combination with one or more additional agents for the treatment of K-RAS-mediated disorders. In some such embodiments, the K-RAS-mediated disease is cancer and the K-RAS presents in an oncogenic mutated form.

1. Combination Therapies

In embodiments, compounds disclosed herein may be used in combination therapies. In embodiments, the compounds disclosed herein may be used in combination with inhibitors of mammalian target of rapamycin (mTOR), insulin growth factor 1 receptor (IGF1R), and combinations thereof. Such combination therapies may be particularly suited to certain cancer types such as lung cancer. See Molinas-Areas et al. Sci. Trans. Med. 18 Sep. 2019 11:510 eaaw7999 at stm.sciencemag.org/content/1 l/510/eaaw7999. Compounds disclosed herein may be combined with modulators the ULK family of proteins, which regulate autophagy. In embodiments, the compounds disclosed herein may be used with and EGFR inhibitor. In embodiments, the compounds disclosed herein may be used with a SHP2 inhibitor.

The second agent of the pharmaceutical combination formulation or dosing regimen may have complementary activities to the compounds disclosed herein such that they do not adversely affect each other. The compounds may be administered together in a unitary pharmaceutical composition or separately. In one embodiment a compound or a pharmaceutically acceptable salt can be co-administered with a cytotoxic agent to treat proliferative diseases and cancer.

The term “co-administering” refers to either simultaneous administration, or any manner of separate sequential administration, of a compound disclosed herein or a salt thereof, and a further active pharmaceutical ingredient or ingredients, including cytotoxic agents and radiation treatment. If the administration is not simultaneous, the compounds are administered in a close time proximity to each other. Furthermore, it does not matter if the compounds are administered in the same dosage form, e.g. one compound may be administered topically and another compound may be administered orally.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound disclosed herein may be administered with another therapeutic agent simultaneously or sequentially in separate unit dosage forms or together in a single unit dosage form. Accordingly, the present invention provides a single unit dosage form comprising a compound of Formulas I-XX, an additional therapeutic agent, and a pharmaceutically acceptable carrier, adjuvant, or vehicle.

The amount of both the compound and additional therapeutic agent (in those compositions which comprise an additional therapeutic agent as described above) that may be combined with the carrier materials to produce a single dosage form will vary depending upon the host treated and the particular mode of administration. In certain embodiments, compositions of this invention are formulated such that a dosage of between 0.01-100 mg/kg body weight/day of an inventive can be administered.

Typically, any agent that has activity against a disease or condition being treated may be co-administered. Examples of such agents can be found in Cancer Principles and Practice of Oncology by V. T. Devita and S. Heilman (editors), 6^{th} edition (Feb. 15, 2001), Lippincott Williams & Wilkins Publishers. A person of ordinary skill in the art would be able to discern which combinations of agents would be useful based on the particular characteristics of the drugs and the disease involved.

In one embodiment, the treatment method includes the co-administration of a compound disclosed herein or a pharmaceutically acceptable salt thereof and at least one cytotoxic agent. The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signalling inhibitors; HDAC inhibitors, proteasome inhibitors; and inhibitors of cancer metabolism.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pflzer/Sugen), letrozole (FEMARA®, Novartis), imatinib mesylate (GLEEVEC®, Novartis), finasunate (VATALANFB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluorom ethyl ornithine (dimethylformamideO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idee), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, peefusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG₁ λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al, J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(l-methyl-piperidin-4-yl)-pyrimido[5,4-d]pyrimidine-2,8-diamine, Boehringer Ingelheim); PKI-166 ((R)-4-[4-[(l-phenylethyl)amino]-1H-pyrrolo[2,3-d]pyrimidin-6-yl]-phenol); (R)-6-(4-hydroxyphenyl)-4-[(l-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-Ml prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafamib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

In certain embodiments, chemotherapeutic agents include, but are not limited to, doxorubicin, dexamethasone, vincristine, cyclophosphamide, fluorouracil, topotecan, interferons, platinum derivatives, taxanes (e.g., paclitaxel, docetaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, trimetrexate, metoprine, cyclosporine, daunorubicin, teniposide, amphotericin, alkylating agents (e.g., chlorambucil), 5-fluorouracil, campthothecin, cisplatin, metronidazole, and imatinib mesylate, among others. In other embodiments, a compound disclosed herein is administered in combination with a biologic agent, such as bevacizumab or panitumumab.

In certain embodiments, compounds disclosed herein, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG live, bevacuzimab, fluorouracil, bexarotene, bleomycin, bortezomib, busulfan, calusterone, capecitabine, camptothecin, carboplatin, carmustine, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, elotinib, estramustine, etoposide phosphate, etoposide, exemestane, filgrastim, floxuridine, fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

Chemotherapeutic agents also include treatments for Alzheimer's Disease such as donepezil hydrochloride and rivastigmine; treatments for Parkinson's Disease such as L-DOPA/carbidopa, entacapone, ropinrole, pramipexole, bromocriptine, pergolide, trihexephendyl, and amantadine; agents for treating multiple sclerosis (MS) such as beta interferon (e.g., Avonex® and Rebif®), glatiramer acetate, and mitoxantrone; treatments for asthma such as albuterol and montelukast sodium; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

Additionally, chemotherapeutic agents include pharmaceutically acceptable salts, acids or derivatives of any of chemotherapeutic agents, described herein, as well as combinations of two or more of them.

### VII. Examples

The following Examples are provided to illustrate exemplary embodiments of the compounds disclosed herein and their preparation.

### Example 1

This Examples provides representative procedures for preparing compounds in accordance with embodiments disclosed herein.

### Representative Procedure A. Synthesis of 1-1.

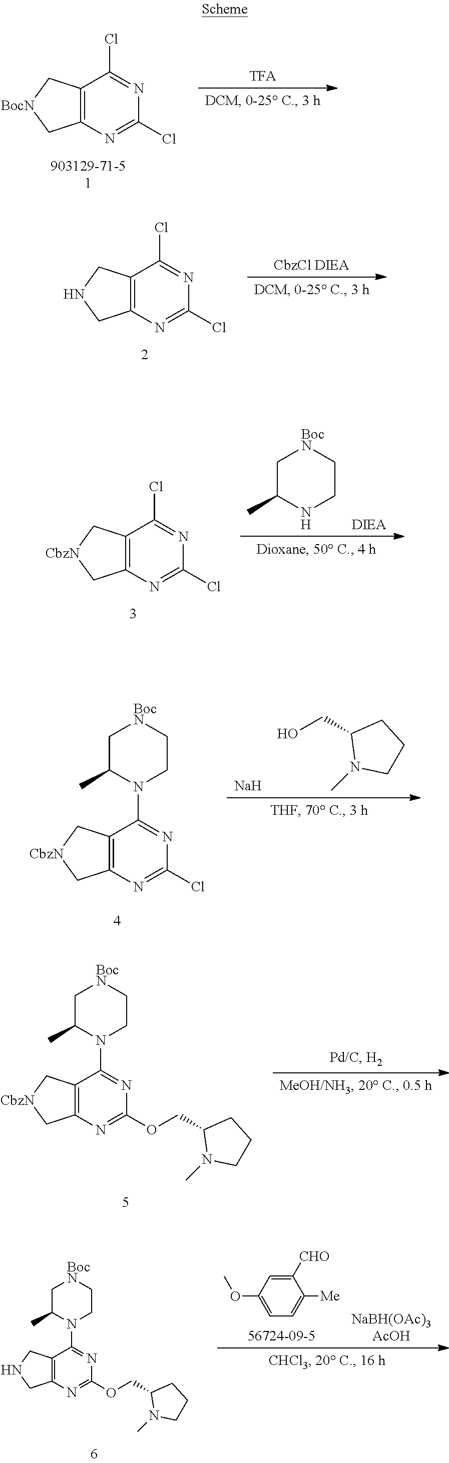

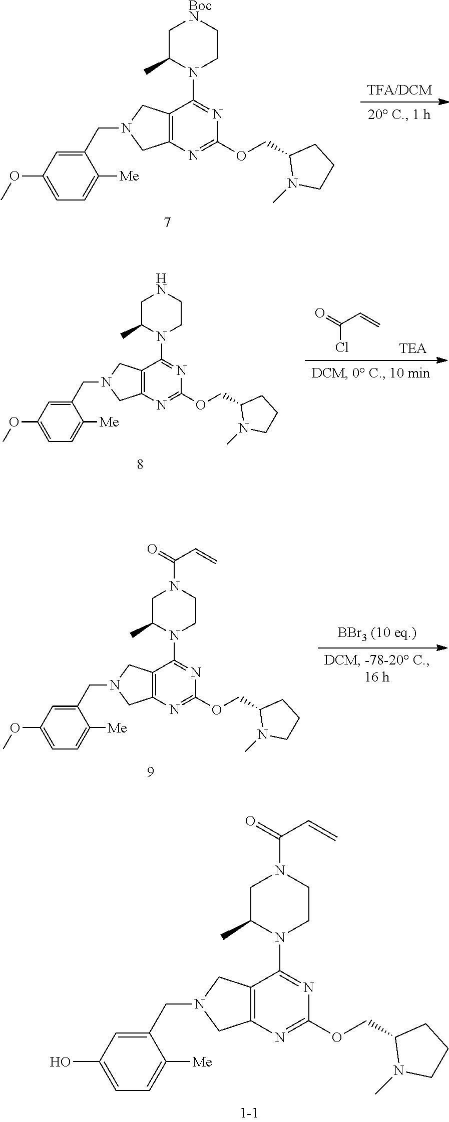

### Step 1: Synthesis of 2,4-dichloro-6,7-dihydro-5H-pyrrolo[3,4-d]pyrimidine (2)

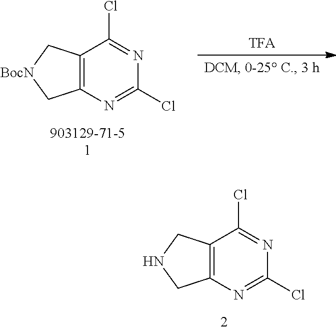 [SMILES: CC(C)(C)OC(=O)N1Cc2nc(Cl)nc(Cl)c2C1 ; "DCM, 0-25° C., 3 h" ; Clc1nc(Cl)c2c(n1)CNC2 ; "TFA"]

To a mixture of tert-butyl 2,4-dichloro-5,7-dihydro-6H-pyrrolo[3,4-d]pyrimidine-6-carboxylate (1) (5 g, 1.72 mmol, 1 equiv.) in dichloromethane (40 mL) was added trifluoroacetic acid (20 mL) slowly at 0° C. and the reaction mixture was stirred at 0-25° C. for 3 h. The reaction was concentrated under reduced pressure to give 2,4-dichloro-6,7-dihydro-5H-pyrrolo[3,4-d]pyrimidine (2) (6.0 g, crude) as white solid, which was used for the next step directly. ESI-MS m/z=190.0[M+H]⁺. Calculated MW: 188.99.

### Step 2: Synthesis of benzyl 2,4-dichloro-5,7-dihydro-6H-pyrrolo[3,4-d]pyrimidine-6-carboxylate (3)

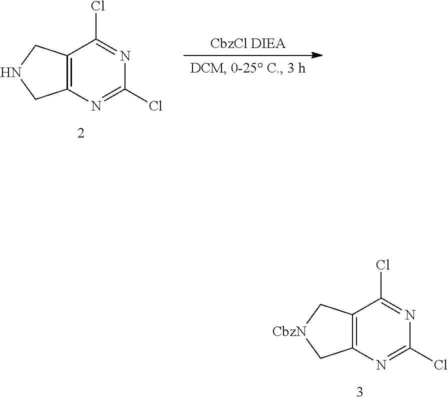 [SMILES: Clc1nc(Cl)c2c(n1)CNC2 ; "CbzCl DIEA" ; "DCM, 0-25° C., 3 h" ; O=C(OCc1ccccc1)N1Cc2nc(Cl)nc(Cl)c2C1]

To a mixture of 2,4-dichloro-6,7-dihydro-5H-pyrrolo[3,4-d]pyrimidine (2) (6.0 g, 4.2 mmol, 1 equiv.) and diisopropylethylamine (diisopropylethylamine) (27.1 g, 21 mmol, 5 equiv.) in dichloromethane (100 mL) was added benzyl chloroformate (8.6 g, 5.04 mmol, 1.2 equiv.) dropwise at 0° C. After addition, the resulting mixture was stirred under nitrogen at 25° C. for 3 h. The reaction mixture was quenched by adding water (30 mL) and extracted with dichloromethane (50 mL×3). The organic phases were combined, dried over anhydrous sodium sulfate and filtered. The filtrate was evaporated to dryness and the residue was purified by chromatography silica gel (petroleum ether/ethyl acetate=5:1) to give benzyl 2,4-dichloro-5,7-dihydro-6H-pyrrolo[3,4-d]pyrimidine-6-carboxylate (3) (5.6 g, 54.7% yield) as white solid. ESI-MS m/z=324.0 [M+H]⁺. Calculated MW: 323.02.

### Step 3: Synthesis of benzyl (S)-4-(4-(tert-butoxycarbonyl)-2-methylpiperazin-1-yl)-2-chloro-5,7-dihydro-6H-pyrrolo[3,4-d]pyrimidine-6-carboxylate (4)

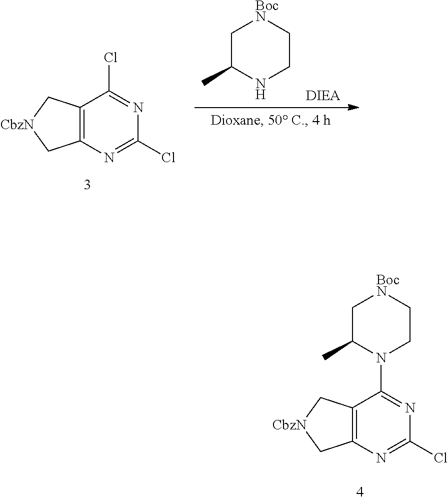

To a mixture of tert-butyl (S)-3-methylpiperazine-1-carboxylate (3) (3.70 g, 18.5 mmol, 1.2 equiv.) and diisopropylethylamine (5.97 g, 46.2 mmol, 3 equiv.) in dioxane (50 mL) was added benzyl 2,4-dichloro-5,7-dihydro-6H-pyrrolo[3,4-d]pyrimidine-6-carboxylate (5.01 g, 15.4 mmol, 1 equiv.). The flask was purged with N₂ for 3 times and the resulting mixture was stirred under N₂ at 50° C. for 4 h. To the reaction mixture was added water (30 mL) and extracted with ethyl acetate (20 mL×2). The organic phase was combined, dried over sodium sulfate, evaporated to dryness and the residue was purified by chromatography on silica gel (petroleum ether/ethyl acetate=3:1) to give benzyl (S)-4-(4-(tert-butoxycarbonyl)-2-methylpiperazin-1-yl)-2-chloro-5,7-dihydro-6H-pyrrolo[3,4-d]pyrimidine-6-carboxylate (4) (6.4 g, 85.1% yield) as white solid. ESI-MS m/z=488.2 [M+H]⁺. Calculated MW: 487.20.

### Step 4: Synthesis of benzyl 4-((S)-4-(tert-butoxycarbonyl)-2-methylpiperazin-1-yl)-2-(((S)-1-methylpyrrolidin-2-yl)methoxy)-5,7-dihydro-6H-pyrrolo[3,4-d]pyrimidine-6-carboxylate (5)

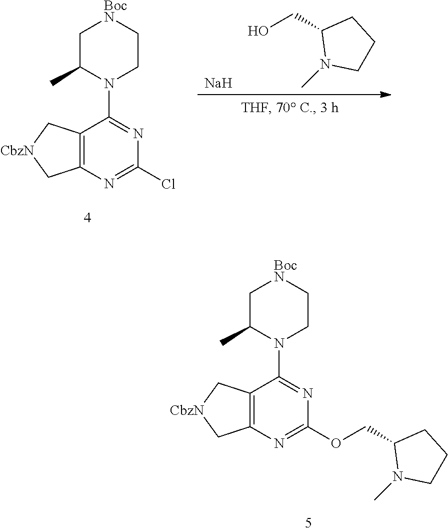

To a solution of (S)-(1-methylpyrrolidin-2-yl)methanol (4) (354 mg, 3.07 mmol, 3 equiv.) in tetrahydrofuran (tetrahydrofuran) (20 mL) was added 60% sodium hydride (82 mg, 2.05 mmol, 2 equiv.) at 0° C. After addition, the resulting mixture was stirred at 0° C. for 0.5 h. Then benzyl (S)-4-(4-(tert-butoxycarbonyl)-2-methylpiperazin-1-yl)-2-chloro-5,7-dihydro-6H-pyrrolo[3,4-d]pyrimidine-6-carboxylate (500 mg, 1.02 mmol, 1.0 equiv.) was added into the mixture under N₂ at 0° C. and the resulting mixture was stirred for 3 h at 70° C. The reaction was quenched by NH₄Cl (20 mL), extracted with ethyl acetate (ethyl acetate) (30 mL×2). The organic phase was combined, dried over anhydrous sodium sulfate, filtered and concentrated in vacuo. The residue was purified by chromatography silica gel (methanol/dichloromethane=1:10) to give benzyl 4-((S)-4-(tert-butoxycarbonyl)-2-methylpiperazin-1-yl)-2-(((S)-1-methylpyrrolidin-2-yl)methoxy)-5,7-dihydro-6H-pyrrolo[3,4-d]pyrimidine-6-carboxylate (5) (230 mg, 39.8% yield) as white solid. ESI-MS m/z=567.2 [M+H]⁺. Calculated MW: 566.32.

### Step 5: Synthesis of tert-butyl (S)-3-methyl-4-(2-(((S)-1-methylpyrrolidin-2-yl)methoxy)-6,7-dihydro-5H-pyrrolo[3,4-d]pyrimidin-4-yl)piperazine-1-carboxylate (6)

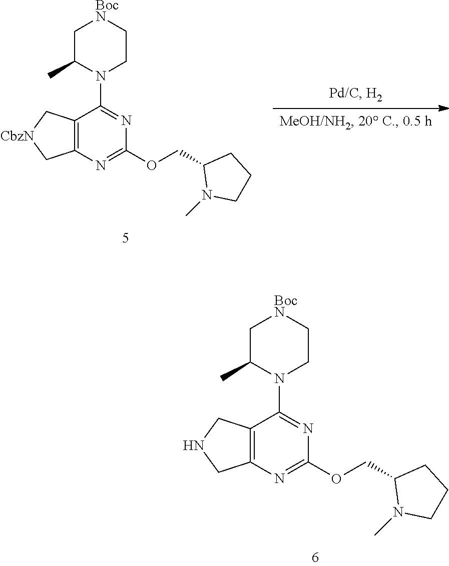 [SMILES: C[C@H]1CN(C(=O)OC(C)(C)C)CCN1c1nc(OC[C@@H]2CCCN2C)nc2c1CN(C(=O)OCc1ccccc1)C2 ; "MeOH/NH2, 20° C., 0.5 h" ; "Pd/C, H2" ; C[C@H]1CN(C(=O)OC(C)(C)C)CCN1c1nc(OC[C@@H]2CCCN2C)nc2c1CNC2]

To a solution of benzyl 4-((S)-4-(tert-butoxycarbonyl)-2-methylpiperazin-1-yl)-2-(((S)-1-methylpyrrolidin-2-yl)methoxy)-5,7-dihydro-6H-pyrrolo[3,4-d]pyrimidine-6-carboxylate (5) (230 mg, 0.406 mmol, 1 equiv.) in methanol/NH₃ (10 mL) was added 10% Pd/C (150 mg) at 20° C. After addition, the mixture was stirred under hydrogen balloon at 20° C. for 0.5 h. The mixture was filtrated and concentrated in vacuo to afford tert-butyl (S)-3-methyl-4-(2-(((S)-1-methylpyrrolidin-2-yl)methoxy)-6,7-dihydro-5H-pyrrolo[3,4-d]pyrimidin-4-yl)piperazine-1-carboxylate (6) (160 mg, crude) as colorless oil. ESI-MS m/z=433.3 [M+H]⁺. Calculated MW: 432.28.

### Step 6: Synthesis of tert-butyl (3S)-4-{6-[(5-methoxy-2-methylphenyl)methyl]-2-{[(2S)-1-methylpyrrolidin-2-yl]methoxy}-5H,7H-pyrrolo[3,4-d]pyrimidin-4-yl}-3-methylpiperazine-1-carboxylate (7)

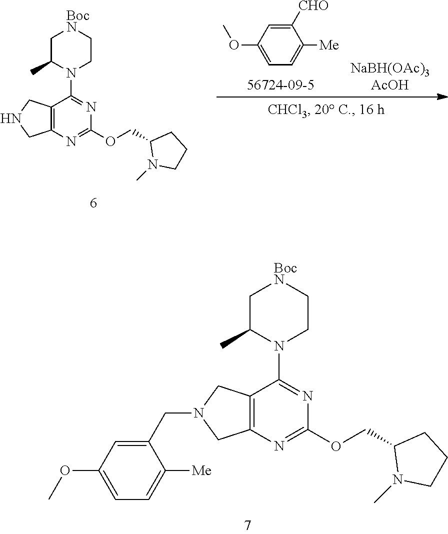

To a mixture of tert-butyl (3S)-3-methyl-4-(2-{[(2S)-1-methylpyrrolidin-2-yl]methoxy}-5H,6H,7H-pyrrolo[3,4-d]pyrimidin-4-yl)piperazine-1-carboxylate (6) (200 mg, 0.46 mmol, 1 equiv.) in dry CHCl₃ (10 mL) was added 5-methoxy-2-methylbenzaldehyde (103 mg, 0.69 mmol, 1.5 equiv.), acetic acid (42 mg, 0.69 mmol, 1.5 equiv.) and sodium triacetoxyborohydride (292 mg 1.38 mmol, 3 equiv.) at 20° C. The mixture was stirred at 20° C. for 16 h under nitrogen. The mixture was diluted with aqueous sodium bicarbonate (50 mL), extracted with 20% methanol in dichloromethane (50 mL×3). The combined organic layers were dried with anhydrous sodium sulfate, then concentrated under reduced pressure to obtain the crude product, which was purified by silica gel column chromatography (dichloromethane/methanol)=10:1 to afford tert-butyl (3S)-4-{6-[(5-methoxy-2-methylphenyl)methyl]-2-{[(2S)-1-methylpyrrolidin-2-yl]methoxy}-5H,7H-pyrrolo[3,4-d]pyrimidin-4-yl}-3-methylpiperazine-1-carboxylate (7) (242 mg, 78.9% yield) as yellow powder. ESI-MS m/z=567.4 [M+H]⁺. Calculated MW: 566.36.

### Step 7: Synthesis of (2S)-1-{6-[(5-methoxy-2-methylphenyl)methyl]-2-{[(2S)-1-methylpyrrolidin-2-yl]methoxy}-5H,7H-pyrrolo[3,4-d]pyrimidin-4-yl}-2-methylpiperazine (8)

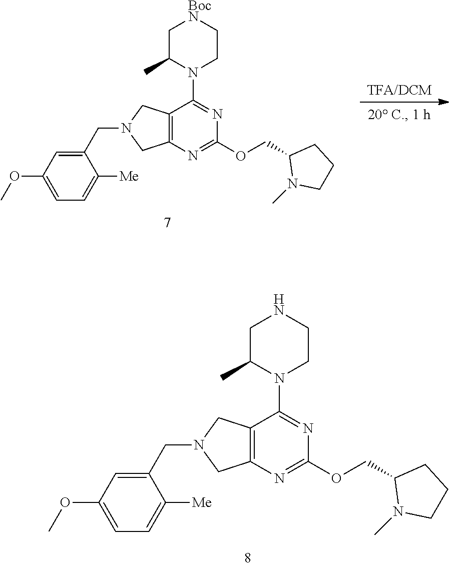 [SMILES: COc1ccc(C)c(CN2Cc3nc(OC[C@@H]4CCCN4C)nc(N4CCN(C(=O)OC(C)(C)C)C[C@@H]4C)c3C2)c1 ; "20° C., 1 h" ; "TFA/DCM" ; COc1ccc(C)c(CN2Cc3nc(OC[C@@H]4CCCN4C)nc(N4CCNC[C@@H]4C)c3C2)c1]

To a mixture of tert-butyl (3S)-4-{6-[(5-methoxy-2-methylphenyl)methyl]-2-{[(2S)-1-methylpyrrolidin-2-yl]methoxy}-5H,7H-pyrrolo[3,4-d]pyrimidin-4-yl}-3-methylpiperazine-1-carboxylate (7) (240 mg, 0.42 mmol, 1 equiv.) in dichloromethane (3 mL) was added trifluoroacetic acid (3 mL) slowly and the reaction mixture was stirred at 20° C. for 1 hour. The reaction mixture was concentrated under reduced pressure to give (2S)-1-{6-[(5-methoxy-2-methylphenyl)methyl]-2-{[(2S)-1-methylpyrrolidin-2-yl]methoxy}-5H,7H-pyrrolo[3,4-d]pyrimidin-4-yl}-2-methylpiperazine (8) (289 mg, 69.3% yield) as yellow oil, which was used for the next step directly. ESI-MS m/z=467.3 [M+H]⁺. Calculated MW: 466.31.

### Step 8: Synthesis of 1-[(3S)-4-{6-[(5-methoxy-2-methylphenyl)methyl]-2-{[(2S)-1-methylpyrrolidin-2-yl]methoxy}-5H,7H-pyrrolo[3,4-d]pyrimidin-4-yl}-3-methylpiperazin-1-yl]prop-2-en-1-one (9)

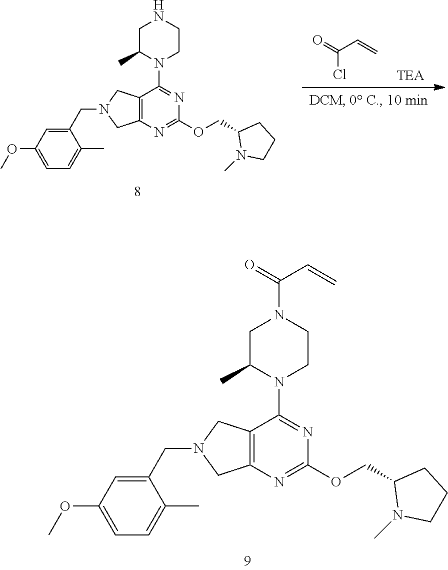

To a mixture of (2S)-1-{6-[(5-methoxy-2-methylphenyl)methyl]-2-{[(2S)-1-methylpyrrolidin-2-yl]methoxy}-5H,7H-pyrrolo[3,4-d]pyrimidin-4-yl}-2-methylpiperazine (8) (280 mg, 0.60 mmol, 1 equiv.) and triethylamine (485 mg, 4.8 mmol, 8 equiv.) in dichloromethane (8 mL) was added acryloyl chloride (81 mg, 0.89 mmol, 1.5 equiv.) dropwise at 0° C. After addition, the resulting mixture was stirred at 0° C. for 10 mins. The mixture was quenched by water (20 mL) and extracted with dichloromethane (20 mL×3). The organic phase was combined and dried over anhydrous sodium sulfate. The filtrate was concentrated after filtering. The residue was purified by Preparative-thin layer chromatography (dichloromethane/methanol)=10:1 to afford 1-[(3S)-4-{6-[(5-methoxy-2-methylphenyl)methyl]-2-{[(2S)-1-methylpyrrolidin-2-yl]methoxy}-5H,7H-pyrrolo[3,4-d]pyrimidin-4-yl}-3-methylpiperazin-1-yl]prop-2-en-1-one (9) (128 mg, 36.1% yield) as white powder. ESI-MS m/z=521.3 [M+H]⁺. Calculated MW: 520.32.

### Step 9: Synthesis of afford 1-[(3S)-4-{6-[(5-hydroxy-2-methylphenyl)methyl]-2-{[(2S)-1-methylpyrrolidin-2-yl]methoxy}-5H,7H-pyrrolo[3,4-d]pyrimidin-4-yl}-3-methylpiperazin-1-yl]prop-2-en-1-one (1-1)

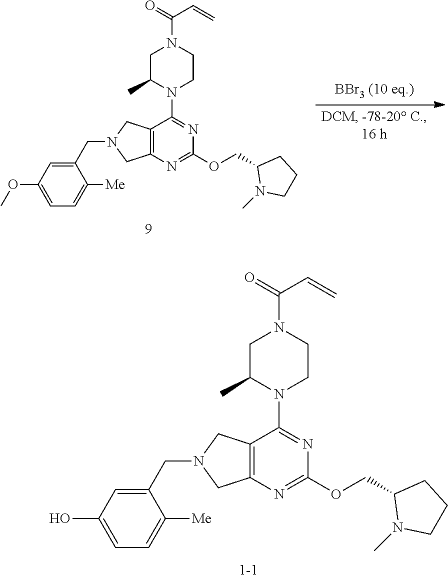 [SMILES: C=CC(=O)N1CCN(c2nc(OC[C@@H]3CCCN3C)nc3c2CN(Cc2cc(OC)ccc2C)C3)[C@@H](C)C1 ; "BBr3 (10 eq.)" ; "DCM, -78-20° C.,16 h" ; C=CC(=O)N1CCN(c2nc(OC[C@@H]3CCCN3C)nc3c2CN(Cc2cc(O)ccc2C)C3)[C@@H](C)C1]

To a mixture of 1-[(3S)-4-{6-[(5-methoxy-2-methylphenyl)methyl]-2-{[(2S)-1-methylpyrrolidin-2-yl]methoxy}-5H,7H-pyrrolo[3,4-d]pyrimidin-4-yl}-3-methylpiperazin-1-yl]prop-2-en-1-one (9) (60 mg, 0.12 mmol, 1 equiv.) in dichloromethane (10 mL) was added dropwise boron tribromide (300 mg, 1.2 mmol, 10 equiv.) at −78° C. After addition, the resulting mixture was stirred at −78° C. to 20° C. for 16 h. The mixture was quenched by water (20 mL) and extracted with dichloromethane (20 mL×3). The organic phases were combined and dried over anhydrous sodium sulfate. The organic phase was concentrated in vacuo after filtering. The residue was purified by Preparative-thin layer chromatography (dichloromethane/methanol)=8:1 to afford 1-[(3S)-4-{6-[(5-hydroxy-2-methylphenyl)methyl]-2-{[(2S)-1-methylpyrrolidin-2-yl]methoxy}-5H,7H-pyrrolo[3,4-d]pyrimidin-4-yl}-3-methylpiperazin-1-yl]prop-2-en-1-one (1-1) (15 mg, 24.4% yield) as yellow solid. ESI-MS m/z=507.3 [M+H]⁺. Calculated MW: 506.30.

### Representative Procedure B. Synthesis of 1-7.

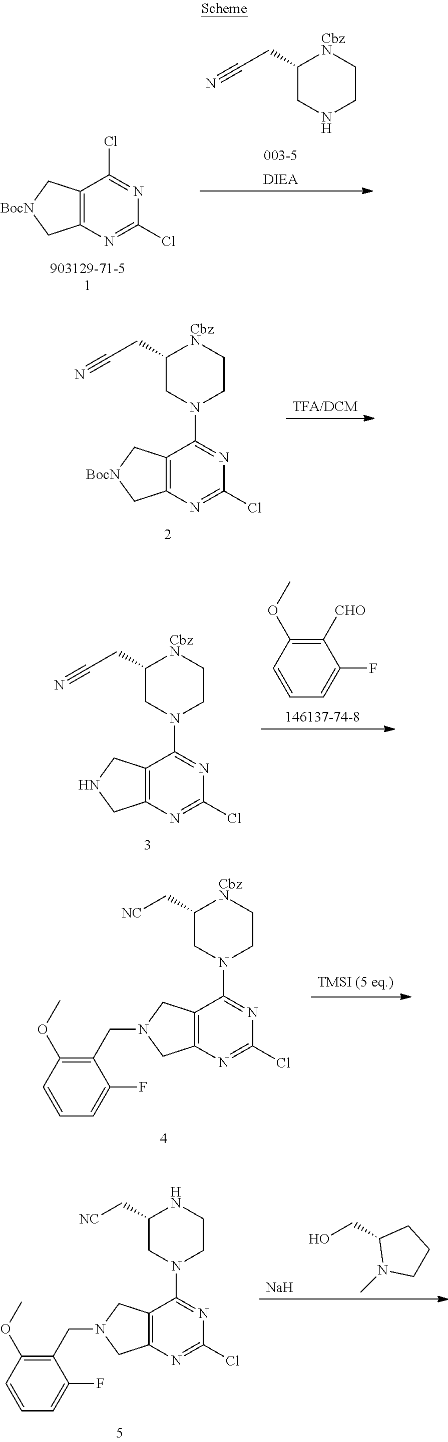

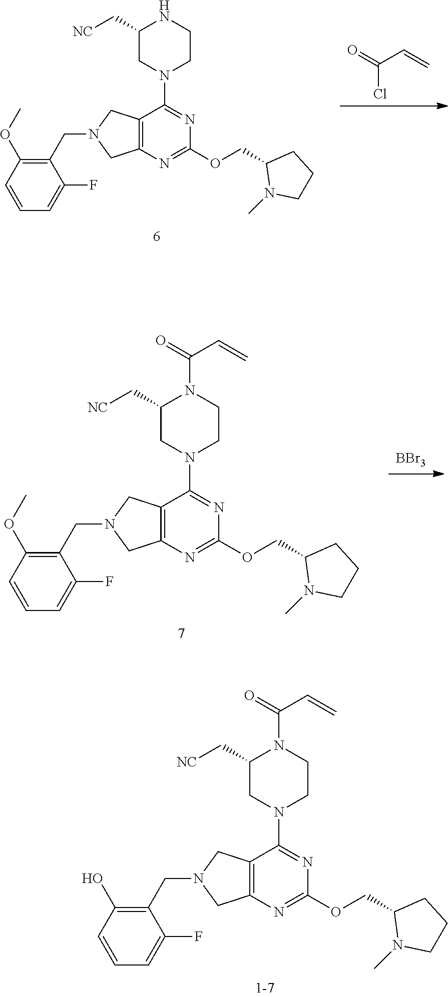

### Step 1: Synthesis of tert-butyl (S)-4-(4-((benzyloxy)carbonyl)-3-(cyanomethyl)piperazin-1-yl)-2-chloro-5,7-dihydro-6H-pyrrolo[3,4-d]pyrimidine-6-carboxylate (2)

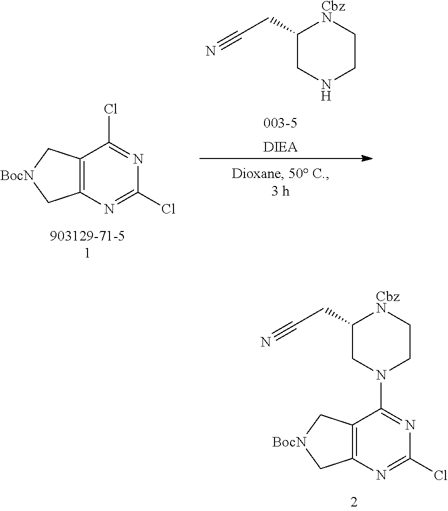 [SMILES: N#CC[C@H]1CNCCN1C(=O)OCc1ccccc1 ; CC(C)(C)OC(=O)N1Cc2nc(Cl)nc(Cl)c2C1 ; "DIEA" ; CC(C)(C)OC(=O)N1Cc2nc(Cl)nc(N3CCN(C(=O)OCc4ccccc4)[C@@H](CC#N)C3)c2C1]

To a solution of tert-butyl 2,4-dichloro-5H,7H-pyrrolo[3,4-d]pyrimidine-6-carboxylate (1) (2.74 g, 9.4 mmol, 1 equiv.) in dioxane (30 mL) was added benzyl (S)-2-(cyanomethyl)piperazine-1-carboxylate (2.4 g, 9.4 mmol, 1 equiv.) and diisopropylethylamine (5 mL). The reaction mixture was stirred at 50° C. for 3 h. The reaction was directly concentrated in vacuo. The residue was purified by chromatography silica gel (petroleum ether:ethyl acetate=3:1) to give tert-butyl (S)-4-(4-((benzyloxy)carbonyl)-3-(cyanomethyl)piperazin-1-yl)-2-chloro-5,7-dihydro-6H-pyrrolo[3,4-d]pyrimidine-6-carboxylate (2) (4.47 g, 88.9% yield) as white solid. ESI-MS m/z=513.1 [M+H]⁺. Calculated MW: 512.19.

### Step 2: Synthesis of benzyl (S)-4-(2-chloro-6,7-dihydro-5H-pyrrolo[3,4-d]pyrimidin-4-yl)-2-(cyanomethyl)piperazine-1-carboxylate (3)

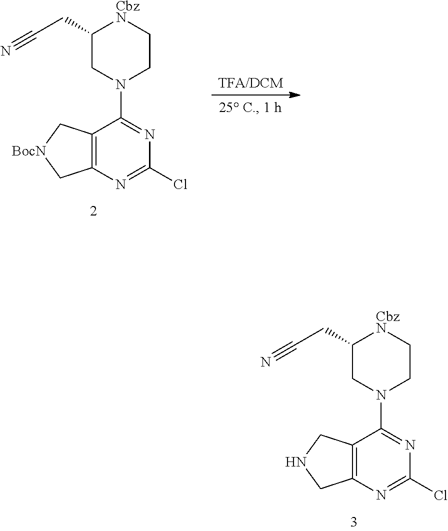 [SMILES: CC(C)(C)OC(=O)N1Cc2nc(Cl)nc(N3CCN(C(=O)OCc4ccccc4)[C@@H](CC#N)C3)c2C1 ; "25° C., 1 h" ; "TFA/DCM" ; N#CC[C@H]1CN(c2nc(Cl)nc3c2CNC3)CCN1C(=O)OCc1ccccc1]

To a solution of tert-butyl (S)-4-(4-((benzyloxy)carbonyl)-3-(cyanomethyl)piperazin-1-yl)-2-chloro-5,7-dihydro-6H-pyrrolo[3,4-d]pyrimidine-6-carboxylate (2) (1 g, 1.9 mmol) in dichloromethane (10 mL) was added trifluoroacetic acid (2 mL) at 25° C. The reaction mixture was stirred at 25° C. for 1 h. Then to the reaction mixture was added aqueous sodium bicarbonate till pH=8 and then extracted with dichloromethane (30 mL×2). The organic phases were combined, dried over sodium sulfate, filtered, and concentrated in vacuo to give benzyl (S)-4-(2-chloro-6,7-dihydro-5H-pyrrolo[3,4-d]pyrimidin-4-yl)-2-(cyanomethyl)piperazine-1-carboxylate (3) (842 mg, 72.6% yield) as white solid. ESI-MS m/z=413.2 [M+H]⁺. Calculated MW: 412.14.

### Step 3: Synthesis of benzyl (S)-4-(2-chloro-6-(2-fluoro-6-methoxybenzyl)-6,7-dihydro-5H-pyrrolo[3,4-d]pyrimidin-4-yl)-2-(cyanomethyl)piperazine-1-carboxylate (4)

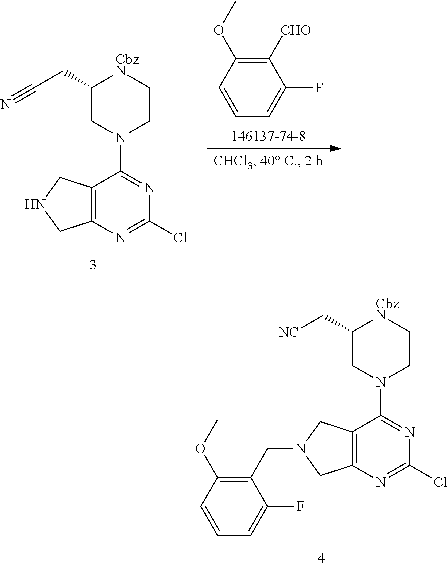

To a solution of benzyl (S)-4-(2-chloro-6,7-dihydro-5H-pyrrolo[3,4-d]pyrimidin-4-yl)-2-(cyanomethyl)piperazine-1-carboxylate (3) (742 mg, 1.80 mmol, 1 equiv.) in CHCl₃ (10 mL) was added 2-fluoro-6-methoxybenzaldehyde (277 mg, 1.80 mmol, 1 equiv.), sodium triacetoxyborohydride (1500 mg, 7.20 mmol, 4 equiv.) and acetic acid (0.5 mL). The reaction mixture was stirred at 40° C. for 2 h. The reaction quenched with water (30 mL), extracted with dichloromethane (20 mL×3). The organic phases were combined, dried over sodium sulfate, filtered, and concentrated in vacuo. The residue was purified by chromatography silica gel (dichloromethane/methanol=50:1) to give benzyl (S)-4-(2-chloro-6-(2-fluoro-6-methoxybenzyl)-6,7-dihydro-5H-pyrrolo[3,4-d]pyrimidin-4-yl)-2-(cyanomethyl)piperazine-1-carboxylate (4) (553 mg, 48.4% yield) as white solid. ESI-MS m/z=551.2 [M+H]⁺. Calculated MW: 550.19.

### Step 4: Synthesis of (S)-2-(4-(2-chloro-6-(2-fluoro-6-methoxybenzyl)-6,7-dihydro-5H-pyrrolo[3,4-d]pyrimidin-4-yl)piperazin-2-yl)acetonitrile (5)

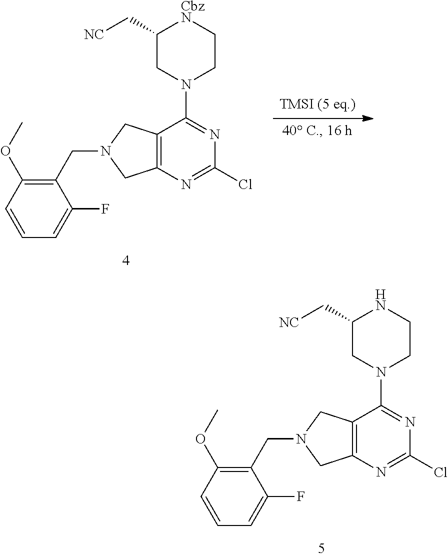 [SMILES: COc1cccc(F)c1CN1Cc2nc(Cl)nc(N3CCN(C(=O)OCc4ccccc4)[C@@H](CC#N)C3)c2C1 ; "40° C., 16 h" ; "TMSI (5 eq.)" ; COc1cccc(F)c1CN1Cc2nc(Cl)nc(N3CCN[C@@H](CC#N)C3)c2C1]

To a solution of benzyl (2S)-4-{2-chloro-6-[(2-fluoro-6-methoxyphenyl)methyl]-5H,7H-pyrrolo[3,4-d]pyrimidin-4-yl}-2-(cyanomethyl)piperazine-1-carboxylate (4) (533 mg, 0.970 mmol, 1 equiv.) in dichloromethane (10 mL) was added iodotrimethylsilane (970 mg, 4.85 mmol, 5 equiv.). The reaction mixture was stirred at 40° C. for 16 h. The reaction mixture was quenched with methanol (5 mL) and the residue was purified by preparative-thin layer chromatography (dichloromethane/methanol=10:1) to give (S)-2-(4-(2-chloro-6-(2-fluoro-6-methoxybenzyl)-6,7-dihydro-5H-pyrrolo[3,4-d]pyrimidin-4-yl)piperazin-2-yl)acetonitrile (5) (463 mg, 68.5% yield) as yellow solid. ESI-MS m/z=417.1 [M+H]⁺. Calculated MW: 416.15.

### Step 5: Synthesis of 2-((S)-4(6-(2-fluoro-6-methoxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (6)

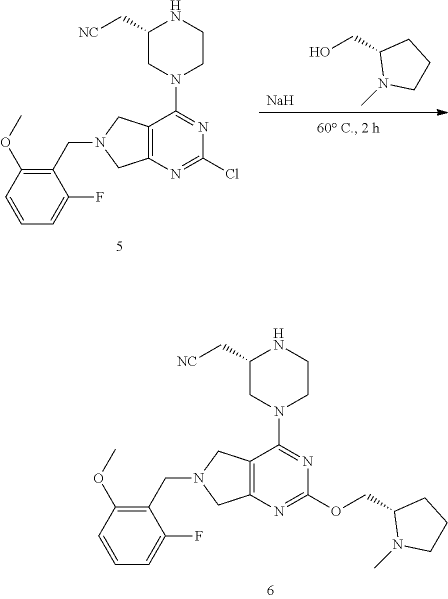

To a mixture of (S)-(1-methylpyrrolidin-2-yl) methanol (842 mg, 7.32 mmol, 4 equiv.) in tetrahydrofuran (10 mL) was added 60% sodium hydride (175 mg, 7.32 mmol, 4 equiv.) at 25° C. After addition, the resulting mixture was stirred at 25° C. for 15 min, then 2-[(2S)-4-{2-chloro-6-[(2-fluoro-6-methoxyphenyl)methyl]-5H,7H-pyrrolo[3,4-d]pyrimidin-4-yl}piperazin-2-yl]acetonitrile (5) (763 mg, 1.83 mmol, 1 equiv.) was added. After addition, the resulting mixture was stirred at 60° C. for 2 h. The reaction was quenched with water (20 mL), extracted with ethyl acetate (20 mL×3). The organic phases were combined, and dried over sodium sulfate, and filtered. The filtrate was evaporated to dryness and the residue was purified by preparative-thin layer chromatography (dichloromethane/methanol=20:1) to give 2-((S)-4-(6-(2-fluoro-6-methoxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (6) (256 mg, 25.4% yield) as yellow solid. ESI-MS m/z=496.3 [M+H]⁺. Calculated MW: 495.28.

### Step 6: Synthesis of 2-((S)-1-acryloyl-4-(6-(2-fluoro-6-methoxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (7)

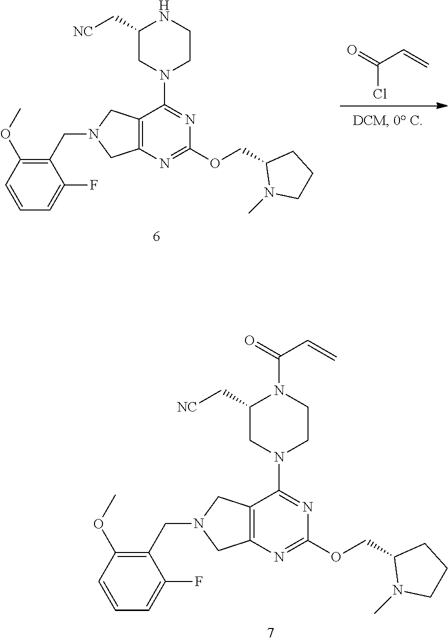

To a mixture of 2-[(2S)-4-{6-[(2-fluoro-6-methoxyphenyl)methyl]-2-{[(2S)-1-methylpyrrolidin-2-yl]methoxy}-5H,7H-pyrrolo[3,4-d]pyrimidin-4-yl}piperazin-2-yl]acetonitrile (6) (256 mg, 0.52 mmol, 1 equiv.) and triethylamine (263 mg, 2.60 mmol, 5 equiv.) in dichloromethane (5 mL) was added dropwise of acryloyl chloride (47.1 mg, 0.52 mmol, 1 equiv.) at 0° C. After addition, the resulting mixture was stirred under nitrogen at 0° C. for 0.5 h. The reaction was quenched with water (10 mL) and extracted with dichloromethane (10 mL×3). The organic phases were combined, dried over sodium sulfate and filtered. The filtrate was evaporated to dryness in vacuo and the residue was purified by preparative-thin layer chromatography (dichloromethane/methanol=10:1) to give 2-((S)-1-acryloyl-4-(6-(2-fluoro-6-methoxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (7) (160 mg, 50.4% yield) as yellow solid. ESI-MS m/z=550.3 [M+H]⁺. Calculated MW: 549.29.

### Step 7: Synthesis of 2-((S)-1-acryloyl-4-(6-(2-fluoro-6-hydroxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (1-7)

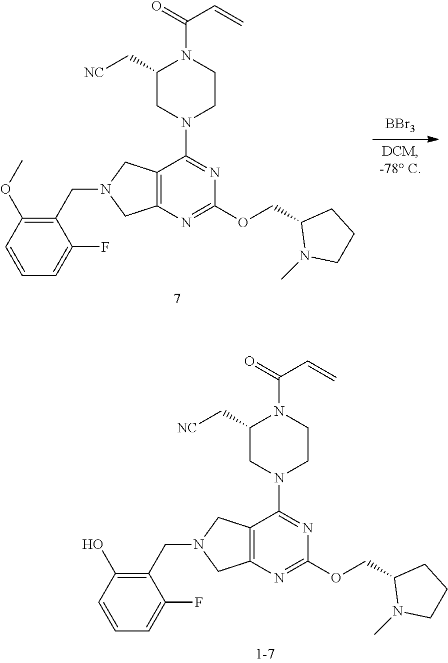 [SMILES: C=CC(=O)N1CCN(c2nc(OC[C@@H]3CCCN3C)nc3c2CN(Cc2c(F)cccc2OC)C3)C[C@@H]1CC#N ; "BBr3" ; "DCM,-78° C." ; C=CC(=O)N1CCN(c2nc(OC[C@@H]3CCCN3C)nc3c2CN(Cc2c(O)cccc2F)C3)C[C@@H]1CC#N]

To a mixture of 2-((S)-1-acryloyl-4-(6-(2-fluoro-6-methoxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (7) (140 mg, 0.250 mmol, 1 equiv.) in dichloromethane (5 mL) was added boron tribromide (626 mg, 2.50 mmol, 10 equiv.) slowly at −78° C. and the reaction mixture was stirred at −78° C. to 20° C. for 16 h. Then to the reaction mixture was added saturated sodium bicarbonate to pH=7, extracted with dichloromethane (20 mL×3). The organic phases were combined, dried over sodium sulfate, filtered and concentrated in vacuo. The residue was purified by preparative-thin layer chromatography (dichloromethane/methanol=10:1) to give 2-((S)-1-acryloyl-4-(6-(2-fluoro-6-hydroxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (1-7) (14.0 mg, 10.3% yield) as white solid. ESI-MS m/z=536.3 [M+H]⁺. Calculated MW: 535.27.

### Representative Procedure C. Synthesis of 1-9.

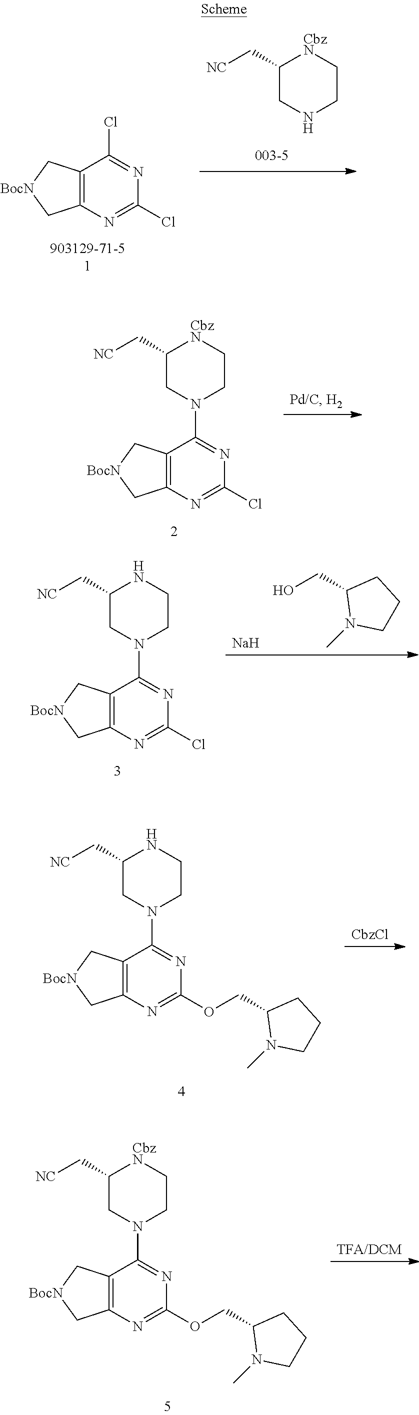

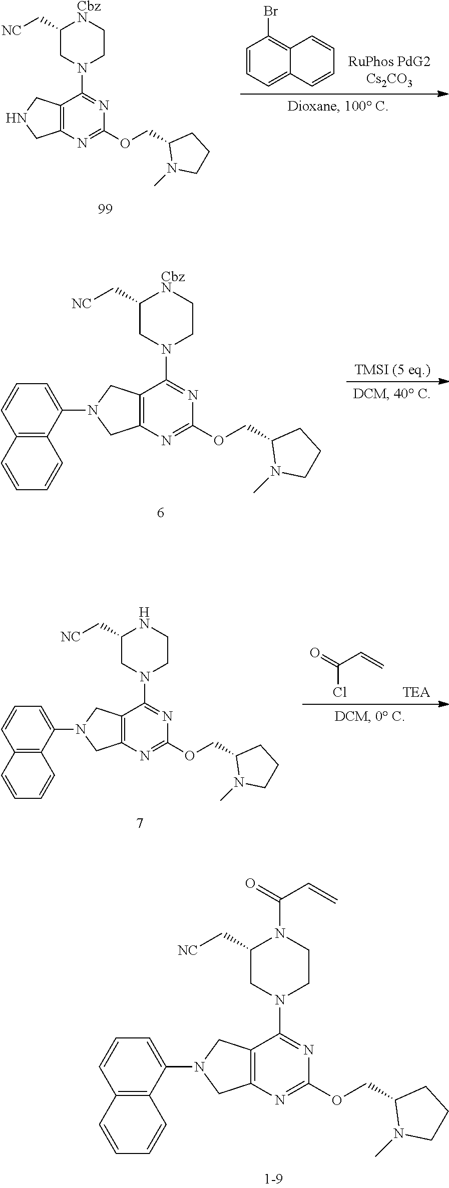

### Step 1: Synthesis of tert-butyl (S)-4-(4-((benzyloxy)carbonyl)-3-(cyanomethyl)piperazin-1-yl)-2-chloro-5,7-dihydro-6H-pyrrolo[3,4-d]pyrimidine-6-carboxylate (2)

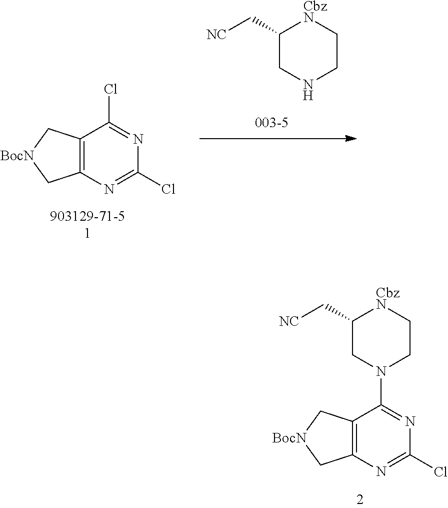 [SMILES: N#CC[C@H]1CNCCN1C(=O)OCc1ccccc1 ; CC(C)(C)OC(=O)N1Cc2nc(Cl)nc(Cl)c2C1 ; CC(C)(C)OC(=O)N1Cc2nc(Cl)nc(N3CCN(C(=O)OCc4ccccc4)[C@@H](CC#N)C3)c2C1]

To a solution of benzyl (2S)-2-(cyanomethyl)piperazine-1-carboxylate (1) (3.0 g, 11.6 mmol, 1 equiv.) and tert-butyl 2,4-dichloro-5H,7H-pyrrolo[3,4-d]pyrimidine-6-carboxylate (3.4 g, 11.6 mmol, 1 equiv.) in dioxane (30 mL) was added diisopropylethylamine (4.5 g, 34.8 mmol, 3 equiv.). The reaction mixture was stirred at 50° C. for 1 h. The reaction was concentrated in vacuo and the residue was purified by flash chromatography (petroleum ether/ethyl acetate=5:1) to give tert-butyl (S)-4-(4-((benzyloxy)carbonyl)-3-(cyanomethyl)piperazin-1-yl)-2-chloro-5,7-dihydro-6H-pyrrolo[3,4-d]pyrimidine-6-carboxylate (2) (5.1 g, 80.2% yield) as a colorless oil. ¹H NMR (400 MHz, CDCl3) δ 7.34 (d, J=11.6 Hz, 4H), 4.67-4.37 (m, 1H), 4.04 (s, 3H), 2.84 (d, J=183.3 Hz, 5H), 1.47 (s, 9H).

### Step 2: Synthesis of tert-butyl 2-chloro-4-[(3S)-3-(cyanomethyl)piperazin-1-yl]-5H,7H-pyrrolo[3,4-d]pyrimidine-6-carboxylate (3)

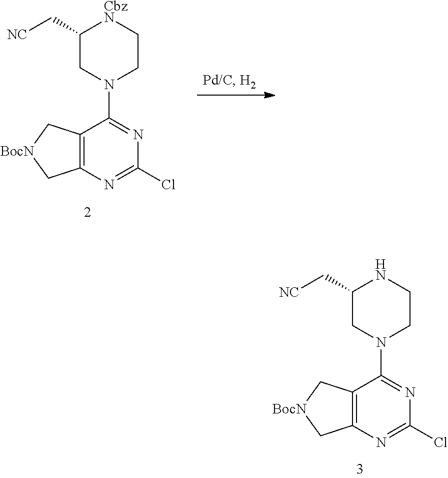 [SMILES: CC(C)(C)OC(=O)N1Cc2nc(Cl)nc(N3CCN(C(=O)OCc4ccccc4)[C@@H](CC#N)C3)c2C1 ; "Pd/C, H2" ; CC(C)(C)OC(=O)N1Cc2nc(Cl)nc(N3CCN[C@@H](CC#N)C3)c2C1]

To a solution of benzyl (2S)-4-{6-[(tert-butoxy)carbonyl]-2-chloro-5H,7H-pyrrolo[3,4-d]pyrimidin-4-yl}-2-(cyanomethyl)piperazine-1-carboxylate (2) (1.0 g, 2.0 mmol, 1 equiv.) in tetrahydrofuran (15 mL) was added 10% Pd/C (300 mg). The reaction mixture was stirred at 20° C. for 2 h. The mixture was filtered and concentrated in vacuo. The residue was purified by flash chromatography (petroleum ether/ethyl acetate=1:1) to give tert-butyl 2-chloro-4-[(3S)-3-(cyanomethyl)piperazin-1-yl]-5H,7H-pyrrolo[3,4-d]pyrimidine-6-carboxylate (3) (650 mg, 80.2% yield) as a colorless oil. ESI-MS m/z=379.1 [M+H]⁺. Calculated MW: 378.16

### Step 3: Synthesis of tert-butyl 4-[(3S)-3-(cyanomethyl)piperazin-1-yl]-2-{[(2S)-1-methylpyrrolidin-2-yl]methoxy}-5H,7H-pyrrolo[3,4-d]pyrimidine-6-carboxylate (4)

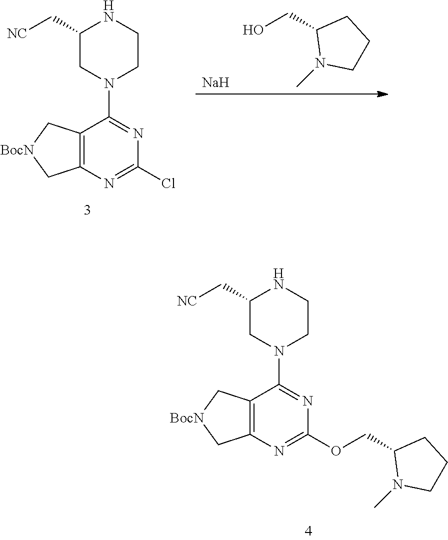

To a solution of [(2S)-1-methylpyrrolidin-2-yl]methanol (1.0 g, 6.5 mmol, 5 equiv.) in tetrahydrofuran (15 mL) was added 60% sodium hydride (240 mg, 6.5 mmol, 5 equiv.). The reaction mixture was stirred at 20° C. for 10 min. Then tert-butyl 2-chloro-4-[(3S)-3-(cyanomethyl)piperazin-1-yl]-5H,7H-pyrrolo[3,4-d]pyrimidine-6-carboxylate (3) (593 mg, 1.3 mmol, 1.05 equiv.) was added. The reaction mixture was stirred at 60° C. for 2 h. The reaction was quenched with saturated NH₄Cl (20 mL), extracted with dichloromethane (15 mL*3). The organic layers were combined, dried over anhydrous sodium sulfate, filtered and concentrated in vacuo. The residue was purified by flash chromatography (dichloromethane/methanol=10:1) to give tert-butyl 4-[(3S)-3-(cyanomethyl)piperazin-1-yl]-2-{[(2S)-1-methylpyrrolidin-2-yl]methoxy}-5H,7H-pyrrolo[3,4-d]pyrimidine-6-carboxylate (4) (701 mg, 83.7% yield) as a yellow oil. ESI-MS m/z=458.2 [M+H]⁺. Calculated MW: 457.28

### Step 4: Synthesis of benzyl (2S)-4-{6-[(tert-butoxy)carbonyl]-2-{[(2S)-1-methylpyrrolidin-2-yl]methoxy}-5H,7H-pyrrolo[3,4-d]pyrimidin-4-yl}-2-(cyanomethyl)piperazine-1-carboxylate (5)

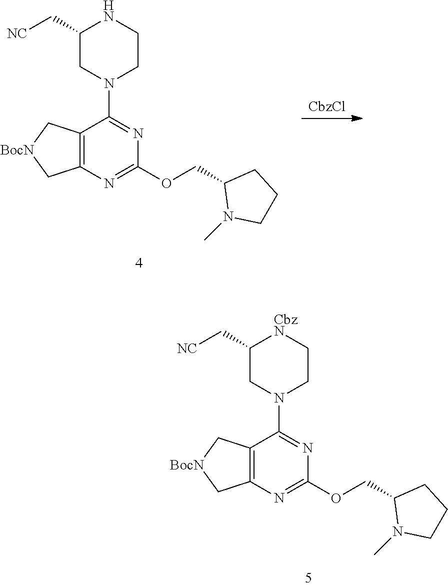

To a solution of tert-butyl 4-[(3S)-3-(cyanomethyl)piperazin-1-yl]-2-{[(2S)-1-methylpyrrolidin-2-yl]methoxy}-5H,7H-pyrrolo[3,4-d]pyrimidine-6-carboxylate (4) (600 mg, 1.3 mmol, 1 equiv.) in tetrahydrofuran (5 mL) was added triethylamine (400 mg, 3.9 mmol, 3 equiv.). The reaction mixture was stirred at 20° C. for 10 min. Then benzyl chloroformate (Cbz-C1) (335 mg, 2 mmol, 1.5 equiv.) was added. The reaction mixture was stirred at 20° C. for 1 h. The reaction mixture was concentrated in vacuo and the residue was purified by silica gel column chromatography (dichloromethane/methanol=10:1) to give benzyl (2S)-4-{6-[(tert-butoxy)carbonyl]-2-{[(2S)-1-methylpyrrolidin-2-yl]methoxy}-5H,7H-pyrrolo[3,4-d]pyrimidin-4-yl}-2-(cyanomethyl)piperazine-1-carboxylate (5) (720 mg, 87.3% yield) as a yellow oil. ESI-MS m/z=592.3 [M+H]⁺. Calculated MW: 591.32

### Step 5: Synthesis of benzyl (2S)-2-(cyanomethyl)-4-(2-{[(2S)-1-methylpyrrolidin-2-yl]methoxy}-5H,6H,7H-pyrrolo[3,4-d]pyrimidin-4-yl)piperazine-1-carboxylate (99)

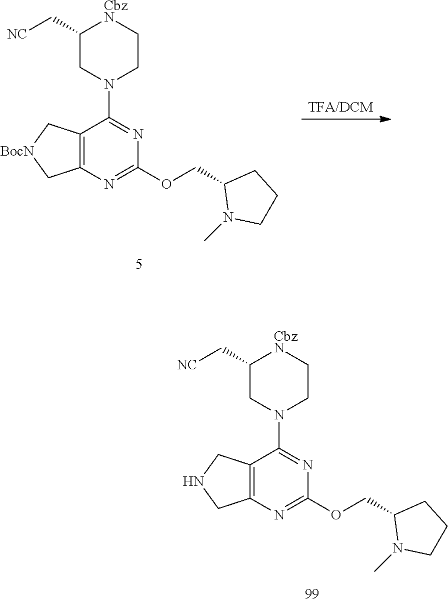 [SMILES: CN1CCC[C@H]1COc1nc2c(c(N3CCN(C(=O)OCc4ccccc4)[C@@H](CC#N)C3)n1)CN(C(=O)OC(C)(C)C)C2 ; "TFA/DCM" ; CN1CCC[C@H]1COc1nc2c(c(N3CCN(C(=O)OCc4ccccc4)[C@@H](CC#N)C3)n1)CNC2]

To a solution of benzyl (2S)-4-{6-[(tert-butoxy)carbonyl]-2-{[(2S)-1-methylpyrrolidin-2-yl]methoxy}-5H,7H-pyrrolo[3,4-d]pyrimidin-4-yl}-2-(cyanomethyl)piperazine-1-carboxylate (5) (500 mg, 0.85 mmol, 1 equiv.) in dichloromethane (4.5 mL) was added trifluoroacetic acid (1.5 mL). The reaction mixture was stirred at 20° C. for 3 h. The reaction mixture was concentrated in vacuo and to the residue was added dichloromethane (30 mL), the pH adjusted with saturated NaHCO₃ (30 mL). The organic layer was dried over anhydrous sodium sulfate, filtered and concentrated in vacuo. The residue was purified by column chromatography on silica gel (dichloromethane/methanol=5:1) to give benzyl (2S)-2-(cyanomethyl)-4-(2-{[(2S)-1-methylpyrrolidin-2-yl]methoxy}-5H,6H,7H-pyrrolo[3,4-d]pyrimidin-4-yl)piperazine-1-carboxylate (99) (401 mg, 81.5% yield) as a yellow oil. ESI-MS m/z=492.3[M+H]⁺. Calculated MW: 491.26

### Step 6: Synthesis of benzyl (2S)-2-(cyanomethyl)-4-(2-{[(2S)-1-methylpyrrolidin-2-yl]methoxy}-6-(naphthalen-1-yl)-5H,7H-pyrrolo[3,4-d]pyrimidin-4-yl)piperazine-1-carboxylate (6)

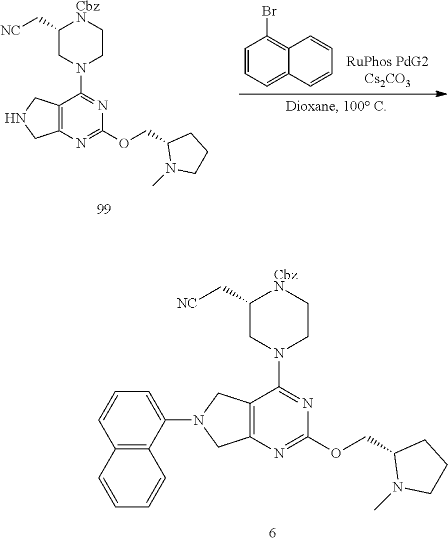

To a solution of 1-bromonaphthalene, benzyl (2S)-2-(cyanomethyl)-4-(2-{[(2S)-1-methylpyrrolidin-2-yl]methoxy}-5H,6H,7H-pyrrolo[3,4-d]pyrimidin-4-yl)piperazine-1-carboxylate (99) (500 mg, 1.02 mmol, 1 equiv.), 1-bromonaphthalene (420 mg, 2.04 mmol, 2 equiv.), and Cs₂CO₃ (280 mg, 1.5 mmol, 1.5 equiv.) in dioxane (5 mL) was added Chloro(2-dicyclohexylphosphino-2′,6′-diisopropoxy-1,1′-biphenyl)[2-(2′-amino-1,1′-biphenyl)]palladium(II) (RuPhos Pd G2) (115 mg, 0.31 mmol, 0.3 equiv.). The reaction mixture was stirred at 100° C. for 2 h. Then the mixture was concentrated in vacuo and to the residue was added water (15 mL), extracted with dichloromethane (10 mL*3). The organic layers were combined, dried over anhydrous sodium sulfate, filtered and concentrated in vacuo. The residue was purified by preparative-thin layer chromatography (dichloromethane/methanol=10:1) to give benzyl (2S)-2-(cyanomethyl)-4-(2-{[(2S)-1-methylpyrrolidin-2-yl]methoxy}-6-(naphthalen-1-yl)-5H,7H-pyrrolo[3,4-d]pyrimidin-4-yl)piperazine-1-carboxylate (6) (185 mg, 25.6% yield) as yellow oil. ESI-MS m/z=618.3[M+H]⁺. Calculated MW: 617.31.

### Step 7: Synthesis of 2-[(2S)-4-(2-{[(2S)-1-methylpyrrolidin-2-yl]methoxy}-6-(naphthalen-1-yl)-5H,7H-pyrrolo[3,4-d]pyrimidin-4-yl)piperazin-2-yl]acetonitrile (7)

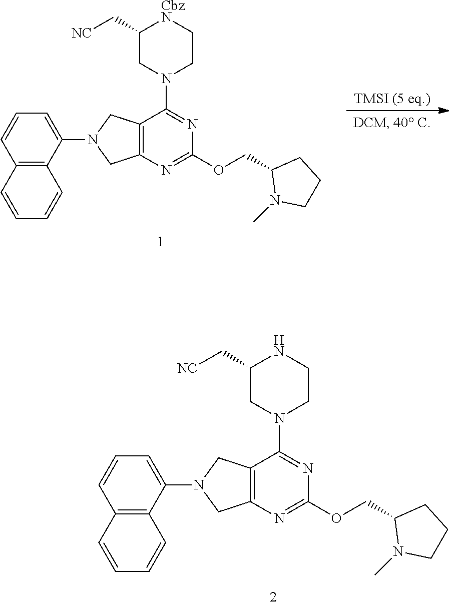 [SMILES: CN1CCC[C@H]1COc1nc2c(c(N3CCN(C(=O)OCc4ccccc4)[C@@H](CC#N)C3)n1)CN(c1cccc3ccccc13)C2 ; "DCM, 40° C." ; "TMSI (5 eq.)" ; CN1CCC[C@H]1COc1nc2c(c(N3CCN[C@@H](CC#N)C3)n1)CN(c1cccc3ccccc13)C2]

To a solution of benzyl (2S)-2-(cyanomethyl)-4-(2-{[(2S)-1-methylpyrrolidin-2-yl]methoxy}-6-(naphthalen-1-yl)-5H,7H-pyrrolo[3,4-d]pyrimidin-4-yl)piperazine-1-carboxylate (6) (185 mg, 0.30 mmol, 1 equiv.) in dichloromethane (5 mL) was added trimethylsilyl iodide (0.3 mL). The reaction mixture was stirred at 40° C. for 12 h. Then to the reaction mixture was added methanol (3 mL). The mixture was concentrated in vacuo and the residue was purified by Preparative-thin layer chromatography (dichloromethane/methanol=10:1) to give 2-[(2S)-4-(2-{[(2S)-1-methylpyrrolidin-2-yl]methoxy}-6-(naphthalen-1-yl)-5H,7H-pyrrolo[3,4-d]pyrimidin-4-yl)piperazin-2-yl]acetonitrile (7) (141 mg, 83.7% yield) as yellow oil. ESI-MS m/z=484.3[M+H]⁺. Calculated MW: 483.27.

### Step 8: Synthesis of 2-[(2S)-4-(2-{[(2S)-1-methylpyrrolidin-2-yl]methoxy}-6-(naphthalen-1-yl)-5H,7H-pyrrolo[3,4-d]pyrimidin-4-yl)-1-(prop-2-enoyl)piperazin-2-yl]acetonitrile (1-9)

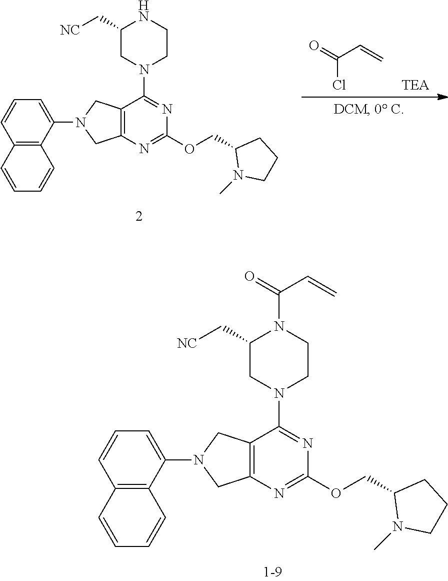

To a solution of 2-[(2S)-4-(2-{[(2S)-1-methylpyrrolidin-2-yl]methoxy}-6-(naphthalen-1-yl)-5H,7H-pyrrolo[3,4-d]pyrimidin-4-yl)piperazin-2-yl]acetonitrile (7) (100 mg, 0.21 mmol, 1 equiv.) in dichloromethane (5 mL) was added triethylamine (0.2 mL). The reaction mixture was stirred at 0° C. for 10 min. Then to the mixture was added prop-2-enoyl chloride (29 mg, 0.32 mmol, 1.5 equiv.). The reaction mixture was stirred at 0° C. for 10 min. To the reaction mixture was added water (10 mL), extracted with dichloromethane (10 mL*3). The organic layers were combined, dried over anhydrous sodium sulfate, filtered and concentrated in vacuo. The residue was purified by preparative-thin layer chromatography (dichloromethane/methanol=10:1) to give 2-[(2S)-4-(2-{[(2S)-1-methylpyrrolidin-2-yl]methoxy}-6-(naphthalen-1-yl)-5H,7H-pyrrolo[3,4-d]pyrimidin-4-yl)-1-(prop-2-enoyl)piperazin-2-yl]acetonitrile (1-9) (98 mg, 85.05% yield) as white solid. ESI-MS m/z=538.3[M+H]⁺. Calculated MW: 537.29.

### Representative Procedure D. Synthesis of 1-17.

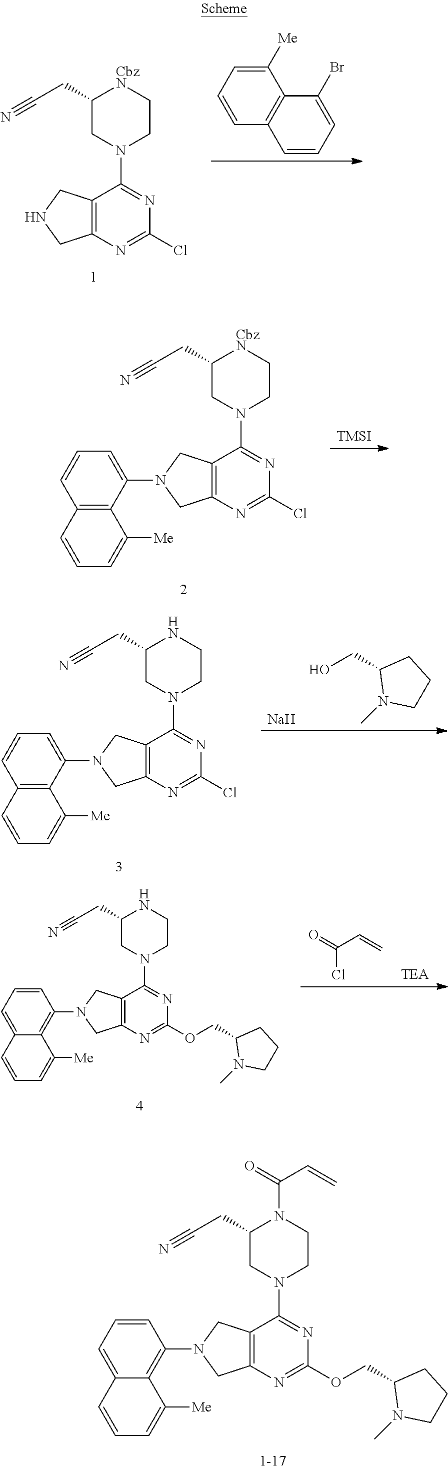

### Step 1: Synthesis of benzyl (S)-4-(2-chloro-6-(8-methylnaphthalen-1-yl)-6,7-dihydro-5H-pyrrolo[3,4-d] pyrimidin-4-yl)-2-(cyanomethyl) piperazine-1-carboxylate (2)

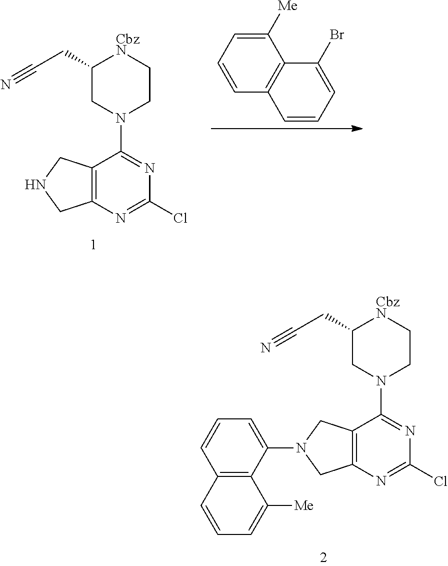 [SMILES: N#CC[C@H]1CN(c2nc(Cl)nc3c2CNC3)CCN1C(=O)OCc1ccccc1 ; Cc1cccc2cccc(Br)c12 ; Cc1cccc2cccc(N3Cc4nc(Cl)nc(N5CCN(C(=O)OCc6ccccc6)[C@@H](CC#N)C5)c4C3)c12]

To a mixture of benzyl (S)-4-(2-chloro-6,7-dihydro-5H-pyrrolo[3,4-d]pyrimidin-4-yl)-2-(cyanomethyl)piperazine-1-carboxylate (1) (500 mg, 1.2 mmol, 1 equiv., for the procedure of 1, please refer to the procedure of compound 1-7) in 1,4-dioxane (10 mL) was added 1-bromo-8-methylnaphthalene (395 mg, 1.8 mmol, 1.5 equiv.), Pd₂(dba)₃ (220 mg, 0.24 mmol, 0.2 equiv.), Xantphos (139 mg, 0.24 mmol, 0.2 equiv.), and Cs₂CO₃ (975 mg, 3 mmol, 2.5 equiv.). The mixture was stirred at 100° C. for 12 h under nitrogen. The reaction was cooled to 25° C. quenched by water (15 mL), and extracted with dichloromethane (15 mL×2). The organic phase was combined, dried over sodium sulfate, filtered and concentrated in vacuo and the residue was purified by chromatography silica gel (dichloromethane/methanol=10:1) to give benzyl (S)-4-(2-chloro-6-(8-methylnaphthalen-1-yl)-6,7-dihydro-5H-pyrrolo[3,4-d]pyrimidin-4-yl)-2-(cyanomethyl)piperazine-1-carboxylate (2) (430 mg, 64.8% yield) as yellow solid. ESI-MS m/z=553.1 [M+H]⁺. Calculated MW: 552.20.

### Step 2: Synthesis of (S)-2-(4-(2-chloro-6-(8-methylnaphthalen-1-yl)-6,7-dihydro-5H-pyrrolo[3,4-d] pyrimidin-4-yl) piperazin-2-yl) acetonitrile (3)

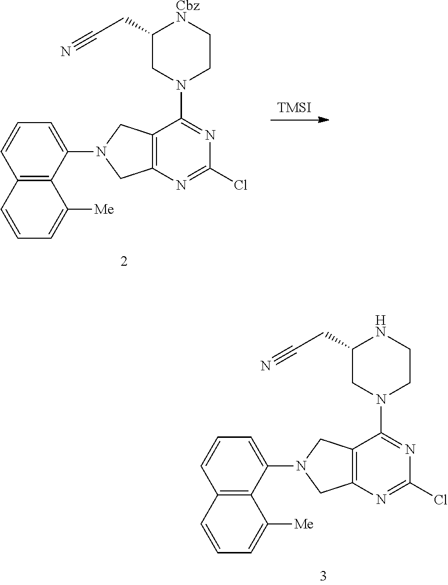 [SMILES: "TMSI" ; Cc1cccc2cccc(N3Cc4nc(Cl)nc(N5CCN(C(=O)OCc6ccccc6)[C@@H](CC#N)C5)c4C3)c12 ; Cc1cccc2cccc(N3Cc4nc(Cl)nc(N5CCN[C@@H](CC#N)C5)c4C3)c12]

To a mixture of benzyl (S)-4-(2-chloro-6-(8-methylnaphthalen-1-yl)-6,7-dihydro-5H-pyrrolo[3,4-d]pyrimidin-4-yl)-2-(cyanomethyl)piperazine-1-carboxylate (2) (430 mg, 0.78 mmol, 1 equiv.) in dichloromethane (10 mL) was added trimethylsilyl iodide (779 mg, 3.89 mmol, 5 equiv.), and the reaction mixture was stirred at 40° C. for 12 h. The mixture was quenched by methanol (2 mL), and concentrated under reduced pressure to give a residue which was purified by chromatography silica gel (dichloromethane/methanol=80:1) to give (S)-2-(4-(2-chloro-6-(8-methylnaphthalen-1-yl)-6,7-dihydro-5H-pyrrolo[3,4-d]pyrimidin-4-yl)piperazin-2-yl)acetonitrile (3) (260 mg, 90% yield) as white solid. ESI-MS m/z=419.2 [M+H]⁺. Calculated MW: 418.17.

### Step 3: Synthesis of 2-((S)-4-(6-(8-methylnaphthalen-1-yl)-2-(((S)-1-methylpyrrolidin-2-yl)methoxy)-6,7-dihydro-5H-pyrrolo[3,4-d]pyrimidin-4-yl)piperazin-2-yl)acetonitrile (4)

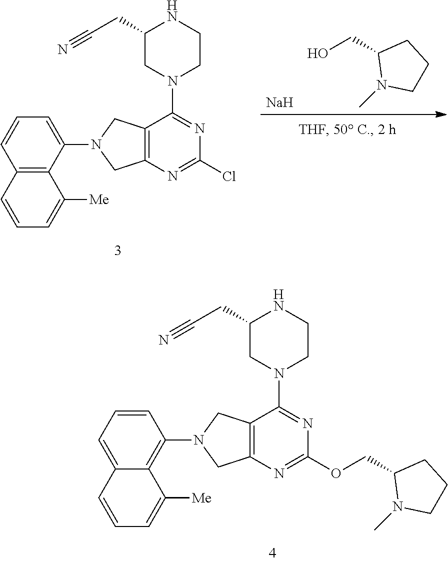

To a mixture of (S)-(1-methylpyrrolidin-2-yl)methanol (357 mg, 3.11 mmol, 5 equiv.) in tetrahydrofuran (10 mL) was added 60% sodium hydride (125 mg, 3.11 mmol, 5 equiv.) and the reaction mixture was stirred at 25° C. for 10 min, then (S)-2-(4-(2-chloro-6-(8-methylnaphthalen-1-yl)-6,7-dihydro-5H-pyrrolo[3,4-d]pyrimidin-4-yl)piperazin-2-yl)acetonitrile (3) (260 mg, 0.62 mmol, 1 equiv.) was added, and the reaction was stirred at 50° C. for 2 h. The reaction was quenched by saturated NH₄Cl (20 mL), extracted with dichloromethane (15 mL×3). The organic phase was combined, dried over sodium sulfate, filtered and concentrated in vacuo and the residue was purified by chromatography silica gel (dichloromethane/methanol=10:1) to give 2-((S)-4-(6-(8-methylnaphthalen-1-yl)-2-(((S)-1-methylpyrrolidin-2-yl)methoxy)-6,7-dihydro-5H-pyrrolo[3,4-d]pyrimidin-4-yl)piperazin-2-yl)acetonitrile (4) (170 mg, 55.2% yield) as yellow solid. ESI-MS m/z=498.3 [M+H]⁺. Calculated MW: 497.29.

### Step 4: Synthesis of 2-((S)-1-acryloyl-4-(6-(8-methylnaphthalen-1-yl)-2-(((S)-1-methylpyrrolidin-2-yl)methoxy)-6,7-dihydro-5H-pyrrolo[3,4-d]pyrimidin-4-yl)piperazin-2-yl)acetonitrile (1-17)

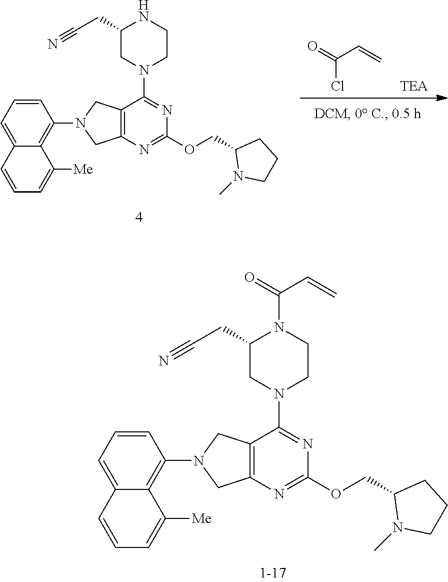

To a mixture of 2-((S)-4-(6-(8-methylnaphthalen-1-yl)-2-(((S)-1-methylpyrrolidin-2-yl)methoxy)-6,7-dihydro-5H-pyrrolo[3,4-d]pyrimidin-4-yl)piperazin-2-yl)acetonitrile (4) (170 mg, 0.34 mmol, 1 equiv.) and triethylamine (173 mg, 1.71 mmol, 5 equiv.) in dichloromethane (10 mL) was added dropwise of acryloyl chloride (48 mg, 0.51 mmol, 1.5 equiv.) at 0° C. After addition, the resulting mixture was stirred under nitrogen at 0° C. for 0.5 h. The reaction was quenched by water (10 mL), extracted with dichloromethane (10 mL×3). The organic phase was combined, dried over sodium sulfate, filtered. The filtrate was evaporated to dryness and the residue was purified by chromatography silica gel (dichloromethane/methanol=10:1) to give 2-((S)-1-acryloyl-4-(6-(8-methylnaphthalen-1-yl)-2-(((S)-1-methylpyrrolidin-2-yl)methoxy)-6,7-dihydro-5H-pyrrolo[3,4-d]pyrimidin-4-yl)piperazin-2-yl)acetonitrile (1-17) (65.8 mg, 35.1% yield) as white solid. ESI-MS m/z=552.3 [M+H]⁺. Calculated MW: 551.30.

### Representative Procedure E. Synthesis of 1-23.

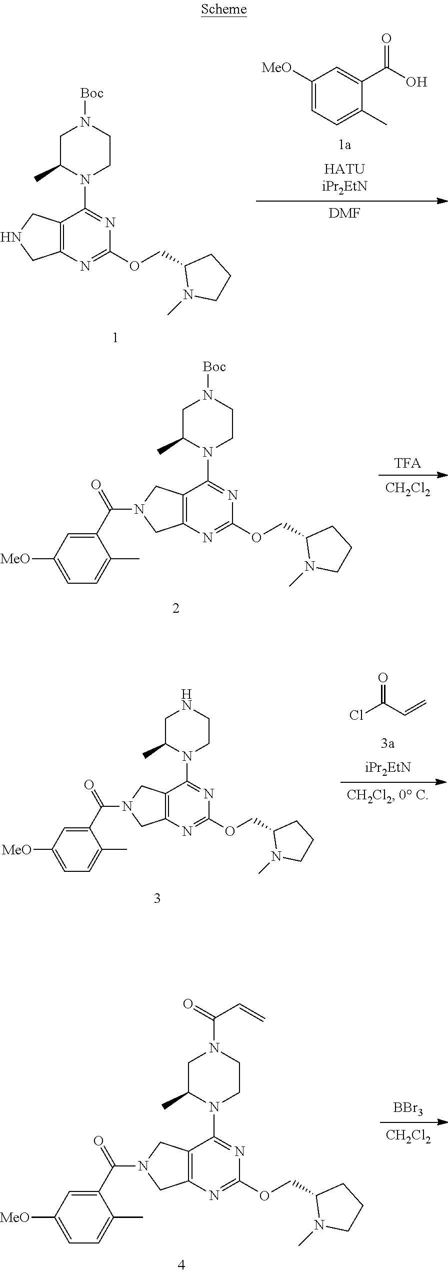

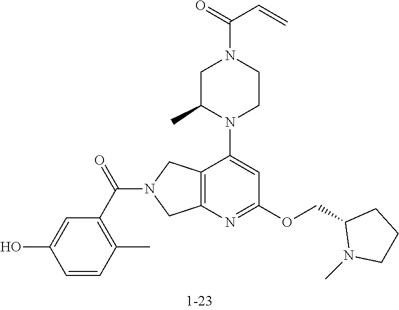 [SMILES: C=CC(=O)N1CCN(c2cc(OC[C@@H]3CCCN3C)nc3c2CN(C(=O)c2cc(O)ccc2C)C3)[C@@H](C)C1]

### Step 1: Synthesis of tert-butyl (S)-4-(6-(5-methoxy-2-methylbenzoyl)-2-(((S)-1-methylpyrrolidin-2-yl)methoxy)-6,7-dihydro-5H-pyrrolo[3,4-d]pyrimidin-4-yl)-3-methylpiperazine-1-carboxylate (2)

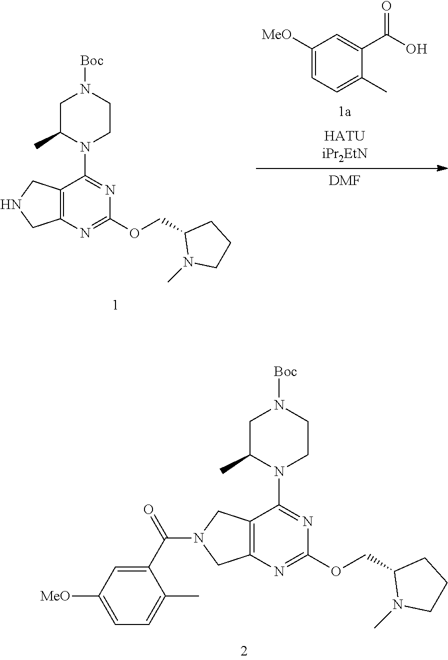

To a N,N-dimethylformamide (1.2 mL) solution of 5-methoxy-2-methylbenzoic acid (1, 30 mg, 0.18 mmol) was added 1-[bis(dimethylamino)methylene]-1H-1,2,3-triazolo[4,5-b]pyridinium 3-oxide hexafluorophosphate (91 mg, 0.24 mmol), N,N-diisopropylethylamine (0.08 mL, 0.48 mmol), and tert-butyl (S)-3-methyl-4-(2-(((S)-1-methylpyrrolidin-2-yl)methoxy)-6,7-dihydro-5H-pyrrolo[3,4-d]pyrimidin-4-yl)piperazine-1-carboxylate (1, 52 mg, 0.12 mmol) at ambient temperature. The mixture was stirred for 20 h at ambient temperature. The mixture was diluted with ethyl acetate and washed with brine three times, dried over anhydrous magnesium sulfate, and concentrated in vacuo after filtration. Purification by silica gel column chromatography (0-10% methanol in dichloromethane) gave tert-butyl (S)-4-(6-(5-methoxy-2-methylbenzoyl)-2-(((S)-1-methylpyrrolidin-2-yl)methoxy)-6,7-dihydro-5H-pyrrolo[3,4-d]pyrimidin-4-yl)-3-methylpiperazine-1-carboxylate (2) as brown oil. Yield: 68 mg (99%). MS: 581.3 [M+H]⁺.

### Step 2: Synthesis of (5-methoxy-2-methylphenyl)(4-((S)-2-methylpiperazin-1-yl)-2-(((S)-1-methylpyrrolidin-2-yl)methoxy)-5,7-dihydro-6H-pyrrolo[3,4-d]pyrimidin-6-yl)methanone (3)

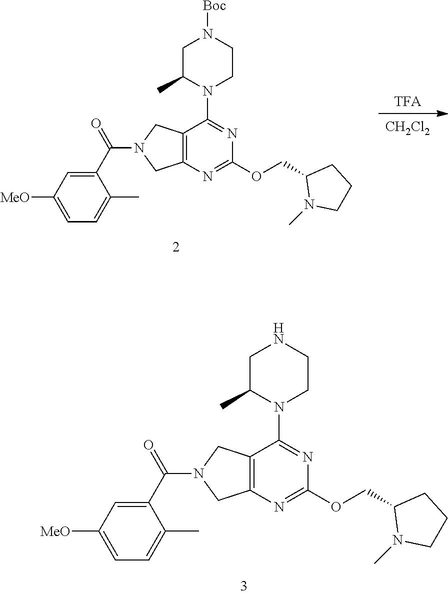 [SMILES: COc1ccc(C)c(C(=O)N2Cc3nc(OC[C@@H]4CCCN4C)nc(N4CCN(C(=O)OC(C)(C)C)C[C@@H]4C)c3C2)c1 ; "CH2Cl2" ; COc1ccc(C)c(C(=O)N2Cc3nc(OC[C@@H]4CCCN4C)nc(N4CCNC[C@@H]4C)c3C2)c1 ; "TFA"]

To a dichloromethane (1 mL) solution of tert-butyl (S)-4-(6-(5-methoxy-2-methylbenzoyl)-2-(((S)-1-methylpyrrolidin-2-yl)methoxy)-6,7-dihydro-5H-pyrrolo[3,4-d]pyrimidin-4-yl)-3-methylpiperazine-1-carboxylat (2, 68 mg, 0.12 mmol) was added trifluoroacetic acid (1 mL) at 0° C. After stirring the mixture for 1 h at room temperature, the mixture was concentrated in vacuo. The residue was loaded onto a SCX ion exchange column. The column was eluted with methanol, followed by 2N ammonia in methanol. The basic fraction was concentrated in vacuo to give (5-methoxy-2-methylphenyl)(4-((S)-2-methylpiperazin-1-yl)-2-(((S)-1-methylpyrrolidin-2-yl)methoxy)-5,7-dihydro-6H-pyrrolo[3,4-d]pyrimidin-6-yl)methanone (3) as brown oil. Yield: 45 mg (80%). MS: 481.3 [M+H]⁺.

### Step 3: Synthesis of 1-((S)-4-(6-(5-methoxy-2-methylbenzoyl)-2-(((S)-1-methylpyrrolidin-2-yl)methoxy)-6,7-dihydro-5H-pyrrolo[3,4-d]pyrimidin-4-yl)-3-methylpiperazin-1-yl)prop-2-en-1-one (4)

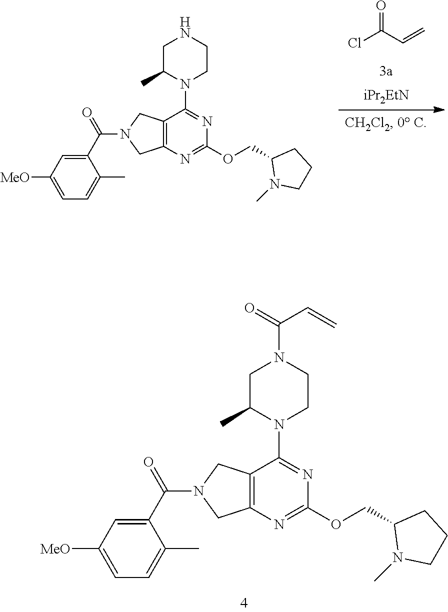

To a dichloromethane (1 mL) solution of (5-methoxy-2-methylphenyl)(4-((S)-2-methylpiperazin-1-yl)-2-(((S)-1-methylpyrrolidin-2-yl)methoxy)-5,7-dihydro-6H-pyrrolo[3,4-d]pyrimidin-6-yl)methanone (3, 45 mg, 0.090 mmol) was added N,N-diisopropylethylamine (0.14 mL, 0.14 mmol, 1M in dichloromethane) and acryloyl chloride (3a, 0.10 mL, 0.10 mmol, 1M in dichloromethane) at 0° C. After stirring the mixture for 15 minutes at 0° C., the mixture was concentrated in vacuo. Purification by preparative HPLC (C₁₈ column, 5-95% acetonitrile in water+0.1% formic acid) gave 1-((S)-4-(6-(5-methoxy-2-methylbenzoyl)-2-(((S)-1-methylpyrrolidin-2-yl)methoxy)-6,7-dihydro-5H-pyrrolo[3,4-d]pyrimidin-4-yl)-3-methylpiperazin-1-yl)prop-2-en-1-one (4) as beige solid as formic acid salt. Yield=21 mg (42%). MS 535.3 [M+H]⁺.

### Step 4: Synthesis of 1-((S)-4-(6-(5-hydroxy-2-methylbenzoyl)-2-(((S)-1-methylpyrrolidin-2-yl)methoxy)-6,7-dihydro-5H-pyrrolo[3,4-d]pyrimidin-4-yl)-3-methylpiperazin-1-yl)prop-2-en-1-one (1-23)

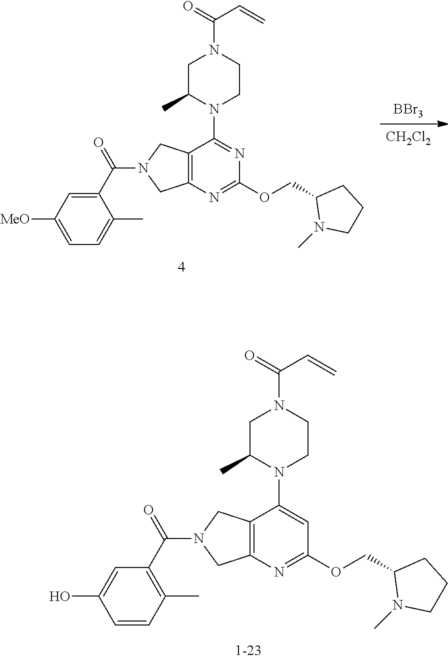 [SMILES: C=CC(=O)N1CCN(c2nc(OC[C@@H]3CCCN3C)nc3c2CN(C(=O)c2cc(OC)ccc2C)C3)[C@@H](C)C1 ; "BBr3" ; "CH2Cl2" ; C=CC(=O)N1CCN(c2cc(OC[C@@H]3CCCN3C)nc3c2CN(C(=O)c2cc(O)ccc2C)C3)[C@@H](C)C1]

To a dichloromethane (1.5 mL) solution of 1-((S)-4-(6-(5-methoxy-2-methylbenzoyl)-2-(((S)-1-methylpyrrolidin-2-yl)methoxy)-6,7-dihydro-5H-pyrrolo[3,4-d]pyrimidin-4-yl)-3-methylpiperazin-1-yl)prop-2-en-1-one (4, 21 mg, 0.039 mmol) was added boron tribromide (0.22 mL, 0.22 mmol, 1M in dichloromethane) at 0° C. The mixture was stirred for 5 hours at room temperature. The mixture was diluted with methanol and aqueous sodium bicarbonate. Organic materials were extracted with ethyl acetate twice and combined extracts were washed with brine, dried over anhydrous magnesium sulfate, and concentrated in vacuo after filtration. Purification by preparative HPLC (5-95% acetonitrile in water+0.1% formic acid) gave 1-((S)-4-(6-(5-hydroxy-2-methylbenzoyl)-2-(((S)-1-methylpyrrolidin-2-yl)methoxy)-6,7-dihydro-5H-pyrrolo[3,4-d]pyrimidin-4-yl)-3-methylpiperazin-1-yl)prop-2-en-1-one (1-23) as beige solid as the formic acid salt. Yield=3.5 mg (16%). MS 521.3 [M+H]⁺.

### Representative Procedure F. Synthesis of 1-26.

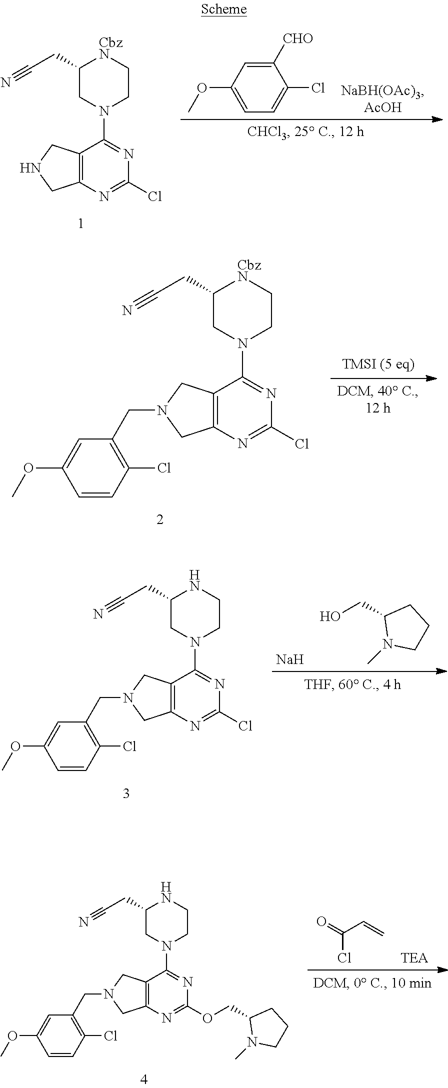

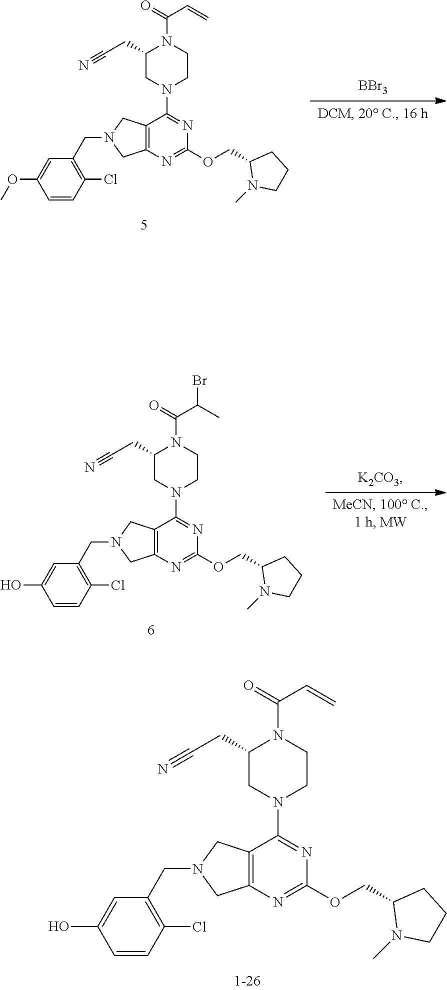

### Step 1: Synthesis of benzyl (S)-4-(2-chloro-6-(2-chloro-5-methoxybenzyl)-6,7-dihydro-5H-pyrrolo[3,4-d] pyrimidin-4-yl)-2-(cyanomethyl) piperazine-1-carboxylate (2)

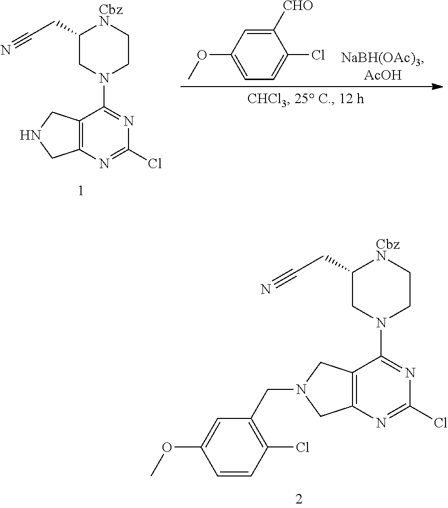

A mixture of benzyl (S)-4-(2-chloro-6,7-dihydro-5H-pyrrolo[3,4-d]pyrimidin-4-yl)-2-(cyanomethyl)piperazine-1-carboxylate (1) (750 mg, 1.82 mmol, 1 equiv., for the procedure of 1, please refer to the procedure of compound 1-7), 2-chloro-5-methoxybenzaldehyde (464 mg, 2.73 mmol, 1.5 equiv.), sodium triacetoxyborohydride (978 mg, 4.55 mmol, 2.5 equiv.) and acetic acid (0.2 mL) in chloroform (15 mL) was stirred at 25° C. for 12 h. The reaction mixture was concentrated in vacuo and the residue was extracted with dichloromethane (20 mL×3) and water (10 mL). The organic phase was combined, and dried over sodium sulfate, filtered. The filtrate was evaporated to dryness and the residue was purified by chromatography silica gel (dichloromethane/methanol=80:1) to give benzyl (S)-4-(2-chloro-6-(2-chloro-5-methoxybenzyl)-6,7-dihydro-5H-pyrrolo[3,4-d]pyrimidin-4-yl)-2-(cyanomethyl)piperazine-1-carboxylate (2) (367 mg, 35.6% yield) as yellow solid. ESI-MS m/z=567.2 [M+H]⁺. Calculated MW: 566.16.

### Step 2: Synthesis of (S)-2-(4-(2-chloro-6-(2-chloro-5-methoxybenzyl)-6,7-dihydro-5H-pyrrolo[3,4-d]pyrimidin-4-yl)piperazin-2-yl)acetonitrile (3)

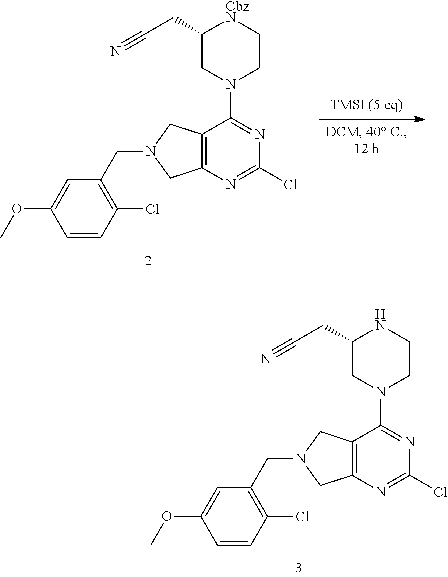 [SMILES: COc1ccc(Cl)c(CN2Cc3nc(Cl)nc(N4CCN(C(=O)OCc5ccccc5)[C@@H](CC#N)C4)c3C2)c1 ; "DCM, 40° C.,12 h" ; "TMSI (5 eq)" ; COc1ccc(Cl)c(CN2Cc3nc(Cl)nc(N4CCN[C@@H](CC#N)C4)c3C2)c1]

To a mixture of benzyl (S)-4-(2-chloro-6-(2-chloro-5-methoxybenzyl)-6,7-dihydro-5H-pyrrolo[3,4-d]pyrimidin-4-yl)-2-(cyanomethyl)piperazine-1-carboxylate (2) (367 mg, 0.647 mmol, 1 equiv.) in dichloromethane (5 mL) was added trimethylsilyl iodide (647 mg, 3.235 mmol, 5 equiv.), and the reaction mixture was stirred at 40° C. for 12 h. The mixture was quenched with methanol (2 mL), and concentrated under reduced pressure to give residue, which was purified by chromatography silica gel (dichloromethane/methanol=80:1) to give (S)-2-(4-(2-chloro-6-(2-chloro-5-methoxybenzyl)-6,7-dihydro-5H-pyrrolo[3,4-d]pyrimidin-4-yl)piperazin-2-yl)acetonitrile (3) (170 mg, 60.7% yield) as white solid. ESI-MS m/z=433.1[M+H]⁺. Calculated MW: 432.12.

### Step 3: Synthesis of 2-((S)-4-(6-(2-chloro-5-methoxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (4)

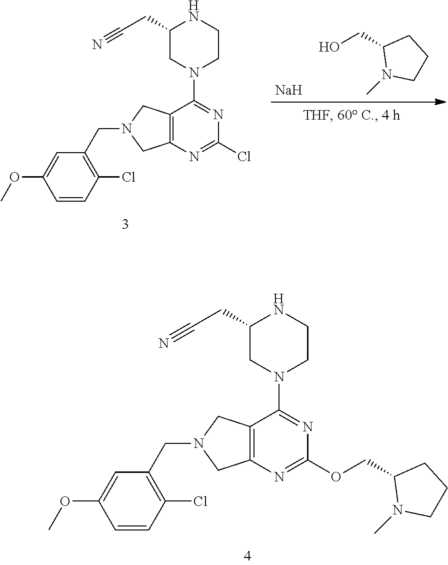

To a mixture of (S)-(1-methylpyrrolidin-2-yl)methanol (225.4 mg, 1.96 mmol, 5 equiv.) in tetrahydrofuran (10 mL) was added 60% sodium hydride (79 mg, 1.96 mmol, 5 equiv.) and the reaction mixture was stirred at 25° C. for 10 min. (S)-2-(4-(2-chloro-6-(2-chloro-5-methoxybenzyl)-6,7-dihydro-5H-pyrrolo[3,4-d]pyrimidin-4-yl)piperazin-2-yl)acetonitrile (3) (170 mg, 0.394 mmol, 1 equiv.) was added, and the reaction was stirred at 60° C. for 4 h. The reaction mixture was poured into ice-water (30 mL) slowly, and extracted with dichloromethane (15 mL×3). The organic phase was combined, dried over sodium sulfate, filtered and concentrated in vacuo. The residue was purified by silica gel chromatography (dichloromethane/methanol=10:1) to give 2-((S)-4-(6-(2-chloro-5-methoxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (4) (123 mg, 61.2% yield) as yellow solid. ESI-MS m/z=512.3 [M+H]⁺. Calculated MW: 511.25.

### Step 4: Synthesis of 2-((S)-1-acryloyl-4-(6-(2-chloro-5-methoxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (5)

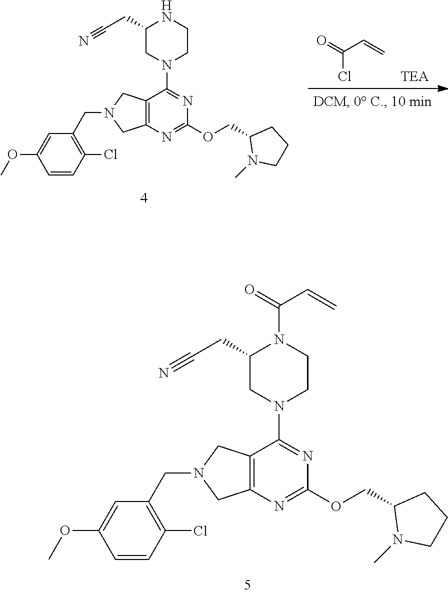

To a mixture 2-((S)-4-(6-(2-chloro-5-methoxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (4) (123 mg, 0.24 mmol, 1 equiv.) and triethylamine (121 mg, 1.2 mmol, 5 equiv.) in dichloromethane (10 mL) was added dropwise of acryloyl chloride (162 mg, 1.8 mmol, 1.5 equiv.) at 0° C. After addition, the resulting mixture was stirred under nitrogen at 0° C. for 10 minutes. The reaction was quenched with water (20 mL), extracted with dichloromethane (20 mL×3). The organic phase was combined and dried over sodium sulfate, filtered. The filtrate was evaporated to dryness and the residue was purified by chromatography silica gel (dichloromethane/methanol=10:1) to give 2-((S)-1-acryloyl-4-(6-(2-chloro-5-methoxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (5) (72 mg, 53.3% yield) as white solid. ESI-MS m/z=566.3 [M+H]⁺. Calculated MW: 565.26

### Step 5: Synthesis of 2-((2S)-1-(2-bromopropanoyl)-4-(6-(2-chloro-5-hydroxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (6)

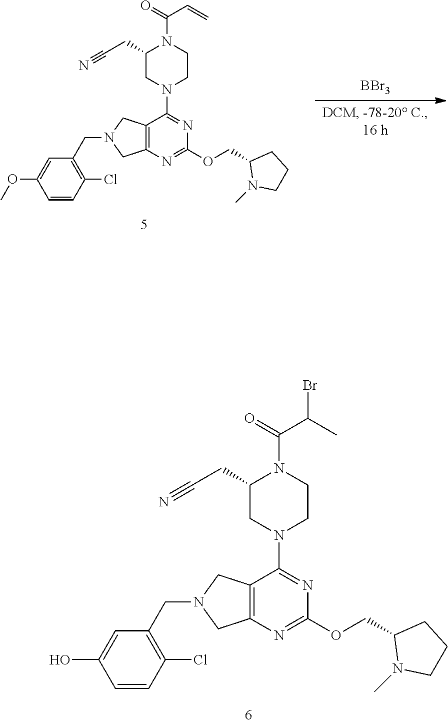 [SMILES: C=CC(=O)N1CCN(c2nc(OC[C@@H]3CCCN3C)nc3c2CN(Cc2cc(OC)ccc2Cl)C3)C[C@@H]1CC#N ; "BBr3" ; "DCM, -78-20° C.,16 h" ; CC(Br)C(=O)N1CCN(c2nc(OC[C@@H]3CCCN3C)nc3c2CN(Cc2cc(O)ccc2Cl)C3)C[C@@H]1CC#N]

To a mixture of 2-((S)-1-acryloyl-4-(6-(2-chloro-5-methoxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (5) (72 mg, 0.127 mmol, 1 equiv.) in dichloromethane (10 mL) was added BBr₃ (324 mg, 1.27 mmol, 10 equiv.) at −78° C. slowly, and the reaction mixture was stirred at −78 to 20° C. for 16 h. The reaction was quenched with saturated NaHCO₃ (20 mL), extracted with dichloromethane (20 mL×3). The organic phase was combined, and dried over sodium sulfate, filtered. The filtrate was evaporated to dryness and the residue was purified by chromatography silica gel (dichloromethane/methanol=10:1) to give 2-((2S)-1-(2-bromopropanoyl)-4-(6-(2-chloro-5-hydroxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (6) (72 mg, 90% yield) as white solid. ESI-MS m/z=632.1 [M+H]⁺. Calculated MW: 631.17

### Step 6: Synthesis of 2-((S)-1-acryloyl-4-(6-(2-chloro-5-hydroxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (1-26)

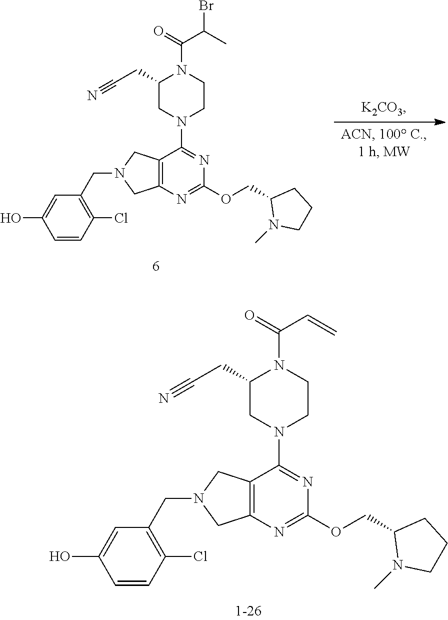 [SMILES: CC(Br)C(=O)N1CCN(c2nc(OC[C@@H]3CCCN3C)nc3c2CN(Cc2cc(O)ccc2Cl)C3)C[C@@H]1CC#N ; "ACN, 100° C.,1 h, MW" ; "K2CO3," ; C=CC(=O)N1CCN(c2nc(OC[C@@H]3CCCN3C)nc3c2CN(Cc2cc(O)ccc2Cl)C3)C[C@@H]1CC#N]

To a mixture of 2-((2S)-1-(2-bromopropanoyl)-4-(6-(2-chloro-5-hydroxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (6) (72 mg, 0.114 mmol, 1 equiv.) in acetonitrile (5 mL) was added K₂CO₃ (79 mg, 0.57 mmol, 5 equiv.), the mixture was stirred at 100° C. for 1 h under microwave irradiation. The reaction was cooled down to room temperature, filtered and the filtrate was concentrated in vacuo. The residue was purified by chromatography silica gel (dichloromethane/methanol=10:1) to give 2-((S)-1-acryloyl-4-(6-(2-chloro-5-hydroxybenzyl)-2-(((S)-1-methylpyrrolidin-2-yl)methoxy)-6,7-dihydro-5H-pyrrolo[3,4-d]pyrimidin-4-yl)piperazin-2-yl)acetonitrile (1-26) (18 mg, 28.5% yield) as white solid. ESI-MS m/z=552.3 [M+H]⁺. Calculated MW: 551.24.

### Representative Procedure G. Synthesis of 1-37.

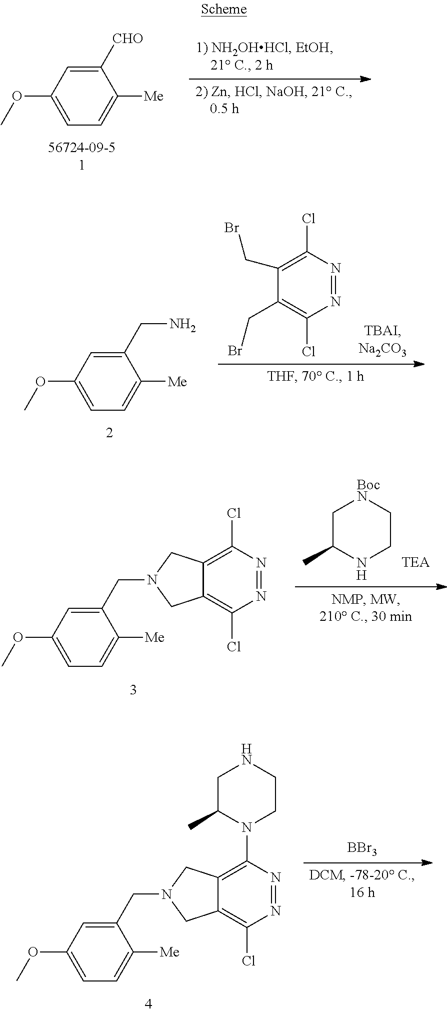

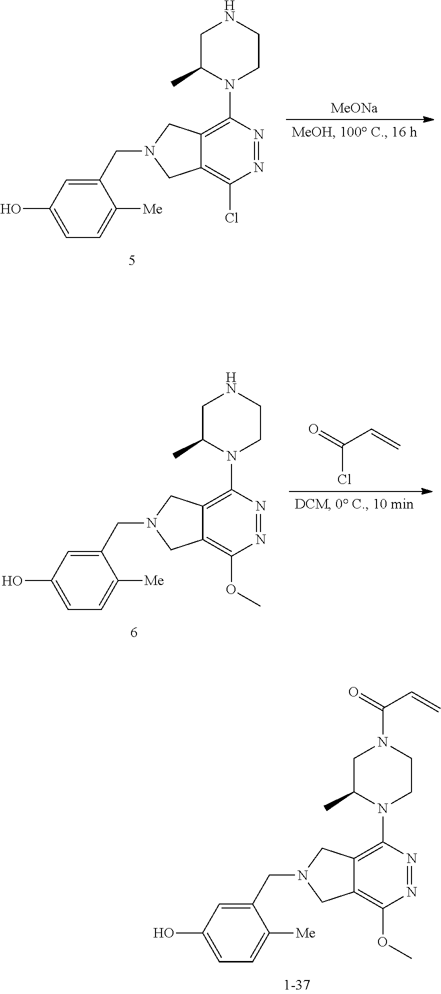

### Step 1: Synthesis of (5-methoxy-2-methylphenyl) methanamine (2)

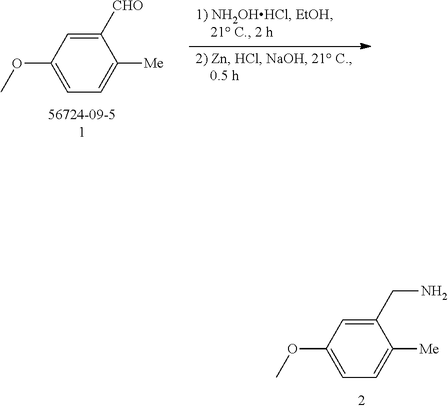

A solution of 5-methoxy-2-methylbenzaldehyde (1) (800 mg, 6.7 mmol, 1 eq., CAS:56724-09-5) and hydroxylammonium chloride (444 mg, 8 mmol, 1.2 eq.) in ethanol (10 ml) was stirred at 21° C. for 2 h. Subsequently, hydrochloric acid (7.8 mL, 26 mmol, 4 eq.) and zinc dust (871 mg, 16.6 mmol, 5 eq.) were slowly added to the solution and the mixture was stirred at 21° C. for 15 min. A solution of ammonia (30%, 14 mL) and sodium hydroxide (6 M, 5 mL) was added dropwise to the resulting slurry and the mixture was stirred at 21° C. for 30 min. Then, the resultant solution was extracted with dichloromethane (20 mL×3), the organic layers were combined, dried over anhydrous sodium sulfate and filtered. The solvent was removed under vacuum to afford (5-methoxy-2-methylphenyl)methanamine (2) (272 mg, 30.1% yield) as white oil without further purification. ESI-MS m/z=152.1 [M+H]⁺. Calculated MW: 151.10.

### Step 2: Synthesis of 4,7-dichloro-2-[(5-methoxy-2-methylphenyl)methyl]-1,3-dihydroisoindole (3)

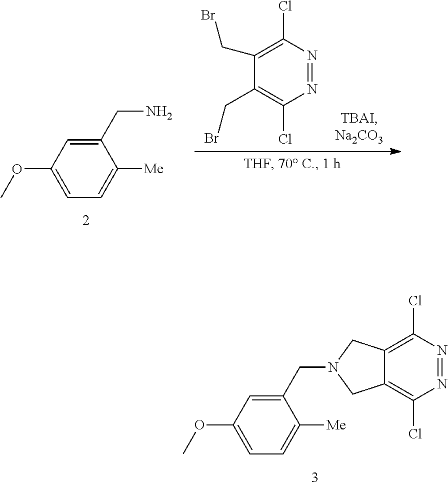

To a suspension 4,5-bis(bromomethyl)-3,6-dichloropyridazine (2) (1000 mg, 3 mmol, 1 eq.) in anhydrous tetrahydrofuran (100 mL) was added sodium carbonate (950 mg, 9 mmol, 3 eq.) and TBAI (118 mg, 0.3 mmol, 0.1 eq.). Then to the reaction mixture was added dropwise a solution of (5-methoxy-2-methylphenyl)methanamine (450 mg, 3 mmol) in tetrahydrofuran (50 mL) over 2 h. The reaction mixture was stirred at 20° C. for 30 min and heated to 70° C. for 1 h and concentrated. The reaction was quenched by 1 N HCl (20 mL), extracted with dichloromethane (15 mL×3), the organic layers were combined, dried over anhydrous sodium sulfate, filtered and concentrated in vacuo. The mixture was purified by flash chromatography on silica gel (petroleum ether/ethyl acetate=5:1) to give 4,7-dichloro-2-[(5-methoxy-2-methylphenyl)methyl]-1,3-dihydroisoindole (3) (298 mg, 27.9% yield) as an orange solid. ESI-MS m/z=324.1 [M+H]⁺. Calculated MW: 323.06.

### Step 3: Synthesis of (S)-1-chloro-6-(5-methoxy-2-methylbenzyl)-4-(2-methylpiperazin-1-yl)-6,7-dihydro-5H-pyrrolo[3,4-d]pyridazine (4)

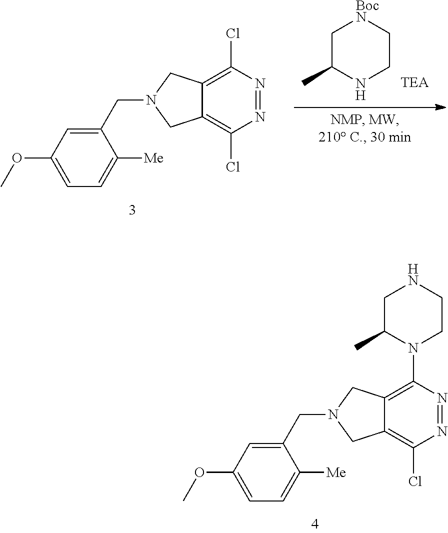

To a solution of 4,7-dichloro-2-[(5-methoxy-2-methylphenyl)methyl]-1,3-dihydroisoindole (3) (600 mg, 1.9 mmol, 1 eq.) in N-methyl pyrrolidinone (12 mL) was added tert-butyl (3S)-3-methylpiperazine-1-carboxylate (1.5 g, 7.44 mmol, 4 eq.) and Et₃N (0.3 mL), the mixture was stirred at 20° C. for 20 min. Then the mixture was stirred at 210° C. under microwave for 30 min. The reaction was quenched by water (120 mL), and extracted by ethyl acetate (20 mL×3), then washed by brine (30 mL), the organic layers were combined, dried over anhydrous sodium sulfate, filtered and concentrated in vacuo. The residue was purified by silica gel flash chromatography (petroleum ether/ethyl acetate=3:1) to give (S)-1-chloro-6-(5-methoxy-2-methylbenzyl)-4-(2-methylpiperazin-1-yl)-6,7-dihydro-5H-pyrrolo[3,4-d]pyridazine (4) (181 mg, 17.8% yield) was obtained as orange oil. ESI-MS m/z=388.2 [M+H]⁺. Calculated MW: 387.18.

### Step 4: Synthesis of (S)-3-((1-chloro-4-(2-methylpiperazin-1-yl)-5,7-dihydro-6H-pyrrolo[3,4-d]pyridazin-6-yl)methyl)-4-methylphenol (5)

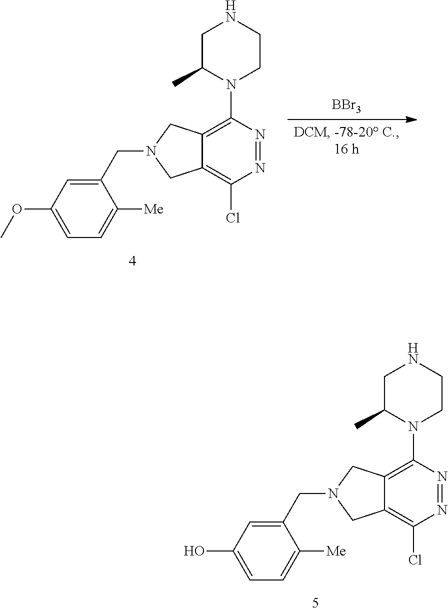 [SMILES: COc1ccc(C)c(CN2Cc3c(Cl)nnc(N4CCNC[C@@H]4C)c3C2)c1 ; "BBr3" ; "DCM, -78-20° C.,16 h" ; Cc1ccc(O)cc1CN1Cc2c(Cl)nnc(N3CCNC[C@@H]3C)c2C1]

To a solution of (2S)-1-{4-chloro-6-[(5-methoxy-2-methylphenyl)methyl]-5H,7H-pyrrolo[3,4-d]pyridazin-1-yl}-2-methylpiperazine (4) (0.27 g, 0.7 mmol, 1 eq.) in CH₂Cl₂ (20 mL) was added a solution of BBr₃ (1.7 g, 7 mmol, 10 eq.) in CH₂Cl₂ (5 mL) dropwise at −78° C. under nitrogen for 5 min. The reaction mixture was stirred at −78 to 25° C. for 16 h. The mixture was quenched with saturated NaHCO₃ and the pH adjusted to 7 followed by extraction with CH₂Cl₂ (20 mL×3). The organic layers were combined, dried over anhydrous sodium sulfate, filtered and concentrated in vacuo. The residue was purified by silica gel flash chromatography (dichloromethane/methanol=10/1) to give (S)-3-((1-chloro-4-(2-methylpiperazin-1-yl)-5,7-dihydro-6H-pyrrolo[3,4-d]pyridazin-6-yl)methyl)-4-methylphenol (5) (210 mg, 72.2 yield) as white solid. ESI-MS m/z=374.2 [M+H]⁺. Calculated MW: 373.17.

### Step 5: Synthesis of (S)-3-((1-chloro-4-(2-methylpiperazin-1-yl)-5,7-dihydro-6H-pyrrolo[3,4-d]pyridazin-6-yl)methyl)-4-methylphenol (6)

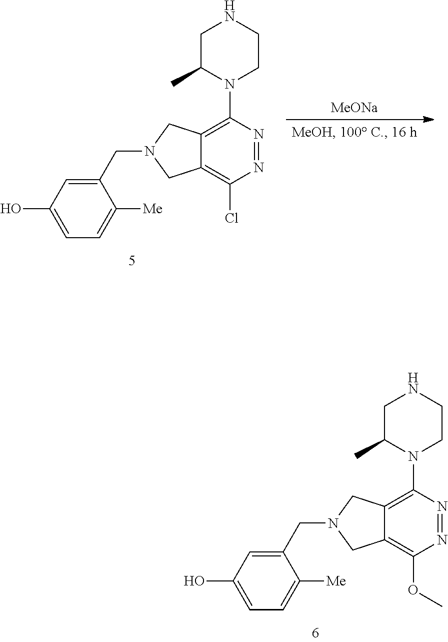 [SMILES: "MeONa" ; Cc1ccc(O)cc1CN1Cc2c(Cl)nnc(N3CCNC[C@@H]3C)c2C1 ; "MeOH, 100° C., 16 h" ; COc1nnc(N2CCNC[C@@H]2C)c2c1CN(Cc1cc(O)ccc1C)C2]

To a solution of 3-({1-chloro-4-[(2S)-2-methylpiperazin-1-yl]-5H,7H-pyrrolo[3,4-d]pyridazin-6-yl}methyl)-4-methylphenol (5) (114 mg, 0.31 mmol, 1 eq.) in methanol (5 mL) was added sodium methylate (486 mg, 9 mmol, 10 eq.), the mixture was stirred at 100° C. for 16 h. The reaction was quenched, filtered and concentrated in vacuo. The residue was directly purified by silica gel flash chromatography (dichloromethane/methanol=10/1) to give (S)-3-((1-chloro-4-(2-methylpiperazin-1-yl)-5,7-dihydro-6H-pyrrolo[3,4-d]pyridazin-6-yl)methyl)-4-methylphenol (6) (51 mg, 40.1% yield) as white solid. ESI-MS m/z=370.3 [M+H]⁺. Calculated MW: 369.22.

### Step 6: Synthesis of(S)-3-((1-chloro-4-(2-methylpiperazin-1-yl)-5,7-dihydro-6H-pyrrolo[3,4-d]pyridazin-6-yl)methyl)-4-methylphenol (1-37)

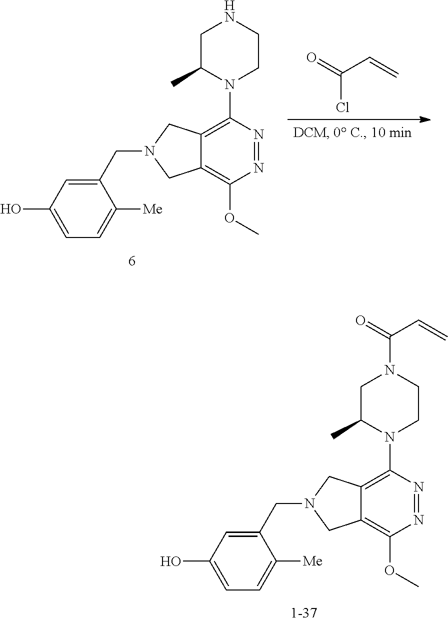

To a solution of 3-({1-methoxy-4-[(2S)-2-methylpiperazin-1-yl]-5H,7H-pyrrolo[3,4-d]pyridazin-6-yl}methyl)-4-methylphenol (6) (70 mg, 0.19 mmol, 1 eq.) and triethylamine (58 mg, 0.57 mmol, 3 eq.) in CH₂Cl₂ (2 mL) was added a solution of prop-2-enoyl chloride (19 mg, 0.2 mmol, 1.05 eq.) in CH₂Cl₂ (1 mL) at 0° C. The mixture was then stirred at 0° C. for 10 minutes. The mixture was quenched by water (15 mL), extracted by CH₂Cl₂ (20 mL×3), the organic layers were combined, dried over anhydrous sodium sulfate, filtered and concentrated in vacuo. The residue was purified by preparative-thin layer chromatography (dichloromethane/methanol=10/1) to give (S)-3-((1-chloro-4-(2-methylpiperazin-1-yl)-5,7-dihydro-6H-pyrrolo[3,4-d]pyridazin-6-yl)methyl)-4-methylphenol (1-37) (43 mg, 71.8% yield) as a white solid. ESI-MS m/z=424.2 [M+H]⁺. Calculated MW: 423.23.

### Representative Procedure H. Synthesis of 1-69.

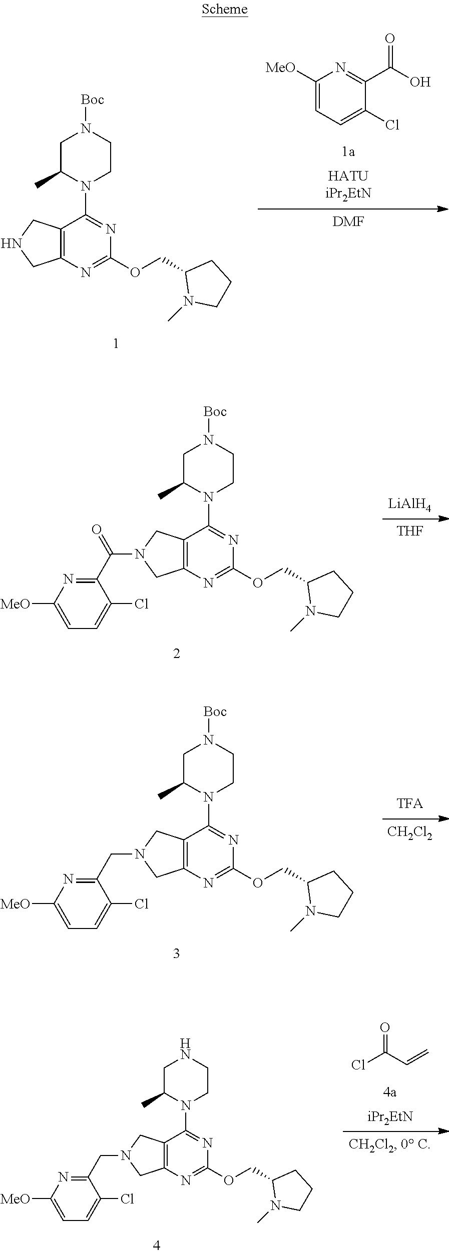

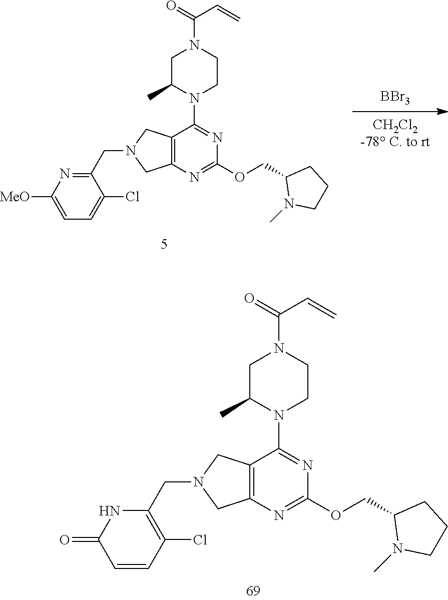 [SMILES: C=CC(=O)N1CCN(c2nc(OC[C@@H]3CCCN3C)nc3c2CN(Cc2nc(OC)ccc2Cl)C3)[C@@H](C)C1 ; "BBr3" ; "CH2Cl2-78° C. to rt" ; C=CC(=O)N1CCN(c2nc(OC[C@@H]3CCCN3C)nc3c2CN(Cc2[nH]c(=O)ccc2Cl)C3)[C@@H](C)C1]

### Step 1: Synthesis of tert-butyl (S)-4-(6-(3-chloro-6-methoxypicolinoyl)-2-(((S)-1-methylpyrrolidin-2-yl)methoxy)-6,7-dihydro-5H-pyrrolo[3,4-d]pyrimidin-4-yl)-3-methylpiperazine-1-carboxylate (2)

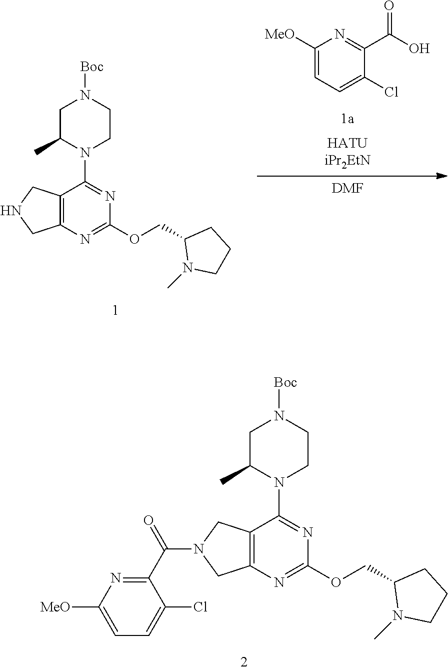

To a N,N-dimethylformamide (2 mL) solution of 3-chloro-6-methoxypicolinic acid (1a, 73 mg, 0.39 mmol) was added 1-[bis(dimethylamino)methylene]-1H-1,2,3-triazolo[4,5-b]pyridinium 3-oxide hexafluorophosphate (171 mg, 0.45 mmol), N,N-diisopropylethylamine (0.16 mL, 0.90 mmol), and tert-butyl (S)-3-methyl-4-(2-(((S)-1-methylpyrrolidin-2-yl)methoxy)-6,7-dihydro-5H-pyrrolo[3,4-d]pyrimidin-4-yl)piperazine-1-carboxylate (1, 129 mg, 0.30 mmol) at room temperature. The mixture was stirred for 3 h at room temperature. The mixture was diluted with ethyl acetate and washed with brine three times, dried over anhydrous magnesium sulfate, and concentrated in vacuo after filtration. Purification by silica gel column chromatography (0-10% methanol in dichloromethane) gave tert-butyl (S)-4-(6-(3-chloro-6-methoxypicolinoyl)-2-(((S)-1-methylpyrrolidin-2-yl)methoxy)-6,7-dihydro-5H-pyrrolo[3,4-d]pyrimidin-4-yl)-3-methylpiperazine-1-carboxylate (2) as yellow oil. Yield: 179 mg, 99%. MS: 602.2 [M+H]⁺.

### Step 2: Synthesis of tert-butyl (S)-4-(6-((3-chloro-6-methoxypyridin-2-yl)methyl)-2-(((S)-1-methylpyrrolidin-2-yl)methoxy)-6,7-dihydro-5H-pyrrolo[3,4-d]pyrimidin-4-yl)-3-methylpiperazine-1-carboxylate (3)

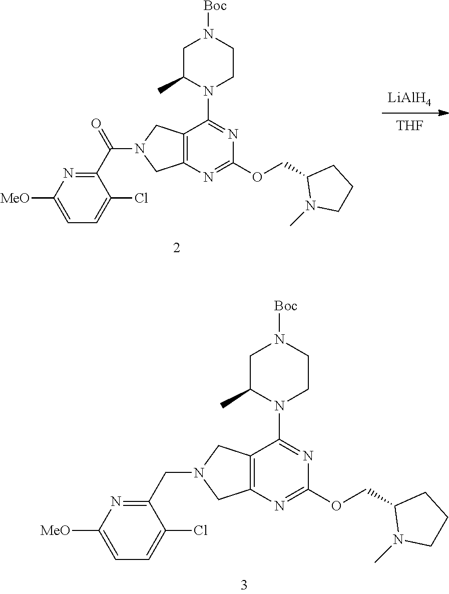 [SMILES: COc1ccc(Cl)c(C(=O)N2Cc3nc(OC[C@@H]4CCCN4C)nc(N4CCN(C(=O)OC(C)(C)C)C[C@@H]4C)c3C2)n1 ; "LiAlH4" ; "THF" ; COc1ccc(Cl)c(CN2Cc3nc(OC[C@@H]4CCCN4C)nc(N4CCN(C(=O)OC(C)(C)C)C[C@@H]4C)c3C2)n1]

To a tetrahydrofuran (2 mL) solution of tert-butyl (S)-4-(6-(3-chloro-6-methoxypicolinoyl)-2-(((S)-1-methylpyrrolidin-2-yl)methoxy)-6,7-dihydro-5H-pyrrolo[3,4-d]pyrimidin-4-yl)-3-methylpiperazine-1-carboxylate (2, 135 mg, 0.22 mmol) was added lithium aluminium hydride (0.34 mL, 0.34 mmol, 1M in tetrahydrofuran) at 0° C. The mixture was stirred for 1 h at room temperature. The mixture was diluted with diethyl ether. Water (0.05 mL), 2N sodium hydroxide (0.05 mL) and water (0.15 mL) were added to the mixture at 0° C. The mixture was stirred for 30 minutes at room temperature. Magnesium sulfate was added to the mixture. After stirring the mixture for 30 minutes, the mixture was filtered. The filtrate was concentrated in vacuo. Purification by silica gel column chromatography (0-10% methanol in dichloromethane) gave tert-butyl (S)-4-(6-((3-chloro-6-methoxypyridin-2-yl)methyl)-2-(((S)-1-methylpyrrolidin-2-yl)methoxy)-6,7-dihydro-5H-pyrrolo[3,4-d]pyrimidin-4-yl)-3-methylpiperazine-1-carboxylate (3) as yellow oil. Yield: 53 mg, 40%. MS: 588.3 [M+H]⁺.

### Step 3: Synthesis of 6-((3-chloro-6-methoxypyridin-2-yl)methyl)-4-((S)-2-methylpiperazin-1-yl)-2-(((S)-1-methylpyrrolidin-2-yl)methoxy)-6,7-dihydro-5H-pyrrolo[3,4-d]pyrimidine (4)

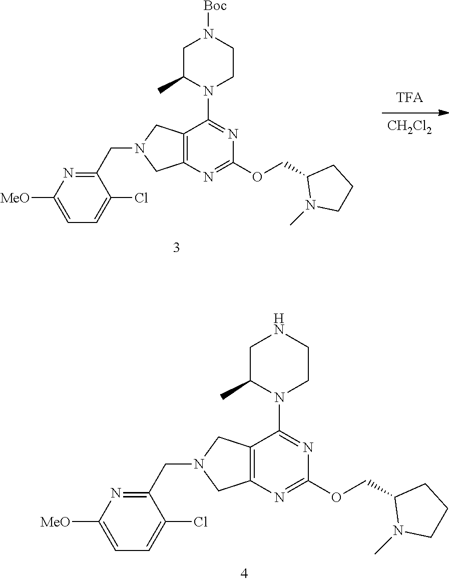 [SMILES: "TFA" ; COc1ccc(Cl)c(CN2Cc3nc(OC[C@@H]4CCCN4C)nc(N4CCN(C(=O)OC(C)(C)C)C[C@@H]4C)c3C2)n1 ; "CH2Cl2" ; COc1ccc(Cl)c(CN2Cc3nc(OC[C@@H]4CCCN4C)nc(N4CCNC[C@@H]4C)c3C2)n1]

To a dichloromethane (1 mL) solution of tert-butyl (S)-4-(6-((3-chloro-6-methoxypyridin-2-yl)methyl)-2-(((S)-1-methylpyrrolidin-2-yl)methoxy)-6,7-dihydro-5H-pyrrolo[3,4-d]pyrimidin-4-yl)-3-methylpiperazine-1-carboxylate (3, 64 mg, 0.11 mmol) was added trifluoroacetic acid (1 mL) at 0° C. After stirring the mixture for 1 h at room temperature, the mixture was concentrated in vacuo. The residue was loaded onto a SCX column. The column was eluted with methanol, followed by 2N ammonia in methanol. Basic fraction was concentrated in vacuo to give 6-((3-chloro-6-methoxypyridin-2-yl)methyl)-4-((S)-2-methylpiperazin-1-yl)-2-(((S)-1-methylpyrrolidin-2-yl)methoxy)-6,7-dihydro-5H-pyrrolo[3,4-d]pyrimidine (4) as red oil. Yield: 42 mg, 79%. MS: 488.3 [M+H]⁺.

### Step 4: Synthesis of 1-((S)-4-(6-((3-chloro-6-methoxypyridin-2-yl)methyl)-2-(((S)-1-methylpyrrolidin-2-yl)methoxy)-6,7-dihydro-5H-pyrrolo[3,4-d]pyrimidin-4-yl)-3-methylpiperazin-1-yl)prop-2-en-1-one (5)

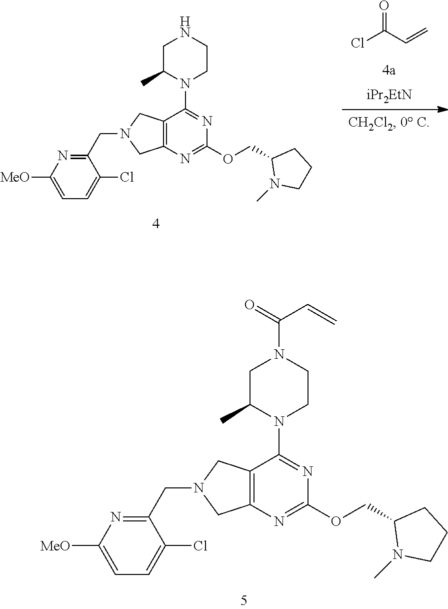

To a dichloromethane (1 mL) solution of 6-((3-chloro-6-methoxypyridin-2-yl)methyl)-4-((S)-2-methylpiperazin-1-yl)-2-(((S)-1-methylpyrrolidin-2-yl)methoxy)-6,7-dihydro-5H-pyrrolo[3,4-d]pyrimidine (4, 42 mg, 0.090 mmol) was added N,N-diisopropylethylamine (0.34 mL, 0.17 mmol, 0.5M in dichloromethane) and acryloyl chloride (4a, 0.19 mL, 0.090 mmol, 0.5M in dichloromethane) at 0° C. After stirring the mixture for 30 minutes at 0° C., the mixture was concentrated in vacuo. Purification by preparative HPLC (Cis column, 5-95% acetonitrile in water+0.1% formic acid) gave 1-((S)-4-(6-((3-chloro-6-methoxypyridin-2-yl)methyl)-2-(((S)-1-methylpyrrolidin-2-yl)methoxy)-6,7-dihydro-5H-pyrrolo[3,4-d]pyrimidin-4-yl)-3-methylpiperazin-1-yl)prop-2-en-1-one (5) as pink solid as formic acid salt. Yield 25 mg, 49%. MS 542.3 [M+H]⁺.

### Step 5: Synthesis of 6-((4-((S)-4-acryloyl-2-methylpiperazin-1-yl)-2-(((S)-1-methylpyrrolidin-2-yl)methoxy)-5,7-dihydro-6H-pyrrolo[3,4-d]pyrimidin-6-yl)methyl)-5-chloropyridin-2(1H)-one (69)

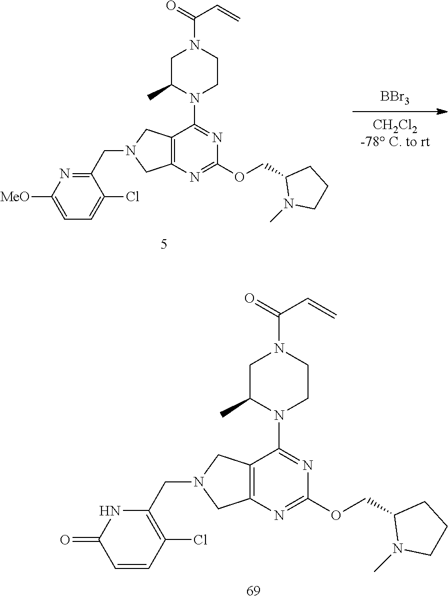 [SMILES: C=CC(=O)N1CCN(c2nc(OC[C@@H]3CCCN3C)nc3c2CN(Cc2nc(OC)ccc2Cl)C3)[C@@H](C)C1 ; "BBr3" ; "CH2Cl2-78° C. to rt" ; C=CC(=O)N1CCN(c2nc(OC[C@@H]3CCCN3C)nc3c2CN(Cc2[nH]c(=O)ccc2Cl)C3)[C@@H](C)C1]

To a dichloromethane (1.5 mL) solution of 1-((S)-4-(6-((3-chloro-6-methoxypyridin-2-yl)methyl)-2-(((S)-1-methylpyrrolidin-2-yl)methoxy)-6,7-dihydro-5H-pyrrolo[3,4-d]pyrimidin-4-yl)-3-methylpiperazin-1-yl)prop-2-en-1-one (5, 20 mg, 0.040 mmol) was added boron tribromide (0.11 mL, 0.11 mmol, 1M in dichloromethane) at −78° C. The mixture was stirred for 3 h at −78 to 0° C. Further boron tribromide (0.11 mL, 0.11 mmol, 1M in dichloromethane) was added to the mixture at 0° C. The mixture was stirred overnight at 0° C. to room temperature. The mixture was quenched with 2N sodium hydroxide and stirred for 15 minutes at 0° C. The mixture was extracted with 10% methanol in dichloromethane twice, dried over anhydrous magnesium sulfate, and concentrated in vacuo. LCMS indicated that product was mainly in water layer. The water layer was concentrated in vacuo. The residual solution was purified by preparative HPLC (5-95% acetonitrile in water+0.1% formic acid) to give 6-((4-((S)-4-acryloyl-2-methylpiperazin-1-yl)-2-(((S)-1-methylpyrrolidin-2-yl)methoxy)-5,7-dihydro-6H-pyrrolo[3,4-d]pyrimidin-6-yl)methyl)-5-chloropyridin-2(1H)-one (69) as beige solid as formic acid salt. Yield 1.5 mg, 7%. MS 528.3 [M+H]⁺.

### Representative Procedure L Synthesis of 1-126.

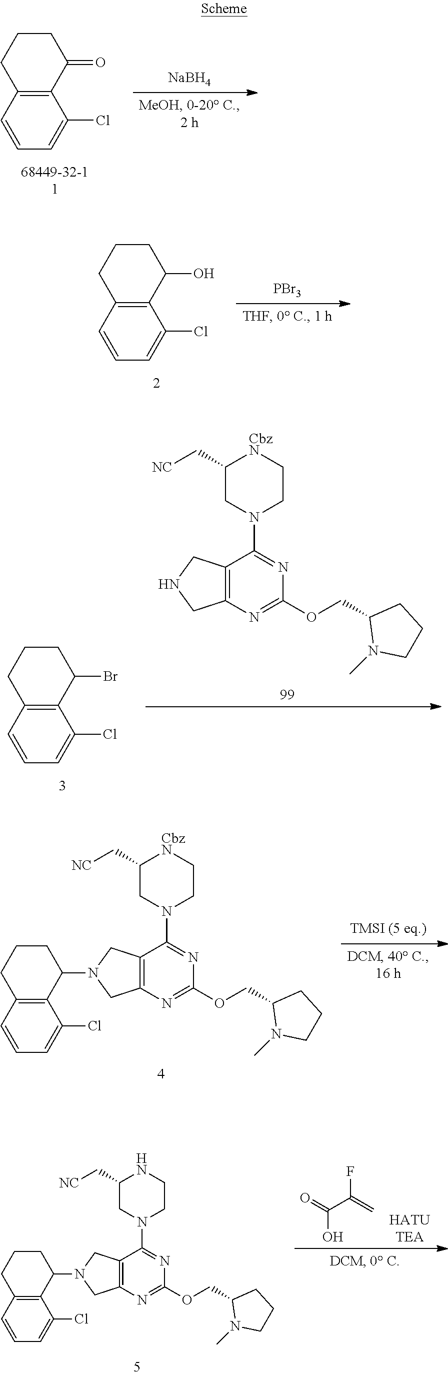

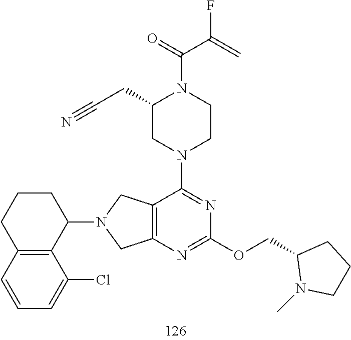 [SMILES: C=C(F)C(=O)N1CCN(c2nc(OC[C@@H]3CCCN3C)nc3c2CN(C2CCCc4cccc(Cl)c42)C3)C[C@@H]1CC#N]

### Step 1: Synthesis of 8-chloro-1,2,3,4-tetrahydronaphthalen-1-ol (2)

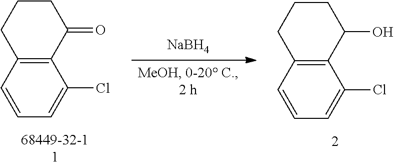 [SMILES: O=C1CCCc2cccc(Cl)c21 ; "MeOH, 0-20° C.,2 h" ; "NaBH4" ; OC1CCCc2cccc(Cl)c21]

To a solution of 8-chloro-3,4-dihydro-2H-naphthalen-1-one (1) (1000 mg, 5.6 mmol, 1 equiv.) in methanol (3 mL) was added sodium borohydride (425 mg, 11.2 mmol, 2 equiv.). The reaction mixture was stirred at 0-20° C. for 2 h. To the reaction mixture was added water (20 mL), extracted with ethyl acetate (10 mL×3). The organic layers were combined, dried over anhydrous sodium sulfate, filtered and concentrated in vacuo. The residue was purified by preparative-thin layer chromatography (petroleum ether/ethyl acetate=10:1) to give 8-chloro-1,2,3,4-tetrahydronaphthalen-1-ol (2) (820 mg, 77.9% yield) as a white solid. ESI-MS m/z=165.1 [M−OH]⁺. Calculated MW: 182.05

### Step 2: Synthesis of 1-bromo-8-chloro-1,2,3,4-tetrahydronaphthalene (3)

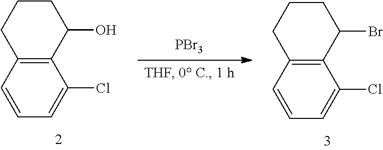 [SMILES: OC1CCCc2cccc(Cl)c21 ; "PBr3" ; "THF, 0° C., 1 h" ; Clc1cccc2c1C(Br)CCC2]

To a solution of 8-chloro-1,2,3,4-tetrahydronaphthalen-1-ol (2) (400 mg, 2.19 mmol, 1 equiv.) in tetrahydrofuran (4 mL) was added PBr₃ (710 mg, 2.63 mmol, 1.2 equiv.). The reaction mixture was stirred at 0° C. for 1 h. To the reaction mixture was added water (10 mL), extracted with ethyl acetate (10 mL×3). The organic was combined, dried over anhydrous sodium sulfate, filtered and concentrated in vacuo and the residue was purified by preparative-thin layer chromatography (petroleum ether/ethyl acetate=10:1) to give 1-bromo-8-chloro-1,2,3,4-tetrahydronaphthalene (3) (320 mg, 54.1% yield) as a colorless oil. ¹H NMR (400 MHz, CDCl3) δ 7.25-7.19 (m, 1H), 7.15 (t, J=7.8 Hz, 1H), 7.08-6.98 (m, 1H), 5.69 (s, 1H), 3.09-2.82 (m, 2H), 2.52-2.26 (m, 2H), 2.12-1.88 (m, 2H).

### Step 3: Synthesis of benzyl (2S)-4-[6-(8-chloro-1,2,3,4-tetrahydronaphthalen-1-yl)-2-{[(2S)-1-methylpyrrolidin-2-yl]methoxy}-5H,7H-pyrrolo[3,4-d]pyrimidin-4-yl]-2-(cyanomethyl)piperazine-1-carboxylate (4)

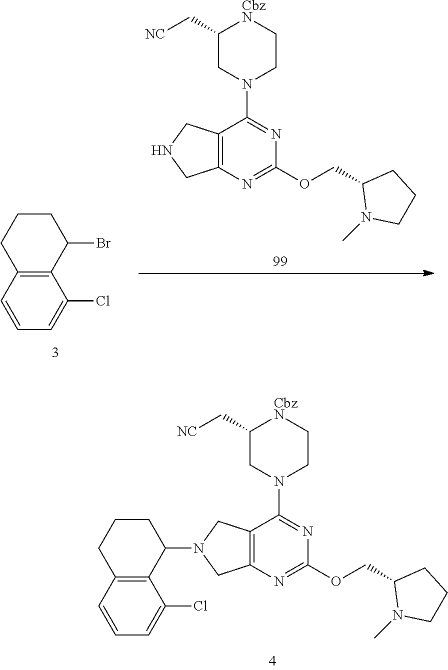 [SMILES: Clc1cccc2c1C(Br)CCC2 ; CN1CCC[C@H]1COc1nc2c(c(N3CCN(C(=O)OCc4ccccc4)[C@@H](CC#N)C3)n1)CNC2 ; "99" ; CN1CCC[C@H]1COc1nc2c(c(N3CCN(C(=O)OCc4ccccc4)[C@@H](CC#N)C3)n1)CN(C1CCCc3cccc(Cl)c31)C2]

To a solution of benzyl (2S)-2-(cyanomethyl)-4-(2-{[(2S)-1-methylpyrrolidin-2-yl]methoxy}-5H,6H,7H-pyrrolo[3,4-d]pyrimidin-4-yl)piperazine-1-carboxylate (99) (400 mg, 0.82 mmol, 1 equiv., for the procedure of 99, please refer to the procedure of compound 1-9) in tetrahydrofuran (5 mL) was added N,N-Diisopropylethylamine (210 mg, 1.64 mmol, 2 equiv.) and 1-bromo-8-chloro-1,2,3,4-tetrahydronaphthalene (3) (400 mg, 1.64 mmol, 2 equiv.). The reaction mixture was stirred at 60° C. for 15 h. To the reaction mixture was added water (10 mL), extracted with dichloromethane (10 mL×3). The organic layers were combined, dried over anhydrous sodium sulfate, filtered and concentrated in vacuo. The residue was purified by preparative-thin layer chromatography (dichloromethane/methanol=10:1) to give benzyl (2S)-4-[6-(8-chloro-1,2,3,4-tetrahydronaphthalen-1-yl)-2-{[(2S)-1-methylpyrrolidin-2-yl]methoxy}-5H,7H-pyrrolo[3,4-d]pyrimidin-4-yl]-2 (cyanomethyl)piperazine-1-carboxylate (4) (183 mg, 28.93% yield) as yellow oil. ESI-MS m/z=656.3[M+H]⁺. Calculated MW: 655.30

### Step 4: Synthesis of 2-[(2S)-4-[6-(2-chloro-5-methoxyphenyl)-2-{[(2S)-1-methylpyrrolidin-2-yl]methoxy}-5H,7H-pyrrolo[3,4-d]pyrimidin-4-yl]piperazin-2-yl]acetonitrile (5)

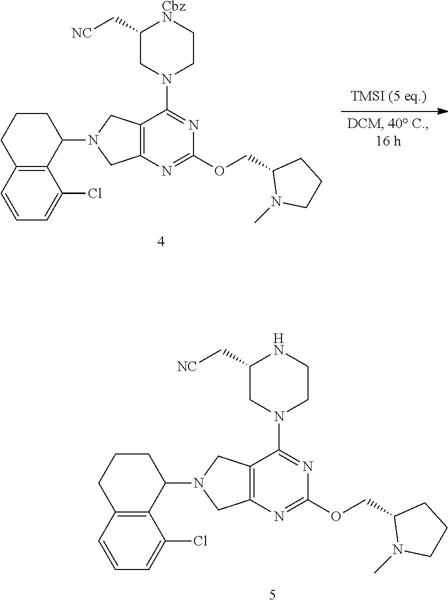 [SMILES: CN1CCC[C@H]1COc1nc2c(c(N3CCN(C(=O)OCc4ccccc4)[C@@H](CC#N)C3)n1)CN(C1CCCc3cccc(Cl)c31)C2 ; "DCM, 40° C.,16 h" ; "TMSI (5 eq.)" ; CN1CCC[C@H]1COc1nc2c(c(N3CCN[C@@H](CC#N)C3)n1)CN(C1CCCc3cccc(Cl)c31)C2]

To a solution of benzyl (2S)-4-[6-(2-chloro-5-methoxyphenyl)-2-{[(2S)-1-methylpyrrolidin-2-yl]methoxy}-5H,7H-pyrrolo[3,4-d]pyrimidin-4-yl]-2-(cyanomethyl)piperazine-1-carboxylate (4) (200 mg, 0.32 mmol, 1 equiv.) in dichloromethane (3 mL) was added trimethylsilyl iodide (360 mg, 2.56 mmol, 8 equiv.). The reaction mixture was stirred at 40° C. for 16 h. To the reaction mixture was added methanol (1 mL). The mixture was concentrated in vacuo and the residue was purified by preparative-thin layer chromatography (dichloromethane/methanol=10:1) to give 2-[(2S)-4-[6-(2-chloro-5-methoxyphenyl)-2-{[(2S)-1-methylpyrrolidin-2-yl]methoxy}-5H,7H-pyrrolo[3,4-d]pyrimidin-4-yl]piperazin-2-yl]acetonitrile (5) (67 mg, 71.5% yield) as yellow oil. ESI-MS m/z=522.3[M+H]⁺. Calculated MW: 521.27.

### Step 5: Synthesis of 2-[(2S)-4-[6-(8-chloro-1,2,3,4-tetrahydronaphthalen-1-yl)-2-{[(2S)-1-methylpyrrolidin-2-yl]methoxy}-5H,7H-pyrrolo[3,4-d]pyrimidin-4-yl]-1-(2-fluoroprop-2-enoyl)piperazin-2-yl]acetonitrile (126)

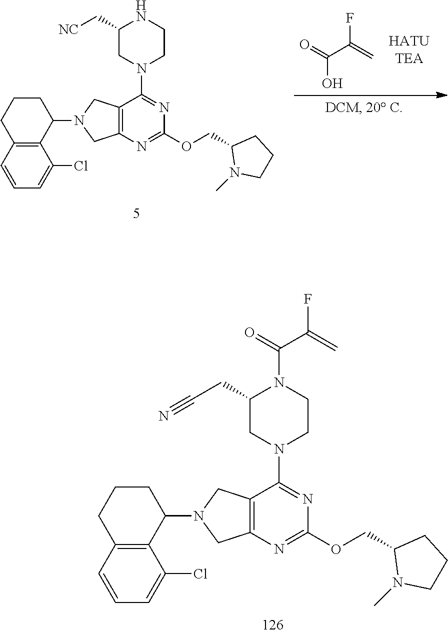

To a solution of 2-fluoroprop-2-enoic acid (26 mg, 0.29 mmol, 1.5 equiv.) in dimethylformamide (2 mL) was added triethylamine (60 mg, 0.59 mmol, 3.0 equiv.) and 1-[Bis(dimethylamino)methylene]-1H-1,2,3-triazolo[4,5-b]pyridinium 3-oxide hexafluorophosphate (HATU) (150 mg, 0.39 mmol, 2.0 equiv.). The reaction mixture was stirred at 20° C. for 10 min. Then to the solution was added 2-[(2S)-4-[6-(8-chloro-1,2,3,4-tetrahydronaphthalen-1-yl)-2-{[(2S)-1-methylpyrrolidin-2-yl]methoxy}-5H,7H-pyrrolo[3,4-d]pyrimidin-4-yl]piperazin-2-yl]acetonitrile (5) (100 mg, 0.19 mmol, 1 equiv.). The reaction mixture was stirred at 20° C. for 20 min. To the reaction mixture was added water (20 mL), extracted with ethyl acetate (10 mL×3). The organic layers were combined, dried over anhydrous sodium sulfate, filtered and concentrated in vacuo. The residue was purified by preparative-thin layer chromatography (dichloromethane/methanol=10:1) to give 2-[(2S)-4-[6-(8-chloro-1,2,3,4-tetrahydronaphthalen-1-yl)-2-{[(2S)-1-methylpyrrolidin-2-yl]methoxy}-5H,7H-pyrrolo[3,4-d]pyrimidin-4-yl]-1-(2-fluoroprop-2-enoyl)piperazin-2-yl]acetonitrile (126) (41 mg, 33.99% yield) as white solid. ESI-MS m/z=576.3[M+H]⁺. Calculated MW: 575.28.

### Representative Procedure J. Synthesis of 1-144.

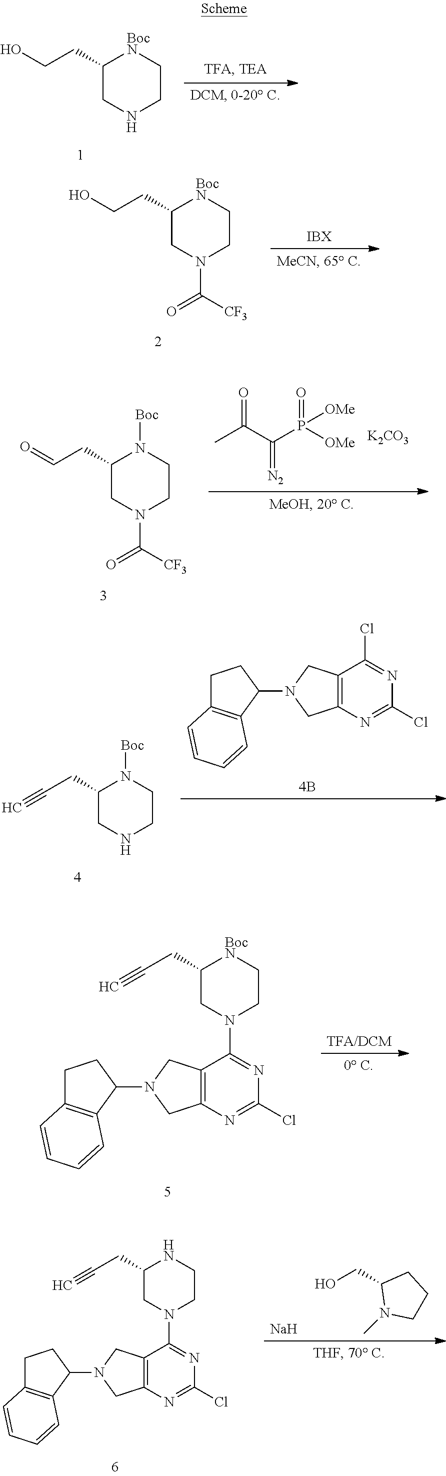

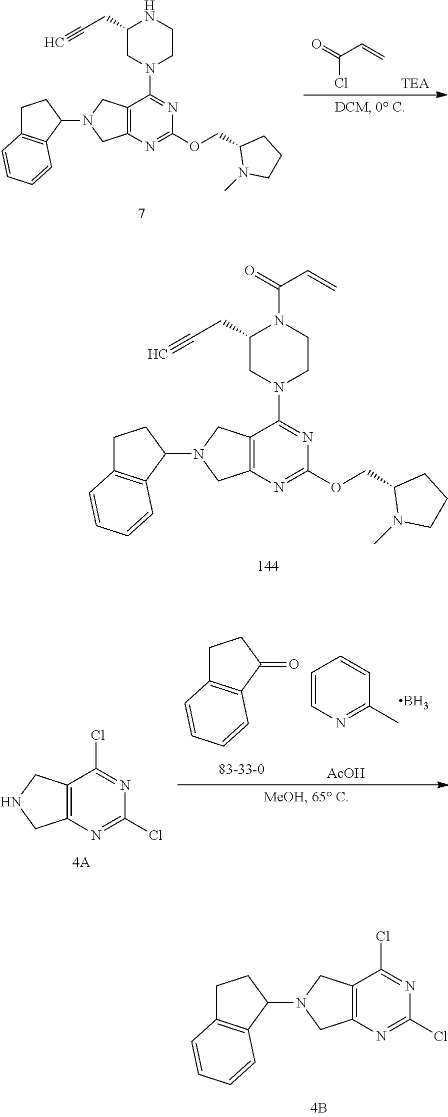

### Step 1: Synthesis of tert-butyl (S)-2-(2-hydroxyethyl)-4-(2,2,2-trifluoroacetyl)piperazine-1-carboxylate (2)

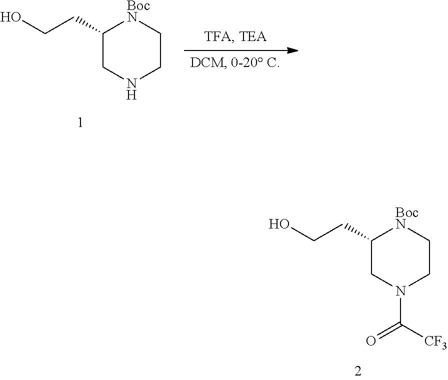 [SMILES: CC(C)(C)OC(=O)N1CCNC[C@@H]1CCO ; "DCM, 0-20° C." ; "TFA, TEA" ; CC(C)(C)OC(=O)N1CCN(C(=O)C(F)(F)F)C[C@@H]1CCO]

To a solution of tert-butyl (2S)-2-(2-hydroxyethyl)piperazine-1-carboxylate (1) (2.00 g, 8.7 mmol, 1.0 equiv.) and triethylamine (1.76 g, 17.4 mmol, 2.0 equiv.) in anhydrous dichloromethane (30 mL) was added trifluoroacetic acid (1.83 g, 8.7 mmol) dropwise at 0° C. The reaction solution was stirred at 0-20° C. for 16 h. The solution was quenched with water (20 mL), extracted with ethyl acetate (20 mL*3). The organic layers were combined, dried over sodium sulfate, filtered, and concentrated in vacuo. The residue was purified by silica gel column chromatography (petroleum ether/ethyl acetate=1:1) to give tert-butyl (S)-2-(2-hydroxyethyl)-4-(2,2,2-trifluoroacetyl)piperazine-1-carboxylate (2) (2.06 g, 66% yield) as a yellow oil. ESI-MS m/z=349.2[M+Na]⁺. Calculated MW: 326.15

### Step 2: Synthesis of tert-butyl (S)-2-(2-oxoethyl)-4-(2,2,2-trifluoroacetyl)piperazine-1-carboxylate (3)

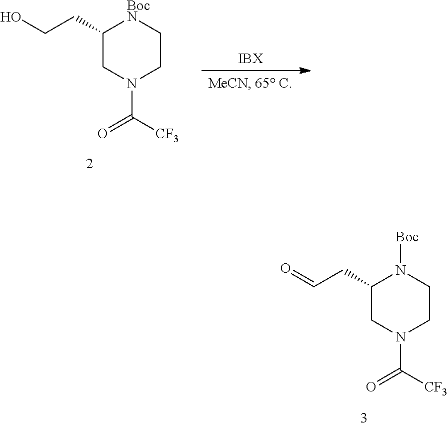

To a mixture of tert-butyl (2S)-2-(2-hydroxyethyl)-4-(2,2,2-trifluoroacetyl)piperazine-1-carboxylate (2) (2.00 g, 6.1 mmol, 1.0 equiv.) in acetonitrile (25 mL) was added 2-iodoxybenzoic acid (IBX) (8.54 g, 30.5 mol, 5.0 equiv.). The reaction solution was stirred at 65° C. for 1 h. The reaction solution was filtered, and the filtrate was concentrated in vacuo. The residue was diluted with ethyl acetate (30 mL), washed with water (20 mL). The organic layer was dried over sodium sulfate, filtered, and concentrated in vacuo. The crude product was used in next step without further purification. ESI-MS m/z=347.0 [M+Na]⁺. Calculated MW: 324.13.

### Step 3: Synthesis of tert-butyl (S)-2-(prop-2-yn-1-yl)piperazine-1-carboxylate (4)

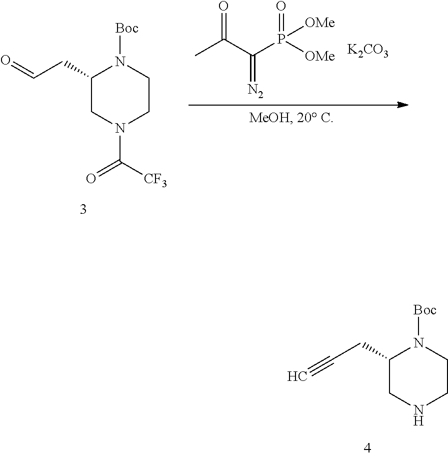

To a mixture of tert-butyl (2S)-2-(2-oxoethyl)-4-(2,2,2-trifluoroacetyl)piperazine-1-carboxylate (3) (1.82 g, 5.6 mmol, 1.0 equiv.) and K₂CO₃ (3.87 g, 28 mmol, 5.0 eq.) in anhydrous methanol (15 mL) was added dimethyl (1-diazo-2-oxopropyl)phosphonate (3.23 g, 16.8 mmol, 3.0 equiv.) dropwise at 0° C. The reaction solution was stirred at 20° C. for 16 h. The mixture was filtered and concentrated in vacuo. The residue was purified by column chromatography (eluting with petroleum ether/ethyl acetate from 0 to 100%) to afford tert-butyl (S)-2-(prop-2-yn-1-yl)piperazine-1-carboxylate (4) (0.65 g, 46% yield) as a yellow oil. ESI-MS m/z=225.2 [M+H]⁺. Calculated MW: 224.15.

### Step 4: Synthesis of 2,4-dichloro-6-(2,3-dihydro-1H-inden-1-yl)-6,7-dihydro-5H-pyrrolo[3,4-d]pyrimidine (4B)

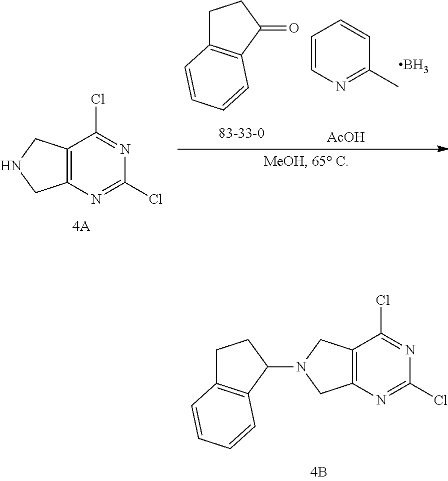

To a solution of 2,4-dichloro-6,7-dihydro-5H-pyrrolo[3,4-d]pyrimidine (4A) (5.24 g, 27.6 mmol, 1 equiv., for the procedure of 4A, please refer to the procedure of compound 1-1) and 2,3-dihydro-1H-inden-1-one (7.29 g, 55.2 mmol, 2 equiv.) in methanol (60 mL) was added 2-methylpyridine borane complex solution (8.86 g, 82.8 mmol, 3 equiv.) and acetic acid (1 mL) at 65° C. The reaction mixture was stirred at 65° C. for 2 h. The reaction mixture was concentrated in vacuo to remove methanol and to the residue was added water (80 mL), extracted with dichloromethane (100 mL×3). The organic phase was combined, and dried over sodium sulfate, filtered. The filtrate was evaporated to dryness and the residue was purified by flash column (dichloromethane/methanol=15:1) to give 2,4-dichloro-6-(2,3-dihydro-1H-inden-1-yl)-6,7-dihydro-5H-pyrrolo[3,4-d]pyrimidine (4.64 g, 54.9% yield) as white solid. 1H NMR (400 MHz, CDCl3) δ 7.34 (d, J=6.9 Hz, 5H), 7.29-7.25 (m, 2H), 7.23 (dd, J=11.3, 4.7 Hz, 1H), 4.60 (t, J=6.0 Hz, 1H), 4.13 (dd, J=26.3, 21.4 Hz, 4H), 3.14-3.00 (m, 1H), 2.97-2.82 (m, 1H), 2.29 (dt, J=13.6, 7.6 Hz, 1H), 2.16-2.04 (m, 1H).

### Step 5: Synthesis of tert-butyl (2S)-4-(2-chloro-6-(2,3-dihydro-1H-inden-1-yl)-6,7-dihydro-5H-pyrrolo[3,4-d]pyrimidin-4-yl)-2-(prop-2-yn-1-yl)piperazine-1-carboxylate (5)

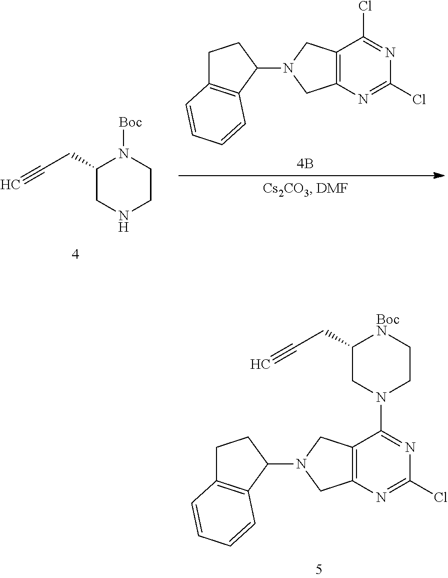

To a solution of tert-butyl (2S)-2-(prop-2-yn-1-yl)piperazine-1-carboxylate (4) (128 mg, 0.572 mmol, 1.1 equiv.) and Cs₂CO₃ (339 mg, 1.04 mmol, 2.0 equiv.) in anhydrous dimethylformamide (3 mL) was added 2,4-dichloro-6-(2,3-dihydro-1H-inden-1-yl)-5H,7H-pyrrolo[3,4-d]pyrimidine (160 mg, 0.52 mmol, 1.0 equiv.) at 0° C. The reaction solution was stirred at 0° C. for 2 h. The reaction mixture was added water (30 mL), extracted with ethyl acetate (10 mL*3). The organic layer was combined, dried over sodium sulfate, filtered, and concentrated in vacuo. The residue was purified by column chromatography (eluting with Petroleum ether/Ethyl acetate=0 to 40%) to afford tert-butyl (2S)-4-(2-chloro-6-(2,3-dihydro-1H-inden-1-yl)-6,7-dihydro-5H-pyrrolo[3,4-d]pyrimidin-4-yl)-2-(prop-2-yn-1-yl)piperazine-1-carboxylate (5) (141 mg, 49% yield) as a yellow oil. ESI-MS m/z=494.3 [M+H]⁺. Calculated MW: 493.22.

### Step 5: Synthesis of 2-chloro-6-(2,3-dihydro-1H-inden-1-yl)-4-((S)-3-(prop-2-yn-1-yl)piperazin-1-yl)-6,7-dihydro-5H-pyrrolo[3,4-d]pyrimidine (6)

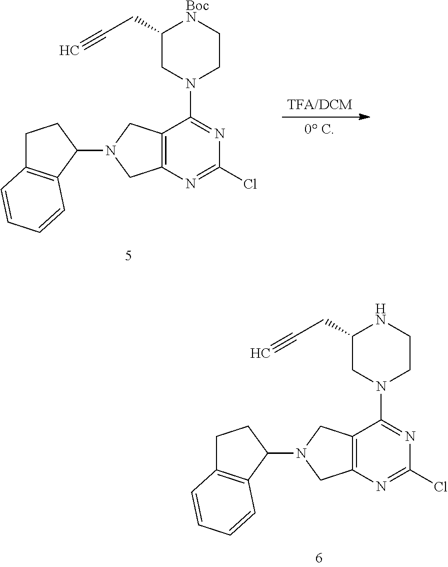 [SMILES: C#CC[C@H]1CN(c2nc(Cl)nc3c2CN(C2CCc4ccccc42)C3)CCN1C(=O)OC(C)(C)C ; "0° C." ; "TFA/DCM" ; C#CC[C@H]1CN(c2nc(Cl)nc3c2CN(C2CCc4ccccc42)C3)CCN1]

To a mixture of tert-butyl (2S)-4-[2-chloro-6-(2,3-dihydro-1H-inden-1-yl)-5H,7H-pyrrolo[3,4-d]pyrimidin-4-yl]-2-(prop-2-yn-1-yl)piperazine-1-carboxylate (5) (170 mg, 0.34 mmol) in anhydrous dichloromethane (5 mL) was added trifluoroacetic acid (1 mL) dropwise at 0° C. The reaction solution was stirred at 0° C. for 3 h. The mixture was concentrated in vacuo. pH was adjusted to 8-9 with saturated NaHCO₃ and extracted with dichloromethane (20 mL×3). The combined organic layer was dried over sodium sulfate, filtered, and concentrated in vacuo to give the crude 2-chloro-6-(2,3-dihydro-1H-inden-1-yl)-4-((S)-3-(prop-2-yn-1-yl)piperazin-1-yl)-6,7-dihydro-5H-pyrrolo[3,4-d]pyrimidine (6) (130 mg, 78% yield) directly used in next step without further purification. ESI-MS m/z=394.2 [M+H]⁺. Calculated MW: 393.17.

### Step 6: Synthesis of 6-(2,3-dihydro-1H-inden-1-yl)-2-(((S)-1-methylpyrrolidin-2-yl)methoxy)-4-((S)-3-(prop-2-yn-1-yl)piperazin-1-yl)-6,7-dihydro-5H-pyrrolo[3,4-d]pyrimidine (7)

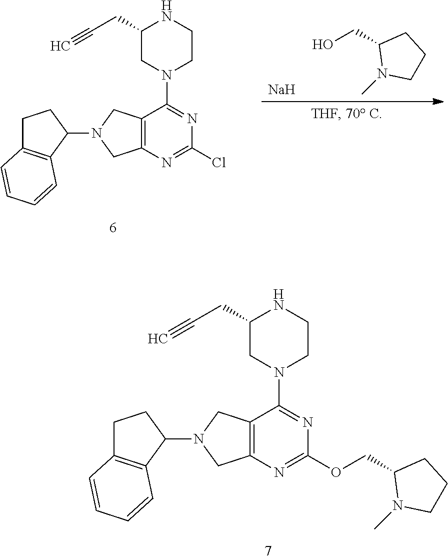

To a solution of [(2S)-1-methylpyrrolidin-2-yl] methanol (332 mg, 2.88 mmol, 8.0 eq.) in anhydrous tetrahydrofuran (2 mL) was added 60% sodium hydride (114 mg, 2.88 mmol, 8.0 equiv.) in portions. The reaction solution was stirred at 20° C. for 30 min. Then a solution of (3S)-1-[2-chloro-6-(2,3-dihydro-1H-inden-1-yl)-5H,7H-pyrrolo[3,4-d]pyrimidin-4-yl]-3-(prop-2-yn-1-yl)piperazine (6) (140 mg, 0.36 mmol, 1.0 equiv.) in tetrahydrofuran (1 mL) was added dropwise. The reaction solution was stirred at 70° C. for 0.5 h. The solution was quenched with water (10 mL) and extracted with dichloromethane (20 mL×3). The combined organic layer was dried over sodium sulfate, filtered, and concentrated in vacuo. The residue was purified by preparative-thin layer chromatography (dichloromethane/methanol=20:1) to afford 6-(2,3-dihydro-1H-inden-1-yl)-2-(((S)-1-methylpyrrolidin-2-yl)methoxy)-4-((S)-3-(prop-2-yn-1-yl)piperazin-1-yl)-6,7-dihydro-5H-pyrrolo[3,4-d]pyrimidine (7) (107 mg, 50% yield) as a yellow oil. ESI-MS m/z=473.4 [M+H]⁺. Calculated MW: 472.30

### Step 8: Synthesis of 1-((2S)-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)-2-(prop-2-yn-1-yl)piperazin-1-yl)prop-2-en-1-one (144)

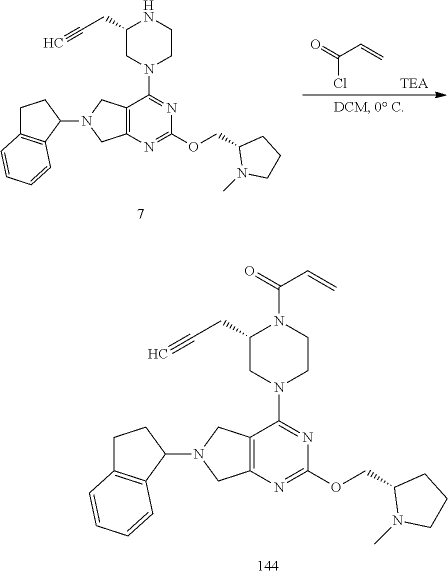

To a solution of 1-[6-(2,3-dihydro-1H-inden-1-yl)-2-{[(2S)-1-methylpyrrolidin-2-yl]methoxy}-5H,7H-pyrrolo[3,4-d]pyrimidin-4-yl]-3-(prop-2-yn-1-yl)piperazine (7) (107 mg, 0.23 mmol, 1.0 eq.) and triethylamine (47 mg, 0.46 mmol, 2.0 eq.) in anhydrous dichloromethane (3 mL) was added a solution of prop-2-enoyl chloride (25 mg, 0.28 mmol, 1.2 eq.) in dichloromethane (2 mL) at 0° C. The reaction solution was stirred at 0° C. for 10 min. The solution was quenched with water (20 mL), extracted with dichloromethane (10 mL×3). The combined organic layer was dried over sodium sulfate, filtered, and concentrated in vacuo. The residue was purified by column chromatography (eluting with acetonitrile/water (0.1% formic acid) from 5% to 60%) to afford 1-((2S)-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)-2-(prop-2-yn-1-yl)piperazin-1-yl)prop-2-en-1-one (144) (45 mg, 34% yield) as a yellow solid. ESI-MS m/z=527.3 [M+H]⁺. Calculated MW: 526.31.

### Representative Procedure K Synthesis of 1-155.

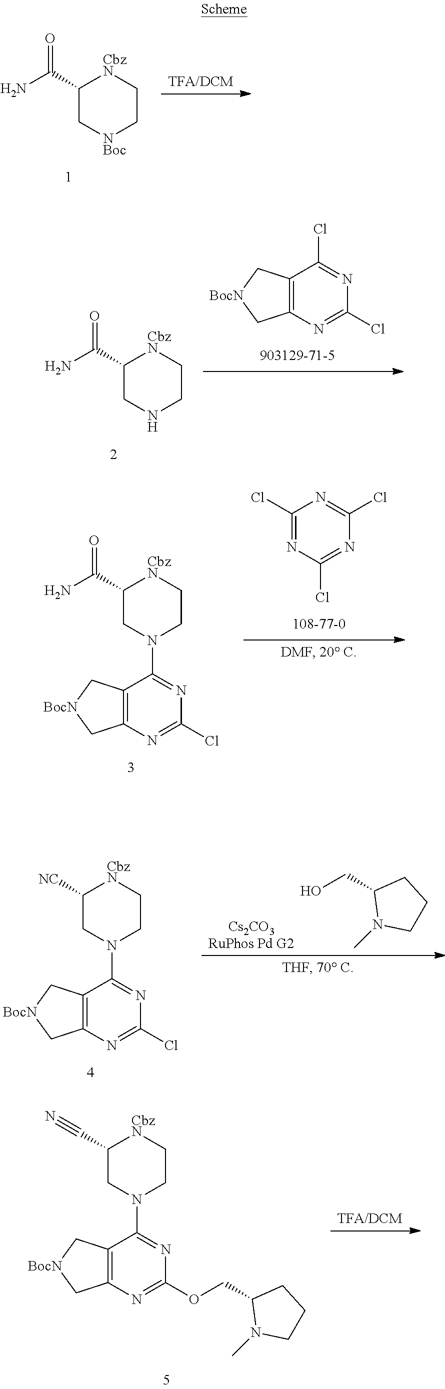

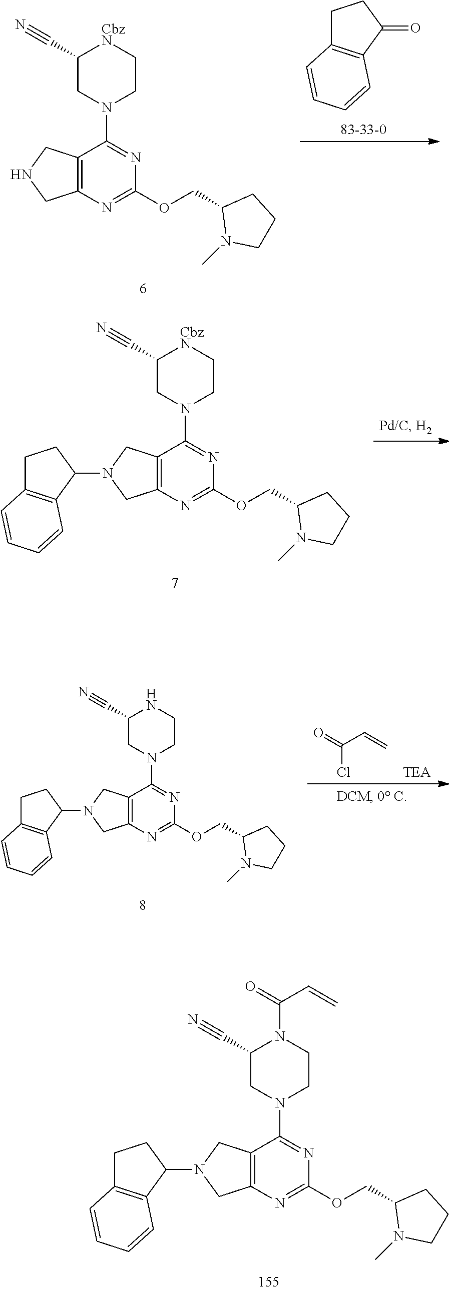

### Step 1: Synthesis of benzyl (R)-2-carbamoylpiperazine-1-carboxylate (2)

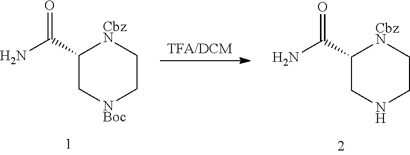 [SMILES: CC(C)(C)OC(=O)N1CCN(C(=O)OCc2ccccc2)[C@@H](C(N)=O)C1 ; "TFA/DCM" ; NC(=O)[C@H]1CNCCN1C(=O)OCc1ccccc1]

To the mixture of 1-benzyl 4-(tert-butyl) (R)-2-carbamoylpiperazine-1,4-dicarboxylate (1) (1.2 g, 3.3 mmol, 1 equiv.) in dichloromethane (5 mL) was added trifluoroacetic acid (3 mL) and the reaction was stirred at 20° C. for 2 h. The mixture was concentrated in vacuo to give crude benzyl (R)-2-carbamoylpiperazine-1-carboxylate (2) (1.2 g, ˜100% yield) as yellow oil which was used for the next step directly. ESI-MS m/z=264.13[M+H]⁺. Calculated MW: 263.13.

### Step 2: Synthesis of tert-butyl (R)-4-(4-((benzyloxy)carbonyl)-3-carbamoylpiperazin-1-yl)-2-chloro-5,7-dihydro-6H-pyrrolo[3,4-d]pyrimidine-6-carboxylate (3)

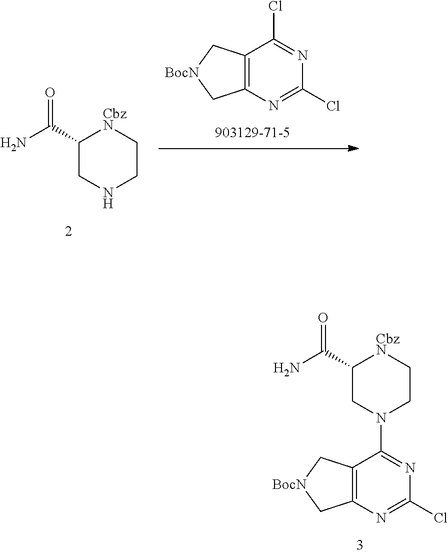 [SMILES: NC(=O)[C@H]1CNCCN1C(=O)OCc1ccccc1 ; CC(C)(C)OC(=O)N1Cc2nc(Cl)nc(Cl)c2C1 ; CC(C)(C)OC(=O)N1Cc2nc(Cl)nc(N3CCN(C(=O)OCc4ccccc4)[C@@H](C(N)=O)C3)c2C1]

The mixture of benzyl (R)-2-carbamoylpiperazine-1-carboxylate (2) (1.2 g, 3.3 mmol, 1 equiv.), diisopropylethylamine (2.1 g, 16.5 mmol, 5 equiv.) and tert-butyl 2,4-dichloro-5,7-dihydro-6H-pyrrolo[3,4-d]pyrimidine-6-carboxylate (0.9 g, 3.3 mmol, 1 equiv.) in 1,4-dioxane (15 mL) was stirred at 60° C. for 5 hours under N₂. The reaction mixture was cooled to room temperature and poured into water, and extracted with dichloromethane (20 mL×3). The organic phases were combined and washed with brine and then subsequently dried over sodium sulfate and filtered. The filtrate was evaporated to dryness and the residue was purified by silica gel chromatography (petroleum ether/ethyl acetate=5:1) to give tert-butyl (R)-4-(4-((benzyloxy)carbonyl)-3-carbamoylpiperazin-1-yl)-2-chloro-5,7-dihydro-6H-pyrrolo[3,4-d]pyrimidine-6-carboxylate (3) (1.3 g, 76.5% yield) as colorless oil. ESI-MS m/z=517.20 [M+H]⁺. Calculated MW: 516.19.

### Step 3: Synthesis of tert-butyl (R)-4-(4-((benzyloxy)carbonyl)-3-cyanopiperazin-1-yl)-2-chloro-5,7-dihydro-6H-pyrrolo[3,4-d]pyrimidine-6-carboxylate (4)

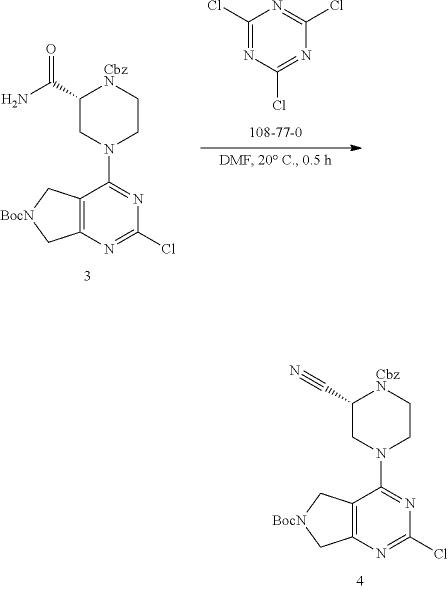 [SMILES: CC(C)(C)OC(=O)N1Cc2nc(Cl)nc(N3CCN(C(=O)OCc4ccccc4)[C@@H](C(N)=O)C3)c2C1 ; Clc1nc(Cl)nc(Cl)n1 ; "DMF, 20° C., 0.5 h" ; CC(C)(C)OC(=O)N1Cc2nc(Cl)nc(N3CCN(C(=O)OCc4ccccc4)[C@@H](C#N)C3)c2C1]

The mixture of tert-butyl (R)-4-(4-((benzyloxy)carbonyl)-3-carbamoylpiperazin-1-yl)-2-chloro-5,7-dihydro-6H-pyrrolo[3,4-d]pyrimidine-6-carboxylate (3) (1.3 g, 2.5 mmol, 1 equiv.) in dimethylformamide (10 mL) was stirred at room temperature, then 2,4,6-trichloro-1,3,5-triazine (549 mg, 3.0 mmol, 1.2 equiv.) was added and the reaction was stirred at 20° C. for 0.5 h. The reaction mixture was poured into water and extracted with dichloromethane (20 mL×3). The organic phases were combined and washed with brine. The organic layer was dried over sodium sulfate and filtered. The filtrate was evaporated to dryness and the residue was purified by chromatography silica gel (petroleum ether/ethyl acetate=1:1) to give tert-butyl (R)-4-(4-((benzyloxy)carbonyl)-3-cyanopiperazin-1-yl)-2-chloro-5,7-dihydro-6H-pyrrolo[3,4-d]pyrimidine-6-carboxylate (4) (1.2 g, 96% yield) as white solid. ESI-MS m/z=499.2 [M+H]⁺. Calculated MW: 498.18.

### Step 4: Synthesis of tert-butyl 4-((R)-4-((benzyloxy)carbonyl)-3-cyanopiperazin-1-yl)-2-(((S)-1-methylpyrrolidin-2-yl)methoxy)-5,7-dihydro-6H-pyrrolo[3,4-d]pyrimidine-6-carboxylate (5)

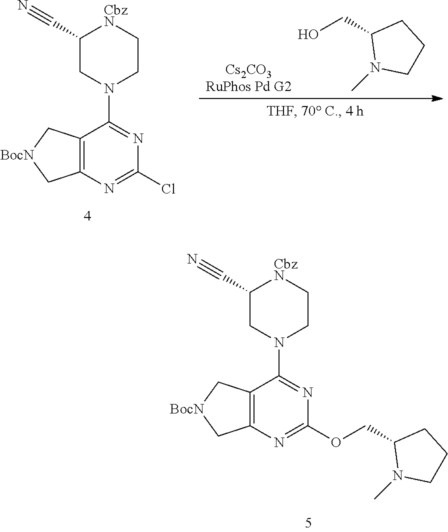

The mixture of tert-butyl (R)-4-(4-((benzyloxy)carbonyl)-3-cyanopiperazin-1-yl)-2-chloro-5,7-dihydro-6H-pyrrolo[3,4-d]pyrimidine-6-carboxylate (4) (1.2 g, 2.4 mmol, 1 equiv.), (S)-(1-methylpyrrolidin-2-yl)methanol (1.4 g, 12 mmol, 5 equiv.), Cs₂CO₃ (2.0 g, 6.0 mmol, 2.5 equiv.), and RuPhos Pd G2 (186.5 mg, 0.24 mmol, 0.1 equiv.) in tetrahydrofuran (10 mL) was stirred at 70° C. for 4 h. The reaction mixture was cooled to room temperature, and poured into water, and extracted with dichloromethane (20 mL×3). The organic phases were combined, washed with brine, dried over sodium sulfate, and filtered. The filtrate was evaporated to dryness and the residue was purified by preparative-thin layer chromatography (dichloromethane/methanol=10:1) to give tert-butyl 4-((R)-4-((benzyloxy)carbonyl)-3-cyanopiperazin-1-yl)-2-(((S)-1-methylpyrrolidin-2-yl)methoxy)-5,7-dihydro-6H-pyrrolo[3,4-d]pyrimidine-6-carboxylate (5) (340 mg, 24.6% yield) as a white solid. ESI-MS m/z=578.30 [M+H]⁺. Calculated MW: 577.30.

### Step 5: Synthesis of benzyl (R)-2-cyano-4-(2-(((S)-1-methylpyrrolidin-2-yl)methoxy)-6,7-dihydro-5H-pyrrolo[3,4-d]pyrimidin-4-yl)piperazine-1-carboxylate (6)

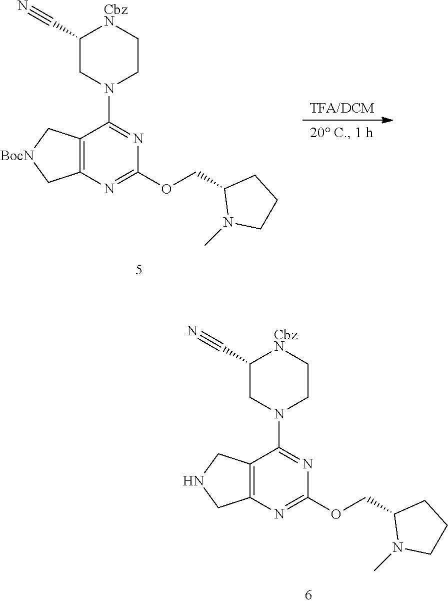 [SMILES: CN1CCC[C@H]1COc1nc2c(c(N3CCN(C(=O)OCc4ccccc4)[C@@H](C#N)C3)n1)CN(C(=O)OC(C)(C)C)C2 ; "20° C., 1 h" ; "TFA/DCM" ; CN1CCC[C@H]1COc1nc2c(c(N3CCN(C(=O)OCc4ccccc4)[C@@H](C#N)C3)n1)CNC2]

To a mixture of tert-butyl 4-((R)-4-((benzyloxy)carbonyl)-3-cyanopiperazin-1-yl)-2-(((S)-1-methylpyrrolidin-2-yl)methoxy)-5,7-dihydro-6H-pyrrolo[3,4-d]pyrimidine-6-carboxylate (5) (340 mg, 0.59 mmol, 1.0 equiv.) in dichloromethane (10 mL) was added trifluoroacetic acid (4 mL) and the reaction was stirred at 20° C. for 1 h. The reaction mixture was evaporated to dryness to give crude benzyl (R)-2-cyano-4-(2-(((S)-1-methylpyrrolidin-2-yl)methoxy)-6,7-dihydro-5H-pyrrolo[3,4-d]pyrimidin-4-yl)piperazine-1-carboxylate (6) (310 mg, 100% yield) as yellow oil. ESI-MS m/z=478.30 [M+H]⁺. Calculated MW: 477.25.

### Step 6: Synthesis of benzyl (2R)-2-cyano-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)piperazine-1-carboxylate (7)

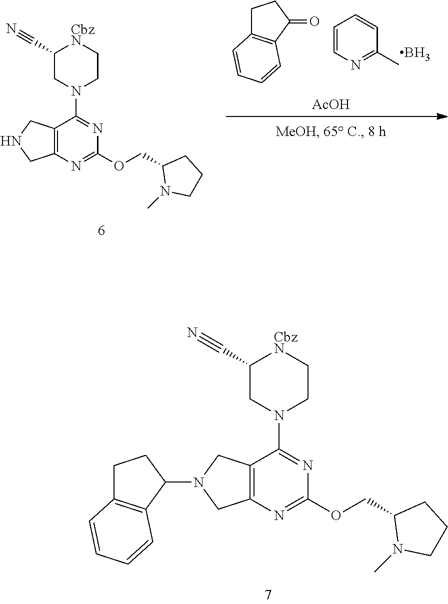

The mixture of benzyl (R)-2-cyano-4-(2-(((S)-1-methylpyrrolidin-2-yl)methoxy)-6,7-dihydro-5H-pyrrolo[3,4-d]pyrimidin-4-yl)piperazine-1-carboxylate (6) (310 mg, 0.59 mmol, 1 equiv.), 2,3-dihydro-1H-inden-1-one (235 mg, 1.78 mmol, 3 equiv.), 2-methylpyridine BH₃ (563 mg, 5.31 mmol, 9 equiv.) and acetic acid (0.2 mL) in methanol (5 mL) was stirred at 65° C. for 8 h. The reaction mixture was cooled to room temperature, poured into water, and extracted with dichloromethane (20 mL×3). The organic phases were combined, washed with brine, dried over sodium sulfate and filtered. The filtrate was evaporated to dryness and the residue was purified by preparative-thin layer chromatography (dichloromethane/methanol=10:1) to give benzyl (2R)-2-cyano-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)piperazine-1-carboxylate (7) (201 mg, 57.1% yield) as white solid. ESI-MS m/z=594.30 [M+H]⁺. Calculated MW: 593.31.

### Step 7: Synthesis of (2R)-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)piperazine-2-carbonitrile (8)

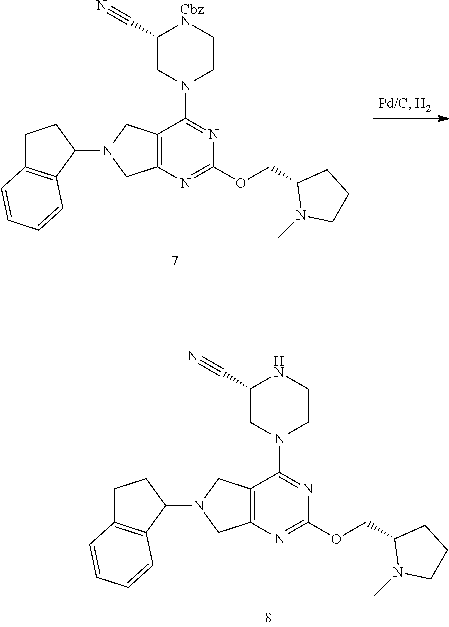 [SMILES: CN1CCC[C@H]1COc1nc2c(c(N3CCN(C(=O)OCc4ccccc4)[C@@H](C#N)C3)n1)CN(C1CCc3ccccc31)C2 ; "Pd/C, H2" ; CN1CCC[C@H]1COc1nc2c(c(N3CCN[C@@H](C#N)C3)n1)CN(C1CCc3ccccc31)C2]

To a mixture of benzyl (2R)-2-cyano-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)piperazine-1-carboxylate (7) (200 mg, 0.34 mmol, 1 equiv.) in tetrahydrofuran (5 mL) was added Pd/C (250 mg), and the reaction mixture was stirred at 20° C. for 16 h under H₂. The mixture was filtered and the filtrate was concentrated under reduced pressure to give crude product, which was purified by silica gel chromatography (dichloromethane/methanol=10:1) to give (2R)-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)piperazine-2-carbonitrile (8) (109 mg, 69.8% yield) as white solid. ESI-MS m/z=460.4 [M+H]⁺. Calculated MW: 459.27

### Step 8: Synthesis of (2R)-1-acryloyl-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)piperazine-2-carbonitrile (155)

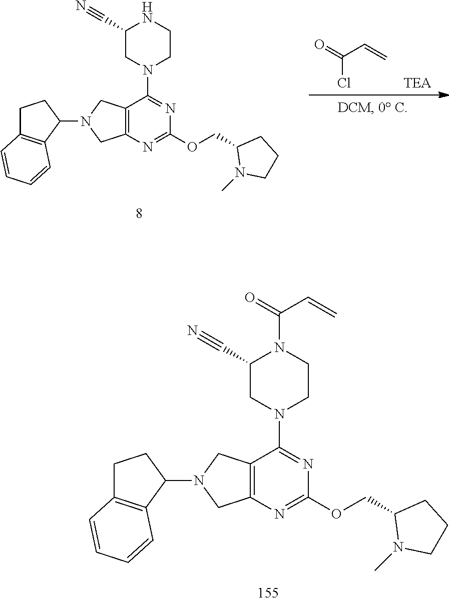

To a mixture of (2R)-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)piperazine-2-carbonitrile (8) (109 mg, 0.24 mmol, 1 equiv.) and triethylamine (120 mg, 1.18 mmol, 5 equiv.) in dichloromethane (10 mL) was added dropwise acryloyl chloride (33 mg, 0.36 mmol, 1.5 equiv.) at 0° C. After addition, the resulting mixture was stirred under nitrogen at 0° C. for 0.5 h. The reaction mixture poured into water and extracted with dichloromethane (20 mL×3). The organic phases were combined, washed with brine, dried over sodium sulfate and filtered. The filtrate was evaporated to dryness and the residue was purified by silica gel chromatography (dichloromethane/methanol=10:1) to give (2R)-1-acryloyl-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)piperazine-2-carbonitrile (155) (29 mg, 23.6% yield) as white solid. ESI-MS m/z=514.30 [M+H]⁺. Calculated MW: 513.29

### Representative Procedure L. Synthesis of 1-159.

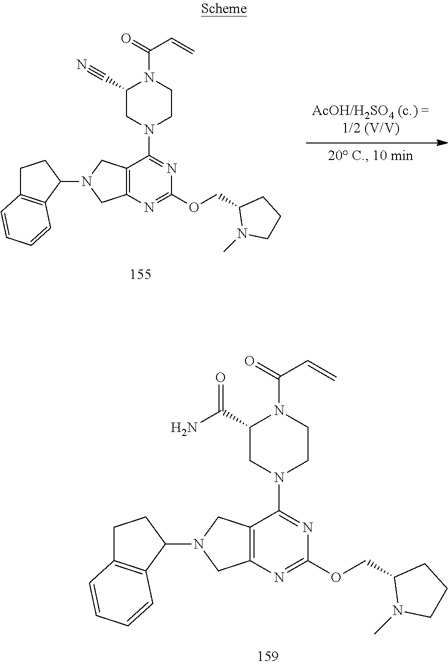

### Step 1: Synthesis of (2R)-1-acryloyl-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)piperazine-2-carboxamide (159)

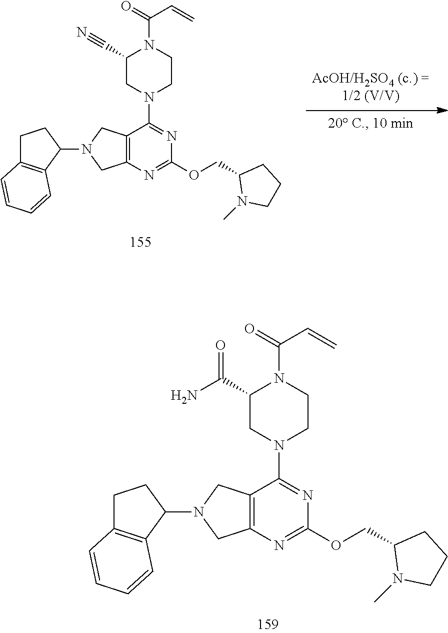

To the mixture of (2R)-1-acryloyl-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)piperazine-2-carbonitrile (155) (35 mg, 0.068 mmol, please refer to the procedure of compound 155) in acetic acid (1 mL) was added concentrated H₂SO₄ (2 mL) at 20° C. and the reaction was stirred at 20° C. for 10 min. The reaction was poured to water and adjusted to pH=9-10 by saturated aqueous Na₂CO₃ and extracted with dichloromethane (10 mL×3). The organic phases were combined, washed with brine, dried over sodium sulfate and filtered. The filtrate was evaporated to dryness and the residue was purified by preparative-thin layer chromatography (dichloromethane/methanol=10:1) to give (2R)-1-acryloyl-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)piperazine-2-carboxamide (159) (7.33 mg, 36.1% yield) as yellow solid. ESI-MS m/z=532.3[M+H]⁺. Calculated MW: 531.3

### Representative Procedure M. Synthesis of 1-162.

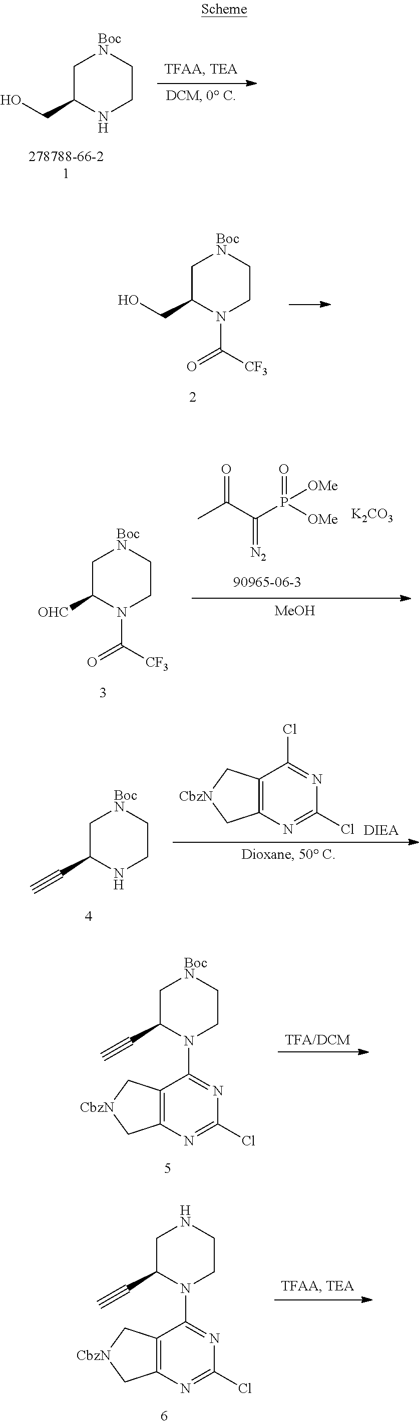

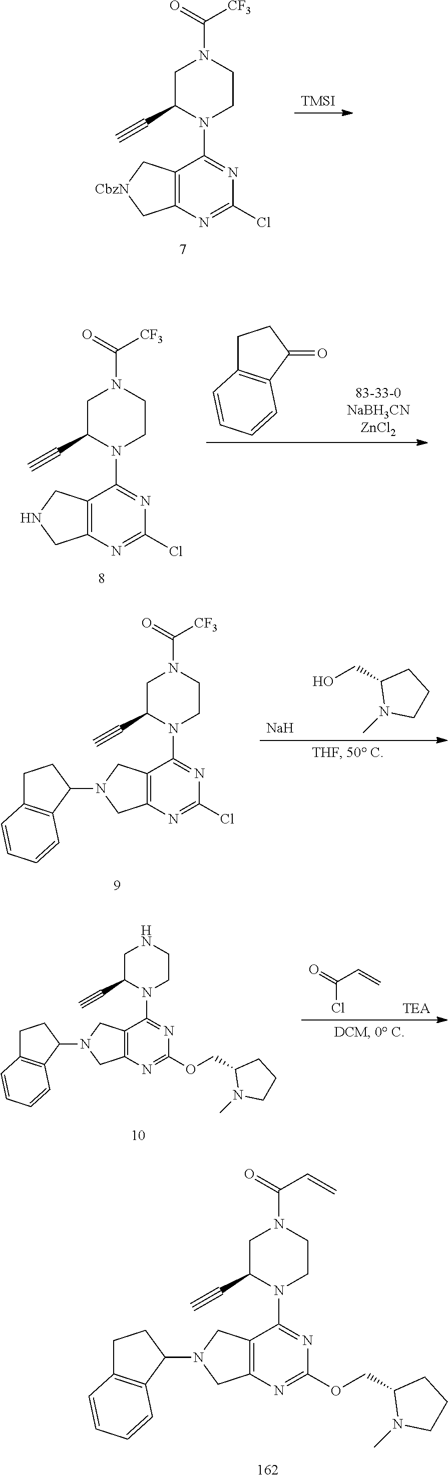

### Step 1: Synthesis of tert-butyl (3R)-3-(hydroxymethyl)-4-(2,2,2-trifluoroacetyl)piperazine-1-carboxylate (2)

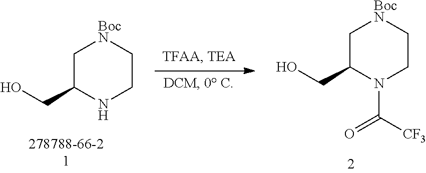 [SMILES: CC(C)(C)OC(=O)N1CCN[C@@H](CO)C1 ; "DCM, 0° C." ; "TFAA, TEA" ; CC(C)(C)OC(=O)N1CCN(C(=O)C(F)(F)F)[C@@H](CO)C1]

To a solution of tert-butyl (3R)-3-(hydroxymethyl)piperazine-1-carboxylate (1) (5.0 g, 23.12 mmol, 1 equiv.) in tetrahydrofuran (50 mL) was added trifluoroacetic anhydride (9.7 g, 46.24 mmol, 2 equiv.) and triethylamine (7.0 g, 69.36 mmol, 3 equiv.) at 0° C. The reaction mixture was stirred at 0° C. for 2 h. The reaction mixture was added water (50 mL). Then was extracted with ethyl acetate (50 mL×3). The organic phases were combined, dried over anhydrous sodium sulfate, filtered and concentrated in vacuo. The residue was purified by column chromatography (petroleum ether/ethyl acetate=1:1) to give tert-butyl (3R)-3-(hydroxymethyl)-4-(2,2,2-trifluoroacetyl)piperazine-1-carboxylate (2) (2.3 g, 31.8% yield) as a yellow oil. ¹H NMR (400 MHz, CDCl3) δ 4.33 (d, J=9.5 Hz, 1H), 4.25-3.97 (m, 3H), 3.87-3.56 (m, 3H), 2.97 (s, 3H), 1.47 (t, J=4.0 Hz, 10H).

### Step 2: Synthesis of tert-butyl (3R)-3-formyl-4-(2,2,2-trifluoroacetyl)piperazine-1-carboxylate (3)

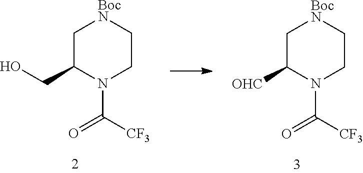 [SMILES: CC(C)(C)OC(=O)N1CCN(C(=O)C(F)(F)F)[C@@H](CO)C1 ; CC(C)(C)OC(=O)N1CCN(C(=O)C(F)(F)F)[C@@H](C=O)C1]

To a solution of tert-butyl (3R)-3-(hydroxymethyl)-4-(2,2,2-trifluoroacetyl)piperazine-1-carboxylate (2) (2.3 g, 7.4 mmol, 1 equiv.) in acetonitrile (30 mL) was added IBX (10.36 g, 37.0 mmol, 5 equiv.). The reaction mixture was stirred at 70° C. for 2 h. The solid was removed by filtration and the filtrate was concentrated in vacuo to give tert-butyl (3R)-3-formyl-4-(2,2,2-trifluoroacetyl)piperazine-1-carboxylate (3) (2.1 g, 63.51% yield, crude) as a yellow solid which was directly used for the next step. ESI-MS m/z=351.1[M+acetonitrile+1]⁺. Calculated MW: 310.11.

### Step 3: Synthesis of tert-butyl (3S)-3-ethynylpiperazine-1-carboxylate (4)

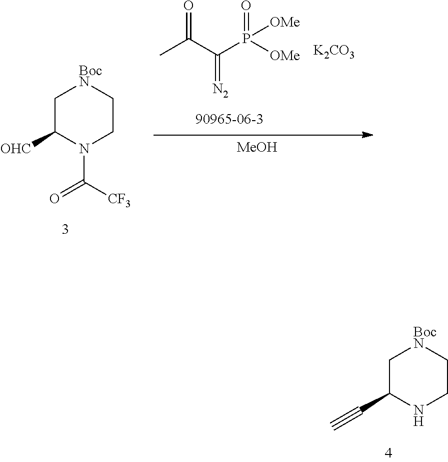

To a solution of tert-butyl (3R)-3-formyl-4-(2,2,2-trifluoroacetyl) piperazine-1-carboxylate (3) (0.85 g, 2.74 mmol, 1 equiv.) in methanol (10 mL) was added (1-diazo-2-oxo-propyl)-phosphonic acid dimethyl ester (1.58 g, 8.22 mmol, 3 equiv.) and K₂CO₃ (1.90 g, 13.7 mmol, 5 equiv.). The reaction mixture was stirred at 20° C. for 15 h. The reaction mixture was added water (40 mL) and extracted with dichloromethane (30 mL*3). The organic phases were combined, dried over anhydrous sodium sulfate, filtered and concentrated in vacuo. The residue was purified by column chromatography (petroleum ether/ethyl acetate=1:1) to give tert-butyl (3S)-3-ethynylpiperazine-1-carboxylate (4) (401 mg, 58.46% yield) as yellow oil. ¹H NMR (400 MHz, CDCl3) δ 3.75 (s, 1H), 3.60-3.45 (m, 2H), 3.30 (s, 2H), 3.04 (s, 1H), 2.71 (dd, J=14.0, 5.7 Hz, 1H), 2.27 (d, J=2.2 Hz, 1H), 1.45 (s, 9H).

### Step 4: Synthesis of tert-butyl (3S)-4-{6-[(benzyloxy)carbonyl]-2-chloro-5H,7H-pyrrolo[3,4-d]pyrimidin-4-yl}-3-ethynylpiperazine-1-carboxylate (5)

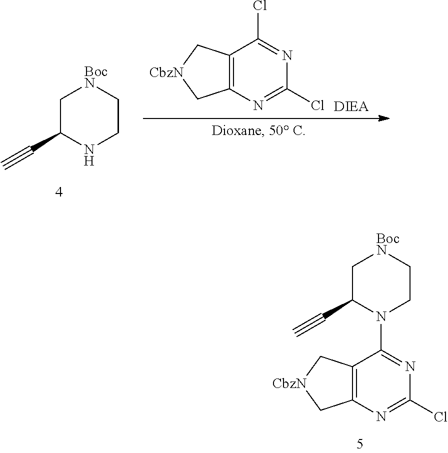

To a solution of tert-butyl (3S)-3-ethynylpiperazine-1-carboxylate (4) (300 mg, 1.43 mmol, 1 equiv.) in dioxane (3 mL) was added benzyl 2,4-dichloro-5H,7H-pyrrolo[3,4-d]pyrimidine-6-carboxylate (694 mg, 2.14 mmol, 1.5 equiv.) and diisopropylethylamine (553 mg, 2.56 mmol, 8 equiv.). The reaction mixture was stirred at 50° C. for 15 h. The mixture was concentrated in vacuo and the residue was purified by column chromatography (petroleum ether/ethyl acetate=4:1) to give tert-butyl (3S)-4-{6-[(benzyloxy)carbonyl]-2-chloro-5H,7H-pyrrolo[3,4-d]pyrimidin-4-yl}-3-ethynylpiperazine-1-carboxylate (5) (422 mg, 56.16% yield) as yellow oil. ESI-MS m/z=498.2[M+H]⁺. Calculated MW: 497.18.

### Step 5: Synthesis of benzyl 2-chloro-4-[(2S)-2-ethynylpiperazin-1-yl]-5H,7H-pyrrolo[3,4-d]pyrimidine-6-carboxylate (6)

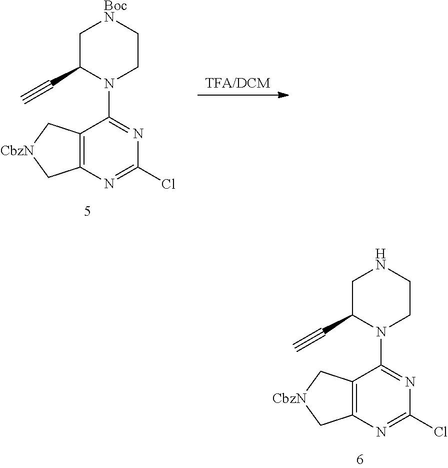 [SMILES: C#C[C@H]1CN(C(=O)OC(C)(C)C)CCN1c1nc(Cl)nc2c1CN(C(=O)OCc1ccccc1)C2 ; "TFA/DCM" ; C#C[C@H]1CNCCN1c1nc(Cl)nc2c1CN(C(=O)OCc1ccccc1)C2]

To a solution of (tert-butyl (3S)-4-{6-[(benzyloxy)carbonyl]-2-chloro-5H,7H-pyrrolo[3,4-d]pyrimidin-4-yl}-3-ethynylpiperazine-1-carboxylate (5) (420 mg, 0.84 mmol, 1 equiv.) in dichloromethane (3 mL) was added trifluoroacetic acid (1 mL). The reaction mixture was stirred at 20° C. for 2 h. The reaction mixture was concentrated in vacuo to give benzyl 2-chloro-4-[(2S)-2-ethynylpiperazin-1-yl]-5H,7H-pyrrolo[3,4-d] pyrimidine-6-carboxylate (6) (315 mg, 93.87% yield) as brown oil. ESI-MS m/z=398.1[M+H]⁺. Calculated MW: 397.13.

### Step 6: Synthesis of benzyl 2-chloro-4-[(2S)-2-ethynyl-4-(2,2,2-trifluoroacetyl)piperazin-1-yl]-5H,7H-pyrrolo[3,4-d]pyrimidine-6-carboxylate (7)

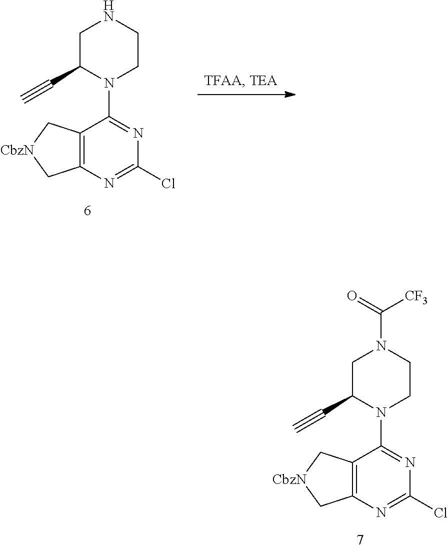 [SMILES: C#C[C@H]1CNCCN1c1nc(Cl)nc2c1CN(C(=O)OCc1ccccc1)C2 ; "TFAA, TEA" ; C#C[C@H]1CN(C(=O)C(F)(F)F)CCN1c1nc(Cl)nc2c1CN(C(=O)OCc1ccccc1)C2]

To a solution of benzyl 2-chloro-4-[(2S)-2-ethynylpiperazin-1-yl]-5H,7H-pyrrolo[3,4-d] pyrimidine-6-carboxylate (6) (420 mg 1.06 mmol, 1 equiv.) and triethylamine (215 mg 2.12 mmol, 2 equiv.) in tetrahydrofuran (10 mL) was added trifluoroacetic anhydride (445 mg 2.12 mmol, 2 equiv.). The reaction mixture was stirred at 20° C. for 2 h. The reaction mixture was added water (20 mL), extracted with dichloromethane (30 mL×3). The organic layers were combined, dried over anhydrous sodium sulfate, filtered and concentrated in vacuo. The residue was purified by column chromatography (petroleum ether/ethyl acetate=2:1) to give benzyl 2-chloro-4-[(2S)-2-ethynyl-4-(2,2,2-trifluoroacetyl)piperazin-1-yl]-5H,7H-pyrrolo[3,4-d]pyrimidine-6-carboxylate (7) (315 mg, 93.87% yield) as brown oil. ESI-MS m/z=398.1[M+H]⁺. Calculated MW: 397.13.

### Step 7: Synthesis of 1-[(3S)-4-{2-chloro-5H,6H,7H-pyrrolo[3,4-d]pyrimidin-4-yl}-3-ethynylpiperazin-1-yl]-2,2,2-trifluoroethanone (8)

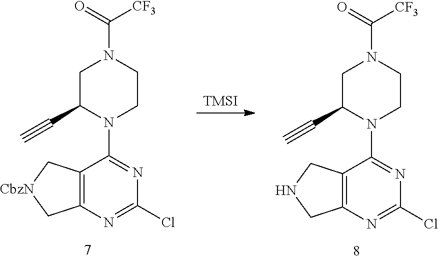 [SMILES: "TMSI" ; C#C[C@H]1CN(C(=O)C(F)(F)F)CCN1c1nc(Cl)nc2c1CN(C(=O)OCc1ccccc1)C2 ; C#C[C@H]1CN(C(=O)C(F)(F)F)CCN1c1nc(Cl)nc2c1CNC2]

To a solution of benzyl 2-chloro-4-[(2S)-2-ethynylpiperazin-1-yl]-5H,7H-pyrrolo[3,4-d] pyrimidine-6-carboxylate (7) (350 mg, 0.71 mmol, 1 equiv.) in dichloromethane (10 mL) was added trimethylsilyl iodide (795 mg, 5.67 mmol, 8 equiv.). The reaction mixture was stirred at 40° C. for 2 h. The mixture was concentrated in vacuo and the residue was purified by column chromatography (petroleum ether/ethyl acetate=1:2) to give 1-[(3S)-4-{2-chloro-5H,6H,7H-pyrrolo[3,4-d]pyrimidin-4-yl}-3-ethynylpiperazin-1-yl]-2,2,2-trifluoroethanone (8) (241 mg, 79.0% yield) as a light yellow solid. ESI-MS m/z=360.0[M+H]⁺. Calculated MW: 359.08.

### Step 8: Synthesis of 1-[(3S)-4-[2-chloro-6-(2,3-dihydro-1H-inden-1-yl)-5H,7H-pyrrolo[3,4-d]pyrimidin-4-yl]-3-ethynylpiperazin-1-yl]-2,2,2-trifluoroethanone (9)

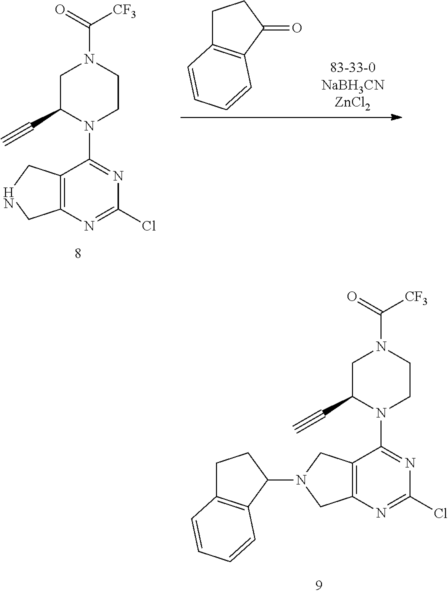 [SMILES: C#C[C@H]1CN(C(=O)C(F)(F)F)CCN1c1nc(Cl)nc2c1CNC2 ; O=C1CCc2ccccc21 ; "83-33-0NaBH3CNZnCl2" ; C#C[C@H]1CN(C(=O)C(F)(F)F)CCN1c1nc(Cl)nc2c1CN(C1CCc3ccccc31)C2]

To a solution of 1-[(3S)-4-{2-chloro-5H,6H,7H-pyrrolo[3,4-d]pyrimidin-4-yl}-3-ethynylpiperazin-1-yl]-2,2,2-trifluoroethanone (8) (200 mg, 0.56 mmol, 1 equiv.), 2,3-dihydroinden-1-one (150 mg 1.11 mmol, 2 equiv.) and ZnCl₂ (151 mg 1.11 mmol, 2 equiv.) in methanol (10 mL) was added NaBH₃CN (70 mg 1.11 mmol, 2 equiv.). The reaction mixture was stirred at 70° C. for 12 h. The reaction mixture was added water (30 mL), extracted with dichloromethane (30 mL*3). The organic layers were combined, dried over anhydrous sodium sulfate, filtered and concentrated in vacuo. The residue was purified by column chromatography (petroleum ether/ethyl acetate=2:1) to give 1-[(3S)-4-[2-chloro-6-(2,3-dihydro-1H-inden-1-yl)-5H,7H-pyrrolo[3,4-d]pyrimidin-4-yl]-3-ethynylpiperazin-1-yl]-2,2,2-trifluoroethanone (9) (181 mg, 63% yield) as a yellow oil. ESI-MS m/z=476.1[M+H]⁺. Calculated MW: 475.14.

### Step 9: Synthesis of (2S)-1-[6-(2,3-dihydro-1H-inden-1-yl)-2-{[(2S)-1-methylpyrrolidin-2-yl]methoxy}-5H,7H-pyrrolo[3,4-d]pyrimidin-4-yl]-2-ethynylpiperazine (10)

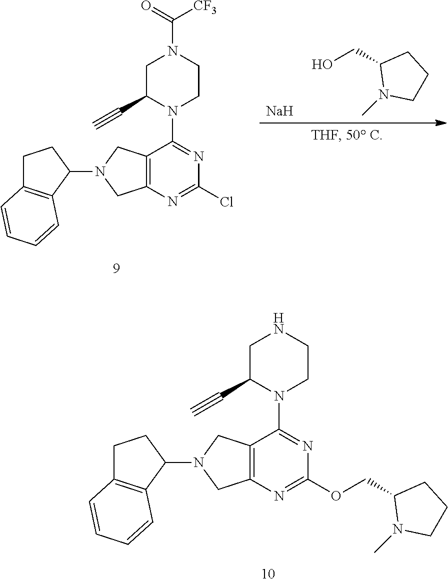

To a solution of [(2S)-1-methylpyrrolidin-2-yl] methanol (243 mg, 2.11 mmol, 5 equiv.) in tetrahydrofuran (5 mL) was added 60% sodium hydride (85 mg, 2.12 mmol, 5 equiv.). The mixture was stirred at 20° C. for 10 minutes. Then to the reaction was added 1-[(3S)-4-[2-chloro-6-(2,3-dihydro-1H-inden-1-yl)-5H,7H-pyrrolo[3,4-d]pyrimidin-4-yl]-3-ethynylpiperazin-1-yl]-2,2,2-trifluoroethanone (9) (200 mg, 0.42 mmol, 1 equiv.). The reaction mixture was stirred at 50° C. for 2 h. The reaction mixture was quenched by water (20 mL), extracted with dichloromethane (20 mL*3). The organic layers were combined, dried over anhydrous sodium sulfate, filtered and concentrated in vacuo. The residue was purified by preparative-thin layer chromatography (dichloromethane/methanol=5:1) to give (2S)-1-[6-(2,3-dihydro-1H-inden-1-yl)-2-{[(2S)-1-methylpyrrolidin-2-yl]methoxy}-5H,7H-pyrrolo[3,4-d]pyrimidin-4-yl]-2-ethynylpiperazine (10) (150 mg, 66.1% yield) as a yellow oil. ESI-MS m/z=459.3[M+H]⁺. Calculated MW: 458.28.

### Step 10: Synthesis of 1-[(3S)-4-[6-(2,3-dihydro-1H-inden-1-yl)-2-{[(2S)-1-methylpyrrolidin-2-yl]methoxy}-5H,7H-pyrrolo[3,4-d]pyrimidin-4-yl]-3-ethynylpiperazin-1-yl]prop-2-en-1-one (162)

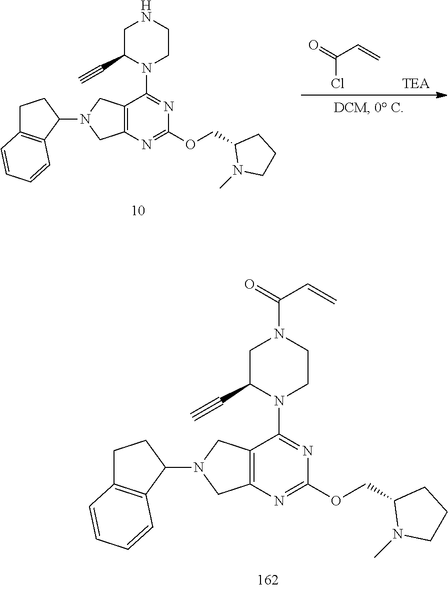

To a solution of (2S)-1-[6-(2,3-dihydro-1H-inden-1-yl)-2-{[(2S)-1-methylpyrrolidin-2-yl]methoxy}-5H,7H-pyrrolo[3,4-d]pyrimidin-4-yl]-2-ethynylpiperazine (10) (80 mg, 0.17 mmol, 1 equiv.) and triethylamine (85 mg, 2.12 mmol, 5 equiv.) in dichloromethane (5 mL) was added prop-2-enoyl chloride (200 mg 0.42 mmol, 1 equiv.) at 0° C. The reaction mixture was stirred at 0° C. for 20 min. The reaction mixture was quenched with water (10 mL) extracted with dichloromethane (10 mL*3). The organic layers were combined, dried over anhydrous sodium sulfate, filtered and concentrated in vacuo. The residue was purified by preparative-HPLC (dichloromethane/methanol=10:1) to give 1-[(3S)-4-[6-(2,3-dihydro-1H-inden-1-yl)-2-{[(2S)-1-methylpyrrolidin-2-yl]methoxy}-5H,7H-pyrrolo[3,4-d]pyrimidin-4-yl]-3-ethynylpiperazin-1-yl]prop-2-en-1-one (162) (30 mg, 66.14% yield) as a yellow oil. ESI-MS m/z=513.3[M+H]⁺. Calculated MW: 512.29.

### Representative Procedure N. Synthesis of 1-181.

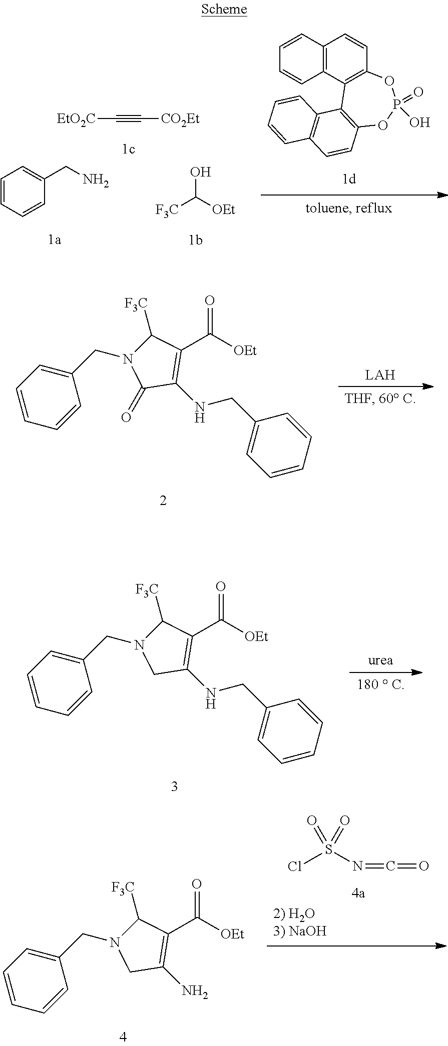

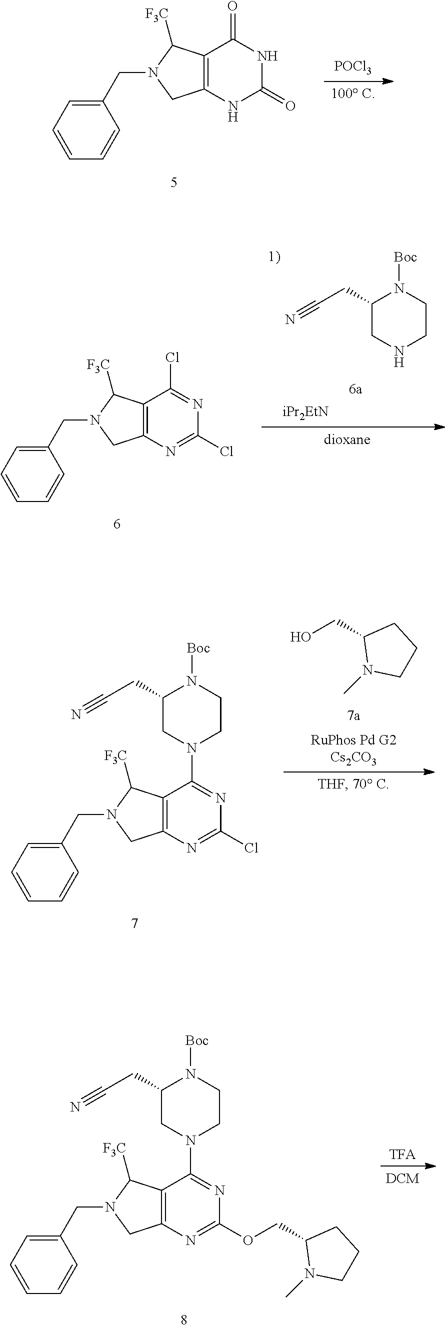

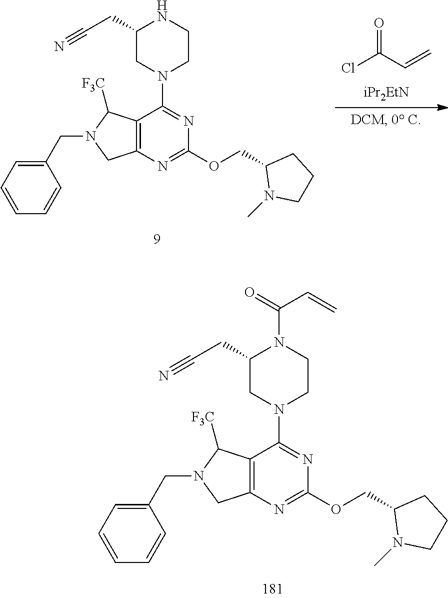

### Step 1: Synthesis of ethyl 1-benzyl-4-(benzylamino)-5-oxo-2-(trifluoromethyl)-2,5-dihydro-1H-pyrrole-3-carboxylate (2)

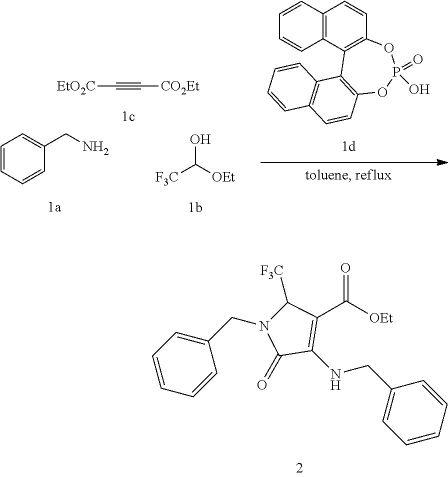 [SMILES: CCOC(=O)C#CC(=O)OCC ; O=P1(O)Oc2ccc3ccccc3c2-c2c(ccc3ccccc23)O1 ; NCc1ccccc1 ; CCOC(O)C(F)(F)F ; "toluene, reflux" ; CCOC(=O)C1=C(NCc2ccccc2)C(=O)N(Cc2ccccc2)C1C(F)(F)F]

To a toluene (100 mL) solution of phenylmethanamine (1a, 5.1 mL, 46.6 mmol) was added 1-ethoxy-2,2,2-trifluoroethan-1-ol (1b, 5.4 mL, 46.6 mmol) at room temperature. The mixture was refluxed with Dean-stark for 18 h. After cooling to room temperature, anhydrous magnesium sulfate was added to the mixture and the mixture was stirred for 30 minutes at room temperature. To the mixture was added 4-hydroxydinaphtho[2,1-d:1′,2′-f][1,3,2]dioxaphosphepine 4-oxide (1d, 1.62 g, 4.66 mmol), phenylmethanamine (1a, 5.1 mL, 46.6 mmol), and diethyl but-2-ynedioate (1c, 6.84 mL, 46.6 mmol) at room temperature. The mixture was refluxed with Dean-stark for 20 h. The mixture was filtered and the filtrate was diluted with ethyl acetate. The filtrate was washed with aqueous ammonium chloride, aqueous sodium bicarbonate, and water, dried over anhydrous magnesium sulfate, and concentrated in vacuo after filtration. Purification by silica gel column chromatography (0-40% ethyl acetate in hexane) gave crude product (12.6 g) as yellow oil. Further purification by silica gel column chromatography (10-15% ethyl acetate in hexane) gave ethyl 1-benzyl-4-(benzylamino)-5-oxo-2-(trifluoromethyl)-2,5-dihydro-1H-pyrrole-3-carboxylate (2) as yellow oil. Yield: 1.55 g, (8%). MS: 419.1 [M+H]⁺.

### Step 2: Synthesis of ethyl 1-benzyl-4-(benzylamino)-2-(trifluoromethyl)-2,5-dihydro-1H-pyrrole-3-carboxylate (3)

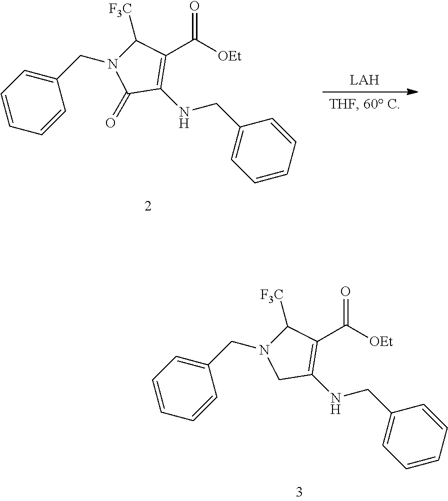 [SMILES: CCOC(=O)C1=C(NCc2ccccc2)C(=O)N(Cc2ccccc2)C1C(F)(F)F ; "LAH" ; "THF, 60° C." ; CCOC(=O)C1=C(NCc2ccccc2)CN(Cc2ccccc2)C1C(F)(F)F]

To a tetrahydrofuran (100 mL) solution of ethyl 1-benzyl-4-(benzylamino)-5-oxo-2-(trifluoromethyl)-2,5-dihydro-1H-pyrrole-3-carboxylate (2, 7.03 g, 16.8 mmol) was added lithium aluminum hydride (50.4 mL, 50.4 mmol, 1M in tetrahydrofuran) at 0° C. The mixture was stirred for 2 h at 60° C. The mixture was diluted with diethyl ether. Water (0.3 mL), 2N sodium hydroxide (0.3 mL), followed by water (0.9 mL) were added to the mixture at 0° C. The mixture was stirred for 10 minutes before magnesium sulfate was added to the mixture. The mixture was stirred for 10 minutes and filtered. The filtrate was concentrated in vacuo. Purification by silica gel column chromatography (0-30% ethyl acetate in hexane) gave ethyl 1-benzyl-4-(benzylamino)-2-(trifluoromethyl)-2,5-dihydro-1H-pyrrole-3-carboxylate (3) as yellow oil. Yield: 3.58 g (53%). MS: 405.2 [M+H]⁺.

### Step 3: Synthesis of ethyl 4-amino-1-benzyl-2-(trifluoromethyl)-2,5-dihydro-1H-pyrrole-3-carboxylate (4)

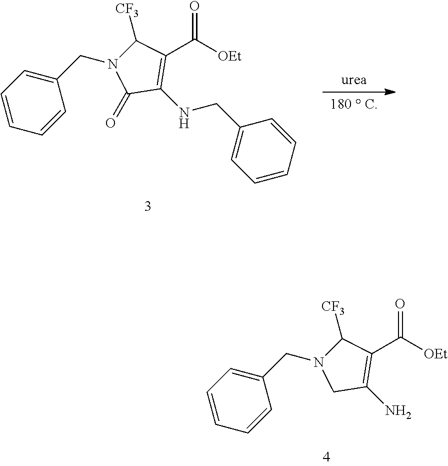 [SMILES: CCOC(=O)C1=C(NCc2ccccc2)C(=O)N(Cc2ccccc2)C1C(F)(F)F ; "180 ° C." ; "urea" ; CCOC(=O)C1=C(N)CN(Cc2ccccc2)C1C(F)(F)F]

Ethyl 1-benzyl-4-(benzylamino)-2-(trifluoromethyl)-2,5-dihydro-1H-pyrrole-3-carboxylate (3, 2.50 g, 6.18 mmol) and urea (7.43 g, 123.6 mmol) were combined and stirred for 15 h at 180° C. The mixture was suspended in water and ethyl acetate and filtered. The filtrate was extracted with ethyl acetate twice. The combined extracts were washed with brine twice, dried over anhydrous magnesium sulfate, and concentrated in vacuo after filtration to give ethyl 4-amino-1-benzyl-2-(trifluoromethyl)-2,5-dihydro-1H-pyrrole-3-carboxylate (4) as yellow oil. Yield: 1.94 g (crude). MS: 315.2 [M+H]⁺.

### Step 4: Synthesis of 6-benzyl-5-(trifluoromethyl)-1,5,6,7-tetrahydro-2H-pyrrolo[3,4-d]pyrimidine-2,4(3H)-dione (5)

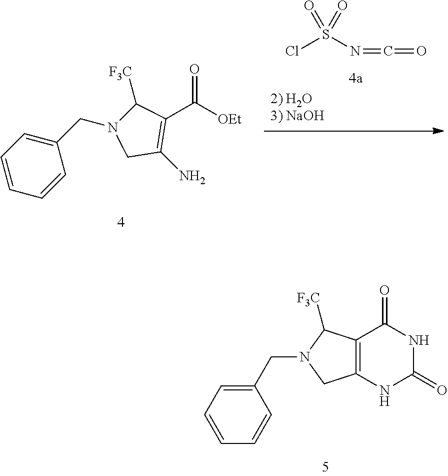

To a dichloromethane (23 mL) solution of ethyl 4-amino-1-benzyl-2-(trifluoromethyl)-2,5-dihydro-1H-pyrrole-3-carboxylate (4, 1.07 g, 3.42 mmol) was added sulfurisocyanatidic chloride (4a, 0.44 mL, 5.13 mmol) at 0° C. The mixture was stirred for 2 h at room temperature. The mixture was concentrated in vacuo. Water (70 mL) was added to the residue and the mixture was stirred for 1 h at 70° C. After cooling to room temperature, sodium hydroxide (1.37 g, 34.2 mmol) was added to the mixture at room temperature, and the mixture was stirred for 18 h at 50° C. The mixture was extracted with 20% methanol in dichloromethane three times. The combined extracts were washed with brine, dried over anhydrous magnesium sulfate, and concentrated in vacuo after filtration. Purification by silica gel column chromatography (10-70% ethyl acetate in hexane) gave 6-benzyl-5-(trifluoromethyl)-1,5,6,7-tetrahydro-2H-pyrrolo[3,4-d]pyrimidine-2,4(3H)-dione (5) as yellow oil. Yield: 130 mg, 13%. MS: 312.1 [M+H]⁺.

### Step 5: Synthesis of 6-benzyl-2,4-dichloro-5-(trifluoromethyl)-6,7-dihydro-5H-pyrrolo[3,4-d]pyrimidine (6)

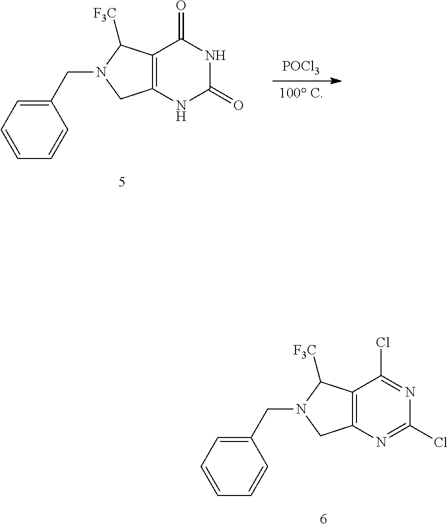 [SMILES: O=c1[nH]c2c(c(=O)[nH]1)C(C(F)(F)F)N(Cc1ccccc1)C2 ; "POCl3" ; "100° C." ; FC(F)(F)C1c2c(Cl)nc(Cl)nc2CN1Cc1ccccc1]

6-Benzyl-5-(trifluoromethyl)-1,5,6,7-tetrahydro-2H-pyrrolo[3,4-d]pyrimidine-2,4(3H)-dione (5, 130 mg, 0.42 mmol) in phosphoryl chloride (3 mL) was stirred for 6 h at 100° C. The mixture was concentrated in vacuo. The residue was diluted with ethyl acetate and washed with water, dried over anhydrous magnesium sulfate, and concentrated in vacuo after filtration. Purification by silica gel column chromatography (0-50% ethyl acetate in hexane) gave 6-benzyl-2,4-dichloro-5-(trifluoromethyl)-6,7-dihydro-5H-pyrrolo[3,4-d]pyrimidine (6) as yellow oil. Yield: 47 mg (32%). MS: 348.15 [M+H]⁺.

### Step 6: Synthesis of tert-butyl (2S)-4-(6-benzyl-2-chloro-5-(trifluoromethyl)-6,7-dihydro-5H-pyrrolo[3,4-d]pyrimidin-4-yl)-2-(cyanomethyl)piperazine-1-carboxylate (7)

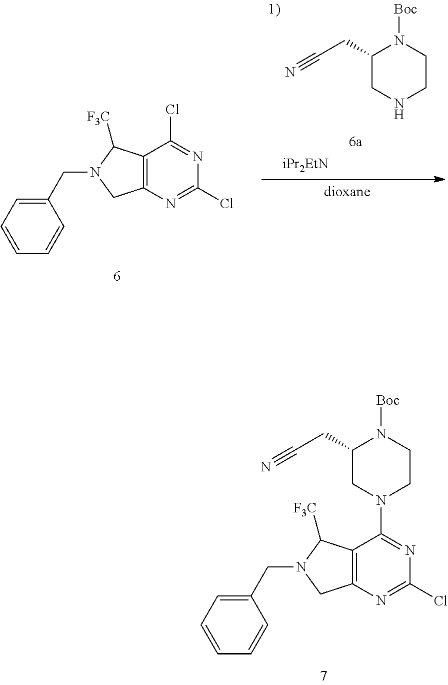 [SMILES: CC(C)(C)OC(=O)N1CCNC[C@@H]1CC#N ; FC(F)(F)C1c2c(Cl)nc(Cl)nc2CN1Cc1ccccc1 ; "dioxane" ; CC(C)(C)OC(=O)N1CCN(c2nc(Cl)nc3c2C(C(F)(F)F)N(Cc2ccccc2)C3)C[C@@H]1CC#N]

To a 1,4-dioxane (1.5 mL) solution of 6-benzyl-2,4-dichloro-5-(trifluoromethyl)-6,7-dihydro-5H-pyrrolo[3,4-d]pyrimidine (6, 47 mg, 0.13 mmol) was added tert-butyl (S)-2-(cyanomethyl)piperazine-1-carboxylate (6a, 32 mg, 0.14 mmol) and N,N-diisopropylethylamine (0.07 mL, 0.40 mmol) at room temperature. The mixture was stirred for 3 h at room temperature. The mixture was diluted with aqueous sodium bicarbonate, and extracted with ethyl acetate twice, dried over anhydrous magnesium sulfate, and concentrated in vacuo after filtration. Purification by silica gel column chromatography (0-40% ethyl acetate in hexane) gave tert-butyl (2S)-4-(6-benzyl-2-chloro-5-(trifluoromethyl)-6,7-dihydro-5H-pyrrolo[3,4-d]pyrimidin-4-yl)-2-(cyanomethyl)piperazine-1-carboxylate (7) as yellow oil. Yield: 42 mg, 58%; MS: 537.2 [M+H]⁺.

### Step 7: Synthesis of tert-butyl (2S)-4-(6-benzyl-2-(((S)-1-methylpyrrolidin-2-yl)methoxy)-5-(trifluoromethyl)-6,7-dihydro-5H-pyrrolo[3,4-d]pyrimidin-4-yl)-2-(cyanomethyl)piperazine-1-carboxylate (8)

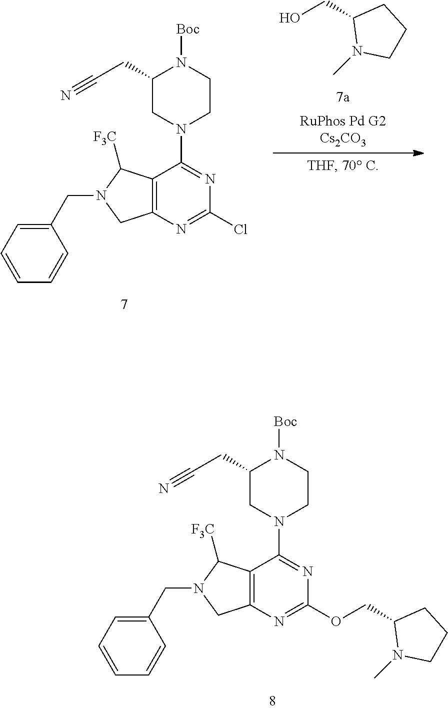

To a tetrahydrofuran (1.5 mL) solution of tert-butyl (2S)-4-(6-benzyl-2-chloro-5-(trifluoromethyl)-6,7-dihydro-5H-pyrrolo[3,4-d]pyrimidin-4-yl)-2-(cyanomethyl)piperazine-1-carboxylate (7, 42 mg, 0.080 mmol) was added (S)-(1-methylpyrrolidin-2-yl)methanol (7a, 27 mg, 0.23 mmol), cesium carbonate (127 mg, 0.39 mmol), and chloro(2-dicyclohexylphosphino-2′,6′-diisopropoxy-1,1′-biphenyl)[2-(2′-amino-1,1′-biphenyl)]palladium(II) (18 mg, 0.020 mmol) at room temperature. The mixture was bubbled with argon for 1 minutes and stirred for 3 h at 70° C. The mixture was diluted with aqueous sodium bicarbonate, and extracted with 20% methanol in dichloromethane twice, dried over anhydrous magnesium sulfate, and concentrated in vacuo after filtration. Purification by silica gel column chromatography (20-60% ethyl acetate in hexane followed by 10% methanol in dichloromethane) gave tert-butyl (2S)-4-(6-benzyl-2-(((S)-1-methylpyrrolidin-2-yl)methoxy)-5-(trifluoromethyl)-6,7-dihydro-5H-pyrrolo[3,4-d]pyrimidin-4-yl)-2-(cyanomethyl)piperazine-1-carboxylate (8) as brown oil. Yield: 35 mg (73%). MS: 616.3 [M+H]⁺.

### Step 8: Synthesis of 2-((2S)-4-(6-benzyl-2-(((S)-1-methylpyrrolidin-2-yl)methoxy)-5-(trifluoromethyl)-6,7-dihydro-5H-pyrrolo[3,4-d]pyrimidin-4-yl)piperazin-2-yl)acetonitrile (9)

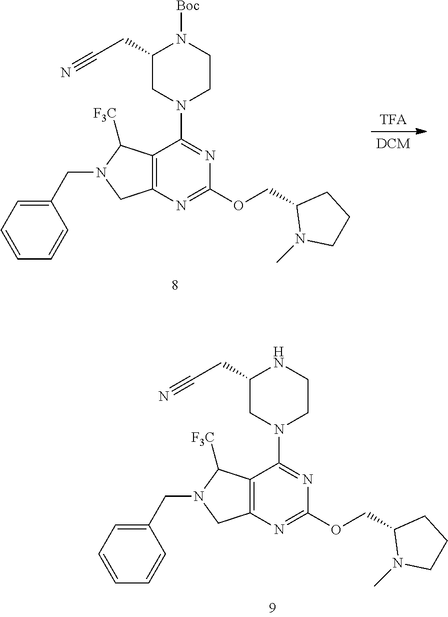

To a dichloromethane (1 mL) solution of tert-butyl (2S)-4-(6-benzyl-2-(((S)-1-methylpyrrolidin-2-yl)methoxy)-5-(trifluoromethyl)-6,7-dihydro-5H-pyrrolo[3,4-d]pyrimidin-4-yl)-2-(cyanomethyl)piperazine-1-carboxylate (8, 35 mg, 0.060 mmol) was added trifluoroacetic acid (1 mL) at 0° C. After stirring the mixture for 2 h at room temperature, the mixture was concentrated in vacuo. The residue was loaded onto a SCX column and the column was eluted with methanol, followed by 2N NH₃ in methanol. Basic fraction was concentrated in vacuo to give 2-((2S)-4-(6-benzyl-2-(((S)-1-methylpyrrolidin-2-yl)methoxy)-5-(trifluoromethyl)-6,7-dihydro-5H-pyrrolo[3,4-d]pyrimidin-4-yl)piperazin-2-yl)acetonitrile (9) as yellow oil. Yield: 19 mg (65%). MS: 516.3 [M+H]⁺.

### Step 9: Synthesis of 2-((2S)-1-acryloyl-4-(6-benzyl-2-(((S)-1-methylpyrrolidin-2-yl)methoxy)-5-(trifluoromethyl)-6,7-dihydro-5H-pyrrolo[3,4-d]pyrimidin-4-yl)piperazin-2-yl)acetonitrile (1-181)

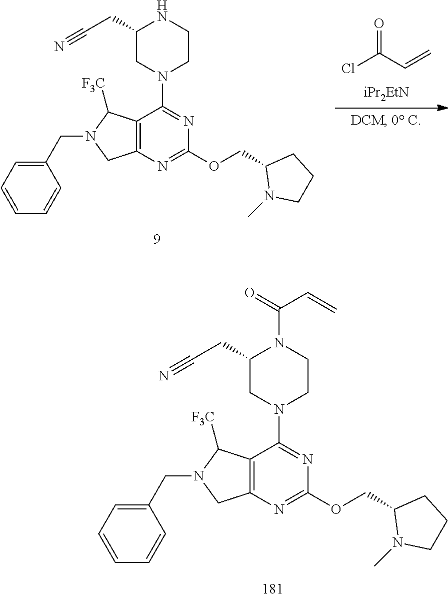

To a dichloromethane (1 mL) solution of 2-((2S)-4-(6-benzyl-2-(((S)-1-methylpyrrolidin-2-yl)methoxy)-5-(trifluoromethyl)-6,7-dihydro-5H-pyrrolo[3,4-d]pyrimidin-4-yl)piperazin-2-yl)acetonitrile (9, 19 mg, 0.040 mmol) was added N,N-diisopropylethylamine (0.11 mL, 0.11 mmol, 1M in dichloromethane) and acryloyl chloride (0.08 mL, 0.040 mmol, 0.5M in dichloromethane) at 0° C. After stirring the mixture for 1 h at 0° C., the mixture was concentrated in vacuo. Purification by preparative HPLC (C₁₈ column, 5-95% acetonitrile in water+0.1% formic acid) gave 2-((2S)-1-acryloyl-4-(6-benzyl-2-(((S)-1-methylpyrrolidin-2-yl)methoxy)-5-(trifluoromethyl)-6,7-dihydro-5H-pyrrolo[3,4-d]pyrimidin-4-yl)piperazin-2-yl)acetonitrile (1-181) as white solid as formic acid salt. Yield 3.8 mg (17%). MS 570.3 [M+H]⁺.

### Representative Procedure O. Synthesis of 1-197.

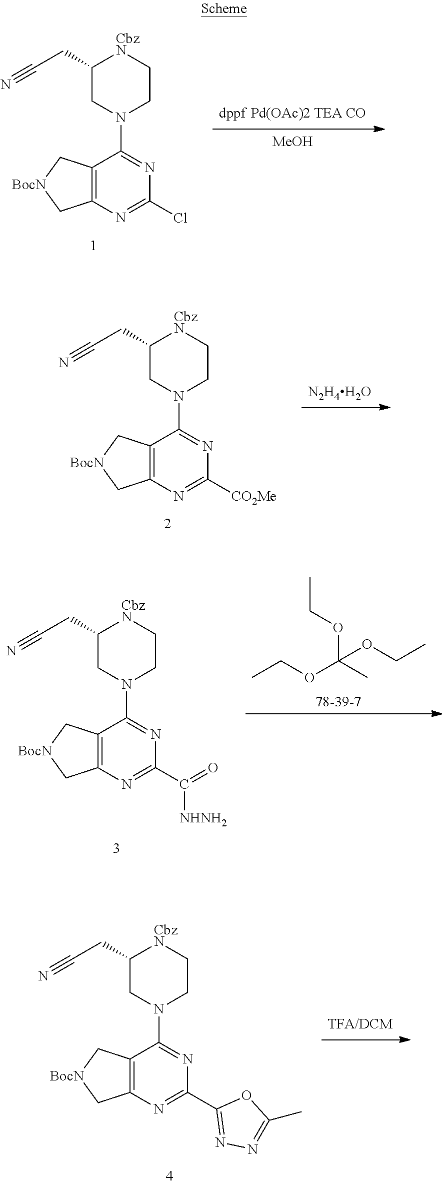

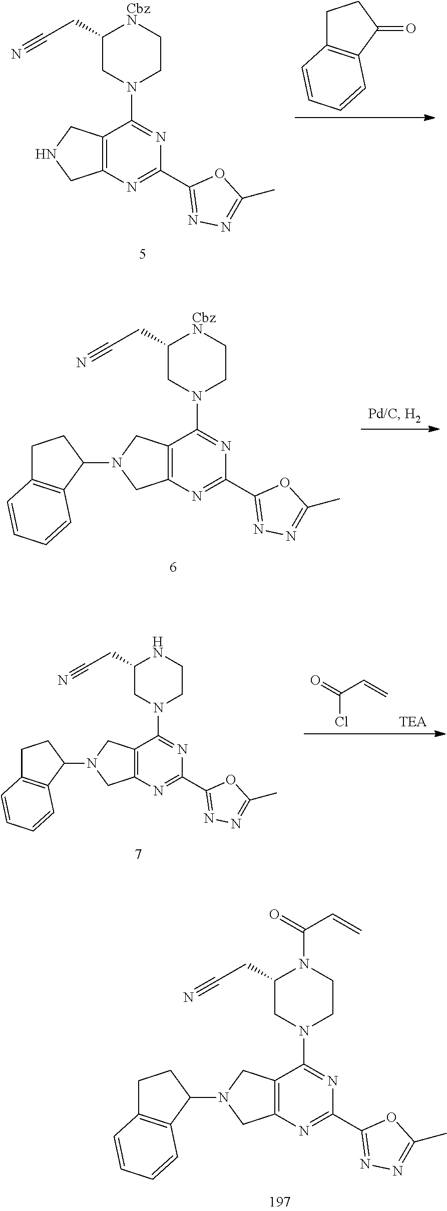

### Step 1: Synthesis of 6-(tert-butyl) 2-methyl (S)-4-(4-((benzyloxy)carbonyl)-3-(cyanomethyl)piperazin-1-yl)-5,7-dihydro-6H-pyrrolo[3,4-d]pyrimidine-2,6-dicarboxylate (2)

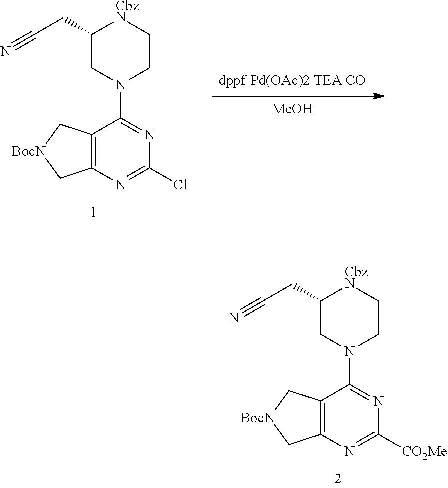 [SMILES: "MeOH" ; CC(C)(C)OC(=O)N1Cc2nc(Cl)nc(N3CCN(C(=O)OCc4ccccc4)[C@@H](CC#N)C3)c2C1 ; COC(=O)c1nc2c(c(N3CCN(C(=O)OCc4ccccc4)[C@@H](CC#N)C3)n1)CN(C(=O)OC(C)(C)C)C2]

The mixture of tert-butyl (S)-4-(4-((benzyloxy)carbonyl)-3-(cyanomethyl)piperazin-1-yl)-2-chloro-5,7-dihydro-6H-pyrrolo[3,4-d]pyrimidine-6-carboxylate (1) (1.2 g, 2.3 mmol, 1 equiv., for the procedure of 1, please refer to the procedure of compound 1-7), dppf (127 mg, 0.23 mmol, 0.1 equiv.), Pd(OAc)₂ (51.5 mg, 0.23 mmol, 0.1 equiv.) and triethylamine (581 mg, 5.75 mmol, 2.5 equiv.) in methanol (15 mL) was stirred at 70° C. under carbon monoxide for 12 h. The reaction mixture was cooled to room temperature and poured into water and extracted with dichloromethane (20 mL×3). The organic phases were combined, washed with brine, dried over sodium sulfate and filtered. The filtrate was evaporated to dryness and the residue was purified by chromatography silica gel (petroleum ether/ethyl acetate=5:1) to give 6-(tert-butyl) 2-methyl (S)-4-(4-((benzyloxy)carbonyl)-3-(cyanomethyl)piperazin-1-yl)-5,7-dihydro-6H-pyrrolo[3,4-d]pyrimidine-2,6-dicarboxylate (2) (1.2 g, 97.6% yield) as colorless oil. ESI-MS m/z=537.3[M+H]⁺. Calculated MW: 536.5.

### Step 2: Synthesis of tert-butyl (S)-4-(4-((benzyloxy)carbonyl)-3-(cyanomethyl)piperazin-1-yl)-2-(hydrazinecarbonyl)-5,7-dihydro-6H-pyrrolo[3,4-d]pyrimidine-6-carboxylate (3)

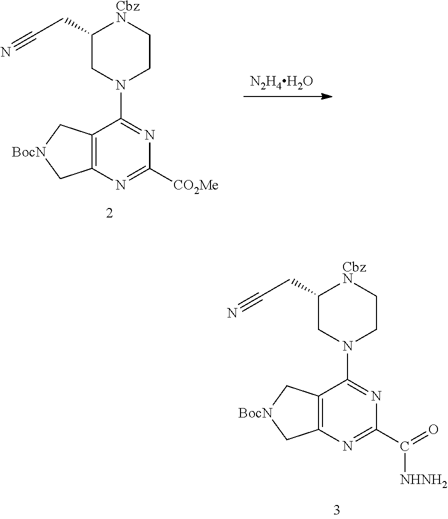 [SMILES: COC(=O)c1nc2c(c(N3CCN(C(=O)OCc4ccccc4)[C@@H](CC#N)C3)n1)CN(C(=O)OC(C)(C)C)C2 ; "N2H4·H2O" ; CC(C)(C)OC(=O)N1Cc2nc(C(=O)NN)nc(N3CCN(C(=O)OCc4ccccc4)[C@@H](CC#N)C3)c2C1]

To the mixture of 6-(tert-butyl) 2-methyl (S)-4-(4-((benzyloxy)carbonyl)-3-(cyanomethyl)piperazin-1-yl)-5,7-dihydro-6H-pyrrolo[3,4-d]pyrimidine-2,6-dicarboxylate (2) (1.2 g, 2.2 mmol, 1 equiv.) in methanol (15 mL) was added hydrazine (770 mg, 22 mmol, 10 equiv.), and the mixture was stirred at 60° C. for 2 h. The reaction mixture was cooled to room temperature and poured into water and extracted with dichloromethane (20 mL×3). The organic phases were combined, washed with brine, dried over sodium sulfate and filtered. The filtrate was evaporated to dryness and the residue was purified by silica gel chromatography (methanol/dichloromethane=1:50) to give tert-butyl (S)-4-(4-((benzyloxy)carbonyl)-3-(cyanomethyl)piperazin-1-yl)-2-(hydrazinecarbonyl)-5,7-dihydro-6H-pyrrolo[3,4-d]pyrimidine-6-carboxylate (3) (1.05 g, 87.5% yield) as white solid. ESI-MS m/z=537.3 [M+H]⁺. Calculated MW: 536.2.

### Step 3: Synthesis of tert-butyl (S)-4-(4-((benzyloxy)carbonyl)-3-(cyanomethyl)piperazin-1-yl)-2-(5-methyl-1,3,4-oxadiazol-2-yl)-5,7-dihydro-6H-pyrrolo[3,4-d]pyrimidine-6-carboxylate (4)

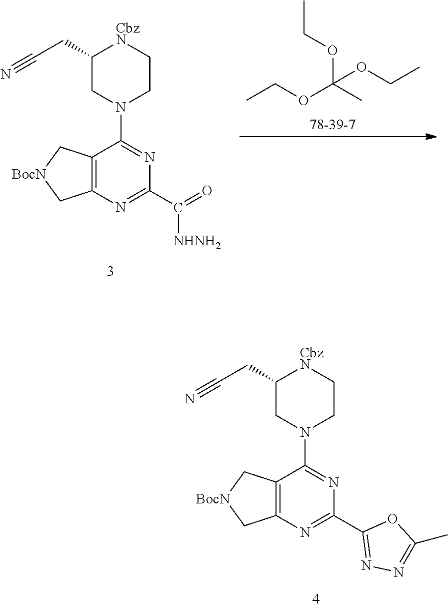

To a mixture of tert-butyl (S)-4-(4-((benzyloxy)carbonyl)-3-(cyanomethyl)piperazin-1-yl)-2-(hydrazinecarbonyl)-5,7-dihydro-6H-pyrrolo[3,4-d]pyrimidine-6-carboxylate (3) (1.05 g, 1.96 mmol, 1 equiv.) and NH₄Cl (312 mg, 5.88 mmol, 3 equiv.) in N-methyl pyrrolidinone (3 mL) was added 1,1,1-triethoxyethane (953 mg, 5.88 mmol, 3 equiv.) and the reaction mixture was stirred at 130° C. for 5 h. The reaction mixture was cooled to room temperature and poured into water and extracted with dichloromethane (20 mL×3). The organic phase was combined, washed with brine. The organic layer was dried over sodium sulfate and filtered. The filtrate was evaporated to dryness and the residue was purified by silica gel chromatography (dichloromethane/methanol=50:1) to give tert-butyl (S)-4-(4-((benzyloxy)carbonyl)-3-(cyanomethyl)piperazin-1-yl)-2-(5-methyl-1,3,4-oxadiazol-2-yl)-5,7-dihydro-6H-pyrrolo[3,4-d]pyrimidine-6-carboxylate (4) (340 mg, 31% yield) as white solid. ESI-MS m/z=561.3 [M+H]⁺. Calculated MW: 560.2.

### Step 4: Synthesis of benzyl (S)-2-(cyanomethyl)-4-(2-(5-methyl-1,3,4-oxadiazol-2-yl)-6,7-dihydro-5H-pyrrolo[3,4-d]pyrimidin-4-yl)piperazine-1-carboxylate (5)

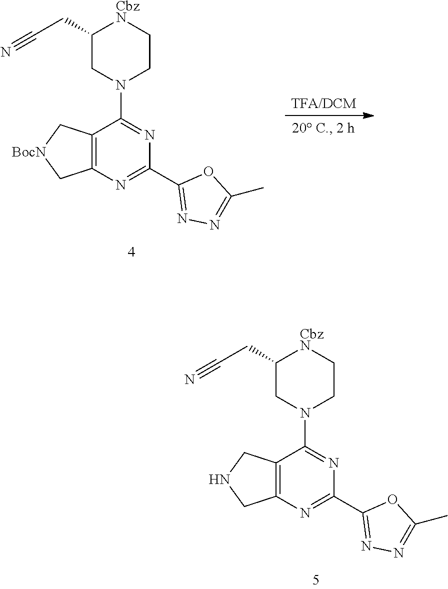 [SMILES: Cc1nnc(-c2nc3c(c(N4CCN(C(=O)OCc5ccccc5)[C@@H](CC#N)C4)n2)CN(C(=O)OC(C)(C)C)C3)o1 ; "20° C., 2 h" ; "TFA/DCM" ; Cc1nnc(-c2nc3c(c(N4CCN(C(=O)OCc5ccccc5)[C@@H](CC#N)C4)n2)CNC3)o1]

To the mixture of tert-butyl (S)-4-(4-((benzyloxy)carbonyl)-3-(cyanomethyl)piperazin-1-yl)-2-(5-methyl-1,3,4-oxadiazol-2-yl)-5,7-dihydro-6H-pyrrolo[3,4-d]pyrimidine-6-carboxylate (4) (340 mg, 0.61 mmol, 1 equiv.) in dichloromethane (5 mL) was added trifluoroacetic acid (3 mL) slowly and the reaction mixture was stirred at 20° C. for 2 h. The reaction mixture was evaporated to give crude benzyl (S)-2-(cyanomethyl)-4-(2-(5-methyl-1,3,4-oxadiazol-2-yl)-6,7-dihydro-5H-pyrrolo[3,4-d]pyrimidin-4-yl)piperazine-1-carboxylate (5) (310 mg, 100% yield) as a yellow oil. ESI-MS m/z=461.2[M+H]⁺. Calculated MW: 460.20.

### Step 5: Synthesis of benzyl (2S)-2-(cyanomethyl)-4-(6-(2,3-dihydro-1H-inden-1-yl)-2-(5-methyl-1,3,4-oxadiazol-2-yl)-6,7-dihydro-5H-pyrrolo[3,4-d]pyrimidin-4-yl)piperazine-1-carboxylate (6)

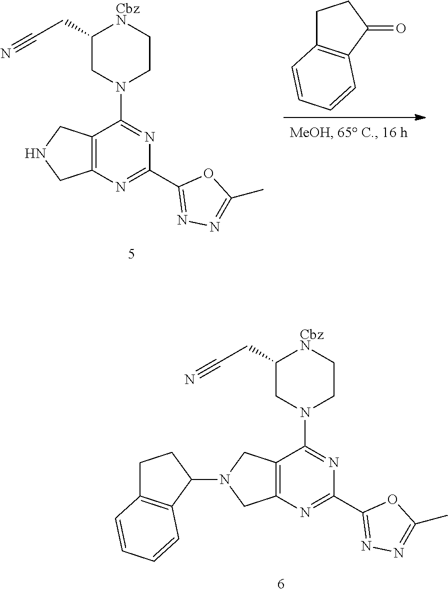 [SMILES: Cc1nnc(-c2nc3c(c(N4CCN(C(=O)OCc5ccccc5)[C@@H](CC#N)C4)n2)CNC3)o1 ; O=C1CCc2ccccc21 ; Cc1nnc(-c2nc3c(c(N4CCN(C(=O)OCc5ccccc5)[C@@H](CC#N)C4)n2)CN(C2CCc4ccccc42)C3)o1]

The mixture of benzyl (S)-2-(cyanomethyl)-4-(2-(5-methyl-1,3,4-oxadiazol-2-yl)-6,7-dihydro-5H-pyrrolo[3,4-d]pyrimidin-4-yl)piperazine-1-carboxylate (5) (310 mg, 0.67 mmol, 1 equiv.), 2,3-dihydro-1H-inden-1-one (265.3 mg, 2.01 mmol, 3 equiv.), 2-methylpyridine-BH₃ complex (562.9 mg, 5.31 mmol, 7.92 equiv.) and acetic acid (0.2 mL) in methanol (5 mL) was stirred at 65° C. for 16 h. The reaction mixture was cooled to 20° C., poured into water, and extracted with dichloromethane (20 mL×3). The organic phases were combined, washed with brine, dried over sodium sulfate and filtered. The filtrate was evaporated to dryness and the residue was purified by preparative-thin layer chromatography (dichloromethane/methanol=10:1) to give benzyl (2S)-2-(cyanomethyl)-4-(6-(2,3-dihydro-1H-inden-1-yl)-2-(5-methyl-1,3,4-oxadiazol-2-yl)-6,7-dihydro-5H-pyrrolo[3,4-d]pyrimidin-4-yl)piperazine-1-carboxylate (6) (189 mg, 48.9% yield) as a white solid. ESI-MS m/z=577.3 [M+H]⁺. Calculated MW: 576.26.

### Step 6: Synthesis of 2-((2S)-4-(6-(2,3-dihydro-1H-inden-1-yl)-2-(5-methyl-1,3,4-oxadiazol-2-yl)-6,7-dihydro-5H-pyrrolo[3,4-d]pyrimidin-4-yl)piperazin-2-yl)acetonitrile (7)

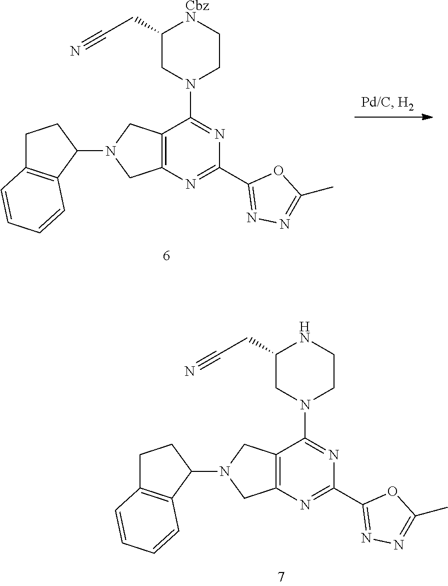 [SMILES: Cc1nnc(-c2nc3c(c(N4CCN(C(=O)OCc5ccccc5)[C@@H](CC#N)C4)n2)CN(C2CCc4ccccc42)C3)o1 ; "Pd/C, H2" ; Cc1nnc(-c2nc3c(c(N4CCN[C@@H](CC#N)C4)n2)CN(C2CCc4ccccc42)C3)o1]

To a mixture of benzyl (2S)-2-(cyanomethyl)-4-(6-(2,3-dihydro-1H-inden-1-yl)-2-(5-methyl-1,3,4-oxadiazol-2-yl)-6,7-dihydro-5H-pyrrolo[3,4-d]pyrimidin-4-yl)piperazine-1-carboxylate (6) (189 mg, 0.33 mmol, 1 equiv.) in tetrahydrofuran (5 mL) was added 10% Pd/C (250 mg) and the reaction mixture was stirred at 20° C. under H₂ for 16 h. The mixture was filtered and concentrated under reduced pressure to give a crude product, which was purified by silica gel chromatography (dichloromethane/methanol=10:1) to give 2-((2S)-4-(6-(2,3-dihydro-1H-inden-1-yl)-2-(5-methyl-1,3,4-oxadiazol-2-yl)-6,7-dihydro-5H-pyrrolo[3,4-d]pyrimidin-4-yl)piperazin-2-yl)acetonitrile (7) (69 mg, 47.3% yield) as a white solid. ESI-MS m/z=443.3 [M+H]⁺. Calculated MW: 442.22.

### Step 7: Synthesis of 2-((2S)-1-acryloyl-4-(6-(2,3-dihydro-1H-inden-1-yl)-2-(5-methyl-1,3,4-oxadiazol-2-yl)-6,7-dihydro-5H-pyrrolo[3,4-d]pyrimidin-4-yl)piperazin-2-yl)acetonitrile (197)

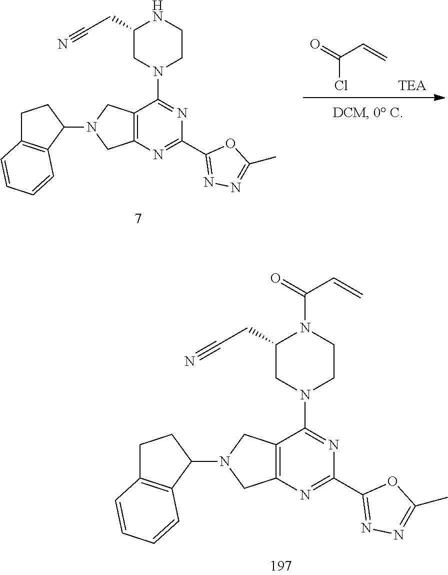

To a mixture of 2-((2S)-4-(6-(2,3-dihydro-1H-inden-1-yl)-2-(5-methyl-1,3,4-oxadiazol-2-yl)-6,7-dihydro-5H-pyrrolo[3,4-d]pyrimidin-4-yl)piperazin-2-yl)acetonitrile (7) (69 mg, 0.16 mmol, 1 equiv.) and triethylamine (40 mg, 0.4 mmol, 2.5 equiv.) in dichloromethane (5 mL) was added dropwise acryloyl chloride (22 mg, 0.24 mmol, 1.5 equiv.) at 0° C. After addition, the resulting mixture was stirred under nitrogen at 0° C. for 0.5 h. The reaction mixture poured into water and extracted with dichloromethane (20 mL×3). The organic phases were combined, washed with brine. The organic layer was dried over sodium sulfate and filtered. The filtrate was evaporated to dryness and the residue was purified by silica gel chromatography (dichloromethane/methanol=10:1) to give 2-((2S)-1-acryloyl-4-(6-(2,3-dihydro-1H-inden-1-yl)-2-(5-methyl-1,3,4-oxadiazol-2-yl)-6,7-dihydro-5H-pyrrolo[3,4-d]pyrimidin-4-yl)piperazin-2-yl)acetonitrile (197) (9.31 mg, 11.7% yield) as a white solid. ESI-MS m/z=497.3[M+H]⁺. Calculated MW: 496.23.

### Representative Procedure P. Synthesis of 1-198.

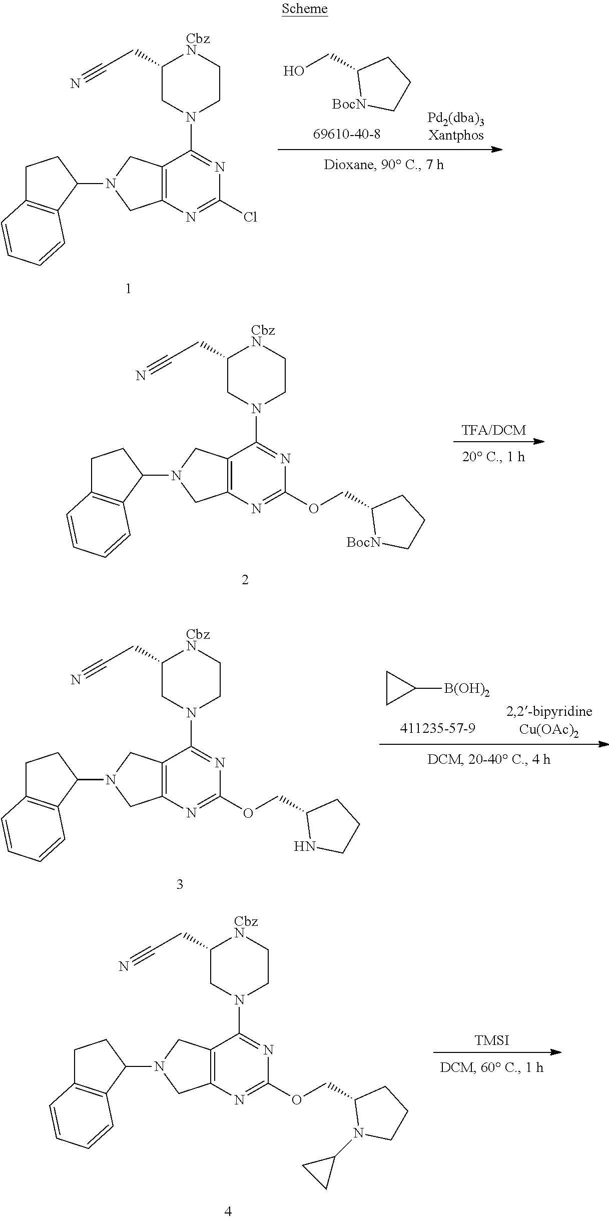

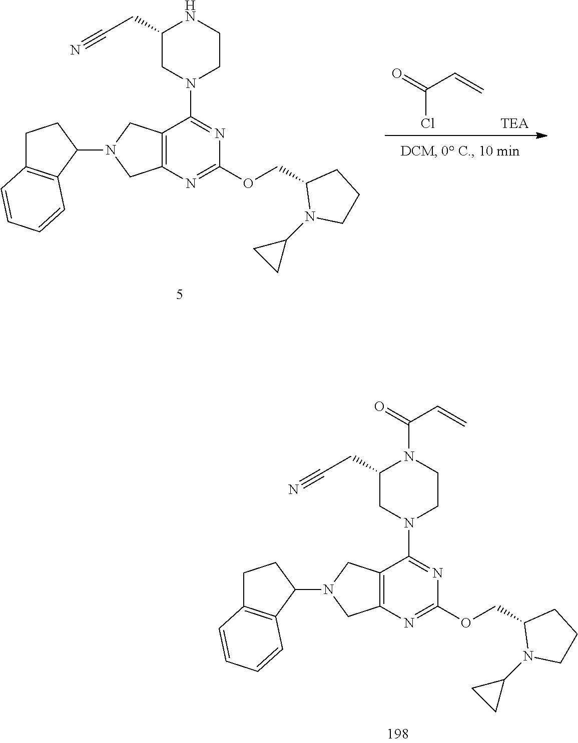

### Step 1: Synthesis of benzyl (2S)-4-(2-(((S)-1-(tert-butoxycarbonyl)pyrrolidin-2-yl)methoxy)-6-(2,3-dihydro-1H-inden-1-yl)-6,7-dihydro-5H-pyrrolo[3,4-d]pyrimidin-4-yl)-2-(cyanomethyl)piperazine-1-carboxylate (2)

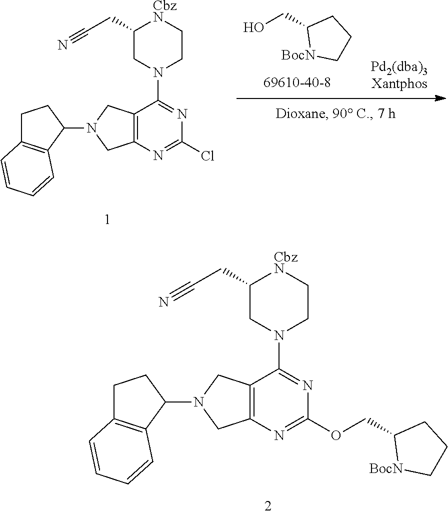 [SMILES: N#CC[C@H]1CN(c2nc(Cl)nc3c2CN(C2CCc4ccccc42)C3)CCN1C(=O)OCc1ccccc1 ; CC(C)(C)OC(=O)N1CCC[C@H]1CO ; "Pd2(dba)3Xantphos" ; CC(C)(C)OC(=O)N1CCC[C@H]1COc1nc2c(c(N3CCN(C(=O)OCc4ccccc4)[C@@H](CC#N)C3)n1)CN(C1CCc3ccccc31)C2]

To a mixture of benzyl (2S)-4-[2-chloro-6-(2,3-dihydro-1H-inden-1-yl)-5H,7H-pyrrolo[3,4-d]pyrimidin-4-yl]-2-(cyanomethyl)piperazine-1-carboxylate (1) (0.75 g, 1.26 mmol, 1 equiv., for the procedure of 1, please refer to the procedure of compound 129) and tert-butyl (2S)-2-(hydroxymethyl)pyrrolidine-1-carboxylate (3.80 g, 19.00 mmol, 5 equiv.) in dioxane (50 mL) was added Xantphos (0.66 g, 1.14 mmol, 0.3 equiv.) and Cs₂CO₃ (3.71 g, 11.40 mmol, 3 equiv.) under N₂ at 20° C. Then to the mixture was added Pd₂(dba)₃ (1.04 g, 1.14 mmol, 0.3 equiv.) at 20° C. under N₂. The resulting mixture was stirred at 90° C. for 7 h. The reaction mixture was quenched by water (50 mL) and extracted by ethyl acetate (50 mL*3). The organic phases were combined, dried over sodium sulfate, filtered and concentrated in vacuo. The residue was purified by silica gel chromatography (dichloromethane:methanol=10:1) to give benzyl (2S)-4-(2-(((S)-1-(tert-butoxycarbonyl)pyrrolidin-2-yl)methoxy)-6-(2,3-dihydro-1H-inden-1-yl)-6,7-dihydro-5H-pyrrolo[3,4-d]pyrimidin-4-yl)-2-(cyanomethyl)piperazine-1-carboxylate (2) (1.5 g, 56.9% yield) as yellow solid. ESI-MS m/z=694.3 [M+H]⁺. Calculated MW: 693.36.

### Step 2: Synthesis of benzyl (2S)-2-(cyanomethyl)-4-(6-(2,3-dihydro-1H-inden-1-yl)-2-(((S)-pyrrolidin-2-yl)methoxy)-6,7-dihydro-5H-pyrrolo[3,4-d]pyrimidin-4-yl)piperazine-1-carboxylate (3)

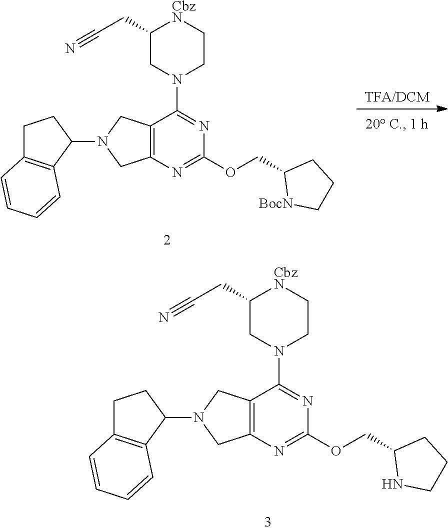 [SMILES: CC(C)(C)OC(=O)N1CCC[C@H]1COc1nc2c(c(N3CCN(C(=O)OCc4ccccc4)[C@@H](CC#N)C3)n1)CN(C1CCc3ccccc31)C2 ; "TFA/DCM" ; N#CC[C@H]1CN(c2nc(OC[C@@H]3CCCN3)nc3c2CN(C2CCc4ccccc42)C3)CCN1C(=O)OCc1ccccc1]

To a mixture of benzyl (2S)-4-(2-(((S)-1-(tert-butoxycarbonyl)pyrrolidin-2-yl)methoxy)-6-(2,3-dihydro-1H-inden-1-yl)-6,7-dihydro-5H-pyrrolo[3,4-d]pyrimidin-4-yl)-2-(cyanomethyl)piperazine-1-carboxylate (2) (1.5 g, 2.16 mmol, 1.0 equiv.) in 20 mL of dichloromethane was added 10 mL of trifluoroacetic acid. The resulting mixture was stirred at 20° C. for 1 h. The mixture was concentrated in vacuo and diluted with saturated NaHCO₃ (20 mL) and extracted with dichloromethane (20 mL*3). The organic layers were combined, dried over anhydrous sodium sulfate, filtered and concentrated in vacuo to give the crude benzyl (2S)-2-(cyanomethyl)-4-(6-(2,3-dihydro-1H-inden-1-yl)-2-(((S)-pyrrolidin-2-yl)methoxy)-6,7-dihydro-5H-pyrrolo[3,4-d]pyrimidin-4-yl)piperazine-1-carboxylate (3) which was used directly in next step without further purification. ESI-MS m/z=594.3 [M+H]⁺. Calculated MW: 593.26.

### Step 3: Synthesis of benzyl (2S)-2-(cyanomethyl)-4-(2-(((S)-1-cyclopropylpyrrolidin-2-yl)methoxy)-6-(2,3-dihydro-1H-inden-1-yl)-6,7-dihydro-5H-pyrrolo[3,4-d]pyrimidin-4-yl)piperazine-1-carboxylate (4)

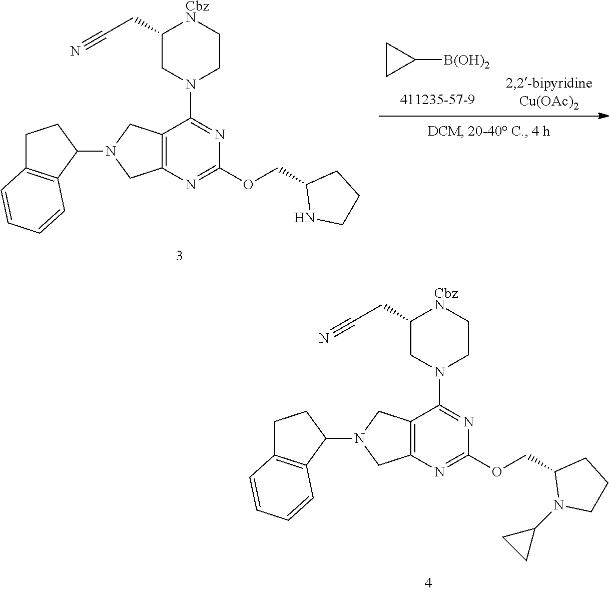

To a mixture of benzyl (2S)-2-(cyanomethyl)-4-(2-(((S)-1-cyclopropylpyrrolidin-2-yl)methoxy)-6-(2,3-dihydro-1H-inden-1-yl)-6,7-dihydro-5H-pyrrolo[3,4-d]pyrimidin-4-yl)piperazine-1-carboxylate (3) (750 mg, 1.26 mmol, 1 equiv.), cyclopropylboronic acid (217 mg, 2.53 mmol, 2 equiv.) and Na₂CO₃ (268 mg, 2.53 mmol, 2 equiv.) in dichloromethane (10 mL) was added 2-(pyridin-2-yl)pyridine (197 mg, 1.26 mmol, 1 equiv.) and Cu(OAc)₂ (69 mg, 0.38 mmol, 0.3 equiv.) at 20° C. Then the resulting mixture was stirred at 40° C. for 4 h under open air. The reaction was slowly quenched with 25% aqueous ammonium hydroxide, extracted with dichloromethane (20 mL*3) and washed with brine. The organic phases were combined, dried over sodium sulfate, filtered, and concentrated in vacuo. The residue was purified by chromatography silica gel (dichloromethane:methanol=10:1) to give benzyl (2S)-2-(cyanomethyl)-4-(2-(((S)-1-cyclopropylpyrrolidin-2-yl)methoxy)-6-(2,3-dihydro-1H-inden-1-yl)-6,7-dihydro-5H-pyrrolo[3,4-d]pyrimidin-4-yl)piperazine-1-carboxylate (4) (293 mg, 36.6% yield) as white solid. ESI-MS m/z=634.3 [M+H]⁺. Calculated MW: 633.34.

### Step 4: Synthesis of 2-((2S)-4-(2-(((S)-1-cyclopropylpyrrolidin-2-yl)methoxy)-6-(2,3-dihydro-1H-inden-1-yl)-6,7-dihydro-5H-pyrrolo[3,4-d]pyrimidin-4-yl)piperazin-2-yl)acetonitrile (5)

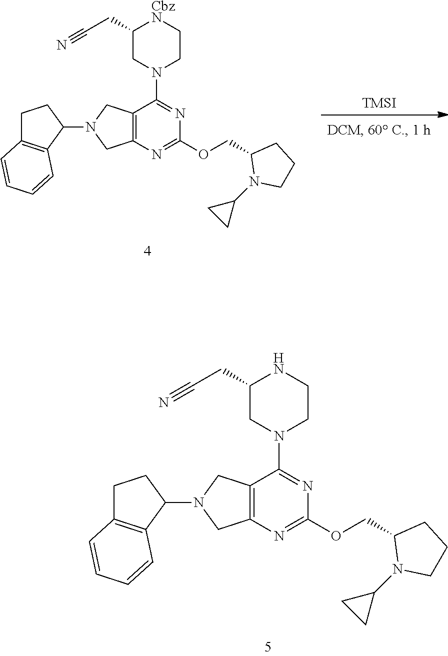 [SMILES: "TMSI" ; N#CC[C@H]1CN(c2nc(OC[C@@H]3CCCN3C3CC3)nc3c2CN(C2CCc4ccccc42)C3)CCN1C(=O)OCc1ccccc1 ; N#CC[C@H]1CN(c2nc(OC[C@@H]3CCCN3C3CC3)nc3c2CN(C2CCc4ccccc42)C3)CCN1]

To a mixture of benzyl (2S)-2-(cyanomethyl)-4-(2-(((S)-1-cyclopropylpyrrolidin-2-yl)methoxy)-6-(2,3-dihydro-1H-inden-1-yl)-6,7-dihydro-5H-pyrrolo[3,4-d]pyrimidin-4-yl)piperazine-1-carboxylate (4) (293 mg, 0.46 mmol, 1 equiv.) in dichloromethane (3 mL) was added trimethylsilyl iodide (462 mg, 2.31 mmol, 5 equiv.) at 20° C. Then the resulting mixture was stirred at 60° C. for 1 h. The reaction mixture was quenched by methanol and the residue was purified by reversed phase column chromatography to give 2-((2S)-4-(2-(((S)-1-cyclopropylpyrrolidin-2-yl)methoxy)-6-(2,3-dihydro-1H-inden-1-yl)-6,7-dihydro-5H-pyrrolo[3,4-d]pyrimidin-4-yl)piperazin-2-yl)acetonitrile (5) (122 mg, 52.8% yield) as yellow solid. ESI-MS m/z=500.1 [M+H]⁺. Calculated MW: 499.31.

### Step 5: Synthesis of 2-((2S)-1-acryloyl-4-(2-(((S)-1-cyclopropylpyrrolidin-2-yl)methoxy)-6-(2,3-dihydro-1H-inden-1-yl)-6,7-dihydro-5H-pyrrolo[3,4-d]pyrimidin-4-yl)piperazin-2-yl)acetonitrile (198)

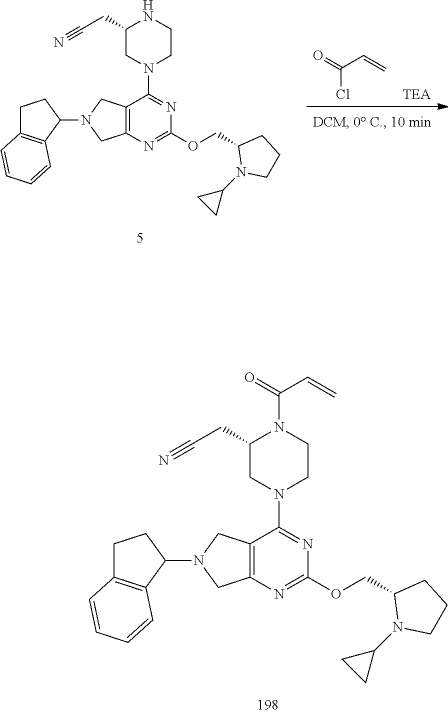

To a mixture of 2-((2S)-4-(2-(((S)-1-cyclopropylpyrrolidin-2-yl)methoxy)-6-(2,3-dihydro-1H-inden-1-yl)-6,7-dihydro-5H-pyrrolo[3,4-d]pyrimidin-4-yl)piperazin-2-yl)acetonitrile (5) (122 mg, 0.24 mmol, 1 equiv.) and triethylamine (124 mg, 1.22 mmol, 5 equiv.) in dichloromethane (3 mL) was added dropwise acryloyl chloride (33 mg, 0.36 mmol, 1.5 equiv.) at 0° C. After addition, the resulting mixture was stirred under nitrogen at 0° C. for 10 minutes. The reaction was quenched with water (10 mL) and extracted with dichloromethane (10 mL×3). The organic phases were combined, dried over sodium sulfate, filtered and evaporated to dryness. The residue was purified by preparative-HPLC to give 2-((2S)-1-acryloyl-4-(2-(((S)-1-cyclopropylpyrrolidin-2-yl)methoxy)-6-(2,3-dihydro-1H-inden-1-yl)-6,7-dihydro-5H-pyrrolo[3,4-d]pyrimidin-4-yl)piperazin-2-yl)acetonitrile (198) (15.47 mg, 11.4% yield, 90.07% purity) as a white powder. ESI-MS m/z=554.3 [M+H]⁺. Calculated MW: 553.32.

### Representative Procedure Q. Synthesis of 1-224.

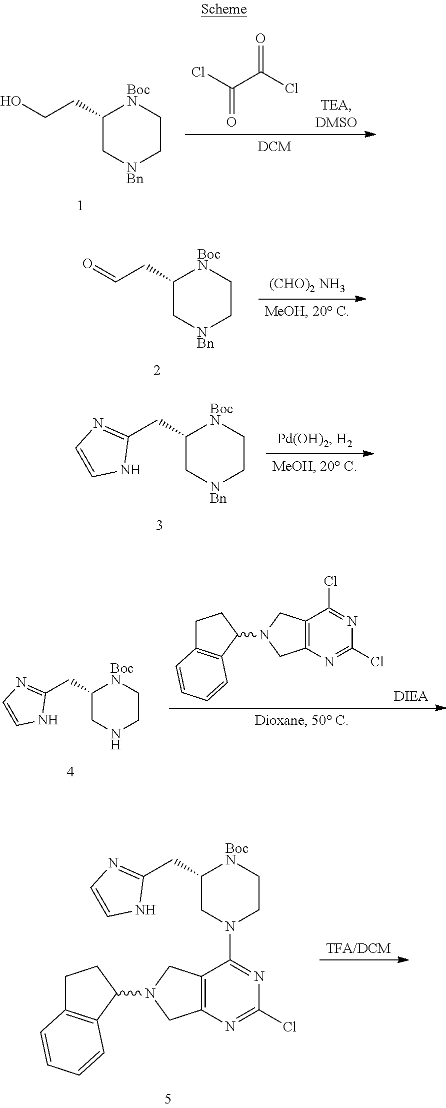

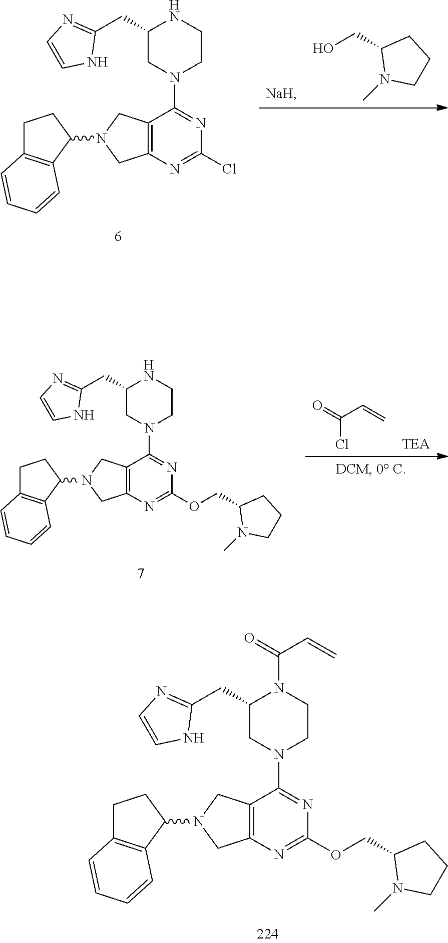

### Step 1: Synthesis of tert-butyl (S)-4-benzyl-2-(2-oxoethyl)piperazine-1-carboxylate (2)

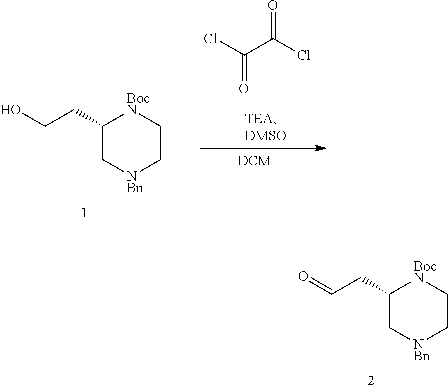 [SMILES: O=C(Cl)C(=O)Cl ; CC(C)(C)OC(=O)N1CCN(Cc2ccccc2)C[C@@H]1CCO ; CC(C)(C)OC(=O)N1CCN(Cc2ccccc2)C[C@@H]1CC=O]

To a solution of oxalyl chloride (1.31 g, 10.3 mmol, 1.1 equiv.) in dichloromethane (20 mL) was added dimethyl sulfoxide (5.79 g, 20.6 mmol, 2.2 equiv.) slowly at −78° C. under nitrogen. After 5 min, a solution of tert-butyl (2S)-4-benzyl-2-(2-hydroxyethyl)piperazine-1-carboxylate (1) (3 g, 9.4 mmol, 1 equiv.) in dichloromethane (5 mL) was added into the reaction mixture slowly at −78° C. and stirred at −78° C. for 0.5 h. Triethylamine (6.5 mL) was then slowly added to the mixture at −78° C. and then the mixture was stirred at −78° C. to 20° C. during a period of 2 h. The reaction mixture was quenched with water (50 mL), extracted with ethyl acetate (15 mL×3) and washed with brine (30 mL). The organic phases were combined, dried over sodium sulfate, filtered and concentrated in vacuo. The residue was purified by silica gel chromatography (15% ethyl acetate) to give tert-butyl (S)-4-benzyl-2-(2-oxoethyl)piperazine-1-carboxylate (2) (1.6 g, 47.8% yield) as colorless gel. ESI-MS m/z=319.3 [M+H]⁺. Calculated MW: 318.19.

### Step 2: Synthesis of tert-butyl (S)-2-((1H-imidazol-2-yl)methyl)-4-benzylpiperazine-1-carboxylate (3)

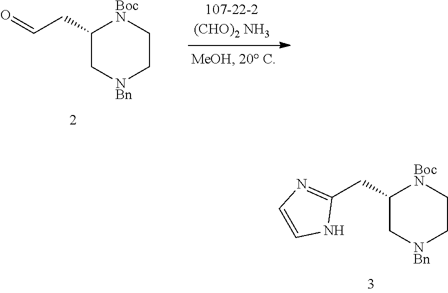 [SMILES: CC(C)(C)OC(=O)N1CCN(Cc2ccccc2)C[C@@H]1CC=O ; CC(C)(C)OC(=O)N1CCN(Cc2ccccc2)C[C@@H]1Cc1ncc[nH]1]

To a solution of tert-butyl (S)-4-benzyl-2-(2-oxoethyl)piperazine-1-carboxylate (2) (1.6 g, 0.4 mol, 1 equiv.) in methanol (5 mL) was added glyoxal (10 mL), ammonia in methanol (10 mL) at 20° C., and the reaction mixture was stirred at 20° C. in a sealed tube for 16 h. The mixture was concentrated in vacuo. Water (50 mL) was added, and the mixture was extracted with dichloromethane (20 ml*3). The combined organic layers were washed with brine (30 mL), dried over sodium sulfate, filtered and concentrated in vacuo. The residue was purified by silica gel chromatography (dichloromethane/methanol=10:1) to give tert-butyl (S)-2-((1H-imidazol-2-yl)methyl)-4-benzylpiperazine-1-carboxylate (3) (1.33 g, 73.0% yield) as brown solid. ESI-MS m/z=357.3 [M+H]⁺. Calculated MW: 356.22.

### Step 3: Synthesis of tert-butyl (S)-2-((1H-imidazol-2-yl)methyl)piperazine-1-carboxylate (4)

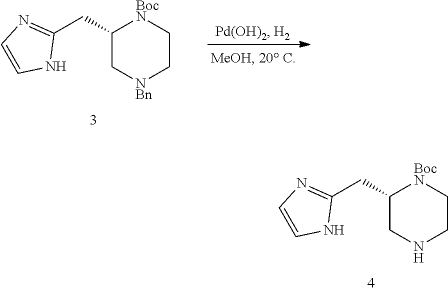 [SMILES: CC(C)(C)OC(=O)N1CCN(Cc2ccccc2)C[C@@H]1Cc1ncc[nH]1 ; CC(C)(C)OC(=O)N1CCNC[C@@H]1Cc1ncc[nH]1]

To a solution of tert-butyl (S)-2-((1H-imidazol-2-yl)methyl)-4-benzylpiperazine-1-carboxylate (3) (1 g, 2.8 mmol, 1 equiv.) in tetrahydrofuran (30 mL) was added Pd(OH)₂/C (1 g) at 20° C. The reaction mixture was stirred at 20° C. for 24 h under an atmosphere of hydrogen. The mixture was filtered and the filtrate was concentrated under reduced pressure to give tert-butyl (S)-2-((1H-imidazol-2-yl)methyl)piperazine-1-carboxylate (4) (718 mg, 67.8% yield) as black solid. ESI-MS m/z=267.1 [M+H]⁺. Calculated MW: 266.17.

### Step 4: Synthesis of tert-butyl (2S)-2-((1H-imidazol-2-yl)methyl)-4-(2-chloro-6-(2,3-dihydro-1H-inden-1-yl)-6,7-dihydro-5H-pyrrolo[3,4-d]pyrimidin-4-yl)piperazine-1-carboxylate (5)

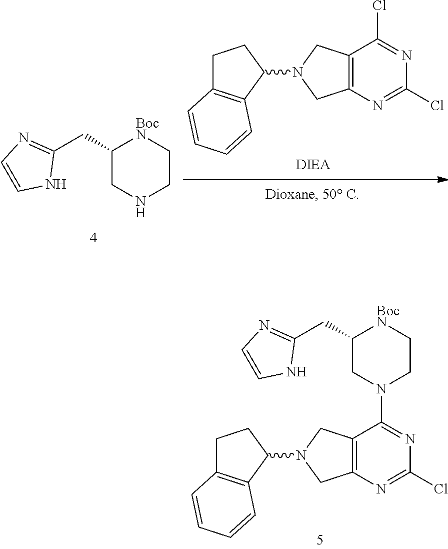

To a solution of tert-butyl (S)-2-((1H-imidazol-2-yl)methyl)piperazine-1-carboxylate (4) (660 mg, 2.47 mmol, 1 equiv.) in dioxane (10 mL) was added 2,4-dichloro-6-(2,3-dihydro-1H-inden-1-yl)-5H,7H-pyrrolo[3,4-d]pyrimidine (758 mg, 2.47 mmol, 1 equiv.) and diisopropylethylamine (0.84 mL). The reaction mixture was stirred at 50° C. for 0.5 h. The reaction mixture was concentrated, diluted with dichloromethane (100 mL) and washed with brine (30 mL×2). The organic phases were combined, dried over sodium sulfate, filtered, and concentrated in vacuo. The residue was purified by silica gel chromatography (dichloromethane/methanol=16:1) to give tert-butyl (2S)-2-((1H-imidazol-2-yl)methyl)-4-(2-chloro-6-(2,3-dihydro-1H-inden-1-yl)-6,7-dihydro-5H-pyrrolo[3,4-d]pyrimidin-4-yl)piperazine-1-carboxylate (5) (1090 mg, 73.8% yield) as white solid. ESI-MS m/z=536.2 [M+H]⁺. Calculated MW: 535.25.

### Step 5: Synthesis of 4-((S)-3-((1H-imidazol-2-yl)methyl)piperazin-1-yl)-2-chloro-6-(2,3-dihydro-1H-inden-1-yl)-6,7-dihydro-5H-pyrrolo[3,4-d]pyrimidine (6)

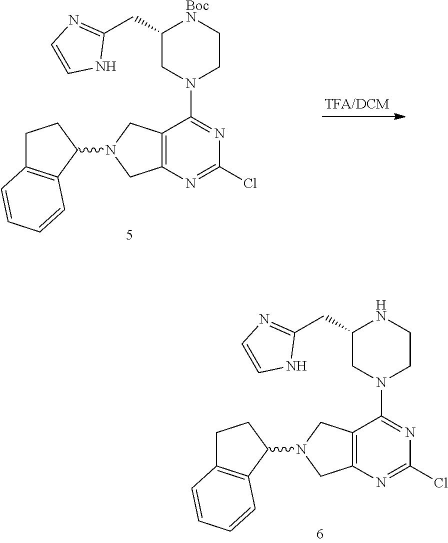 [SMILES: CC(C)(C)OC(=O)N1CCN(c2nc(Cl)nc3c2CN(C2CCc4ccccc42)C3)C[C@@H]1Cc1ncc[nH]1 ; Clc1nc2c(c(N3CCN[C@@H](Cc4ncc[nH]4)C3)n1)CN(C1CCc3ccccc31)C2]

To a mixture of tert-butyl (2S)-2-((1H-imidazol-2-yl)methyl)-4-(2-chloro-6-(2,3-dihydro-1H-inden-1-yl)-6,7-dihydro-5H-pyrrolo[3,4-d]pyrimidin-4-yl)piperazine-1-carboxylate (5) (400 mg, 0.75 mmol, 1 equiv.) in dichloromethane (5 mL) was added trifluoroacetic acid (2.5 mL) at 20° C. The resulting mixture was stirred at 20° C. for 1 h. The reaction mixture was concentrated in vacuo to give 4-((S)-3-((1H-imidazol-2-yl)methyl)piperazin-1-yl)-2-chloro-6-(2,3-dihydro-1H-inden-1-yl)-6,7-dihydro-5H-pyrrolo[3,4-d]pyrimidine (6) (325 mg, 79.9% yield) as brown gel directly used in the next step without further purification. ESI-MS m/z=436.1 [M+H]⁺. Calculated MW: 435.19.

### Step 6: Synthesis of 4-((S)-3-((1H-imidazol-2-yl)methyl)piperazin-1-yl)-6-(2,3-dihydro-1H-inden-1-yl)-2-(((S)-1-methylpyrrolidin-2-yl)methoxy)-6,7-dihydro-5H-pyrrolo[3,4-d]pyrimidine (7)

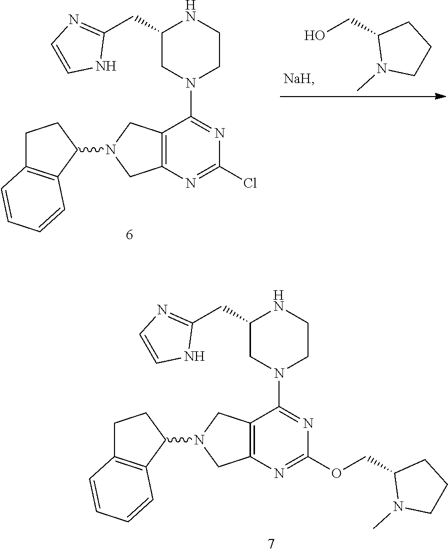

To a mixture of (S)-(1-methylpyrrolidin-2-yl)methanol (858 mg, 7.45 mmol, 10 equiv.) in tetrahydrofuran (6 mL) was added 60% sodium hydride (178 mg, 7.45 mmol, 10 equiv.) at 20° C. After addition, the resulting mixture was stirred at 20° C. for 15 minutes, and then 4-((S)-3-((1H-imidazol-2-yl)methyl)piperazin-1-yl)-2-chloro-6-(2,3-dihydro-1H-inden-1-yl)-6,7-dihydro-5H-pyrrolo[3,4-d]pyrimidine (6) (325 mg, 0.74 mmol, 1 equiv.) was added into the reaction mixture and stirring continued at 80° C. for 16 h. The reaction mixture was quenched with saturated NH₄Cl (15 mL) and concentrated in vacuo. The residue was extracted with dichloromethane (10 mL×3). The organic phases were combined, dried over sodium sulfate, filtered and evaporated to dryness. The residue was purified by preparative-thin layer chromatography (dichloromethane/methanol=5:1) to give 4-((S)-3-((1H-imidazol-2-yl)methyl)piperazin-1-yl)-6-(2,3-dihydro-1H-inden-1-yl)-2-(((S)-1-methylpyrrolidin-2-yl)methoxy)-6,7-dihydro-5H-pyrrolo[3,4-d]pyrimidine (7) (259 mg, 67.6% yield) as brown oil. ESI-MS m/z=515.3 [M+H]⁺. Calculated MW: 514.32.

### Step 7: Synthesis of 1-((2S)-2-((1H-imidazol-2-yl)methyl)-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)piperazin-1-yl)prop-2-en-1-one (224)

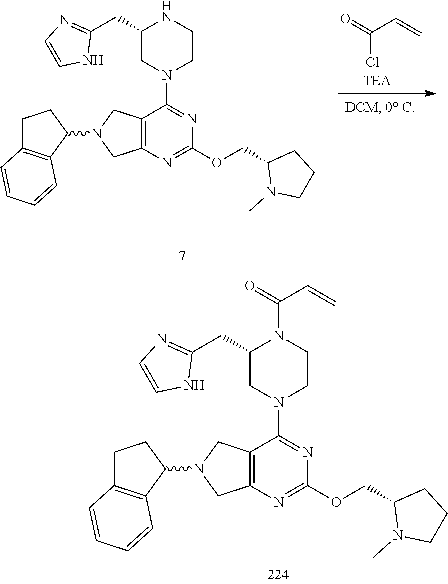

To a mixture of 4-((S)-3-((1H-imidazol-2-yl)methyl)piperazin-1-yl)-6-(2,3-dihydro-1H-inden-1-yl)-2-(((S)-1-methylpyrrolidin-2-yl)methoxy)-6,7-dihydro-5H-pyrrolo[3,4-d]pyrimidine (7) (259 mg, 0.5 mmol, 1 equiv.) and triethylamine (254 mg, 2.5 mmol, 5 equiv.) in dichloromethane (5 mL) was added dropwise acryloyl chloride (45 mg, 0.5 mmol, 1 equiv.) at 0° C. The resulting mixture was stirred at 0° C. for 10 min. The reaction was quenched with water (20 mL), extracted with dichloromethane (20 mL×3). The organic phases were combined, dried over sodium sulfate, and filtered. The filtrate was evaporated to dryness and the residue was purified by reversed flash column (10% acetonitrile) to give 1-((2S)-2-((1H-imidazol-2-yl)methyl)-4-(6-(2,3-dihydro-1H-inden-1-yl)-2-(((S)-1-methylpyrrolidin-2-yl)methoxy)-6,7-dihydro-5H-pyrrolo[3,4-d]pyrimidin-4-yl)piperazin-1-yl)prop-2-en-1-one (224) (20 mg, 6.92% yield) as colorless gel. ESI-MS m/z=569.3 [M+H]⁺. Calculated MW: 568.33.

### Representative Procedure R. Synthesis of 1-304.

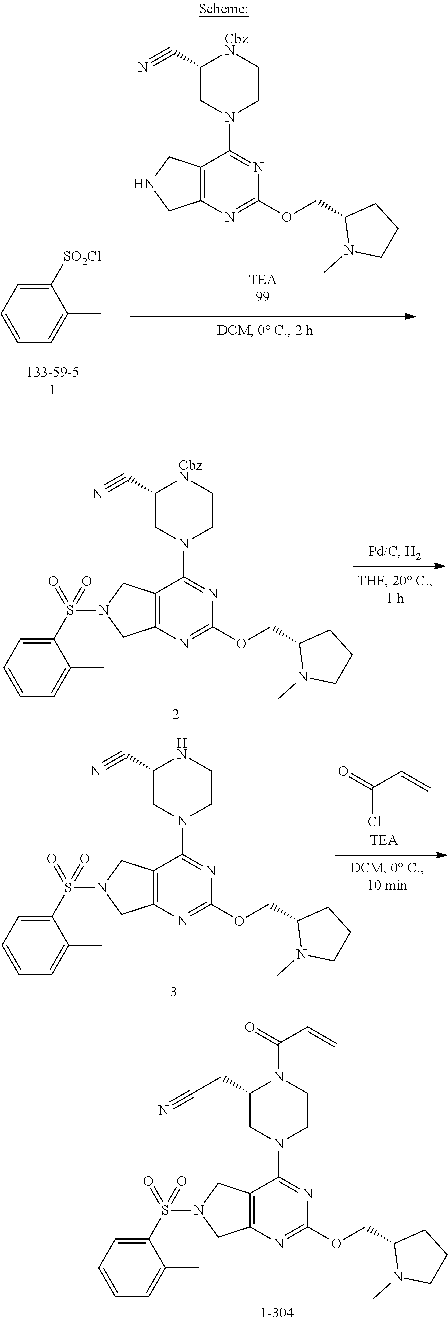

### Step 1: Synthesis of benzyl (2S)-2-(cyanomethyl)-4-{6-[(2-methylbenzene)sulfonyl]-2-{[(2S)-1-methylpyrrolidin-2-yl]methoxy}-5H,7H-pyrrolo[3,4-d]pyrimidin-4-yl}piperazine-1-carboxylate (2)

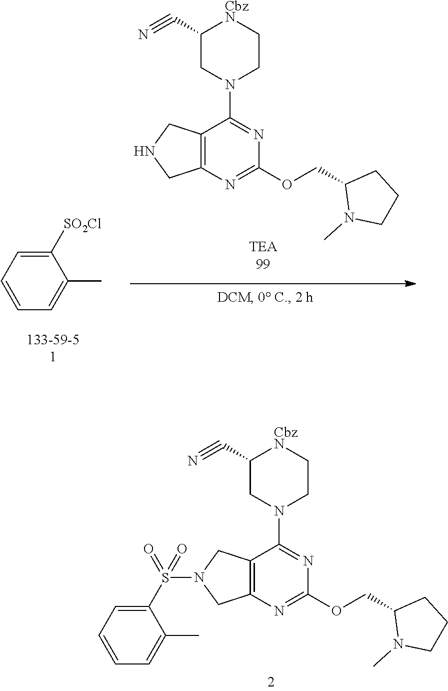 [SMILES: Cc1ccccc1S(=O)(=O)Cl ; CN1CCC[C@H]1COc1nc2c(c(N3CCN(C(=O)OCc4ccccc4)[C@@H](C#N)C3)n1)CNC2 ; Cc1ccccc1S(=O)(=O)N1Cc2nc(OC[C@@H]3CCCN3C)nc(N3CCN(C(=O)OCc4ccccc4)[C@@H](C#N)C3)c2C1]

To a solution of benzyl (2S)-2-(cyanomethyl)-4-(2-{[(2S)-1-methylpyrrolidin-2-yl]methoxy}-5H,6H,7H-pyrrolo[3,4-d]pyrimidin-4-yl)piperazine-1-carboxylate (99) (320 mg, 0.65 mmol, 1 equiv., for the procedure of 99, please refer to the procedure of compound 1-11) and triethylamine (132 mg, 1.30 mmol, 2 equiv.) in dichloromethane (10 mL) was added dropwise of 2-methylbenzenesulfonyl chloride (137 mg, 0.72 mmol, 1.1 equiv.) at 0° C. After addition, the resulting mixture was stirred at 0° C. for 2 h. Then to reaction mixture was added water (10 mL) and extracted with dichloromethane (30 mL×3). The organic phases were combined and dried over sodium sulfate. The combined organic layer was washed with brine (20 mL), dried over sodium sulfate, filtered and concentrated in vacuo to give a residue. The residue was purified by prep-thin layer chromatography (dichloromethane/methanol=10:1) followed by lyophilization to yield benzyl (2S)-2-(cyanomethyl)-4-{6-[(2-methylbenzene)sulfonyl]-2-{[(2S)-1-methylpyrrolidin-2-yl]methoxy}-5H,7H-pyrrolo[3,4-d]pyrimidin-4-yl}piperazine-1-carboxylate (2) (311 mg, 67.4% yield) as white solid. ESI-MS m/z=646.30 [M+H]⁺. Calculated MW: 645.27.

### Step 2: Synthesis of 2-[(2S)-4-{6-[(2-methylbenzene)sulfonyl]-2-{[(2S)-1-methylpyrrolidin-2-yl]methoxy}-5H,7H-pyrrolo[3,4-d]pyrimidin-4-yl}piperazin-2-yl]acetonitrile (3)

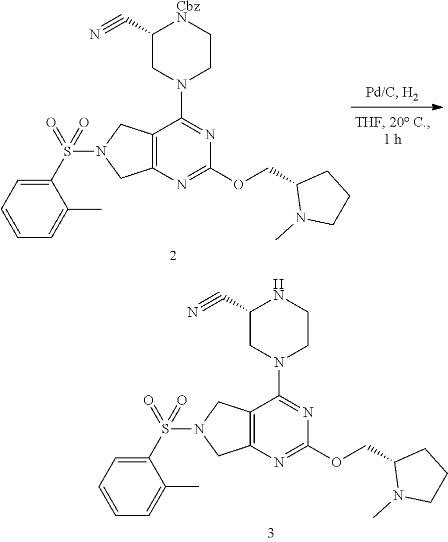 [SMILES: Cc1ccccc1S(=O)(=O)N1Cc2nc(OC[C@@H]3CCCN3C)nc(N3CCN(C(=O)OCc4ccccc4)[C@@H](C#N)C3)c2C1 ; Cc1ccccc1S(=O)(=O)N1Cc2nc(OC[C@@H]3CCCN3C)nc(N3CCN[C@@H](C#N)C3)c2C1]

To a solution of benzyl (2S)-2-(cyanomethyl)-4-{6-[(2-methylbenzene)sulfonyl]-2-{[(2S)-1-methylpyrrolidin-2-yl]methoxy}-5H,7H-pyrrolo[3,4-d]pyrimidin-4-yl}piperazine-1-carboxylate (311 mg, 0.48 mmol, 1 equiv) in tetrahydrofuran (10 mL) was added 10% Pd/C (300 mg). The mixture was then stirred at 20° C. under hydrogen (1 atm) for 1 h. The reaction was filtered and the filtrate was concentrated in vacuo. The residue was purified by per-thin layer chromatography (dichloromethane/methanol=10:1) to give 2-[(2S)-4-{6-[(2-methylbenzene)sulfonyl]-2-{[(2S)-1-methylpyrrolidin-2-yl]methoxy}-5H,7H-pyrrolo[3,4-d]pyrimidin-4-yl}-1-(prop-2-enoyl)piperazin-2-yl]acetonitrile (3) (232 mg, 94.1% yield) as yellow solid. ESI-MS m/z=512.30 [M+H]⁺. Calculated MW: 511.24.

### Step 3: Synthesis of 2-[(2S)-4-{6-[(2-methylbenzene)sulfonyl]-2-{[(2S)-1-methylpyrrolidin-2-yl]methoxy}-5H,7H-pyrrolo[3,4-d]pyrimidin-4-yl}-1-(prop-2-enoyl)piperazin-2-yl]acetonitrile (1-304)

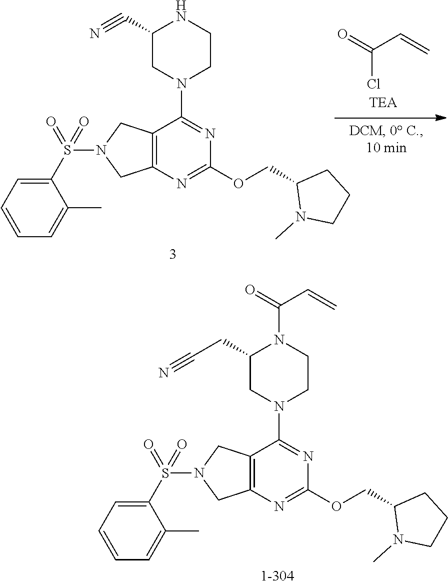

To a solution of 2-[(2S)-4-{6-[(2-methylbenzene)sulfonyl]-2-{[(2S)-1-methylpyrrolidin-2-yl]methoxy}-5H,7H-pyrrolo[3,4-d]pyrimidin-4-yl}piperazin-2-yl]acetonitrile (3) (232 mg, 0.45 mmol, 1 equiv.) and triethylamine (137 mg, 1.36 mmol, 3 equiv.) in dichloromethane (5 mL) was added dropwise of prop-2-enoyl chloride (49 mg, 0.54 mmol, 1.2 equiv.) at 0° C. After addition, the resulting mixture was stirred at 0° C. for 10 min. Then to reaction mixture was added water (10 mL), extracted with dichloromethane (30 mL×3). The organic phase was combined, and dried over sodium sulfate, filtered. The filtrate was evaporated to dryness and the residue was purified by neutral reverse phase high pressure liquid chromatography (water/acetonitrile=20:1) to give 2-[(2S)-4-{6-[(2-methylbenzene)sulfonyl]-2-{[(2S)-1-methylpyrrolidin-2-yl]methoxy}-5H,7H-pyrrolo[3,4-d]pyrimidin-4-yl}-1-(prop-2-enoyl)piperazin-2-yl]acetonitrile (32 mg, 12.3% yield) as white solid. ESI-MS m/z=566.30 [M+H]⁺. Calculated MW: 565.25.

### Representative Procedure S. Synthesis of 1-326.

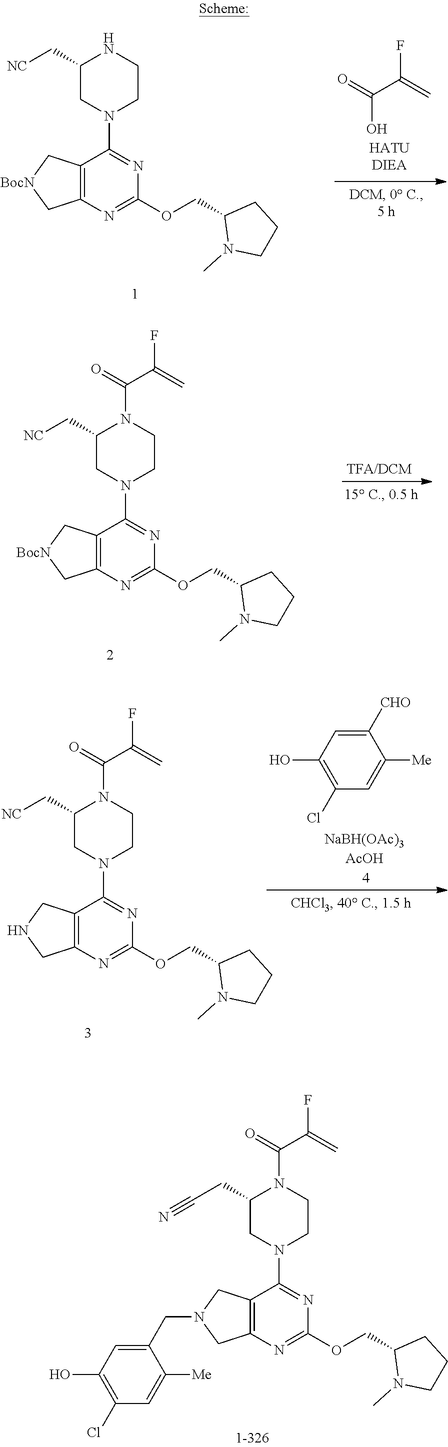

### Step 1: Synthesis of tert-butyl 4-[(3S)-3-(cyanomethyl)-4-(2-fluoroprop-2-enoyl)piperazin-1-yl]-2-{[(2S)-1-methylpyrrolidin-2-yl]methoxy}-5H,7H-pyrrolo[3,4-d]pyrimidine-6-carboxylate (2)

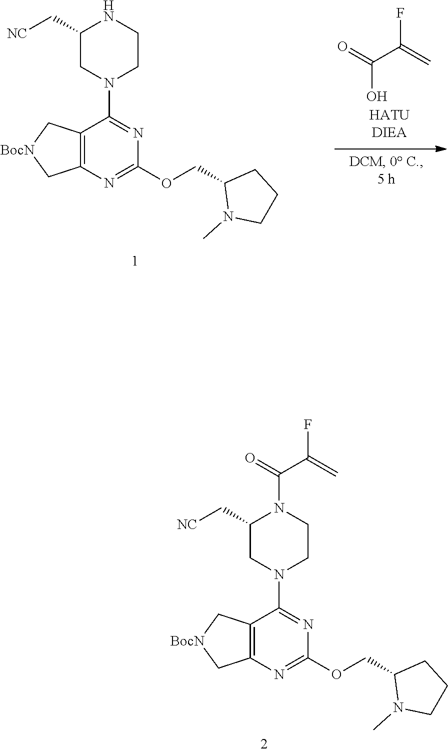

To a mixture of 2-fluoroprop-2-enoic acid (79 mg, 0.87 mmol, 1 equiv.) in dimethylformamide (8 mL) was added DIEA (339 mg, 2.62 mmol, 3 equiv.), tert-butyl 4-[(3S)-3-(cyanomethyl)piperazin-1-yl]-2-{[(2S)-1-methylpyrrolidin-2-yl]methoxy}-5H,7H-pyrrolo[3,4-d]pyrimidine-6-carboxylate (1) (400 mg, 0.87 mmol, 1 equiv.) and HATU (499 mg, 1.31 mmol, 1.5 equiv.) at 0° C. The mixture was then stirred at 0° C. for 0.5 h. The reaction was quenched with water (80 mL) and extracted with ethyl acetate (50 mL×3). The organic phase was combined, dried over sodium sulfate, filtered, and concentrated in vacuo. The residue was purified by preparative-thin layer chromatography (dichloromethane/methanol=10:1) to give tert-butyl 4-[(3S)-3-(cyanomethyl)-4-(2-fluoroprop-2-enoyl)piperazin-1-yl]-2-{[(2S)-1-methylpyrrolidin-2-yl]methoxy}-5H,7H-pyrrolo[3,4-d]pyrimidine-6-carboxylate (2) (347 mg, 75% yield) as brown oil. ESI-MS m/z=530.3[M+H]⁺. Calculated MW: 529.28.

### Step 2: Synthesis of 2-[(2S)-1-(2-fluoroprop-2-enoyl)-4-(2-{[(2S)-1-methylpyrrolidin-2-yl]methoxy}-5H,6H,7H-pyrrolo[3,4-d]pyrimidin-4-yl)piperazin-2-yl]acetonitrile (3)

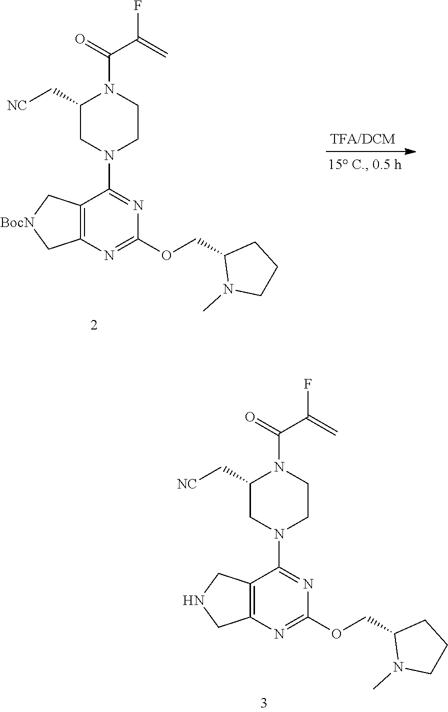 [SMILES: C=C(F)C(=O)N1CCN(c2nc(OC[C@@H]3CCCN3C)nc3c2CN(C(=O)OC(C)(C)C)C3)C[C@@H]1CC#N ; C=C(F)C(=O)N1CCN(c2nc(OC[C@@H]3CCCN3C)nc3c2CNC3)C[C@@H]1CC#N]

To a mixture of tert-butyl 4-[(3S)-3-(cyanomethyl)-4-(2-fluoroprop-2-enoyl)piperazin-1-yl]-2-{[(2S)-1-methylpyrrolidin-2-yl]methoxy}-5H,7H-pyrrolo[3,4-d]pyrimidine-6-carboxylate (2) (347 mg, 0.66 mmol, 1 equiv.) in dichloromethane (5 mL) was added trifluoroacetic acid (2.5 mL) at 15° C., then the mixture was stirred at 15° C. for 0.5 h. The reaction was concentrated in vacuo to give 2-[(2S)-1-(2-fluoroprop-2-enoyl)-4-(2-{[(2S)-1-methylpyrrolidin-2-yl]methoxy}-5H,6H,7H-pyrrolo[3,4-d]pyrimidin-4-yl)piperazin-2-yl]acetonitrile (3) (184 mg, 65% yield) as yellow solid. ESI-MS m/z=430.3[M+H]⁺. Calculated MW: 429.23.

### Step 3: 2-[(2S)-4-{6-[(4-chloro-5-hydroxy-2-methylphenyl)methyl]-2-{[(2S)-1-methylpyrrolidin-2-yl]methoxy}-5H,7H-pyrrolo[3,4-d]pyrimidin-4-yl}-1-(2-fluoroprop-2-enoyl)piperazin-2-yl]acetonitrile (1-326)

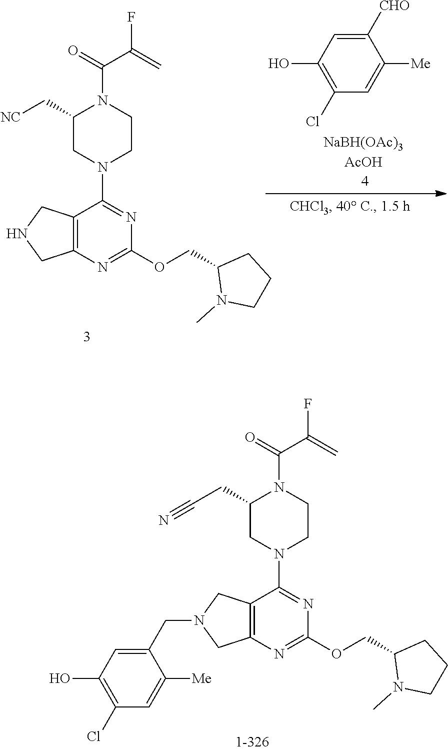 [SMILES: C=C(F)C(=O)N1CCN(c2nc(OC[C@@H]3CCCN3C)nc3c2CNC3)C[C@@H]1CC#N ; Cc1cc(Cl)c(O)cc1C=O ; C=C(F)C(=O)N1CCN(c2nc(OC[C@@H]3CCCN3C)nc3c2CN(Cc2cc(O)c(Cl)cc2C)C3)C[C@@H]1CC#N]

To a mixture of 2-[(2S)-1-(2-fluoroprop-2-enoyl)-4-(2-{[(2S)-1-methylpyrrolidin-2-yl]methoxy}-5H,6H,7H-pyrrolo[3,4-d]pyrimidin-4-yl)piperazin-2-yl]acetonitrile (3) (184 mg, 0.43 mmol, 1 equiv.) in chloroform (5 mL) was added 4-chloro-5-hydroxy-2-methylbenzaldehyde (4) (110 mg, 0.64 mmol, 1.5 equiv.) and acetic acid (0.5 mL), then sodium triacetoxyborohydride (273 mg, 1.29 mmol, 3 equiv.) was added slowly to the above mixture at 15° C. The mixture was then stirred at 40° C. for 1.5 h. The reaction was quenched with water (30 mL) and extracted with dichloromethane (30 mL*3). The organic layers were combined, dried over anhydrous sodium sulfate, filtered and the filtrate was concentrated in vacuo. The residue was purified by preparative-high pressure liquid chromatography to give 2-[(2S)-4-{6-[(4-chloro-5-hydroxy-2-methylphenyl)methyl]-2-{[(2S)-1-methylpyrrolidin-2-yl]methoxy}-5H,7H-pyrrolo[3,4-d]pyrimidin-4-yl}-1-(2-fluoroprop-2-enoyl)piperazin-2-yl]acetonitrile (1-326) (99.1 mg, 40% yield) as white solid. ESI-MS m/z=584.2[M+H]⁺. Calculated MW: 583.25.

### Example 2

This Example provides a protocol for assessing covalent adduct formation (CAF) between the compounds shown in Table 1 above and KRAS.

In vitro covalent adduct formation assay: Covalent adduct formation (CAF) reactions between Cys12 of the KRAS 4B G12C protein and some of the compounds of Table 1 were measured in vitro using liquid chromatography-mass spectrometry (LC-MS).

Recombinant Human KRAS 4B protein containing the G12C mutation was used in compound screening experiments. This protein contained 188 amino acids in total, including an N-terminal 6-Histidine tag, followed by a Tobacco Etch Virus (TEV) tag, followed by residues 1-169 of the native KRAS 4B sequence. The exact mass of the protein was 21,310 Da as determined by mass spectrometry. The full amino acid sequence is shown below:

(SEQ ID NO.: 4)
MAHHHHHHAG GAENLYFQSM TEYKLVVVGA CGVGKSALTI
QLIQNHFVDE YDPTIEDSYR KQVVIDGETC LLDILDTAGQ
EEYSAMRDQY MRTGEGFLCV FAINNTKSFE DIHHYREQIK
RVKDSEDVPM VLVGNKCDLP SRTVDTKQAQ DLARSYGIPF
IETSAKTRQG VDDAFYTLVR EIRKHKEK

In an alternative screen, the assay can be conducted using a KRAS 4b G12C protein having 170 amino acids, a mass of 19,336 Da, and the amino acid sequence SM

(SERQ ID NO.: 5)
TEYKLVVVGA CGVGKSALTI QLIQNHFVDE YDPTIEDSYR
KQVVIDGETC LLDILDTAGQ EEYSAMRDQY MRTGEGFLCV
FAINNTKSFE DIHHYREQIK RVKDSEDVPM VLVGNKCDLP
SRTVDTKQAQ DLARSYGIPF IETSAKTRQG VDDAFYTLVR
EIRKHKEK.

The recombinant protein was expressed in E. coli BL21 cells and purified using affinity chromatography via a Ni-NTA column. Protein stocks were nucleotide-exchanged to >95% GDP, concentrated to 4 mg/mL, and stored at −80° C. in storage buffer (50 mM HEPES pH 7.4, 50 mM NaCl, 5 mM MgCl2, 1 mM DTT). Pure KRAS 4B G12C protein was diluted to a concentration of 5 μM in Tris Buffered Saline, pH 7.4. The compounds were dissolved in DMSO and added to the diluted protein to make a 10 μM concentration. The total DMSO concentration in the reaction was 4%. The reaction was mixed by pipetting and incubated at 22° C. for one hour. Aliquots of the reaction were taken over time and diluted 2:1 in 0.1% formic acid. The intact mass of the protein samples was measured by LC-MS using a QExactive+ mass spectrometer (Thermo Scientific). An amount of 500 ng total protein was injected onto a C8 reverse phase column, eluted with a seven-minute gradient of 30%-90% acetonitrile/0.1% formic acid, and analyzed for intact mass by the mass spectrometer. Adducts identified were confirmed to be within 1 Dalton of the expected mass, and the relative ratios of free:adduct protein were used to quantify the percentage of protein bound by the compound. CAF reactions were run in duplicate, with a typical variability of ±5%.

What is claimed is:

1.-103. (canceled)

104. A compound of Formula (VI) or pharmaceutically acceptable salt thereof: 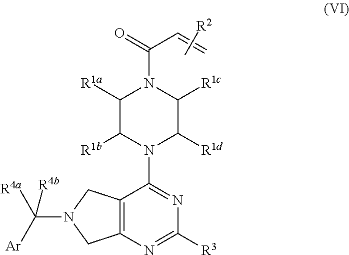
wherein:
Ar is 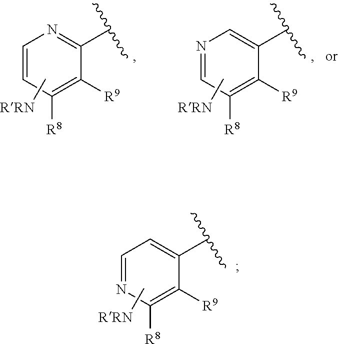
wherein R and R′ are independently hydrogen or C₁-C₄ alkyl; and
R⁸, and R⁹ are each independently selected from the group consisting of hydrogen, halo, alkyl, alkoxy, haloalkyl, trifluoromethyl, cycloalkyl; or R⁸, and R⁹ together combine to form a further fused ring that is an aromatic ring optionally comprising 1 to 3 heteroatoms independently selected from N, O or S, the further fused ring being optionally substituted;
R^{1a}, R^{1b}, R^{1c}, and R^{1d} are independently selected from hydrogen, cyano, alkyl, amido, alkylamido, CH₂SOₚMe, where p is 0 to 2, heteroarylalkyl, hydroxy alkyl, alkynylalkyl and cyanoalkyl; wherein any C—H present in R^{1a}, R^{1b}, R^{1c}, and R^{1d} is optionally exchanged for C—F; or any two R^{1a}, R^{1b}, R^{1c}, and R^{1d} combine to form to form a fused 3-6-membered ring or a 1 to 4 atom bridging unit, wherein each atom of the fused 3-6-membered ring or the 1 to 4 atom bridging unit comprises, independently, an optionally substituted methylene unit or a heteroatom selected from NR^{N}, O, or S, or SO₂, wherein R^{N} is hydrogen, alkyl, and fluorinated alkyl; and wherein the fused 3-6-membered ring or the 1 to 4 atom bridging unit is optionally substituted with oxo, halogen, and hydroxyl;
R² is selected from the group consisting of hydrogen, fluorine, methyl, and —CH₂NR^{a}R^{b}, wherein R^{a} and R^{b} are independently selected from hydrogen or alkyl; or R^{a} and R^{b} combine to form a C₂-C₆ nitrogen containing heterocycle;
R³ is selected from hydrogen, alkyl, alkoxy, amino, aminoalkylamino, halogen, heterocyclylalkoxy, aminoalkoxy, N-alkylaminoalkoxy, N,N-dialkylaminoalkoxy, mercapto-alkyl, mercapto aryl, aryl, any of which may be optionally substituted; and
R^{4a} and R^{4b} are independently selected from hydrogen, aryl, alkyl, trifluoroalkyl, alkyl optionally with halogen and cycloalkyl; or one of R^{4a} and R^{4b} forms a fused, non-aromatic ring structure with Ar.
or R^{4a} and R^{4b} together define a double-bonded oxygen (carbonyl).

105. The compound of any one of claim 104, wherein R³ is O-linked N-methyl-L-prolinol.

106. The compound of any one of claim 104, wherein R³ is hydrogen.

107. The compound of claim 104, wherein R² is hydrogen.

108. The compound of claim 104, wherein R² is fluorine.

109. The compound of claim 104, wherein R² is N,N-dimethylaminomethyl.

110. The compound of claim 104, wherein R^{1b} is methyl.

111. The compound of claim 110, wherein a stereogenic center created by the R^{1b} methyl group is in the R-configuration.

112. The compound of claim 110, wherein a stereogenic center created by the R^{1b} methyl group is in the S-configuration.

113. The compound of claim 104, wherein R^{1c} is methyl.

114. The compound of claim 113, wherein a stereogenic center created by the R^{1c} methyl group is in the R-configuration.

115. The compound of claim 113, wherein a stereogenic center created by the R^{1c} methyl group is in the S-configuration.

116. The compound of claim 104, wherein R^{1a} is cyanomethyl.

117. The compound of claim 116, wherein a stereogenic center created by the cyanomethyl group is in the R-configuration.

118. The compound of claim 116, wherein a stereogenic center created by the cyanomethyl group is in the S-configuration.

119. The compound of claim 104, wherein R^{1d} is hydrogen.

120.-121. (canceled)

122. The compound of claim 104, wherein Ar is selected from the group consisting of:

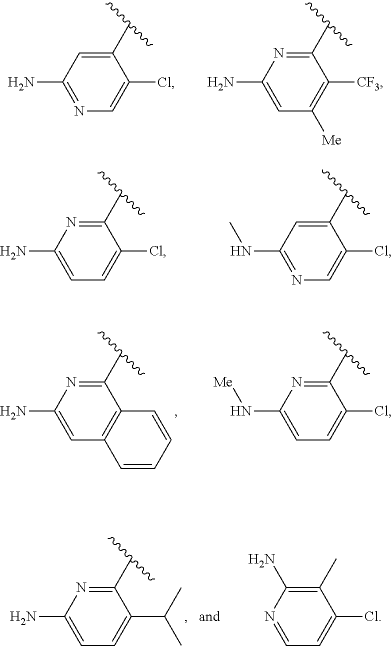

123. A method of modulating a G12C mutant K-Ras comprising contacting the G12C mutant K-Ras with a compound of claim 104.

124. A method of treating a subject with cancer associated with a G12C Kras mutation comprising administering to the subject a compound of claim 104 in a pharmaceutically acceptable vehicle.

125. (canceled)

126. A compound of claim 104, wherein the compound of Formula (VI) is: 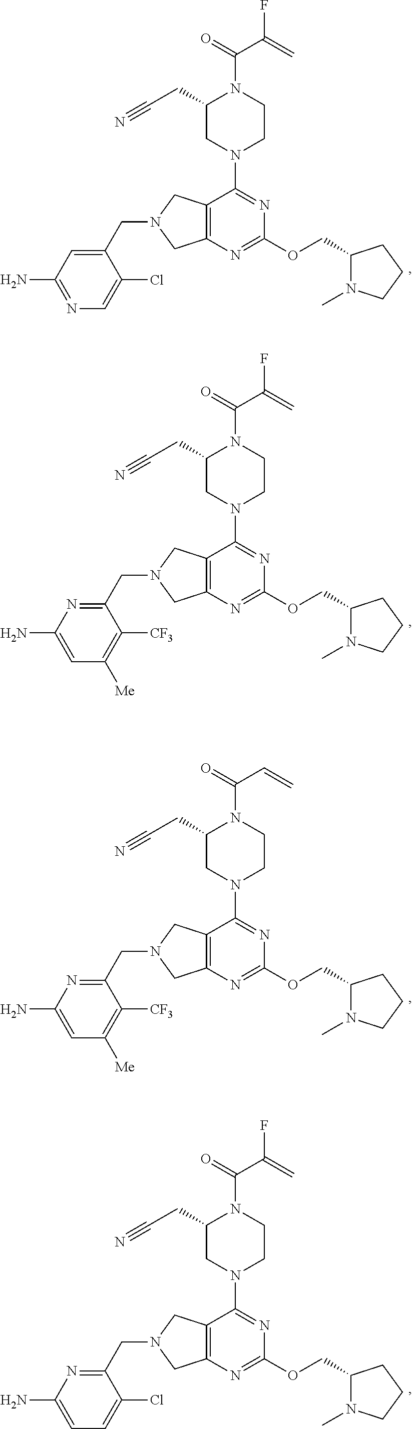 [SMILES: C=C(F)C(=O)N1CCN(c2nc(OC[C@@H]3CCCN3C)nc3c2CN(Cc2cc(N)ncc2Cl)C3)C[C@@H]1CC#N ; C=C(F)C(=O)N1CCN(c2nc(OC[C@@H]3CCCN3C)nc3c2CN(Cc2nc(N)cc(C)c2C(F)(F)F)C3)C[C@@H]1CC#N ; C=CC(=O)N1CCN(c2nc(OC[C@@H]3CCCN3C)nc3c2CN(Cc2nc(N)cc(C)c2C(F)(F)F)C3)C[C@@H]1CC#N ; C=C(F)C(=O)N1CCN(c2nc(OC[C@@H]3CCCN3C)nc3c2CN(Cc2nc(N)ccc2Cl)C3)C[C@@H]1CC#N] 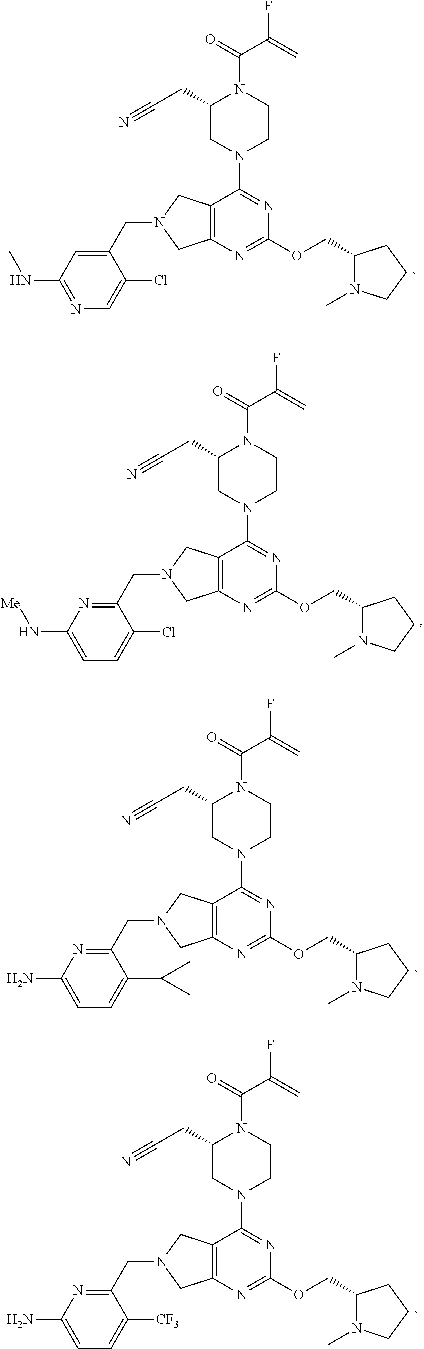 [SMILES: C=C(F)C(=O)N1CCN(c2nc(OC[C@@H]3CCCN3C)nc3c2CN(Cc2cc(NC)ncc2Cl)C3)C[C@@H]1CC#N ; C=C(F)C(=O)N1CCN(c2nc(OC[C@@H]3CCCN3C)nc3c2CN(Cc2nc(NC)ccc2Cl)C3)C[C@@H]1CC#N ; C=C(F)C(=O)N1CCN(c2nc(OC[C@@H]3CCCN3C)nc3c2CN(Cc2nc(N)ccc2C(C)C)C3)C[C@@H]1CC#N ; C=C(F)C(=O)N1CCN(c2nc(OC[C@@H]3CCCN3C)nc3c2CN(Cc2nc(N)ccc2C(F)(F)F)C3)C[C@@H]1CC#N] 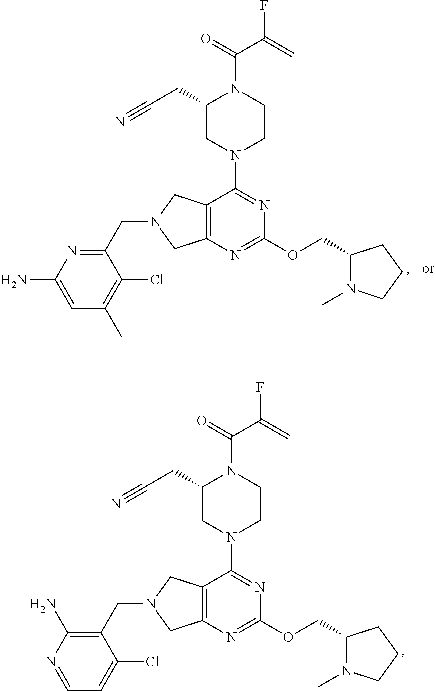 [SMILES: C=C(F)C(=O)N1CCN(c2nc(OC[C@@H]3CCCN3C)nc3c2CN(Cc2nc(N)cc(C)c2Cl)C3)C[C@@H]1CC#N ; C=C(F)C(=O)N1CCN(c2nc(OC[C@@H]3CCCN3C)nc3c2CN(Cc2c(Cl)ccnc2N)C3)C[C@@H]1CC#N]
or a pharmaceutically acceptable salt thereof.
